(19) United States
(12) Patent Application Publication
(10) Pub. No.: US 2025/0059283 A1
(43) Pub. Date: Feb. 20, 2025

(54) CHIMERIC ANTIGEN RECEPTORS TARGETING FC RECEPTOR-LIKE 5 AND USES THEREOF

(71) Applicants: MEMORIAL SLOAN-KETTERING CANCER CENTER, New York, NY (US); EUREKA THERAPEUTICS, INC., Emeryville, CA (US)

(72) Inventors: Renier J. Brentjens, Short Hills, NJ (US); Eric L. Smith, Brookline, MA (US); Cheng Liu, Emeryville, CA (US)

(73) Assignees: MEMORIAL SLOAN-KETTERING CANCER CENTER, New York, NY (US); EUREKA THERAPEUTICS, INC., Emeryville, CA (US)

(21) Appl. No.: 18/909,536

(22) Filed: Oct. 8, 2024

Related U.S. Application Data
(62) Division of application No. 17/332,853, filed on May 27, 2021, now Pat. No. 12,139,538.
Application No. 17/332,853 is a division of application No. 15/614,108, filed on Jun. 5, 2017, now Pat. No. 11,059,891.
Application No. 15/614,108 is a continuation of application No. PCT/US2015/064134, filed on Dec. 4, 2015.
(60) Provisional application No. 62/088,164, filed on Dec. 5, 2014.

(51) Int. Cl. C07K 16/28; A61K 39/00; C07K 14/705; C07K 14/725; C07K 16/30; C12N 5/0783; C12N 9/12; C12N 15/86

(52) U.S. Cl. CPC C07K 16/283; A61K 39/4611; A61K 39/4631; A61K 39/464402; A61K 39/464412; A61K 2239/48; C07K 14/7051; C07K 14/70517; C07K 14/70521; C07K 14/7056; C07K 14/70578; C07K 16/3061; C12N 5/0636; C12N 9/1205; C12N 15/86; A61K 2039/505; C07K 2317/31; C07K 2317/34; C07K 2317/54; C07K 2317/55; C07K 2317/56; C07K 2317/565; C07K 2317/622; C07K 2317/92; C07K 2319/02; C07K 2319/03; C07K 2319/33; C07K 2319/40; C07K 2319/74; C12N 2510/00; C12N 2740/10043; C12N 2740/13043; C12N 2840/203

(57) ABSTRACT

The presently disclosed subject matter provides for methods and compositions for treating a neoplasia (e.g., multiple myeloma). It relates to chimeric antigen receptors (CARs) that specifically target Fc Receptor-like 5 (FcRL5), e.g., domain 9 of FcRL5, and immunoresponsive cells comprising such CARs. The presently disclosed FcRL5-targeted CARs have enhanced immune-activating properties, including anti-tumor activity.

### CROSS-REFERENCE TO RELATED APPLICATIONS

This application is a Divisional of U.S. patent application Ser. No. 17/332,853, filed May 27, 2021, which is a Divisional of U.S. patent application Ser. No. 15/614,108, filed Jun. 5, 2017, which is a Continuation of International Application Serial No. PCT/US2015/064134, filed Dec. 4, 2015, which claims priority to U.S. Provisional Patent Application Ser. No. 62/088,164, filed Dec. 5, 2014, the contents of each of which are incorporated by reference in their entirety, and to each of which priority is claimed.

### SEQUENCE LISTING

The specification further incorporates by reference the Sequence Listing submitted herewith on Oct. 3, 2024. The Sequence Listing file, identified as 072734.1655.xml, is 1,810,260 bytes and was created on Oct. 3, 2024. The Sequence Listing, electronically filed herewith, does not extend beyond the scope of the specification and thus does not contain new matter.

### INTRODUCTION

The presently disclosed subject matter provides for methods and compositions for treating cancer. It relates to chimeric antigen receptors (CARs) that specifically target Fc Receptor-like 5 (FcRL5), e.g., domain 9 of FcRL5, immunoresponsive cells comprising such CARs, and methods of using such cells for treating cancer (e.g., multiple myeloma).

### BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies.

Multiple myeloma (MM) is the second most common hematologic malignancy mortality (Siegel et al., CA:a cancer journal for clinicians 63, 11-30 (2013)). Approximately 25% of patients have high-risk cytogenetics, which portends a median survival of less then 2 years (Boyd et al., Genes, chromosomes & cancer 50, 765-774 (2011); Shaughnessy et al., Blood 109, 2276-2284 (2007)). While recent strides have been made, regardless of cytogenetics, the disease is still considered incurable outside the immuno-therapeutic graft versus myeloma (GvM) effect of an allogeneic transplant. However, allogeneic transplants are limited by ineligibility and high rates of transplant-associated morbidity and mortality (Gahrton et al., The New England journal of medicine 325, 1267-1273 (1991)). Similar to the GvM effect, a potentially curative T cell effect may be achieved with minimal toxicity through autologous adoptive T cell therapy.

Myeloma is expected to be an ideal disease to test adoptive T cell therapy. First, allogeneic transplants demonstrate that the T cell can be a curative treatment, even with minimal or no concomitant chemotherapy such as after non-myeloablative transplants or post-transplantation donor lymphocyte infusions. Second, conditioning chemotherapy, possibly through the mechanism of depleting regulatory T cells (Tregs), enhances the efficacy of adoptive T cell therapy (Brentjens et al., Blood 118, 4817-4828 (2011) and Pegram et al., Blood 119, 4133-4141(2012)) as such, the immediate post-autologous transplant period could be an optimal time to administer T cells, and myeloma is one of the few diseases where autologous stem cell transplantation is the standard of care. Third, the immunomodulatory drug lenalidomide may improve CAR based therapy, as has been shown in mice (Bertilaccio et al., Blood 122, 4171 (2013)), and lenalidomide is commonly used to treat MM. Fourth, adoptive T cell therapy works best in bone marrow predominant disease such as ALL (Brentjens et al., Science translational medicine 5, 177ra138 (2013); Davila et al., Science translational medicine 6, 224ra225 (2014)), when compared to solid tumors or extra-medullary CLL (Brentjens et al. (2011)) and similar to ALL, myeloma is a disease of the bone marrow.

While there are various reasons to expect that adoptive T cell therapy may work well in MM, expanding adoptive T cell therapy to myeloma poses unique challenges. Unlike other B-cell malignancies, CD19 expression is seen in only 2% of myeloma patients (Bataille et al., Haematologica 91, 1234-1240 (2006)). Furthermore, unlike CD19, the common extracellular immunophenotypic markers in myeloma (CD138, CD38, and CD56) are all co-expressed on other essential cell types, and CARs to any of these targets could lead to unacceptable “off tumor, on target” toxicity (Brentjens et al. (2013)) which can be fatal even in targets where antibodies are well tolerated, as was the case with a HER2 targeted CAR (Morgan et al., Molecular therapy: the journal of the American Society of Gene Therapy 18, 843-851 (2010)). Accordingly, there are needs for novel therapeutic strategies to design CARs targeting antigens that are highly expressed in MM cells and limited expression in normal tissues for treating multiple myeloma, which strategies capable of inducing potent tumor eradication with minimal toxicity and immunogenicity.

### SUMMARY OF THE INVENTION

The presently disclosed subject matter generally provides chimeric antigen receptors (CARs) that specifically target Fc Receptor-like 5 (FcRL5), immunoresponsive cells comprising such CARs, and uses of these CARs and immunoresponsive cells for treating multiple myeloma.

The presently disclosed subject matter provides CARs. In one non-limiting example, the CAR comprises an extracellular antigen-binding domain, a transmembrane domain and an intracellular domain, where the extracellular antigen-binding domain specifically binds to FcRL5. In certain embodiments, the extracellular antigen-binding domain binds to domain 9 of FcRL5.

In certain non-limiting embodiments, the extracellular antigen-binding domain is a single-chain variable fragment (scFv). In certain embodiments, the extracellular antigen-binding domain is a murine scFv. In certain embodiments, the extracellular antigen-binding domain is a human scFv. In certain non-limiting embodiments, the extracellular antigen-binding domain is a Fab, which is optionally crosslinked. In certain non-limiting embodiments, the extracellular binding domain is a F(ab)₂. In certain non-limiting embodiments, any of the foregoing molecules can be comprised in a fusion protein with a heterologous sequence to form the extracellular antigen-binding domain. In certain embodiments, the extracellular antigen-binding domain specifically binds to FcRL5 with a binding affinity (K_{d}) of from about 1×10⁻¹¹ M to about 3×10⁻⁶ M, 1×10⁻¹⁰ M to about 3×10⁻⁶ M or 1×10⁻⁹ M to about 3×10⁻⁶ M. In certain embodiments, the extracellular antigen-binding domain specifically binds to domain 8 or 9 of FcRL5 with a K_{d} of from about 1×10⁻⁹ M to about 3×10⁻⁶ M.

In certain embodiments, the extracellular antigen-binding domain comprises a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917 and SEQ ID NO:921, wherein the extracellular antigen-binding domain binds to FcRL5.

In certain embodiments, the extracellular antigen-binding domain comprises a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915 and SEQ ID NO:919, wherein the extracellular antigen-binding domain binds to FcRL5.

In certain embodiments, the extracellular antigen-binding domain comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917 and SEQ ID NO:921; and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915 and SEQ ID NO:919, wherein the extracellular antigen-binding domain binds to FcRL5.

In certain embodiments, the extracellular antigen-binding domain comprises a light chain variable region comprising an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917, SEQ ID NO:921 and conservative modifications thereof. In certain embodiments, the extracellular antigen-binding domain comprises a heavy chain variable region comprising an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915, SEQ ID NO:919 and conservative modifications thereof.

In certain embodiments, the extracellular antigen-binding domain comprises a heavy chain variable region comprising amino acids having the sequence set forth in SEQ ID NO:915. In certain embodiments, the extracellular antigen-binding domain comprises a light chain variable region comprising amino acids having the sequence set forth in SEQ ID NO:917. In certain embodiments, the extracellular antigen-binding domain comprises a heavy chain variable region comprising amino acids having the sequence set forth in SEQ ID NO:919. In certain embodiments, the extracellular antigen-binding domain comprises a light chain variable region comprising amino acids having the sequence set forth in SEQ ID NO:921. In certain embodiments, the extracellular antigen-binding domain comprises a heavy chain variable region comprising amino acids having the sequence set forth in SEQ ID NO:144. In certain embodiments, the extracellular antigen-binding domain comprises a light chain variable region comprising amino acids having the sequence set forth in SEQ ID NO: 143. In certain embodiments, the extracellular antigen-binding domain comprises a heavy chain variable region comprising amino acids having the sequence set forth in SEQ ID NO:216. In certain embodiments, the extracellular antigen-binding domain comprises a light chain variable region comprising amino acids having the sequence set forth in SEQ ID NO:215. In certain embodiments, the extracellular antigen-binding domain comprises a heavy chain variable region comprising amino acids having the sequence set forth in SEQ ID NO:220. In certain embodiments, the extracellular antigen-binding domain comprises a light chain variable region comprising amino acids having the sequence set forth in SEQ ID NO:219. In certain embodiments, the extracellular antigen-binding domain comprises a heavy chain variable region comprising amino acids having the sequence set forth in SEQ ID NO:236. In certain embodiments, the extracellular antigen-binding domain comprises a light chain variable region comprising amino acids having the sequence set forth in SEQ ID NO:235. In certain embodiments, the extracellular antigen-binding domain comprises a heavy chain variable region comprising amino acids having the sequence set forth in SEQ ID NO:268. In certain embodiments, the extracellular antigen-binding domain comprises a light chain variable region comprising amino acids having the sequence set forth in SEQ ID NO:267. In certain embodiments, the extracellular antigen-binding domain comprises a heavy chain variable region comprising amino acids having the sequence set forth in SEQ ID NO:116. In certain embodiments, the extracellular antigen-binding domain comprises a light chain variable region comprising amino acids having the sequence set forth in SEQ ID NO:115. In certain embodiments, the extracellular antigen-binding domain comprises a heavy chain variable region comprising amino acids having the sequence set forth in SEQ ID NO:172. In certain embodiments, the extracellular antigen-binding domain comprises a light chain variable region comprising amino acids having the sequence set forth in SEQ ID NO:171. In certain embodiments, the extracellular antigen-binding domain comprises (a) a light chain variable region comprising an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917, SEQ ID NO:921 and conservative modifications thereof; and (b) a heavy chain variable region comprising an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915, SEQ ID NO:919 and conservative modifications thereof.

In certain embodiments, the extracellular antigen-binding domain comprises (a) a heavy chain variable region comprising amino acids having the sequence set forth in SEQ ID NO:915; and (b) a light chain variable region comprising amino acids having the sequence set forth in SEQ ID NO:917. In certain embodiments, the extracellular antigen-binding domain comprises extracellular antigen-binding domain comprises (a) a heavy chain variable region comprising amino acids having the sequence set forth in SEQ ID NO:919; and (b) a light chain variable region comprising amino acids having the sequence set forth in SEQ ID NO:921. In certain embodiments, the extracellular antigen-binding domain comprises (a) a heavy chain variable region comprising amino acids having the sequence set forth in SEQ ID NO:144, and (b) a light chain variable region comprising amino acids having the sequence set forth in SEQ ID NO:143. In certain embodiments, the extracellular antigen-binding domain comprises (a) a heavy chain variable region comprising amino acids having the sequence set forth in SEQ ID NO:216, and (b) a light chain variable region comprising amino acids having the sequence set forth in SEQ ID NO:215. In certain embodiments, the extracellular antigen-binding domain comprises (a) a heavy chain variable region comprising amino acids having the sequence set forth in SEQ ID NO:220, and (b) a light chain variable region comprising amino acids having the sequence set forth in SEQ ID NO:219. In certain embodiments, the extracellular antigen-binding domain comprises (a) a heavy chain variable region comprising amino acids having the sequence set forth in SEQ ID NO:236, and (b) a light chain variable region comprising amino acids having the sequence set forth in SEQ ID NO:235. In certain embodiments, the extracellular antigen-binding domain comprises (a) a heavy chain variable region comprising amino acids having the sequence set forth in SEQ ID NO:268, and (b) a light chain variable region comprising amino acids having the sequence set forth in SEQ ID NO:267. In certain embodiments, the extracellular antigen-binding domain comprises (a) a heavy chain variable region comprising amino acids having the sequence set forth in SEQ ID NO:116, and (b) a light chain variable region comprising amino acids having the sequence set forth in SEQ ID NO:115. In certain embodiments, the extracellular antigen-binding domain comprises (a) a heavy chain variable region comprising amino acids having the sequence set forth in SEQ ID NO:172, and (b) a light chain variable region comprising amino acids having the sequence set forth in SEQ ID NO:171.

In certain non-limiting embodiments, the extracellular antigen-binding domain comprises both of said heavy and light chains, optionally with a linker sequence, for example a linker peptide, between the heavy chain variable region and the light chain variable region. For example, in certain non-limiting embodiments, the extracellular antigen-binding domain comprises (a) a heavy chain variable region comprising amino acids having the sequence set forth in SEQ ID NO:915, and (b) a light chain variable region comprising amino acids having the sequence set forth in SEQ ID NO:917, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:919 and (b) a light chain variable region comprising amino acids having the sequence set forth in SEQ ID NO:92, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:144 and (b) a light chain variable region comprising amino acids having the sequence set forth in SEQ ID NO:143, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:216 and (b) a light chain variable region comprising amino acids having the sequence set forth in SEQ ID NO:215, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:220 and (b) a light chain variable region comprising amino acids having the sequence set forth in SEQ ID NO:219, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:236 and (b) a light chain variable region comprising amino acids having the sequence set forth in SEQ ID NO:235, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:268 and (b) a light chain variable region comprising amino acids having the sequence set forth in SEQ ID NO:267, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:116 and (b) a light chain variable region comprising amino acids having the sequence set forth in SEQ ID NO:115, optionally with (c) a linker sequence, for example a linker peptide, between the heavy chain variable region and the light chain variable region. In certain embodiments, the extracellular antigen-binding domain comprises (a) a heavy chain variable region comprising amino acids having the sequence set forth in SEQ ID NO:172 and (b) a light chain variable region comprising amino acids having the sequence set forth in SEQ ID NO:171, optionally with (c) a linker sequence, for example a linker peptide, between the heavy chain variable region and the light chain variable region.

In certain embodiments, the extracellular antigen-binding domain comprises (a) a heavy chain variable region CDR3 comprising an amino acid sequence selected from the group consisting of SEQ ID NOs: 311, 317, 323, 328, 334, 337, 342, 347, 351, 356, 362, 368, 374, 376, 380, 384, 389, 394, 396, 400, 405, 408, 412, 415, 422, 427, 432, 437, 442, 446, 451, 453, 456, 458, 459, 463, 464, 467, 473, 476, 482, 486, 489, 492, 494, 497, 502, 507, 512, 517, 522, 527, 529, 532, 536, 539, 543, 546, 550, 553, 555, 561, 567, 570, 574, 577, 578, 579, 584, 578, 587, 591, 925 and 931; and (b) a light chain variable region CDR3 comprising an amino acid sequence selected from the group consisting of SEQ ID NOs: 314, 320, 325, 331, 339, 345, 350, 353, 359, 365, 371, 377, 383, 386, 392, 395, 399, 402, 407, 410, 414, 418, 419, 424, 430, 435, 439, 443, 449, 452, 455, 457, 462, 465, 470, 479, 485, 488, 491, 493, 495, 499, 505, 509, 514, 519, 524, 528, 530, 531, 535, 541, 542, 545, 549, 554, 558, 564, 569, 573, 576, 581, 592, 928 and 934.

In certain embodiments, the extracellular antigen-binding domain comprises: (a) a heavy chain variable region CDR1 comprising an amino acid sequence selected from the group consisting of SEQ ID NOs: 309, 315, 321, 326, 332, 335, 340, 346, 354, 360, 366, 372, 378, 387, 393, 403, 411, 420, 425, 436, 440, 444, 471, 480, 500, 510, 515, 520, 525, 537, 551, 559, 565, 582, 589, 923 and 929; (b) a heavy chain variable region CDR2 comprising an amino acid sequence selected from the group consisting of SEQ ID NOs: 310, 316, 322, 327, 333, 336, 341, 355, 361, 367, 373, 379, 388, 404, 412, 421, 426, 431, 441, 445, 450, 466, 472, 475, 481, 496, 501, 506, 511, 516, 521, 526, 538, 552, 560, 566, 583, 590, 924 and 930; (c) a heavy chain variable region CDR3 comprising an amino acid sequence selected from the group consisting of SEQ ID NOs: 311, 317, 323, 328, 334, 337, 342, 347, 351, 356, 362, 368, 374, 376, 380, 384, 389, 394, 396, 400, 405, 408, 412, 415, 422, 427, 432, 437, 442, 446, 451, 453, 456, 458, 459, 463, 464, 467, 473, 476, 482, 486, 489, 492, 494, 497, 502, 507, 512, 517, 522, 527, 529, 532, 536, 539, 543, 546, 550, 553, 555, 561, 567, 570, 574, 577, 578, 579, 584, 578, 587, 591, 925 and 931; (d) a light chain variable region CDR1 comprising an amino acid sequence selected from the group consisting of SEQ ID NOs: 312, 318, 324, 329, 338, 343, 348, 352, 357, 363, 369, 381, 390, 397, 401, 406, 416, 423, 428, 433, 447, 460, 468, 474, 477, 483, 490, 498, 503, 508, 518, 533, 540, 544, 547, 556, 562, 568, 571, 580, 585, 588, 926 and 932; (e) a light chain variable region CDR2 comprising an amino acid sequence selected from the group consisting of SEQ ID NOs: 313, 319, 330, 344, 349, 358, 364, 370, 382, 385, 391, 398, 409, 417, 429, 434, 438, 448, 454, 461, 469, 478, 484, 487, 504, 513, 523, 534, 429, 448, 548, 557, 563, 572, 575, 586, 927 and 933; and (f) a light chain variable region CDR3 comprising an amino acid sequence selected from the group consisting of SEQ ID NOs: 314, 320, 325, 331, 339, 345, 350, 353, 359, 365, 371, 377, 383, 386, 392, 395, 399, 402, 407, 410, 414, 418, 419, 424, 430, 435, 439, 443, 449, 452, 455, 457, 462, 465, 470, 479, 485, 488, 491, 493, 495, 499, 505, 509, 514, 519, 524, 528, 530, 531, 535, 541, 542, 545, 549, 554, 558, 564, 569, 573, 576, 581, 592, 928 and 934.

In certain embodiments, the extracellular antigen-binding domain comprises (a) a heavy chain variable region CDR1 comprising amino acids having the sequence set forth in SEQ ID NO:923 or conservative modifications thereof, (b) a heavy chain variable region CDR2 comprising amino acids having the sequence set forth in SEQ ID NO:924 or conservative modifications thereof, and (c) a heavy chain variable region CDR3 comprising amino acids having the sequence set forth in SEQ ID NO:925 or conservative modifications thereof. In certain embodiments, the extracellular antigen-binding domain comprises (a) a light chain variable region CDR1 comprising amino acids having the sequence set forth in SEQ ID NO:926 or conservative modifications thereof, (b) a light chain variable region CDR2 comprising amino acids having the sequence set forth in SEQ ID NO:927 or conservative modifications thereof, and (c) a light chain variable region CDR3 comprising amino acids having the sequence set forth in SEQ ID NO:928 or conservative modifications thereof.

In certain embodiments, the extracellular antigen-binding domain comprises (a) a heavy chain variable region CDR1 comprising amino acids having the sequence set forth in SEQ ID NO:929 or conservative modifications thereof, (b) a heavy chain variable region CDR2 comprising amino acids having the sequence set forth in SEQ ID NO:930 or conservative modifications thereof, and (c) a heavy chain variable region CDR3 comprising amino acids having the sequence set forth in SEQ ID NO:931 or conservative modifications thereof. In certain embodiments, the extracellular antigen-binding domain comprises (a) a light chain variable region CDR1 comprising amino acids having the sequence set forth in SEQ ID NO:932 or conservative modifications thereof, (b) a light chain variable region CDR2 comprising amino acids having the sequence set forth in SEQ ID NO:933 or conservative modifications thereof, and (c) a light chain variable region CDR3 comprising amino acids having the sequence set forth in SEQ ID NO:934 or conservative modifications thereof.

In certain embodiments, the extracellular antigen-binding domain comprises: (a) a heavy chain variable region CDR1 comprising amino acids having the sequence set forth in SEQ ID NO:923 or conservative modifications thereof, (b) a heavy chain variable region CDR2 comprising amino acids having the sequence set forth in SEQ ID NO:924 or conservative modifications thereof, (c) a heavy chain variable region CDR3 comprising amino acids having the sequence set forth in SEQ ID NO:925 or conservative modifications thereof, (d) a light chain variable region CDR1 comprising amino acids having the sequence set forth in SEQ ID NO:926 or conservative modifications thereof, (e) a light chain variable region CDR2 comprising amino acids having the sequence set forth in SEQ ID NO:927 or conservative modifications thereof, and (f) a light chain variable region CDR3 comprising amino acids having the sequence set forth in SEQ ID NO:928 or conservative modifications thereof.

In certain embodiments, the extracellular antigen-binding domain comprises: (a) a heavy chain variable region CDR1 comprising amino acids having the sequence set forth in SEQ ID NO:929 or conservative modifications thereof, (b) a heavy chain variable region CDR2 comprising amino acids having the sequence set forth in SEQ ID NO:930 or conservative modifications thereof, (c) a heavy chain variable region CDR3 comprising amino acids having the sequence set forth in SEQ ID NO:931 or conservative modifications thereof, (d) a light chain variable region CDR1 comprising amino acids having the sequence set forth in SEQ ID NO:932 or conservative modifications thereof, (e) a light chain variable region CDR2 comprising amino acids having the sequence set forth in SEQ ID NO:933 or conservative modifications thereof, and (f) a light chain variable region CDR3 comprising amino acids having the sequence set forth in SEQ ID NO:934 or conservative modifications thereof.

In certain embodiments, the extracellular antigen-binding domain comprises: (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; (c) a heavy chain variable region CDR3 comprising the amino acid sequence of SEQ ID NO:463 or conservative modifications thereof; (d) a light chain variable region CDR1 comprising the amino acid sequence of SEQ ID NO:318 or conservative modifications thereof; (e) a light chain variable region CDR2 comprising the amino acid sequence of SEQ ID NO:319 or conservative modifications thereof; and (f) a light chain variable region CDR3 comprising the amino acid sequence of SEQ ID NO:419 or conservative modifications thereof.

In certain embodiments, the extracellular antigen-binding domain comprises: (a) a heavy chain variable region CDR1 comprising the amino acid sequence of SEQ ID NO:515 or conservative modifications thereof; (b) a heavy chain variable region CDR2 comprising the amino acid sequence of SEQ ID NO:516 or conservative modifications thereof; (c) a heavy chain variable region CDR3 comprising the amino acid sequence of SEQ ID NO:517 or conservative modifications thereof; (d) a light chain variable region CDR1 comprising the amino acid sequence of SEQ ID NO:318 or conservative modifications thereof; (e) a light chain variable region CDR2 comprising the amino acid sequence of SEQ ID NO:319 or conservative modifications thereof; and (f) a light chain variable region CDR3 comprising the amino acid sequence of SEQ ID NO:531 or conservative modifications thereof.

In certain embodiments, the extracellular antigen-binding comprises: (a) a heavy chain variable region CDR1 comprising the amino acid sequence of SEQ ID NO:403 or conservative modifications thereof; (b) a heavy chain variable region CDR2 comprising the amino acid sequence of SEQ ID NO:404 or conservative modifications thereof; (c) a heavy chain variable region CDR3 comprising the amino acid sequence of SEQ ID NO:532 or conservative modifications thereof; (d) a light chain variable region CDR1 comprising the amino acid sequence of SEQ ID NO:533 or conservative modifications thereof; (e) a light chain variable region CDR2 comprising the amino acid sequence of SEQ ID NO:534 or conservative modifications thereof; and (f) a light chain variable region CDR3 comprising the amino acid sequence of SEQ ID NO:535.

In certain embodiments, the extracellular antigen-binding domain comprises: (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; (c) a heavy chain variable region CDR3 comprising the amino acid sequence of SEQ ID NO:543 or conservative modifications thereof; (d) a light chain variable region CDR1 comprising the amino acid sequence of SEQ ID NO:544 or conservative modifications thereof; (e) a light chain variable region CDR2 comprising the amino acid sequence of SEQ ID NO:448 or conservative modifications thereof; and (f) a light chain variable region CDR3 comprising the amino acid sequence of SEQ ID NO:545 or conservative modifications thereof.

In certain embodiments, the extracellular antigen-binding domain comprises: (a) a heavy chain variable region CDR1 comprising the amino acid sequence of SEQ ID NO:372 or conservative modifications thereof; (b) a heavy chain variable region CDR2 comprising the amino acid sequence of SEQ ID NO:475 or conservative modifications thereof; (c) a heavy chain variable region CDR3 comprising the amino acid sequence of SEQ ID NO:570 or conservative modifications thereof; (d) a light chain variable region CDR1 comprising the amino acid sequence of SEQ ID NO:571 thereof; (e) a light chain variable region CDR2 comprising the amino acid sequence of SEQ ID NO:572 or conservative modifications thereof; and (f) a light chain variable region CDR3 comprising the amino acid sequence of SEQ ID NO:573 or conservative modifications thereof.

In certain embodiments, the extracellular antigen-binding domain comprises: (a) a heavy chain variable region CDR1 comprising the amino acid sequence of SEQ ID NO:440 or conservative modifications thereof; (b) a heavy chain variable region CDR2 comprising the amino acid sequence of SEQ ID NO:441 or conservative modifications thereof; (c) a heavy chain variable region CDR3 comprising the amino acid sequence of SEQ ID NO:442 or conservative modifications thereof; (d) a light chain variable region CDR1 comprising the amino acid sequence of SEQ ID NO:329 or conservative modifications thereof; (e) a light chain variable region CDR2 comprising the amino acid sequence of SEQ ID NO:330 or conservative modifications thereof; and (f) a light chain variable region CDR3 comprising the amino acid sequence of SEQ ID NO:443 or conservative modifications thereof.

In certain embodiments, the extracellular antigen-binding domain comprises: (a) a heavy chain variable region CDR1 comprising the amino acid sequence of SEQ ID NO:309 or conservative modifications thereof; (b) a heavy chain variable region CDR2 comprising the amino acid sequence of SEQ ID NO:310 or conservative modifications thereof; (c) a heavy chain variable region CDR3 comprising the amino acid sequence of SEQ ID NO:489 or conservative modifications thereof; (d) a light chain variable region CDR1 comprising the amino acid sequence of SEQ ID NO:490 or conservative modifications thereof; (e) a light chain variable region CDR2 comprising the amino acid sequence of SEQ ID NO:313 or conservative modifications thereof; and (f) a light chain variable region CDR3 comprising the amino acid sequence of SEQ ID NO:491 or conservative modifications thereof.

In certain non-limiting embodiments, the extracellular antigen-binding domain comprises amino acids having the sequence set forth in SEQ ID NO:664 or conservative modifications thereof. In certain embodiments, the extracellular antigen-binding domain comprises amino acids having the sequence set forth in SEQ ID NO:700 or conservative modifications thereof. In certain embodiments, the extracellular antigen-binding domain comprises amino acids having the sequence set forth in SEQ ID NO:702 or conservative modifications thereof. In certain embodiments, the extracellular antigen-binding domain comprises amino acids having the sequence set forth in SEQ ID NO:710 or conservative modifications thereof. In certain embodiments, the extracellular antigen-binding domain comprises amino acids having the sequence set forth in SEQ ID NO:726 or conservative modifications thereof. In certain embodiments, the extracellular antigen-binding domain comprises amino acids having the sequence set forth in SEQ ID NO:650 or conservative modifications thereof. In certain embodiments, the extracellular antigen-binding domain comprises amino acids having the sequence set forth in SEQ ID NO:678 or conservative modifications thereof.

In certain non-limiting embodiments, the extracellular antigen-binding domain comprises (a) a heavy chain variable region CDR1 comprising amino acids having the sequence set forth in SEQ ID NO:923 or conservative modifications thereof, a heavy chain variable region CDR2 comprising amino acids having the sequence set forth in SEQ ID NO:924 or conservative modifications thereof, and a heavy chain variable region CDR3 comprising amino acids having the sequence set forth in SEQ ID NO:925 or conservative modifications thereof, and (ii) a light chain variable region CDR1 comprising amino acids having the sequence set forth in SEQ ID NO:926 or conservative modifications thereof, a light chain variable region CDR2 comprising amino acids having the sequence set forth in SEQ ID NO:927 or conservative modifications thereof, and a light chain variable region CDR3 comprising amino acids having the sequence set forth in SEQ ID NO:928 or conservative modifications thereof, optionally with (iii) a linker sequence, for example a linker peptide, between the heavy chain variable region and the light chain variable region.

In another non-limiting embodiment, the extracellular antigen-binding domain comprises (i) a heavy chain variable region CDR1 comprising amino acids having the sequence set forth in SEQ ID NO:929 or conservative modifications thereof, a heavy chain variable region CDR2 comprising amino acids having the sequence set forth in SEQ ID NO:930 or conservative modifications thereof, and a heavy chain variable region CDR3 comprising amino acids having the sequence set forth in SEQ ID NO:931 or conservative modifications thereof, and (ii) a light chain variable region CDR1 comprising amino acids having the sequence set forth in SEQ ID NO:932 or conservative modifications thereof, a light chain variable region CDR2 comprising amino acids having the sequence set forth in SEQ ID NO:933 or conservative modifications thereof, and a light chain variable region CDR3 comprising amino acids having the sequence set forth in SEQ ID NO:934 or conservative modifications thereof, optionally with (iii) a linker sequence, for example a linker peptide, between the heavy chain variable region and the light chain variable region.

In certain embodiments, the extracellular antigen-binding domain comprises: (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; (c) a heavy chain variable region CDR3 comprising the amino acid sequence of SEQ ID NO:463 or conservative modifications thereof; (d) a light chain variable region CDR1 comprising the amino acid sequence of SEQ ID NO:318 or conservative modifications thereof; (e) a light chain variable region CDR2 comprising the amino acid sequence of SEQ ID NO:319 or conservative modifications thereof; and (f) a light chain variable region CDR3 comprising the amino acid sequence of SEQ ID NO:419 or conservative modifications thereof, optionally with (g) a linker sequence, for example a linker peptide, between the heavy chain variable region and the light chain variable region.

In certain embodiments, the extracellular antigen-binding domain comprises: (a) a heavy chain variable region CDR1 comprising the amino acid sequence of SEQ ID NO:515 or conservative modifications thereof; (b) a heavy chain variable region CDR2 comprising the amino acid sequence of SEQ ID NO:516 or conservative modifications thereof; (c) a heavy chain variable region CDR3 comprising the amino acid sequence of SEQ ID NO:517 or conservative modifications thereof; (d) a light chain variable region CDR1 comprising the amino acid sequence of SEQ ID NO:318 or conservative modifications thereof; (e) a light chain variable region CDR2 comprising the amino acid sequence of SEQ ID NO:319 or conservative modifications thereof; and (f) a light chain variable region CDR3 comprising the amino acid sequence of SEQ ID NO:531 or conservative modifications thereof, optionally with (g) a linker sequence, for example a linker peptide, between the heavy chain variable region and the light chain variable region.

In certain embodiments, the extracellular antigen-binding comprises: (a) a heavy chain variable region CDR1 comprising the amino acid sequence of SEQ ID NO:403 or conservative modifications thereof; (b) a heavy chain variable region CDR2 comprising the amino acid sequence of SEQ ID NO:404 or conservative modifications thereof; (c) a heavy chain variable region CDR3 comprising the amino acid sequence of SEQ ID NO:532 or conservative modifications thereof; (d) a light chain variable region CDR1 comprising the amino acid sequence of SEQ ID NO:533 or conservative modifications thereof; (e) a light chain variable region CDR2 comprising the amino acid sequence of SEQ ID NO:534 or conservative modifications thereof; and (f) a light chain variable region CDR3 comprising the amino acid sequence of SEQ ID NO:535, optionally with (g) a linker sequence, for example a linker peptide, between the heavy chain variable region and the light chain variable region. In certain embodiments, the extracellular antigen-binding domain comprises: (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; (c) a heavy chain variable region CDR3 comprising the amino acid sequence of SEQ ID NO:543 or conservative modifications thereof; (d) a light chain variable region CDR1 comprising the amino acid sequence of SEQ ID NO:544 or conservative modifications thereof; (e) a light chain variable region CDR2 comprising the amino acid sequence of SEQ ID NO:448 or conservative modifications thereof; and (f) a light chain variable region CDR3 comprising the amino acid sequence of SEQ ID NO:545 or conservative modifications thereof, optionally with (g) a linker sequence, for example a linker peptide, between the heavy chain variable region and the light chain variable region.

In certain embodiments, the extracellular antigen-binding domain comprises: (a) a heavy chain variable region CDR1 comprising the amino acid sequence of SEQ ID NO:372 or conservative modifications thereof; (b) a heavy chain variable region CDR2 comprising the amino acid sequence of SEQ ID NO:475 or conservative modifications thereof; (c) a heavy chain variable region CDR3 comprising the amino acid sequence of SEQ ID NO:570 or conservative modifications thereof; (d) a light chain variable region CDR1 comprising the amino acid sequence of SEQ ID NO:571 or conservative modifications thereof; (e) a light chain variable region CDR2 comprising the amino acid sequence of SEQ ID NO:572 or conservative modifications thereof; and (f) a light chain variable region CDR3 comprising the amino acid sequence of SEQ ID NO:573 or conservative modifications thereof, optionally with (g) a linker sequence, for example a linker peptide, between the heavy chain variable region and the light chain variable region.

In certain embodiments, the extracellular antigen-binding domain comprises: (a) a heavy chain variable region CDR1 comprising the amino acid sequence of SEQ ID NO:440 or conservative modifications thereof; (b) a heavy chain variable region CDR2 comprising the amino acid sequence of SEQ ID NO:441 or conservative modifications thereof; (c) a heavy chain variable region CDR3 comprising the amino acid sequence of SEQ ID NO:442 or conservative modifications thereof; (d) a light chain variable region CDR1 comprising the amino acid sequence of SEQ ID NO:329 or conservative modifications thereof; (e) a light chain variable region CDR2 comprising the amino acid sequence of SEQ ID NO:330 or conservative modifications thereof; and (f) a light chain variable region CDR3 comprising the amino acid sequence of SEQ ID NO:443 or conservative modifications thereof, optionally with (g) a linker sequence, for example a linker peptide, between the heavy chain variable region and the light chain variable region.

In certain embodiments, the extracellular antigen-binding domain comprises: (a) a heavy chain variable region CDR1 comprising the amino acid sequence of SEQ ID NO:309 or conservative modifications thereof; (b) a heavy chain variable region CDR2 comprising the amino acid sequence of SEQ ID NO:310 or conservative modifications thereof; (c) a heavy chain variable region CDR3 comprising the amino acid sequence of SEQ ID NO:489 or conservative modifications thereof; (d) a light chain variable region CDR1 comprising the amino acid sequence of SEQ ID NO:490 or conservative modifications thereof; (e) a light chain variable region CDR2 comprising the amino acid sequence of SEQ ID NO:313 or conservative modifications thereof; and (f) a light chain variable region CDR3 comprising the amino acid sequence of SEQ ID NO:491 or conservative modifications thereof, optionally with (g) a linker sequence, for example a linker peptide, between the heavy chain variable region and the light chain variable region.

In certain embodiments, the linker peptide comprises an amino acid sequence selected from the group consisting of SEQ ID NO:307 and SEQ ID NO:897.

In certain embodiments, the extracellular antigen-binding domain binds to FcRL5 comprising the amino acid sequence set forth in SEQ ID NO:899. In certain embodiments, the extracellular antigen-binding domain binds to an epitope comprising the amino acid sequence set forth in SEQ ID NO:964. In certain embodiments, the extracellular antigen-binding domain binds to an epitope comprising the amino acid sequence set forth in SEQ ID NO:965.

In accordance with the presently disclosed subject matter, the extracellular antigen-binding domain is covalently joined to a transmembrane domain. The extracellular antigen-binding domain can comprise a signal peptide that is covalently joined to the 5′ terminus of the extracellular antigen-binding domain. In certain embodiments, the transmembrane domain of the CAR comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof. In a non-limiting embodiment, the transmembrane domain comprises a CD8 polypeptide. In certain embodiments, the transmembrane domain comprises a CD28 polypeptide.

In accordance with the presently disclosed subject matter, in certain embodiments, the intracellular domain comprises a CD3ζ polypeptide. In certain embodiments, the intracellular domain further comprises at least one signaling region. In certain embodiments, the at least one signaling region comprises a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof. In certain embodiments, the signaling region is a co-stimulatory signaling region. In certain embodiments, the co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide. In certain non-limiting embodiments, the transmembrane domain comprises a CD28 polypeptide, the intracellular domain comprises a CD3ζ polypeptide, and the co-stimulatory signaling domain comprises a CD28 polypeptide. In certain non-limiting embodiments, the transmembrane domain comprises a CD8 polypeptide, the intracellular domain comprises a CD3ζ polypeptide, and the co-stimulatory signaling domain comprises a 4-1BB polypeptide.

In certain embodiments, the CAR is recombinantly expressed. The CAR can be expressed from a vector. In certain embodiments, the vector is a γ-retroviral rector.

The presently disclosed subject matter also provides isolated immunoresponsive cells comprising the above-described CARs. In certain embodiments, the isolated immunoresponsive cell is transduced with the CAR, for example, the CAR is constitutively expressed on the surface of the immunoresponsive cell. In certain embodiments, the isolated immunoresponsive cell is further transduced with at least one co-stimulatory ligand such that the immunoresponsive cell expresses the at least one co-stimulatory ligand. In certain embodiments, the at least one co-stimulatory ligand is selected from the group consisting of 4-1BBL, CD80, CD86, CD70, OX40L, CD48, TNFRSF14, and combinations thereof. In certain embodiments, the isolated immunoresponsive cell is further transduced with at least one cytokine such that the immunoresponsive cell secrets the at least one cytokine. In certain embodiments, the at least one cytokine is selected from the group consisting of IL-2, IL-3, IL-6, IL-7, IL-11, IL-12, IL-15, IL-17, IL-21, and combinations thereof. In certain embodiments, the isolated immunoresponsive cell is selected from the group consisting of a T cell, a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated. In certain embodiments, the immunoresponsive cell is a T cell. The presently disclosed subject matter further provides nucleic acid molecules encoding the presently disclosed CARs, vectors comprising the nucleic acid molecules, and host cells expressing such nucleic acid molecules. In certain embodiments, the nucleic acid molecule comprises nucleic acids having the sequence set forth in SEQ ID NO:951. In certain embodiments, the nucleic acid molecule comprises nucleic acids having the sequence set forth in SEQ ID NO:952. In certain embodiments, the vector is a γ-retroviral vector. In certain embodiments, the host cell is a T cell. Furthermore, the presently disclosed subject matter provides methods of using the above-described immunoresponsive cell for reducing tumor burden in a subject. For example, the presently disclosed subject matter provides methods of reducing tumor burden in a subject, where the method comprises administering an effective amount of the presently disclosed immunoresponsive cell to the subject, thereby inducing tumor cell death in the subject. In certain embodiments, the method reduces the number of tumor cells. In another embodiment, the method reduces the tumor size. In yet another embodiment, the method eradicates the tumor in the subject. In certain embodiments, the tumor is selected from the group consisting of multiple myeloma, Non-Hodgkin Lymphoma (especially Mantle Cell), Hodgkin Lymphoma, Chronic Lymphocytic Leukemia (CLL), Acute lymphocytic leukemia (ALL), Hairy Cell Leukemia, Burketts Lymphoma, and Waldenstrom's Macroglobulinemia. In certain embodiments, the tumor is multiple myeloma. In certain embodiments, the subject is a human. In certain embodiments, the immunoresponsive cell is a T cell.

Furthermore, the presently disclosed subject matter provides methods of using the above-described immunoresponsive cell for increasing or lengthening survival of a subject having neoplasia. For example, the presently disclosed subject matter provides methods of increasing or lengthening survival of a subject having neoplasia, where the method comprises administering an effective amount of the presently disclosed immunoresponsive cell to the subject, thereby increasing or lengthening survival of the subject. In certain embodiments, the neoplasia is selected from the group consisting of multiple myeloma, Non-Hodgkin Lymphoma (especially Mantle Cell), Hodgkin Lymphoma, Chronic Lymphocytic Leukemia (CLL), Acute lymphocytic leukemia (ALL), Hairy Cell Leukemia, Burketts Lymphoma, and Waldenstrom's Macroglobulinemia. In certain embodiments, the neoplasia is multiple myeloma. In certain embodiments, the method reduces or eradicates tumor burden in the subject.

The presently disclosed subject matter also provides methods for producing an immunoresponsive cell that binds to Fc Receptor-like 5 (FcRL5), e.g., domain 9 of FcRL5. In one non-limiting example, the method comprises introducing into the immunoresponsive cell a nucleic acid sequence that encodes a chimeric antigen receptor (CAR), which comprises an extracellular antigen-binding domain, a transmembrane domain and an intracellular domain, wherein the extracellular antigen-binding domain specifically binds to Fc Receptor-like 5 (FcRL5). In a specific non-limiting embodiment, the extracellular antigen-binding domain is an scFv.

The presently disclosed subject matter further provides pharmaceutical compositions comprising an effective amount of the presently disclosed immunoresponsive cells and a pharmaceutically acceptable excipient. In certain embodiments, the pharmaceutical compositions are for treating a neoplasia. In certain embodiments, the neoplasia is selected from the group consisting of multiple myeloma, Non-Hodgkin Lymphoma (especially Mantle Cell), Hodgkin Lymphoma, Chronic Lymphocytic Leukemia (CLL), Acute lymphocytic leukemia (ALL), Hairy Cell Leukemia, Burketts Lymphoma, and Waldenstrom's Macroglobulinemia. In certain embodiments, the neoplasia is multiple myeloma.

The presently disclosed subject matter further provides kits for treating a neoplasia, comprising the presently disclosed immunoresponsive cells. In certain embodiments, the kit further include written instructions for using the immunoresponsive cell for treating a neoplasia. In certain embodiments, the neoplasia is selected from the group consisting of multiple myeloma, Non-Hodgkin Lymphoma (especially Mantle Cell), Hodgkin Lymphoma, Chronic Lymphocytic Leukemia (CLL), Acute lymphocytic leukemia (ALL), Hairy Cell Leukemia, Burketts Lymphoma, and Waldenstrom's Macroglobulinemia. In certain embodiments, the neoplasia is multiple myeloma.

### BRIEF DESCRIPTION OF THE FIGURES

The following Detailed Description, given by way of example, but not intended to limit the invention to specific embodiments described, may be understood in conjunction with the accompanying drawings.

FIGS. 1A-1D depict the FcRL5 expression in various normal tissues and human cancer cell lines.

FIG. 2 depicts the screening of anti-FcRL5 scFvs using 3T3 cells expressing FcRL5 or FcRL1, 2, 3, 4 or 6.

FIGS. 3A-3E. (FIG. 3A) A representation of the domains of FcRL5 and the soluble, glycosylphosphatidylinositol (GPI)-anchored and transmembrane forms of FcRL5. (FIG. 3B) A representation of the vector used to express a mutated form of FcRL5 that lacks domain 9 (also referred to herein as FcRL5Δdom9). (FIG. 3C) The nucleotide sequences of full length FcRL5 and the form of FcRL5 that lacks domain 9. (FIGS. 3D and 3E) A representation of the differences in the nucleotide sequences of full length FcRL5 and the mutated form of FcRL5 in which domain 9 is deleted (referred to herein as “FcRL5Δdom9”).

FIG. 4 depicts the screening of anti-FcRL5 scFv ET200-39 on 3T3 cells expressing FcRL5Δdom9.

FIG. 5 depicts the screening of anti-FcRL5 scFv ET200-104 on 3T3 cells expressing FcRL5Δdom9.

FIGS. 6A and 6B depict the screening of anti-FcRL5 scFv ET200-105 on 3T3 cells expressing FcRL5Δdom9.

FIG. 7 depicts the screening of anti-FcRL5 scFv ET200-109 on 3T3 cells expressing FcRL5Δdom9.

FIG. 8 depicts the screening of anti-FcRL5 scFv ET200-117 on 3T3 cells expressing FcRL5Δdom9.

FIGS. 9A-9B. Schematics of chimeric antigen receptor targeting FcRL5 in accordance with non-limiting embodiments of the presently disclosed subject matter. (FIG. 9A) Schematic of FcRL5-targeted CAR with CD28 co-stimulatory domain and CD3zeta. (FIG. 9B) Schematic of FcRL5-targeted CAR with 4-1BB co-stimulatory domain and CD3zeta.

FIG. 10 depicts the vector map of a chimeric antigen receptor targeting FcRL5 using scFV ET200-31 in accordance with one non-limiting embodiment of the presently disclosed subject matter.

FIG. 11 depicts the vector map of a chimeric antigen receptor targeting FcRL5 using scFV ET200-39 in accordance with one non-limiting embodiment of the presently disclosed subject matter.

FIG. 12 depicts the vector map of a chimeric antigen receptor targeting FcRL5 using scFV ET200-69 in accordance with one non-limiting embodiment of the presently disclosed subject matter.

FIG. 13 depicts the vector map of a chimeric antigen receptor targeting FcRL5 using scFV ET200-104 in accordance with one non-limiting embodiment of the presently disclosed subject matter.

FIG. 14 depicts the vector map of a chimeric antigen receptor targeting FcRL5 using scFV ET200-105 in accordance with one non-limiting embodiment of the presently disclosed subject matter.

FIG. 15 depicts the vector map of a chimeric antigen receptor targeting FcRL5 using scFV ET200-109 in accordance with one non-limiting embodiment of the presently disclosed subject matter.

FIG. 16 depicts the vector map of a chimeric antigen receptor targeting FcRL5 using scFV ET200-117 in accordance with one non-limiting embodiment of the presently disclosed subject matter.

FIG. 17 depicts the expression of FcRL5-targeted chimeric antigen receptors on the surface of transduced T cells.

FIG. 18 depicts the cytotoxicity of FcRL5-targeted chimeric antigen receptor T cells for FcRL5-expressing cells.

FIG. 19 depicts the induction of cytokine secretion of FcRL5-targeted chimeric antigen receptor T cells.

FIG. 20 depicts the proliferation of FcRL5-targeted chimeric antigen receptor T cells upon antigen stimulation.

FIG. 21 illustrates the CLIPS technology. The CLIPS reaction takes place between bromo groups of the CLIPS scaffold and thiol sidechains of cysteines. The reaction is fast and specific under mild conditions. Using this elegant chemistry, native protein sequences are transformed into CLIPS constructs with a range of structures. From left to right: two different single T2 loops, T3 double loop, conjugated T2+ T3 loops, stabilized beta sheet, and stabilized alpha helix (Timmerman et al., J. Mol. Recognit. 2007; 20: 283-29).

FIG. 22 illustrates combinatorial clips library screening. The target protein (left) containing a discontinuous conformational epitope is converted into a matrix library (middle). Combinatorial peptides are synthesized on a proprietary minicard and chemically converted into spatially defined CLIPS constructs (right).

FIG. 23 depicts T3 looped CLIPS™ construct.

FIGS. 24A-24D illustrate heat map technology. (FIG. 24A) Table of combined peptides, with two sub-sequences indicated as “Loop 1” and “Loop 2.” (FIG. 24B) Data from A displayed as a matrix. (FIG. 24C) Color bar indication of the heat map representation. (FIG. 24D) Heat map visualization of data from A.

FIGS. 25A-25D show heatmap analysis of data recorded for Herceptin.

FIGS. 26A-26D show heatmap analysis of data recorded for ET200-104.

FIG. 27 illustrates a 3D model of amino acid residues 380-731 of FcRL55 with peptide stretch₆₅₇SRPILTFRAPR₆₆₇ highlighted.

### DETAILED DESCRIPTION OF THE INVENTION

The presently disclosed subject matter generally provides FcRL5-targeted chimeric antigen receptors (CARs). In one non-limiting example, the CAR comprises an extracellular antigen-binding domain, a transmembrane domain and an intracellular domain, where the extracellular antigen-binding domain specifically binds to FcRL5. In certain embodiments, the extracellular antigen-binding domain specifically binds to domain 7, 8 or 9 of FcRL5. The presently disclosed subject matter also provides immunoresponsive cells (e.g., T cell, a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated) expressing the FcRL5-targeted CARs, and methods of using such immunoresponsive cells for treating a tumor, e.g., multiple myeloma.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker. In a non-limiting example, the linker comprises amino acids having the sequence set forth in SEQ ID NO:897 as provided below.

GGGGSGGGGSGGGGS [SEQ ID NO: 897]. In certain embodiments, the nucleic acid sequence encoding the amino acid sequence of SEQ ID NO:897 is set forth in SEQ ID NO:898, which is provided below:

[SEQ ID NO: 898]
GGTGGAGGTGGATCAGGTGGAGGTGGATCTGGTGGAGGTGGATCT.

In another non-limiting example, the linker comprises amino acids having the sequence set forth in SEQ ID NO:307 as provided below: SRGGGGSGGGGSGGGGSLEMA [SEQ ID NO: 307] In certain embodiments, the nucleic acid sequence encoding the amino acid sequence of SEQ ID NO:307 is set forth in SEQ ID NO:305, which is provided below:

[SEQ ID NO: 305]
TCTAGAGGTGGTGGTGGTAGCGGCGGCGGCGGCTCTGGTGGTGGTGGAT
CCCTCGAGATGGCC.

Despite removal of the constant regions and the introduction of a linker, scFv proteins retain the specificity of the original immunoglobulin. Single chain Fv polypeptide antibodies can be expressed from a nucleic acid comprising V_{H}—and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 August 12; Shieh et al., J Imunol2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, i.e., recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, comprising use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

As used herein, the term “analog” refers to a structurally related polypeptide or nucleic acid molecule having the function of a reference polypeptide or nucleic acid molecule.

As used herein, the term “ligand” refers to a molecule that binds to a receptor. In particular, the ligand binds a receptor on another cell, allowing for cell-to-cell recognition and/or interaction.

As used herein, the term “disease” refers to any condition or disorder that damages or interferes with the normal function of a cell, tissue, or organ. Examples of diseases include neoplasia or pathogen infection of cell.

As used herein, the term “effective amount” refers to an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasia.

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which naturally includes a polypeptide of the invention.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### H. Fc Receptor-Like 5 (FcRL5)

Fc Receptor-Like 5 (FcRL5) (also known as “CD307e” or “IRTA2”) is a rational target for treating multiple myeloma as it is expressed on B cells and plasma cells. FcRL5 binds to the Fc portion of IgG and contributes to B cell receptor signaling and B cell proliferation (Franco et al., Journal of immunology 190, 5739-5746 (2013); Dement-Brown et al., Journal of leukocyte biology 91, 59-67 (2012). FcRL5 was found to be an alternative to CD138 as a FACS marker for malignant plasma cells from fresh or frozen patient samples with a mean relative MFI between 10-55 (n=23) (Ise et al., Leukemia 21, 169-174 (2007)). Another study confirmed cell surface expression of FcRL5 by FACS on primary patient samples from most chronic lymphocytic leukemia (CLL), and mantle cell lymphoma cases, and all multiple myeloma (MM) (n=8) cases tested (Ise et al. (2007)). A third group found high surface staining on plasma cells from normal bone marrows (n=7), in MGUS (n=16), and in MM (n=16), (MFI similar in all three groups, ˜1000 fold increase compared to isotype control) (Elkins et al., Molecular cancer therapeutics 11, 2222-2232 (2012)). FcRL5 is on 1q21 and has been found to be involved in 1q21 abnormalities in B cell malignancies (Hatzivassiliou et al., Immunity 14, 277-289 (2001)). Amplification of 1q21 is found in 48% of MM patients at diagnosis and 67% of patients at relapse, and correlates with a worse prognosis (An et al., Haematologica 99, 353-359 (2014)). An antibody-drug conjugate targeting FcRL5 was effective in treating an in vivo murine model of MM (Elkins et al. (2012)).

Non-limiting examples of human FcRL5 amino acid sequences can be found under GenBank Protein Accession Nos: AAI01070.1; XP_011508332.1; XP_011508334.1; XP_011508333.1; XP_011508332.1; and NP_001182317.1.

In certain non-limiting embodiments, FcRL5 is human FcRL5 having the amino acid sequence set forth in SEQ ID NO:899, or fragments thereof. SEQ ID NO:899 is provided below:

[SEQ ID NO: 899]
MLLWVILLVLAPVSGQFARTPRPIIFLQPPWTTVFQGERVTLTCKGFRF
YSPQKTKWYHRYLGKEILRETPDNILEVQESGEYRCQAQGSPLSSPVHL
DFSSASLILQAPLSVFEGDSVVLRCRAKAEVTLNNTIYKNDNVLAFLNK
RTDFHIPHACLKDNGAYRCTGYKESCCPVSSNTVKIQVQEPFTRPVLRA
SSFQPISGNPVTLTCETQLSLERSDVPLRFRFFRDDQTLGLGWSLSPNF
QITAMWSKDSGFYWCKAATMPHSIISDSPRSWIQVQIPASHPVLTLSPE
KALNFEGTKVTLHCETQEDSLRTLYRFYHEGVPLRHKSVRCERGASISF
SLTTENSGNYYCTADNGLGAKPSKAVSLSVTVPVSHPVLNLSSPEDLIF
EGAKVTLHCEAQRGSLPILYQFHHEDAALERRSANSAGGVAISFSLTAE
HSGNYYCTADNGFGPQRSKAVSLSITVPVSHPVLTLSSAEALTFEGATV
TLHCEVQRGSPQILYQFYHEDMPLWSSSTPSVGRVSFSFSLTEGHSGNY
YCTADNGFGPQRSEVVSLFVTVPVSRPILTLRVPRAQAVVGDLLELHCE
APRGSPPILYWFYHEDVTLGSSSAPSGGEASFNLSLTAEHSGNYSCEAN
NGLVAQHSDTISLSVIVPVSRPILTFRAPRAQAVVGDLLELHCEALRGS
SPILYWFYHEDVTLGKISAPSGGGASFNLSLTTEHSGIYSCEADNGLEA
QRSEMVTLKVAVPVSRPVLTLRAPGTHAAVGDLLELHCEALRGSPLILY
RFFHEDVTLGNRSSPSGGASLNLSLTAEHSGNYSCEADNGLGAQRSETV
TLYITGLTANRSGPFATGVAGGLLSIAGLAAGALLLYCWLSRKAGRKPA
SDPARSPSDSDSQEPTYHNVPAWEELQPVYTNANPRGENVVYSEVRIIQ
EKKKHAVASDPRHLRNKGSPIIYSEVKVASTPVSGSLFLASSAPHR.

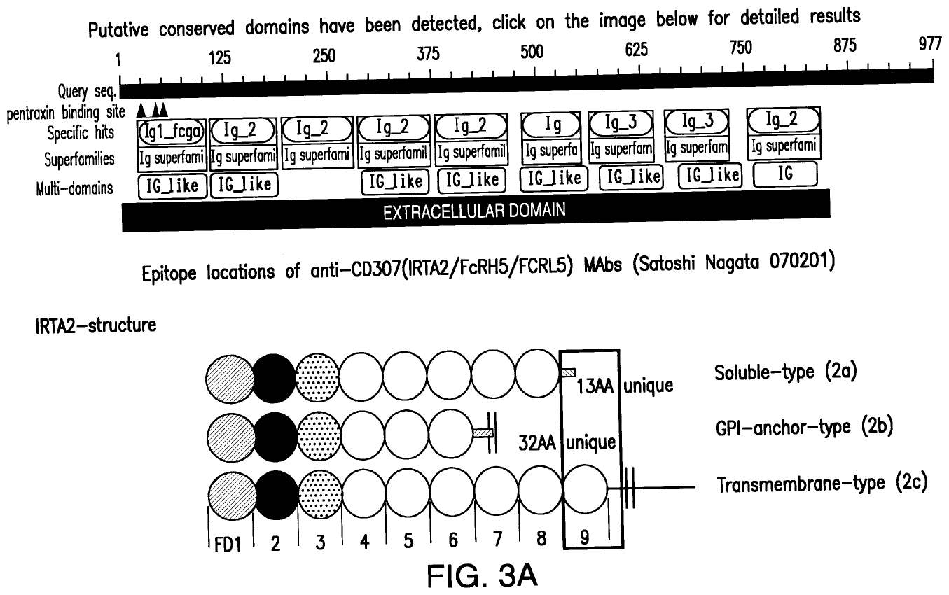

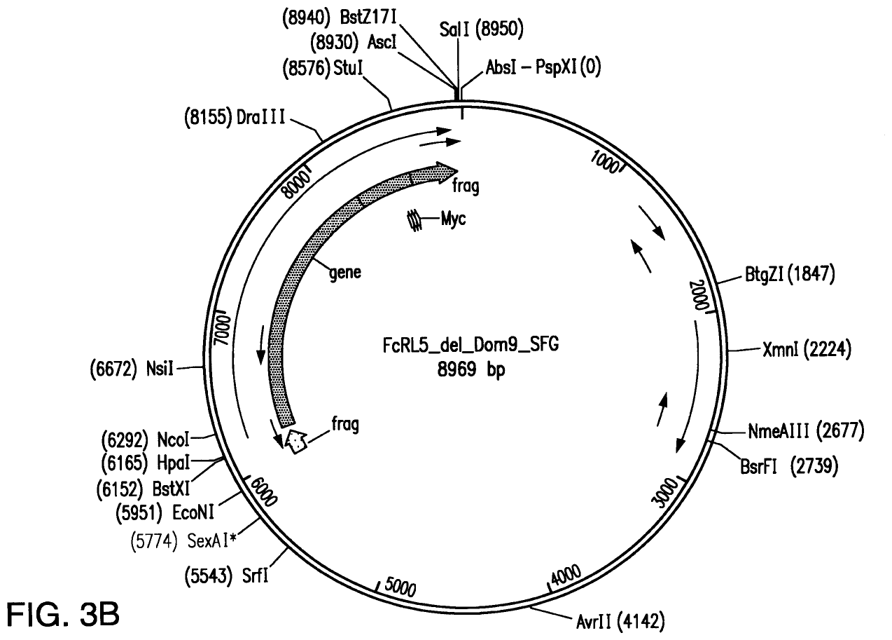

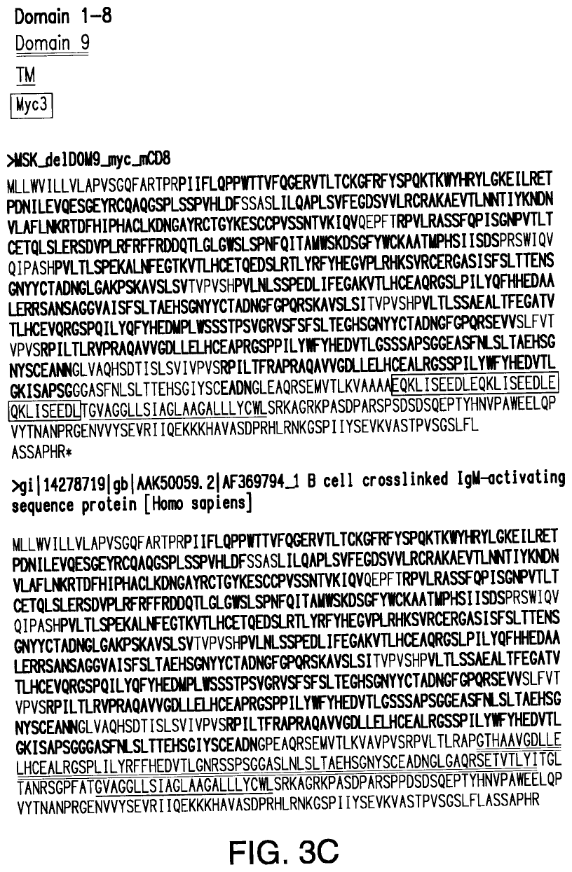

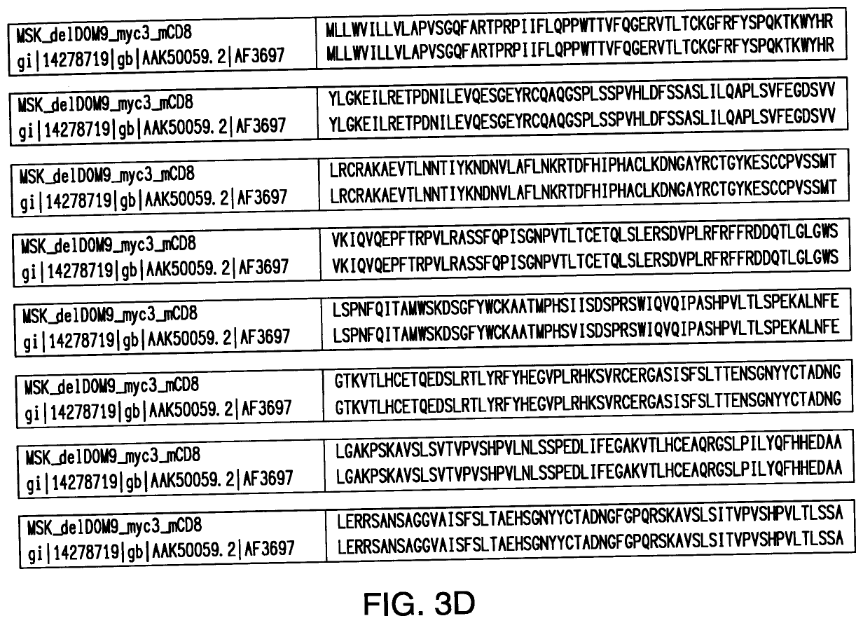

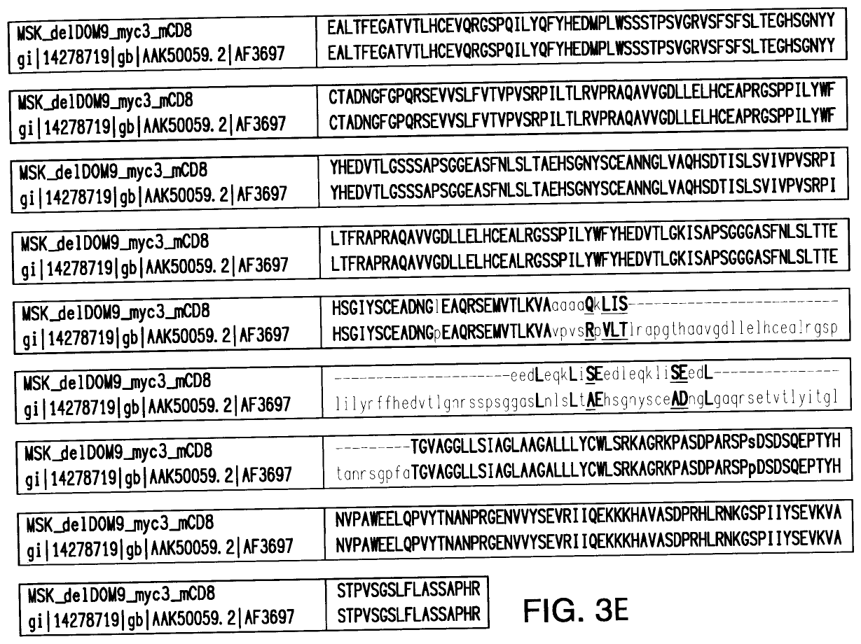

In certain embodiments, FcRL5 comprises 9 immunoglobulin (Ig)-like domains, e.g., domain 1, domain 2, domain 3, domain 4, domain 5, domain 6, domain 7, domain 8 and domain 9 (see FIGS. 3A and 3C). In certain embodiments, domain 9 of FcRL5 comprises the amino acid sequence set forth in SEQ ID NO:900. SEQ ID NO:900 is provided below.

[SEQ ID NO: 900]
RPVLTLRAPGTHAAVGDLLELHCEALRGSPLILYRFFHEDVTLGNRSSP
SGGASLNLSLTAEHSGNYSCEADNGLGAQRSETVTLYI.

In certain embodiments, domain 9 of FcRL5 can have the amino acid sequence set forth in SEQ ID NO:963, or fragments thereof. SEQ ID NO:963 is provided below:

[SEQ ID NO: 963]
GTHAAVGDLLELHCEALRGSPLILYRFFHEDVTLGNRSSPSGGASLNLSL
TAEHSGNYSCEADNGLGAQRSETVTLYI.

In certain embodiments, domain 1 can comprise amino acids 23-100 of SEQ ID NO:899; domain 2 can comprise amino acids 105-185 of SEQ ID NO:899; domain 3 can comprise amino acids 191-273 of SEQ ID NO:899; domain 4 can comprise amino acids 287-373 of SEQ ID NO:899; domain 5 can comprise amino acids 380-466 of SEQ ID NO:899; domain 6 can comprise amino acids 490-555 of SEQ ID NO:899; domain 7 can comprise amino acids 565-638 of SEQ ID NO:899; domain 8 can comprise amino acids 658-731 of SEQ ID NO:899; and domain 9 can comprise amino acids 754-835 of SEQ ID NO:899.

In certain embodiments, domain 9 of FcRL5 comprises an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence of SEQ ID NO:900 or 963.

### III. Chimeric Antigen Receptor (CAR).

Chimeric antigen receptors (CARs) are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen binding domain (e.g., a single-chain variable fragments (scFv)) fused to a transmembrane domain, fused to cytoplasmic/intracellular domain of the T cell receptor chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL). “Third generation” CARs comprise those that provide multiple co-stimulation (e.g., CD28 and 4-1BB) and activation (CD3ζ).

In accordance with the presently disclosed subject matter, the CARs comprise an extracellular antigen-binding domain, a transmembrane domain and an intracellular domain, where the extracellular antigen-binding domain binds to FcRL5. In a specific non-limiting embodiment, the extracellular antigen-binding domain is a scFv. In a specific non-limiting embodiment, the extracellular antigen-binding domain is a Fab, which is optionally crosslinked. In a specific non-limiting embodiment, the extracellular binding domain is a F(ab)₂. In a specific non-limiting embodiment, any of the foregoing molecules may be comprised in a fusion protein with a heterologous sequence to form the extracellular antigen-binding domain.

In certain non-limiting embodiments, the extracellular antigen-binding domain of a CAR of the present disclosure has a high binding specificity as well as high binding affinity to FcRL5 or domain 9 of FcRL5. In certain non-limiting embodiments, the extracellular antigen-binding domain of a CAR of the present disclosure has a high binding specificity as well as high binding affinity to domain 8 of FcRL5. In certain non-limiting embodiments, the extracellular antigen-binding domain of a CAR of the present disclosure has a high binding specificity as well as high binding affinity to domain 7 of FcRL5. For example, in such embodiments, the extracellular antigen-binding domain of the CAR (embodied, for example, in an scFv or an analog thereof) binds to FcRL5 (or domain 8 or domain 9 of FcRL5) with a dissociation constant (K_{d}) of about 3×10⁻⁶ M or less. In certain embodiments, the K_{d} is about 1×10⁻⁶ M or less, about 1×10⁻⁷ M or less, about 1×10⁻⁸ M or less, about 1×10⁻⁹ M or less, about 1×10⁻¹⁰ M or less or about 1×10⁻¹¹ M or less. In certain embodiments, the K_{d} is from about 1×10⁻¹¹ M to about 3×10⁻⁶ M, 1×10⁻¹⁰ M to about 3×10⁻⁶ M or from about 1×10⁻⁹ M to about 3×10⁻⁶ M, such as from about 1×10⁻⁹ M to about 1×10⁻⁸ M, from about 1×10⁻⁸ M to about 1×10⁻⁷ M, or from about 1×10⁻⁷ M to about 1×10⁻⁶ M, or from about 1×10⁻⁶ M to about 3×10⁻⁶ M.

Binding of the extracellular antigen-binding domain (embodiment, for example, in an scFv or an analog thereof) of a presently disclosed CAR to FcRL5 (or domain 8 or domain 9 of FcRL5) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the FcRL5-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the FcRL5-targeted human scFv is labeled with GFP.

In certain embodiments, the extracellular antigen-binding domain of a presently disclosed CAR comprises a single-chain variable fragment (scFv). In one specific embodiment, the extracellular antigen-binding domain of a presently disclosed CAR comprises a human scFv that specifically binds to human FcRL5. In another specific embodiment, the extracellular antigen-binding domain of a presently disclosed CAR comprises a murine scFv that specifically binds to human FcRL5. In certain embodiments, the extracellular antigen-binding domain of a presently disclosed CAR comprises a scFv that specifically binds to at least a portion of domain 7 of FcRL5. In certain embodiments, the extracellular antigen-binding domain of a presently disclosed CAR comprises a scFv that specifically binds to at least a portion of domain 8 of FcRL5. In certain embodiments, the extracellular antigen-binding domain of a presently disclosed CAR comprises a scFv that specifically binds to at least a portion of domain 9 of FcRL5. For example, and not by way of limitation, domain 9 of FcRL5 comprises the amino acid sequence set forth in SEQ ID NO:900 or 963, or fragments thereof.

In certain embodiments, the extracellular antigen-binding domain is a murine scFv obtained from two commercially available mouse hybridomas binding different extracellular epitopes on human FcRL5, which have been characterized in the Franco et al., Journal of Immunology (2013);190:5739-5746; Ise et al., Clinical cancer research: an official fournal of the American Association for Cancer Research (2005);11:87-96; and Ise et al., Clinical chemistry and laboratory medicine: CCLM/FESCC (2006);44:594-602, each of which are herein incorporated by reference in their entireties. In certain embodiments, the extracellular antigen-binding domain is a murine scFv that is derived from a heavy chain variable region and a light chain variable region of an antibody that binds to human FcRL5, e.g., antibodies F56 and F119 as disclosed in Ise et al. (2005), which is herein incorporated by reference in its entirety.

### Extracellular Antigen-Binding Domain of A CAR

In certain embodiments, the extracellular antigen-binding domain (e.g., scFv) comprises a light chain variable region comprising an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917 and SEQ ID NO:921, wherein the scFv antibody binds to an FcRL5 polypeptide.

In certain embodiments, the extracellular antigen-binding domain (e.g., scFv) comprises a heavy chain variable region comprising an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915 and SEQ ID NO:919.

In certain embodiments, the extracellular antigen-binding domain (e.g., scFv) comprises a heavy chain variable region comprising amino acids having the sequence set forth in SEQ ID NO:915 as provided below.

[SEQ ID NO: 915]
VKLQESGGGLVQPGGSRKLSCAASGFTFSIFGLHWVRQAPEKGLEWVAYI
SGDSNTIYYADTVKGRFTISRDNPKNTLFLQMTSLRSEDTAMYYCARNSY
YALDYWGQGTTVTVSS

The nucleic acid sequence encoding the amino acid sequence of SEQ ID NO:915 is set forth in SEQ ID NO:916 as provided below.

[SEQ ID NO: 916]
GTGAAGCTGCAGGAGTCTGGGGGAGGCTTAGTGCAGCCTGGAGGGTCCCG
GAAACTCTCCTGTGCAGCCTCTGGATTCACTTTCAGTATCTTTGGATTGC
ACTGGGTTCGTCAGGCTCCAGAGAAGGGGCTGGAGTGGGTCGCATACATT
AGTGGTGACAGTAATACCATCTACTATGCAGACACAGTGAAGGGCCGATT
CACCATCTCCAGAGACAATCCCAAGAACACCCTGTTCCTGCAAATGACCA
GTCTAAGGTCTGAGGACACGGCCATGTATTACTGTGCAAGAAATAGCTAC
TATGCTCTGGACTACTGGGGCCAAGGGACCACGGTCACCGTCTCCTCA

In certain embodiments, the extracellular antigen-binding domain (e.g., scFv) comprises a light chain variable region comprising amino acids having the sequence set forth in SEQ ID NO:917 as provided below.

[SEQ ID NO: 917]
DIELTQSPAIMSVSPGEKVTMTCRASSSVSSSYLHWYQQRSGASPKIWIY
STSNLASGVPARFSGSGTGTSYSLTISSVEAEDAATYYCQQYSGYPWTFG
GGTKLEI

The nucleic acid sequence encoding the amino acid sequence of SEQ ID NO:917 is set forth in SEQ ID NO:918 as provided below.

[SEQ ID NO: 918]
GACATTGAGCTCACCCAGTCTCCAGCAATCATGTCTGTATCTCCAGGTGA
AAAGGTCACCATGACCTGCAGGGCCAGCTCAAGTGTCAGTTCCAGTTACT
TGCACTGGTACCAGCAGAGGTCAGGTGCCTCCCCCAAAATCTGGATTTAT
AGCACATCCAACTTGGCTTCTGGAGTCCCTGCTCGCTTCAGTGGCAGTGG
GACTGGGACCTCTTACTCTCTCACAATCAGCAGTGTGGAGGCTGAAGATG
CTGCCACTTATTACTGCCAGCAGTACAGTGGTTACCCGTGGACGTTCGGT
GGAGGGACCAAGCTGGAGATC

In certain embodiments, the extracellular antigen-binding domain (e.g., scFv) comprises a heavy chain variable region comprising amino acids having the sequence set forth in SEQ ID NO:919 as provided below.

[SEQ ID NO: 919]
VQLQESGGGLVQPGGSRKLSCTASGFTFSSFGMHWVRQAPEKGLEWVAYI
SSGSNNIYFADTVKGRFTISRDNPKNTLFLQMTSLRSEDTAMYYCARSEY
YGSSHMDYWGQGTTVTVSS

The nucleic acid sequence encoding the amino acid sequence of SEQ ID NO:3 is set forth in SEQ ID NO:920 as provided below.

[SEQ ID NO: 920]
GTCCAACTGCAGGAGTCAGGGGGAGGCTTAGTGCAGCCTGGAGGGTCCCG
GAAACTCTCCTGTACAGCCTCTGGATTCACTTTCAGTAGCTTTGGAATGC
ACTGGGTTCGTCAGGCTCCAGAGAAGGGGCTGGAGTGGGTCGCATACATT
AGTAGTGGCAGTAATAACATCTACTTTGCGGACACAGTGAAGGGCCGATT
CACCATCTCCAGAGACAATCCCAAGAACACCCTGTTCCTGCAAATGACCA
GTCTAAGGTCTGAGGACACGGCCATGTATTACTGTGCAAGATCGGAATAC
TACGGTAGTAGCCATATGGACTACTGGGGCCAAGGGACCACGGTCACCGT
CTCCTCA

In certain embodiments, the extracellular antigen-binding domain (e.g., scFv) comprises a light chain variable region comprising amino acids having the sequence set forth in SEQ ID NO:921 as provided below.

[SEQ ID NO: 921]
DIELTQSPKFMSTSVGDRVSVTCKASQNVGTNVAWYQQKPGQSPKPLIYS
ATYRNSGVPDRFTGSGSGTDFTLTITNVQSKDLADYFCQQYNRYPYTSGG
GTKLEI

The nucleic acid sequence encoding the amino acid sequence of SEQ ID NO:921 is set forth in SEQ ID NO:922 as provided below.

[SEQ ID NO: 922]
GACATTGAGCTCACCCAGTCTCCAAAATTCATGTCCACATCAGTAGGAGA
CAGGGTCAGCGTCACCTGCAAGGCCAGTCAGAATGTGGGTACTAATGTAG
CCTGGTATCAACAGAAACCAGGACAATCTCCTAAACCACTGATTTACTCG
GCAACCTACCGGAACAGTGGAGTCCCTGATCGCTTCACAGGCAGTGGATC
TGGGACAGATTTCACTCTCACCATCACTAACGTGCAGTCTAAAGACTTGG
CAGACTATTTCTGTCAACAATATAACAGGTATCCGTACACGTCCGGAGGG
GGGACCAAGCTGGAGATC

In certain embodiments, the extracellular antigen-binding domain (e.g., scFv) comprises a heavy chain variable region comprising amino acids having the sequence set forth in SEQ ID NO:144. The nucleic acid sequence encoding the amino acid sequence of SEQ ID NO: 144 is set forth in SEQ ID NO:142.

In certain embodiments, the extracellular antigen-binding domain (e.g., scFv) comprises a light chain variable region comprising amino acids having the sequence set forth in SEQ ID NO:143. The nucleic acid sequence encoding the amino acid sequence of SEQ ID NO:143 is set forth in SEQ ID NO:141.

In certain embodiments, the extracellular antigen-binding domain (e.g., scFv) comprises a heavy chain variable region comprising amino acids having the sequence set forth in SEQ ID NO:216. The nucleic acid sequence encoding the amino acid sequence of SEQ ID NO:216 is set forth in SEQ ID NO:214.

In certain embodiments, the extracellular antigen-binding domain (e.g., scFv) comprises a light chain variable region comprising amino acids having the sequence set forth in SEQ ID NO:215. The nucleic acid sequence encoding the amino acid sequence of SEQ ID NO:215 is set forth in SEQ ID NO:213.

In certain embodiments, the extracellular antigen-binding domain (e.g., scFv) comprises a heavy chain variable region comprising amino acids having the sequence set forth in SEQ ID NO:220. The nucleic acid sequence encoding the amino acid sequence of SEQ ID NO:220 is set forth in SEQ ID NO:218.

In certain embodiments, the extracellular antigen-binding domain (e.g., scFv) comprises a light chain variable region comprising amino acids having the sequence set forth in SEQ ID NO:219. The nucleic acid sequence encoding the amino acid sequence of SEQ ID NO:219 is set forth in SEQ ID NO:217.

In certain embodiments, the extracellular antigen-binding domain (e.g., scFv) comprises a heavy chain variable region comprising amino acids having the sequence set forth in SEQ ID NO:236. The nucleic acid sequence encoding the amino acid sequence of SEQ ID NO:236 is set forth in SEQ ID NO:234.

In certain embodiments, the extracellular antigen-binding domain (e.g., scFv) comprises a light chain variable region comprising amino acids having the sequence set forth in SEQ ID NO:235. The nucleic acid sequence encoding the amino acid sequence of SEQ ID NO:235 is set forth in SEQ ID NO:232.

In certain embodiments, the extracellular antigen-binding domain (e.g., scFv) comprises a heavy chain variable region comprising amino acids having the sequence set forth in SEQ ID NO:268. The nucleic acid sequence encoding the amino acid sequence of SEQ ID NO:268 is set forth in SEQ ID NO:266.

In certain embodiments, the extracellular antigen-binding domain (e.g., scFv) comprises a light chain variable region comprising amino acids having the sequence set forth in SEQ ID NO:267. The nucleic acid sequence encoding the amino acid sequence of SEQ ID NO:267 is set forth in SEQ ID NO:265.

In certain embodiments, the extracellular antigen-binding domain (e.g., scFv) comprises a heavy chain variable region comprising amino acids having the sequence set forth in SEQ ID NO:172. The nucleic acid sequence encoding the amino acid sequence of SEQ ID NO: 172 is set forth in SEQ ID NO:170.

In certain embodiments, the extracellular antigen-binding domain (e.g., scFv) comprises a light chain variable region comprising amino acids having the sequence set forth in SEQ ID NO:171. The nucleic acid sequence encoding the amino acid sequence of SEQ ID NO: 171 is set forth in SEQ ID NO:169.

In certain embodiments, the extracellular antigen-binding domain (e.g., scFv) comprises a heavy chain variable region comprising amino acids having the sequence set forth in SEQ ID NO:116. The nucleic acid sequence encoding the amino acid sequence of SEQ ID NO:116 is set forth in SEQ ID NO:114.

In certain embodiments, the extracellular antigen-binding domain (e.g., scFv) comprises a light chain variable region comprising amino acids having the sequence set forth in SEQ ID NO:115. The nucleic acid sequence encoding the amino acid sequence of SEQ ID NO: 115 is set forth in SEQ ID NO:113.

In certain embodiments, the extracellular antigen-binding domain (e.g., scFv) comprises (a) a light chain variable region comprising an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917, SEQ ID NO:921 and (b) a heavy chain variable region comprising an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:917 and SEQ ID NO:921, wherein the extracellular binding domain binds to an FcRL5 polypeptide.

In certain embodiments, the extracellular antigen-binding domain (e.g., scFv) comprises (a) a light chain variable region comprising amino acids having a sequence set forth in SEQ ID NO:3, and (b) a heavy chain variable region comprising amino acids having a sequence set forth in SEQ ID NO:4.

In certain embodiments, the extracellular antigen-binding domain (e.g., scFv) comprises (a) a light chain variable region comprising amino acids having a sequence set forth in SEQ ID NO:7, and (b) a heavy chain variable region comprising amino acids having a sequence set forth in SEQ ID NO:8.

In certain embodiments, the extracellular antigen-binding domain (e.g., scFv) comprises (a) a light chain variable region comprising amino acids having a sequence set forth in SEQ ID NO:11, and (b) a heavy chain variable region comprising amino acids having a sequence set forth in SEQ ID NO:12.

In certain embodiments, the extracellular antigen-binding domain (e.g., scFv) comprises (a) a light chain variable region comprising amino acids having a sequence set forth in SEQ ID NO:15, and (b) a heavy chain variable region comprising amino acids having a sequence set forth in SEQ ID NO:16.

In certain embodiments, the extracellular antigen-binding domain (e.g., scFv) comprises (a) a light chain variable region comprising amino acids having a sequence set forth in SEQ ID NO:19, and (b) a heavy chain variable region comprising amino acids having a sequence set forth in SEQ ID NO:20.

In certain embodiments, the extracellular antigen-binding domain (e.g., scFv) comprises (a) a light chain variable region comprising amino acids having a sequence set forth in SEQ ID NO:23, and (b) a heavy chain variable region comprising amino acids having a sequence set forth in SEQ ID NO:24.

In certain embodiments, the extracellular antigen-binding domain (e.g., scFv) comprises (a) a light chain variable region comprising amino acids having a sequence set forth in SEQ ID NO:27, and (b) a heavy chain variable region comprising amino acids having a sequence set forth in SEQ ID NO:28.

In certain embodiments, the extracellular antigen-binding domain (e.g., scFv) comprises (a) a light chain variable region comprising amino acids having a sequence set forth in SEQ ID NO:31, and (b) a heavy chain variable region comprising amino acids having a sequence set forth in SEQ ID NO:32.

In certain embodiments, the extracellular antigen-binding domain (e.g., scFv) comprises (a) a light chain variable region comprising amino acids having a sequence set forth in SEQ ID NO:35, and (b) a heavy chain variable region comprising amino acids having a sequence set forth in SEQ ID NO:36.

In certain embodiments, the extracellular antigen-binding domain (e.g., scFv) comprises (a) a light chain variable region comprising amino acids having a sequence set forth in SEQ ID NO:39, and (b) a heavy chain variable region comprising amino acids having a sequence set forth in SEQ ID NO:40.

In certain embodiments, the extracellular antigen-binding domain (e.g., scFv) comprises (a) a light chain variable region comprising amino acids having a sequence set forth in SEQ ID NO:43, and (b) a heavy chain variable region comprising amino acids having a sequence set forth in SEQ ID NO:44.

In certain embodiments, the extracellular antigen-binding domain (e.g., scFv) comprises (a) a light chain variable region comprising amino acids having a sequence set forth in SEQ ID NO:47, and (b) a heavy chain variable region comprising amino acids having a sequence set forth in SEQ ID NO:48.

In certain embodiments, the extracellular antigen-binding domain (e.g., scFv) comprises (a) a light chain variable region comprising amino acids having a sequence set forth in SEQ ID NO:51, and (b) a heavy chain variable region comprising amino acids having a sequence set forth in SEQ ID NO:52.

In certain embodiments, the extracellular antigen-binding domain (e.g., scFv) comprises (a) a light chain variable region comprising amino acids having a sequence set forth in SEQ ID NO:55, and (b) a heavy chain variable region comprising amino acids having a sequence set forth in SEQ ID NO:56.

In certain embodiments, the extracellular antigen-binding domain (e.g., scFv) comprises (a) a light chain variable region comprising amino acids having a sequence set forth in SEQ ID NO:59, and (b) a heavy chain variable region comprising amino acids having a sequence set forth in SEQ ID NO:60.

In certain embodiments, the extracellular antigen-binding domain (e.g., scFv) comprises (a) a light chain variable region comprising amino acids having a sequence set forth in SEQ ID NO:63, and (b) a heavy chain variable region comprising amino acids having a sequence set forth in SEQ ID NO:64.

In certain embodiments, the extracellular antigen-binding domain (e.g., scFv) comprises (a) a light chain variable region comprising amino acids having a sequence set forth in SEQ ID NO:67, and (b) a heavy chain variable region comprising amino acids having a sequence set forth in SEQ ID NO:68.

In certain embodiments, the extracellular antigen-binding domain (e.g., scFv) comprises (a) a light chain variable region comprising amino acids having a sequence set forth in SEQ ID NO:71, and (b) a heavy chain variable region comprising amino acids having a sequence set forth in SEQ ID NO:72.

In certain embodiments, the extracellular antigen-binding domain (e.g., scFv) comprises (a) a light chain variable region comprising amino acids having a sequence set forth in SEQ ID NO:75, and (b) a heavy chain variable region comprising amino acids having a sequence set forth in SEQ ID NO:76.

In certain embodiments, the extracellular antigen-binding domain (e.g., scFv) comprises (a) a light chain variable region comprising amino acids having a sequence set forth in SEQ ID NO:79, and (b) a heavy chain variable region comprising amino acids having a sequence set forth in SEQ ID NO:80.

In certain embodiments, the extracellular antigen-binding domain (e.g., scFv) comprises (a) a light chain variable region comprising amino acids having a sequence set forth in SEQ ID NO:83, and (b) a heavy chain variable region comprising amino acids having a sequence set forth in SEQ ID NO:84.

In certain embodiments, the extracellular antigen-binding domain (e.g., scFv) comprises (a) a light chain variable region comprising amino acids having a sequence set forth in SEQ ID NO:87, and (b) a heavy chain variable region comprising amino acids having a sequence set forth in SEQ ID NO:88.

In certain embodiments, the extracellular antigen-binding domain (e.g., scFv) comprises (a) a light chain variable region comprising amino acids having a sequence set forth in SEQ ID NO:91, and (b) a heavy chain variable region comprising amino acids having a sequence set forth in SEQ ID NO:92.

In certain embodiments, the extracellular antigen-binding domain (e.g., scFv) comprises (a) a light chain variable region comprising amino acids having a sequence set forth in SEQ ID NO:95, and (b) a heavy chain variable region comprising amino acids having a sequence set forth in SEQ ID NO:96.

In certain embodiments, the extracellular antigen-binding domain (e.g., scFv) comprises (a) a light chain variable region comprising amino acids having a sequence set forth in SEQ ID NO:99, and (b) a heavy chain variable region comprising amino acids having a sequence set forth in SEQ ID NO:100.

In certain embodiments, the extracellular antigen-binding domain (e.g., scFv) comprises (a) a light chain variable region comprising amino acids having a sequence set forth in SEQ ID NO:103, and (b) a heavy chain variable region comprising amino acids having a sequence set forth in SEQ ID NO:104.

In certain embodiments, the extracellular antigen-binding domain (e.g., scFv) comprises (a) a light chain variable region comprising amino acids having a sequence set forth in SEQ ID NO:107, and (b) a heavy chain variable region comprising amino acids having a sequence set forth in SEQ ID NO:108.

In certain embodiments, the extracellular antigen-binding domain (e.g., scFv) comprises (a) a light chain variable region comprising amino acids having a sequence set forth in SEQ ID NO:111, and (b) a heavy chain variable region comprising amino acids having a sequence set forth in SEQ ID NO:112.

In certain embodiments, the extracellular antigen-binding domain (e.g., scFv) comprises (a) a light chain variable region comprising amino acids having a sequence set forth in SEQ ID NO:115, and (b) a heavy chain variable region comprising amino acids having a sequence set forth in SEQ ID NO:116.

In certain embodiments, the extracellular antigen-binding domain (e.g., scFv) comprises (a) a light chain variable region comprising amino acids having a sequence set forth in SEQ ID NO:119, and (b) a heavy chain variable region comprising amino acids having a sequence set forth in SEQ ID NO:120.

In certain embodiments, the extracellular antigen-binding domain (e.g., scFv) comprises (a) a light chain variable region comprising amino acids having a sequence set forth in SEQ ID NO:123, and (b) a heavy chain variable region comprising amino acids having a sequence set forth in SEQ ID NO:124.

In certain embodiments, the extracellular antigen-binding domain (e.g., scFv) comprises (a) a light chain variable region comprising amino acids having a sequence set forth in SEQ ID NO:127, and (b) a heavy chain variable region comprising amino acids having a sequence set forth in SEQ ID NO:128.

In certain embodiments, the extracellular antigen-binding domain (e.g., scFv) comprises (a) a light chain variable region comprising amino acids having a sequence set forth in SEQ ID NO:131, and (b) a heavy chain variable region comprising amino acids having a sequence set forth in SEQ ID NO:132.

In certain embodiments, the extracellular antigen-binding domain (e.g., scFv) comprises (a) a light chain variable region comprising amino acids having a sequence set forth in SEQ ID NO:135, and (b) a heavy chain variable region comprising amino acids having a sequence set forth in SEQ ID NO:136.

In certain embodiments, the extracellular antigen-binding domain (e.g., scFv) comprises (a) a light chain variable region comprising amino acids having a sequence set forth in SEQ ID NO:139, and (b) a heavy chain variable region comprising amino acids having a sequence set forth in SEQ ID NO:140.

In certain embodiments, the extracellular antigen-binding domain (e.g., scFv) comprises (a) a light chain variable region comprising amino acids having a sequence set forth in SEQ ID NO:143, and (b) a heavy chain variable region comprising amino acids having a sequence set forth in SEQ ID NO:144.

In certain embodiments, the extracellular antigen-binding domain (e.g., scFv) comprises (a) a light chain variable region comprising amino acids having a sequence set forth in SEQ ID NO:147, and (b) a heavy chain variable region comprising amino acids having a sequence set forth in SEQ ID NO:148.

In certain embodiments, the extracellular antigen-binding domain (e.g., scFv) comprises (a) a light chain variable region comprising amino acids having a sequence set forth in SEQ ID NO:151, and (b) a heavy chain variable region comprising amino acids having a sequence set forth in SEQ ID NO:152.

In certain embodiments, the extracellular antigen-binding domain (e.g., scFv) comprises (a) a light chain variable region comprising amino acids having a sequence set forth in SEQ ID NO:155, and (b) a heavy chain variable region comprising amino acids having a sequence set forth in SEQ ID NO:156.

In certain embodiments, the extracellular antigen-binding domain (e.g., scFv) comprises (a) a light chain variable region comprising amino acids having a sequence set forth in SEQ ID NO:159, and (b) a heavy chain variable region comprising amino acids having a sequence set forth in SEQ ID NO:160.

In certain embodiments, the extracellular antigen-binding domain (e.g., scFv) comprises (a) a light chain variable region comprising amino acids having a sequence set forth in SEQ ID NO:163, and (b) a heavy chain variable region comprising amino acids having a sequence set forth in SEQ ID NO:164.

In certain embodiments, the extracellular antigen-binding domain (e.g., scFv) comprises (a) a light chain variable region comprising amino acids having a sequence set forth in SEQ ID NO:167, and (b) a heavy chain variable region comprising amino acids having a sequence set forth in SEQ ID NO:168.

In certain embodiments, the extracellular antigen-binding domain (e.g., scFv) comprises (a) a light chain variable region comprising amino acids having a sequence set forth in SEQ ID NO:171, and (b) a heavy chain variable region comprising amino acids having a sequence set forth in SEQ ID NO:172.

In certain embodiments, the extracellular antigen-binding domain (e.g., scFv) comprises (a) a light chain variable region comprising amino acids having a sequence set forth in SEQ ID NO:175, and (b) a heavy chain variable region comprising amino acids having a sequence set forth in SEQ ID NO:176.

In certain embodiments, the extracellular antigen-binding domain (e.g., scFv) comprises (a) a light chain variable region comprising amino acids having a sequence set forth in SEQ ID NO:179, and (b) a heavy chain variable region comprising amino acids having a sequence set forth in SEQ ID NO:180.

In certain embodiments, the extracellular antigen-binding domain (e.g., scFv) comprises (a) a light chain variable region comprising amino acids having a sequence set forth in SEQ ID NO:183, and (b) a heavy chain variable region comprising amino acids having a sequence set forth in SEQ ID NO:184.

In certain embodiments, the extracellular antigen-binding domain (e.g., scFv) comprises (a) a light chain variable region comprising amino acids having a sequence set forth in SEQ ID NO:187, and (b) a heavy chain variable region comprising amino acids having a sequence set forth in SEQ ID NO:188.

In certain embodiments, the extracellular antigen-binding domain (e.g., scFv) comprises (a) a light chain variable region comprising amino acids having a sequence set forth in SEQ ID NO:191, and (b) a heavy chain variable region comprising amino acids having a sequence set forth in SEQ ID NO:192.

In certain embodiments, the extracellular antigen-binding domain (e.g., scFv) comprises (a) a light chain variable region comprising amino acids having a sequence set forth in SEQ ID NO:195, and (b) a heavy chain variable region comprising amino acids having a sequence set forth in SEQ ID NO:196.

In certain embodiments, the extracellular antigen-binding domain (e.g., scFv) comprises (a) a light chain variable region comprising amino acids having a sequence set forth in SEQ ID NO:199, and (b) a heavy chain variable region comprising amino acids having a sequence set forth in SEQ ID NO:200.

In certain embodiments, the extracellular antigen-binding domain (e.g., scFv) comprises (a) a light chain variable region comprising amino acids having a sequence set forth in SEQ ID NO:203, and (b) a heavy chain variable region comprising amino acids having a sequence set forth in SEQ ID NO:204.

In certain embodiments, the extracellular antigen-binding domain (e.g., scFv) comprises (a) a light chain variable region comprising amino acids having a sequence set forth in SEQ ID NO:207, and (b) a heavy chain variable region comprising amino acids having a sequence set forth in SEQ ID NO:208.

In certain embodiments, the extracellular antigen-binding domain (e.g., scFv) comprises (a) a light chain variable region comprising amino acids having a sequence set forth in SEQ ID NO:211, and (b) a heavy chain variable region comprising amino acids having a sequence set forth in SEQ ID NO:212.

In certain embodiments, the extracellular antigen-binding domain (e.g., scFv) comprises (a) a light chain variable region comprising amino acids having a sequence set forth in SEQ ID NO:215, and (b) a heavy chain variable region comprising amino acids having a sequence set forth in SEQ ID NO:216.

In certain embodiments, the extracellular antigen-binding domain (e.g., scFv) comprises (a) a light chain variable region comprising amino acids having a sequence set forth in SEQ ID NO:219, and (b) a heavy chain variable region comprising amino acids having a sequence set forth in SEQ ID NO:220.

In certain embodiments, the extracellular antigen-binding domain (e.g., scFv) comprises (a) a light chain variable region comprising amino acids having a sequence set forth in SEQ ID NO:223, and (b) a heavy chain variable region comprising amino acids having a sequence set forth in SEQ ID NO:224.

In certain embodiments, the extracellular antigen-binding domain (e.g., scFv) comprises (a) a light chain variable region comprising amino acids having a sequence set forth in SEQ ID NO:227, and (b) a heavy chain variable region comprising amino acids having a sequence set forth in SEQ ID NO:228.

In certain embodiments, the extracellular antigen-binding domain (e.g., scFv) comprises (a) a light chain variable region comprising amino acids having a sequence set forth in SEQ ID NO:231, and (b) a heavy chain variable region comprising amino acids having a sequence set forth in SEQ ID NO:232.

In certain embodiments, the extracellular antigen-binding domain (e.g., scFv) comprises (a) a light chain variable region comprising amino acids having a sequence set forth in SEQ ID NO:235, and (b) a heavy chain variable region comprising amino acids having a sequence set forth in SEQ ID NO:236.

In certain embodiments, the extracellular antigen-binding domain (e.g., scFv) comprises (a) a light chain variable region comprising amino acids having a sequence set forth in SEQ ID NO:239, and (b) a heavy chain variable region comprising amino acids having a sequence set forth in SEQ ID NO:240.

In certain embodiments, the extracellular antigen-binding domain (e.g., scFv) comprises (a) a light chain variable region comprising amino acids having a sequence set forth in SEQ ID NO:243, and (b) a heavy chain variable region comprising amino acids having a sequence set forth in SEQ ID NO:244.

In certain embodiments, the extracellular antigen-binding domain (e.g., scFv) comprises (a) a light chain variable region comprising amino acids having a sequence set forth in SEQ ID NO:247, and (b) a heavy chain variable region comprising amino acids having a sequence set forth in SEQ ID NO:248.

In certain embodiments, the extracellular antigen-binding domain (e.g., scFv) comprises (a) a light chain variable region comprising amino acids having a sequence set forth in SEQ ID NO:251, and (b) a heavy chain variable region comprising amino acids having a sequence set forth in SEQ ID NO:252.

In certain embodiments, the extracellular antigen-binding domain (e.g., scFv) comprises (a) a light chain variable region comprising amino acids having a sequence set forth in SEQ ID NO:255, and (b) a heavy chain variable region comprising amino acids having a sequence set forth in SEQ ID NO:256.

In certain embodiments, the extracellular antigen-binding domain (e.g., scFv) comprises (a) a light chain variable region comprising amino acids having a sequence set forth in SEQ ID NO:259, and (b) a heavy chain variable region comprising amino acids having a sequence set forth in SEQ ID NO:260.

In certain embodiments, the extracellular antigen-binding domain (e.g., scFv) comprises (a) a light chain variable region comprising amino acids having a sequence set forth in SEQ ID NO:263, and (b) a heavy chain variable region comprising amino acids having a sequence set forth in SEQ ID NO:264.

In certain embodiments, the extracellular antigen-binding domain (e.g., scFv) comprises (a) a light chain variable region comprising amino acids having a sequence set forth in SEQ ID NO:267, and (b) a heavy chain variable region comprising amino acids having a sequence set forth in SEQ ID NO:268.

In certain embodiments, the extracellular antigen-binding domain (e.g., scFv) comprises (a) a light chain variable region comprising amino acids having a sequence set forth in SEQ ID NO:271, and (b) a heavy chain variable region comprising amino acids having a sequence set forth in SEQ ID NO:272.

In certain embodiments, the extracellular antigen-binding domain (e.g., scFv) comprises (a) a light chain variable region comprising amino acids having a sequence set forth in SEQ ID NO:275, and (b) a heavy chain variable region comprising amino acids having a sequence set forth in SEQ ID NO:276.

In certain embodiments, the extracellular antigen-binding domain (e.g., scFv) comprises (a) a light chain variable region comprising amino acids having a sequence set forth in SEQ ID NO:279, and (b) a heavy chain variable region comprising amino acids having a sequence set forth in SEQ ID NO:280.

In certain embodiments, the extracellular antigen-binding domain (e.g., scFv) comprises (a) a light chain variable region comprising amino acids having a sequence set forth in SEQ ID NO:283, and (b) a heavy chain variable region comprising amino acids having a sequence set forth in SEQ ID NO:284.

In certain embodiments, the extracellular antigen-binding domain (e.g., scFv) comprises (a) a light chain variable region comprising amino acids having a sequence set forth in SEQ ID NO:287, and (b) a heavy chain variable region comprising amino acids having a sequence set forth in SEQ ID NO:288.

In certain embodiments, the extracellular antigen-binding domain (e.g., scFv) comprises (a) a light chain variable region comprising amino acids having a sequence set forth in SEQ ID NO:291, and (b) a heavy chain variable region comprising amino acids having a sequence set forth in SEQ ID NO:292.

In certain embodiments, the extracellular antigen-binding domain (e.g., scFv) comprises (a) a light chain variable region comprising amino acids having a sequence set forth in SEQ ID NO:279, and (b) a heavy chain variable region comprising amino acids having a sequence set forth in SEQ ID NO:280.

In certain embodiments, the extracellular antigen-binding domain (e.g., scFv) comprises (a) a light chain variable region comprising amino acids having a sequence set forth in SEQ ID NO:283, and (b) a heavy chain variable region comprising amino acids having a sequence set forth in SEQ ID NO:284.

In certain embodiments, the extracellular antigen-binding domain (e.g., scFv) comprises (a) a light chain variable region comprising amino acids having a sequence set forth in SEQ ID NO:287, and (b) a heavy chain variable region comprising amino acids having a sequence set forth in SEQ ID NO:288.

In certain embodiments, the extracellular antigen-binding domain (e.g., scFv) comprises (a) a light chain variable region comprising amino acids having a sequence set forth in SEQ ID NO:291, and (b) a heavy chain variable region comprising amino acids having a sequence set forth in SEQ ID NO:292.

In certain embodiments, the extracellular antigen-binding domain (e.g., scFv) comprises (a) a light chain variable region comprising amino acids having a sequence set forth in SEQ ID NO:295, and (b) a heavy chain variable region comprising amino acids having a sequence set forth in SEQ ID NO:296.

In certain embodiments, the extracellular antigen-binding domain (e.g., scFv) comprises (a) a light chain variable region comprising amino acids having a sequence set forth in SEQ ID NO:299, and (b) a heavy chain variable region comprising amino acids having a sequence set forth in SEQ ID NO:300.

In certain embodiments, the extracellular antigen-binding domain (e.g., scFv) comprises (a) a light chain variable region comprising amino acids having a sequence set forth in SEQ ID NO:303, and (b) a heavy chain variable region comprising amino acids having a sequence set forth in SEQ ID NO:304.

In certain embodiments, the extracellular antigen-binding domain (e.g., scFv) comprises (a) a heavy chain variable region comprising amino acids having the sequence set forth in SEQ ID NO:915, and (b) a light chain variable region comprising amino acids having the sequence set forth in SEQ ID NO:917.

In certain embodiments, the extracellular antigen-binding domain (e.g., scFv) comprises (a) a heavy chain variable region comprising amino acids having the sequence set forth in SEQ ID NO:919, and (b) a light chain variable region comprising amino acids having the sequence set forth in SEQ ID NO:921.

In certain embodiments, the extracellular antigen-binding domain (e.g., scFv) comprises heavy and light chain variable regions comprising amino acid sequences that are homologous to the amino acid sequences described herein and as disclosed in Tables 1-76. For example, and not by way of limitation, the extracellular antigen-binding domain (e.g., scFv) comprises a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917 and SEQ ID NO:921.

In certain embodiments, the extracellular antigen-binding domain (e.g., scFv) comprises a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915 and SEQ ID NO:919.

In certain embodiments, the extracellular antigen-binding domain (e.g., scFv) comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:115, SEQ ID NO:119, SEQ ID NO:123, SEQ ID NO:127, SEQ ID NO:131, SEQ ID NO:135, SEQ ID NO:139, SEQ ID NO:143, SEQ ID NO:147, SEQ ID NO:151, SEQ ID NO:155, SEQ ID NO:159, SEQ ID NO:163, SEQ ID NO:167, SEQ ID NO:171, SEQ ID NO:175, SEQ ID NO:179, SEQ ID NO:183, SEQ ID NO:187, SEQ ID NO:191, SEQ ID NO:195, SEQ ID NO:199, SEQ ID NO:203, SEQ ID NO:207, SEQ ID NO:211, SEQ ID NO:215, SEQ ID NO:219, SEQ ID NO:223, SEQ ID NO:227, SEQ ID NO:231, SEQ ID NO:235, SEQ ID NO:239, SEQ ID NO:243, SEQ ID NO:247, SEQ ID NO:251, SEQ ID NO:255, SEQ ID NO:259, SEQ ID NO:263, SEQ ID NO:267, SEQ ID NO:271, SEQ ID NO:275, SEQ ID NO:279, SEQ ID NO:283, SEQ ID NO:287, SEQ ID NO:291, SEQ ID NO:295, SEQ ID NO:299, SEQ ID NO:303, SEQ ID NO:917 and SEQ ID NO:921; and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:116, SEQ ID NO:120, SEQ ID NO:124, SEQ ID NO:128, SEQ ID NO:132, SEQ ID NO:136, SEQ ID NO:140, SEQ ID NO:144, SEQ ID NO:148, SEQ ID NO:152, SEQ ID NO:156, SEQ ID NO:160, SEQ ID NO:164, SEQ ID NO:168, SEQ ID NO:172, SEQ ID NO:176, SEQ ID NO:180, SEQ ID NO:184, SEQ ID NO:188, SEQ ID NO:192, SEQ ID NO:196, SEQ ID NO:200, SEQ ID NO:204, SEQ ID NO:208, SEQ ID NO:212, SEQ ID NO:216, SEQ ID NO:220, SEQ ID NO:224, SEQ ID NO:228, SEQ ID NO:232, SEQ ID NO:236, SEQ ID NO:240, SEQ ID NO:244, SEQ ID NO:248, SEQ ID NO:252, SEQ ID NO:256, SEQ ID NO:260, SEQ ID NO:264, SEQ ID NO:268, SEQ ID NO:272, SEQ ID NO:276, SEQ ID NO:280, SEQ ID NO:284, SEQ ID NO:288, SEQ ID NO:292, SEQ ID NO:296, SEQ ID NO:300, SEQ ID NO:304, SEQ ID NO:915 and SEQ ID NO:919.

In certain embodiments, the extracellular antigen-binding domain (e.g., scFv) comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:143, and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:144, wherein the extracellular antigen-binding domain binds to an FcRL5 polypeptide.

In certain embodiments, the extracellular antigen-binding domain (e.g., scFv) comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:215, and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:216, wherein the extracellular antigen-binding domain binds to an FcRL5 polypeptide.

In certain embodiments, the extracellular antigen-binding domain (e.g., scFv) comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:219, and (b) a heavy chain variable comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:220, wherein the extracellular antigen-binding domain binds to an FcRL5 polypeptide.

In certain embodiments, the extracellular antigen-binding domain (e.g., scFv) comprises (a) a light chain variable comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:235, and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:236, wherein the extracellular antigen-binding domain binds to an FcRL5 polypeptide.

In certain embodiments, the extracellular antigen-binding domain (e.g., scFv) comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:267, and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:268, wherein the extracellular antigen-binding domain binds to an FcRL5 polypeptide.

In certain embodiments, the extracellular antigen-binding domain (e.g., scFv) comprises (a) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:915, and (b) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:917, wherein the extracellular antigen-binding domain binds to an FcRL5 polypeptide.

In certain embodiments, the extracellular antigen-binding domain (e.g., scFv) comprises (a) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:919, and (b) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:921, wherein the extracellular antigen-binding domain binds to an FcRL5 polypeptide.

In certain embodiments, the extracellular antigen-binding domain (e.g., scFv) comprises (a) a light chain variable comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:115, and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:116, wherein the extracellular antigen-binding domain binds to an FcRL5 polypeptide.

In certain embodiments, the extracellular antigen-binding domain (e.g., scFv) comprises (a) a light chain variable comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:171, and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:172, wherein the extracellular antigen-binding domain binds to an FcRL5 polypeptide.

An extracellular antigen-binding domain (e.g., scFv) comprising V_{H} and/or V_{L} regions having high (i.e., 80% or greater) homology to the V_{H} and V_{L} regions of the sequences set forth above, can be obtained by mutagenesis (e.g., site-directed or PCR-mediated mutagenesis), followed by testing of the encoded altered scFv for retained function (i.e., the binding affinity) using the binding assays described herein. In certain embodiments, a V_{L} sequence having at least 90%, 91%, 92%, 93%, 94%, 95%, 96%, 97%, 98% or 99% identity contains substitutions (e.g., conservative substitutions to generate conservative modifications of a sequence), insertions or deletions relative to the reference sequence, but an extracellular antigen-binding domain (e.g., scFv) comprising that sequence retains the ability to bind to FcRL5. In certain embodiments, a V_{H} sequence having at least 90%, 91%, 92%, 93%, 94%, 95%, 96%, 97%, 98%, or 99% identity contains substitutions (e.g., conservative substitutions), insertions or deletions relative to the reference sequence, but an extracellular antigen-binding domain (e.g., scFv) comprising that sequence retains the ability to bind to FcRL5. In certain embodiments, a total of about 1 to about 10 amino acids have been substituted, inserted and/or deleted in the disclosed sequences. For example, and not by way of limitation, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted. Non-limiting examples of conservative modifications are provided below, e.g., within Table 231.

The presently disclosed subject matter further provides extracellular antigen-binding domains (e.g., scFv) that comprise heavy chain variable region and light chain variable region CDRs, e.g., CDR1s, CDR2s and CDR3s, as disclosed herein in Tables 229 and 230. The CDR regions are delineated using the Kabat system (Kabat, E. A., et al. (1991) Sequences of Proteins of Immunological Interest, Fifth Edition, U.S. Department of Health and Human Services, NIH Publication No. 91-3242). The present disclosure further provides extracellular antigen-binding domains (e.g., scFv) that comprise conservative modifications of the antibody sequences disclosed herein. For example, and not by way of limitation, an extracellular antigen-binding domains (e.g., scFv) of the presently disclosed subject matter comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences disclosed herein, or conservative modifications thereof, and wherein the extracellular antigen-binding domains retain the desired functional properties. See Tables 229 and 230.

In certain embodiments, the presently disclosed subject matter provides an extracellular antigen-binding domain (e.g., scFv) comprising a light chain variable region, wherein the light chain variable region comprises: (a) a CDR1 comprising an amino acid sequence selected from the group consisting of SEQ ID NOs: 312, 3118, 324, 329, 338, 343, 348, 352, 357, 363, 369, 381, 390, 397, 401, 406, 416, 423, 428, 433, 447, 460, 468, 474, 477, 483, 490, 498, 503, 508, 518, 533, 540, 544, 547, 556, 562, 568, 571, 580, 585, 588, 926 and 932, and conservative modifications thereof; (b) a CDR2 comprising an amino acid sequence selected from the group consisting of SEQ ID NOs:313, 319, 330, 344, 349, 358, 364, 370, 382, 385, 391, 398, 409, 417, 429, 434, 438, 448, 454, 461, 469, 478, 484, 487, 504, 513, 523, 534, 429, 448, 548, 557, 563, 572, 575, 586, 927 and 933, and conservative modifications thereof; and (c) a CDR3 comprising an amino acid sequence selected from the group consisting of SEQ ID NOs: 314, 320, 325, 331, 339, 345, 350, 353, 359, 365, 371, 377, 383, 386, 392, 395, 399, 402, 407, 410, 414, 418, 419, 424, 430, 435, 439, 443, 449, 452, 455, 457, 462, 465, 470, 479, 485, 488, 491, 493, 495, 499, 505, 509, 514, 519, 524, 528, 530, 531, 535, 541, 542, 545, 549, 554, 558, 564, 569, 573, 576, 581, 592, 928 and 934, and conservative modifications thereof. In certain embodiments, the extracellular antigen-binding domain (e.g., scFv) comprises a heavy chain variable region, wherein the heavy chain variable region comprises: (a) a CDR1 comprising an amino acid sequence selected from the group consisting of SEQ ID NOs: 309, 315, 321, 326, 332, 335, 340, 346, 354, 360, 366, 372, 378, 387, 393, 403, 411, 420, 425, 436, 440, 444, 471, 480, 500, 510, 515, 520, 525, 537, 551, 559, 565, 582, 589, 923 and 929 and conservative modifications thereof; (b) a CDR2 comprising an amino acid sequence selected from the group consisting of SEQ ID NOs: 310, 316, 322, 327, 333, 336, 341, 355, 361, 367, 373, 379, 388, 404, 412, 421, 426, 431, 441, 445, 450, 466, 472, 475, 481, 496, 501, 506, 511, 516, 521, 526, 538, 552, 560, 566, 583, 590, 924 and 930 and conservative modifications thereof; and (c) a CDR3 comprising an amino acid sequence selected from the group consisting of SEQ ID NOs: 311, 317, 323, 328, 334, 337, 342, 347, 351, 356, 362, 368, 374, 376, 380, 384, 389, 394, 396, 400, 405, 408, 412, 415, 422, 427, 432, 437, 442, 446, 451, 453, 456, 458, 459, 463, 464, 467, 473, 476, 482, 486, 489, 492, 494, 497, 502, 507, 512, 517, 522, 527, 529, 532, 536, 539, 543, 546, 550, 553, 555, 561, 567, 570, 574, 577, 578, 579, 584, 578, 587, 591, 925 and 931, and conservative modifications thereof.

In certain embodiments, the extracellular antigen-binding domain (e.g., scFv) comprises (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; and (c) a heavy chain variable region CDR3 comprising the amino acid sequence of SEQ ID NO:463 or conservative modifications thereof. In certain embodiments, the extracellular antigen-binding domain (e.g., scFv) comprises (a) a light chain variable region CDR1 comprising the amino acid sequence of SEQ ID NO:318 or conservative modifications thereof; (b) a light chain variable region CDR2 comprising the amino acid sequence of SEQ ID NO:319 or conservative modifications thereof; and (c) a light chain variable region CDR3 comprising the amino acid sequence of SEQ ID NO:419 or conservative modifications thereof.

In certain embodiments, the extracellular antigen-binding domain (e.g., scFv) comprises (a) a heavy chain variable region CDR1 comprising the amino acid sequence of SEQ ID NO:515 or conservative modifications thereof; (b) a heavy chain variable region CDR2 comprising the amino acid sequence of SEQ ID NO:516 or conservative modifications thereof; and (c) a heavy chain variable region CDR3 comprising the amino acid sequence of SEQ ID NO:517 or conservative modifications thereof. In certain embodiments, the extracellular antigen-binding domain (e.g., scFv) comprises (a) a light chain variable region CDR1 comprising the amino acid sequence of SEQ ID NO:318 or conservative modifications thereof; (b) a light chain variable region CDR2 comprising the amino acid sequence of SEQ ID NO:319 or conservative modifications thereof; and (c) a light chain variable region CDR3 comprising the amino acid sequence of SEQ ID NO:531 or conservative modifications thereof.

In certain embodiments, the extracellular antigen-binding domain (e.g., scFv) comprises (a) a heavy chain variable region CDR1 comprising the amino acid sequence of SEQ ID NO:403 or conservative modifications thereof; (b) a heavy chain variable region CDR2 comprising the amino acid sequence of SEQ ID NO:404 or conservative modifications thereof; and (c) a heavy chain variable region CDR3 comprising the amino acid sequence of SEQ ID NO:532 or conservative modifications thereof. In certain embodiments, the extracellular antigen-binding domain (e.g., scFv) comprises (a) a light chain variable region CDR1 comprising the amino acid sequence of SEQ ID NO:533 or conservative modifications thereof; (b) a light chain variable region CDR2 comprising the amino acid sequence of SEQ ID NO:534 or conservative modifications thereof; and (c) a light chain variable region CDR3 comprising the amino acid sequence of SEQ ID NO:535 or conservative modifications thereof.

In certain embodiments, the extracellular antigen-binding domain (e.g., scFv) comprises (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; and (c) a heavy chain variable region CDR3 comprising the amino acid sequence of SEQ ID NO:543 or conservative modifications thereof. In certain embodiments, the extracellular antigen-binding domain (e.g., scFv) comprises (a) a light chain variable region CDR1 comprising the amino acid sequence of SEQ ID NO:544 or conservative modifications thereof; (b) a light chain variable region CDR2 comprising the amino acid sequence of SEQ ID NO:448 or conservative modifications thereof; and (c) a light chain variable region CDR3 comprising the amino acid sequence of SEQ ID NO:545 or conservative modifications thereof.

In certain embodiments, the extracellular antigen-binding domain (e.g., scFv) comprises (a) a heavy chain variable region CDR1 comprising the amino acid sequence of SEQ ID NO:372 or conservative modifications thereof; (b) a heavy chain variable region CDR2 comprising the amino acid sequence of SEQ ID NO:475 or conservative modifications thereof; and (c) a heavy chain variable region CDR3 comprising the amino acid sequence of SEQ ID NO:570 or conservative modifications thereof. In certain embodiments, the extracellular antigen-binding domain (e.g., scFv) comprises (a) a light chain variable region CDR1 comprising the amino acid sequence of SEQ ID NO:571 or conservative modifications thereof; (b) a light chain variable region CDR2 comprising the amino acid sequence of SEQ ID NO:572 or conservative modifications thereof; and (c) a light chain variable region CDR3 comprising the amino acid sequence of SEQ ID NO:573 or conservative modifications thereof.

In certain embodiments, the extracellular antigen-binding domain (e.g., scFv) comprises (a) a heavy chain variable region CDR1 comprising the amino acid sequence of SEQ ID NO:440 or conservative modifications thereof; (b) a heavy chain variable region CDR2 comprising the amino acid sequence of SEQ ID NO:441 or conservative modifications thereof; and (c) a heavy chain variable region CDR3 comprising the amino acid sequence of SEQ ID NO:442 or conservative modifications thereof. In certain embodiments, the extracellular antigen-binding domain (e.g., scFv) comprises (a) a light chain variable region CDR1 comprising the amino acid sequence of SEQ ID NO:329 or conservative modifications thereof; (b) a light chain variable region CDR2 comprising the amino acid sequence of SEQ ID NO:330 or conservative modifications thereof; and (c) a light chain variable region CDR3 comprising the amino acid sequence of SEQ ID NO:443 or conservative modifications thereof.

In certain embodiments, the extracellular antigen-binding domain (e.g., scFv) comprises (a) a heavy chain variable region CDR1 comprising the amino acid sequence of SEQ ID NO:309 or conservative modifications thereof; (b) a heavy chain variable region CDR2 comprising the amino acid sequence of SEQ ID NO:310 or conservative modifications thereof; and (c) a heavy chain variable region CDR3 comprising the amino acid sequence of SEQ ID NO:489 or conservative modifications thereof. In certain embodiments, the extracellular antigen-binding domain (e.g., scFv) comprises (a) a light chain variable region CDR1 comprising the amino acid sequence of SEQ ID NO:490 or conservative modifications thereof; (b) a light chain variable region CDR2 comprising the amino acid sequence of SEQ ID NO:313 or conservative modifications thereof; and (c) a light chain variable region CDR3 comprising the amino acid sequence of SEQ ID NO:491 or conservative modifications thereof.

In certain embodiments, the extracellular antigen-binding domain (e.g., scFv) comprises (a) a heavy chain variable region CDR1 comprising amino acids having the sequence set forth in SEQ ID NO:923 or conservative modifications thereof; (b) a heavy chain variable region CDR2 comprising amino acids having the sequence set forth in SEQ ID NO:924 or conservative modifications thereof; and (c) a heavy chain variable region CDR3 comprising amino acids having the sequence set forth in SEQ ID NO:925 or conservative modifications thereof. In certain embodiments, the extracellular antigen-binding domain comprises (a) a light chain variable region CDR1 comprising amino acids having the sequence set forth in SEQ ID NO:926 or conservative modifications thereof; (b) a light chain variable region CDR2 comprising amino acids having the sequence set forth in SEQ ID NO:927 or conservative modifications thereof; and (c) a light chain variable region CDR3 comprising amino acids having the sequence set forth in SEQ ID NO:928 or conservative modifications thereof.

In certain embodiments, the extracellular antigen-binding domain (e.g., scFv) comprises (a) a heavy chain variable region CDR1 comprising amino acids having the sequence set forth in SEQ ID NO:929 or conservative modifications thereof; (b) a heavy chain variable region CDR2 comprising amino acids having the sequence set forth in SEQ ID NO:930 or conservative modifications thereof; and (c) a heavy chain variable region CDR3 comprising amino acids having the sequence set forth in SEQ ID NO:931 or conservative modifications thereof. In certain embodiments, the extracellular antigen-binding domain comprises (a) a light chain variable region CDR1 comprising amino acids having the sequence set forth in SEQ ID NO:932 or conservative modifications thereof; (b) a light chain variable region CDR2 comprising amino acids having the sequence set forth in SEQ ID NO: 933 or conservative modifications thereof; and (c) a light chain variable region CDR3 comprising amino acids having the sequence set forth in SEQ ID NO:934 or conservative modifications thereof. The presently disclosed subject matter provides an extracellular antigen-binding domain (e.g., scFv) comprising a heavy chain variable region comprising CDR1, CDR2, and CDR3 sequences and a light chain variable region comprising CDR1, CDR2, and CDR3 sequences, wherein: (a) the heavy chain variable region CDR3 comprises an amino acid sequence selected from the group consisting of SEQ ID NOs: 311, 317, 323, 328, 334, 337, 342, 347, 351, 356, 362, 368, 374, 376, 380, 384, 389, 394, 396, 400, 405, 408, 412, 415, 422, 427, 432, 437, 442, 446, 451, 453, 456, 458, 459, 463, 464, 467, 473, 476, 482, 486, 489, 492, 494, 497, 502, 507, 512, 517, 522, 527, 529, 532, 536, 539, 543, 546, 550, 553, 555, 561, 567, 570, 574, 577, 578, 579, 584, 578, 587, 591, 925 and 931, and conservative modifications thereof; and (b) the light chain variable region CDR3 comprises an amino acid sequence selected from the group consisting of SEQ ID NOs: 314, 320, 325, 331, 339, 345, 350, 353, 359, 365, 371, 377, 383, 386, 392, 395, 399, 402, 407, 410, 414, 418, 419, 424, 430, 435, 439, 443, 449, 452, 455, 457, 462, 465, 470, 479, 485, 488, 491, 493, 495, 499, 505, 509, 514, 519, 524, 528, 530, 531, 535, 541, 542, 545, 549, 554, 558, 564, 569, 573, 576, 581, 592, 928 and 934 and conservative modifications thereof; wherein the extracellular antigen-binding domain specifically binds to human FcRL5.

In certain embodiments, the extracellular antigen-binding domain (e.g., scFv) comprises (a) a heavy chain variable region CDR3 comprising the amino acid sequence of SEQ ID NO:463 or conservative modifications thereof; and (b) a light chain variable region CDR3 comprising the amino acid sequence of SEQ ID NO:419 or conservative modifications thereof; wherein the extracellular antigen-binding domain specifically binds FcRL5.

In certain embodiments, the extracellular antigen-binding domain (e.g., scFv) comprises (a) a heavy chain variable region CDR3 comprising the amino acid sequence of SEQ ID NO:517 or conservative modifications thereof; and (b) a light chain variable region CDR3 comprising the amino acid sequence of SEQ ID NO:531 or conservative modifications thereof; wherein the extracellular antigen-binding domain specifically binds FcRL5.

In certain embodiments, the extracellular antigen-binding domain (e.g., scFv) comprises (a) a heavy chain variable region CDR3 comprising the amino acid sequence of SEQ ID NO:532 or conservative modifications thereof; and (b) a light chain variable region CDR3 comprising the amino acid sequence of SEQ ID NO:535 or conservative modifications thereof; wherein the antibody or antigen-binding fragment thereof specifically binds FcRL5.

In certain embodiments, the extracellular antigen-binding domain (e.g., scFv) comprises (a) a heavy chain variable region CDR3 comprising the amino acid sequence of SEQ ID NO:543 or conservative modifications thereof; and (b) a light chain variable region CDR3 comprising the amino acid sequence of SEQ ID NO:545 or conservative modifications thereof; wherein the extracellular antigen-binding domain specifically binds FcRL5.

In certain embodiments, the extracellular antigen-binding domain (e.g., scFv) comprises (a) a heavy chain variable region CDR3 comprising the amino acid sequence of SEQ ID NO:570 or conservative modifications thereof; and (b) a light chain variable region CDR3 comprising the amino acid sequence of SEQ ID NO:573 or conservative modifications thereof; wherein the antibody or antigen-binding fragment thereof specifically binds FcRL5.

In certain embodiments, the extracellular antigen-binding domain (e.g., scFv) comprises (a) a heavy chain variable region CDR3 comprising amino acids having the sequence set forth in SEQ ID NO:925 or conservative modifications thereof; and (b) and a light chain variable region CDR3 comprising amino acids having the sequence set forth in SEQ ID NO:928 or conservative modifications thereof; wherein the extracellular antigen-binding domain specifically binds FcRL5. In certain embodiments, the extracellular antigen-binding domain (e.g., scFv) comprises (a) a heavy chain variable region CDR3 comprising amino acids having the sequence set forth in SEQ ID NO:931 or conservative modifications thereof; and (b) a light chain variable region CDR3 comprising amino acids having the sequence set forth in SEQ ID NO:934 or conservative modifications thereof. In certain embodiments, the extracellular antigen-binding domain (e.g., scFv) comprises (a) a heavy chain variable region CDR3 comprising the amino acid sequence of SEQ ID NO:442 or conservative modifications thereof; and (b) a light chain variable region CDR3 comprising the amino acid sequence of SEQ ID NO:443 or conservative modifications thereof; wherein the extracellular antigen-binding domain specifically binds FcRL5.

In certain embodiments, the extracellular antigen-binding domain (e.g., scFv) comprises (a) a heavy chain variable region CDR3 comprising the amino acid sequence of SEQ ID NO:489 or conservative modifications thereof; and (b) a light chain variable region CDR3 comprising the amino acid sequence of SEQ ID NO:491 or conservative modifications thereof; wherein the extracellular antigen-binding domain specifically binds FcRL5.

In certain embodiments, the presently disclosed subject matter provides an extracellular antigen-binding domain (e.g., scFv) comprising a heavy chain variable region comprising CDR1, CDR2, and CDR3 sequences and a light chain variable region comprising CDR1, CDR2, and CDR3 sequences, wherein: (a) the heavy chain variable region CDR1 comprises an amino acid sequence selected from the group consisting of SEQ ID NOs: 309, 315, 321, 326, 332, 335, 340, 346, 354, 360, 366, 372, 378, 387, 393, 403, 411, 420, 425, 436, 440, 444, 471, 480, 500, 510, 515, 520, 525, 537, 551, 559, 565, 582, 589, 923 and 929, and conservative modifications thereof; (b) the heavy chain variable region CDR2 comprises an amino acid sequence selected from the group consisting of SEQ ID NOs: 310, 316, 322, 327, 333, 336, 341, 355, 361, 367, 373, 379, 388, 404, 412, 421, 426, 431, 441, 445, 450, 466, 472, 475, 481, 496, 501, 506, 511, 516, 521, 526, 538, 552, 560, 566, 583, 590, 924 and 930, and conservative modifications thereof; (c) the heavy chain variable region CDR3 comprises an amino acid sequence selected from the group consisting of SEQ ID NOs: 311, 317, 323, 328, 334, 337, 342, 347, 351, 356, 362, 368, 374, 376, 380, 384, 389, 394, 396, 400, 405, 408, 412, 415, 422, 427, 432, 437, 442, 446, 451, 453, 456, 458, 459, 463, 464, 467, 473, 476, 482, 486, 489, 492, 494, 497, 502, 507, 512, 517, 522, 527, 529, 532, 536, 539, 543, 546, 550, 553, 555, 561, 567, 570, 574, 577, 578, 579, 584, 578, 587, 591, 925 and 931 and conservative modifications thereof; (d) the light chain variable region CDR1 comprises an amino acid sequence selected from the group consisting of SEQ ID NOs: 312, 3118, 324, 329, 338, 343, 348, 352, 357, 363, 369, 381, 390, 397, 401, 406, 416, 423, 428, 433, 447, 460, 468, 474, 477, 483, 490, 498, 503, 508, 518, 533, 540, 544, 547, 556, 562, 568, 571, 580, 585, 588, 926 and 932 and conservative modifications thereof; (e) the light chain variable region CDR2 comprises an amino acid sequence selected from the group consisting of SEQ ID NOs:313, 319, 330, 344, 349, 358, 364, 370, 382, 385, 391, 398, 409, 417, 429, 434, 438, 448, 454, 461, 469, 478, 484, 487, 504, 513, 523, 534, 429, 448, 548, 557, 563, 572, 575, 586, 927 and 933 and conservative modifications thereof; and (f) the light chain variable region CDR3 comprises an amino acid sequence selected from the group consisting of SEQ ID NOs: 314, 320, 325, 331, 339, 345, 350, 353, 359, 365, 371, 377, 383, 386, 392, 395, 399, 402, 407, 410, 414, 418, 419, 424, 430, 435, 439, 443, 449, 452, 455, 457, 462, 465, 470, 479, 485, 488, 491, 493, 495, 499, 505, 509, 514, 519, 524, 528, 530, 531, 535, 541, 542, 545, 549, 554, 558, 564, 569, 573, 576, 581, 592, 928 and 934 and conservative modifications thereof; wherein the extracellular antigen-binding domain specifically binds FcRL5.

In certain embodiments, the extracellular antigen-binding domain (e.g., scFv) comprises (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; (c) a heavy chain variable region CDR3 comprising the amino acid sequence of SEQ ID NO:463 or conservative modifications thereof; (d) a light chain variable region CDR1 comprising the amino acid sequence of SEQ ID NO:318 or conservative modifications thereof; (e) a light chain variable region CDR2 comprising the amino acid sequence of SEQ ID NO:319 or conservative modifications thereof; and (f) a light chain variable region CDR3 comprising the amino acid sequence of SEQ ID NO:419 or conservative modifications thereof.

In certain embodiments, the extracellular antigen-binding domain (e.g., scFv) comprises (a) a heavy chain variable region CDR1 comprising the amino acid sequence of SEQ ID NO:515 or conservative modifications thereof; (b) a heavy chain variable region CDR2 comprising the amino acid sequence of SEQ ID NO:516 or conservative modifications thereof; (c) a heavy chain variable region CDR3 comprising the amino acid sequence of SEQ ID NO:517 or conservative modifications thereof; (d) a light chain variable region CDR1 comprising the amino acid sequence of SEQ ID NO:318 or conservative modifications thereof; (e) a light chain variable region CDR2 comprising the amino acid sequence of SEQ ID NO:319 or conservative modifications thereof; and (f) a light chain variable region CDR3 comprising the amino acid sequence of SEQ ID NO:531 or conservative modifications thereof.

In certain embodiments, the extracellular antigen-binding domain (e.g., scFv) comprises (a) a heavy chain variable region CDR1 comprising the amino acid sequence of SEQ ID NO:403 or conservative modifications thereof; (b) a heavy chain variable region CDR2 comprising the amino acid sequence of SEQ ID NO:404 or conservative modifications thereof; (c) a heavy chain variable region CDR3 comprising the amino acid sequence of SEQ ID NO:532 or conservative modifications thereof; (d) a light chain variable region CDR1 comprising the amino acid sequence of SEQ ID NO:533 or conservative modifications thereof; (e) a light chain variable region CDR2 comprising the amino acid sequence of SEQ ID NO:534 or conservative modifications thereof; and (f) a light chain variable region CDR3 comprising the amino acid sequence of SEQ ID NO:535 or conservative modifications thereof.

In certain embodiments, the extracellular antigen-binding domain (e.g., scFv) comprises (a) a heavy chain variable region CDR1 comprising the amino acid sequence of SEQ ID NO:411 or conservative modifications thereof; (b) a heavy chain variable region CDR2 comprising the amino acid sequence of SEQ ID NO:412 or conservative modifications thereof; (c) a heavy chain variable region CDR3 comprising the amino acid sequence of SEQ ID NO:543 or conservative modifications thereof; (d) a light chain variable region CDR1 comprising the amino acid sequence of SEQ ID NO:544 or conservative modifications thereof; (e) a light chain variable region CDR2 comprising the amino acid sequence of SEQ ID NO:448 or conservative modifications thereof; and (f) a light chain variable region CDR3 comprising the amino acid sequence of SEQ ID NO:545 or conservative modifications thereof.

In certain embodiments, the extracellular antigen-binding domain (e.g., scFv) comprises (a) a heavy chain variable region CDR1 comprising the amino acid sequence of SEQ ID NO:372 or conservative modifications thereof; (b) a heavy chain variable region CDR2 comprising the amino acid sequence of SEQ ID NO:475 or conservative modifications thereof; (c) a heavy chain variable region CDR3 comprising the amino acid sequence of SEQ ID NO:570 or conservative modifications thereof; (d) a light chain variable region CDR1 comprising the amino acid sequence of SEQ ID NO:571 or conservative modifications thereof; (e) a light chain variable region CDR2 comprising the amino acid sequence of SEQ ID NO:572 or conservative modifications thereof; and (f) a light chain variable region CDR3 comprising the amino acid sequence of SEQ ID NO:573 or conservative modifications thereof.

In certain embodiments, the extracellular antigen-binding domain (e.g., scFv) comprises (a) a heavy chain variable region CDR1 comprising amino acids having the sequence set forth in SEQ ID NO:923 or conservative modifications thereof; (b) a heavy chain variable region CDR2 comprising amino acids having the sequence set forth in SEQ ID NO:924 or conservative modifications thereof; (c) a heavy chain variable region CDR3 comprising amino acids having the sequence set forth in SEQ ID NO:925 or conservative modifications thereof; (d) a light chain variable region CDR1 comprising amino acids having the sequence set forth in SEQ ID NO:926 or conservative modifications thereof; (e) a light chain variable region CDR2 comprising amino acids having the sequence set forth in SEQ ID NO:927 or conservative modifications thereof; and (f) and a light chain variable region CDR3 comprising amino acids having the sequence set forth in SEQ ID NO:928 or conservative modifications thereof.

In certain embodiments, the extracellular antigen-binding domain (e.g., scFv) comprises (a) a heavy chain variable region CDR1 comprising amino acids having the sequence set forth in SEQ ID NO:929 or conservative modifications thereof; (b) a heavy chain variable region CDR2 comprising amino acids having the sequence set forth in SEQ ID NO:930 or conservative modifications thereof; (c) a heavy chain variable region CDR3 comprising amino acids having the sequence set forth in SEQ ID NO:931 or conservative modifications thereof; (d) a light chain variable region CDR1 comprising amino acids having the sequence set forth in SEQ ID NO:932 or conservative modifications thereof; (e) a light chain variable region CDR2 comprising amino acids having the sequence set forth in SEQ ID NO:933 or conservative modifications thereof, and (f) a light chain variable region CDR3 comprising amino acids having the sequence set forth in SEQ ID NO:934 or conservative modifications thereof.

In certain embodiments, a presently disclosed anti-FcRL5 antibody or antigen-binding fragment thereof comprises: (a) a heavy chain variable region CDR1 comprising the amino acid sequence of SEQ ID NO:440 or conservative modifications thereof; (b) a heavy chain variable region CDR2 comprising the amino acid sequence of SEQ ID NO:441 or conservative modifications thereof; (c) a heavy chain variable region CDR3 comprising the amino acid sequence of SEQ ID NO:442 or conservative modifications thereof; (d) a light chain variable region CDR1 comprising the amino acid sequence of SEQ ID NO:329 or conservative modifications thereof; (e) a light chain variable region CDR2 comprising the amino acid sequence of SEQ ID NO:330 or conservative modifications thereof; and (f) a light chain variable region CDR3 comprising the amino acid sequence of SEQ ID NO:443 or conservative modifications thereof.

In certain embodiments, a presently disclosed anti-FcRL5 antibody or antigen-binding fragment thereof comprises: (a) a heavy chain variable region CDR1 comprising the amino acid sequence of SEQ ID NO:309 or conservative modifications thereof; (b) a heavy chain variable region CDR2 comprising the amino acid sequence of SEQ ID NO:310 or conservative modifications thereof; (c) a heavy chain variable region CDR3 comprising the amino acid sequence of SEQ ID NO:489 or conservative modifications thereof; (d) a light chain variable region CDR1 comprising the amino acid sequence of SEQ ID NO:490 or conservative modifications thereof; (e) a light chain variable region CDR2 comprising the amino acid sequence of SEQ ID NO:313 or conservative modifications thereof; and (f) a light chain variable region CDR3 comprising the amino acid sequence of SEQ ID NO:491 or conservative modifications thereof.

As used herein, the terms “conservative sequence modifications” and “conservative modifications” refers to amino acid modifications that do not significantly affect or alter the binding characteristics of the presently disclosed CAR (e.g., the extracellular antigen-binding domain) comprising the amino acid sequence. Such conservative modifications include amino acid substitutions, additions and deletions. Modifications can be introduced into the human scFv of the presently disclosed subject matter by standard techniques known in the art, such as site-directed mutagenesis and PCR-mediated mutagenesis. Amino acids can be classified into groups according to their physicochemical properties such as charge and polarity.

Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays 10 described herein. In certain embodiments, no more than one, no more than two, no more than three, no more than four, no more than five residues within a specified sequence or a CDR region are altered. Exemplary conservative amino acid substitutions are shown in Table 231.

TABLE 231

| Original | Exemplary conservative |
|---|---|
| Residue | amino acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

In certain non-limiting embodiments, an extracellular antigen-binding domain of the CAR can comprise a linker connecting the heavy chain variable region and light chain variable region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). Non-limiting examples of peptide linkers are disclosed in Shen et al., Anal. Chem. 80(6):1910-1917 (2008). In one non-limiting example, the linker is a G4S linker that comprises amino acids having the sequence set forth in SEQ ID NO:897. In certain embodiments, the nucleotide sequence encoding the amino acid sequence of SEQ ID NO:897 is set forth in SEQ ID NO:898. In one non-limiting example, the linker comprises amino acids having the sequence set forth in SEQ ID NO:307. In certain embodiments, the nucleotide sequence encoding the amino acid sequence of SEQ ID NO:307 is set forth in SEQ ID NO:305.

In certain embodiments, the linker comprises amino acids having the sequence set forth in SEQ ID NO:901 as provided below.

[SEQ ID NO: 901]
GGGGS.

In certain embodiments, the the linker comprises amino acids having the sequence set forth in

[SEQ ID NO: 902]
SGGSGGS.

In certain embodiments, the the linker comprises amino acids having the sequence set forth in SEQ ID NO:903 as provided below.

[SEQ ID NO: 903]
GGGGSGGGS.

In certain embodiments, the the linker comprises amino acids having the sequence set forth in SEQ ID NO:904 as provided below.

[SEQ ID NO: 904]
GGGGSGGGGS.

In certain embodiments, the the linker comprises amino acids having the sequence set forth in SEQ ID NO:905 as provided below.

[SEQ ID NO: 905]

GGGGSGGGGSGGGGGGGS.

In certain embodiments, the the linker comprises amino acids having the sequence set forth in SEQ ID NO:906 as provided below.

[SEQ ID NO: 906]

GGGGSGGGGSGGGGSGGGGS.

In certain embodiments, the the linker comprises amino acids having the sequence set forth in SEQ ID NO:907 as provided below.

[SEQ ID NO: 907]

GGGGSGGGGSGGGGSGGGGSGGGGS.

In certain embodiments, the the linker comprises amino acids having the sequence set forth in 10 SEQ ID NO:908 as provided below.

[SEQ ID NO: 908]

GGGGSGGGGSGGGGSGGGGSGGGGSGGGGS.

In certain embodiments, the the linker comprises amino acids having the sequence set forth in SEQ ID NO:909 as provided below.

[SEQ ID NO: 909]

GGGGSGGGGSGGGGSGGGGSGGGGSGGGGSGGGGS.

In certain embodiments, the the linker comprises amino acids having the sequence set forth in SEQ ID NO:910 as provided below.

[SEQ ID NO: 910]

EPKSCDKTHTCPPCP.

In certain embodiments, the the linker comprises amino acids having the sequence set forth in SEQ ID NO:911 as provided below.

[SEQ ID NO: 911]

GGGGSGGGSEPKSCDKTHTCPPCP.

In certain embodiments, the the linker comprises amino acids having the sequence set forth in SEQ ID NO:912 as provided below.

[SEQ ID NO: 912]
ELKTPLGDTTHTCPRCPEPKSCDTPPPCPRCPEPKSCDTPPPCPRCPEPK
SCDTPPPCPRCP.

In certain embodiments, the the linker comprises amino acids having the sequence set forth in SEQ ID NO:913 as provided below.

[SEQ ID NO: 913]

GSGSGS.

In certain embodiments, the the linker comprises amino acids having the sequence set forth in SEQ ID NO:914 as provided below.

[SEQ ID NO: 914]

AAA.

In certain embodiments, the extracellular antigen-binding domain (e.g., scFv) comprises a heavy chain variable region, a light chain variable region and a linker peptide between the heavy chain variable region and the light chain variable region. Non-limiting examples of extracellular antigen-binding domains, e.g., scFvs, of the present disclosure that comprise a heavy chain variable region, a light chain variable region and a linker peptide are disclosed in Tables 77-152. For example, and not by way of limitation, the extracellular antigen-binding domain comprising a heavy chain variable region, a light chain variable region and a linker peptide of the present disclosure comprises an amino acid sequence selected from the group consisting of SEQ ID NO:594, SEQ ID NO:596, SEQ ID NO:598, SEQ ID NO:600, SEQ ID NO:602, SEQ ID NO:604, SEQ ID NO:606, SEQ ID NO:608, SEQ ID NO:610, SEQ ID NO:612, SEQ ID NO:614, SEQ ID NO:616, SEQ ID NO:618, SEQ ID NO:620, SEQ ID NO:622, SEQ ID NO:624, SEQ ID NO:626, SEQ ID NO:628, SEQ ID NO:630, SEQ ID NO:632, SEQ ID NO:634, SEQ ID NO:636, SEQ ID NO:638, SEQ ID NO:640, SEQ ID NO:642, SEQ ID NO:644, SEQ ID NO:646, SEQ ID NO:648, SEQ ID NO:650, SEQ ID NO:652, SEQ ID NO:654, SEQ ID NO:656, SEQ ID NO:658, SEQ ID NO:660, SEQ ID NO:662, SEQ ID NO:664, SEQ ID NO:666, SEQ ID NO:668, SEQ ID NO:670, SEQ ID NO:672, SEQ ID NO:674, SEQ ID NO:676, SEQ ID NO:678, SEQ ID NO:680, SEQ ID NO:682, SEQ ID NO:684, SEQ ID NO:686, SEQ ID NO:688, SEQ ID NO:690, SEQ ID NO:692, SEQ ID NO:694, SEQ ID NO:696, SEQ ID NO:698, SEQ ID NO:700, SEQ ID NO:702, SEQ ID NO:704, SEQ ID NO:706, SEQ ID NO:708, SEQ ID NO:710, SEQ ID NO:712, SEQ ID NO:714, SEQ ID NO:716, SEQ ID NO:718, SEQ ID NO:720, SEQ ID NO:722, SEQ ID NO:724, SEQ ID NO:726, SEQ ID NO:728, SEQ ID NO:730, SEQ ID NO:732, SEQ ID NO:734, SEQ ID NO:736, SEQ ID NO:738, SEQ ID NO:740, SEQ ID NO:742, SEQ ID NO:744 and conservative modifications of (as shown in Tables 77-152).

In certain embodiments, the extracellular antigen-binding domain (e.g., scFv) having a heavy chain variable region, a light chain variable region and a linker peptide comprises the amino acid sequence of SEQ ID NO:650 or conservative modifications of.

In certain embodiments, the extracellular antigen-binding domain (e.g., scFv) having a heavy chain variable region, a light chain variable region and a linker peptide comprises the amino acid sequence of SEQ ID NO:664 or conservative modifications of.

In certain embodiments, the extracellular antigen-binding domain (e.g., scFv) having a heavy chain variable region, a light chain variable region and a linker peptide comprises the amino acid sequence of SEQ ID NO:678 or conservative modifications of.

In certain embodiments, the extracellular antigen-binding domain (e.g., scFv) having a heavy chain variable region, a light chain variable region and a linker peptide comprises the amino acid sequence of SEQ ID NO:700 or conservative modifications of.

In certain embodiments, the extracellular antigen-binding domain (e.g., scFv) having a heavy chain variable region, a light chain variable region and a linker peptide comprises the amino acid sequence of SEQ ID NO:702 or conservative modifications of.

In certain embodiments, the extracellular antigen-binding domain (e.g., scFv) having a heavy chain variable region, a light chain variable region and a linker peptide comprises the amino acid sequence of SEQ ID NO:710 or conservative modifications of.

In certain embodiments, the extracellular antigen-binding domain (e.g., scFv) having a heavy chain variable region, a light chain variable region and a linker peptide comprises the amino acid sequence of SEQ ID NO:726 or conservative modifications of.

In certain embodiments, the extracellular antigen-binding domain (e.g., scFv) having a heavy chain variable region, a light chain variable region and a linker peptide comprises the amino acid sequence of SEQ ID NO:650 or conservative modifications of.

In certain embodiments, the extracellular antigen-binding domain (e.g., scFv) having a heavy chain variable region, a light chain variable region and a linker peptide comprises the amino acid sequence of SEQ ID NO:678 or conservative modifications of.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In non-limiting examples, the signal peptide is covalently joined to the 5′ terminus of the extracellular antigen-binding domain. In certain embodiments, the signal peptide comprises a CD8 polypeptide comprising amino acids having the sequence set forth in SEQ ID NO:26 as provided below.

[SEQ ID NO: 935]

MALPVTALLLPLALLLHAAR

The nucleotide sequence encoding the amino acid sequence of SEQ ID NO:935 is set forth in SEQ ID NO:936, which is provided below:

[SEQ ID NO: 936]
ATGGCTCTCCCAGTGACTGCCCTACTGCTTCCCCTAGCGCTTCTCCTGCA
TGCAGCTCGT

In another embodiment, the signal peptide comprises amino acids having the sequence set forth in SEQ ID NO:937 as provided below.

[SEQ ID NO: 937]

METDTLLLWVLLLWVPGSTG

The nucleotide sequence encoding the amino acid sequence of SEQ ID NO:937 is set forth in SEQ ID NO:938, which is provided below:

[SEQ ID NO: 938]
ATGGAAACCGACACCCTGCTGCTGTGGGTGCTGCTGCTGTGGGTGCCAGG
ATCCACAGGA

In certain embodiments, the extracellular antigen-binding domain, e.g., the human scFv, comprises a heavy chain variable region, a light chain variable region, a linker peptide between the heavy chain variable region and the light chain variable region, and an His-tag and an HA-tag. In certain embodiments, the amino acid sequence of the His-tag and HA-tag comprises the amino acid sequence of SEQ ID NO:308. The nucleotide sequence encoding SEQ ID NO: 308 is SEQ ID NO: 306.

In certain embodiments, the extracellular antigen-binding domain, e.g., the human scFv, binds to a human FcRL5 polypeptide comprising the amino acid sequence set forth in SEQ ID NO: 899. In certain embodiments, the extracellular antigen-binding domain, e.g., the human scFv, binds to an epitope in domain 9 (e.g., amino acids 754-835 of SEQ ID NO:899). In certain embodiments, the extracellular antigen-binding domain, e.g., the human scFv, binds to an epitope in domain 8 (e.g., amino acids 658-731 of SEQ ID NO:899). In certain embodiments, the extracellular antigen-binding domain, e.g., the human scFv, binds to an epitope within domain 9 comprising amino acids 829-840 of SEQ ID NO:899. In certain embodiments, the extracellular antigen-binding domain, e.g., the human scFv, binds to an epitope within domain 8 comprising amino acids 657-667 of SEQ ID NO:899. For example, and not by way of limitation, the extracellular antigen-binding domain, e.g., the human scFv, binds to an epitope comprising the amino acid sequence RSETVTLYITGL (SEQ ID NO:964). In certain embodiments, In certain embodiments, an antibody or an antigen-binding fragment thereof of the present disclosure binds to an epitope comprising the amino acid sequence SRPILTFRAPR (SEQ ID NO:965).

### Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP_006130 (SEQ ID NO:939), or fragments thereof, and/or may optionally comprise up to one or up to two or up to three conservative amino acid substitutions. In non-limiting embodiments, the CD28 polypeptide can have an amino acid sequence that is a consecutive portion of SEQ ID NO:939 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 150 to 200, or 200 to 220 of SEQ ID NO:939. In certain embodiments, the CAR of the presently disclosed subject matter comprises a transmembrane domain comprising a CD28 polypeptide, and an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO:939.

SEQ ID NO:939 is provided below:

| [SEQ ID NO: 939] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain and the intracellular domain (e.g., the co-stimulatory signaling region) of the presently disclosed CAR (amino acids 114 to 220 of SEQ ID NO:939) comprises nucleic acids having the sequence set forth in SEQ ID NO:940 as provided below.

[SEQ ID NO: 940]
ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCC.

In certain embodiments, the transmembrane domain of a presently disclosed CAR comprises a CD8 polypeptide. The CD8 polypeptide can have an amino acid sequence that is at least about 85%, about 90%, about 95%, about 96%, about 97%, about 98%, about 99% or 100% homologous to the sequence having a NCBI Reference No: AAH25715 (SEQ ID NO:960), or fragments thereof, and/or may optionally comprise up to one or up to two or up to three conservative amino acid substitutions. In non-limiting embodiments, the CD8 polypeptide can have an amino acid sequence that is a consecutive portion of SEQ ID NO:960 which is at least 20, or at least 30, or at least 40, or at least 50, or at least 70, or at least 100, or at least 150, or at least 200 and up to 235 amino acids in length. Alternatively or additionally, in non-limiting various embodiments, the CD8 polypeptide has an amino acid sequence of amino acids 1 to 235, 1 to 50, 50 to 100, 100 to 150, 150 to 200, 130 to 210, or 200 to 235 of SEQ ID NO:960. In certain embodiments, the CAR of the presently disclosed subject matter comprises a transmembrane domain comprising a CD8 polypeptide. In certain embodiments, the CD8 polypeptide comprised in the transmembrane domain has an amino acid sequence of amino acids 137 to 207 of SEQ ID NO:960.

SEQ ID NO:960 is provided below:

| [SEQ ID NO: 960] | |
|---|---|
| 1 | malpvtalll plalllhaar psqfrvspld rtwnlgetve lkcqvllsnp tsgcswlfqp |
| 61 | rgaaasptfl lylsqnkpka aegldtqrfs gkrlgdtfvl tlsdfrrene gcyfcsalsn |
| 121 | simyfshfvp vflpakpttt paprpptpap tiasqplslr peacrpaagg avhtrgldfa |
| 181 | cdiyiwapla gtcgvlllsl vitlycnhrn rrrvckcprp vvksgdkpsl saryv |

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain and the intracellular domain (e.g., the co-stimulatory signaling region) of the presently disclosed CAR (amino acids 137 to 207 of SEQ ID NO:960) comprises nucleic acids having the sequence set forth in SEQ ID NO:961 as provided below.

[SEQ ID NO: 961]
CCCACCACGACGCCAGCGCCGCGACCACCAACCCCGGCGCCCACGATCG
CGTCGCAGCCCCTGTCCCTGCGCCCAGAGGCGTGCCGGCCAGCGGCGGG
GGGCGCAGTGCACACGAGGGGGCTGGACTTCGCCTGTGATATCTACATC
TGGGCGCCCCTGGCCGGGACTTGTGGGGTCCTTCTCCTGTCACTGGTTA
TCACCCTTTACTGCAAC

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3.

### Intracellular Domain of a CAR

In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises three ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence set forth in SEQ ID NO:941, or fragments thereof, and/or may optionally comprise up to one or up to two or up to three conservative amino acid substitutions. In non-limiting embodiments, the CD3ζ polypeptide can have an amino acid sequence that is a consecutive portion of SEQ ID NO:941 which is at least 20, or at least 30, or at least 40, or at least 50, and up to 163 amino acids in length. Alternatively or additionally, in non-limiting various embodiments, the CD3ζ polypeptide has an amino acid sequence of amino acids 1 to 163, 1 to 50, 50 to 100, 100 to 150, or 150 to 163 of SEQ ID NO:941. In certain embodiments, the CD3ζ polypeptide comprised in the intracellular domain of a presently disclosed CAR has an amino acid sequence of amino acids 52 to 163 of SEQ ID NO: 941.

SEQ ID NO: 941 is provided below:

| [SEQ ID NO: 941] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP RRKNPQEGLY NELQKDKMAE |
| 121 | AYSEIGMKGE RRRGKGHDGL YQGLSTATKD TYDALHMQAL PPR |

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide comprised in the intracellular domain of a presently disclosed CARs (amino acids 52 to 163 of SEQ ID NO: 941) comprises nucleic acids having the sequence set forth in SEQ ID NO:942 as provided below.

[SEQ ID NO: 942]
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD137”) for providing an intracellular signal that in combination with a CAR signal induces an effector cell function of the CAR+ T cell. CARs comprising an intracellular domain that comprises a co-stimulatory signaling region comprising 4-1BB, ICOS or DAP-10 are disclosed in U.S. Pat. No. 7,446,190 (e.g., the nucleotide sequence encoding 4-1BB is set forth in SEQ ID NO:15, the nucleotide sequence encoding ICOS is set forth in SEQ ID NO:16, and the nucleotide sequence encoding DAP-10 is set forth in SEQ ID NO:17 in U.S. Pat. No. 7,446,190), which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules:CD28 and 4-1BB or CD28 and OX40. 4-1BB can act as a tumor necrosis factor (TNF) ligand and have stimulatory activity. The 4-1BB polypeptide can have an amino acid sequence that is at least about 85%, about 90%, about 95%, about 96%, about 97%, about 98%, about 99% or 100% homologous to the sequence having a NCBI Reference No: P41273 or NP_001552 (SEQ ID NO:943) or fragments thereof, and/or may optionally comprise up to one or up to two or up to three conservative amino acid substitutions. In certain embodiments, the 4-1BB polypeptide comprised in the intracellular domain of a presently disclosed CAR has an amino acid sequence of amino acids 214 to 255 of SEQ ID NO: 943. SEQ ID NO:943 is provided below:

| [SEQ ID NO: 943] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide comprised in the intracellular domain of a presently disclosed CARs (amino acids 214 to 255 of SEQ ID NO: 943) comprises nucleic acids having the sequence set forth in SEQ ID NO: 962 as provided below.

[SEQ ID NO: 962]
AAACGGGGCAGAAAGAAGCTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a NCBI Reference No: P43489 or NP_003318 (SEQ ID NO:944), or fragments thereof, and/or may optionally comprise up to one or up to two or up to three conservative amino acid substitutions. SEQ ID NO:944 is provided below:

| [SEQ ID NO: 944] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO:945) or fragments thereof, and/or may optionally comprise up to one or up to two or up to three conservative amino acid substitutions.

SEQ ID NO:945 is provided below:

| [SEQ ID NO: 945] | |
|---|---|
| 1 | MKSGLWYFFL FCLRIKVLTG EINGSANYEM FIFHNGGVQI LCKYPDIVQQ FKMQLLKGGQ |
| 61 | ILCDLTKTKG SGNTVSIKSL KFCHSQLSNN SVSFFLYNLD HSHANYYFCN LSIFDPPPFK |
| 121 | VTLTGGYLHI YESQLCCQLK FWLPIGCAAF VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

CTLA-4 is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion, enhanced the endogenous anti-tumor response albeit, in the clinical setting, with significant unforeseen toxicities.

CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif able to bind PI3K, PP2A and SHP-2 and one proline-rich motif able to bind SH3 containing proteins. One role of CTLA-4 in inhibiting T cell responses seem to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO:946) (homology herein may be determined using standard software such as BLAST or FASTA) or fragments thereof, and/or may optionally comprise up to one or up to two or up to three conservative amino acid substitutions.

SEQ ID NO:946 is provided below:

| [SEQ ID NO: 946] | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN |

In accordance with the presently disclosed subject matter, a “CTLA-4 nucleic acid molecule” refers to a polynucleotide encoding a CTLA-4 polypeptide.

PD-1 is a negative immune regulator of activated T cells upon engagement with its corresponding ligands PD-L1 and PD-L2 expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, that PD-1 negatively regulates TCR signals. SHP-I and SHP-2 phosphatases bind to the cytoplasmic tail of PD-1 upon ligand binding. Upregulation of PD-L1 is one mechanism tumor cells may evade the host immune system. In pre-clinical and clinical trials, PD-1 blockade by antagonistic antibodies induced anti-tumor responses mediated through the host endogenous immune system.

In accordance with the presently disclosed subject matter, a PD-1 polypeptide can have an amino acid sequence that is at least about 85%, about 90%, about 95%, about 96%, about 97%, about 98%, about 99% or about 100% homologous to NCBI Reference No: NP_005009.2 (SEQ ID NO:947) or fragments thereof, and/or may optionally comprise up to one or up to two or up to three conservative amino acid substitutions.

SEQ ID NO:947 is provided below:

| [SEQ ID NO: 947] | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

In accordance with the presently disclosed subject matter, a “PD-1 nucleic acid molecule” refers to a polynucleotide encoding a PD-1 polypeptide.

Lymphocyte-activation protein 3 (LAG-3) is a negative immune regulator of immune cells. LAG-3 belongs to the immunoglobulin (lg) superfamily and contains 4 extracellular Ig-like domains. The LAG3 gene contains 8 exons. The sequence data, exon/intron organization, and chromosomal localization all indicate a close relationship of LAG3 to CD4. LAG3 has also been designated CD223 (cluster of differentiation 223).

In accordance with the the presently disclosed subject matter, a LAG-3 polypeptide can have an amino acid sequence that is at least about 85%, about 90%, about 95%, about 96%, about 97%, about 98%, about 99% or about 100% homologous to UniProtKB/Swiss-Prot Ref. No.: P18627.5 (SEQ ID NO:948) or fragments thereof, and/or may optionally comprise up to one or up to two or up to three conservative amino acid substitutions.

SEQ ID NO:948 is provided below:

| [SEQ ID NO: 948] | |
|---|---|
| 1 | MWEAQFLGLL FLQPLWVAPV KPLQPGAEVP VVWAQEGAPA QLPCSPTIPL QDLSLLRRAG |
| 61 | VTWQHQPDSG PPAAAPGHPL APGPHPAAPS SWGPRPRRYT VLSVGPGGLR SGRLPLQPRV |
| 121 | QLDERGRQRG DFSLWLRPAR RADAGEYRAA VHLRDRALSC RLRLRLGQAS MTASPPGSLR |
| 181 | ASDWVILNCS FSRPDRPASV HWFRNRGQGR VPVRESPHHH LAESFLFLPQ VSPMDSGPWG |
| 241 | CILTYRDGFN VSIMYNLTVL GLEPPTPLTV YAGAGSRVGL PCRLPAGVGT RSFLTAKWTP |
| 301 | PGGGPDLLVT GDNGDFTLRL EDVSQAQAGT YTCHIHLQEQ QLNATVTLAI ITVTPKSFGS |
| 361 | PGSLGKLLCE VTPVSGQERF VWSSLDTPSQ RSFSGPWLEA QEAQLLSQPW QCQLYQGERL |
| 421 | LGAAVYFTEL SSPGAQRSGR APGALPAGHL LLFLILGVLS LLLLVTGAFG FHLWRRQWRP |
| 481 | RRFSALEQGI HPPQAQSKIE ELEQEPEPEP EPEPEPEPEP EPEQL |

In accordance with the presently disclosed subject matter, a “LAG-3 nucleic acid molecule” refers to a polynucleotide encoding a LAG-3 polypeptide.

Natural Killer Cell Receptor 2B4 (2B4) mediates non-MHC restricted cell killing on NK cells and subsets of T cells. To date, the function of 2B4 is still under investigation, with the 2B4-S isoform believed to be an activating receptor, and the 2B4-L isoform believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244). In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref. No.: Q9BZW8.2 (SEQ ID NO:949) or fragments thereof, and/or may optionally comprise up to one or up to two or up to three conservative amino acid substitutions.

SEQ ID NO:949 is provided below:

| [SEQ ID NO: 949] | |
|---|---|
| 1 | MLGQVVTLIL LLLLKVYQGK GCQGSADHVV SISGVPLQLQ PNSIQTKVDS IAWKKLLPSQ |
| 61 | NGFHHILKWE NGSLPSNTSN DRFSFIVKNL SLLIKAAQQQ DSGLYCLEVT SISGKVQTAT |
| 121 | FQVFVFESLL PDKVEKPRLQ GQGKILDRGR CQVALSCLVS RDGNVSYAWY RGSKLIQTAG |
| 181 | NLTYLDEEVD INGTHTYTCN VSNPVSWESH TLNLTQDCQN AHQEFRFWPF LVIIVILSAL |
| 241 | FLGTLACFCV WRRKRKEKQS ETSPKEFLTI YEDVKDLKTR RNHEQEQTFP GGGSTIYSMI |
| 301 | QSQSSAPTSQ EPAYTLYSLI QPSRKSGSRK RNHSPSFNST IYEVIGKSQP KAQNPARLSR |
| 361 | KELENFDVYS |

In accordance with the presently disclosed subject matter, a “2B4 nucleic acid molecule” refers to a polynucleotide encoding a 2B4 polypeptide.

B- and T-lymphocyte attenuator (BTLA) expression is induced during activation of T cells, and BTLA remains expressed on Th1 cells but not Th2 cells. Like PD1 and CTLA4, BTLA interacts with a B7 homolog, B7H4. However, unlike PD-1 and CTLA-4, BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

In accordance with the presently disclosed subject matter, a BTLA polypeptide can have an amino acid sequence that is at least about 85%, about 90%, about 95%, about 96%, about 97%, about 98%, about 99% or about 100% homologous to UniProtKB/Swiss-Prot Ref. No.: Q7Z6A9.3 (SEQ ID NO:950) or fragments thereof, and/or may optionally comprise up to one or up to two or up to three conservative amino acid substitutions.

SEQ ID NO:950 is provided below:

| | [SEQ ID NO: 950] |
|---|---|
| 1 | MKTLPAMLGT GKLFWVFFLI PYLDIWNIHG KESCDVQLYI KRQSEHSILA GDPFELECPV |
| 61 | KYCANRPHVT WCKLNGTTCV KLEDRQTSWK EEKNISFFIL HFEPVLPNDN GSYRCSANFQ |
| 121 | SNLIESHSTT LYVTDVKSAS ERPSKDEMAS RPWLLYRLLP LGGLPLLITT CFCLFCCLRR |
| 181 | HQGKQNELSD TAGREINLVD AHLKSEQTEA STRQNSQVLL SETGIYDNDP DLCFRMQEGS |
| 241 | EVYSNPCLEE NKPGIVYASL NHSVIGPNSR LARNVKEAPT EYASICVRS |

In accordance with the presently disclosed subject matter, a “BTLA nucleic acid molecule” refers to a polynucleotide encoding a BTLA polypeptide.

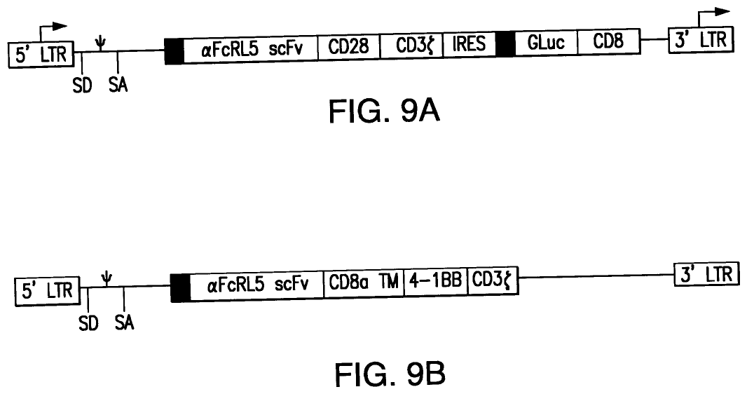

In certain embodiments, the CAR comprises an extracellular antigen-binding region that specifically binds to human FcRL5, a transmembrane domain comprising a CD28 polypeptide, and an intracellular domain comprising a CD3ζ polypeptide and a co-stimulatory signaling region that comprises a CD28 polypeptide, as shown in FIG. 9. As shown in FIG. 9, the CAR also comprises a signal peptide or a leader covalently joined to the 5′ terminus of the extracellular antigen-binding domain. The signal peptide comprises a CD8 polypeptide.

In certain embodiments, the CAR of the presently disclosed subject matter can further comprise an inducible promoter, for expressing nucleic acid sequences in human cells. Promoters for use in expressing CAR genes can be a constitutive promoter, such as ubiquitin C (UbiC) promoter.

The presently disclosed subject matter also provides isolated nucleic acid molecule encoding the FcRL5-targeted CAR described herein or a functional portion thereof. In certain embodiments, the isolated nucleic acid molecule encodes a presently disclosed FcRL5-targeted CAR comprising an scFv that specifically binds to human FcRL5, a transmembrane domain comprising a CD28 polypeptide, and an intracellular domain comprising a CD3 polypeptide and a co-stimulatory signaling region comprising a CD28 polypeptide. In certain embodiments, the scFv is a fully human scFv. In certain embodiments, the scFv is a murine scFv. In certain non-limiting embodiments, the isolated nucleic acid molecule comprises nucleic acids having the sequence set forth in SEQ ID NO:951 provided below:

[SEQ ID NO: 951]
ATGGCTCTCCCAGTGACTGCCCTACTGCTTCCCCTAGCGCTTCTCCTGC
ATGCAGCTCGTGTGAAGCTGCAGGAGTCTGGGGGAGGCTTAGTGCAGCC
TGGAGGGTCCCGGAAACTCTCCTGTGCAGCCTCTGGATTCACTTTCAGT
ATCTTTGGATTGCACTGGGTTCGTCAGGCTCCAGAGAAGGGGCTGGAGT
GGGTCGCATACATTAGTGGTGACAGTAATACCATCTACTATGCAGACAC
AGTGAAGGGCCGATTCACCATCTCCAGAGACAATCCCAAGAACACCCTG
TTCCTGCAAATGACCAGTCTAAGGTCTGAGGACACGGCCATGTATTACT
GTGCAAGAAATAGCTACTATGCTCTGGACTACTGGGGCCAAGGGACCAC
GGTCACCGTCTCCTCAGGTGGAGGTGGATCAGGTGGAGGTGGATCTGGT
GGAGGTGGATCTGACATTGAGCTCACCCAGTCTCCAGCAATCATGTCTG
TATCTCCAGGTGAAAAGGTCACCATGACCTGCAGGGCCAGCTCAAGTGT
CAGTTCCAGTTACTTGCACTGGTACCAGCAGAGGTCAGGTGCCTCCCCC
AAAATCTGGATTTATAGCACATCCAACTTGGCTTCTGGAGTCCCTGCTC
GCTTCAGTGGCAGTGGGACTGGGACCTCTTACTCTCTCACAATCAGCAG
TGTGGAGGCTGAAGATGCTGCCACTTATTACTGCCAGCAGTACAGTGGT
TACCCGTGGACGTTCGGTGGAGGGACCAAGCTGGAGATCGAACAAAAAC
TCATCTCAGAAGAGGATCTGGCGGCCGCAATTGAAGTTATGTATCCTCC
TCCTTACCTAGACAATGAGAAGAGCAATGGAACCATTATCCATGTGAAA
GGGAAACACCTTTGTCCAAGTCCCCTATTTCCCGGACCTTCTAAGCCCT
TTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGG
CTCCTGCACAGTGACTACATGAACATGACTCCCCGCCGCCCCGGGCCCA
CCCGCAAGCATTACCAGCCCTATGCCCCACCACGCGACTTCGCAGCCTA
TCGCTCCAGAGTGAAGTTCAGCAGGAGCGCAGACGCCCCCGCGTACCAG
CAGGGCCAGAACCAGCTCTATAACGAGCTCAATCTAGGACGAAGAGAGG
AGTACGATGTTTTGGACAAGAGACGTGGCCGGGACCCTGAGATGGGGGG
AAAGCCGAGAAGGAAGAACCCTCAGGAAGGCCTGTACAATGAACTGCAG
AAAGATAAGATGGCGGAGGCCTACAGTGAGATTGGGATGAAAGGCGAGC
GCCGGAGGGGCAAGGGGCACGATGGCCTTTACCAGGGTCTCAGTACAGC
CACCAAGGACACCTACGACGCCCTTCACATGCAGGCCCTGCCCCCTCGC
TAA

The isolated nucleic acid molecule having the nucleotide sequence of SEQ ID NO:951 encodes a FcRL-5-targeted CAR comprising a murine scFv that comprises a heavy chain variable region comprising amino acids having the sequence set forth in SEQ ID NO:915, a light chain variable region comprising amino acids having the sequence set forth in SEQ ID NO:917, and a linker having an amino acid sequence of SEQ ID NO:897 positioned between the heavy chain variable region and the light chain variable region, a transmembrane domain comprising a CD28 polypeptide, and an intracellular domain comprising a CD3 polypeptide and a co-stimulatory signaling region comprising a CD28 polypeptide.

In another specific non-limiting example, the isolated nucleic acid molecule comprises nucleic acids having the sequence set forth in SEQ ID NO:952 provided below:

[SEQ ID NO: 952]
ATGGAGACAGACACACTCCTGCTATGGGTACTGCTGCTCTGGGTTCCAG
GTTCCACTGGTGACGTCCAACTGCAGGAGTCAGGGGGAGGCTTAGTGCA
GCCTGGAGGGTCCCGGAAACTCTCCTGTACAGCCTCTGGATTCACTTTC
AGTAGCTTTGGAATGCACTGGGTTCGTCAGGCTCCAGAGAAGGGGCTGG
AGTGGGTCGCATACATTAGTAGTGGCAGTAATAACATCTACTTTGCGGA
CACAGTGAAGGGCCGATTCACCATCTCCAGAGACAATCCCAAGAACACC
CTGTTCCTGCAAATGACCAGTCTAAGGTCTGAGGACACGGCCATGTATT
ACTGTGCAAGATCGGAATACTACGGTAGTAGCCATATGGACTACTGGGG
CCAAGGGACCACGGTCACCGTCTCCTCAGGTGGAGGTGGATCAGGTGGA
GGTGGATCTGGTGGAGGTGGATCTGACATTGAGCTCACCCAGTCTCCAA
AATTCATGTCCACATCAGTAGGAGACAGGGTCAGCGTCACCTGCAAGGC
CAGTCAGAATGTGGGTACTAATGTAGCCTGGTATCAACAGAAACCAGGA
CAATCTCCTAAACCACTGATTTACTCGGCAACCTACCGGAACAGTGGAG
TCCCTGATCGCTTCACAGGCAGTGGATCTGGGACAGATTTCACTCTCAC
CATCACTAACGTGCAGTCTAAAGACTTGGCAGACTATTTCTGTCAACAA
TATAACAGGTATCCGTACACGTCCGGAGGGGGGACCAAGCTGGAGATCG
AACAAAAACTCATCTCAGAAGAGGATCTGGCGGCCGCAATTGAAGTTAT
GTATCCTCCTCCTTACCTAGACAATGAGAAGAGCAATGGAACCATTATC
CATGTGAAAGGGAAACACCTTTGTCCAAGTCCCCTATTTCCCGGACCTT
CTAAGCCCTTTTGGGTGCTGGTGGTGGTTGGTGGAGTCCTGGCTTGCTA
TAGCTTGCTAGTAACAGTGGCCTTTATTATTTTCTGGGTGAGGAGTAAG
AGGAGCAGGCTCCTGCACAGTGACTACATGAACATGACTCCCCGCCGCC
CCGGGCCCACCCGCAAGCATTACCAGCCCTATGCCCCACCACGCGACTT
CGCAGCCTATCGCTCCAGAGTGAAGTTCAGCAGGAGCGCAGACGCCCCC
GCGTACCAGCAGGGCCAGAACCAGCTCTATAACGAGCTCAATCTAGGAC
GAAGAGAGGAGTACGATGTTTTGGACAAGAGACGTGGCCGGGACCCTGA
GATGGGGGGAAAGCCGAGAAGGAAGAACCCTCAGGAAGGCCTGTACAAT
GAACTGCAGAAAGATAAGATGGCGGAGGCCTACAGTGAGATTGGGATGA
AAGGCGAGCGCCGGAGGGGCAAGGGGCACGATGGCCTTTACCAGGGTCT
CAGTACAGCCACCAAGGACACCTACGACGCCCTTCACATGCAGGCCCTG
CCCCCTCGCTAA.

The isolated nucleic acid molecule having the nucleotide sequence of SEQ ID NO:952 encodes a FcRL-5-targeted CAR comprising a murine scFv that comprises a heavy chain variable region comprising amino acids having the sequence set forth in SEQ ID NO:919, a light chain variable region comprising amino acids having the sequence set forth in SEQ ID NO:921, and a linker having an amino acid sequence of SEQ ID NO:897 positioned between the heavy chain variable region and the light chain variable region, a transmembrane domain comprising a CD28 polypeptide, and an intracellular domain comprising a CD3 ζ polypeptide and a co-stimulatory signaling region comprising a CD28 polypeptide.

In certain embodiments, the isolated nucleic acid molecule comprises nucleic acids having the sequence set forth in SEQ ID NO:953 provided below:

[SEQ ID NO: 953]
CCGGTGGTACCTCACCCTTACCGAGTCGGCGACACAGTGTGGGTCCGCC
GACACCAGACTAAGAACCTAGAACCTCGCTGGAAAGGACCTTACACAGT
CCTGCTGACCACCCCCACCGCCCTCAAAGTAGACGGCATCGCAGCTTGG
ATACACGCCGCCCACGTGAAGGCTGCCGACCCCGGGGGTGGACCATCCT
CTAGACTGCCATGGAAACCGATACACTGCTGCTGTGGGTGCTGCTGCTG
TGGGTGCCAGGATCCACAGGCTCCTATGTGCTGACTCAGCCACCCTCAG
TGTCAGTGGCCCCAGGAAAGACGGCCAGGATTACCTGTGGGGGAAACAA
CATTGGAAGTAAAAGTGTGCACTGGTACCAGCAGAAGCCAGGCCAGGCC
CCTGTGCTGGTCATCTATTATGATAGCGACCGGCCCTCAGGGATCCCTG
AGCGATTCTCTGGCTCCAACTCTGGGAACACGGCCACCCTGACCATCAG
CAGGGTCGAAGCCGGGGATGAGGCCGACTATTACTGTCAGGTGTGGGAT
AGTAGTAGTGATTATGTCTTCGGAACTGGGACCAAGGTCACCGTCCTAG
GTTCTAGAGGTGGTGGTGGTAGCGGCGGCGGCGGCTCTGGTGGTGGTGG
ATCCCTCGAGATGGCCGAGGTGCAGCTGGTGGAGACTGGGGGAGGCTTG
GTCAAGCCTGGAGGGTCCCTGAGACTCTCCTGTGCAGCCTCTGGATTCA
CCGTCAGTGACTACTACATGAGCTGGATCCGCCAGGCTCCAGGGAAGGG
CCTGGAGTGGATTTCATACATTAGTGGTAGTGGTAATAGCATATACTAC
GCAGACTCTGTGAAGGGCCGATTCACCATCTCCAGGGACAACGCCAAGA
ACTCACTGGATCTGCAAATGACCAGCCTGAGAGCCGAGGACACGGCCGT
ATATTACTGTGCGCGCTCTACTAAATTCGATTACTGGGGTCAAGGTACT
CTGGTGACCGTCTCCTCAGCGGCCGCACCCACCACGACGCCAGCGCCGC
GACCACCAACCCCGGCGCCCACGATCGCGTCGCAGCCCCTGTCCCTGCG
CCCAGAGGCGTGCCGGCCAGCGGCGGGGGGCGCAGTGCACACGAGGGGG
CTGGACTTCGCCTGTGATATCTACATCTGGGCGCCCCTGGCCGGGACTT
GTGGGGTCCTTCTCCTGTCACTGGTTATCACCCTTTACTGCAACAAACG
GGGCAGAAAGAAGCTCCTGTATATATTCAAACAACCATTTATGAGACCA
GTACAAACTACTCAAGAGGAAGATGGCTGTAGCTGCCGATTTCCAGAAG
AAGAAGAAGGAGGATGTGAACTGAGAGTGAAGTTCAGCAGGAGCGCAGA
GCCCCCCGCGTACCAGCAGGGCCAGAACCAGCTCTATAACGAGCTCAAT
CTAGGACGAAGAGAGGAGTACGATGTTTTGGACAAGAGACGTGGCCGGG
ACCCTGAGATGGGGGGAAAGCCGAGAAGGAAGAACCCTCAGGAAGGCCT
GTACAATGAACTGCAGAAAGATAAGATGGCGGAGGCCTACAGTGAGATT
GGGATGAAAGGCGAGCGCCGGAGGGGCAAGGGGCACGATGGCCTTTACC
AGGGTCTCAGTACAGCCACCAAGGACACCTACGACGCCCTTCACATGCA
GGCCCTGCCCCCTCGCTAACAGCCACTCGAGGATCCGGATTAGTCCAAT
TTGTTAAAGACAGGATATCAGTGGTCCAGGCTCTAGTTTTGACTCAACA
ATATCACCAGCTGAAGCCTATAGAGTACGAGCCATAGATAAAATAAAAG
ATTTTATTTAGTCTCCAGAAAAAGGGGGGAATGAAAGACCCCACCTGTA
GGTTTGGCAAGCTAGCTTAAGTAACGCCATTTTGCAAGGCATGGAAAAA
TACATAACTGAGAATAGAGAAGTTCAGATCAAGGTCAGGAACAGATGGA
ACAGCTGAATATGGGCCAAACAGGATATCTGTGGTAAGCAGTTCCTGCC
CCGGCTCAGGGCCAAGAACAGATGGAACAGCTGAATATGGGCCAAACAG
GATATCTGTGGTAAGCAGTTCCTGCCCCGGCTCAGGGCCAAGAACAGAT
GGTCCCCAGATGCGGTCCAGCCCTCAGCAGTTTCTAGAGAACCATCAGA
TGTTTCCAGGGTGCCCCAAGGACCTGAAATGACCCTGTGCCTTATTTGA
ACTAACCAATCAGTTCGCTTCTCGCTTCTGTTCGCGCGCTTCTGCTCCC
CGAGCTCAATAAAAGAGCCCACAACCCCTCACTCGGGGCGCCAGTCCTC
CGATTGACTGAGTCGCCCGGGTACCCGTGTATCCAATAAACCCTCTTGC
AGTTGCATCCGACTTGTGGTCTCGCTGTTCCTTGGGAGGGTCTCCTCTG
AGTGATTGACTACCCGTCAGCGGGGGTCTTTCACACATGCAGCATGTAT
CAAAATTAATTTGGTTTTTTTTCTTAAGTATTTACATTAAATGGCCATA
GTACTTAAAGTTACATTGGCTTCCTTGAAATAAACATGGAGTATTCAGA
ATGTGTCATAAATATTTCTAATTTTAAGATAGTATCTCCATTGGCTTTC
TACTTTTTCTTTTATTTTTTTTTGTCCTCTGTCTTCCATTTGTTGTTGT
TGTTGTTTGTTTGTTTGTTTGTTGGTTGGTTGGTTAATTTTTTTTTAAA
GATCCTACACTATAGTTCAAGCTAGACTATTAGCTACTCTGTAACCCAG
GGTGACCTTGAAGTCATGGGTAGCCTGCTGTTTTAGCCTTCCCACATCT
AAGATTACAGGTATGAGCTATCATTTTTGGTATATTGATTGATTGATTG
ATTGATGTGTGTGTGTGTGATTGTGTTTGTGTGTGTGACTGTGAAAATG
TGTGTATGGGTGTGTGTGAATGTGTGTATGTATGTGTGTGTGTGAGTGT
GTGTGTGTGTGTGTGCATGTGTGTGTGTGTGACTGTGTCTATGTGTATG
ACTGTGTGTGTGTGTGTGTGTGTGTGTGTGTGTGTGTGTGTGTGTGTGT
TGTGAAAAAATATTCTATGGTAGTGAGAGCCAACGCTCCGGCTCAGGTG
TCAGGTTGGTTTTTGAGACAGAGTCTTTCACTTAGCTTGGAATTCACTG
GCCGTCGTTTTACAACGTCGTGACTGGGAAAACCCTGGCGTTACCCAAC
TTAATCGCCTTGCAGCACATCCCCCTTTCGCCAGCTGGCGTAATAGCGA
AGAGGCCCGCACCGATCGCCCTTCCCAACAGTTGCGCAGCCTGAATGGC
GAATGGCGCCTGATGCGGTATTTTCTCCTTACGCATCTGTGCGGTATTT
CACACCGCATATGGTGCACTCTCAGTACAATCTGCTCTGATGCCGCATA
GTTAAGCCAGCCCCGACACCCGCCAACACCCGCTGACGCGCCCTGACGG
GCTTGTCTGCTCCCGGCATCCGCTTACAGACAAGCTGTGACCGTCTCCG
GGAGCTGCATGTGTCAGAGGTTTTCACCGTCATCACCGAAACGCGCGAT
GACGAAAGGGCCTCGTGATACGCCTATTTTTATAGGTTAATGTCATGAT
AATAATGGTTTCTTAGACGTCAGGTGGCACTTTTCGGGGAAATGTGCGC
GGAACCCCTATTTGTTTATTTTTCTAAATACATTCAAATATGTATCCGC
TCATGAGACAATAACCCTGATAAATGCTTCAATAATATTGAAAAAGGAA
GAGTATGAGTATTCAACATTTCCGTGTCGCCCTTATTCCCTTTTTTGCG
GCATTTTGCCTTCCTGTTTTTGCTCACCCAGAAACGCTGGTGAAAGTAA
AAGATGCTGAAGATCAGTTGGGTGCACGAGTGGGTTACATCGAACTGGA
TCTCAACAGCGGTAAGATCCTTGAGAGTTTTCGCCCCGAAGAACGTTTT
CCAATGATGAGCACTTTTAAAGTTCTGCTATGTGGCGCGGTATTATCCC
GTATTGACGCCGGGCAAGAGCAACTCGGTCGCCGCATACACTATTCTCA
GAATGACTTGGTTGAGTACTCACCAGTCACAGAAAAGCATCTTACGGAT
GGCATGACAGTAAGAGAATTATGCAGTGCTGCCATAACCATGAGTGATA
ACACTGCGGCCAACTTACTTCTGACAACGATCGGAGGACCGAAGGAGCT
AACCGCTTTTTTGCACAACATGGGGGATCATGTAACTCGCCTTGATCGT
TGGGAACCGGAGCTGAATGAAGCCATACCAAACGACGAGCGTGACACCA
CGATGCCTGTAGCAATGGCAACAACGTTGCGCAAACTATTAACTGGCGA
ACTACTTACTCTAGCTTCCCGGCAACAATTAATAGACTGGATGGAGGCG
GATAAAGTTGCAGGACCACTTCTGCGCTCGGCCCTTCCGGCTGGCTGGT
TTATTGCTGATAAATCTGGAGCCGGTGAGCGTGGGTCTCGCGGTATCAT
TGCAGCACTGGGGCCAGATGGTAAGCCCTCCCGTATCGTAGTTATCTAC
ACGACGGGGAGTCAGGCAACTATGGATGAACGAAATAGACAGATCGCTG
AGATAGGTGCCTCACTGATTAAGCATTGGTAACTGTCAGACCAAGTTTA
CTCATATATACTTTAGATTGATTTAAAACTTCATTTTTAATTTAAAAGG
ATCTAGGTGAAGATCCTTTTTGATAATCTCATGACCAAAATCCCTTAAC
GTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACA
AAAAAACCACCGCTACCAGCGGTGGTTTGTTTGCCGGATCAAGAGCTAC
CAACTCTTTTTCCGAAGGTAACTGGCTTCAGCAGAGCGCAGATACCAAA
TACTGTCCTTCTAGTGTAGCCGTAGTTAGGCCACCACTTCAAGAACTCT
GTAGCACCGCCTACATACCTCGCTCTGCTAATCCTGTTACCAGTGGCTG
CTGCCAGTGGCGATAAGTCGTGTCTTACCGGGTTGGACTCAAGACGATA
GTTACCGGATAAGGCGCAGCGGTCGGGCTGAACGGGGGGTTCGTGCACA
CAGCCCAGCTTGGAGCGAACGACCTACACCGAACTGAGATACCTACAGC
GTGAGCATTGAGAAAGCGCCACGCTTCCCGAAGGGAGAAAGGCGGACAG
GTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCT
ATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGG
ATAACCGTATTACCGCCTTTGAGTGAGCTGATACCGCTCGCCGCAGCCG
AACGACCGAGCGCAGCGAGTCAGTGAGCGAGGAAGCGGAAGAGCGCCCA
ATACGCAAACCGCCTCTCCCCGCGCGTTGGCCGATTCATTAATGCAGCT
GGCACGACAGGTTTCCCGACTGGAAAGCGGGCAGTGAGCGCAACGCAAT
TAATGTGAGTTAGCTCACTCATTAGGCACCCCAGGCTTTACACTTTATG
CTTCCGGCTCGTATGTTGTGTGGAATTGTGAGCGGATAACAATTTCACA
CAGGAAACAGCTATGACCATGATTACGCCAAGCTTTGCTCTTAGGAGTT
TCCTAATACATCCCAAACTCAAATATATAAAGCATTTGACTTGTTCTAT
GCCCTAGGGGGCGGGGGGAAGCTAAGCCAGCTTTTTTTAACATTTAAAA
TGTTAATTCCATTTTAAATGCACAGATGTTTTTATTTCATAAGGGTTTC
AATGTGCATGAATGCTGCAATATTCCTGTTACCAAAGCTAGTATAAATA
AAAATAGATAAACGTGGAAATTACTTAGAGTTTCTGTCATTAACGTTTC
CTTCCTCAGTTGACAACATAAATGCGCTGCTGAGCAAGCCAGTTTGCAT
CTGTCAGGATCAATTTCCCATTATGCCAGTCATATTAATTACTAGTCAA
TTAGTTGATTTTTATTTTTGACATATACATGTGAATGAAAGACCCCACC
TGTAGGTTTGGCAAGCTAGCTTAAGTAACGCCATTTTGCAAGGCATGGA
AAAATACATAACTGAGAATAGAAAAGTTCAGATCAAGGTCAGGAACAGA
TGGAACAGCTGAATATGGGCCAAACAGGATATCTGTGGTAAGCAGTTCC
TGCCCCGGCTCAGGGCCAAGAACAGATGGAACAGCTGAATATGGGCCAA
ACAGGATATCTGTGGTAAGCAGTTCCTGCCCCGGCTCAGGGCCAAGAAC
AGATGGTCCCCAGATGCGGTCCAGCCCTCAGCAGTTTCTAGAGAACCAT
CAGATGTTTCCAGGGTGCCCCAAGGACCTGAAATGACCCTGTGCCTTAT
TTGAACTAACCAATCAGTTCGCTTCTCGCTTCTGTTCGCGCGCTTATGC
TCCCCGAGCTCAATAAAAGAGCCCACAACCCCTCACTCGGGGCGCCAGT
CCTCCGATTGACTGAGTCGCCCGGGTACCCGTGTATCCAATAAACCCTC
TTGCAGTTGCATCCGACTTGTGGTCTCGCTGTTCCTTGGGAGGGTCTCC
TCTGAGTGATTGACTACCCGTCAGCGGGGGTCTTTCATTTGGGGGCTCG
TCCGGGATCGGGAGACCCCTGCCCAGGGACCACCGACCCACCACCGGGA
GGTAAGCTGGCCAGCAACTTATCTGTGTCTGTCCGATTGTCTAGTGTCT
ATGACTGATTTTATGCGCCTGCGTCGGTACTAGTTAGCTAACTAGCTCT
GTATCTGGCGGACCCGTGGTGGAACTGACGAGTTCGGAACACCCGGCCG
CAACCCTGGGAGACGTCCCAGGGACTTCGGGGGCCGTTTTTGTGGCCCG
ACCTGAGTCCTAAAATCCCGATCGTTTAGGACTCTTTGGTGCACCCCCC
TTAGAGGAGGGATATGTGGTTCTGGTAGGAGACGAGAACCTAAAACAGT
TCCCGCCTCCGTCTGAATTTTTGCTTTCGGTTTGGGACCGAAGCCGCGC
CGCGCGTCTTGTCTGCTGCAGCATCGTTCTGTGTTGTCTCTGTCTGACT
GTGTTTCTGTATTTGTCTGAAAATATGGGCCCGGGCTAGACTGTTACCA
CTCCCTTAAGTTTGACCTTAGGTCACTGGAAAGATGTCGAGCGGATCGC
TCACAACCAGTCGGTAGATGTCAAGAAGAGACGTTGGGTTACCTTCTGC
TCTGCAGAATGGCCAACCTTTAACGTCGGATGGCCGCGAGACGGCACCT
TTAACCGAGACCTCATCACCCAGGTTAAGATCAAGGTCTTTTCACCTGG
CCCGCATGGACACCCAGACCAGGTCCCCTACATCGTGACCTGGGAAGCC
TTGGCTTTTGACCCCCCTCCCTGGGTCAAGCCCTTTGTACACCCTAAGC
CTCCGCCTCCTCTTCCTCCATCCGCCCCGTCTCTCCCCCTTGAACCTCC
TCGTTCGACCCCGCCTCGATCCTCCCTTTATCCAGCCCTCACTCCTTCT
CTAGGCGCCCCCATATGGCCATATGAGATCTTATATGGGGCACCCCCGC
CCCTTGTAAACTTCCCTGACCCTGACATGACAAGAGTTACTAACAGCCC
CTCTCTCCAAGCTCACTTACAGGCTCTCTACTTAGTCCAGCACGAAGTC
TGGAGACCTCTGGCGGCAGCCTACCAAGAACAACTGGACCGA.

The isolated nucleic acid molecule having the nucleotide sequence of SEQ ID NO:953 encodes a FcRL-5-targeted CAR (designated as 31 FcRL5-targeted BBz CAR) comprising a fully human scFv (encoded by nucleotides 207-998 of SEQ ID NO:953) that comprises a heavy chain variable region comprising amino acids having the sequence set forth in SEQ ID NO:116, a light chain variable region comprising amino acids having the sequence set forth in SEQ ID NO:115, and a linker having an amino acid sequence of SEQ ID NO:307 positioned between the heavy chain variable region and the light chain variable region, a transmembrane domain comprising a CD8 polypeptide having 137 to 207 of SEQ ID NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 270-998 of SEQ ID NO: 953 encodes the human scFv. Nucleotides 1008-1220 of SEQ ID NO: 953 encodes the CD8 polypeptide comprised in the transmembrane domain. Nucleotides 1221-1346 of SEQ ID NO: 953 encodes the 4-1BB polypeptide comprised in the intracellular domain. Nucleotides 1347-1685 of SEQ ID NO: 953 encodes the CD3zeta polypeptide comprised in the intracellular domain. Other portions of SEQ ID NO: 953 are shown in Table 232.

TABLE 232

| | Nucleotide Sequence Positions | Number of |
|---|---|---|
| Portions | of SEQ ID NO: 953 | Nucleotides |
| anti-FcRL5 scFv 31 | 207 . . . 998 | 792 |
| CD8a TM | 1008 . . . 1220 | 213 |
| 4-1BB | 1221 . . . 1346 | 126 |
| CD3zeta | 1347 . . . 1685 | 339 |
| LTR | 1965 . . . 2434 | 470 |
| M13 fwd | 3133 . . . 3149 | 17 |
| AmpR promoter | 3624 . . . 3728 | 105 |
| AmpR | 3729 . . . 4589 | 861 |
| ori | 4760 . . . 5348 | 589 |
| CAP binding site | 5636 . . . 5657 | 22 |
| lac promoter | 5672 . . . 5702 | 31 |
| lac operator | 5710 . . . 5726 | 17 |
| M13 rev | 5734 . . . 5750 | 17 |
| LTR | 6159 . . . 6752 | 594 |
| MMLV Psi | 6815 . . . 7172 | 358 |
| gag (truncated) | 7237 . . . 7653 | 417 |

In certain embodiments, the isolated nucleic acid molecule comprises nucleic acids having the sequence set forth in SEQ ID NO:954 provided below:

[SEQ ID NO: 954]
CCGGTGGTACCTCACCCTTACCGAGTCGGCGACACAGTGTGGGTCCGCCGACACCAGACTAAGAACCTAGAACCTCGCTG
GAAAGGACCTTACACAGTCCTGCTGACCACCCCCACCGCCCTCAAAGTAGACGGCATCGCAGCTTGGATACACGCCGCCC
ACGTGAAGGCTGCCGACCCCGGGGGTGGACCATCCTCTAGACTGCCATGGAAACCGATACACTGCTGCTGTGGGTGCTGC
TGCTGTGGGTGCCAGGATCCACAGGCAATTTTATGCTGACTCAGCCCCACTCTGTGTCGGAGTCTCCGGGGAAGACGGTA
ACCATCTCCTGCACCCGCAGCAGTGGCAGCATTGCCAGCAACTATGTGCAGTGGTACCAGCAGCGCCCGGGCAGTTCCCC
CACCACTGTGATCTATGAGGATAACCAAAGACCCTCTGGGGTCCCTGATCGGTTCTCTGGCTCCATCGACAGCTCCTCCA
ACTCTGCCTCCCTCACCATCTCTGGACTGAAGACTGAGGACGAGGCTGACTACTACTGTCAGTCTTATGATAGCAGCAAT
TGGGTGTTCGGCGGAGGGACCAAGCTGACCGTCCTAGGTTCTAGAGGTGGTGGTGGTAGCGGCGGCGGCGGCTCTGGTGG
TGGTGGATCCCTCGAGATGGCCGAGGTCCAGCTGGTGCAGTCTGGGGCTGAGGTGAAGAAGCCTGGGTCCTCGGTGAAGG
TCTCCTGCAAGGCTTCTGGAGGCACCTTCAGCAGCTATGCTATCAGCTGGGTGCGACAGGCCCCTGGACAAGGGCTTGAG
TGGATGGGAGGGATCATCCCTATCTTTGGTACAGCAAACTACGCACAGAAGTTCCAGGGCAGAGTCACGATTACCGCGGA
CGAATCCACGAGCACAGCCTACATGGAGCTGAGCAGCCTGAGATCTGAGGACACGGCCGTGTATTACTGTGCGCGCTCTA
ACTACTACTACAACGATTACTGGGGTCAAGGTACTCTGGTGACCGTCTCCTCAGCGGCCGCACCCACCACGACGCCAGCG
CCGCGACCACCAACCCCGGCGCCCACGATCGCGTCGCAGCCCCTGTCCCTGCGCCCAGAGGCGTGCCGGCCAGCGGCGGG
GGGCGCAGTGCACACGAGGGGGCTGGACTTCGCCTGTGATATCTACATCTGGGCGCCCCTGGCCGGGACTTGTGGGGTCC
TTCTCCTGTCACTGGTTATCACCCTTTACTGCAACAAACGGGGCAGAAAGAAGCTCCTGTATATATTCAAACAACCATTT
ATGAGACCAGTACAAACTACTCAAGAGGAAGATGGCTGTAGCTGCCGATTTCCAGAAGAAGAAGAAGGAGGATGTGAACT
GAGAGTGAAGTTCAGCAGGAGCGCAGAGCCCCCCGCGTACCAGCAGGGCCAGAACCAGCTCTATAACGAGCTCAATCTAG
GACGAAGAGAGGAGTACGATGTTTTGGACAAGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTACAGTGAGATTGGGATGAAAGGCGAGCG
CCGGAGGGGCAAGGGGCACGATGGCCTTTACCAGGGTCTCAGTACAGCCACCAAGGACACCTACGACGCCCTTCACATGC
AGGCCCTGCCCCCTCGCTAACAGCCACTCGAGGATCCGGATTAGTCCAATTTGTTAAAGACAGGATATCAGTGGTCCAGG
CTCTAGTTTTGACTCAACAATATCACCAGCTGAAGCCTATAGAGTACGAGCCATAGATAAAATAAAAGATTTTATTTAGT
CTCCAGAAAAAGGGGGGAATGAAAGACCCCACCTGTAGGTTTGGCAAGCTAGCTTAAGTAACGCCATTTTGCAAGGCATG
GAAAAATACATAACTGAGAATAGAGAAGTTCAGATCAAGGTCAGGAACAGATGGAACAGCTGAATATGGGCCAAACAGGA
TATCTGTGGTAAGCAGTTCCTGCCCCGGCTCAGGGCCAAGAACAGATGGAACAGCTGAATATGGGCCAAACAGGATATCT
GTGGTAAGCAGTTCCTGCCCCGGCTCAGGGCCAAGAACAGATGGTCCCCAGATGCGGTCCAGCCCTCAGCAGTTTCTAGA
GAACCATCAGATGTTTCCAGGGTGCCCCAAGGACCTGAAATGACCCTGTGCCTTATTTGAACTAACCAATCAGTTCGCTT
CTCGCTTCTGTTCGCGCGCTTCTGCTCCCCGAGCTCAATAAAAGAGCCCACAACCCCTCACTCGGGGCGCCAGTCCTCCG
ATTGACTGAGTCGCCCGGGTACCCGTGTATCCAATAAACCCTCTTGCAGTTGCATCCGACTTGTGGTCTCGCTGTTCCTT
GGGAGGGTCTCCTCTGAGTGATTGACTACCCGTCAGCGGGGGTCTTTCACACATGCAGCATGTATCAAAATTAATTTGGT
TTTTTTTCTTAAGTATTTACATTAAATGGCCATAGTACTTAAAGTTACATTGGCTTCCTTGAAATAAACATGGAGTATTC
AGAATGTGTCATAAATATTTCTAATTTTAAGATAGTATCTCCATTGGCTTTCTACTTTTTCTTTTATTTTTTTTTGTCCT
CTGTCTTCCATTTGTTGTTGTTGTTGTTTGTTTGTTTGTTTGTTGGTTGGTTGGTTAATTTTTTTTTAAAGATCCTACAC
TATAGTTCAAGCTAGACTATTAGCTACTCTGTAACCCAGGGTGACCTTGAAGTCATGGGTAGCCTGCTGTTTTAGCCTTC
CCACATCTAAGATTACAGGTATGAGCTATCATTTTTGGTATATTGATTGATTGATTGATTGATGTGTGTGTGTGTGATTG
TGTTTGTGTGTGTGACTGTGAAAATGTGTGTATGGGTGTGTGTGAATGTGTGTATGTATGTGTGTGTGTGAGTGTGTGTG
TGTGTGTGTGCATGTGTGTGTGTGTGACTGTGTCTATGTGTATGACTGTGTGTGTGTGTGTGTGTGTGTGTGTGTGTGTG
TGTGTGTGTGTGTTGTGAAAAAATATTCTATGGTAGTGAGAGCCAACGCTCCGGCTCAGGTGTCAGGTTGGTTTTTGAGA
CAGAGTCTTTCACTTAGCTTGGAATTCACTGGCCGTCGTTTTACAACGTCGTGACTGGGAAAACCCTGGCGTTACCCAAC
TTAATCGCCTTGCAGCACATCCCCCTTTCGCCAGCTGGCGTAATAGCGAAGAGGCCCGCACCGATCGCCCTTCCCAACAG
TTGCGCAGCCTGAATGGCGAATGGCGCCTGATGCGGTATTTTCTCCTTACGCATCTGTGCGGTATTTCACACCGCATATG
GTGCACTCTCAGTACAATCTGCTCTGATGCCGCATAGTTAAGCCAGCCCCGACACCCGCCAACACCCGCTGACGCGCCCT
GACGGGCTTGTCTGCTCCCGGCATCCGCTTACAGACAAGCTGTGACCGTCTCCGGGAGCTGCATGTGTCAGAGGTTTTCA
CCGTCATCACCGAAACGCGCGATGACGAAAGGGCCTCGTGATACGCCTATTTTTATAGGTTAATGTCATGATAATAATGG
TTTCTTAGACGTCAGGTGGCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACAGCGGTAAGATCCTTGAGAGTTTTCGC
CCCGAAGAACGTTTTCCAATGATGAGCACTTTTAAAGTTCTGCTATGTGGCGCGGTATTATCCCGTATTGACGCCGGGCA
AGAGCAACTCGGTCGCCGCATACACTATTCTCAGAATGACTTGGTTGAGTACTCACCAGTCACAGAAAAGCATCTTACGG
ATGGCATGACAGTAAGAGAATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCTTGATCGTTGGGAACCGGA
GCTGAATGAAGCCATACCAAACGACGAGCGTGACACCACGATGCCTGTAGCAATGGCAACAACGTTGCGCAAACTATTAA
CTGGCGAACTACTTACTCTAGCTTCCCGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGCGTGGGTCTCGCGGTATCATTGCAGC
ACTGGGGCCAGATGGTAAGCCCTCCCGTATCGTAGTTATCTACACGACGGGGAGTCAGGCAACTATGGATGAACGAAATA
GACAGATCGCTGAGATAGGTGCCTCACTGATTAAGCATTGGTAACTGTCAGACCAAGTTTACTCATATATACTTTAGATT
GATTTAAAACTTCATTTTTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGCTACCAACTCTTTTTCCG
AAGGTAACTGGCTTCAGCAGAGCGCAGATACCAAATACTGTCCTTCTAGTGTAGCCGTAGTTAGGCCACCACTTCAAGAA
CTCTGTAGCACCGCCTACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTTCGTGCACACAGCCCAGC
TTGGAGCGAACGACCTACACCGAACTGAGATACCTACAGCGTGAGCATTGAGAAAGCGCCACGCTTCCCGAAGGGAGAAA
GGCGGACAGGTATCCGGTAAGCGGCAGGGTCGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGTTCTTTCCTGCGTTATC
CCCTGATTCTGTGGATAACCGTATTACCGCCTTTGAGTGAGCTGATACCGCTCGCCGCAGCCGAACGACCGAGCGCAGCG
AGTCAGTGAGCGAGGAAGCGGAAGAGCGCCCAATACGCAAACCGCCTCTCCCCGCGCGTTGGCCGATTCATTAATGCAGC
TGGCACGACAGGTTTCCCGACTGGAAAGCGGGCAGTGAGCGCAACGCAATTAATGTGAGTTAGCTCACTCATTAGGCACC
CCAGGCTTTACACTTTATGCTTCCGGCTCGTATGTTGTGTGGAATTGTGAGCGGATAACAATTTCACACAGGAAACAGCT
ATGACCATGATTACGCCAAGCTTTGCTCTTAGGAGTTTCCTAATACATCCCAAACTCAAATATATAAAGCATTTGACTTG
TTCTATGCCCTAGGGGGCGGGGGGAAGCTAAGCCAGCTTTTTTTAACATTTAAAATGTTAATTCCATTTTAAATGCACAG
ATGTTTTTATTTCATAAGGGTTTCAATGTGCATGAATGCTGCAATATTCCTGTTACCAAAGCTAGTATAAATAAAAATAG
ATAAACGTGGAAATTACTTAGAGTTTCTGTCATTAACGTTTCCTTCCTCAGTTGACAACATAAATGCGCTGCTGAGCAAG
CCAGTTTGCATCTGTCAGGATCAATTTCCCATTATGCCAGTCATATTAATTACTAGTCAATTAGTTGATTTTTATTTTTG
ACATATACATGTGAATGAAAGACCCCACCTGTAGGTTTGGCAAGCTAGCTTAAGTAACGCCATTTTGCAAGGCATGGAAA
AATACATAACTGAGAATAGAAAAGTTCAGATCAAGGTCAGGAACAGATGGAACAGCTGAATATGGGCCAAACAGGATATC
TGTGGTAAGCAGTTCCTGCCCCGGCTCAGGGCCAAGAACAGATGGAACAGCTGAATATGGGCCAAACAGGATATCTGTGG
TAAGCAGTTCCTGCCCCGGCTCAGGGCCAAGAACAGATGGTCCCCAGATGCGGTCCAGCCCTCAGCAGTTTCTAGAGAAC
CATCAGATGTTTCCAGGGTGCCCCAAGGACCTGAAATGACCCTGTGCCTTATTTGAACTAACCAATCAGTTCGCTTCTCG
CTTCTGTTCGCGCGCTTATGCTCCCCGAGCTCAATAAAAGAGCCCACAACCCCTCACTCGGGGCGCCAGTCCTCCGATTG
ACTGAGTCGCCCGGGTACCCGTGTATCCAATAAACCCTCTTGCAGTTGCATCCGACTTGTGGTCTCGCTGTTCCTTGGGA
GGGTCTCCTCTGAGTGATTGACTACCCGTCAGCGGGGGTCTTTCATTTGGGGGCTCGTCCGGGATCGGGAGACCCCTGCC
CAGGGACCACCGACCCACCACCGGGAGGTAAGCTGGCCAGCAACTTATCTGTGTCTGTCCGATTGTCTAGTGTCTATGAC
TGATTTTATGCGCCTGCGTCGGTACTAGTTAGCTAACTAGCTCTGTATCTGGCGGACCCGTGGTGGAACTGACGAGTTCG
GAACACCCGGCCGCAACCCTGGGAGACGTCCCAGGGACTTCGGGGGCCGTTTTTGTGGCCCGACCTGAGTCCTAAAATCC
CGATCGTTTAGGACTCTTTGGTGCACCCCCCTTAGAGGAGGGATATGTGGTTCTGGTAGGAGACGAGAACCTAAAACAGT
TCCCGCCTCCGTCTGAATTTTTGCTTTCGGTTTGGGACCGAAGCCGCGCCGCGCGTCTTGTCTGCTGCAGCATCGTTCTG
TGTTGTCTCTGTCTGACTGTGTTTCTGTATTTGTCTGAAAATATGGGCCCGGGCTAGACTGTTACCACTCCCTTAAGTTT
GACCTTAGGTCACTGGAAAGATGTCGAGCGGATCGCTCACAACCAGTCGGTAGATGTCAAGAAGAGACGTTGGGTTACCT
TCTGCTCTGCAGAATGGCCAACCTTTAACGTCGGATGGCCGCGAGACGGCACCTTTAACCGAGACCTCATCACCCAGGTT
AAGATCAAGGTCTTTTCACCTGGCCCGCATGGACACCCAGACCAGGTCCCCTACATCGTGACCTGGGAAGCCTTGGCTTT
TGACCCCCCTCCCTGGGTCAAGCCCTTTGTACACCCTAAGCCTCCGCCTCCTCTTCCTCCATCCGCCCCGTCTCTCCCCC
TTGAACCTCCTCGTTCGACCCCGCCTCGATCCTCCCTTTATCCAGCCCTCACTCCTTCTCTAGGCGCCCCCATATGGCCA
TATGAGATCTTATATGGGGCACCCCCGCCCCTTGTAAACTTCCCTGACCCTGACATGACAAGAGTTACTAACAGCCCCTC
TCTCCAAGCTCACTTACAGGCTCTCTACTTAGTCCAGCACGAAGTCTGGAGACCTCTGGCGGCAGCCTACCAAGAACAAC
TGGACCGA.

The isolated nucleic acid molecule having the nucleotide sequence of SEQ ID NO:954 encodes a FcRL-5-targeted CAR (designated as 39 FcRL5-targeted BBz CAR) comprising a fully human scFv (encoded by nucleotides 207-1013 of SEQ ID NO:954) that comprises a heavy chain variable region comprising amino acids having the sequence set forth in SEQ ID NO:144, a light chain variable region comprising amino acids having the sequence set forth in SEQ ID NO:143, and a linker having an amino acid sequence of SEQ ID NO:307 positioned between the heavy chain variable region and the light chain variable region, a transmembrane domain comprising a CD8 polypeptide having 137 to 207 of SEQ ID NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 207-1013 of SEQ ID NO: 954 encodes the human scFv. Nucleotides 1023-1235 of SEQ ID NO: 954 encodes the CD8 polypeptide comprised in the transmembrane domain. Nucleotides 1236-1361 of SEQ ID NO: 954 encodes the 4-1BB polypeptide comprised in the intracellular domain. Nucleotides 1362-1700 of SEQ ID NO: 954 encodes the CD3zeta polypeptide comprised in the intracellular domain. Other portions of SEQ ID NO: 954 are shown in Table 233.

TABLE 233

| | Nucleotide Sequence | |
|---|---|---|
| | Positions of SEQ ID NO: | |
| Portions | 954 | Number of Nucleotides |
| anti-FcRL5 scFv 39 | 207 . . . 1013 | 807 |
| CD8a TM | 1023 . . . 1235 | 213 |
| 4-1BB | 1236 . . . 1361 | 126 |
| CD3zeta | 1362 . . . 1700 | 339 |
| LTR | 1980 . . . 2449 | 470 |
| M13 fwd | 3148 . . . 3164 | 17 |
| AmpR promoter | 3639 . . . 3743 | 105 |
| AmpR | 3744 . . . 4604 | 861 |
| ori | 4775 . . . 5363 | 589 |
| CAP binding site | 5651 . . . 5672 | 22 |
| lac promoter | 5687 . . . 5717 | 31 |
| lac operator | 5725 . . . 5741 | 17 |
| M13 rev | 5749 . . . 5765 | 17 |
| LTR | 6174 . . . 6767 | 594 |
| MMLV Psi | 6830 . . . 7187 | 358 |
| gag (truncated) | 7252 . . . 7668 | 417 |

In certain embodiments, the isolated nucleic acid molecule comprises nucleic acids having the sequence set forth in SEQ ID NO:955 provided below:

[SEQ ID NO: 955]
CCGGTGGTACCTCACCCTTACCGAGTCGGCGACACAGTGTGGGTCCGCCGACACCAGACTAAGAACCTAGAACCTCGCTG
GAAAGGACCTTACACAGTCCTGCTGACCACCCCCACCGCCCTCAAAGTAGACGGCATCGCAGCTTGGATACACGCCGCCC
ACGTGAAGGCTGCCGACCCCGGGGGTGGACCATCCTCTAGACTGCCATGGAAACCGACACCCTGCTGCTGTGGGTGCTGC
TGCTGTGGGTGCCAGGATCCACAGGACAGTCTGTCGTGACGCAGCCACCCTCAGCGTCTGGGACCCCCGGGCAGAGGGTC
ACCATCTCTTGTTCTGGAAGCAGCTCCAACATCGGAAGTAATTATGTATACTGGTACCAGCAGCTCCCAGGAACGGCCCC
CAAACTCCTCATCTATAGTAATAATCAGCGGCCCTCAGGGGTCCCTGACCGATTCTCTGGCTCCAAGTCTGGCACCTCAG
CCTCCCTGGCCATCAGTGGGCTCCGGTCCGAGGATGAGGCTGATTATTACTGTGCAGCATGGGATGACAGCCTGAGTGGT
TATGTCTTCGGAACTGGGACCAAGCTGACCGTCCTAGGTTCTAGAGGTGGTGGTGGTAGCGGCGGCGGCGGCTCTGGTGG
TGGTGGATCCCTCGAGATGGCCCAGGTGCAGCTACAGCAGTGGGGCGCAGGACTGTTGAAGCCTTCGGAGACCCTGTCCC
TCACCTGCGCTGTCTATGGTGGGTCCTTCAGTGGTTACTACTGGAGCTGGATCCGCCAGCCCCCAGGGAAGGGGCTGGAG
TGGATTGGGGAAATCAATCATAGTGGAAGCACCAACTACAACCCGTCCCTCAAGAGTCGAGTCACCATATCAGTAGACAC
GTCCAAGAACCAGTTCTCCCTGAAGCTGAGCTCTGTGACCGCCGCGGACACGGCCGTGTATTACTGTGCGCGCCTGTACG
AAGGTGGTTACCATGGTTGGGGTTCTTGGCTGTCTTCTGATTCTTGGGGTCAAGGTACTCTGGTGACCGTCTCCTCAGCG
GCCGCACCCACCACGACGCCAGCGCCGCGACCACCAACCCCGGCGCCCACGATCGCGTCGCAGCCCCTGTCCCTGCGCCC
AGAGGCGTGCCGGCCAGCGGCGGGGGGCGCAGTGCACACGAGGGGGCTGGACTTCGCCTGTGATATCTACATCTGGGCGC
CCCTGGCCGGGACTTGTGGGGTCCTTCTCCTGTCACTGGTTATCACCCTTTACTGCAACAAACGGGGCAGAAAGAAGCTC
CTGTATATATTCAAACAACCATTTATGAGACCAGTACAAACTACTCAAGAGGAAGATGGCTGTAGCTGCCGATTTCCAGA
AGAAGAAGAAGGAGGATGTGAACTGAGAGTGAAGTTCAGCAGGAGCGCAGAGCCCCCCGCGTACCAGCAGGGCCAGAACC
AGCTCTATAACGAGCTCAATCTAGGACGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCAGAAAGATAAGATGGCGGAGGCCTACAG
TGAGATTGGGATGAAAGGCGAGCGCCGGAGGGGCAAGGGGCACGATGGCCTTTACCAGGGTCTCAGTACAGCCACCAAGG
ACACCTACGACGCCCTTCACATGCAGGCCCTGCCCCCTCGCTAACAGCCACTCGAGGATCCGGATTAGTCCAATTTGTTA
AAGACAGGATATCAGTGGTCCAGGCTCTAGTTTTGACTCAACAATATCACCAGCTGAAGCCTATAGAGTACGAGCCATAG
ATAAAATAAAAGATTTTATTTAGTCTCCAGAAAAAGGGGGGAATGAAAGACCCCACCTGTAGGTTTGGCAAGCTAGCTTA
AGTAACGCCATTTTGCAAGGCATGGAAAAATACATAACTGAGAATAGAGAAGTTCAGATCAAGGTCAGGAACAGATGGAA
CAGCTGAATATGGGCCAAACAGGATATCTGTGGTAAGCAGTTCCTGCCCCGGCTCAGGGCCAAGAACAGATGGAACAGCT
GAATATGGGCCAAACAGGATATCTGTGGTAAGCAGTTCCTGCCCCGGCTCAGGGCCAAGAACAGATGGTCCCCAGATGCG
GTCCAGCCCTCAGCAGTTTCTAGAGAACCATCAGATGTTTCCAGGGTGCCCCAAGGACCTGAAATGACCCTGTGCCTTAT
TTGAACTAACCAATCAGTTCGCTTCTCGCTTCTGTTCGCGCGCTTCTGCTCCCCGAGCTCAATAAAAGAGCCCACAACCC
CTCACTCGGGGCGCCAGTCCTCCGATTGACTGAGTCGCCCGGGTACCCGTGTATCCAATAAACCCTCTTGCAGTTGCATC
CGACTTGTGGTCTCGCTGTTCCTTGGGAGGGTCTCCTCTGAGTGATTGACTACCCGTCAGCGGGGGTCTTTCACACATGC
AGCATGTATCAAAATTAATTTGGTTTTTTTTCTTAAGTATTTACATTAAATGGCCATAGTACTTAAAGTTACATTGGCTT
CCTTGAAATAAACATGGAGTATTCAGAATGTGTCATAAATATTTCTAATTTTAAGATAGTATCTCCATTGGCTTTCTACT
TTTTCTTTTATTTTTTTTTGTCCTCTGTCTTCCATTTGTTGTTGTTGTTGTTTGTTTGTTTGTTTGTTGGTTGGTTGGTT
AATTTTTTTTTAAAGATCCTACACTATAGTTCAAGCTAGACTATTAGCTACTCTGTAACCCAGGGTGACCTTGAAGTCAT
GGGTAGCCTGCTGTTTTAGCCTTCCCACATCTAAGATTACAGGTATGAGCTATCATTTTTGGTATATTGATTGATTGATT
GATTGATGTGTGTGTGTGTGATTGTGTTTGTGTGTGTGACTGTGAAAATGTGTGTATGGGTGTGTGTGAATGTGTGTATG
TATGTGTGTGTGTGAGTGTGTGTGTGTGTGTGTGCATGTGTGTGTGTGTGACTGTGTCTATGTGTATGACTGTGTGTGTG
TGTGTGTGTGTGTGTGTGTGTGTGTGTGTGTGTGTGTTGTGAAAAAATATTCTATGGTAGTGAGAGCCAACGCTCCGGCT
CAGGTGTCAGGTTGGTTTTTGAGACAGAGTCTTTCACTTAGCTTGGAATTCACTGGCCGTCGTTTTACAACGTCGTGACT
GGGAAAACCCTGGCGTTACCCAACTTAATCGCCTTGCAGCACATCCCCCTTTCGCCAGCTGGCGTAATAGCGAAGAGGCC
CGCACCGATCGCCCTTCCCAACAGTTGCGCAGCCTGAATGGCGAATGGCGCCTGATGCGGTATTTTCTCCTTACGCATCT
GTGCGGTATTTCACACCGCATATGGTGCACTCTCAGTACAATCTGCTCTGATGCCGCATAGTTAAGCCAGCCCCGACACC
CGCCAACACCCGCTGACGCGCCCTGACGGGCTTGTCTGCTCCCGGCATCCGCTTACAGACAAGCTGTGACCGTCTCCGGG
AGCTGCATGTGTCAGAGGTTTTCACCGTCATCACCGAAACGCGCGATGACGAAAGGGCCTCGTGATACGCCTATTTTTAT
AGGTTAATGTCATGATAATAATGGTTTCTTAGACGTCAGGTGGCACTTTTCGGGGAAATGTGCGCGGAACCCCTATTTGT
TTATTTTTCTAAATACATTCAAATATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTTTGCCTTCCTGTTTTTGCTCACC
CAGAAACGCTGGTGAAAGTAAAAGATGCTGAAGATCAGTTGGGTGCACGAGTGGGTTACATCGAACTGGATCTCAACAGC
GGTAAGATCCTTGAGAGTTTTCGCCCCGAAGAACGTTTTCCAATGATGAGCACTTTTAAAGTTCTGCTATGTGGCGCGGT
ATTATCCCGTATTGACGCCGGGCAAGAGCAACTCGGTCGCCGCATACACTATTCTCAGAATGACTTGGTTGAGTACTCAC
CAGTCACAGAAAAGCATCTTACGGATGGCATGACAGTAAGAGAATTATGCAGTGCTGCCATAACCATGAGTGATAACACT
GCGGCCAACTTACTTCTGACAACGATCGGAGGACCGAAGGAGCTAACCGCTTTTTTGCACAACATGGGGGATCATGTAAC
TCGCCTTGATCGTTGGGAACCGGAGCTGAATGAAGCCATACCAAACGACGAGCGTGACACCACGATGCCTGTAGCAATGG
CAACAACGTTGCGCAAACTATTAACTGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTGCTGATAAATCTGGAGCCGGTGAGCG
TGGGTCTCGCGGTATCATTGCAGCACTGGGGCCAGATGGTAAGCCCTCCCGTATCGTAGTTATCTACACGACGGGGAGTC
AGGCAACTATGGATGAACGAAATAGACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGATCTAGGTGAAGATCCTTTTTGATAA
TCTCATGACCAAAATCCCTTAACGTGAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACCAAATACTGTCCTTCTAGTGTAGCC
GTAGTTAGGCCACCACTTCAAGAACTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTACAGCGTGAGCATTGAGAAAG
CGCCACGCTTCCCGAAGGGAGAAAGGCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCTTTGAGTGAGCTGATACCGCTCGCC
GCAGCCGAACGACCGAGCGCAGCGAGTCAGTGAGCGAGGAAGCGGAAGAGCGCCCAATACGCAAACCGCCTCTCCCCGCG
CGTTGGCCGATTCATTAATGCAGCTGGCACGACAGGTTTCCCGACTGGAAAGCGGGCAGTGAGCGCAACGCAATTAATGT
GAGTTAGCTCACTCATTAGGCACCCCAGGCTTTACACTTTATGCTTCCGGCTCGTATGTTGTGTGGAATTGTGAGCGGAT
AACAATTTCACACAGGAAACAGCTATGACCATGATTACGCCAAGCTTTGCTCTTAGGAGTTTCCTAATACATCCCAAACT
CAAATATATAAAGCATTTGACTTGTTCTATGCCCTAGGGGGCGGGGGGAAGCTAAGCCAGCTTTTTTTAACATTTAAAAT
GTTAATTCCATTTTAAATGCACAGATGTTTTTATTTCATAAGGGTTTCAATGTGCATGAATGCTGCAATATTCCTGTTAC
CAAAGCTAGTATAAATAAAAATAGATAAACGTGGAAATTACTTAGAGTTTCTGTCATTAACGTTTCCTTCCTCAGTTGAC
AACATAAATGCGCTGCTGAGCAAGCCAGTTTGCATCTGTCAGGATCAATTTCCCATTATGCCAGTCATATTAATTACTAG
TCAATTAGTTGATTTTTATTTTTGACATATACATGTGAATGAAAGACCCCACCTGTAGGTTTGGCAAGCTAGCTTAAGTA
ACGCCATTTTGCAAGGCATGGAAAAATACATAACTGAGAATAGAAAAGTTCAGATCAAGGTCAGGAACAGATGGAACAGC
TGAATATGGGCCAAACAGGATATCTGTGGTAAGCAGTTCCTGCCCCGGCTCAGGGCCAAGAACAGATGGAACAGCTGAAT
ATGGGCCAAACAGGATATCTGTGGTAAGCAGTTCCTGCCCCGGCTCAGGGCCAAGAACAGATGGTCCCCAGATGCGGTCC
AGCCCTCAGCAGTTTCTAGAGAACCATCAGATGTTTCCAGGGTGCCCCAAGGACCTGAAATGACCCTGTGCCTTATTTGA
ACTAACCAATCAGTTCGCTTCTCGCTTCTGTTCGCGCGCTTATGCTCCCCGAGCTCAATAAAAGAGCCCACAACCCCTCA
CTCGGGGCGCCAGTCCTCCGATTGACTGAGTCGCCCGGGTACCCGTGTATCCAATAAACCCTCTTGCAGTTGCATCCGAC
TTGTGGTCTCGCTGTTCCTTGGGAGGGTCTCCTCTGAGTGATTGACTACCCGTCAGCGGGGGTCTTTCATTTGGGGGCTC
GTCCGGGATCGGGAGACCCCTGCCCAGGGACCACCGACCCACCACCGGGAGGTAAGCTGGCCAGCAACTTATCTGTGTCT
GTCCGATTGTCTAGTGTCTATGACTGATTTTATGCGCCTGCGTCGGTACTAGTTAGCTAACTAGCTCTGTATCTGGCGGA
CCCGTGGTGGAACTGACGAGTTCGGAACACCCGGCCGCAACCCTGGGAGACGTCCCAGGGACTTCGGGGGCCGTTTTTGT
GGCCCGACCTGAGTCCTAAAATCCCGATCGTTTAGGACTCTTTGGTGCACCCCCCTTAGAGGAGGGATATGTGGTTCTGG
TAGGAGACGAGAACCTAAAACAGTTCCCGCCTCCGTCTGAATTTTTGCTTTCGGTTTGGGACCGAAGCCGCGCCGCGCGT
CTTGTCTGCTGCAGCATCGTTCTGTGTTGTCTCTGTCTGACTGTGTTTCTGTATTTGTCTGAAAATATGGGCCCGGGCTA
GACTGTTACCACTCCCTTAAGTTTGACCTTAGGTCACTGGAAAGATGTCGAGCGGATCGCTCACAACCAGTCGGTAGATG
TCAAGAAGAGACGTTGGGTTACCTTCTGCTCTGCAGAATGGCCAACCTTTAACGTCGGATGGCCGCGAGACGGCACCTTT
AACCGAGACCTCATCACCCAGGTTAAGATCAAGGTCTTTTCACCTGGCCCGCATGGACACCCAGACCAGGTCCCCTACAT
CGTGACCTGGGAAGCCTTGGCTTTTGACCCCCCTCCCTGGGTCAAGCCCTTTGTACACCCTAAGCCTCCGCCTCCTCTTC
CTCCATCCGCCCCGTCTCTCCCCCTTGAACCTCCTCGTTCGACCCCGCCTCGATCCTCCCTTTATCCAGCCCTCACTCCT
TCTCTAGGCGCCCCCATATGGCCATATGAGATCTTATATGGGGCACCCCCGCCCCTTGTAAACTTCCCTGACCCTGACAT
GACAAGAGTTACTAACAGCCCCTCTCTCCAAGCTCACTTACAGGCTCTCTACTTAGTCCAGCACGAAGTCTGGAGACCTC
TGGCGGCAGCCTACCAAGAACAACTGGACCGA.

The isolated nucleic acid molecule having the nucleotide sequence of SEQ ID NO:955 encodes a FcRL-5-targeted CAR (designated as 69 FcRL5-targeted BBz CAR) comprising a fully human scFv (encoded by nucleotides 207-1037 of SEQ ID NO:955) that comprises a heavy chain variable region comprising amino acids having the sequence set forth in SEQ ID NO: 172, a light chain variable region comprising amino acids having the sequence set forth in SEQ ID NO:171, and a linker having an amino acid sequence of SEQ ID NO:307 positioned between the heavy chain variable region and the light chain variable region, a transmembrane domain comprising a CD8 polypeptide having 137 to 207 of SEQ TD NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 207-1037 of SEQ ID NO: 955 encodes the human scFv. Nucleotides 1047-1259 of SEQ ID NO: 955 encodes the CD8 polypeptide comprised in the transmembrane domain. Nucleotides 1260-1385 of SEQ ID NO: 955 encodes the 4-1BB polypeptide comprised in the intracellular domain. Nucleotides 1386-1724 of SEQ ID NO: 955 encodes the CD3zeta polypeptide comprised in the intracellular domain. Other portions of SEQ ID NO: 955 are shown in Table 234.

TABLE 234

| | Nucleotide Sequence | |
|---|---|---|
| | Positions of SEQ ID NO: | |
| Portions | 955 | Number of Nucleotides |
| anti-FcRL5 scFv 69 | 207 . . . 1037 | 831 |
| CD8a TM | 1047 . . . 1259 | 213 |
| 4-1BB | 1260 . . . 1385 | 126 |
| CD3zeta | 1386 . . . 1724 | 339 |
| LTR | 2004 . . . 2473 | 470 |
| M13 fwd | 3172 . . . 3188 | 17 |
| AmpR promoter | 3663 . . . 3767 | 105 |
| AmpR | 3768 . . . 4628 | 861 |
| ori | 4799 . . . 5387 | 589 |
| CAP binding site | 5675 . . . 5696 | 22 |
| lac promoter | 5711 . . . 5741 | 31 |
| lac operator | 5749 . . . 5765 | 17 |
| M13 rev | 5773 . . . 5789 | 17 |
| LTR | 6198 . . . 6791 | 594 |
| MMLV Psi | 6854 . . . 7211 | 358 |
| gag (truncated) | 7276 . . . 7692 | 417 |

In certain embodiments, the isolated nucleic acid molecule comprises nucleic acids having the sequence set forth in SEQ ID NO:956 provided below:

[SEQ ID NO: 956]
CCGGTGGTACCTCACCCTTACCGAGTCGGCGACACAGTGTGGGTCCGCCGACACCAGACTAAGAACCTAGAACCTCGCTG
GAAAGGACCTTACACAGTCCTGCTGACCACCCCCACCGCCCTCAAAGTAGACGGCATCGCAGCTTGGATACACGCCGCCC
ACGTGAAGGCTGCCGACCCCGGGGGTGGACCATCCTCTAGACTGCCATGGAAACCGACACCCTGCTGCTGTGGGTGCTGC
TGCTGTGGGTGCCAGGATCCACAGGAAATTTTATGCTGACTCAGCCCCACTCTGTGTCGGAGTCTCCGGGGAAGACGGTA
ACCATCTCCTGCACCCGCAGCAGTGGCAGCATTGCCAGCAACTATGTGCAGTGGTACCAGCAGCGCCCGGGCAGTGCCCC
CACCACTGTGATCTATGAGGATAACCAAAGACCCTCTGGGGTCCCTGATCGGTTCTCTGGCTCCATCGACAGCTCCTCCA
ACTCTGCCTCCCTCACCATCTCTGGACTGAAGACTGAGGACGAGGCTGACTACTACTGTCAGTCTTATGATAGCAGCAAT
GTGGTATTCGGCGGAGGGACCAAGGTCACCGTCCTAGGTTCTAGAGGTGGTGGTGGTAGCGGCGGCGGCGGCTCTGGTGG
TGGTGGATCCCTCGAGATGGCCGAGGTGCAGCTGGTGGAGTCTGGGGGAGGCTTGGTACAGCCTGGAGGGTCCCTGAGAC
TCTCCTGTGCAGCCTCTGGATTCACCTTCAGTAGTTATGAAATGAACTGGGTCCGCCAGGCTCCAGGGAAGGGGCTGGAG
TGGGTTTCATACATTAGTAGTAGTGGTAGTACCATATACTACGCAGACTCTGTGAAGGGCCGATTCACCATCTCCAGAGA
CAACGCCAAGAACTCACTGTATCTGCAAATGAACAGCCTGAGAGCCGAGGACACGGCTGTTTATTACTGTGCACGCTGGG
ACTACGGTATGGACGTCTGGGGCCAAGGGACCACGGTCACCGTCTCCTCAGCGGCCGCACCCACCACGACGCCAGCGCCG
CGACCACCAACCCCGGCGCCCACGATCGCGTCGCAGCCCCTGTCCCTGCGCCCAGAGGCGTGCCGGCCAGCGGCGGGGGG
CGCAGTGCACACGAGGGGGCTGGACTTCGCCTGTGATATCTACATCTGGGCGCCCCTGGCCGGGACTTGTGGGGTCCTTC
TCCTGTCACTGGTTATCACCCTTTACTGCAACAAACGGGGCAGAAAGAAGCTCCTGTATATATTCAAACAACCATTTATG
AGACCAGTACAAACTACTCAAGAGGAAGATGGCTGTAGCTGCCGATTTCCAGAAGAAGAAGAAGGAGGATGTGAACTGAG
AGTGAAGTTCAGCAGGAGCGCAGAGCCCCCCGCGTACCAGCAGGGCCAGAACCAGCTCTATAACGAGCTCAATCTAGGAC
GAAGAGAGGAGTACGATGTTTTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAGGACACCTACGACGCCCTTCACATGCAGG
CCCTGCCCCCTCGCTAACAGCCACTCGAGGATCCGGATTAGTCCAATTTGTTAAAGACAGGATATCAGTGGTCCAGGCTC
TAGTTTTGACTCAACAATATCACCAGCTGAAGCCTATAGAGTACGAGCCATAGATAAAATAAAAGATTTTATTTAGTCTC
CAGAAAAAGGGGGGAATGAAAGACCCCACCTGTAGGTTTGGCAAGCTAGCTTAAGTAACGCCATTTTGCAAGGCATGGAA
AAATACATAACTGAGAATAGAGAAGTTCAGATCAAGGTCAGGAACAGATGGAACAGCTGAATATGGGCCAAACAGGATAT
CTGTGGTAAGCAGTTCCTGCCCCGGCTCAGGGCCAAGAACAGATGGAACAGCTGAATATGGGCCAAACAGGATATCTGTG
GTAAGCAGTTCCTGCCCCGGCTCAGGGCCAAGAACAGATGGTCCCCAGATGCGGTCCAGCCCTCAGCAGTTTCTAGAGAA
CCATCAGATGTTTCCAGGGTGCCCCAAGGACCTGAAATGACCCTGTGCCTTATTTGAACTAACCAATCAGTTCGCTTCTC
GCTTCTGTTCGCGCGCTTCTGCTCCCCGAGCTCAATAAAAGAGCCCACAACCCCTCACTCGGGGCGCCAGTCCTCCGATT
GACTGAGTCGCCCGGGTACCCGTGTATCCAATAAACCCTCTTGCAGTTGCATCCGACTTGTGGTCTCGCTGTTCCTTGGG
AGGGTCTCCTCTGAGTGATTGACTACCCGTCAGCGGGGGTCTTTCACACATGCAGCATGTATCAAAATTAATTTGGTTTT
TTTTCTTAAGTATTTACATTAAATGGCCATAGTACTTAAAGTTACATTGGCTTCCTTGAAATAAACATGGAGTATTCAGA
ATGTGTCATAAATATTTCTAATTTTAAGATAGTATCTCCATTGGCTTTCTACTTTTTCTTTTATTTTTTTTTGTCCTCTG
TCTTCCATTTGTTGTTGTTGTTGTTTGTTTGTTTGTTTGTTGGTTGGTTGGTTAATTTTTTTTTAAAGATCCTACACTAT
AGTTCAAGCTAGACTATTAGCTACTCTGTAACCCAGGGTGACCTTGAAGTCATGGGTAGCCTGCTGTTTTAGCCTTCCCA
CATCTAAGATTACAGGTATGAGCTATCATTTTTGGTATATTGATTGATTGATTGATTGATGTGTGTGTGTGTGATTGTGT
TTGTGTGTGTGACTGTGAAAATGTGTGTATGGGTGTGTGTGAATGTGTGTATGTATGTGTGTGTGTGAGTGTGTGTGTGT
GTGTGTGCATGTGTGTGTGTGTGACTGTGTCTATGTGTATGACTGTGTGTGTGTGTGTGTGTGTGTGTGTGTGTGTGTGT
GTGTGTGTGTTGTGAAAAAATATTCTATGGTAGTGAGAGCCAACGCTCCGGCTCAGGTGTCAGGTTGGTTTTTGAGACAG
AGTCTTTCACTTAGCTTGGAATTCACTGGCCGTCGTTTTACAACGTCGTGACTGGGAAAACCCTGGCGTTACCCAACTTA
ATCGCCTTGCAGCACATCCCCCTTTCGCCAGCTGGCGTAATAGCGAAGAGGCCCGCACCGATCGCCCTTCCCAACAGTTG
CGCAGCCTGAATGGCGAATGGCGCCTGATGCGGTATTTTCTCCTTACGCATCTGTGCGGTATTTCACACCGCATATGGTG
CACTCTCAGTACAATCTGCTCTGATGCCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAGCTGCATGTGTCAGAGGTTTTCACCG
TCATCACCGAAACGCGCGATGACGAAAGGGCCTCGTGATACGCCTATTTTTATAGGTTAATGTCATGATAATAATGGTTT
CTTAGACGTCAGGTGGCACTTTTCGGGGAAATGTGCGCGGAACCCCTATTTGTTTATTTTTCTAAATACATTCAAATATG
TATCCGCTCATGAGACAATAACCCTGATAAATGCTTCAATAATATTGAAAAAGGAAGAGTATGAGTATTCAACATTTCCG
TGTCGCCCTTATTCCCTTTTTTGCGGCATTTTGCCTTCCTGTTTTTGCTCACCCAGAAACGCTGGTGAAAGTAAAAGATG
CTGAAGATCAGTTGGGTGCACGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCCCGTATTGACGCCGGGCAAGA
GCAACTCGGTCGCCGCATACACTATTCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTGCGGCCAACTTACTTCTGACAACGATC
GGAGGACCGAAGGAGCTAACCGCTTTTTTGCACAACATGGGGGATCATGTAACTCGCCTTGATCGTTGGGAACCGGAGCT
GAATGAAGCCATACCAAACGACGAGCGTGACACCACGATGCCTGTAGCAATGGCAACAACGTTGCGCAAACTATTAACTG
GCGAACTACTTACTCTAGCTTCCCGGCAACAATTAATAGACTGGATGGAGGCGGATAAAGTTGCAGGACCACTTCTGCGC
TCGGCCCTTCCGGCTGGCTGGTTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAACTATGGATGAACGAAATAGAC
AGATCGCTGAGATAGGTGCCTCACTGATTAAGCATTGGTAACTGTCAGACCAAGTTTACTCATATATACTTTAGATTGAT
TTAAAACTTCATTTTTAATTTAAAAGGATCTAGGTGAAGATCCTTTTTGATAATCTCATGACCAAAATCCCTTAACGTGA
GTTTTCGTTCCACTGAGCGTCAGACCCCGTAGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTAGCCGTAGTTAGGCCACCACTTCAAGAACTC
TGTAGCACCGCCTACATACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCATTGAGAAAGCGCCACGCTTCCCGAAGGGAGAAAGGC
GGACAGGTATCCGGTAAGCGGCAGGGTCGGAACAGGAGAGCGCACGAGGGAGCTTCCAGGGGGAAACGCCTGGTATCTTT
ATAGTCCTGTCGGGTTTCGCCACCTCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCACATGTTCTTTCCTGCGTTATCCCC
TGATTCTGTGGATAACCGTATTACCGCCTTTGAGTGAGCTGATACCGCTCGCCGCAGCCGAACGACCGAGCGCAGCGAGT
CAGTGAGCGAGGAAGCGGAAGAGCGCCCAATACGCAAACCGCCTCTCCCCGCGCGTTGGCCGATTCATTAATGCAGCTGG
CACGACAGGTTTCCCGACTGGAAAGCGGGCAGTGAGCGCAACGCAATTAATGTGAGTTAGCTCACTCATTAGGCACCCCA
GGCTTTACACTTTATGCTTCCGGCTCGTATGTTGTGTGGAATTGTGAGCGGATAACAATTTCACACAGGAAACAGCTATG
ACCATGATTACGCCAAGCTTTGCTCTTAGGAGTTTCCTAATACATCCCAAACTCAAATATATAAAGCATTTGACTTGTTC
TATGCCCTAGGGGGCGGGGGGAAGCTAAGCCAGCTTTTTTTAACATTTAAAATGTTAATTCCATTTTAAATGCACAGATG
TTTTTATTTCATAAGGGTTTCAATGTGCATGAATGCTGCAATATTCCTGTTACCAAAGCTAGTATAAATAAAAATAGATA
AACGTGGAAATTACTTAGAGTTTCTGTCATTAACGTTTCCTTCCTCAGTTGACAACATAAATGCGCTGCTGAGCAAGCCA
GTTTGCATCTGTCAGGATCAATTTCCCATTATGCCAGTCATATTAATTACTAGTCAATTAGTTGATTTTTATTTTTGACA
TATACATGTGAATGAAAGACCCCACCTGTAGGTTTGGCAAGCTAGCTTAAGTAACGCCATTTTGCAAGGCATGGAAAAAT
ACATAACTGAGAATAGAAAAGTTCAGATCAAGGTCAGGAACAGATGGAACAGCTGAATATGGGCCAAACAGGATATCTGT
GGTAAGCAGTTCCTGCCCCGGCTCAGGGCCAAGAACAGATGGAACAGCTGAATATGGGCCAAACAGGATATCTGTGGTAA
GCAGTTCCTGCCCCGGCTCAGGGCCAAGAACAGATGGTCCCCAGATGCGGTCCAGCCCTCAGCAGTTTCTAGAGAACCAT
CAGATGTTTCCAGGGTGCCCCAAGGACCTGAAATGACCCTGTGCCTTATTTGAACTAACCAATCAGTTCGCTTCTCGCTT
CTGTTCGCGCGCTTATGCTCCCCGAGCTCAATAAAAGAGCCCACAACCCCTCACTCGGGGCGCCAGTCCTCCGATTGACT
GAGTCGCCCGGGTACCCGTGTATCCAATAAACCCTCTTGCAGTTGCATCCGACTTGTGGTCTCGCTGTTCCTTGGGAGGG
TCTCCTCTGAGTGATTGACTACCCGTCAGCGGGGGTCTTTCATTTGGGGGCTCGTCCGGGATCGGGAGACCCCTGCCCAG
GGACCACCGACCCACCACCGGGAGGTAAGCTGGCCAGCAACTTATCTGTGTCTGTCCGATTGTCTAGTGTCTATGACTGA
TTTTATGCGCCTGCGTCGGTACTAGTTAGCTAACTAGCTCTGTATCTGGCGGACCCGTGGTGGAACTGACGAGTTCGGAA
CACCCGGCCGCAACCCTGGGAGACGTCCCAGGGACTTCGGGGGCCGTTTTTGTGGCCCGACCTGAGTCCTAAAATCCCGA
TCGTTTAGGACTCTTTGGTGCACCCCCCTTAGAGGAGGGATATGTGGTTCTGGTAGGAGACGAGAACCTAAAACAGTTCC
CGCCTCCGTCTGAATTTTTGCTTTCGGTTTGGGACCGAAGCCGCGCCGCGCGTCTTGTCTGCTGCAGCATCGTTCTGTGT
TGTCTCTGTCTGACTGTGTTTCTGTATTTGTCTGAAAATATGGGCCCGGGCTAGACTGTTACCACTCCCTTAAGTTTGAC
CTTAGGTCACTGGAAAGATGTCGAGCGGATCGCTCACAACCAGTCGGTAGATGTCAAGAAGAGACGTTGGGTTACCTTCT
GCTCTGCAGAATGGCCAACCTTTAACGTCGGATGGCCGCGAGACGGCACCTTTAACCGAGACCTCATCACCCAGGTTAAG
ATCAAGGTCTTTTCACCTGGCCCGCATGGACACCCAGACCAGGTCCCCTACATCGTGACCTGGGAAGCCTTGGCTTTTGA
CCCCCCTCCCTGGGTCAAGCCCTTTGTACACCCTAAGCCTCCGCCTCCTCTTCCTCCATCCGCCCCGTCTCTCCCCCTTG
AACCTCCTCGTTCGACCCCGCCTCGATCCTCCCTTTATCCAGCCCTCACTCCTTCTCTAGGCGCCCCCATATGGCCATAT
GAGATCTTATATGGGGCACCCCCGCCCCTTGTAAACTTCCCTGACCCTGACATGACAAGAGTTACTAACAGCCCCTCTCT
CCAAGCTCACTTACAGGCTCTCTACTTAGTCCAGCACGAAGTCTGGAGACCTCTGGCGGCAGCCTACCAAGAACAACTGG
ACCGA.

The isolated nucleic acid molecule having the nucleotide sequence of SEQ ID NO:956 encodes a FcRL-5-targeted CAR (designated as 104 FcRL5-targeted BBz CAR) comprising a fully human scFv (encoded by nucleotides 207-1010) that comprises a heavy chain variable region comprising amino acids having the sequence set forth in SEQ ID NO:216, a light chain variable region comprising amino acids having the sequence set forth in SEQ ID NO:215, and a linker having an amino acid sequence of SEQ ID NO:307 positioned between the heavy chain variable region and the light chain variable region, a transmembrane domain comprising a CD8 polypeptide having 137 to 207 of SEQ ID NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 207-1010 of SEQ ID NO: 956 encodes the human scFv. Nucleotides 1020-1232 of SEQ ID NO: 956 encodes the CD8 polypeptide comprised in the transmembrane domain. Nucleotides 1233-1358 of SEQ ID NO: 956 encodes the 4-1BB polypeptide comprised in the intracellular domain. Nucleotides 1359-1697 of SEQ ID NO: 956 encodes the CD3zeta polypeptide comprised in the intracellular domain. Other portions of SEQ ID NO: 956 are shown in Table 235.

TABLE 235

| | Nucleotide Sequence | |
|---|---|---|
| | Positions of SEQ ID NO: | |
| Portions | 956 | Number of Nucleotides |
| antiFcRL5 scFv 104 | 207 . . . 1010 | 804 |
| CD8a TM | 1020 . . . 1232 | 213 |
| 4-1BB | 1233 . . . 1358 | 126 |
| CD3zeta | 1359 . . . 1697 | 339 |
| LTR | 1977 . . . 2446 | 470 |
| M13 fwd | 3145 . . . 3161 | 17 |
| AmpR promoter | 3636 . . . 3740 | 105 |
| AmpR | 3741 . . . 4601 | 861 |
| ori | 4772 . . . 5360 | 589 |
| CAP binding site | 5648 . . . 5669 | 22 |
| lac promoter | 5684 . . . 5714 | 31 |
| lac operator | 5722 . . . 5738 | 17 |
| M13 rev | 5746 . . . 5762 | 17 |
| LTR | 6171 . . . 6764 | 594 |
| MMLV Psi | 6827 . . . 7184 | 358 |
| gag (truncated) | 7249 . . . 7665 | 417 |

In certain embodiments, the isolated nucleic acid molecule comprises nucleic acids having the sequence set forth in SEQ ID NO:957 provided below:

[SEQ ID NO: 957]
CCGGTGGTACCTCACCCTTACCGAGTCGGCGACACAGTGTGGGTCCGCCGACACCAGACTAAGAACCTAGAACCTCGCTG
GAAAGGACCTTACACAGTCCTGCTGACCACCCCCACCGCCCTCAAAGTAGACGGCATCGCAGCTTGGATACACGCCGCCC
ACGTGAAGGCTGCCGACCCCGGGGGTGGACCATCCTCTAGACTGCCATGGAAACCGATACACTGCTGCTGTGGGTGCTGC
TGCTGTGGGTGCCAGGATCCACAGGCTCCTATGTGCTGACTCAGCCACCCTCAGTGTCCGTGTCCCCAGGACAGACAGCC
AGCATCACCTGCTCTGGAGATAGATTGACGAATAAATATGTTTCCTGGTATCAACAGAAGCCAGGCCAGTCCCCTGTGTT
GGTCATCTATGAGGATGCCAAGCGGCCCTCAGGGATCCCTGCGCGATTCTCTGGCTCCAACTCTGGGAACACAGCCACTC
TGACCATCAGCGGGACCCAGGCTATGGATGAGTCTGAATATTACTGTCAGGCGTGGGACAGCAGTGTGGTGGTTTTTGGC
GGAGGGACCAAGCTGACCGTCCTAGGTTCTAGAGGTGGTGGTGGTAGCGGCGGCGGCGGCTCTGGTGGTGGTGGATCCCT
CGAGATGGCCGAGGTGCAGCTGGTGGAGTCTGGGGGAGGCTTGGTACAGCCTGGCAGGTCCCTGAGACTCTCCTGTGCAG
CCTCTGGATTTACCTTTGATGATTATGCCATGCACTGGGTCCGGCAAGCTCCAGGGAAGGGCCTGGAGTGGGTCTCAGGT
ATTAGTTGGAATAGTGGTAGTATAGGCTATGCGGACTCTGTGAAGGGCCGATTCACCATCTCCAGAGACAACGCCAAGAA
CTCCCTGTATCTGCAAATGAACAGTCTGAGAGATGAGGACACGGCCTTGTATTACTGTGCAAAAGACCGAGGGGGGGGAG
TTATCGTTAAGGATGCTTTTGATATCTGGGGCCAAGGGACAATGGTCACCGTCTCTTCAGCGGCCGCACCCACCACGACG
CCAGCGCCGCGACCACCAACCCCGGCGCCCACGATCGCGTCGCAGCCCCTGTCCCTGCGCCCAGAGGCGTGCCGGCCAGC
GGCGGGGGGCGCAGTGCACACGAGGGGGCTGGACTTCGCCTGTGATATCTACATCTGGGCGCCCCTGGCCGGGACTTGTG
GGGTCCTTCTCCTGTCACTGGTTATCACCCTTTACTGCAACAAACGGGGCAGAAAGAAGCTCCTGTATATATTCAAACAA
CCATTTATGAGACCAGTACAAACTACTCAAGAGGAAGATGGCTGTAGCTGCCGATTTCCAGAAGAAGAAGAAGGAGGATG
TGAACTGAGAGTGAAGTTCAGCAGGAGCGCAGAGCCCCCCGCGTACCAGCAGGGCCAGAACCAGCTCTATAACGAGCTCA
ATCTAGGACGAAGAGAGGAGTACGATGTTTTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCGGAGGCCTACAGTGAGATTGGGATGAAAGG
CGAGCGCCGGAGGGGCAAGGGGCACGATGGCCTTTACCAGGGTCTCAGTACAGCCACCAAGGACACCTACGACGCCCTTC
ACATGCAGGCCCTGCCCCCTCGCTAACAGCCACTCGAGGATCCGGATTAGTCCAATTTGTTAAAGACAGGATATCAGTGG
TCCAGGCTCTAGTTTTGACTCAACAATATCACCAGCTGAAGCCTATAGAGTACGAGCCATAGATAAAATAAAAGATTTTA
TTTAGTCTCCAGAAAAAGGGGGGAATGAAAGACCCCACCTGTAGGTTTGGCAAGCTAGCTTAAGTAACGCCATTTTGCAA
GGCATGGAAAAATACATAACTGAGAATAGAGAAGTTCAGATCAAGGTCAGGAACAGATGGAACAGCTGAATATGGGCCAA
ACAGGATATCTGTGGTAAGCAGTTCCTGCCCCGGCTCAGGGCCAAGAACAGATGGAACAGCTGAATATGGGCCAAACAGG
ATATCTGTGGTAAGCAGTTCCTGCCCCGGCTCAGGGCCAAGAACAGATGGTCCCCAGATGCGGTCCAGCCCTCAGCAGTT
TCTAGAGAACCATCAGATGTTTCCAGGGTGCCCCAAGGACCTGAAATGACCCTGTGCCTTATTTGAACTAACCAATCAGT
TCGCTTCTCGCTTCTGTTCGCGCGCTTCTGCTCCCCGAGCTCAATAAAAGAGCCCACAACCCCTCACTCGGGGCGCCAGT
CCTCCGATTGACTGAGTCGCCCGGGTACCCGTGTATCCAATAAACCCTCTTGCAGTTGCATCCGACTTGTGGTCTCGCTG
TTCCTTGGGAGGGTCTCCTCTGAGTGATTGACTACCCGTCAGCGGGGGTCTTTCACACATGCAGCATGTATCAAAATTAA
TTTGGTTTTTTTTCTTAAGTATTTACATTAAATGGCCATAGTACTTAAAGTTACATTGGCTTCCTTGAAATAAACATGGA
GTATTCAGAATGTGTCATAAATATTTCTAATTTTAAGATAGTATCTCCATTGGCTTTCTACTTTTTCTTTTATTTTTTTT
TGTCCTCTGTCTTCCATTTGTTGTTGTTGTTGTTTGTTTGTTTGTTTGTTGGTTGGTTGGTTAATTTTTTTTTAAAGATC
CTACACTATAGTTCAAGCTAGACTATTAGCTACTCTGTAACCCAGGGTGACCTTGAAGTCATGGGTAGCCTGCTGTTTTA
GCCTTCCCACATCTAAGATTACAGGTATGAGCTATCATTTTTGGTATATTGATTGATTGATTGATTGATGTGTGTGTGTG
TGATTGTGTTTGTGTGTGTGACTGTGAAAATGTGTGTATGGGTGTGTGTGAATGTGTGTATGTATGTGTGTGTGTGAGTG
TGTGTGTGTGTGTGTGCATGTGTGTGTGTGTGACTGTGTCTATGTGTATGACTGTGTGTGTGTGTGTGTGTGTGTGTGTG
TGTGTGTGTGTGTGTGTGTTGTGAAAAAATATTCTATGGTAGTGAGAGCCAACGCTCCGGCTCAGGTGTCAGGTTGGTTT
TTGAGACAGAGTCTTTCACTTAGCTTGGAATTCACTGGCCGTCGTTTTACAACGTCGTGACTGGGAAAACCCTGGCGTTA
CCCAACTTAATCGCCTTGCAGCACATCCCCCTTTCGCCAGCTGGCGTAATAGCGAAGAGGCCCGCACCGATCGCCCTTCC
CAACAGTTGCGCAGCCTGAATGGCGAATGGCGCCTGATGCGGTATTTTCTCCTTACGCATCTGTGCGGTATTTCACACCG
CATATGGTGCACTCTCAGTACAATCTGCTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCGTCTCCGGGAGCTGCATGTGTCAGAGG
TTTTCACCGTCATCACCGAAACGCGCGATGACGAAAGGGCCTCGTGATACGCCTATTTTTATAGGTTAATGTCATGATAA
TAATGGTTTCTTAGACGTCAGGTGGCACTTTTCGGGGAAATGTGCGCGGAACCCCTATTTGTTTATTTTTCTAAATACAT
TCAAATATGTATCCGCTCATGAGACAATAACCCTGATAAATGCTTCAATAATATTGAAAAAGGAAGAGTATGAGTATTCA
ACATTTCCGTGTCGCCCTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGGTAAGATCCTTGAGAGT
TTTCGCCCCGAAGAACGTTTTCCAATGATGAGCACTTTTAAAGTTCTGCTATGTGGCGCGGTATTATCCCGTATTGACGC
CGGGCAAGAGCAACTCGGTCGCCGCATACACTATTCTCAGAATGACTTGGTTGAGTACTCACCAGTCACAGAAAAGCATC
TTACGGATGGCATGACAGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGATGGAGGCGGATAAAGTTGCAGGACCA
CTTCTGCGCTCGGCCCTTCCGGCTGGCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGACGGGGAGTCAGGCAACTATGGATGAAC
GAAATAGACAGATCGCTGAGATAGGTGCCTCACTGATTAAGCATTGGTAACTGTCAGACCAAGTTTACTCATATATACTT
TAGATTGATTTAAAACTTCATTTTTAATTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCATTGAGAAAGCGCCACGCTTCCCGAAGG
GAGAAAGGCGGACAGGTATCCGGTAAGCGGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTTGTGATGCTCGTCAGGGGGGCGGAGC
CTATGGAAAAACGCCAGCAACGCGGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCGCTCGCCGCAGCCGAACGACCGAGC
GCAGCGAGTCAGTGAGCGAGGAAGCGGAAGAGCGCCCAATACGCAAACCGCCTCTCCCCGCGCGTTGGCCGATTCATTAA
TGCAGCTGGCACGACAGGTTTCCCGACTGGAAAGCGGGCAGTGAGCGCAACGCAATTAATGTGAGTTAGCTCACTCATTA
GGCACCCCAGGCTTTACACTTTATGCTTCCGGCTCGTATGTTGTGTGGAATTGTGAGCGGATAACAATTTCACACAGGAA
ACAGCTATGACCATGATTACGCCAAGCTTTGCTCTTAGGAGTTTCCTAATACATCCCAAACTCAAATATATAAAGCATTT
GACTTGTTCTATGCCCTAGGGGGCGGGGGGAAGCTAAGCCAGCTTTTTTTAACATTTAAAATGTTAATTCCATTTTAAAT
GCACAGATGTTTTTATTTCATAAGGGTTTCAATGTGCATGAATGCTGCAATATTCCTGTTACCAAAGCTAGTATAAATAA
AAATAGATAAACGTGGAAATTACTTAGAGTTTCTGTCATTAACGTTTCCTTCCTCAGTTGACAACATAAATGCGCTGCTG
AGCAAGCCAGTTTGCATCTGTCAGGATCAATTTCCCATTATGCCAGTCATATTAATTACTAGTCAATTAGTTGATTTTTA
TTTTTGACATATACATGTGAATGAAAGACCCCACCTGTAGGTTTGGCAAGCTAGCTTAAGTAACGCCATTTTGCAAGGCA
TGGAAAAATACATAACTGAGAATAGAAAAGTTCAGATCAAGGTCAGGAACAGATGGAACAGCTGAATATGGGCCAAACAG
GATATCTGTGGTAAGCAGTTCCTGCCCCGGCTCAGGGCCAAGAACAGATGGAACAGCTGAATATGGGCCAAACAGGATAT
CTGTGGTAAGCAGTTCCTGCCCCGGCTCAGGGCCAAGAACAGATGGTCCCCAGATGCGGTCCAGCCCTCAGCAGTTTCTA
GAGAACCATCAGATGTTTCCAGGGTGCCCCAAGGACCTGAAATGACCCTGTGCCTTATTTGAACTAACCAATCAGTTCGC
TTCTCGCTTCTGTTCGCGCGCTTATGCTCCCCGAGCTCAATAAAAGAGCCCACAACCCCTCACTCGGGGCGCCAGTCCTC
CGATTGACTGAGTCGCCCGGGTACCCGTGTATCCAATAAACCCTCTTGCAGTTGCATCCGACTTGTGGTCTCGCTGTTCC
TTGGGAGGGTCTCCTCTGAGTGATTGACTACCCGTCAGCGGGGGTCTTTCATTTGGGGGCTCGTCCGGGATCGGGAGACC
CCTGCCCAGGGACCACCGACCCACCACCGGGAGGTAAGCTGGCCAGCAACTTATCTGTGTCTGTCCGATTGTCTAGTGTC
TATGACTGATTTTATGCGCCTGCGTCGGTACTAGTTAGCTAACTAGCTCTGTATCTGGCGGACCCGTGGTGGAACTGACG
AGTTCGGAACACCCGGCCGCAACCCTGGGAGACGTCCCAGGGACTTCGGGGGCCGTTTTTGTGGCCCGACCTGAGTCCTA
AAATCCCGATCGTTTAGGACTCTTTGGTGCACCCCCCTTAGAGGAGGGATATGTGGTTCTGGTAGGAGACGAGAACCTAA
AACAGTTCCCGCCTCCGTCTGAATTTTTGCTTTCGGTTTGGGACCGAAGCCGCGCCGCGCGTCTTGTCTGCTGCAGCATC
GTTCTGTGTTGTCTCTGTCTGACTGTGTTTCTGTATTTGTCTGAAAATATGGGCCCGGGCTAGACTGTTACCACTCCCTT
AAGTTTGACCTTAGGTCACTGGAAAGATGTCGAGCGGATCGCTCACAACCAGTCGGTAGATGTCAAGAAGAGACGTTGGG
TTACCTTCTGCTCTGCAGAATGGCCAACCTTTAACGTCGGATGGCCGCGAGACGGCACCTTTAACCGAGACCTCATCACC
CAGGTTAAGATCAAGGTCTTTTCACCTGGCCCGCATGGACACCCAGACCAGGTCCCCTACATCGTGACCTGGGAAGCCTT
GGCTTTTGACCCCCCTCCCTGGGTCAAGCCCTTTGTACACCCTAAGCCTCCGCCTCCTCTTCCTCCATCCGCCCCGTCTC
TCCCCCTTGAACCTCCTCGTTCGACCCCGCCTCGATCCTCCCTTTATCCAGCCCTCACTCCTTCTCTAGGCGCCCCCATA
TGGCCATATGAGATCTTATATGGGGCACCCCCGCCCCTTGTAAACTTCCCTGACCCTGACATGACAAGAGTTACTAACAG
CCCCTCTCTCCAAGCTCACTTACAGGCTCTCTACTTAGTCCAGCACGAAGTCTGGAGACCTCTGGCGGCAGCCTACCAAG
AACAACTGGACCGA.

The isolated nucleic acid molecule having the nucleotide sequence of SEQ ID NO:957 encodes a FcRL-5-targeted CAR (designated as 105 FcRL5-targeted BBz CAR) comprising a fully human scFv (encoded by nucleotides 207-1019) that comprises a heavy chain variable region comprising amino acids having the sequence set forth in SEQ ID NO:220, a light chain variable region comprising amino acids having the sequence set forth in SEQ ID NO:219, and a linker having an amino acid sequence of SEQ ID NO:307 positioned between the heavy chain variable region and the light chain variable region, a transmembrane domain comprising a CD8 polypeptide having 137 to 207 of SEQ ID NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 207-1019 of SEQ ID NO: 957 encodes the human scFv. Nucleotides 1029-1241 of SEQ ID NO: 957 encodes the CD8 polypeptide comprised in the transmembrane domain. Nucleotides 1242-1367 of SEQ ID NO: 957 encodes the 4-1BB polypeptide comprised in the intracellular domain. Nucleotides 1368-1706 of SEQ ID NO: 957 encodes the CD3zeta polypeptide comprised in the intracellular domain. Other portions of SEQ ID NO: 957 are shown in Table 236.

TABLE 236

| | Nucleotide Sequence | |
|---|---|---|
| | Positions of SEQ ID NO: | |
| Portions | 957 | Number of Nucleotides |
| antiFcRL5 scFv 105 | 207 . . . 1019 | 813 |
| CD8a TM | 1029 . . . 1241 | 213 |
| 4-1BB | 1242 . . . 1367 | 126 |
| CD3zeta | 1368 . . . 1706 | 339 |
| LTR | 1986 . . . 2455 | 470 |
| M13 fwd | 3154 . . . 3170 | 17 |
| AmpR promoter | 3645 . . . 3749 | 105 |
| AmpR | 3750 . . . 4610 | 861 |
| ori | 4781 . . . 5369 | 589 |
| CAP binding site | 5657 . . . 5678 | 22 |
| lac promoter | 5693 . . . 5723 | 31 |
| lac operator | 5731 . . . 5747 | 17 |
| M13 rev | 5755 . . . 5771 | 17 |
| LTR | 6180 . . . 6773 | 594 |
| MMLV Psi | 6836 . . . 7193 | 358 |
| gag (truncated) | 7258 . . . 7674 | 417 |

In certain embodiments, the isolated nucleic acid molecule comprises nucleic acids having the sequence set forth in SEQ ID NO:958 provided below:

[SEQ ID NO: 958]
CCGGTGGTACCTCACCCTTACCGAGTCGGCGACACAGTGTGGGTCCGCCGACACCAGACTAAGAACCTAGAACCTCGCTG
GAAAGGACCTTACACAGTCCTGCTGACCACCCCCACCGCCCTCAAAGTAGACGGCATCGCAGCTTGGATACACGCCGCCC
ACGTGAAGGCTGCCGACCCCGGGGGTGGACCATCCTCTAGACTGCCATGGAAACCGACACCCTGCTGCTGTGGGTGCTGC
TGCTGTGGGTGCCAGGATCCACAGGACTGCCTGTGCTGACTCAGCCACCCTCAGCGTCTGCGACCCCCGGGCAGAGGGTC
ACCATCTCTTGTTCTGGAACCACCTCCAACATCGGAAGTAATACTGTACACTGGTACCAGCAGCTCCCAGGGACGGCCCC
CAAACTCCTCATCTATAATAATAATCAGCGGCCCTCAGGGGTCCCTGACCGATTCTCTGGCTCCAAGTCTGGCACCTCAG
CCTCCCTGGCCATCAGTGGGCTCCGGTCCGAGGATGAGGCTACATATTCCTGTGCAACATGGGATGACAGCCTGAGTGGT
GTGGTCTTCGGCGGAGGGACCAAGCTGACCGTCCTAGGTTCTAGAGGTGGTGGTGGTAGCGGCGGCGGCGGCTCTGGTGG
TGGTGGATCCCTCGAGATGGCCGAGGTCCAGCTGGTGCAGTCTGGGGCTGAGGTGAAGAAGCCTGGGTCCTCGGTGAAGG
TCTCCTGCAAGGCTTCTGGAGGCACCTTCAGCAGCTATGCTATCAGCTGGGTGCGACAGGCCCCTGGACAAGGGCTTGAG
TGGATGGGAGGGATCATCCCTATCTTTGGTACAGCAAACTACGCACAGAAGTTCCAGGGCAGAGTCACGATTACCGCGGA
CGAATCCACGAGCACAGCCTACATGGAGCTGAGCAGCCTGAGATCTGAGGACACGGCCGTGTATTACTGTGCGAGAGATC
CCGCCTACGGTGACTACGAGTATGATGCTTTTGATATCTGGGGCCAAGGGACAATGGTCACCGTCTCTTCAGCGGCCGCA
CCCACCACGACGCCAGCGCCGCGACCACCAACCCCGGCGCCCACGATCGCGTCGCAGCCCCTGTCCCTGCGCCCAGAGGC
GTGCCGGCCAGCGGCGGGGGGCGCAGTGCACACGAGGGGGCTGGACTTCGCCTGTGATATCTACATCTGGGCGCCCCTGG
CCGGGACTTGTGGGGTCCTTCTCCTGTCACTGGTTATCACCCTTTACTGCAACAAACGGGGCAGAAAGAAGCTCCTGTAT
ATATTCAAACAACCATTTATGAGACCAGTACAAACTACTCAAGAGGAAGATGGCTGTAGCTGCCGATTTCCAGAAGAAGA
AGAAGGAGGATGTGAACTGAGAGTGAAGTTCAGCAGGAGCGCAGAGCCCCCCGCGTACCAGCAGGGCCAGAACCAGCTCT
ATAACGAGCTCAATCTAGGACGAAGAGAGGAGTACGATGTTTTGGACAAGAGACGTGGCCGGGACCCTGAGATGGGGGGA
AAGCCGAGAAGGAAGAACCCTCAGGAAGGCCTGTACAATGAACTGCAGAAAGATAAGATGGCGGAGGCCTACAGTGAGAT
TGGGATGAAAGGCGAGCGCCGGAGGGGCAAGGGGCACGATGGCCTTTACCAGGGTCTCAGTACAGCCACCAAGGACACCT
ACGACGCCCTTCACATGCAGGCCCTGCCCCCTCGCTAACAGCCACTCGAGGATCCGGATTAGTCCAATTTGTTAAAGACA
GGATATCAGTGGTCCAGGCTCTAGTTTTGACTCAACAATATCACCAGCTGAAGCCTATAGAGTACGAGCCATAGATAAAA
TAAAAGATTTTATTTAGTCTCCAGAAAAAGGGGGGAATGAAAGACCCCACCTGTAGGTTTGGCAAGCTAGCTTAAGTAAC
GCCATTTTGCAAGGCATGGAAAAATACATAACTGAGAATAGAGAAGTTCAGATCAAGGTCAGGAACAGATGGAACAGCTG
AATATGGGCCAAACAGGATATCTGTGGTAAGCAGTTCCTGCCCCGGCTCAGGGCCAAGAACAGATGGAACAGCTGAATAT
GGGCCAAACAGGATATCTGTGGTAAGCAGTTCCTGCCCCGGCTCAGGGCCAAGAACAGATGGTCCCCAGATGCGGTCCAG
CCCTCAGCAGTTTCTAGAGAACCATCAGATGTTTCCAGGGTGCCCCAAGGACCTGAAATGACCCTGTGCCTTATTTGAAC
TAACCAATCAGTTCGCTTCTCGCTTCTGTTCGCGCGCTTCTGCTCCCCGAGCTCAATAAAAGAGCCCACAACCCCTCACT
CGGGGCGCCAGTCCTCCGATTGACTGAGTCGCCCGGGTACCCGTGTATCCAATAAACCCTCTTGCAGTTGCATCCGACTT
GTGGTCTCGCTGTTCCTTGGGAGGGTCTCCTCTGAGTGATTGACTACCCGTCAGCGGGGGTCTTTCACACATGCAGCATG
TATCAAAATTAATTTGGTTTTTTTTCTTAAGTATTTACATTAAATGGCCATAGTACTTAAAGTTACATTGGCTTCCTTGA
AATAAACATGGAGTATTCAGAATGTGTCATAAATATTTCTAATTTTAAGATAGTATCTCCATTGGCTTTCTACTTTTTCT
TTTATTTTTTTTTGTCCTCTGTCTTCCATTTGTTGTTGTTGTTGTTTGTTTGTTTGTTTGTTGGTTGGTTGGTTAATTTT
TTTTTAAAGATCCTACACTATAGTTCAAGCTAGACTATTAGCTACTCTGTAACCCAGGGTGACCTTGAAGTCATGGGTAG
CCTGCTGTTTTAGCCTTCCCACATCTAAGATTACAGGTATGAGCTATCATTTTTGGTATATTGATTGATTGATTGATTGA
TGTGTGTGTGTGTGATTGTGTTTGTGTGTGTGACTGTGAAAATGTGTGTATGGGTGTGTGTGAATGTGTGTATGTATGTG
TGTGTGTGAGTGTGTGTGTGTGTGTGTGCATGTGTGTGTGTGTGACTGTGTCTATGTGTATGACTGTGTGTGTGTGTGTG
TGTGTGTGTGTGTGTGTGTGTGTGTGTGTGTTGTGAAAAAATATTCTATGGTAGTGAGAGCCAACGCTCCGGCTCAGGTG
TCAGGTTGGTTTTTGAGACAGAGTCTTTCACTTAGCTTGGAATTCACTGGCCGTCGTTTTACAACGTCGTGACTGGGAAA
ACCCTGGCGTTACCCAACTTAATCGCCTTGCAGCACATCCCCCTTTCGCCAGCTGGCGTAATAGCGAAGAGGCCCGCACC
GATCGCCCTTCCCAACAGTTGCGCAGCCTGAATGGCGAATGGCGCCTGATGCGGTATTTTCTCCTTACGCATCTGTGCGG
TATTTCACACCGCATATGGTGCACTCTCAGTACAATCTGCTCTGATGCCGCATAGTTAAGCCAGCCCCGACACCCGCCAA
CACCCGCTGACGCGCCCTGACGGGCTTGTCTGCTCCCGGCATCCGCTTACAGACAAGCTGTGACCGTCTCCGGGAGCTGC
ATGTGTCAGAGGTTTTCACCGTCATCACCGAAACGCGCGATGACGAAAGGGCCTCGTGATACGCCTATTTTTATAGGTTA
ATGTCATGATAATAATGGTTTCTTAGACGTCAGGTGGCACTTTTCGGGGAAATGTGCGCGGAACCCCTATTTGTTTATTT
TTCTAAATACATTCAAATATGTATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATCGAACTGGATCTCAACAGCGGTAAG
ATCCTTGAGAGTTTTCGCCCCGAAGAACGTTTTCCAATGATGAGCACTTTTAAAGTTCTGCTATGTGGCGCGGTATTATC
CCGTATTGACGCCGGGCAAGAGCAACTCGGTCGCCGCATACACTATTCTCAGAATGACTTGGTTGAGTACTCACCAGTCA
CAGAAAAGCATCTTACGGATGGCATGACAGTAAGAGAATTATGCAGTGCTGCCATAACCATGAGTGATAACACTGCGGCC
AACTTACTTCTGACAACGATCGGAGGACCGAAGGAGCTAACCGCTTTTTTGCACAACATGGGGGATCATGTAACTCGCCT
TGATCGTTGGGAACCGGAGCTGAATGAAGCCATACCAAACGACGAGCGTGACACCACGATGCCTGTAGCAATGGCAACAA
CGTTGCGCAAACTATTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCCGGTGAGCGTGGGTC
TCGCGGTATCATTGCAGCACTGGGGCCAGATGGTAAGCCCTCCCGTATCGTAGTTATCTACACGACGGGGAGTCAGGCAA
CTATGGATGAACGAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACTGTCCTTCTAGTGTAGCCGTAGTT
AGGCCACCACTTCAAGAACTCTGTAGCACCGCCTACATACCTCGCTCTGCTAATCCTGTTACCAGTGGCTGCTGCCAGTG
GCGATAAGTCGTGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCATTGAGAAAGCGCCAC
GCTTCCCGAAGGGAGAAAGGCGGACAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTTCCTGGCCTTTTGCTGGCCTTTTGCTCACAT
GTTCTTTCCTGCGTTATCCCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCCAATACGCAAACCGCCTCTCCCCGCGCGTTGG
CCGATTCATTAATGCAGCTGGCACGACAGGTTTCCCGACTGGAAAGCGGGCAGTGAGCGCAACGCAATTAATGTGAGTTA
GCTCACTCATTAGGCACCCCAGGCTTTACACTTTATGCTTCCGGCTCGTATGTTGTGTGGAATTGTGAGCGGATAACAAT
TTCACACAGGAAACAGCTATGACCATGATTACGCCAAGCTTTGCTCTTAGGAGTTTCCTAATACATCCCAAACTCAAATA
TATAAAGCATTTGACTTGTTCTATGCCCTAGGGGGCGGGGGGAAGCTAAGCCAGCTTTTTTTAACATTTAAAATGTTAAT
TCCATTTTAAATGCACAGATGTTTTTATTTCATAAGGGTTTCAATGTGCATGAATGCTGCAATATTCCTGTTACCAAAGC
TAGTATAAATAAAAATAGATAAACGTGGAAATTACTTAGAGTTTCTGTCATTAACGTTTCCTTCCTCAGTTGACAACATA
AATGCGCTGCTGAGCAAGCCAGTTTGCATCTGTCAGGATCAATTTCCCATTATGCCAGTCATATTAATTACTAGTCAATT
AGTTGATTTTTATTTTTGACATATACATGTGAATGAAAGACCCCACCTGTAGGTTTGGCAAGCTAGCTTAAGTAACGCCA
TTTTGCAAGGCATGGAAAAATACATAACTGAGAATAGAAAAGTTCAGATCAAGGTCAGGAACAGATGGAACAGCTGAATA
TGGGCCAAACAGGATATCTGTGGTAAGCAGTTCCTGCCCCGGCTCAGGGCCAAGAACAGATGGAACAGCTGAATATGGGC
CAAACAGGATATCTGTGGTAAGCAGTTCCTGCCCCGGCTCAGGGCCAAGAACAGATGGTCCCCAGATGCGGTCCAGCCCT
CAGCAGTTTCTAGAGAACCATCAGATGTTTCCAGGGTGCCCCAAGGACCTGAAATGACCCTGTGCCTTATTTGAACTAAC
CAATCAGTTCGCTTCTCGCTTCTGTTCGCGCGCTTATGCTCCCCGAGCTCAATAAAAGAGCCCACAACCCCTCACTCGGG
GCGCCAGTCCTCCGATTGACTGAGTCGCCCGGGTACCCGTGTATCCAATAAACCCTCTTGCAGTTGCATCCGACTTGTGG
TCTCGCTGTTCCTTGGGAGGGTCTCCTCTGAGTGATTGACTACCCGTCAGCGGGGGTCTTTCATTTGGGGGCTCGTCCGG
GATCGGGAGACCCCTGCCCAGGGACCACCGACCCACCACCGGGAGGTAAGCTGGCCAGCAACTTATCTGTGTCTGTCCGA
TTGTCTAGTGTCTATGACTGATTTTATGCGCCTGCGTCGGTACTAGTTAGCTAACTAGCTCTGTATCTGGCGGACCCGTG
GTGGAACTGACGAGTTCGGAACACCCGGCCGCAACCCTGGGAGACGTCCCAGGGACTTCGGGGGCCGTTTTTGTGGCCCG
ACCTGAGTCCTAAAATCCCGATCGTTTAGGACTCTTTGGTGCACCCCCCTTAGAGGAGGGATATGTGGTTCTGGTAGGAG
ACGAGAACCTAAAACAGTTCCCGCCTCCGTCTGAATTTTTGCTTTCGGTTTGGGACCGAAGCCGCGCCGCGCGTCTTGTC
TGCTGCAGCATCGTTCTGTGTTGTCTCTGTCTGACTGTGTTTCTGTATTTGTCTGAAAATATGGGCCCGGGCTAGACTGT
TACCACTCCCTTAAGTTTGACCTTAGGTCACTGGAAAGATGTCGAGCGGATCGCTCACAACCAGTCGGTAGATGTCAAGA
AGAGACGTTGGGTTACCTTCTGCTCTGCAGAATGGCCAACCTTTAACGTCGGATGGCCGCGAGACGGCACCTTTAACCGA
GACCTCATCACCCAGGTTAAGATCAAGGTCTTTTCACCTGGCCCGCATGGACACCCAGACCAGGTCCCCTACATCGTGAC
CTGGGAAGCCTTGGCTTTTGACCCCCCTCCCTGGGTCAAGCCCTTTGTACACCCTAAGCCTCCGCCTCCTCTTCCTCCAT
CCGCCCCGTCTCTCCCCCTTGAACCTCCTCGTTCGACCCCGCCTCGATCCTCCCTTTATCCAGCCCTCACTCCTTCTCTA
GGCGCCCCCATATGGCCATATGAGATCTTATATGGGGCACCCCCGCCCCTTGTAAACTTCCCTGACCCTGACATGACAAG
AGTTACTAACAGCCCCTCTCTCCAAGCTCACTTACAGGCTCTCTACTTAGTCCAGCACGAAGTCTGGAGACCTCTGGCGG
CAGCCTACCAAGAACAACTGGACCGA.

The isolated nucleic acid molecule having the nucleotide sequence of SEQ ID NO:958 encodes a FcRL-5-targeted CAR (designated as 109 FcRL5-targeted BBz CAR) comprising a fully human scFv (encoded by nucleotides 207-1031) that comprises a heavy chain variable region comprising amino acids having the sequence set forth in SEQ ID NO:236, a light chain variable region comprising amino acids having the sequence set forth in SEQ ID NO:235, and a linker having an amino acid sequence of SEQ ID NO:307 positioned between the heavy chain variable region and the light chain variable region, a transmembrane domain comprising a CD8 polypeptide having 137 to 207 of SEQ ID NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 207-1031 of SEQ ID NO: 957 encodes the human scFv. Nucleotides 1041-1253 of SEQ ID NO: 958 encodes the CD8 polypeptide comprised in the transmembrane domain. Nucleotides 1254-1379 of SEQ ID NO: 958 encodes the 4-1BB polypeptide comprised in the intracellular domain. Nucleotides 1380-1718 of SEQ ID NO: 958 encodes the CD3zeta polypeptide comprised in the intracellular domain. Other portions of SEQ ID NO: 958 are shown in Table 237.

TABLE 237

| | Nucleotide Sequence | |
|---|---|---|
| | Positions of SEQ ID | |
| Portions | NO: 958 | Number of Nucleotides |
| anti-FcRL5 scFv 109 | 207 . . . 1031 | 825 |
| CD8a TM | 1041 . . . 1253 | 213 |
| 4-1BB | 1254 . . . 1379 | 126 |
| CD3zeta | 1380 . . . 1718 | 339 |
| LTR | 1998 . . . 2467 | 470 |
| M13 fwd | 3166 . . . 3182 | 17 |
| AmpR promoter | 3657 . . . 3761 | 105 |
| AmpR | 3762 . . . 4622 | 861 |
| ori | 4793 . . . 5381 | 589 |
| CAP binding site | 5669 . . . 5690 | 22 |
| lac promoter | 5705 . . . 5735 | 31 |
| lac operator | 5743 . . . 5759 | 17 |
| M13 rev | 5767 . . . 5783 | 17 |
| LTR | 6192 . . . 6785 | 594 |
| MMLV Psi | 6848 . . . 7205 | 358 |
| gag (truncated) | 7270 . . . 7686 | 417 |

In certain embodiments, the isolated nucleic acid molecule comprises nucleic acids having the sequence set forth in SEQ ID NO:959 provided below:

[SEQ ID NO: 959]
CCGGTGGTACCTCACCCTTACCGAGTCGGCGACACAGTGTGGGTCCGCCGACACCAGACTAAGAACCTAGAACCTCGCTG
GAAAGGACCTTACACAGTCCTGCTGACCACCCCCACCGCCCTCAAAGTAGACGGCATCGCAGCTTGGATACACGCCGCCC
ACGTGAAGGCTGCCGACCCCGGGGGTGGACCATCCTCTAGACTGCCATGGAAACCGATACACTGCTGCTGTGGGTGCTGC
TGCTGTGGGTGCCAGGATCCACAGGCGATGTTGTGATGACTCAGTCTCCACCCTCCCTGTCCGTCACCCCTGGAGAGCCG
GCCTCCATCACCTGCAGGTCTAGTCAGAGCCTCCTGGAAAGAAATGCATACAACTACTTGGATTGGTACCTGCAGAGGCC
AGGACAGTCTCCACAGCTCCTGATCTACTTGGGTTCTAATCGGGCCGCCGGGGTCCCTGACAGGTTCAGTGGCAGTGGAT
CAGGCAGAGATTTTACACTGAAAATCAGCAGAGTGGAGCCTGAGGATGTTGGGGTTTATTACTGCATGCAAGCTCTACAA
GCTCCGTTCACTTTCGGCGGAGGGACCAAGGTGGAGATCAAACGTTCTAGAGGTGGTGGTGGTAGCGGCGGCGGCGGCTC
TGGTGGTGGTGGATCCCTCGAGATGGCCGAAGTGCAGCTGGTGCAGTCTGGGGGAGGCTTGGTACAGCCTGGGGGGTCCC
TGAGACTCTCCTGTGCAGCCTCTGGATTCACCTTTAGCAGCTATGCCATGAGCTGGGTCCGCCAGGCTCCAGGGAAGGGG
CTGGAGTGGGTCTCAGCTATTAGTGGTAGTGGTGGTAGCACATACTACGCAGACTCCGTGAAGGGCCGGTTCACCATCTC
CAGAGACAATTCCAAGAACACGCTGTATCTGCAAATGAACAGCCTGAGAGCCGAGGACACGGCCGTATATTACTGTGCGA
AATGGGGCCCGTTTCAGGATGCTTTTGATATCTGGGGCCAAGGGACAATGGTCACCGTCTCTTCAGCGGCCGCACCCACC
ACGACGCCAGCGCCGCGACCACCAACCCCGGCGCCCACGATCGCGTCGCAGCCCCTGTCCCTGCGCCCAGAGGCGTGCCG
GCCAGCGGCGGGGGGCGCAGTGCACACGAGGGGGCTGGACTTCGCCTGTGATATCTACATCTGGGCGCCCCTGGCCGGGA
CTTGTGGGGTCCTTCTCCTGTCACTGGTTATCACCCTTTACTGCAACAAACGGGGCAGAAAGAAGCTCCTGTATATATTC
AAACAACCATTTATGAGACCAGTACAAACTACTCAAGAGGAAGATGGCTGTAGCTGCCGATTTCCAGAAGAAGAAGAAGG
AGGATGTGAACTGAGAGTGAAGTTCAGCAGGAGCGCAGAGCCCCCCGCGTACCAGCAGGGCCAGAACCAGCTCTATAACG
AGCTCAATCTAGGACGAAGAGAGGAGTACGATGTTTTGGACAAGAGACGTGGCCGGGACCCTGAGATGGGGGGAAAGCCG
AGAAGGAAGAACCCTCAGGAAGGCCTGTACAATGAACTGCAGAAAGATAAGATGGCGGAGGCCTACAGTGAGATTGGGAT
GAAAGGCGAGCGCCGGAGGGGCAAGGGGCACGATGGCCTTTACCAGGGTCTCAGTACAGCCACCAAGGACACCTACGACG
CCCTTCACATGCAGGCCCTGCCCCCTCGCTAACAGCCACTCGAGGATCCGGATTAGTCCAATTTGTTAAAGACAGGATAT
CAGTGGTCCAGGCTCTAGTTTTGACTCAACAATATCACCAGCTGAAGCCTATAGAGTACGAGCCATAGATAAAATAAAAG
ATTTTATTTAGTCTCCAGAAAAAGGGGGGAATGAAAGACCCCACCTGTAGGTTTGGCAAGCTAGCTTAAGTAACGCCATT
TTGCAAGGCATGGAAAAATACATAACTGAGAATAGAGAAGTTCAGATCAAGGTCAGGAACAGATGGAACAGCTGAATATG
GGCCAAACAGGATATCTGTGGTAAGCAGTTCCTGCCCCGGCTCAGGGCCAAGAACAGATGGAACAGCTGAATATGGGCCA
AACAGGATATCTGTGGTAAGCAGTTCCTGCCCCGGCTCAGGGCCAAGAACAGATGGTCCCCAGATGCGGTCCAGCCCTCA
GCAGTTTCTAGAGAACCATCAGATGTTTCCAGGGTGCCCCAAGGACCTGAAATGACCCTGTGCCTTATTTGAACTAACCA
ATCAGTTCGCTTCTCGCTTCTGTTCGCGCGCTTCTGCTCCCCGAGCTCAATAAAAGAGCCCACAACCCCTCACTCGGGGC
GCCAGTCCTCCGATTGACTGAGTCGCCCGGGTACCCGTGTATCCAATAAACCCTCTTGCAGTTGCATCCGACTTGTGGTC
TCGCTGTTCCTTGGGAGGGTCTCCTCTGAGTGATTGACTACCCGTCAGCGGGGGTCTTTCACACATGCAGCATGTATCAA
AATTAATTTGGTTTTTTTTCTTAAGTATTTACATTAAATGGCCATAGTACTTAAAGTTACATTGGCTTCCTTGAAATAAA
CATGGAGTATTCAGAATGTGTCATAAATATTTCTAATTTTAAGATAGTATCTCCATTGGCTTTCTACTTTTTCTTTTATT
TTTTTTTGTCCTCTGTCTTCCATTTGTTGTTGTTGTTGTTTGTTTGTTTGTTTGTTGGTTGGTTGGTTAATTTTTTTTTA
AAGATCCTACACTATAGTTCAAGCTAGACTATTAGCTACTCTGTAACCCAGGGTGACCTTGAAGTCATGGGTAGCCTGCT
GTTTTAGCCTTCCCACATCTAAGATTACAGGTATGAGCTATCATTTTTGGTATATTGATTGATTGATTGATTGATGTGTG
TGTGTGTGATTGTGTTTGTGTGTGTGACTGTGAAAATGTGTGTATGGGTGTGTGTGAATGTGTGTATGTATGTGTGTGTG
TGAGTGTGTGTGTGTGTGTGTGCATGTGTGTGTGTGTGACTGTGTCTATGTGTATGACTGTGTGTGTGTGTGTGTGTGTG
TGTGTGTGTGTGTGTGTGTGTGTGTTGTGAAAAAATATTCTATGGTAGTGAGAGCCAACGCTCCGGCTCAGGTGTCAGGT
TGGTTTTTGAGACAGAGTCTTTCACTTAGCTTGGAATTCACTGGCCGTCGTTTTACAACGTCGTGACTGGGAAAACCCTG
GCGTTACCCAACTTAATCGCCTTGCAGCACATCCCCCTTTCGCCAGCTGGCGTAATAGCGAAGAGGCCCGCACCGATCGC
CCTTCCCAACAGTTGCGCAGCCTGAATGGCGAATGGCGCCTGATGCGGTATTTTCTCCTTACGCATCTGTGCGGTATTTC
ACACCGCATATGGTGCACTCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTCTCCGGGAGCTGCATGTGT
CAGAGGTTTTCACCGTCATCACCGAAACGCGCGATGACGAAAGGGCCTCGTGATACGCCTATTTTTATAGGTTAATGTCA
TGATAATAATGGTTTCTTAGACGTCAGGTGGCACTTTTCGGGGAAATGTGCGCGGAACCCCTATTTGTTTATTTTTCTAA
ATACATTCAAATATGTATCCGCTCATGAGACAATAACCCTGATAAATGCTTCAATAATATTGAAAAAGGAAGAGTATGAG
TATTCAACATTTCCGTGTCGCCCTTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAACTGGATCTCAACAGCGGTAAGATCCTT
GAGAGTTTTCGCCCCGAAGAACGTTTTCCAATGATGAGCACTTTTAAAGTTCTGCTATGTGGCGCGGTATTATCCCGTAT
TGACGCCGGGCAAGAGCAACTCGGTCGCCGCATACACTATTCTCAGAATGACTTGGTTGAGTACTCACCAGTCACAGAAA
AGCATCTTACGGATGGCATGACAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGATCATGTAACTCGCCTTGATCG
TTGGGAACCGGAGCTGAATGAAGCCATACCAAACGACGAGCGTGACACCACGATGCCTGTAGCAATGGCAACAACGTTGC
GCAAACTATTAACTGGCGAACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCTTCTTGAGATCCTTTTT
TTCTGCGCGTAATCTGCTGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCCTTCTAGTGTAGCCGTAGTTAGGCCA
CCACTTCAAGAACTCTGTAGCACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCATTGAGAAAGCGCCACGCTTCC
CGAAGGGAGAAAGGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCCAATACGCAAACCGCCTCTCCCCGCGCGTTGGCCGATT
CATTAATGCAGCTGGCACGACAGGTTTCCCGACTGGAAAGCGGGCAGTGAGCGCAACGCAATTAATGTGAGTTAGCTCAC
TCATTAGGCACCCCAGGCTTTACACTTTATGCTTCCGGCTCGTATGTTGTGTGGAATTGTGAGCGGATAACAATTTCACA
CAGGAAACAGCTATGACCATGATTACGCCAAGCTTTGCTCTTAGGAGTTTCCTAATACATCCCAAACTCAAATATATAAA
GCATTTGACTTGTTCTATGCCCTAGGGGGCGGGGGGAAGCTAAGCCAGCTTTTTTTAACATTTAAAATGTTAATTCCATT
TTAAATGCACAGATGTTTTTATTTCATAAGGGTTTCAATGTGCATGAATGCTGCAATATTCCTGTTACCAAAGCTAGTAT
AAATAAAAATAGATAAACGTGGAAATTACTTAGAGTTTCTGTCATTAACGTTTCCTTCCTCAGTTGACAACATAAATGCG
CTGCTGAGCAAGCCAGTTTGCATCTGTCAGGATCAATTTCCCATTATGCCAGTCATATTAATTACTAGTCAATTAGTTGA
TTTTTATTTTTGACATATACATGTGAATGAAAGACCCCACCTGTAGGTTTGGCAAGCTAGCTTAAGTAACGCCATTTTGC
AAGGCATGGAAAAATACATAACTGAGAATAGAAAAGTTCAGATCAAGGTCAGGAACAGATGGAACAGCTGAATATGGGCC
AAACAGGATATCTGTGGTAAGCAGTTCCTGCCCCGGCTCAGGGCCAAGAACAGATGGAACAGCTGAATATGGGCCAAACA
GGATATCTGTGGTAAGCAGTTCCTGCCCCGGCTCAGGGCCAAGAACAGATGGTCCCCAGATGCGGTCCAGCCCTCAGCAG
TTTCTAGAGAACCATCAGATGTTTCCAGGGTGCCCCAAGGACCTGAAATGACCCTGTGCCTTATTTGAACTAACCAATCA
GTTCGCTTCTCGCTTCTGTTCGCGCGCTTATGCTCCCCGAGCTCAATAAAAGAGCCCACAACCCCTCACTCGGGGCGCCA
GTCCTCCGATTGACTGAGTCGCCCGGGTACCCGTGTATCCAATAAACCCTCTTGCAGTTGCATCCGACTTGTGGTCTCGC
TGTTCCTTGGGAGGGTCTCCTCTGAGTGATTGACTACCCGTCAGCGGGGGTCTTTCATTTGGGGGCTCGTCCGGGATCGG
GAGACCCCTGCCCAGGGACCACCGACCCACCACCGGGAGGTAAGCTGGCCAGCAACTTATCTGTGTCTGTCCGATTGTCT
AGTGTCTATGACTGATTTTATGCGCCTGCGTCGGTACTAGTTAGCTAACTAGCTCTGTATCTGGCGGACCCGTGGTGGAA
CTGACGAGTTCGGAACACCCGGCCGCAACCCTGGGAGACGTCCCAGGGACTTCGGGGGCCGTTTTTGTGGCCCGACCTGA
GTCCTAAAATCCCGATCGTTTAGGACTCTTTGGTGCACCCCCCTTAGAGGAGGGATATGTGGTTCTGGTAGGAGACGAGA
ACCTAAAACAGTTCCCGCCTCCGTCTGAATTTTTGCTTTCGGTTTGGGACCGAAGCCGCGCCGCGCGTCTTGTCTGCTGC
AGCATCGTTCTGTGTTGTCTCTGTCTGACTGTGTTTCTGTATTTGTCTGAAAATATGGGCCCGGGCTAGACTGTTACCAC
TCCCTTAAGTTTGACCTTAGGTCACTGGAAAGATGTCGAGCGGATCGCTCACAACCAGTCGGTAGATGTCAAGAAGAGAC
GTTGGGTTACCTTCTGCTCTGCAGAATGGCCAACCTTTAACGTCGGATGGCCGCGAGACGGCACCTTTAACCGAGACCTC
ATCACCCAGGTTAAGATCAAGGTCTTTTCACCTGGCCCGCATGGACACCCAGACCAGGTCCCCTACATCGTGACCTGGGA
AGCCTTGGCTTTTGACCCCCCTCCCTGGGTCAAGCCCTTTGTACACCCTAAGCCTCCGCCTCCTCTTCCTCCATCCGCCC
CGTCTCTCCCCCTTGAACCTCCTCGTTCGACCCCGCCTCGATCCTCCCTTTATCCAGCCCTCACTCCTTCTCTAGGCGCC
CCCATATGGCCATATGAGATCTTATATGGGGCACCCCCGCCCCTTGTAAACTTCCCTGACCCTGACATGACAAGAGTTAC
TAACAGCCCCTCTCTCCAAGCTCACTTACAGGCTCTCTACTTAGTCCAGCACGAAGTCTGGAGACCTCTGGCGGCAGCCT
ACCAAGAACAACTGGACCGA.

The isolated nucleic acid molecule having the nucleotide sequence of SEQ ID NO:959 encodes a FcRL-5-targeted CAR (designated as 117 FcRL5-targeted BBz CAR) comprising a fully human scFv (encoded by nucleotides 207-1025 of SEQ ID NO:959) that comprises a heavy chain variable region comprising amino acids having the sequence set forth in SEQ ID NO:268, a light chain variable region comprising amino acids having the sequence set forth in SEQ ID NO:267, and a linker having an amino acid sequence of SEQ ID NO:307 positioned between the heavy chain variable region and the light chain variable region, a transmembrane domain comprising a CD8 polypeptide having 137 to 207 of SEQ ID NO: 960, and an intracellular domain comprising a CD3 polypeptide comprising amino acids 52 to 163 of SEQ ID NO: 941, and a co-stimulatory signaling region comprising a 4-1BB polypeptide having amino acids 214-255 of SEQ ID NO: 943. Nucleotides 207-1025 of SEQ ID NO: 959 encodes the human scFv. Nucleotides 1035-1247 of SEQ ID NO: 959 encodes the CD8 polypeptide comprised in the transmembrane domain. Nucleotides 1248-1373 of SEQ ID NO: 959 encodes the 4-1BB polypeptide comprised in the intracellular domain. Nucleotides 1374-1712 of SEQ ID NO: 959 encodes the CD3zeta polypeptide comprised in the intracellular domain. Other portions of SEQ ID NO: 959 are shown in Table 238.

TABLE 238

| | Nucleotide Sequence | |
|---|---|---|
| | Positions of SEQ ID | |
| Portions | NO: 959 | Number of Nucleotides |
| anti-FcRL5 scFv 117 | 207 . . . 1025 | 819 |
| CD8a TM | 1035 . . . 1247 | 213 |
| 4-1BB | 1248 . . . 1373 | 126 |
| CD3zeta | 1374 . . . 1712 | 339 |
| LTR | 1992 . . . 2461 | 470 |
| M13 fwd | 3160 . . . 3176 | 17 |
| AmpR promoter | 3651 . . . 3755 | 105 |
| AmpR | 3756 . . . 4616 | 861 |
| ori | 4787 . . . 5375 | 589 |
| CAP binding site | 5663 . . . 5684 | 22 |
| lac promoter | 5699 . . . 5729 | 31 |
| lac operator | 5737 . . . 5753 | 17 |
| M13 rev | 5761 . . . 5777 | 17 |
| LTR | 6186 . . . 6779 | 594 |
| MMLV Psi | 6842 . . . 7199 | 358 |
| gag (truncated) | 7264 . . . 7680 | 417 |

In certain embodiments, the isolated nucleic acid molecule encodes a functional portion of a presently disclosed FcRL5-targeted CAR. As used herein, the term “functional portion” refers to any portion, part or fragment of a presently disclosed FcRL5-targeted CAR, which portion, part or fragment retains the biological activity of the FcRL5-targeted CAR (the parent CAR). For example, functional portions encompass the portions, parts or fragments of a presently disclosed FcRL5-targeted CAR that retains the ability to recognize a target cell, to treat a disease, e.g., multiple myeloma, to a similar, same, or even a higher extent as the parent CAR. In certain embodiments, an isolated nucleic acid molecule encoding a functional portion of a presently disclosed FcRL5-targeted CAR can encode a protein comprising, e.g., about 10%, 20%, 25%, 30%, 35%, 40%, 45%, 50%, 55%, 60%, 65%, 70%, 75%, 80%, 85%, 90%, and 95%, or more of the parent CAR, e.g., of the nucleic acid sequences set forth in SEQ ID NO:951, SEQ ID NO:952, SEQ ID NO:953, SEQ ID NO:954, SEQ ID NO:955, SEQ ID NO:956, SEQ ID NO:957, SEQ ID NO:958 or SEQ ID NO:959.

### III. Immunoresponsive Cells

The presently disclosed subject matter provides immunoresponsive cells expressing a CAR that comprises an extracellular antigen-binding domain, a transmembrane domain and an intracellular domain, where the extracellular antigen-binding domain specifically binds to FcRL5 (e.g., human FcRL5), as described above. In certain embodiments, the extracellular antigen-binding domain specifically binds to domain 7 of FcRL5. In certain embodiments, the extracellular antigen-binding domain specifically binds to domain 8 of FcRL5. In certain embodiments, the extracellular antigen-binding domain specifically binds to domain 9 of FcRL5. The immunoresponsive cells can be transduced with a presently disclosed CAR such that the cells express the CAR. The presently disclosed subject matter also provides methods of using such cells for the treatment of a tumor, e.g., multiple myeloma (MM). The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, cytotoxic T lymphocytes (CTLs), regulatory T cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells), Regulatory T cells (also known as suppressor T cells), Natural killer T cells, Mucosal associated invariant T cells, and T6 T cells. In certain embodiments, the CAR-expressing T cells express Foxp3 to achieve and maintain a T regulatory phenotype. Natural killer (NK) cells can be lymphocytes that are part of cell-mediated immunity and act during the innate immune response. NK cells do not require prior activation in order to perform their cytotoxic effect on target cells. Cytotoxic T cells (CTL or killer T cells) are a subset of T lymphocytes capable of inducing the death of infected somatic or tumor cells.

The immunoresponsive cells of the presently disclosed subject matter can express an extracellular antigen-binding domain (e.g., an scFV, a Fab that is optionally crosslinked, or a F(ab)₂) that specifically binds to FcRL5 (e.g., human FcRL5), for the treatment of multiple myeloma. Such immunoresponsive cells can be administered to a subject (e.g., a human subject) in need thereof for the treatment of multiple myeloma. In certain embodiments, the immunoresponsive cell is a T cell. The T cell can be a CD4⁺ T cell or a CD8⁺ T cell. In certain embodiments, the T cell is a CD4⁺ T cell. In another embodiment, the T cell is a CD8⁺ T cell.

A presently disclosed immunoresponsive cell can be further transduced with at least one co-stimulatory ligand, such that the immunoresponsive cell co-expresses or is induced to co-express the FcRL5-specific CAR and the at least one co-stimulatory ligand. The interaction between the FcRL5-specific CAR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the immunoresponsive cell is transduced with one co-stimulatory ligand that is 4-1BBL. In certain embodiments, the immunoresponsive cell is transduced with two co-stimulatory ligands that are 4-1BBL and CD80. CARs transduced with at least one co-stimulatory ligand are described in U.S. Pat. No. 8,389,282, which is incorporated by reference in its entirety.

Furthermore, a presently disclosed immunoresponsive cell can be further transduced with at least one cytokine, such that the immunoresponsive cell secretes the at least one cytokine as well as expresses the FcRL5-specific CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The FcRL5-specific or FcRL5-targeted human lymphocytes that can be used in peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, a presently disclosed immunoresponsive cell (e.g., T cell) expresses from about 1 to about 4, from about 2 to about 4, from about 3 to about 4, from about 1 to about 2, from about 1 to about 3, or from about 2 to about 3 vector copy numbers/cell of a presently disclosed FcRL5-specific CAR.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Preferably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

### IV. Vectors

Genetic modification of immunoresponsive cells (e.g., T cells, CTL cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the FcRL5-specific CAR can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide FcRL5-specific CAR expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a co-stimulatory ligand (e.g., 4-1BBL and IL-12) in an immunoresponsive cell. Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263 267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

In certain non-limiting embodiments, the vector expressing a presently disclosed FcRL5-targeted CAR is a retroviral vector, e.g., a 293galv9 retroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1 a enhancer/promoter/intron structure). For example, if desired, enhancers known to preferentially direct gene expression in specific cell types can be used to direct the expression of a nucleic acid. The enhancers used can include, without limitation, those that are characterized as tissue—or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### V. Polypeptides and Analogs and Polynucleotides

Also included in the presently disclosed subject matter are extracellular antigen-binding domains that specifically binds to FcRL5 (e.g., human FcRL5) (e.g., an scFv, such as an scFv derived from antibodies F56 and F119, a Fab, or a (Fab)₂), CD3ζ, CD8, CD28, etc. polypeptides or fragments thereof, and polynucleotides encoding thereof that are modified in ways that enhance their anti-tumor activity when expressed in an immunoresponsive cell. In certain embodiments, the presently disclosed subject matter further provides extracellular antigen-binding domains that specifically binds to domain 9 FcRL5 (e.g., domain 7, domain 8 or domain 9 of human FcRL5) (e.g., an scFv, a Fab, or a (Fab)₂), CD3ζ, CD8, CD28, etc. polypeptides or fragments thereof, and polynucleotides encoding thereof that are modified in ways that enhance their anti-tumor activity when expressed in an immunoresponsive cell.

The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity with all or part of a naturally-occurring amino, acid sequence of the presently disclosed subject matter. The length of sequence comparison is at least 5, 10, 15, 20, 25, 50, 75, 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least 5, 10, 13, or 15 amino acids. In certain embodiments, a fragment is at least 20 contiguous amino acids, at least 30 contiguous amino acids, or at least 50 contiguous amino acids. In certain embodiments, a fragment is at least 60 to 80, 100, 200, 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular antigen-binding domain that specifically binds to FcRL5 (e.g., an scFV (e.g., an scFv derived from antibodies F56 and F 119), a Fab, Fab′ or a (Fab′)₂), CD3ζ, CD8, CD28) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotids of the presently disclosed subject matter, including, but not limited to, OPTIMUMGENE™, Encor optimization, and Blue Heron.

### VI. Administration

FcRL5-specific CARs and immunoresponsive cells expressing thereof of the presently disclosed subject matter can be provided systemically or directly to a subject for treating or preventing a neoplasia. In certain embodiments, the FcRL5-specific CARs and immunoresponsive cells expressing thereof are directly injected into an organ of interest (e.g., an organ affected by a neoplasia). Alternatively or additionally, the FcRL5-specific CARs and immunoresponsive cells expressing thereof are provided indirectly to the organ of interest, for example, by administration into the circulatory system (e.g., the tumor vasculature). Expansion and differentiation agents can be provided prior to, during or after administration of cells and compositions to increase production of T cells in vitro or in vivo.

FcRL5-specific CARs and immunoresponsive cells expressing thereof of the presently disclosed subject matter can be administered in any physiologically acceptable vehicle, normally intravascularly, although they may also be introduced into bone or other convenient site where the cells may find an appropriate site for regeneration and differentiation (e.g., thymus). Usually, at least 1×10⁵ cells can be administered, eventually reaching 1×10¹⁰ or more. A cell population comprising immunoresponsive cells expressing a FcRL5-specific CAR can comprise a purified population of cells. Those skilled in the art can readily determine the percentage of immunoresponsive cells in a cell population using various well-known methods, such as fluorescence activated cell sorting (FACS). The ranges of purity in cell populations comprising genetically modified immunoresponsive cells expressing a FcRL5-specific CAR can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The immunoresponsive cells can be introduced by injection, catheter, or the like. If desired, factors can also be included, including, but not limited to, interleukins, e.g. IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

Compositions of the presently disclosed subject matter comprise pharmaceutical compositions comprising immunoresponsive cells expressing a FcRL5-specific CAR and a pharmaceutically acceptable carrier. Administration can be autologous or non-autologous. For example, immunoresponsive cells expressing a FcRL5-specific CAR and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising immunoresponsive cells expressing a FcRL5-specific CAR), it can be formulated in a unit dosage injectable form (solution, suspension, emulsion).

### VII. Formulations

Immunoresponsive cells expressing a generally FcRL5-specific CAR and compositions comprising thereof of the presently disclosed subject matter can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions comprising immunoresponsive cells expressing a generally FcRL5-specific CAR of the presently disclosed subject matter in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the present invention, however, any vehicle, diluent, or additive used would have to be compatible with the immunoresponsive cells expressing a generally FcRL5-specific CAR of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the immunoresponsive cells as describe in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the immunoresponsive cells of the presently disclosed subject matter is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ immunoresponsive cells of the presently disclosed subject matter are administered to a subject. More effective cells may be administered in even smaller numbers. In certain embodiments, at least about 1×10⁸, about 2×10⁸, about 3×10⁸, about 4×10⁸, and about 5×10⁸ immunoresponsive cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### VIII. Methods of Treatment

Tumor Microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including IL-10 and TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

Challenges in tumor immunology. Effective tumor immunity requires recognition of tumor antigens and unopposed tumor elimination by immune effector cells. Tumor antigens must contain peptide epitopes that are presented by the tumor and can be recognized by specific cytotoxic T lymphocytes (CTLs). The primed CTLs must expand to a sufficient number and migrate to tumor sites, wherein they mature into effectors to perform their functions, which are enhanced by helper T cells and dampened by Tregs and inhibitory macrophages.

Targeted T cell therapy with engineered T lymphocytes. T cell engineering is a groundbreaking strategy to potentially resolve many previously observed shortcomings of earlier immunotherapeutic approaches. Within the past year, researchers have reported dramatic complete remissions in relapsed (Brentjens et al., Blood 118, 4817-4828 (2011) and Brentjens et al., Science translational medicine 5, 177ra138 (2013)), chemorefractory leukemia and metastatic melanoma (Hunder et al., N. Engl.J.Med. 358, 2698-2703 (2008); Rosenberg et al., Nat. Rev. Cancer 8, 299-308 (2008); and Dudley et al., J Clin Oncol 26, 5233-5239 (2008)), obtained with autologous peripheral blood T cells targeted to a defined antigen (CD19 and NY-ESO-1, respectively).

Rationale for a genetic approach: Cell engineering can be used to redirect T cells toward tumor antigens and to enhance T cell function. One impetus for genetic T cell modification is the potential to enhance T cell survival and expansion and to offset T cell death, anergy, and immune suppression. The genetic targeting of T cells can also be refined to prevent undesired destruction of normal tissues.

Chimeric antigen receptors (CARs): Tumor-specific T cells can be generated by the transfer of genes that encode CARs (Brentjens et al., Clin.Cancer Res. 13, 5426-5435 (2007); Gade et al., Cancer Res. 65, 9080-9088 (2005); Maher et al., Nat.Biotechnol. 20, 70-75 (2002); Kershaw et al., J Immunol 173, 2143-2150 (2004); Sadelain et al., Curr Opin Immunol (2009); and Hollyman et al., J Immunother 32, 169-180 (2009)). Second-generation CARs comprise a tumor antigen-binding domain fused to an intracellular signaling domain capable of activating T cells and a co-stimulatory domain designed to augment T cell potency and persistence (Sadelain et al., Cancer discovery 3, 388-398 (2013)). CAR design can therefore reconcile antigen recognition with signal transduction, two functions that are physiologically borne by two separate complexes, the TCR heterodimer and the CD3 complex. The CAR's extracellular antigen-binding domain is usually derived from a murine monoclonal antibody (mAb) or from receptors or their ligands. Antigen recognition is therefore not MHC-restricted (Riviere et al., Curr Hematol Rep 3, 290-297 (2004); and Stephan et al., Nat. Med. 13, 1440-1449 (2007)) and is therefore applicable to any patient expressing the target antigen, using the same CAR. Antigen binding by the CARs triggers phosphorylation of immunoreceptor tyrosine-based activation motifs (ITAMs) in the intracellular domain, initiating a signaling cascade required for cytolysis induction, cytokine secretion, and proliferation. Because MHC restriction of antigen recognition is bypassed, the function of CAR-targeted T cells is not affected by HLA downregulation or defects in the antigen-processing machinery.

T cell requirements for expansion and survival: Proliferation of tumor-specific T cells is needed ex vivo and is arguably desirable in vivo. T cell proliferation must be accompanied by T cell survival to permit absolute T cell expansion and persistence. To proliferate in response to antigen, T cells must receive two signals. One is provided by TCR recognition of antigenic peptide/MHC complexes displayed on the surface of antigen-presenting cells (APCs) (Sadelain et al., Curr Opin Immunol (2009)). The other is provided by a T cell co-stimulatory receptor, such as the CD28 or 4-1BB receptors. Whereas the cytolytic activity of T cells does not require concomitant co-stimulation, there is a critical need for the provision of co-stimulatory signals to sustain the antitumor functions of adoptively transferred T cells, as previously demonstrated (Maher et al., Nat.Biotechnol. 20, 70-75 (2002); Sadelain et al., Cancer discovery 3, 388-398 (2013); Krause et al., J Exp Med 188, 619-626 (1998);Gong et al., Neoplasia. 1, 123-127 (1999); and Lyddane et al., J.Immunol. 176, 3306-3310 (2006)).

Immune monitoring: Lymphocytes are multifunctional “drugs” that exhibit dynamically evolving effects after infusion. Upon antigen encounter, tumor-specific T cells activate and/or release a variety of proteins that can trigger tumor killing, T cell proliferation, and recruitment or immunomodulation of other immune cells. Thus, measuring which proteins are secreted from which cells, in what quantity, and at what time point yields profound insights into why a particular patient is or is not responding and provides critical feedback for designing more-effective trials. These assay systems will permit direct and meaningful comparisons of clinical approaches and thus help design rational, next-generation therapeutic strategies.

For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific T cells, cell doses in the range of about 10⁶ to about 10¹⁰ (e.g., about 10⁹) are typically infused. Upon administration of the immunoresponsive cells into the subject and subsequent differentiation, the immunoresponsive cells are induced that are specifically directed against one specific antigen (e.g., FcRL5). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and the compositions comprising thereof are intravenously administered to the subject in need.

The presently disclosed subject matter provides various methods of using the immunoresponsive cells (e.g., T cells) expressing a FcRL5-specific CAR. For example, the presently disclosed subject matter provides methods of reducing tumor burden in a subject. In one non-limiting example, the method of reducing tumor burden comprises administering an effective amount of the presently disclosed immunoresponsive cell to the subject, thereby inducing tumor cell death in the subject. The presently disclosed immunoresponsive cell can reduce the number of tumor cells, reduce tumor size, and/or eradicate the tumor in the subject. Non-limiting examples of suitable tumors include multiple myeloma, Non-Hodgkin Lymphoma (especially Mantle Cell), Hodgkin Lymphoma, Chronic Lymphocytic Leukemia (CLL), Acute lymphocytic leukemia (ALL), Hairy Cell Leukemia, Burketts Lymphoma, and Waldenstrom's Macroglobulinemia.

The presently disclosed subject matter also provides methods of increasing or lengthening survival of a subject having a neoplasia. In one non-limiting example, the method of increasing or lengthening survival of a subject having neoplasia comprises administering an effective amount of the presently disclosed immunoresponsive cell to the subject, thereby increasing or lengthening survival of the subject. The method can reduce or eradicate tumor burden in the subject. The presently disclosed subject matter further provides methods for treating or preventing a neoplasia in a subject, comprising administering the presently disclosed immunoresponsive cell to the subject.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

Cancers whose growth may be inhibited using the immunoresponsive cells of the presently disclosed subject matter comprise cancers typically responsive to immunotherapy. Non-limiting examples of cancers for treatment include multiple myeloma, Non-Hodgkin Lymphoma (especially Mantle Cell), Hodgkin Lymphoma, Chronic Lymphocytic Leukemia (CLL), Acute lymphocytic leukemia (ALL), Hairy Cell Leukemia, Burketts Lymphoma, and Waldenstrom's Macroglobulinemia. In certain embodiments, the cancer is multiple myeloma.

Additionally, the presently disclosed subject matter provides methods of increasing immune-activating cytokine production in response to a cancer cell in a subject. In one non-limiting example, the method comprises administering the presently disclosed immunoresponsive cell to the subject. The immune-activating cytokine can be granulocyte macrophage colony stimulating factor (GM-CSF), IFN-α, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the immunoresponsive cells including an FcRL5-specific CAR of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor (e.g., multiple myeloma). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., multiple myeloma).

A second group of suitable subjects is known in the art as the “adjuvant group.” These are individuals who have had a history of neoplasia (e.g., multiple myeloma), but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor (e.g., multiple myeloma) has invaded neighboring tissues, or who show involvement of lymph nodes. Another group has a genetic predisposition to neoplasia (e.g., multiple myeloma) but has not yet evidenced clinical signs of neoplasia (e.g., multiple myeloma). For instance, women testing positive for a genetic mutation associated with breast cancer, but still of childbearing age, can wish to receive one or more of the antigen-binding fragments described herein in treatment prophylactically to prevent the occurrence of neoplasia until it is suitable to perform preventive surgery.

The subjects can have an advanced form of disease (e.g., multiple myeloma), in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the FcRL5-specific CAR-expressing immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the 3′ terminus of the intracellular domain of the FcRL5-specific CAR. The suicide gene can be included within the vector comprising nucleic acids encoding the presently disclosed FcRL5-specific CARs. In this way, administration of a prodrug designed to activate the suicide gene (e.g., a prodrug (e.g., AP1903 that can activates iCasp-9) during malignant T-cell transformation (e.g., GVHD) triggers apoptosis in the suicide gene-activated CAR-expressing T cells.

### IX. Kits

The presently disclosed subject matter provides kits for the treatment or prevention of a neoplasia (e.g., multiple myeloma). In certain embodiments, the kit comprises a therapeutic or prophylactic composition containing an effective amount of an immunoresponsive cell comprising a FcRL5-specific CAR in unit dosage form. In particular embodiments, the cells further expresses at least one co-stimulatory ligand. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the immunoresponsive cell is provided together with instructions for administering the cell to a subject having or at risk of developing a neoplasia (e.g., multiple myeloma). The instructions will generally include information about the use of the composition for the treatment or prevention of a neoplasia (e.g., multiple myeloma). In other embodiments, the instructions include at least one of the following: description of the therapeutic agent; dosage schedule and administration for treatment or prevention of a neoplasia (e.g., multiple myeloma) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### X. Exemplary extracellular antigen-binding domains (e.g., scFvs)

TABLE 1
ET200-001

DNA Sequence
(light chain variable region scFv linker heavy chain variable region His tag + HA tag)
Cagtctgtgttgacgcagccaccctcagcgtctgggacccccgggcagagggtcaccatctcttgttctggaagcagctccaacatcggaagta
atactgtaaactggtaccagcagctcccaggaacggcccccaaactcctcatctatagtaataatcagcggccctcaggggtccctgaccgattct
ctggctccaagtctggcacctcagcctccctggccatcagtgggctccagtctgaggatgaggctgattattactgtgcagcatgggatgacagc
ctgaatggttatgtcttcggaactgggaccaaggtcaccgtcctaggt [SEQ ID NO: 1]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccctcacctgcgctgtgtatggtgggtccttcagt
ggttactactggagctggatccgccagcccccagggaaggggctggagtggattggggaaatcaatcatagtggaagcaccaactaca
acccgtccctcaagagtcgagtcaccatatcagtagacacgtccaagaaccagttctccctgaagctgagctctgtgaccgccgcggac
acggccgtgtattactgtgcgcgcgaaggtccgtacgacggtttcgattcttggggtcaaggtactctggtgaccgtctcctca [SEQ
ID NO: 2]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYVFGTGTKVTVLG [SEQ ID NO: 3]
SRGGGGSGGGGSGGGGSLEMA [SEQ ID NO: 307]
QVQLQQWGAGLLKPSETLSLTCAVYGGSFSGYYWSWIRQPPGKGLEWIGEINHSGST
NYNPSLKSRVTISVDTSKNQFSLKLSSVTAADTAVYYCAREGPYDGFDSWGQGTLVTV
SS [SEQ ID NO: 4]
TSGQAGQHHHHHHGAYPYDVPDYAS [SEQ ID NO: 308]

TABLE 2
ET200-002

DNA Sequence
(light chain variable region scFv linker heavy chain variable region His tag + HA tag)
Aattttatgctgactcagccccactctgtgtcggagtctccggggaagacggtaaccatctcctgcacccgcagcagtggcagc
attgccagcaactatgtgcagtggtaccagcagcgcccgggcagtgcccccaccactgtgatctatgaggataaccaaagacc
ctctggggtccctgatcggttctctggctccatcgacagctcctccaactctgcctccctcaccatctctggactgaagactgagg
acgaggctgactactactgtcagtcttatgatagcagcaattctgtggtattcggcggagggaccaagctgaccgtcctaggt
[SEQ ID No. 5]
tctagaggtggtggtggtagcggcggcggcggctctggtggtggtggatccctcgagatggcc [SEQ ID NO: 305]
caggtccagctggtacagtctggcactgaggtgaagaagcctggggcctcagtgagggtcgcctgcaaggcttctggtt
acccctttaacaaatatgacatcaactgggtgcgacaggcccctggacaagggcttgagtggatgggaggcatcatccc
tatctttcgtacaacaaactacgcacagaagttccagggcagagtcacgattaccgcggacgaatccacgagcacagcc
tacatggagctgagcagcctgagatctgaggacacggccgtatattactgtgcgcgcgaatggttctactgggatatctg
gggtcaaggtactctggtgaccgtctcctca [SEQ ID NO: 6]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSSNSVVFGGGTKLTVLG
[SEQ ID NO: 7]
SRGGGGSGGGGSGGGGSLEMA [SEQ ID NO: 307]
QVQLVQSGTEVKKPGASVRVACKASGYPFNKYDINWVRQAPGQGLEWMG
GIIPIFRTTNYAQKFQGRVTITADESTSTAYMELSSLRSEDTAVYYCAREWFY
WDIWGQGTLVTVSS [SEQ ID NO: 8]
TSGQAGQHHHHHHGAYPYDVPDYAS [SEQ ID NO: 308]

TABLE 3
ET200-003

DNA Sequence
(light chain variable region scFv linker heavy chain variable region His tag + HA tag)
cagtctgtgttgactcagccaccctcagtgtccgtgtccccaggacagacagccagcatctcctgctctggaaataaattgggga
ctaagtatgtttactggtatcagaagaggccaggccagtcccctgtgttggtcatgtatgaagataatcagcggccctcagggatc
ccggagcggttctctggctccaactctgggaacacagccactctgaccatcagagggacccagactgtggatgaggctgactat
tactgtcaggcgtgggactccgacactttcgtggtcttcggcggagggaccaaggtcaccgtcctaggt [SEQ ID NO: 9]
tctagaggtggtggtggtagcggcggcggcggctctggtggtggtggatccctcgagatggcc [SEQ ID NO: 305]
gaggtgcagctggtggagaccgggggaggcgtggtccagcctgggaggtccctgagactctcctgtgcagcctctggat
tcaccttcagtagttatggcatgcactgggtccgccaggctccaggcaaggggctggagtgggtggcagttatatcacat
gatggaagtaataaatactacgcagactccgtgaagggccgattcaccatctccagagacaattccaaggacacgctgt
atctgcaaatgaacagcctgagaggtgaggacacggccgtatattactgtgcgcgctctaaccagtggtctggttacttct
ctttcgattactggggtcaaggtactctggtgaccgtctcctca [SEQ ID NO: 10]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSVSVSPGQTASISCSGNKLGTKYVYWYQKRPGQSPVLVMYEDNQRP
SGIPERFSGSNSGNTATLTIRGTQTVDEADYYCQAWDSDTFVVFGGGTKVTVLG
[SEQ ID NO: 11]
SRGGGGSGGGGSGGGGSLEMA [SEQ ID NO: 307]
EVQLVETGGGVVQPGRSLRLSCAASGFTFSSYGMHWVRQAPGKGLEWVAV
ISHDGSNKYYADSVKGRFTISRDNSKDTLYLQMNSLRGEDTAVYYCARSNQ
WSGYFSFDYWGQGTLVTVSS [SEQ ID NO: 12]
TSGQAGQHHHHHHGAYPYDVPDYAS [SEQ ID NO: 308]

TABLE 4
ET200-006

DNA Sequence
(light chain variable region scFv linker heavy chain variable region His tag + HA tag)
tcctatgtgctgactcagccaccctcagtgtcagtggccccaggaaagacggccaggattacctgtgggggaaacaacattgga
agtaaaagtgtgcactggtaccagcagaagccaggccaggcccctgtggtggtcatccattatgatagcgaccggccctcagg
gatccctgagcgattctctggctccaactctgggaacacggccaccctgaccatcagcagggtcgaagccggggatgaggcc
gactattactgtcaggtgtgggatagtagtagtgatcatccttatgtcttcggaactgggaccaaggtcaccgtcctaggt [SEQ
ID NO: 13]
tctagaggtggtggtggtagcggcggcggcggctctggtggtggtggatccctcgagatggcc [SEQ ID NO: 305]
gaggtgcagctggtgcagtctggagctgaggtgaagaagcctggggcctcagtgaaggtctcctgcaaggcttctggtt
acacctttaccacctatggtatcagctgggtgcgacaggcccctggacaagggcttgagtggatgggatggatcaacac
ttacaatggtcacacaaactatgcacagaagctccagggcagagccacaatgaccgcagacacatccacgaacacag
cctacatggagctgaggagcctgagatctgacgacactgccgtgtattactgtgcgcgcgttatctacggttctggtgatt
actggggtcaaggtactctggtgaccgtctcctca [SEQ ID NO: 14]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SYVLTQPPSVSVAPGKTARITCGGNNIGSKSVHWYQQKPGQAPVVVIHYDSDRP
SGIPERFSGSNSGNTATLTISRVEAGDEADYYCQVWDSSSDHPYVFGTGTKVTVLG
[SEQ ID NO: 15]
SRGGGGSGGGGSGGGGSLEMA [SEQ ID NO: 307]
EVQLVQSGAEVKKPGASVKVSCKASGYTFTTYGISWVRQAPGQGLEWMG
WINTYNGHTNYAQKLQGRATMTADTSTNTAYMELRSLRSDDTAVYYCARV
IYGSGDYWGQGTLVTVSS [SEQ ID NO: 16]
TSGQAGQHHHHHHGAYPYDVPDYAS [SEQ ID NO: 308]

TABLE 5
ET200-007

DNA Sequence
(light chain variable region scFv linker heavy chain variable region His tag + HA tag)
tcctatgtgctgactcagccactctcagtgtcagtggccccaggaaagacggccaggattacctgtgggggaaacaacattgga
agtaaaactgtgcactggtaccagcagaagccaggccaggcccctgtgctggtcatctattatgatagcgaccggccctcaggg
atccctgagcgattctctggctccaactctgggaacacggccaccctgaccatcagcagggtcgaagccggggatgaggccg
actattactgtcaggtgtgggatagtagtagtgatcatcgggtgttcggcggagggaccaagctgaccgtcctaggt [SEQ
ID NO: 17]
tctagaggtggtggtggtagcggcggcggcggctctggtggtggtggatccctcgagatggcc [SEQ ID NO: 305]
caggtgcagctgcaggagtcgggcccaggactggtgaagccttcggagaccctgtccctcacctgcaatgtctctggtta
ctccatcagcagtggttacttttggggctggatccggcagcccccagggaaggggctggagtggattgggagtatctatc
atagtaggagcacctactacaacccgtccctcaagagtcgagtcaccatatcagtagacacgtccaagaaccagttctc
cctgaagctgaactctgtgaccgccgcagacacggccgtgtattactgtgcgcgcggttacggttacttcgattactgggg
tcaaggtactctggtgaccgtctcctca [SEQ ID NO: 18]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SYVLTQPLSVSVAPGKTARITCGGNNIGSKTVHWYQQKPGQAPVLVIYYDSDRP
SGIPERFSGSNSGNTATLTISRVEAGDEADYYCQVWDSSSDHRVFGGGTKLTVLG
[SEQ ID NO: 19]
SRGGGGSGGGGSGGGGSLEMA [SEQ ID NO: 307]
QVQLQESGPGLVKPSETLSLTCNVSGYSISSGYFWGWIRQPPGKGLEWIGSI
YHSRSTYYNPSLKSRVTISVDTSKNQFSLKLNSVTAADTAVYYCARGYGYFD
YWGQGTLVTVSS [SEQ ID NO: 20]
TSGQAGQHHHHHHGAYPYDVPDYAS [SEQ ID NO: 308]

TABLE 6
ET200-008

DNA Sequence
(light chain variable region scFv linker heavy chain variable region His tag + HA tag)
caatctgccctgactcagcctgcctccgtgtctgggtctcctggacagtcgatcaccatctcctgcactggaaccagcagtgacgt
tggtggttataactatgtctcctggtaccaacaacacccaggcaaagcccccaaactcatgatttatgatgtcagtaatcggccctc
aggggtttctaatcgcttctctggctccaagtctggcaacacggcctccctgaccatctctgggctccaggctgaggacgaggct
gattattactgcagctcatatacaagcagcagcacttcgaaggtgttcggcggagggaccaagctgaccgtcctaggt [SEQ
ID NO: 21]
tctagaggtggtggtggtagcggcggcggcggctctggtggtggtggatccctcgagatggcc [SEQ ID NO: 305]
gaggtgcagctggtggagtctgggggaggtgtggtacggcctggggggtccctgagactctcctgtgcagcctctggatt
cacctttggtgattatggcatgagctgggtccgccaagctccagggaaggggctggagtgggtctctggtattaattgga
atggtggtagcacaggttatgcagactctgtgaagggccgattcaccatctccagagacaacgccaagaactccctgtat
ctgcaaatgaacagtctgagagccgaggacacggccgtatattactgtgcgcgctctaaatacaacttccatgtttactac
gattactggggtcaaggtactctggtgaccgtctcctca [SEQ ID NO: 22]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSALTQPASVSGSPGQSITISCTGTSSDVGGYNYVSWYQQHPGKAPKLMIYDVSN
RPSGVSNRFSGSKSGNTASLTISGLQAEDEADYYCSSYTSSSTSKVFGGGTKLTVLG
[SEQ ID NO: 23]
SRGGGGSGGGGSGGGGSLEMA [SEQ ID NO: 307]
EVQLVESGGGVVRPGGSLRLSCAASGFTFGDYGMSWVRQAPGKGLEWVSG
INWNGGSTGYADSVKGRFTISRDNAKNSLYLQMNSLRAEDTAVYYCARSKY
NFHVYYDYWGQGTLVTVSS [SEQ ID NO: 24]
TSGQAGQHHHHHHGAYPYDVPDYAS [SEQ ID NO: 308]

TABLE 7
ET200-009

DNA Sequence
(light chain variable region scFv linker heavy chain variable region His tag + HA tag)
cagtctgtgttgacgcagccaccctcagcgtctgggacccccgggcagacagtcaccatctcttgttctggaagcaactccaac
atcggaagtaattatgtatactggtaccagcagctcccaggaacggcccccaaactcctcatctataggaataatcagcggccct
caggggtccctgaccgattctcaggctccaagtctggcacctcagcctccctggccatcagtgggctccgctccgaggatgag
gctgattattactgtgcagcatgggatgacagcctgagtgcttatgtcttcggaactgggaccaaggtcaccgtcctaggt
[SEQ ID NO: 25]
tctagaggtggtggtggtagcggcggcggcggctctggtggtggtggatccctcgagatggcc [SEQ ID NO: 305]
caggtgcagctggtgcagtctggagctgaggtgaagaagcctggggcctcagtgaaggtctcctgcaaggcttctggtt
acacctttaccagctatggtatcagctgggtgcgacaggcccctggacaagggcttgagtggatgggatggatcagcgc
ttacaatggtaacacaaactatgcacagaagctccagggcagagtcaccatgaccacagacacatccacgagcacagc
ctacatggagctgaggagcctgagatctgacgacactgccgtgtattactgtgcgcgctcttctggtaacatggtttcttgg
aaagatatgtggggtcaaggtactctggtgaccgtctcctca [SEQ ID NO: 26]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSASGTPGQTVTISCSGSNSNIGSNYVYWYQQLPGTAPKLLIYRNNQR
PSGVPDRFSGSKSGTSASLAISGLRSEDEADYYCAAWDDSLSAYVFGTGTKVTVLG
[SEQ ID NO: 27]
SRGGGGSGGGGSGGGGSLEMA [SEQ ID NO: 307]
QVQLVQSGAEVKKPGASVKVSCKASGYTFTSYGISWVRQAPGQGLEWMG
WISAYNGNTNYAQKLQGRVTMTTDTSTSTAYMELRSLRSDDTAVYYCARSS
GNMVSWKDMWGQGTLVTVSS [SEQ ID NO: 28]
TSGQAGQHHHHHHGAYPYDVPDYAS [SEQ ID NO: 308]

TABLE 8
ET200-010

DNA Sequence
(light chain variable region scFv linker heavy chain variable region His tag + HA tag)
caatctgccctgactcagcctgcctccgtgtctgggtctcctggacagtcgatcaccatctcctgcactggaaccagcagtgacgt
tggtggttataactctgtctcctggtaccaacaacacccaggcaaagcccccagactcatgatttatgatgtcagtaatcggccctc
aggggtttctaatcgcttctctggctccaagtctggcaacacggcctccctgaccatctctgggctccaggctgaggacgaggct
gattattactgcagctcatatacaagcagcagcacccctttagtcttcggaactgggaccaaggtcaccgtcctaggt [SEQ
ID NO: 29]
tctagaggtggtggtggtagcggcggcggcggctctggtggtggtggatccctcgagatggcc [SEQ ID NO: 305]
caggtgcagctggtgcagtctggggctgaggtgaagaagcctggggcctcagtgaaggtctcctgcaaggcttctggtt
acacctttaccagctatggtatcagctgggtgcgacaggcccctggacaagggcttgagtggatgggatggatcagcgc
ttacaatggtaacacaaactatgcacagaagctccagggcagagtcaccatgaccacagacacatccacgagcacagc
ctacatggagctgaggagcctgagatctgacgacacggccgtgtattactgtgcgcgcggtgctgttgcttaccatgattg
gggtcaaggtactctggtgaccgtctcctca [SEQ ID NO: 30]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSALTQPASVSGSPGQSITISCTGTSSDVGGYNSVSWYQQHPGKAPRLMIYDVSN
RPSGVSNRFSGSKSGNTASLTISGLQAEDEADYYCSSYTSSSTPLVFGTGTKVTVLG
[SEQ ID NO: 31]
SRGGGGSGGGGSGGGGSLEMA [SEQ ID NO: 307]
QVQLVQSGAEVKKPGASVKVSCKASGYTFTSYGISWVRQAPGQGLEWMG
WISAYNGNTNYAQKLQGRVTMTTDTSTSTAYMELRSLRSDDTAVYYCARG
AVAYHDWGQGTLVTVSS [SEQ ID NO: 32]
TSGQAGQHHHHHHGAYPYDVPDYAS [SEQ ID NO: 308]

TABLE 9
ET200-011

DNA Sequence
(light chain variable region scFv linker heavy chain variable region His tag + HA tag)
cagtctgtcgtgacgcagccgccctcagtgtctgcggccccaggacagagggtcaccatctcctgctctggaagcagctccaa
catttcgatttatgatgtatcctggtatcagcagctcccaggaacagcccccaaactcctcatttatggcaataataagcgaccctc
ggggattgctgaccgattctctggctccacgtctggcacgtcagccaccctgggcatcaccggactccagactggggacgagg
ccgattattactgcggaacatgggatgacagtctgagtgggggggtgttcggcggagggaccaagctgaccgtcctaggt
[SEQ ID NO: 33]
tctagaggtggtggtggtagcggcggcggcggctctggtggtggtggatccctcgagatggcc [SEQ ID NO: 305]
cagatgcagctggtgcaatctggggctgaggtgaagaagcctgggtcctcggtgaaggtctcctgcgaggcttctggag
gcaccctcagcagctatgctatcaactgggtgcgacaggcccctggacaagggcttgagtggatgggagggatcatccc
tatgtttggtacagcacactacgcacagaagttccagggcagagtcacgattaccgcggacgaatccacgaaaacagc
ctacatggagctgagcagcctgagatctgaggacactgccgtgtattactgtgcgcgcggtgttcattacgcttctttcgat
cattggggtcaaggtactctggtgaccgtctcctca [SEQ ID NO: 34]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVVTQPPSVSAAPGQRVTISCSGSSSNISIYDVSWYQQLPGTAPKLLIYGNNKRP
SGIADRFSGSTSGTSATLGITGLQTGDEADYYCGTWDDSLSGGVFGGGTKLTVLG
[SEQ ID NO: 35]
SRGGGGSGGGGSGGGGSLEMA [SEQ ID NO: 307]
QMQLVQSGAEVKKPGSSVKVSCEASGGTLSSYAINWVRQAPGQGLEWMGG
IIPMFGTAHYAQKFQGRVTITADESTKTAYMELSSLRSEDTAVYYCARGVHY
ASFDHWGQGTLVTVSS [SEQ ID NO: 36]
TSGQAGQHHHHHHGAYPYDVPDYAS [SEQ ID NO: 308]

TABLE 10
ET200-012

DNA Sequence
(light chain variable region scFv linker heavy chain variable region His tag + HA tag)
cagtctgtgttgacgcagccgccctcagtgtctgcggccgcaggacagaaggtcaccatctcctgctctggaagcgactccaac
attgggaataattatgtgtcctggtatcaacacctcccagggacagcccccaaactcctcatttatgacgttaaaaatcgaccctca
gggattcctgaccggttctccggctccaagtctggctcgtcagccaccctaggcatcgccggactccagcctggggacgaggc
cgattattactgcggaacatgggacagtcggctggatgcctatgtcttcggaactgggaccaaggtcaccgtcctaggt [SEQ
ID NO: 37]
tctagaggtggtggtggtagcggcggcggcggctctggtggtggtggatccctcgagatggcc [SEQ ID NO: 305]
cagatgcagctggtgcaatctggagctgaggtgaagaagcctggggcctcagtgaaggtctcctgcaagacttctggttt
cccctttaatatctttggaatcacctgggtgcgacaggcccctggacaagggcttgagtggatgggatggatcagcggtt
acaacggtaacacagactacccacagaagttccagggcagagtcaccatgtccacagacacatccacgagtacagcct
acatggagctgaggaacctgaaatctgacgacacggccgtgtattactgtgcgcgcggtgcttacggtggtatggatact
tggggtcaaggtactctggtgaccgtctcctca [SEQ ID NO: 38]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSVSAAAGQKVTISCSGSDSNIGNNYVSWYQHLPGTAPKLLIYDVKNR
PSGIPDRFSGSKSGSSATLGIAGLQPGDEADYYCGTWDSRLDAYVFGTGTKVTVLG
[SEQ ID NO: 39]
SRGGGGSGGGGSGGGGSLEMA [SEQ ID NO: 307]
QMQLVQSGAEVKKPGASVKVSCKTSGFPFNIFGITWVRQAPGQGLEWMGW
ISGYNGNTDYPQKFQGRVTMSTDTSTSTAYMELRNLKSDDTAVYYCARGAY
GGMDTWGQGTLVTVSS [SEQ ID NO: 40]
TSGQAGQHHHHHHGAYPYDVPDYAS [SEQ ID NO: 308]

TABLE 11
ET200-013

DNA Sequence
(light chain variable region scFv linker heavy chain variable region His tag + HA tag)
cagtctgtcgtgacgcagccgccctcagtgtctggggccccagggcagagggtcaccatctcctgcactgggagcacctccaa
catcggggcaggttatgatgtacactggtatcagcagcttccaggaacagcccccaaactcctcatctatactaacaactttcggc
cctcaggggtccctgaccgattctctgcctccaagtctggcacttcagcttccctggccatcactggtctccaggctgaggatgag
gctgattattactgcggaacatgggatagcagcctgagtgccgttgtgttcggcggagggaccaagctgaccgtcctaggt
[SEQ ID NO: 41]
tctagaggtggtggtggtagcggcggcggcggctctggtggtggtggatccctcgagatggcc [SEQ ID NO: 305]
gaggtgcagctggtggagtctggaactgaggtgaagaagcctggggcctcagtgaaagtctcctgcaaggcttctggtt
acatgtttaccagttatggtctcaactgggtgcgacaggcccctggacaagggcttgagtggatgggatggatcagcgct
aacaatggtaagacaaattatgctaagaaattccaggacagagtcaccatgaccagagacacttccacgagcacaggc
tacatggaactgaggagcctgagatctgacgacacggccgtatattactgtgcgcgccatatcggtggttcttacttcgat
cgttggggtcaaggtactctggtgaccgtctcctca [SEQ ID NO: 42]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVVTQPPSVSGAPGQRVTISCTGSTSNIGAGYDVHWYQQLPGTAPKLLIYTNNF
RPSGVPDRFSASKSGTSASLAITGLQAEDEADYYCGTWDSSLSAVVFGGGTKLT
VLG [SEQ ID NO: 43]
SRGGGGSGGGGSGGGGSLEMA [SEQ ID NO: 307]
EVQLVESGTEVKKPGASVKVSCKASGYMFTSYGLNWVRQAPGQGLEWMG
WISANNGKTNYAKKFQDRVTMTRDTSTSTGYMELRSLRSDDTAVYYCARHI
GGSYFDRWGQGTLVTVSS [SEQ ID NO: 44]
TSGQAGQHHHHHHGAYPYDVPDYAS [SEQ ID NO: 308]

TABLE 12
ET200-014

DNA Sequence
(light chain variable region scFv linker heavy chain variable region His tag + HA tag)
tcctatgtgctgactcagccaccctcagtgtcagtggccccaggaaagacggccaggattacctgtgggggaaacaacattgga
agtaaaagtgtgcactggtaccagcagaagccaggccaggcccctgtgctggtcatctattatgatagcgaccggccctcagg
gatccctgagcgattctctggctccaactctgggaacacggccaccctgaccatcagcagggtcgaagccggggatgaggcc
gactattactgtcaggtgtgggatagtagtagtgatcattatgtcttcggaactgggaccaaggtcaccgtcctaggt [SEQ ID
NO: 45]
tctagaggtggtggtggtagcggcggcggcggctctggtggtggtggatccctcgagatggcc [SEQ ID NO: 305]
gaggtgcagctggtggagactgggggaggcttggtacagcctggggggtccctgagactctcctgtgcagcctctggatt
cacctttagcagctatgccatgagctgggtccgccaggctccagggaaggggctggagtgggtctcagctattagtggta
gtgatggtagcacatactacgcagactccgtgaagggccggttcaccatctccagagacaattccaagaacacgctgta
tctgcaaatgaacagcctgagagacgaggacacggccgtatattactgtgcgcgctctcatgaagctaacctggttggtg
attggtggggtcaaggtactctggtgaccgtctcctca [SEQ ID NO: 46]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SYVLTQPPSVSVAPGKTARITCGGNNIGSKSVHWYQQKPGQAPVLVIYYDSDRPS
GIPERFSGSNSGNTATLTISRVEAGDEADYYCQVWDSSSDHYVFGTGTKVTVLG
[SEQ ID NO: 47]
SRGGGGSGGGGSGGGGSLEMA [SEQ ID NO: 307]
EVQLVETGGGLVQPGGSLRLSCAASGFTFSSYAMSWVRQAPGKGLEWVSAI
SGSDGSTYYADSVKGRFTISRDNSKNTLYLQMNSLRDEDTAVYYCARSHEAN
LVGDWWGQGTLVTVSS [SEQ ID NO: 48]
TSGQAGQHHHHHHGAYPYDVPDYAS [SEQ ID NO: 308]

TABLE 13
ET200-015

DNA Sequence
(light chain variable region scFv linker heavy chain variable region His tag + HA tag)
cagtctgtggtgactcagccaccctcagtgtcagtggccccaggaaagacggccaggattacctgtgggggaaacaacattgg
aagtaaaagtgtgcactggtaccagcagaagccaggccaggcccctgtgctggtcatctattatgatagcgaccggccctcagg
gatccctgagcgattctctggctccaactctgggaacacggccaccctgaccatcagcagggtcgaagccggggatgaggcc
gactattactgtcaggtgtgggatagtagtagtgatgtggtattcggcggagggaccaagctgaccgtcctaggt [SEQ ID
NO: 49]
tctagaggtggtggtggtagcggcggcggcggctctggtggtggtggatccctcgagatggcc [SEQ ID NO: 305]
gaggtccagctggtacagtctggagctgaggtgaagaagcctggggcctcagtgaaggtctcctgcaaggcttctggtt
acacctttaccagctacggtatcagctgggtgcgacaggcccctggacaagggcttgagtggatgggatggatcagcgc
ttacaatggtaacacaaactatgcacagaagctccagggcagagtcaccatgaccacagacacatccacgagcacagc
ctacatggagctgaggagcctgagatctgacgacacggccgtgtattactgtgcgcgctggggtggtttcggtgctgttg
atcattggggtcaaggtactctggtgaccgtctcctca [SEQ ID NO: 50]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVVTQPPSVSVAPGKTARITCGGNNIGSKSVHWYQQKPGQAPVLVIYYDSDRP
SGIPERFSGSNSGNTATLTISRVEAGDEADYYCQVWDSSSDVVFGGGTKLTVLG
[SEQ ID NO: 51]
SRGGGGSGGGGSGGGGSLEMA [SEQ ID NO: 307]
EVQLVQSGAEVKKPGASVKVSCKASGYTFTSYGISWVRQAPGQGLEWMGW
ISAYNGNTNYAQKLQGRVTMTTDTSTSTAYMELRSLRSDDTAVYYCARWG
GFGAVDHWGQGTLVTVSS [SEQ ID NO: 52]
TSGQAGQHHHHHHGAYPYDVPDYAS [SEQ ID NO: 308]

TABLE 14
ET200-016

DNA Sequence
(light chain variable region scFv linker heavy chain variable region His tag + HA tag)
tcttctgagctgactcaggaccctgctgtgtctgtggccttgggacagacagtcaagatcacgtgccaaggagacagcctcaca
gactaccatgcaacctggtaccagcagaagccaggacaggcccctgtcgctgtcatctatgctacaaacaaccggcccactgg
gatcccagaccgattctctggttccagttccggaaacacagcttctttgaccatcactggggctcaggcggaagatgaggctgac
tattactgtaattcccgggacagcggcacggacgaagtgttattcggcggagggaccaagctgaccgtcctaggt [SEQ ID
NO: 53]
tctagaggtggtggtggtagcggcggcggcggctctggtggtggtggatccctcgagatggcc [SEQ ID NO: 305]
gaggtgcagctggtggagactgggggaggcctggtcaagcctggggggtccctgagactctcctgtgcagcctctggat
tcaccttcagtagctatagcatgaactgggtccgccaggctccagggaaggggctggagtgggtctcatccattagtagt
agtagtagttacatatactacgcagactcagtgaagggccgattcaccatctccagagacaacgccaagaactcactgt
atctgcaaatgaacagcctgagagccgaggacacggccgtgtattactgtgcgcgcggtcagggttacgattactgggg
tcaaggtactctggtgaccgtctcctca [SEQ ID NO: 54]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SSELTQDPAVSVALGQTVKITCQGDSLTDYHATWYQQKPGQAPVAVIYATNNRP
TGIPDRFSGSSSGNTASLTITGAQAEDEADYYCNSRDSGTDEVLFGGGTKLTVLG
[SEQ ID NO: 55]
SRGGGGSGGGGSGGGGSLEMA [SEQ ID NO: 307]
EVQLVETGGGLVKPGGSLRLSCAASGFTFSSYSMNWVRQAPGKGLEWVSSI
SSSSSYIYYADSVKGRFTISRDNAKNSLYLQMNSLRAEDTAVYYCARGQGYD
YWGQGTLVTVSS [SEQ ID NO: 56]
TSGQAGQHHHHHHGAYPYDVPDYAS [SEQ ID NO: 308]

TABLE 15
ET200-017

DNA Sequence
(light chain variable region scFv linker heavy chain variable region His tag + HA tag)
tcctatgtgctgactcagccaccctcggtgtcagtggccccaggaaagacggccaggat
tacctgtgggggaaacaacattggaagtaaaagtgtgcactggtaccagcagaagccag
gccaggcccctgtgctggtcgtctatgatgatagcgaccggccctcagggatccctgag
cgattctctggctccaactctgggaacacggccaccctgagcatcagcagggtcgaagc
cggggatgaggccgactattactgtcaggtgtgggatagtagtagtgatcatactgtct
tcggaactgggaccaaggtcaccgtcctaggt [SEQ ID NO: 57]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccct
cacctgcgctgtctatggtgggtccttcagtggttactactggagctggatccgccagc
ccccagggaaggggctggagtggattggggaaatcaatcatagtggaagcaccaactac
aacccgtccctcaagagtcgagtcaccatatcagtagacacgtccaagaaccagttctc
cctgaagctgagctctgtgaccgccgcggacacggccgtgtattactgtgcgcgctact
acccgggtatggatatgtggggtcaaggtactctggtgaccgtctcctca [SEQ ID NO: 58]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
SYVLTQPPSVSVAPGKTARITCGGNNIGSKSVHWYQQKPGQAPVLVVYDDSDRPSGIPE
RFSGSNSGNTATLSISRVEAGDEADYYCQVWDSSSDHTVFGTGTKVTVLG [SEQ ID NO: 59]
SRGGGGSGGGGSGGGGSLEMA [SEQ ID NO: 307]
QVQLQQWGAGLLKPSETLSLTCAVYGGSFSGYYWSWIRQPPGKGLEWIGEINHSGSTNY
NPSLKSRVTISVDTSKNQFSLKLSSVTAADTAVYYCARYYPGMDMWGQGTLVTVSS
[SEQ ID NO: 60]
TSGQAGQHHHHHHGAYPYDVPDYAS [SEQ ID NO: 308]

TABLE 16
ET200-018

DNA Sequence
(light chain variable region scFv linker heavy chain variable region His tag + HA tag)
caggctgtgctgactcagccgccctcaacgtctgggacccccgggcagagggtcaccat
ctcttgttctggaagcagctccaacatcgggagaaatggtgtaaactggtaccagcagc
tcccaggagcggcccccaaagtcctcatctataatgataatcagcgaccctcaggggtc
cctgaccgagtctctggctcccagtctggctcctcaggcaccctggccatcgatgggct
tcggtctgaggatgaggctgattattactgtgcggcatgggatgacagcctgcatggtg
tggtattcggcggagggaccaagctgaccgtcctaggt [SEQ ID NO: 61]
tctagaggtggtggtggtagcggcggcggcggctctggtggtggtggatccctcgagatggcc [SEQ ID NO: 305]
caggtccagctggtacagtctggggctgaggtgaagaagcctggggcctcagtgaaggt
ctcctgcaaggtttccggatacaccctcaatgaattatccatgcactgggtgcgacagg
ctcctggaaaagggcttgagtggatgggaggttttgatcctgaagatggtgaaacaatc
tacgcacagaagttccagggcagagtcaccatgaccgaggacacatctacagacacagc
ctacatggagctgagcagcctgagatctgaggacactgccgtgtattactgtgcgcgcg
gtggttacggtgattcttggggtcaaggtactctggtgaccgtctcctca [SEQ ID NO: 62]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QAVLTQPPSTSGTPGQRVTISCSGSSSNIGRNGVNWYQQLPGAAPKVLIYNDNQRPSGV
PDRVSGSQSGSSGTLAIDGLRSEDEADYYCAAWDDSLHGVVFGGGTKLTVLG [SEQ ID NO: 63]
SRGGGGSGGGGSGGGGSLEMA [SEQ ID NO: 307]
QVQLVQSGAEVKKPGASVKVSCKVSGYTLNELSMHWVRQAPGKGLEWMGGFDPEDGETI
YAQKFQGRVTMTEDTSTDTAYMELSSLRSEDTAVYYCARGGYGDSWGQGTLVTVSS
[SEQ ID NO: 64]
TSGQAGQHHHHHHGAYPYDVPDYAS [SEQ ID NO: 308]

TABLE 17
ET200-019

DNA Sequence
(light chain variable region scFv linker heavy chain variable region His tag + HA tag)
aattttatgctgactcagccccactctgtgtcggagtctccggggaagacggtaaccat
ctcctgcacccgcagcagtggcagcattgccagcaactatgtgcagtggtaccagcagc
gcccgggcagtgcccccaccactgtgatctatgaggataaccaaagaccctctggggtc
cctgatcggttctctggctccatcgacagctcctccaactctgcctccctcaccatctc
tggactgaagactgaggacgaggctgactactactgtcagtcttatgatagcagcaatt
cttgggtgttcggcggagggaccaagctgaccgtcctaggt [SEQ ID NO: 65]
tctagaggtggtggtggtagcggcggcggcggctctggtggtggtggatccctcgagatggcc [SEQ ID NO: 305]
caggtgcagctggtgcaatctggggctgaggtgaagaggcctgggtcctcggtgaaggt
ctcctgcacggcttctggaggcaccttcagcagcgatgctatcagctgggtgcgacagg
cccctggacaagggcttgagtggatgggaggaatcatccctatgtttggtacagcaaac
tacgcacagaagttccagggcagagtcacgattaccgcggacgaatccacgagcacagc
ctacatggagctgagcagcctgagatctgaggacacggccgtgtattactgtgcgcgcg
aaggttactactacccgtctgcttacctgggttctgttctgaacgacatctcttctgtt
tacgatgaatggggtcaaggtactctggtgaccgtctcctca [SEQ ID NO: 66]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSESPGKTVTISCTRSSGSIASNYVQWYQQRPGSAPTTVIYEDNQRPSGV
PDRFSGSIDSSSNSASLTISGLKTEDEADYYCQSYDSSNSWVFGGGTKLTVLG [SEQ ID NO: 67]
SRGGGGSGGGGSGGGGSLEMA [SEQ ID NO: 307]
QVQLVQSGAEVKRPGSSVKVSCTASGGTFSSDAISWVRQAPGQGLEWMGGIIPMFGTAN
YAQKFQGRVTITADESTSTAYMELSSLRSEDTAVYYCAREGYYYPSAYLGSVLNDISSV
YDEWGQGTLVTVSS [SEQ ID NO: 68]
TSGQAGQHHHHHHGAYPYDVPDYAS [SEQ ID NO: 308]

TABLE 18
ET200-020

DNA Sequence
(light chain variable region scFv linker heavy chain variable region His tag + HA tag)
cagtctgtcgtgacgcagccgccctcagtgtctgcggccccaggacagaaggtcaccat
ctcctgctctggaagcacctccaacattggaaataatgatgtatcctggtaccagcagc
tcccaggaacagcccccaaactcctcatttatgacaataataagcgaccctcagggatt
cctgaccgattctctggctccaagtctggcacgtcagccaccctgggcatcaccggact
ccagactggggacgaggccgattattactgcggaacatgggatagcagcgtgagtgctt
cttgggtcttcggcagagggaccaagctgaccgtcctaggt [SEQ ID NO: 69]
tctagaggtggtggtggtagcggcggcggcggctctggtggtggtggatccctcgagatggcc [SEQ ID NO: 305]
caggtgcagctggtgcagtctggagctgaggtgaagaagcctggggcctcagtgaaggt
ctcctgcaaggcttctggttacacctttaccagctatggtatcagctgggtgcgacagg
cccctggacaagggcttgagtggatgggatggatcagcgcttacaatggtaacacaaac
tatccacagaagctccagggcagagtcaccatgaccacagacccatccacgagcacagc
ctacatggagctgaggagcctgagatctgacgacacggccgtgtattactgtgcgcgct
ctatgacttctttcgattactggggtcaaggtactctggtgaccgtctcctca [SEQ ID NO: 70]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVVTQPPSVSAAPGQKVTISCSGSTSNIGNNDVSWYQQLPGTAPKLLIYDNNKRPSGI
PDRFSGSKSGTSATLGITGLQTGDEADYYCGTWDSSVSASWVFGRGTKLTVLG [SEQ ID NO: 71]
SRGGGGSGGGGSGGGGSLEMA [SEQ ID NO: 307]
QVQLVQSGAEVKKPGASVKVSCKASGYTFTSYGISWVRQAPGQGLEWMGWISAYNGNT
NYPQKLQGRVTMTTDPSTSTAYMELRSLRSDDTAVYYCARSMTSFDYWGQGTLVTVSS
[SEQ ID NO: 72]
TSGQAGQHHHHHHGAYPYDVPDYAS [SEQ ID NO: 308]

TABLE 19
ET200-021

DNA Sequence
(light chain variable region scFv linker heavy chain variable region His tag + HA tag)
cagtctgtgttgacgcagccgccctcagtgtctgcggccccaggacagaaggtcaccat
ctcctgctctggaagcaactccaacattgggaataattatgtatcctggtatcagcaac
tcccagggacagcccccaaactcctcatttatgacaataataagcgaccctcagggatt
cctgaccgattctctggctccaggtctggcacgtcagccaccctgggcatcaccggact
ccagactggggacgaggccgattattactgcggaacatggaataccactgtgactcctg
gctatgtcttcggaactgggaccaaggtcaccgtcctaggt [SEQ ID NO: 73]
tctagaggtggtggtggtagcggcggcggcggctctggtggtggtggatccctcgagatggcc [SEQ ID NO: 305]
gaagtgcagctggtgcagtctggagctgaggtgaagaagcctggggcctcagtgaaggt
ctcctgcaaggcttctggttacacctttaccagctatggtatcagctgggtgcgacagg
cccctggacaagggcttgagtggatgggatggatcagcgcttacaatggtaacacaaac
tatgcacagaagctccagggcagagtcaccatgaccacagacacatccacgagcacagc
ctacatggagctgaggagcctgagatctgacgacaccgccatgtattactgtgcgcgct
ctgtttacgacctggatacttggggtcaaggtactctggtgaccgtctcctca [SEQ ID NO: 74]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPSVSAAPGQKVTISCSGSNSNIGNNYVSWYQQLPGTAPKLLIYDNNKRPSGI
PDRFSGSRSGTSATLGITGLQTGDEADYYCGTWNTTVTPGYVFGTGTKVTVLG [SEQ ID NO: 75]
SRGGGGSGGGGSGGGGSLEMA [SEQ ID NO: 307]
EVQLVQSGAEVKKPGASVKVSCKASGYTFTSYGISWVRQAPGQGLEWMGWISAYNGNT
NYAQKLQGRVTMTTDTSTSTAYMELRSLRSDDTAMYYCARSVYDLDTWGQGTLVTVSS
[SEQ ID NO: 76]
TSGQAGQHHHHHHGAYPYDVPDYAS [SEQ ID NO: 308]

TABLE 20
ET200-022

DNA Sequence
(light chain variable region scFv linker heavy chain variable region His tag + HA tag)
cagtctgtcgtgacgcagccgccctcagtgtctgcggccccaggacagaaggtcaccat
ctcctgctctggaagcagctccaacattgggaataattatgtatcctggtaccagcagc
tcccaggaacagcccccaaactcctcatttatgacaataataagcgaccctcagggatt
cctgaccgattctctggctccaagtctggcacgtcagccaccctgggcatcaccggact
ccagactggggacgaggccgattattactgcggaacatgggatagcagcctgggggccc
cttatgtcttcggaactgggaccaaggtcaccgtcctaggt [SEQ ID NO: 77]
tctagaggtggtggtggtagcggcggcggcggctctggtggtggtggatccctcgagatggcc [SEQ ID NO: 305]
gaggtgcagctggtgcagtcttggggaggctcggaacagcctggcaggtccctgagact
ctcctgtgcagcctctggattcacctttgatgattatgccatgcactgggtccggcaag
ctccagggaagggcctggagtgggtctcaggtattagttggaatagcggtagcataggc
tatgcggactctgtgaagggccgattcaccatctccagagacaacgccaagaattccct
gtatctgcaaatgaacagtctgagagctgaggacaccgccatgtattactgtgcgcgct
accgtcaggttggttctgcttacgattcttggggtcaaggtactctggtgaccgtctcc
tca [SEQ ID NO: 78]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVVTQPPSVSAAPGQKVTISCSGSSSNIGNNYVSWYQQLPGTAPKLLIYDNNKRPSGI
PDRFSGSKSGTSATLGITGLQTGDEADYYCGTWDSSLGAPYVFGTGTKVTVLG [SEQ ID NO: 79]
SRGGGGSGGGGSGGGGSLEMA [SEQ ID NO: 307]
EVQLVQSWGGSEQPGRSLRLSCAASGFTFDDYAMHWVRQAPGKGLEWVSGISWNSGSI
GYADSVKGRFTISRDNAKNSLYLQMNSLRAEDTAMYYCARYRQVGSAYDSWGQGTLVT
VSS [SEQ ID NO: 80]
TSGQAGQHHHHHHGAYPYDVPDYAS [SEQ ID NO: 308]

TABLE 21
ET200-023

DNA Sequence
(light chain variable region scFv linker heavy chain variable region His tag + HA tag)
ctgcctgtgctgactcagccaccctcggtgtcagtggccccaggaaagacggccaggat
tacctgtgggggaaacaacattggaagtaaaagtgtgcactggtatcagcagaagccag
gccaggcccctgtgctggtcgtctatgctgatagcgaccggccctcagggatccctgag
cgattctctggctccaactctgggaacacggccaccctgaccatcagcagggtcgaagc
cggggatgaggccgactattactgtcaggtgtgggatagtagtagttatcataattatg
tcttcggaactgggaccaaggtcaccgtcctaggt [SEQ ID NO: 81]
tctagaggtggtggtggtagcggcggcggcggctctggtggtggtggatccctcgagatggcc [SEQ ID NO: 305]
gaggtgcagctggtgcagtctggagctgaggtgaagaagcctggggcctcagtgaaggt
ctcctgcaaggcttctggttacacctttaccagctatggtatcagctgggtgcgacagg
cccctggacaagggcttgagtggatgggatggatcagcgcttacaatggtaacacaaac
tatgcacagaagctccagggcagagtcaccatgaccacagacacatccacgagcacagc
ctacatggagctgagcagcctgagatctgaggacaccgccatgtattactgtgcgcgct
actggggtttcggtgtttctgatcgttggggtcaaggtactctggtgaccgtctcctca [SEQ ID NO: 82]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
LPVLTQPPSVSVAPGKTARITCGGNNIGSKSVHWYQQKPGQAPVLVVYADSDRPSGIPE
RFSGSNSGNTATLTISRVEAGDEADYYCQVWDSSSYHNYVFGTGTKVTVLG [SEQ ID NO: 83]
SRGGGGSGGGGSGGGGSLEMA [SEQ ID NO: 307]
EVQLVQSGAEVKKPGASVKVSCKASGYTFTSYGISWVRQAPGQGLEWMGWISAYNGNT
NYAQKLQGRVTMTTDTSTSTAYMELSSLRSEDTAMYYCARYWGFGVSDRWGQGTLVTV
SS [SEQ ID NO: 84]
TSGQAGQHHHHHHGAYPYDVPDYAS [SEQ ID NO: 308]

TABLE 22
ET200-024

DNA Sequence
(light chain variable region scFv linker heavy chain variable region His tag + HA tag)
aattttatgctgactcagccccactctgtgtcggagtctccggggaagacggtaaccat
ctcctgcaccggcagcagtggcagcattgccagcaactatgtgcagtggtaccagcagc
gcccgggcagtgcccccaccactgtgatctatgaggataaccaaagaccctctggggtc
cccgatcggttctctggctccatcgacagctcctccaactctgcctccctcaccatctc
tggactgaagactgaggacgaggctgactactactgtcagtcttatgacagcagcaatc
tttgggtgttcggcggagggaccaagctgaccgtcctaggt [SEQ ID NO: 85]
tctagaggtggtggtggtagcggcggcggcggctctggtggtggtggatccctcgagatggcc [SEQ ID NO: 305]
cagatgcagctggtgcagtctggggctgaggtgaagaagcctgggtcctcggtgaaggt
ctcctgcaaggcttctggaggcaccttcagcagctatgctatcagctgggtgcgacagg
cccctggacaagggcttgagtggatgggagggatcatccctatctttggtacagcaaac
tacgcacagaagttccagggcagagtcacgattaccgcggacgaatccacgagcacagc
ctacatggagctgagcagcctgagatctgaggacactgccgtgtattactgtgcgcgct
acaactactactactacgattcttggggtcaaggtactctggtgaccgtctcctca
[SEQ ID NO: 86]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSESPGKTVTISCTGSSGSIASNYVQWYQQRPGSAPTTVIYEDNQRPSGV
PDRFSGSIDSSSNSASLTISGLKTEDEADYYCQSYDSSNLWVFGGGTKLTVLG [SEQ ID NO: 87]
SRGGGGSGGGGSGGGGSLEMA [SEQ ID NO: 307]
QMQLVQSGAEVKKPGSSVKVSCKASGGTFSSYAISWVRQAPGQGLEWMGGIIPIFGTA
NYAQKFQGRVTITADESTSTAYMELSSLRSEDTAVYYCARYNYYYYDSWGQGTLVTVSS [SEQ ID NO: 88]
TSGQAGQHHHHHHGAYPYDVPDYAS [SEQ ID NO: 308]

TABLE 23
ET200-025

DNA Sequence
(light chain variable region scFv linker heavy chain variable region His tag + HA tag)
gacatccagatgacccagtctccatcctccctgtctgcatctgtaggagacagagtcac
catcacttgccgggcaagtcagagcattagcagctatttaaattggtatcagcagaaac
cagggaaagcccctaagctcctgatctatgctgcatccagtttgcaaagtggggtccca
tcaaggttcagtggcagtggatctgggacagatttcactctcaccatcagcagtctgca
acctgaagattttgcaacttactactgtcaacagagttacagtaccccattcactttcg
gccctgggaccaaagtggatatcaaacgt [SEQ ID NO: 89]
tctagaggtggtggtggtagcggcggcggcggctctggtggtggtggatccctcgagatggcc [SEQ ID NO: 305]
gaggtgcagctggtgcagtctggggctgaggtgaagaagcctgggtcctcggtgaaggt
ctcctgcaaggcttctggaggcaccttcagcagctatgctatcagctgggtgcgacagg
cccctggacaagggcttgagtggatgggagggatcatccctatctttggtacagcaaac
tacgcacagaagttccagggcagagtcacgattaccgcggacgaatccacgagcacagc
ctacatggagctgagcagcctgagatctgaggacaccgccatgtattactgtgcgcgct
actggggttacgactcttacgatgaatggggtcaaggtactctggtgaccgtctcctca [SEQ ID NO: 90]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
DIQMTQSPSSLSASVGDRVTITCRASQSISSYLNWYQQKPGKAPKLLIYAASSLQSGVP
SRFSGSGSGTDFTLTISSLQPEDFATYYCQQSYSTPFTFGPGTKVDIKR [SEQ ID NO: 91]
SRGGGGSGGGGSGGGGSLEMA [SEQ ID NO: 307]
EVQLVQSGAEVKKPGSSVKVSCKASGGTFSSYAISWVRQAPGQGLEWMGGIIPIFGTA
NYAQKFQGRVTITADESTSTAYMELSSLRSEDTAMYYCARYWGYDSYDEWGQGTLVTVSS [SEQ ID NO: 92]
TSGQAGQHHHHHHGAYPYDVPDYAS [SEQ ID NO: 308]

TABLE 24
ET200-026

DNA Sequence
(light chain variable region scFv linker heavy chain variable region His tag + HA tag)
aattttatgctgactcagccccactctgtgtcggagtctccggggaagacggtaaccat
ctcctgcaccggcagcagtggcagcattgccagcaactatgtgcagtggtaccagcagc
gcccgggcagtgcccccaccactgtgatctatgaggataaccaaagaccctctggggtc
cctgatcggttctctggctccatcgacagctcctccaactctgcctccctcaccatctc
tggactgaagactgaggacgaggctgactactactgtcagtcttatgatagcagcaatt
gggtgttcggcggagggaccaagctgaccgtcctaggt [SEQ ID NO: 93]
tctagaggtggtggtggtagcggcggcggcggctctggtggtggtggatccctcgagatggcc [SEQ ID NO: 305]
gaggtccagctggtgcagtctggggctgaggtgaagaagcctgggtcctcggtgaaggt
ctcctgcaaggcttctggaggcaccttcagcagctatgctatcagctgggtgcgacagg
cccctggacaagggcttgagtggatgggagggatcatccctatctttggtacagcaaac
tacgcacagaagttccagggcagagtcacgattaccgcggacgaatccacgagcacagc
ctacatggagctgagcagcctgagatctgaggacacggccgtgtattactgtgcgcgca
acaaccattactacaacgattactggggtcaaggtactctggtgaccgtctcctca [SEQ ID NO: 94]
ACTAGTGGCCAGGCCGGCCAGCACCATCACCATCACCATGGCGCATACCCGTACGACGTTC
CGGACTACGCTTCT [SEQ ID NO: 306]
Amino Acid Sequence
(light chain variable region scFv linker heavy chain variable region His tag + HA tag)
NFMLTQPHSVSESPGKTVTISCTGSSGSIASNYVQWYQQRPGSAPTTVIYEDNQRPSGV
PDRFSGSIDSSSNSASLTISGLKTEDEADYYCQSYDSSNWVFGGGTKLTVLG [SEQ ID NO: 95]
SRGGGGSGGGGSGGGGSLEMA [SEQ ID NO: 307]
EVQLVQSGAEVKKPGSSVKVSCKASGGTFSSYAISWVRQAPGQGLEWMGGIIPIFGTA
NYAQKFQGRVTITADESTSTAYMELSSLRSEDTAVYYCARNNHYYNDYWGQGTLVTVSS [SEQ ID NO: 96]
TSGQAGQHHHHHHGAYPYDVPDYAS [SEQ ID NO: 308]

TABLE 25
ET200-027

DNA Sequence
(light chain variable region scFv linker heavy chain variable region His tag + HA tag)
cagtctgtgttgacgcagccgccctcagtgtctggggccccagggcagggggtcaccat
cccctgcactgggagcagctccaacatcggggcaggttatgatgtacactggtaccagc
agcttccagggacagcccccaaactcctcatctatggtaacaacaatcggccctcaggg
gtccctgaccgcttctctggctccaggtctggctcctcagcctccctggccatcactgg
gctccaggctgaggatgaggctgattattactgccagtcctatgacagcagcctgagtg
atgtggtattcggcggagggaccaaggtcaccgtcctaggt [SEQ ID NO: 97]
tctagaggtggtggtggtagcggcggcggcggctctggtggtggtggatccctcgagatggcc [SEQ ID NO: 305]
gaggtccagctggtgcagtctggggctgaggtgaagaagcctggggctacagtgaaaat
ctcctgcaaggtttctggatacaccttcaccgactactacatgcactgggtgcaacagg
cccctggaaaagggcttgagtggatgggacttgttgatcctgaagatggtgaaacaata
tacgcagagaagttccagggcagagtcaccataaccgcggacacgtctacagacacagc
ctacatggagctgagcagcctgagatctgaggacacggccgtgtattactgtgcgcgct
actggtcttactctttcgactacctgtacatgccggaaggtaacgattggtggggtcaa
ggtactctggtgaccgtctcctca [SEQ ID NO: 98]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPSVSGAPGQGVTIPCTGSSSNIGAGYDVHWYQQLPGTAPKLLIYGNNNRPSG
VPDRFSGSRSGSSASLAITGLQAEDEADYYCQSYDSSLSDVVFGGGTKVTVLG [SEQ ID NO: 99]
SRGGGGSGGGGSGGGGSLEMA [SEQ ID NO: 307]
EVQLVQSGAEVKKPGATVKISCKVSGYTFTDYYMHWVQQAPGKGLEWMGLVDPEDGET
IYAEKFQGRVTITADTSTDTAYMELSSLRSEDTAVYYCARYWSYSFDYLYMPEGNDWW
GQGTLVTVSS [SEQ ID NO: 100]
TSGQAGQHHHHHHGAYPYDVPDYAS [SEQ ID NO: 308]

TABLE 26
ET200-028

DNA Sequence
(light chain variable region scFv linker heavy chain variable region His tag + HA tag)
cagtctgtgttgactcagccacccgcagcgtctgggacccccggacagagagtcaccat
ctcttgttctgggggcgtctccaacatcgggagtggtgctctaaattggtaccagcaac
tcccaggaacggcccccaaactcctcatctatagttacaatcagcggccctcaggggtc
tctgaccgattctctggctccaggtctgccacctcagcctccctggccatcagtgggct
ccagtctgaggatgaggctgattattactgtgcaacctgggatgatagtgtgaatggtt
gggtgttcggcggagggaccaagctgaccgtcctaggt [SEQ ID NO: 101]
tctagaggtggtggtggtagcggcggcggcggctctggtggtggtggatccctcgagatggcc [SEQ ID NO: 305]
caggtccagctggtacagtctggagctgaggtgaagaagcctggggattcagtgaaggt
ctcctgcaagccttctggttacaattttctcaactatggtatcaactgggtgcgacagg
cccctggacaagggcttgagtggatgggatggattagcacttacaccggtaacacaaac
tatgcacagaagctgcagggcagagtcaccttcaccacagacacatccacgagcacagc
ctacatggagatgaggagcctgagatctgacgacacggccgtgtattactgtgcgcgcg
acctgtactactacgaaggtgttgattactggggtcaaggtactctggtgaccgtctcctca [SEQ ID NO: 102]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPAASGTPGQRVTISCSGGVSNIGSGALNWYQQLPGTAPKLLIYSYNQRPSGV
SDRFSGSRSATSASLAISGLQSEDEADYYCATWDDSVNGWVFGGGTKLTVLG [SEQ ID NO: 103]
SRGGGGSGGGGSGGGGSLEMA [SEQ ID NO: 307]
QVQLVQSGAEVKKPGDSVKVSCKPSGYNFLNYGINWVRQAPGQGLEWMGWISTYTGNTN
YAQKLQGRVTFTTDTSTSTAYMEMRSLRSDDTAVYYCARDLYYYEGVDYWGQGTLVTVSS [SEQ ID NO: 104]
TSGQAGQHHHHHHGAYPYDVPDYAS [SEQ ID NO: 308]

TABLE 27
ET200-029

DNA Sequence
(light chain variable region scFv linker heavy chain variable region His tag + HA tag)
caggctgtgctgactcagccaccctcagtgtcagtggccccaggaaagacggccagggt
tacctgtgggggaaacaacattggaagtgaaagtgtgcactggtaccagcagaagccag
gccaggcccctgtgttggtcatctattatgataccgaccggccctcagggatccctgag
cgattctctggctcccactctgggaccacggccaccctgaccatcagcagggtcgaagc
cggggatgaggccgactattactgtcaggtgtgggatagtagtagggatcatgtggtat
tcggcggagggaccaagctgaccgtcctaggt [SEQ ID NO: 105]
tctagaggtggtggtggtagcggcggcggcggctctggtggtggtggatccctcgagatggcc [SEQ ID NO: 305]
caggtgcagctggtgcagtctgggggaggcgtggtccagcctgggaggtccctgagact
ctcctgtgcggcctctggattcaccttcagtagctatgctatgcactgggtccgccagg
ctccaggcaagggactggagtgggtggcagttatatcatatgatggaagcaataaatac
tacgcagactccgtgaagggcctattcaccatctccagagacaattccaagaacacgct
gtatctgcaaatgaacagcctgagagctgaggacacggccgtgtattactgtgcgcgct
cttacttcacttctggtttctacgattactggggtcaaggtactctggtgaccgtctcctca [SEQ ID NO: 106]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QAVLTQPPSVSVAPGKTARVTCGGNNIGSESVHWYQQKPGQAPVLVIYYDTDRPSGIPE
RFSGSHSGTTATLTISRVEAGDEADYYCQVWDSSRDHVVFGGGTKLTVLG [SEQ ID NO: 107]
SRGGGGSGGGGSGGGGSLEMA [SEQ ID NO: 307]
QVQLVQSGGGVVQPGRSLRLSCAASGFTFSSYAMHWVRQAPGKGLEWVAVISYDGSN
KYYADSVKGLFTISRDNSKNTLYLQMNSLRAEDTAVYYCARSYFTSGFYDYWGQGTLVTVSS [SEQ ID NO: 108]
TSGQAGQHHHHHHGAYPYDVPDYAS [SEQ ID NO: 308]

TABLE 28
ET200-030

DNA Sequence
(light chain variable region scFv linker heavy chain variable region His tag + HA tag)
cagtctgtcgtgacgcagccgccctcagtgtctggggccccagggcagagggtcaccatctcctgcactgggagcagttccaacatcggggca
ggttatgatgtaaattggtatcagcagtttccaggaacagcccccaaactcctcatctatggtaacagcaatcggccctcaggggtccctgaccgat
tctctggctccaagtctggcacctcagcctccctggccatcactgggctccaggctgaggatgaggctgattattactgccagtcctatgacagca
gcctgagtggctcttatgtcttcggaactgggaccaaggtcaccgtcctaggt [SEQ ID NO: 109]
tctagaggtggtggtggtagcggcggcggcggctctggtggtggtggatccctcgagatggcc [SEQ ID NO: 305]
cagatgcagctggtgcagtctggggctgaggtgaagaagcctggggcctcagtgaaggtctcctgcaaggcttccggatacaccctcac
tgaattatccatgcactgggtgcgacaggctcctggaaaagggcttgagtggatgggaggttttgatcctgaagatggtgaaacaatcta
cgcacagaagttccagggcagagtcaccatgaccgaggacacatctacagacacagcctacatggagctgagcagcctgagatctga
ggacactgccgtgtattactgtgcgcgcatgtcttctatgtactacgattggggtcaaggtactctggtgaccgtctcctca
[SEQ ID NO: 110]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVVTQPPSVSGAPGQRVTISCTGSSSNIGAGYDVNWYQQFPGTAPKLLIYGNSNRPSGVPD
RFSGSKSGTSASLAITGLQAEDEADYYCQSYDSSLSGSYVFGTGTKVTVLG [SEQ ID NO: 111]
SRGGGGSGGGGSGGGGSLEMA [SEQ ID NO: 307]
QMQLVQSGAEVKKPGASVKVSCKASGYTLTELSMHWVRQAPGKGLEWMGGFDPED
GETIYAQKFQGRVTMTEDTSTDTAYMELSSLRSEDTAVYYCARMSSMYYDWGQGTL
VTVSS [SEQ ID NO: 112]
TSGQAGQHHHHHHGAYPYDVPDYAS [SEQ ID NO: 308]

TABLE 29
ET200-031

DNA Sequence
(light chain variable region scFv linker heavy chain variable region His tag + HA tag)
tcctatgtgctgactcagccaccctcagtgtcagtggccccaggaaagacggccaggattacctgtgggggaaacaacattggaagtaaaagtg
tgcactggtaccagcagaagccaggccaggcccctgtgctggtcatctattatgatagcgaccggccctcagggatccctgagcgattctctggc
tccaactctgggaacacggccaccctgaccatcagcagggtcgaagccggggatgaggccgactattactgtcaggtgtgggatagtagtagtg
attatgtcttcggaactgggaccaaggtcaccgtcctaggt [SEQ ID NO: 113]
tctagaggtggtggtggtagcggcggcggcggctctggtggtggtggatccctcgagatggcc [SEQ ID NO: 305]
gaggtgcagctggtggagactgggggaggcttggtcaagcctggagggtccctgagactctcctgtgcagcctctggattcaccgtcagt
gactactacatgagctggatccgccaggctccagggaagggcctggagtggatttcatacattagtggtagtggtaatagcatatactac
gcagactctgtgaagggccgattcaccatctccagggacaacgccaagaactcactggatctgcaaatgaccagcctgagagccgagg
acacggccgtatattactgtgcgcgctctactaaattcgattactggggtcaaggtactctggtgaccgtctcctca [SEQ ID NO: 114]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
SYVLTQPPSVSVAPGKTARITCGGNNIGSKSVHWYQQKPGQAPVLVIYYDSDRPSGIPERFS
GSNSGNTATLTISRVEAGDEADYYCQVWDSSSDYVFGTGTKVTVLG [SEQ ID NO: 115]
SRGGGGSGGGGSGGGGSLEMA [SEQ ID NO: 307]
EVQLVETGGGLVKPGGSLRLSCAASGFTVSDYYMSWIRQAPGKGLEWISYISGSGNSIY
YADSVKGRFTISRDNAKNSLDLQMTSLRAEDTAVYYCARSTKFDYWGQGTLVTVSS [SEQ ID NO: 116]
TSGQAGQHHHHHHGAYPYDVPDYAS [SEQ ID NO: 308]

TABLE 30
ET200-032

DNA Sequence
(light chain variable region scFv linker heavy chain variable region His tag + HA tag)
ctgcctgtgctgactcagccaccctcagcgtctgggacccccgggcagagggtcaccatctcttgttctggaagcagctccaacgtcggaagtta
cactgtaaactggtaccggcaactcccaggaacggcccccacactcctcatctataataataatcagcggccctcaggggtccctgaccgattctc
tgactccaagtctggcacctcggcctccctgaccattagtgggctccagcctgaggatgaggctgattattattgtgcagcatgggatgacaggct
gggtggttatgtcttcggaactgggaccaaggtcaccgtcctaggt [SEQ ID NO: 117]
tctagaggtggtggtggtagcggcggcggcggctctggtggtggtggatccctcgagatggcc [SEQ ID NO: 305]
gaggtgcagctggtgcagtctggagcagaggtgaaaaagccgggggagtctctgaagatctcctgtaagggttctggatacagctttac
caactactggatcggctgggtgcgccagatgcccgggaaaggcctggagtggatggggatcatctatcctggtgactctgataccagat
acagcccgtccttccaaggccaggtcaccatctcagccgacaagtccatcagcaccgcctacctacagtggagcagcctgaaggcctcg
gacaccgccatgtattactgtgcgcgctctactggttcttctcatatgtctgatgaatggggtcaaggtactctggtgaccgtctcctca
[SEQ ID NO: 118]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
LPVLTQPPSASGTPGQRVTISCSGSSSNVGSYTVNWYRQLPGTAPTLLIYNNNQRPSGVPDRF
SDSKSGTSASLTISGLQPEDEADYYCAAWDDRLGGYVFGTGTKVTVLG [SEQ ID NO: 119]
SRGGGGSGGGGSGGGGSLEMA [SEQ ID NO: 307]
EVQLVQSGAEVKKPGESLKISCKGSGYSFTNYWIGWVRQMPGKGLEWMGIIYPGDSD
TRYSPSFQGQVTISADKSISTAYLQWSSLKASDTAMYYCARSTGSSHMSDEWGQGTLV
TVSS [SEQ ID NO: 120]
TSGQAGQHHHHHHGAYPYDVPDYAS [SEQ ID NO: 308]

TABLE 31
ET200-033

DNA Sequence
(light chain variable region scFv linker heavy chain variable region His tag + HA tag)
aattttatgctgactcagccccactctgtgtcggagtctccggggaagacggtaaccatctcctgcaccggcagcagtggcagcattgccagcaa
ctatgtgcagtggtaccagcagcgcccgggcagtgcccccaccactgtgatctatgaggataaccaaagaccctctggggtccctgatcggttct
ctggctccatcgacagctcctccaactctgcctccctcaccatctctggactgaagactgaggacgaggctgactactactgtcagtcttatgatag
cagcaatcattgggtgttcggcggagggaccaagctgaccgtcctaggt [SEQ ID NO: 121]
tctagaggtggtggtggtagcggcggcggcggctctggtggtggtggatccctcgagatggcc [SEQ ID NO: 305]
caagtgcagctacagcagtggggcgcaggactgttgaagccttcggagaccctgtccctcacctgcgctgtctatggtgggtccttcagt
ggttactactggagctggatccgccagcccccagggaaggggctggagtggattggggagatcactcatagtggaaggtccaactaca
acccgtccctcaagagtcgagtcaccatatcagtagacacgtccaagaaccagttctccctgaagctgagctctgtgaccgccgcggac
acggccgtgtattactgtgcgcgctcttctatcatgtctgattactggggtcaaggtactctggtgaccgtctcctca [SEQ ID NO: 122]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSESPGKTVTISCTGSSGSIASNYVQWYQQRPGSAPTTVIYEDNQRPSGVPDR
FSGSIDSSSNSASLTISGLKTEDEADYYCQSYDSSNHWVFGGGTKLTVLG [SEQ ID NO: 123]
SRGGGGSGGGGSGGGGSLEMA [SEQ ID NO: 307]
QVQLQQWGAGLLKPSETLSLTCAVYGGSFSGYYWSWIRQPPGKGLEWIGEITHSGRS
NYNPSLKSRVTISVDTSKNQFSLKLSSVTAADTAVYYCARSSIMSDYWGQGTLVTVSS [SEQ ID NO: 124]
TSGQAGQHHHHHHGAYPYDVPDYAS [SEQ ID NO: 308]

TABLE 32
ET200-034

DNA Sequence
(light chain variable region scFv linker heavy chain variable region His tag + HA tag)
cagtctgtgttgacgcagccgccctcagtgtctggggccccagggcagagggtcaccatctcctgcactgggagcacctccaacatcggggca
ggttatgatgtacactggtaccagcagcttccaggaacagcccccaaactcctcatcaacaataacaggaatcggccctcaggggtccctgacc
gattctctggctccaagtctggcacgtcagccaccctgggcatcaccggactccagactggggacgaggccgattattactgcggaacatggga
tggcagcctgactggtgcagtgttcggcggagggaccaagctgaccgtcctaggt [SEQ ID NO: 125]
tctagaggtggtggtggtagcggcggcggcggctctggtggtggtggatccctcgagatggcc [SEQ ID NO: 305]
gaggtccagctggtgcagtctggggctgaggtgaagaagcctgggtcctcggtgaaggtctcatgcaaggcttctggaggcaccttcag
cagctatgctatcagctgggtgcgacaggcccctggacaagggcttgagtggatgggagggatcatccctatctttggtacagcaaacta
cgcacagaagttccagggcagagtcacgattaccgcggacgaatccacgagcacagcctacatggagctgagcagcctgagatctga
ggacacggccgtgtattactgtgcgcgcggttctgctctggaccattacgatcgttggggtcaaggtactctggtgaccgtctcctca
[SEQ ID NO: 126]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPSVSGAPGQRVTISCTGSTSNIGAGYDVHWYQQLPGTAPKLLINNNRNRPSGVPD
RFSGSKSGTSATLGITGLQTGDEADYYCGTWDGSLTGAVFGGGTKLTVLG [SEQ ID NO: 127]
SRGGGGSGGGGSGGGGSLEMA [SEQ ID NO: 307]
EVQLVQSGAEVKKPGSSVKVSCKASGGTFSSYAISWVRQAPGQGLEWMGGIIPIFGTA
NYAQKFQGRVTITADESTSTAYMELSSLRSEDTAVYYCARGSALDHYDRWGQGTLVT
VSS [SEQ ID NO: 128]
TSGQAGQHHHHHHGAYPYDVPDYAS [SEQ ID NO: 308]

TABLE 33
ET200-035

DNA Sequence
(light chain variable region scFv linker heavy chain variable region His tag + HA tag)
aattttatgctgactcagccccactctgtgtcggagtctccggggaagacggtaaccatctcctgcacccgcagcagtggcagcattgccagcaa
ctatgtgcagtggtaccagcagcgcccgggcagtgcccccaccactgtgatctatgaggataaccaaagaccctctggggtccctgatcggttct
ctggctccatcgacagctcctccaactctgcctccctcaccatctctggactgaagactgaggacgaggctgactactactgtcagtcttatgatag
caccaattgggtgttcggcggagggaccaagctgaccgtcctaggt [SEQ ID NO: 129]
tctagaggtggtggtggtagcggcggcggcggctctggtggtggtggatccctcgagatggcc [SEQ ID NO: 305]
caggtgcagctggtgcagtctggggctgaggtgaagaagcctgggtcctcggtgaaggtctcctgcaaggcttctggaggcaccttcag
cagctatgctatcagctgggtgcgacaggcccctggacaagggcttgagtggatgggagggatcatccctatctttggtacagcaaacta
cgcacagaagttccagggcagagtcacgattaccgcggacgaatccacgagcacagcctacatggagctgagcagcctgagatctga
ggacactgccgtgtattactgtgcgcgctacaactactacttcaacgattactggggtcaaggtactctggtgaccgtctcctca
[SEQ ID NO: 130]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSESPGKTVTISCTRSSGSIASNYVQWYQQRPGSAPTTVIYEDNQRPSGVPDRF
SGSIDSSSNSASLTISGLKTEDEADYYCQSYDSTNWVFGGGTKLTVLG [SEQ ID NO: 131]
SRGGGGSGGGGSGGGGSLEMA [SEQ ID NO: 307]
QVQLVQSGAEVKKPGSSVKVSCKASGGTFSSYAISWVRQAPGQGLEWMGGIIPIFGTA
NYAQKFQGRVTITADESTSTAYMELSSLRSEDTAVYYCARYNYYFNDYWGQGTLVTV
SS [SEQ ID NO: 132]
TSGQAGQHHHHHHGAYPYDVPDYAS [SEQ ID NO: 308]

TABLE 34
ET200-037

DNA Sequence
(light chain variable region scFv linker heavy chain variable region His tag + HA tag)
tcctatgtgctgactcagccaccctcagtgtcagtggccccaggaaagacggccaggattacctgtgggggaaacaacattggaagtaaaagtg
tgcactggtaccagcagaagccaggccaggcccctgtgctggtcatctattatgatagcgaccggccctcagggatccctgagcgattctctggc
tccaactctgggaacacggccaccctgaccatcagcagggtcgaagccggggatgaggccgactattactgtcaggtgtgggatagtagtagtg
atcatccttatgtcttcggaactgggaccaaggtcaccgtcctaggt [SEQ ID NO: 133]
tctagaggtggtggtggtagcggcggcggcggctctggtggtggtggatccctcgagatggcc [SEQ ID NO: 305]
cagatgcagctggtgcagtctggagctgaggtgaagaagcctggggcctcagtgaaggtctcctgcaaggcttctggttacacctttacc
agctatggtatcagctgggtgcgacaggcccctggacaagggcttgagtggatgggatggatcagcgcttacaatggtaacacaaacta
tgcacagaagctccagggcagagtcaccatgaccacagacacatccacgagcacagcctacatggagctgaggagcctgagatctga
cgacactgccgtgtattactgtgcgcgctctatgttcggtgctcatgattcttggggtcaaggtactctggtgaccgtctcctca
[SEQ ID NO: 134]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
SYVLTQPPSVSVAPGKTARITCGGNNIGSKSVHWYQQKPGQAPVLVIYYDSDRPSGIPERFS
GSNSGNTATLTISRVEAGDEADYYCQVWDSSSDHPYVFGTGTKVTVLG [SEQ ID NO: 135]
SRGGGGSGGGGSGGGGSLEMA [SEQ ID NO: 307]
QMQLVQSGAEVKKPGASVKVSCKASGYTFTSYGISWVRQAPGQGLEWMGWISAYNG
NTNYAQKLQGRVTMTTDTSTSTAYMELRSLRSDDTAVYYCARSMFGAHDSWGQGTL
VTVSS [SEQ ID NO: 136]
TSGQAGQHHHHHHGAYPYDVPDYAS [SEQ ID NO: 308]

TABLE 35
ET200-038

DNA Sequence
(light chain variable region scFv linker heavy chain variable region His tag + HA tag)
cagtctgtgttgacgcagccgccctcagtgtctggggccccagggcagagggtcaccatctcctgcactgggagcagctccaacatcggggca
ggttttgatgtacactggtaccagctacttccaggaacagcccccaaactcctcatctatgctaacagcaatcggccctcaggggtccctgaccga
ttctctggctccaagtctggcacctcagcctccctggccatcactgggctcctggctgaggatgaggctgattattactgccagtcctatgacagca
gcctgagtggtgtggtattcggcggagggaccaagctgaccgtcctaggt [SEQ ID NO: 137]
tctagaggtggtggtggtagcggcggcggcggctctggtggtggtggatccctcgagatggcc [SEQ ID NO: 305]
caggtgcagctggtgcaatctggggctgaggtgaagaagcctgggtcctcggtgaaggtctcctgcaaggcttctggaggcaccttcag
cagctatgctatcagctgggtgcgacaggcccctggacaagggcttgagtggatgggagggatcatccctatctttggtacagcaaacta
cgcacagaagttccagggcagagtcacgattaccgcggacgaatccacgagcacagcctacatggagctgagcagcctgagatctga
ggacactgccgtgtattactgtgcgcgcggtgcttctttcgaccgtcatgataactggggtcaaggtactctggtgaccgtctcctca
[SEQ ID NO: 138]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPSVSGAPGQRVTISCTGSSSNIGAGFDVHWYQLLPGTAPKLLIYANSNRPSGVPD
RFSGSKSGTSASLAITGLLAEDEADYYCQSYDSSLSGVVFGGGTKLTVLG [SEQ ID NO: 139]
SRGGGGSGGGGSGGGGSLEMA [SEQ ID NO: 307]
QVQLVQSGAEVKKPGSSVKVSCKASGGTFSSYAISWVRQAPGQGLEWMGGIIPIFGTA
NYAQKFQGRVTITADESTSTAYMELSSLRSEDTAVYYCARGASFDRHDNWGQGTLVT
VSS [SEQ ID NO: 140]
TSGQAGQHHHHHHGAYPYDVPDYAS [SEQ ID NO: 308]

TABLE 36
ET200-039

DNA Sequence
(light chain variable region scFv linker heavy chain variable region His tag + HA tag)
aattttatgctgactcagccccactctgtgtcggagtctccggggaagacggtaaccatctcctgcacccgcagcagtggcagcattgccagcaa
ctatgtgcagtggtaccagcagcgcccgggcagttcccccaccactgtgatctatgaggataaccaaagaccctctggggtccctgatcggttct
ctggctccatcgacagctcctccaactctgcctccctcaccatctctggactgaagactgaggacgaggctgactactactgtcagtcttatgatag
cagcaattgggtgttcggcggagggaccaagctgaccgtcctaggt [SEQ ID NO: 141]
tctagaggtggtggtggtagcggcggcggcggctctggtggtggtggatccctcgagatggcc [SEQ ID NO: 305]
gaggtccagctggtgcagtctggggctgaggtgaagaagcctgggtcctcggtgaaggtctcctgcaaggcttctggaggcaccttcag
cagctatgctatcagctgggtgcgacaggcccctggacaagggcttgagtggatgggagggatcatccctatctttggtacagcaaacta
cgcacagaagttccagggcagagtcacgattaccgcggacgaatccacgagcacagcctacatggagctgagcagcctgagatctga
ggacacggccgtgtattactgtgcgcgctctaactactactacaacgattactggggtcaaggtactctggtgaccgtctcctca
[SEQ ID NO: 142]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSESPGKTVTISCTRSSGSIASNYVQWYQQRPGSSPTTVIYEDNQRPSGVPDRF
SGSIDSSSNSASLTISGLKTEDEADYYCQSYDSSNWVFGGGTKLTVLG [SEQ ID NO: 143]
SRGGGGSGGGGSGGGGSLEMA [SEQ ID NO: 307]
EVQLVQSGAEVKKPGSSVKVSCKASGGTFSSYAISWVRQAPGQGLEWMGGIIPIFGTA
NYAQKFQGRVTITADESTSTAYMELSSLRSEDTAVYYCARSNYYYNDYWGQGTLVTVSS [SEQ ID NO: 144]
TSGQAGQHHHHHHGAYPYDVPDYAS [SEQ ID NO: 308]

TABLE 37
ET200-040

DNA Sequence
(light chain variable region scFv linker heavy chain variable region His tag + HA tag)
cagtctgtgttgacgcagccgccctcagtgtctggggccccagggcagagggtcaccatctcctgcactgggagcagctccaacatcggggca
ggttatgatgtacactggtaccagcagcttccaggaacagcccccaaactcctcatctatggtaacagcaatcggccctcaggggtccctgaccg
attctctggctccaagtctggcacctcagcctccctggccatcactgggctccaggctgaggatgaggctgattattactgccagtcctatgacagc
agcctgagtggttatgtcttcggaactgggaccaaggtcaccgtcctaggt [SEQ ID NO: 145]
tctagaggtggtggtggtagcggcggcggcggctctggtggtggtggatccctcgagatggcc [SEQ ID NO: 305]
caggtgcagctggtgcagtctggggctgaggtgaagaagcctggggcctcagtgaaggtctcctgcaaggtttccggatacaccctcac
tgaattatccatgcactgggtgcgacaggctcctggaaaagggcttgagtggatgggaggttttgatcctgaagatggtgaaacaatcta
cgcacagaagttccagggcagagtcaccatgaccgaggacacatctacagacacagcctacatggagctgagcagcctgagatctga
ggacactgccgtgtattactgtgcgcgctactctggtgtttactacgattggggtcaaggtactctggtgaccgtctcctca
[SEQ ID NO: 146]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVLTQPPSVSGAPGQRVTISCTGSSSNIGAGYDVHWYQQLPGTAPKLLIYGNSNRPSGVPD
RFSGSKSGTSASLAITGLQAEDEADYYCQSYDSSLSGYVFGTGTKVTVLG [SEQ ID NO: 147]
SRGGGGSGGGGSGGGGSLEMA [SEQ ID NO: 307]
QVQLVQSGAEVKKPGASVKVSCKVSGYTLTELSMHWVRQAPGKGLEWMGGFDPED
GETIYAQKFQGRVTMTEDTSTDTAYMELSSLRSEDTAVYYCARYSGVYYDWGQGTLV
TVSS [SEQ ID NO: 148]
TSGQAGQHHHHHHGAYPYDVPDYAS [SEQ ID NO: 308]

TABLE 38
ET200-041

DNA Sequence
(light chain variable region scFv linker heavy chain variable region His tag + HA tag)
aattttatgctgactcagccccactctgtgtcggggtctccggggaagacggtaaccatctcctgcaccggcagcagtggcagcattgccgacaa
ctttgtgcagtggtaccagcagcgcccgggcggtgtccccaccactgtgatctttaatgatgacgaaagaccctctggcgtccctgatcggttctct
ggctccatcgacacctcctccaattctgcctccctcaccatctctggactgaagactgaggacgaggctgactactactgtcagtcttatgataataa
taatcgaggggtgttcggcggagggaccaagctgaccgtcctaggt [SEQ ID NO: 149]
tctagaggtggtggtggtagcggcggcggcggctctggtggtggtggatccctcgagatggcc [SEQ ID NO: 305]
caggtccagctggtgcagtctggggctgaggtgaagaagcctgggtcctcggtgaaggtctcctgcaaggcttctggaggcaccttcag
cagctatgctatcagctgggtgcgacaggcccctggacaagggcttgagtggatgggatggatgaaccctaacagtggtaacacaggc
tatgcacagaagttccagggcagagtcaccatgaccaggaacacctccataagcacagcctacatggagctgagcaacctgagatctg
aggacacggccgtgtattactgtgcgcgctactactcttacggttacgattggggtcaaggtactctggtgaccgtctcctca
[SEQ ID NO: 150]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSGSPGKTVTISCTGSSGSIADNFVQWYQQRPGGVPTTVIFNDDERPSGVPDR
FSGSIDTSSNSASLTISGLKTEDEADYYCQSYDNNNRGVFGGGTKLTVLG [SEQ ID NO: 151]
SRGGGGSGGGGSGGGGSLEMA [SEQ ID NO: 307]
QVQLVQSGAEVKKPGSSVKVSCKASGGTFSSYAISWVRQAPGQGLEWMGWMNPNSG
NTGYAQKFQGRVTMTRNTSISTAYMELSNLRSEDTAVYYCARYYSYGYDWGQGTLVT
VSS [SEQ ID NO: 152]
TSGQAGQHHHHHHGAYPYDVPDYAS [SEQ ID NO: 308]

TABLE 39
ET200-042

DNA Sequence
(light chain variable region scFv linker heavy chain variable region His tag + HA tag)
cagtctgtcgtgacgcagccgccctcagtgtctggggccccagggcagacggtcaccatctcctgcactgggggcagctccaacatcgggaca
ggttattttgtaaattggtaccagcaggttccaggaaaagcccccaaactcctcatcctgggtaacaataatcggccctcgggggtccctgaccga
ctctccggctccacgtccggcacctcagcctccctggccatcactgggctccaggctgaggatgagggtacttattactgccagtcctatgacagc
agcctgagtggttatgtcttcggaactgggaccaaggtcaccgtcctaggt [SEQ ID NO: 153]
tctagaggtggtggtggtagcggcggcggcggctctggtggtggtggatccctcgagatggcc [SEQ ID NO: 305]
caggtacagctgcagcagtcaggtccaggactggtgaagccctcgcagaccctctcactcacctgtggcatctccggggacagtgtctct
accaacagtgttgcttggcactggatcaggcagtccccatcgagaggccttgagtggctgggaaggacatactacaggtccaagtggtc
taatgactatggagtatctgtgaaaagtcgaatcaccatcatcccagacacatccaagaaccagttctccctgcagctgaactctgtgact
cccgaggacacggctgtgtattactgtgcgcgctcttcttcttggtaccagatcttcgattactggggtcaaggtactctggtgaccgtctcc
tca [SEQ ID NO: 154]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
QSVVTQPPSVSGAPGQTVTISCTGGSSNIGTGYFVNWYQQVPGKAPKLLILGNNNRPSGVPD
RLSGSTSGTSASLAITGLQAEDEGTYYCQSYDSSLSGYVFGTGTKVTVLG [SEQ ID NO: 155]
SRGGGGSGGGGSGGGGSLEMA [SEQ ID NO: 307]
QVQLQQSGPGLVKPSQTLSLTCGISGDSVSTNSVAWHWIRQSPSRGLEWLGRTYYRSK
WSNDYGVSVKSRITIIPDTSKNQFSLQLNSVTPEDTAVYYCARSSSWYQIFDYWGQGTL
VTVSS [SEQ ID NO: 156]
TSGQAGQHHHHHHGAYPYDVPDYAS [SEQ ID NO: 308]

TABLE 40
ET200-043

DNA Sequence
(light chain variable region scFv linker heavy chain variable region His tag + HA tag)
aattttatgctgactcagccccactctgtgtcggagtctccggggaagacggtaaccatctcctgcaccggcagcagcgacagcatagccaacaa
ctatgttcagtggtaccagcagcgcccgggcagtgcccccaccaatgtgatctacgaagatgtccaaagaccctctggggtccctgatcggttct
ctgggtccatcgacagctcctccaactctgcctccctcaccatctctggactgaagactgaggacgaggctgtctactattgtcagtcttatcatagc
gacaatcgttgggtgttcggcggcgggaccaagctgaccgtcctaggt [SEQ ID NO: 157]
tctagaggtggtggtggtagcggcggcggcggctctggtggtggtggatccctcgagatggcc [SEQ ID NO: 305]
caggtgcagctggtggagtctgggggaggcttggtacagcctggggggtccctgagactctcctgtgcagcctctggattcacctttagc
agctatgccatgagctgggtccgccaggctccagggaaggggctggagtgggtctcagctattagtggtagtggtggtagcacatacta
cgcagactccgtgaagggccggttcaccatctccagagacaattccaagaacacgctgtatctgcaaatgaacagcctgagagccgag
gacacggccgtatattactgtgcgcgctctggtgcttactgggactactctgtttacgatgaatggggtcaaggtactctggtgaccgtctc
ctca [SEQ ID NO: 158]
ACTAGTGGCCAGGCCGGCCAGCACCATCACCATCACCATGGCGCATACCCGTACGACGT
TCCGGACTACGCTTCT [SEQ ID NO: 306]
Amino Acid Sequence
(light chain variable region scFv linker heavy chain variable region His tag + HA tag)
NFMLTQPHSVSESPGKTVTISCTGSSDSIANNYVQWYQQRPGSAPTNVIYEDVQRPSGVPDR
FSGSIDSSSNSASLTISGLKTEDEAVYYCQSYHSDNRWVFGGGTKLTVLG [SEQ ID NO: 159]
SRGGGGSGGGGSGGGGSLEMA [SEQ ID NO: 307]
QVQLVESGGGLVQPGGSLRLSCAASGFTFSSYAMSWVRQAPGKGLEWVSAISGSGGST
YYADSVKGRFTISRDNSKNTLYLQMNSLRAEDTAVYYCARSGAYWDYSVYDEWGQGT
LVTVSS [SEQ ID NO: 160]
TSGQAGQHHHHHHGAYPYDVPDYAS [SEQ ID NO: 308]

TABLE 41
ET200-044

DNA Sequence
(light chain variable region scFv linker heavy chain variable region His tag + HA tag)
cagtctgtgttgactcagccaccctcagtgtccgtgtccccaggacagacagccaccatcgcctgttctggacataaattggggg
ataaatatgcttcctggtatcagcagaagtcgggccagtcccctgtgttgatcatctatcaggataataagcggccctcagggattc
ctgagcgattctctggctccaactctgggaacacagccactctgaccatcagcgggacccaggctctggatgaggctgactatta
ttgtcaggcgtgggacagtagtacttatgtggcattcggcggagggaccaagctgaccgtcctaggt [SEQ ID NO:
161]
tctagaggtggtggtggtagcggcggcggcggctctggtggtggtggatccctcgagatggcc [SEQ ID NO: 305]
caggtgcagctgcaggagtccggcccaggactggtgaagccttcggagaccctgtccctcacctgcgttgtctctggtgg
ctccatcagcagtagtaactggtggagctgggtccgccagcccccagggaaggggctggagtggattggggaaatctat
catagtgggagccccaactacaacccatccctcaagagtcgagtcaccatatcagtagacaagtccaagaaccagttct
ccctgaagctgagctctgtgaccgccgcggacacggccgtgtattactgtgcgcgcatgactactcatactttcggttacg
atgcttggggtcaaggtactctggtgaccgtctcctca [SEQ ID NO: 162]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSVSVSPGQTATIACSGHKLGDKYASWYQQKSGQSPVLIIYQDNKRPS
GIPERFSGSNSGNTATLTISGTQALDEADYYCQAWDSSTYVAFGGGTKLTVLG
[SEQ ID NO: 163]
SRGGGGSGGGGSGGGGSLEMA [SEQ ID NO: 307]
QVQLQESGPGLVKPSETLSLTCVVSGGSISSSNWWSWVRQPPGKGLEWIGEI
YHSGSPNYNPSLKSRVTISVDKSKNQFSLKLSSVTAADTAVYYCARMTTHTF
GYDAWGQGTLVTVSS [SEQ ID NO: 164]
TSGQAGQHHHHHHGAYPYDVPDYAS [SEQ ID NO: 308]

TABLE 42
ET200-045

DNA Sequence
(light chain variable region scFv linker heavy chain variable region His tag + HA tag)
cagcctgtgctgactcagccaccctcagtgtcagtggccccaggaaagacggccacgattacttgtgggggaaacaacattgg
aagtgaaagtgtgcactggtaccaccagaagccaggccaggcccctgtgttggtcatctatgatgatgccggccggccctcag
ggatccctgagcgattcactggctccaactctgggaacacggccaccctgaccatcagcagggtcgaagccggggatgaggc
cgactattactgtcaggtgtgggacagaaatagtgctcagtttgtcttcggacctgggaccaaggtcaccgtcctaggt [SEQ
ID NO: 165]
tctagaggtggtggtggtagcggcggcggcggctctggtggtggtggatccctcgagatggcc [SEQ ID NO: 305]
gaggtccagctggtgcagtctggagctgaggtgaagaagcctggggcctcagtgaaggtctcctgcaaggcttctggtt
acacctttaccagctatggtatcagctgggtgcgacaggcccctggacaagggcttgagtggatgggatggatcagcgc
ttacaatggtaacacaaactatgcacagaagctccagggcagagtcaccatgaccacagacacatccacgagcacagc
ctacatggagctgaggagcctgagatctgacgacacggccgtgtattactgtgcgcgcggtgttcatctggattggtggg
gtcaaggtactctggtgaccgtctcctca [SEQ ID NO: 166]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QPVLTQPPSVSVAPGKTATITCGGNNIGSESVHWYHQKPGQAPVLVIYDDAGRPS
GIPERFTGSNSGNTATLTISRVEAGDEADYYCQVWDRNSAQFVFGPGTKVTVLG
[SEQ ID NO: 167]
SRGGGGSGGGGSGGGGSLEMA [SEQ ID NO: 307]
EVQLVQSGAEVKKPGASVKVSCKASGYTFTSYGISWVRQAPGQGLEWMGW
ISAYNGNTNYAQKLQGRVTMTTDTSTSTAYMELRSLRSDDTAVYYCARGVH
LDWWGQGTLVTVSS [SEQ ID NO: 168]
TSGQAGQHHHHHHGAYPYDVPDYAS [SEQ ID NO: 308]

TABLE 43
ET200-069

DNA Sequence
(light chain variable region scFv linker heavy chain variable region His tag + HA tag)
cagtctgtcgtgacgcagccaccctcagcgtctgggacccccgggcagagggtcaccatctcttgttctggaagcagctccaac
atcggaagtaattatgtatactggtaccagcagctcccaggaacggcccccaaactcctcatctatagtaataatcagcggccctc
aggggtccctgaccgattctctggctccaagtctggcacctcagcctccctggccatcagtgggctccggtccgaggatgaggc
tgattattactgtgcagcatgggatgacagcctgagtggttatgtcttcggaactgggaccaagctgaccgtcctaggt [SEQ
ID NO: 169]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccctcacctgcgctgtctatggtgg
gtccttcagtggttactactggagctggatccgccagcccccagggaaggggctggagtggattggggaaatcaatcat
agtggaagcaccaactacaacccgtccctcaagagtcgagtcaccatatcagtagacacgtccaagaaccagttctccc
tgaagctgagctctgtgaccgccgcggacacggccgtgtattactgtgcgcgcctgtacgaaggtggttaccatggttgg
ggttcttggctgtcttctgattcttggggtcaaggtactctggtgaccgtctcctca [SEQ ID NO: 170]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVVTQPPSASGTPGQRVTISCSGSSSNIGSNYVYWYQQLPGTAPKLLIYSNNQRP
SGVPDRFSGSKSGTSASLAISGLRSEDEADYYCAAWDDSLSGYVFGTGTKLTVL
G [SEQ ID NO: 171]
SRGGGGSGGGGSGGGGSLEMA [SEQ ID NO: 307]
QVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCARLYEGGY
HGWGSWLSSDSWGQGTLVTVSS [SEQ ID NO: 172]
TSGQAGQHHHHHHGAYPYDVPDYAS [SEQ ID NO: 308]

TABLE 44
ET200-078

DNA Sequence
(light chain variable region scFv linker heavy chain variable region His tag + HA tag)
cagtctgtgttgactcagccaccctcagcgtctgggacccccgggcagagggtcaccatctcttgttctggaagcagctccaaca
tcggaagtaatactgtaaactggtaccagcagctcccaggaacggcccccaaactcctcatctatagtaataatcagcggccctc
aggggtccctgaccgattctctggctccaagtctggcacctcagcctccctggccatcagtgggctccagtctgaggatgaggct
gattattactgtgcagcatgggatgacagcctgaatggttattgggtgttcggcggagggaccaagctgaccgtcctaggt
[SEQ ID NO: 173]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccctcacctgcgctgtctatggtgg
gtccttcagtggttactactggagctggatccgccagcccccagggaaggggctggagtggattggggaaatcaatcat
agtggaagcaccaactacaacccgtccctcaagagtcgagtcaccatatcagtagacacgtccaagaaccagttctccc
tgaagctgagctctgtgaccgccgcggacacggctgtgtattactgtgcgcgcgaaggggcatttgatgcttttgatatct
ggggccaagggacaatggtcaccgtctcttca [SEQ ID NO: 174]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSASGTPGQRVTISCSGSSSNIGSNTVNWYQQLPGTAPKLLIYSNNQRP
SGVPDRFSGSKSGTSASLAISGLQSEDEADYYCAAWDDSLNGYWVFGGGTKLTV
LG [SEQ ID NO: 175]
SRGGGGSGGGGSGGGGSLEMA [SEQ ID NO: 307]
QVQLQQWGAGLLKPSETLSLTCAVYGGSFSGYYWSWIRQPPGKGLEWIGEINHS
GSTNYNPSLKSRVTISVDTSKNQFSLKLSSVTAADTAVYYCAREGAFDAFDIWG
QGTMVTVS [SEQ ID NO: 176]
TSGQAGQHHHHHHGAYPYDVPDYAS [SEQ ID NO: 308]

TABLE 45
ET200-079

DNA Sequence
(light chain variable region scFv linker heavy chain variable region His tag + HA tag)
tcctatgagctgactcagccaccctcagcgtctgggacccccgggcagagggtcaccatctcttgttctggaagcagctccaac
atcggaagtaattatgtatactggtaccagcagctcccaggaacggcccccaaactcttcatctataggaataatcagcggccctc
aggggtccctgaccgattctctggctccaagtctggcacctcagcctccctggccatcagtgggctccggtccgaggatgaggc
tgattattactgtgcagcatgggatgacagcctgagtggttatctcttcggaactgggaccaaggtcaccgtcctaggt [SEQ
ID NO: 177]
tctagaggtggtggtggtagcggcggcggcggctctggtggtggtggatccctcgagatggcc [SEQ ID NO: 305]
gaggtgcagctggtggagtctgggggaggcttggtacagcctggcaggtccctgagactctcctgtgcagcctctggatt
cacctttgatgattatgccatgcactgggtccggcaagctccagggaagggcctggagtgggtctcaggtattagttgga
atagtggtagcataggctatgcggactctgtgaagggccgattcaccatctccagagacaacgccaagaactccctgtat
ctgcaaatgaacagtctgagagctgaggacacggccttgtattactgtgcaaatggcgactccaactactactacggtat
ggacgtctggggccaagggaccacggtcaccgtctcctca [SEQ ID NO: 178]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SYELTQPPSASGTPGQRVTISCSGSSSNIGSNYVYWYQQLPGTAPKLFIYRNNQRP
SGVPDRFSGSKSGTSASLAISGLRSEDEADYYCAAWDDSLSGYLFGTGTKVTVL
G [SEQ ID NO: 179]
SRGGGGSGGGGSGGGGSLEMA [SEQ ID NO: 307]
EVQLVESGGGLVQPGRSLRLSCAASGFTFDDYAMHWVRQAPGKGLEWVSG
ISWNSGSIGYADSVKGRFTISRDNAKNSLYLQMNSLRAEDTALYYCANGDSN
YYYGMDVWGQGTTVTVSS [SEQ ID NO: 180]
TSGQAGQHHHHHHGAYPYDVPDYAS [SEQ ID NO: 308]

TABLE 46
ET200-081

DNA Sequence
(light chain variable region scFv linker heavy chain variable region His tag + HA tag)
cagtctgccctgactcagcctgcctccgtgtccgggtctcctggacagtcgatcaccatctcctgcactggaaccagcagtgaca
ttggtggttataactatgtctcctggtaccaacaacacccaggcaaagcccccaaactcatgatttatgatgtcagtaatcggccct
caggggtttctaatcgcttctctggctccaagtctggcaacacggcctccctgaccatctctgggctccaggctgaggacgaggc
tgattattactgcatctcatatacacgcacctggaacccctatgtcttcgggagtgggaccaaggtcaccgtcctaggt [SEQ
ID NO: 181]
tctagaggtggtggtggtagcggcggcggcggctctggtggtggtggatccctcgagatggcc [SEQ ID NO: 305]
gaggtgcagctggtgcagtctgggggaggcgtggtacagcctggggggtccctgagactctcctgtgcagcctctggatt
cacctttgatgattatgccatgcactgggtccgtcaagctccagggaagggtctggagtgggtctctcttattagtgggga
tggtggtagcacatactatgcagactctgtgaagggccgattcaccatctccagagacaacagcaaaaactccctgtatc
tgcaaatgaacagtctgagaactgaggacaccgccttgtattactgtgcaaaagatcgggcagcagctggctactacta
ctacggtatggacgtctggggccaagggaccacggtcaccgtctcctca [SEQ ID NO: 182]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSALTQPASVSGSPGQSITISCTGTSSDIGGYNYVSWYQQHPGKAPKLMIYDVSN
RPSGVSNRFSGSKSGNTASLTISGLQAEDEADYYCISYTRTWNPYVFGSGTKVTV
LG [SEQ ID NO: 183]
SRGGGGSGGGGSGGGGSLEMA [SEQ ID NO: 307]
EVQLVQSGGGVVQPGGSLRLSCAASGFTFDDYAMHWVRQAPGKGLEWVSL
ISGDGGSTYYADSVKGRFTISRDNSKNSLYLQMNSLRTEDTALYYCAKDRAA
AGYYYYGMDVWGQGTTVTVSS [SEQ ID NO: 184]
TSGQAGQHHHHHHGAYPYDVPDYAS [SEQ ID NO: 308]

TABLE 47
ET200-097

DNA Sequence
(light chain variable region scFv linker heavy chain variable region His tag + HA tag)
ctgcctgtgctgactcagccaccctcagtgtccgtgtccccaggacagacagccatcatcacctgctctggagataaattggggg
aaaaatatgtttcctggtatcagcagaagccaggccagtcccctgtactggtcatcgatcaagataccaggaggccctcagggat
ccctgagcgattctctggctccaactctgggaccacagccactctgaccatcagcgggacccaggctatggatgaggctgacta
ttactgtcaggcgtgggacaggggtgtggtattcggcggagggaccaagctgaccgtcctaggt [SEQ ID NO: 185]
tctagaggtggtggtggtagcggcggcggcggctctggtggtggtggatccctcgagatggcc [SEQ ID NO: 305]
gaggtgcagctggtggagtctgggggagacttggtacagcctggcaggtccctgagactctcctgtgcagcctctggatt
cacctttaatgattatgccatgcactgggtccggcaagctccagggaagggcctggagtgggtctcaggtattagttgga
gtggtaataacataggctatgcggactctgtgaagggccgattcaccatctccagagacaacgccaagaactccctgtat
ctgcaaatgaacagtctgagagctgaggacacggccttgtattactgtgcaaaagatagtatacggtatggcatcacctg
gggaggttttgactactggggccagggaaccctggtcaccgtctcctca [SEQ ID NO: 186]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
LPVLTQPPSVSVSPGQTAIITCSGDKLGEKYVSWYQQKPGQSPVLVIDQDTRRPS
GIPERFSGSNSGTTATLTISGTQAMDEADYYCQAWDRGVVFGGGTKLTVLG
[SEQ ID NO: 187]
SRGGGGSGGGGSGGGGSLEMA [SEQ ID NO: 307]
EVQLVESGGDLVQPGRSLRLSCAASGFTFNDYAMHWVRQAPGKGLEWVSG
ISWSGNNIGYADSVKGRFTISRDNAKNSLYLQMNSLRAEDTALYYCAKDSIR
YGITWGGFDYWGQGTLVTVSS [SEQ ID NO: 188]
TSGQAGQHHHHHHGAYPYDVPDYAS [SEQ ID NO: 308]

TABLE 48
ET200-098

DNA Sequence
(light chain variable region scFv linker heavy chain variable region His tag + HA tag)
cagcctgtgctgactcagccaccctcggtgtccaagggcttgagacagaccgccacactcacctgcactgggaacagcaacaa
tgttggcaacctaggagtagcttggctgcagcagcaccagggccaccctcccaaactcctatcctacaggaataacaaccggc
cctcagggatctcagagagattatctgcatccaggtcaggaaacacagcctccctgaccattactggactccagcctgaggacg
aggctgactattactgctcagcatgggacagtagcctcagtgcttgggtgttcggcggagggaccaagctgaccgtcctaggt
[SEQ ID NO: 189]
tctagaggtggtggtggtagcggcggcggcggctctggtggtggtggatccctcgagatggcc [SEQ ID NO: 305]
gaggtgcagctggtggagtctgggggagtcgtggtacagcctggggggtccctgagactctcctgtgcagcctctggatt
cacctttgatgattatgccatgcactgggtccgtcaagctccggggaagggtctggagtgggtctctcttattaattgggat
ggtggtagcacctactatgcagactctgtgaagggtcgattcaccatctccagagacaacagcaaaaactccctgtatct
gcaaatgaacagtctgagagctgaggacaccgccttgtattactgtgcaaaagggatgggcctgagggcgtttgactac
tggggccagggaaccctggtcaccgtctcctca [SEQ ID NO: 190]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QPVLTQPPSVSKGLRQTATLTCTGNSNNVGNLGVAWLQQHQGHPPKLLSYRNN
NRPSGISERLSASRSGNTASLTITGLQPEDEADYYCSAWDSSLSAWVFGGGTKLT
VLG [SEQ ID NO: 191]
SRGGGGSGGGGSGGGGSLEMA [SEQ ID NO: 307]
EVQLVESGGVVVQPGGSLRLSCAASGFTFDDYAMHWVRQAPGKGLEWVSL
INWDGGSTYYADSVKGRFTISRDNSKNSLYLQMNSLRAEDTALYYCAKGMG
LRAFDYWGQGTLVTVSS [SEQ ID NO: 192]
TSGQAGQHHHHHHGAYPYDVPDYAS [SEQ ID NO: 308]

TABLE 49
ET200-099

DNA Sequence
(light chain variable region scFv linker heavy chain variable region His tag + HA tag)
cagtctgtgttgactcagccaccctcagcgtctgggacccccgggcagagggtcaccatctcctgttctggaagcagctccaac
atcggaagtaatactgtaaactggtaccagcagctcccaggaacggcccccaaactcctcatctatagtaatgatcagcggccct
caggggtccctgaccgattctctggctccaagtccggcacctcagcctccctggccatcagtgggctccagtctgaggatgagg
ctgattattactgtgcttcatgggatgacagcctgaatggccgttatgtcttcggaactgggaccaaggtcaccgtcctaggt
[SEQ ID NO: 193]
tctagaggtggtggtggtagcggcggcggcggctctggtggtggtggatccctcgagatggcc [SEQ ID NO: 305]
caggtccagctggtacagtctggggctgaggtgaggaagcctggggcctcagtgaaggtttcctgcaagacttctggat
acaccttcagttggtatgctatacattgggtgcgccaggcccccggacaaaggcttgagtggatgggatggatcaacgct
ggcaatggaaacacaaaatattcacagaaatttcagggcagagtcagtcttaccagggacacatccgcgagcacagcc
tacatggagctgagcagcctgagatctgatgacacggctgtgtattactgtgcgagacccgataattatggttcgggtgg
ggatgtttttgatatctggggccaagggacaatggtcaccgtctcttca [SEQ ID NO: 194]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSASGTPGQRVTISCSGSSSNIGSNTVNWYQQLPGTAPKLLIYSNDQRP
SGVPDRFSGSKSGTSASLAISGLQSEDEADYYCASWDDSLNGRYVFGTGTKVTV
LG [SEQ ID NO: 195]
SRGGGGSGGGGSGGGGSLEMA [SEQ ID NO: 307]
QVQLVQSGAEVRKPGASVKVSCKTSGYTFSWYAIHWVRQAPGQRLEWMG
WINAGNGNTKYSQKFQGRVSLTRDTSASTAYMELSSLRSDDTAVYYCARPD
NYGSGGDVFDIWGQGTMVTVSS [SEQ ID NO: 196]
TSGQAGQHHHHHHGAYPYDVPDYAS [SEQ ID NO: 308]

TABLE 50
ET200-100

DNA Sequence
(light chain variable region scFv linker heavy chain variable region His tag + HA tag)
aattttatgctgactcagccccactctgtgtcggagtctccggggaagacggtaaccatctcctgcacccgcagcagtggcagca
ttgccagcaactttgtgcagtggtaccagcagcgcccgggcagtgcccccacccctatgatctatgaggataacaacagacccc
ctggggtccctgatcggttctctgcctccgtcgacagctcctccaactctgcctccctcaccatctctggactgaagactgaggac
gaggctgactactactgtcagtcttatgataccagcaatgtggtattcggcggggggaccaagctgaccgtcctaggt [SEQ
ID NO: 197]
tctagaggtggtggtggtagcggcggcggcggctctggtggtggtggatccctcgagatggcc [SEQ ID NO: 305]
gaggtgcagctggtggagtctgggggaggcttggtacagcctggagggtccctgagactctcctgtgcagcctctggatt
caccttcagtagttatgaaatgaactgggtccgccaggctccagggaaggggctggagtgggtttcatacattagtagta
gtggtagtaccatatactacgcagactctgtgaagggccgattcaccatctccagagacaacgccaagaactcactgtat
ctgcaaatgaacagcctgagagccgaggacacggctgtttattactgtgcacgctgggactacggtatggacgtctggg
gccaagggaccacggtcaccgtctcctca [SEQ ID NO: 198]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
NFMLTQPHSVSESPGKTVTISCTRSSGSIASNFVQWYQQRPGSAPTPMIYEDNNRP
PGVPDRFSASVDSSSNSASLTISGLKTEDEADYYCQSYDTSNVVFGGGTKLTVLG
[SEQ ID NO: 199]
SRGGGGSGGGGSGGGGSLEMA [SEQ ID NO: 307]
EVQLVESGGGLVQPGGSLRLSCAASGFTFSSYEMNWVRQAPGKGLEWVSYI
SSSGSTIYYADSVKGRFTISRDNAKNSLYLQMNSLRAEDTAVYYCARWDYG
MDVWGQGTTVTVSS [SEQ ID NO: 200]
TSGQAGQHHHHHHGAYPYDVPDYAS [SEQ ID NO: 308]

TABLE 51
ET200-101

DNA Sequence
(light chain variable region scFv linker heavy chain variable region His tag +
HA tag)
caggctgtgctgactcagccaccctcagcgtctggggcccccgggcagagggtcaccgtctcttgttctggaagcaactccaac
atcggaagtaactacgttaactggtaccagcagttcccaggaacggcccccaaactcctcatgtatagtagtagtcagcggccct
caggggtccctgaccgattctctggctccaagtctggcacctcagcctccctggccatcagtgggctccactctgaggatgagg
ctgattattactgtgctacatgggatgacagcctgaatgcttgggtgttcggcggagggaccaagctgaccgtcctaggt [SEQ
ID NO: 201]
tctagaggtggtggtggtagcggcggcggcggctctggtggtggtggatccctcgagatggcc [SEQ ID NO: 305]
gaggtccagctggtgcagtctggggctgaggtgaggaagcctggggcctcagtgaaggtttcctgcaagacttctggat
acaccttcacttggtatgctatacattgggtgcgccaggcccccggacaaaggcttgagtggatgggatggatcaacgct
ggcagtggaaacacaaaatattcacagaaatttcagggcagagtcacccttaccagggacacatccgcgagcacagcg
tacatggagctgagcagcctgagatctgatgacacggctgtgtattactgtgcgagacccaataactatggttcgggtgg
ggatgtttttgatatctggggccaagggacaatggtcaccgtctcttca [SEQ ID NO: 202]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QAVLTQPPSASGAPGQRVTVSCSGSNSNIGSNYVNWYQQFPGTAPKLLMYSSSQ
RPSGVPDRFSGSKSGTSASLAISGLHSEDEADYYCATWDDSLNAWVFGGGTKLT
VLG [SEQ ID NO: 203]
SRGGGGSGGGGSGGGGSLEMA [SEQ ID NO: 307]
EVQLVQSGAEVRKPGASVKVSCKTSGYTFTWYAIHWVRQAPGQRLEWMG
WINAGSGNTKYSQKFQGRVTLTRDTSASTAYMELSSLRSDDTAVYYCARPN
NYGSGGDVFDIWGQGTMVTVSS [SEQ ID NO: 204]
TSGQAGQHHHHHHGAYPYDVPDYAS [SEQ ID NO: 308]

TABLE 52
ET200-102

DNA Sequence
(light chain variable region scFv linker heavy chain variable region His tag +
HA tag)
cagtctgtcgtgacgcagccgccctcagtgtctgcggccccaggacagaaggtcaccatctcctgctctggaagcagctccaa
cattgggaataattatgtatcctggtaccagcagctcccaggaacagcccccaaactcctcatttatgacaataataagcgaccct
cagggattcctgaccgattctctggctccaagtctggcacgtcagccaccctgggcatcaccggactccagactggggacgag
gccgattattactgcggaacatgggatagcagcctgagtgcttatgtcttcggaactgggaccaaggtcaccgtcctaggt
[SEQ ID NO: 205]
tctagaggtggtggtggtagcggcggcggcggctctggtggtggtggatccctcgagatggcc [SEQ ID NO: 305]
caggtccagctggtgcagtctggggctgaggtgaagaagcctggggcctcagtgaaagtttcctgcaaggcttctggat
acaccttcacgaactatgctctgcattgggtgcgccaggcccccggacaagggcttgagtggatggcatggatcaacgg
tggcaatggtaacacaaaatattcacagaacttccagggcagagtcaccattaccagggacacatccgcgagcacagc
ctatatggagctgagcagcctgagatctgaagacacggctgtgtattactgtgcgaaaccggaggaaacagctggaac
aatccactttgactactggggccagggaaccccggtcaccgtctcctca [SEQ ID NO: 206]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QSVVTQPPSVSAAPGQKVTISCSGSSSNIGNNYVSWYQQLPGTAPKLLIYDNNKR
PSGIPDRFSGSKSGTSATLGITGLQTGDEADYYCGTWDSSLSAYVFGTGTKVTVL
G [SEQ ID NO: 207]
SRGGGGSGGGGSGGGGSLEMA [SEQ ID NO: 307]
QVQLVQSGAEVKKPGASVKVSCKASGYTFTNYALHWVRQAPGQGLEWMA
WINGGNGNTKYSQNFQGRVTITRDTSASTAYMELSSLRSEDTAVYYCAKPEE
TAGTIHFDYWGQGTPVTVSS [SEQ ID NO: 208]
TSGQAGQHHHHHHGAYPYDVPDYAS [SEQ ID NO: 308]

TABLE 53
ET200-103

DNA Sequence
(light chain variable region scFv linker heavy chain variable region His tag +
HA tag)
caggctgtgctgactcagccccactctgtgtcggagtctccggggaagacggtaaccatctcctgcacccgcagcagtggcag
cattgccagcaactatgtgcagtggtaccagcagcgcccgggcagtgcccccaccactgtgatctatgaggataaccaaagac
cctctggggtccctgatcggttctctggctccatcgacagctcctccaactctgcctccctcaccatctctggactgaagactgag
gacgaggctgactactactgtcagtcttatgatagcaccatcacggtgttcggcggagggaccaagctgaccgtcctaggt
[SEQ ID NO: 209]
tctagaggtggtggtggtagcggcggcggcggctctggtggtggtggatccctcgagatggcc [SEQ ID NO: 305]
caggtccagctggtacagtctggggctgaggtgaagaagcctgggtcctcggtgaaggtctcctgcaaggcttctggag
gcaccttcagcagctatgctatcagctgggtgcgacaggcccctggacaagggcttgagtggatgggagggatcatccc
tatctttggtacagcaaactacgcacagaagttccagggcagagtcacgattaccgcggacgaatccacgagcacagc
ctacatggagctgagcagcctgagatctgaggacacggccgtgtattactgtgcgggggagggttactatgatagtagt
ggttattccaacggtgatgcttttgatatctggggccaagggacaatggtcaccgtctcttca [SEQ ID NO: 210]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QAVLTQPHSVSESPGKTVTISCTRSSGSIASNYVQWYQQRPGSAPTTVIYEDNQRP
SGVPDRFSGSIDSSSNSASLTISGLKTEDEADYYCQSYDSTITVFGGGTKLTVLG
[SEQ ID NO: 211]
SRGGGGSGGGGSGGGGSLEMA [SEQ ID NO: 307]
QVQLVQSGAEVKKPGSSVKVSCKASGGTFSSYAISWVRQAPGQGLEWMGGI
IPIFGTANYAQKFQGRVTITADESTSTAYMELSSLRSEDTAVYYCAGEGYYDS
SGYSNGDAFDIWGQGTMVTVSS [SEQ ID NO: 212]
TSGQAGQHHHHHHGAYPYDVPDYAS [SEQ ID NO: 308]

TABLE 54
ET200-104

DNA Sequence
(light chain variable region scFv linker heavy chain variable region His tag +
HA tag)
aattttatgctgactcagccccactctgtgtcggagtctccggggaagacggtaaccatctcctgcacccgcagcagtggcagca
ttgccagcaactatgtgcagtggtaccagcagcgcccgggcagtgcccccaccactgtgatctatgaggataaccaaagaccct
ctggggtccctgatcggttctctggctccatcgacagctcctccaactctgcctccctcaccatctctggactgaagactgaggac
gaggctgactactactgtcagtcttatgatagcagcaatgtggtattcggcggagggaccaaggtcaccgtcctaggt [SEQ
ID NO: 213]
tctagaggtggtggtggtagcggcggcggcggctctggtggtggtggatccctcgagatggcc [SEQ ID NO: 305]
gaggtgcagctggtggagtctgggggaggcttggtacagcctggagggtccctgagactctcctgtgcagcctctggatt
caccttcagtagttatgaaatgaactgggtccgccaggctccagggaaggggctggagtgggtttcatacattagtagta
gtggtagtaccatatactacgcagactctgtgaagggccgattcaccatctccagagacaacgccaagaactcactgtat
ctgcaaatgaacagcctgagagccgaggacacggctgtttattactgtgcacgctgggactacggtatggacgtctggg
gccaagggaccacggtcaccgtctcctca [SEQ ID NO: 214]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSSNVVFGGGTKVTVLG
[SEQ ID NO: 215]
SRGGGGSGGGGSGGGGSLEMA [SEQ ID NO: 307]
EVQLVESGGGLVQPGGSLRLSCAASGFTFSSYEMNWVRQAPGKGLEWVSYI
SSSGSTIYYADSVKGRFTISRDNAKNSLYLQMNSLRAEDTAVYYCARWDYG
MDVWGQGTTVTVSS [SEQ ID NO: 216]
TSGQAGQHHHHHHGAYPYDVPDYAS [SEQ ID NO: 308]

TABLE 55
ET200-105

DNA Sequence
(light chain variable region scFv linker heavy chain variable region His tag +
HA tag)
tcctatgtgctgactcagccaccctcagtgtccgtgtccccaggacagacagccagcatcacctgctctggagatagattgacga
ataaatatgtttcctggtatcaacagaagccaggccagtcccctgtgttggtcatctatgaggatgccaagcggccctcagggatc
cctgcgcgattctctggctccaactctgggaacacagccactctgaccatcagcgggacccaggctatggatgagtctgaatatt
actgtcaggcgtgggacagcagtgtggtggtttttggcggagggaccaagctgaccgtcctaggt [SEQ ID NO: 217]
tctagaggtggtggtggtagcggcggcggcggctctggtggtggtggatccctcgagatggcc [SEQ ID NO: 305]
gaggtgcagctggtggagtctgggggaggcttggtacagcctggcaggtccctgagactctcctgtgcagcctctggatt
tacctttgatgattatgccatgcactgggtccggcaagctccagggaagggcctggagtgggtctcaggtattagttgga
atagtggtagtataggctatgcggactctgtgaagggccgattcaccatctccagagacaacgccaagaactccctgtat
ctgcaaatgaacagtctgagagatgaggacacggccttgtattactgtgcaaaagaccgaggggggggagttatcgtta
aggatgcttttgatatctggggccaagggacaatggtcaccgtctcttca [SEQ ID NO: 218]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
SYVLTQPPSVSVSPGQTASITCSGDRLTNKYVSWYQQKPGQSPVLVIYEDAKRPS
GIPARFSGSNSGNTATLTISGTQAMDESEYYCQAWDSSVVVFGGGTKLTVLG
[SEQ ID NO: 219]
SRGGGGSGGGGSGGGGSLEMA [SEQ ID NO: 307]
EVQLVESGGGLVQPGRSLRLSCAASGFTFDDYAMHWVRQAPGKGLEWVSG
ISWNSGSIGYADSVKGRFTISRDNAKNSLYLQMNSLRDEDTALYYCAKDRGG
GVIVKDAFDIWGQGTMVTVSS [SEQ ID NO: 220]
TSGQAGQHHHHHHGAYPYDVPDYAS [SEQ ID NO: 308]

TABLE 56
ET200-106

DNA Sequence
(light chain variable region scFv linker heavy chain variable region His tag +
HA tag)
tcctatgagctgactcagccacccgcagcgtctgggacccccggacagagagtcaccatctcttgttctgggggcgtctccaac
atcgggagtggtgctctaaattggtaccagcaactcccaggaacggcccccaaactcctcatctatagttacaatcagcggccct
caggggtctctgaccgattctctggctccaggtctgccacctcagcctccctggccatcagtgggctccagtctgaggatgaggc
tgattattactgtgcaacctgggatgatagtgtgaatggttgggtgttcggcggagggaccaagctgaccgtcctaggt [SEQ
ID NO: 221]
tctagaggtggtggtggtagcggcggcggcggctctggtggtggtggatccctcgagatggcc [SEQ ID NO: 305]
gaggtgcagctggtggagtctggagctgaggtgaagaagcctggggattcagtgaaggtctcctgcaagccttctggtt
acaattttctcaactatggtatcaactgggtgcgacaggcccctggacaagggcttgagtggatgggatggattagcact
tacaccggtaacacaaactatgcacagaagctgcagggcagagtcaccttcaccacagacacatccacgagcacagcc
tacatggagatgaggagcctgagatctgacgacacggccgtgtattactgtgcgcgccagcagggtggtggttggtacg
atgtttggggtcaaggtactctggtcaccgtctcctca [SEQ ID NO: 222]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
SYELTQPPAASGTPGQRVTISCSGGVSNIGSGALNWYQQLPGTAPKLLIYSYNQR
PSGVSDRFSGSRSATSASLAISGLQSEDEADYYCATWDDSVNGWVFGGGTKLTV
LG [SEQ ID NO: 223]
SRGGGGSGGGGSGGGGSLEMA [SEQ ID NO: 307]
EVQLVESGAEVKKPGDSVKVSCKPSGYNFLNYGINWVRQAPGQGLEWMG
WISTYTGNTNYAQKLQGRVTFTTDTSTSTAYMEMRSLRSDDTAVYYCARQQ
GGGWYDVWGQGTLVTVSS [SEQ ID NO: 224]
TSGQAGQHHHHHHGAYPYDVPDYAS [SEQ ID NO: 308]

TABLE 57
ET200-107

DNA Sequence
(light chain variable region scFv linker heavy chain variable region His tag +
HA tag)
cagtctgtcgtgacgcagccgccctcagtgtctgcggccccaggagagaaggtcaccatctcctgctctggaagcaacttcaat
gttggaaataatgatgtatcctggtatcagcaactcccaggtgcagcccccaaactcctcatttatgacaataataagcgaccctca
gggattcctgaccgattctctggctccaagtctggcacgtcagccaccctggacatcaccgggctccacagtgacgacgaggc
cgattattactgcggaacatgggatagcagcctgaatactgggggggtcttcggaactgggaccaaggtcaccgtcctaggt
[SEQ ID NO: 225]
tctagaggtggtggtggtagcggcggcggcggctctggtggtggtggatccctcgagatggcc [SEQ ID NO: 305]
gaggtccagctggtgcagtctggagctgaggtgaagaagcctggggcctcagtgaaggtctcctgcaaggcttctggtt
acacctttaccagctatactatcagctgggtacgacaggcccctggacaagggcttgagtggatgggatggatcagcac
ttacaatggtctcacaaactatgcacagaacctccagggcagagtcaccatgactacagacacattcacgaccacagcc
tacatggagctgaggagcctcagatctgacgacacggccgtgtattactgtgtgagagaggggtcccccgactacggtg
acttcgcgtcctttgactactggggccagggaaccctggtcaccgtctcctca [SEQ ID NO: 226]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QSVVTQPPSVSAAPGEKVTISCSGSNFNVGNNDVSWYQQLPGAAPKLLIYDNNK
RPSGIPDRFSGSKSGTSATLDITGLHSDDEADYYCGTWDSSLNTGGVFGTGTKVT
VLG [SEQ ID NO: 227]
SRGGGGSGGGGSGGGGSLEMA [SEQ ID NO: 307]
EVQLVQSGAEVKKPGASVKVSCKASGYTFTSYTISWVRQAPGQGLEWMGW
ISTYNGLTNYAQNLQGRVTMTTDTFTTTAYMELRSLRSDDTAVYYCVREGS
PDYGDFASFDYWGQGTLVTVSS [SEQ ID NO: 228]
TSGQAGQHHHHHHGAYPYDVPDYAS [SEQ ID NO: 308]

TABLE 58
ET200-108

DNA Sequence
(light chain variable region scFv linker heavy chain variable region His tag +
HA tag)
cagtctgtgttgacgcagccgccctcagtgtctgcgcccccgggacagaaggtcaccatctcctgctctggaagcagctccaac
attgggaataattatgtatcctggtaccagcagttcccaggaacagcccccaaactcctcatttatgacaataataagcgaccctca
gggatttctgaccgattctctggctccaagtctggcacgtcagccaccctgggcatcgccggactccagactggggacgaggc
cgattattactgcggaacatgggataccagcctgagtggtttttatgtcttcggaagtgggaccaaggtcaccgtcctaggt
[SEQ ID NO: 229]
tctagaggtggtggtggtagcggcggcggcggctctggtggtggtggatccctcgagatggcc [SEQ ID NO: 305]
gaggtccagctggtacagtctggagctgaggtgaagaagcctggggcctcagtgaaggtctcctgcaaggcttctggtt
acacctttaccagctatactatcagctgggtacgacaggcccctggacaagggcttgagtggatgggatggatcagcac
ttacaatggtctcacaaactatgcacagaacctccagggcagagtcaccatgactacagacacattcacgaccacagcc
tacatggagctgaggagcctcagatctgacgacacggccgtgtattactgtgtgagagaggggtcccccgactacggtg
acttcgcgtcctttgactactggggccagggaaccctggtcaccgtctcctca [SEQ ID NO: 230]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QSVLTQPPSVSAPPGQKVTISCSGSSSNIGNNYVSWYQQFPGTAPKLLIYDNNKRP
SGISDRFSGSKSGTSATLGIAGLQTGDEADYYCGTWDTSLSGFYVFGSGTKVTVL
G [SEQ ID NO: 231]
SRGGGGSGGGGSGGGGSLEMA [SEQ ID NO: 307]
EVQLVQSGAEVKKPGASVKVSCKASGYTFTSYTISWVRQAPGQGLEWMGW
ISTYNGLTNYAQNLQGRVTMTTDTFTTTAYMELRSLRSDDTAVYYCVREGS
PDYGDFASFDYWGQGTLVTVSS [SEQ ID NO: 232]
TSGQAGQHHHHHHGAYPYDVPDYAS [SEQ ID NO: 308]

TABLE 59
ET200-109

DNA Sequence
(light chain variable region scFv linker heavy chain variable region His tag +
HA tag)
ctgcctgtgctgactcagccaccctcagcgtctgcgacccccgggcagagggtcaccatctcttgttctggaaccacctccaaca
tcggaagtaatactgtacactggtaccagcagctcccagggacggcccccaaactcctcatctataataataatcagcggccctc
aggggtccctgaccgattctctggctccaagtctggcacctcagcctccctggccatcagtgggctccggtccgaggatgaggc
tacatattcctgtgcaacatgggatgacagcctgagtggtgtggtcttcggcggagggaccaagctgaccgtcctaggt [SEQ
ID NO: 233]
tctagaggtggtggtggtagcggcggcggcggctctggtggtggtggatccctcgagatggcc [SEQ ID NO: 305]
gaggtccagctggtgcagtctggggctgaggtgaagaagcctgggtcctcggtgaaggtctcctgcaaggcttctggag
gcaccttcagcagctatgctatcagctgggtgcgacaggcccctggacaagggcttgagtggatgggagggatcatccc
tatctttggtacagcaaactacgcacagaagttccagggcagagtcacgattaccgcggacgaatccacgagcacagc
ctacatggagctgagcagcctgagatctgaggacacggccgtgtattactgtgcgagagatcccgcctacggtgactac
gagtatgatgcttttgatatctggggccaagggacaatggtcaccgtctcttca [SEQ ID NO: 234]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
LPVLTQPPSASATPGQRVTISCSGTTSNIGSNTVHWYQQLPGTAPKLLIYNNNQRP
SGVPDRFSGSKSGTSASLAISGLRSEDEATYSCATWDDSLSGVVFGGGTKLTVLG
[SEQ ID NO: 235]
SRGGGGSGGGGSGGGGSLEMA [SEQ ID NO: 307]
EVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARDPAYGD
YEYDAFDIWGQGTMVTVSS [SEQ ID NO: 236]
TSGQAGQHHHHHHGAYPYDVPDYAS [SEQ ID NO: 308]

TABLE 60
ET200-110

DNA Sequence
(light chain variable region scFv linker heavy chain variable region His tag +
HA tag)
cagtctgtgttgacgcagccgccctcagcgtctgggacccccgggcagagggtcaccatctcttgttctggaagcagctccaac
atcggaactaatggtgtaaactggttccagcagttcccaggaacggcccccaaactcctcatctatactaatgatcagcggccctc
aggggtccctgaccgattctctggctccaagtctggcacctcagcctccctggccatcagtgggctccagtctgcggatgaggct
gattattactgtgcagtgtgggaccacagcctgaatggtccggtgttcggcggagggaccaagctgaccgtcctaggt [SEQ
ID NO: 237]
tctagaggtggtggtggtagcggcggcggcggctctggtggtggtggatccctcgagatggcc [SEQ ID NO: 305]
caggtgcagctggtgcagtctggggctgaggtgaagaagcctgggtcctcggtgaaggtctcctgcaaggcttctggag
gcaccttcagcagctatgctatcagctgggtgcgacaggcccctggacaagggcttgagtggatgggagggatcatccc
tatctttggtacagcaaactacgcacagaagttccagggcagagtcacgattaccgcggacgaatccacgagcacagc
ctacatggagctgagcagcctgagatctgaggacacggccgtgtattactgtgcgagaggggccggttttgatgcttttg
atatctggggccaagggacaatggtcaccgtctcttca [SEQ ID NO: 238]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QSVLTQPPSASGTPGQRVTISCSGSSSNIGTNGVNWFQQFPGTAPKLLIYTNDQRP
SGVPDRFSGSKSGTSASLAISGLQSADEADYYCAVWDHSLNGPVFGGGTKLTVL
G [SEQ ID NO: 239]
SRGGGGSGGGGSGGGGSLEMA [SEQ ID NO: 307]
QVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARGAGFD
AFDIWGQGTMVTVSS [SEQ ID NO: 240]
TSGQAGQHHHHHHGAYPYDVPDYAS [SEQ ID NO: 308]

TABLE 61
ET200-111

DNA Sequence
(light chain variable region scFv linker heavy chain variable region His tag +
HA tag)
caggctgtgctgactcagccaccctcagcgtctgggacccccgggcagagggtcaccatctcttgttctggaagcagctccaac
atcggaagtaatactgtaaactggtaccagcagctcccaggaacggcccccaaactcctcatctatagtaataatcagcggccct
caggggtccctgaccgattctctggctccaagtctggcacctcagcctccctggccatcagtgggctccagtctgaggatgaga
ctgattattactgtgcagcatgggatgacagcctgaatggttatgtcttcggaactgggaccaaggtcaccgtcctaggt [SEQ
ID NO: 241]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccctcacctgcgctgtctatggtgg
gtccttcagtggttactactggagctggatccgccagcccccagggaaggggctggagtggattggggaaatcaatcat
agtggaagcaccaactacaacccgtccctcaagagtcgagtcaccatatcagtagacacgtccaagaaccagttctccc
tgaagctgagctctgtgaccgccgcggacacggctgtgtattactgtgcgagagaggggctagatgcttttgatatctgg
ggccaagggacaatggtcaccgtctcttca [SEQ ID NO: 242]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QAVLTQPPSASGTPGQRVTISCSGSSSNIGSNTVNWYQQLPGTAPKLLIYSNNQRP
SGVPDRFSGSKSGTSASLAISGLQSEDETDYYCAAWDDSLNGYVFGTGTKVTVL
G [SEQ ID NO: 243]
SRGGGGSGGGGSGGGGSLEMA [SEQ ID NO: 307]
QVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCAREGLDAFD
IWGQGTMVTVSS [SEQ ID NO: 244]
TSGQAGQHHHHHHGAYPYDVPDYAS [SEQ ID NO: 308]

TABLE 62
ET200-112

DNA Sequence
(light chain variable region scFv linker heavy chain variable region His tag +
HA tag)
caggctgtgctgactcagccaccctcagcgtctgggacccccgggcagagggtcaccatctcttgttctggaagcagctccaac
atcggaagtaatactgtaaactggtaccagcagctcccaggaacggcccccaaactcctcatgtatagtaatgatcagcggccct
caggggtccctgaccgattctctggctccaagtctggcacctcagcctccctggccatcagtgggctccagtctgaggatgagg
ctgattattattgtgcagcatgggatgacagcctgaatggttatgtcttcgcagctgggacccagctcaccgttttaagt [SEQ
ID NO: 245]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccctcacctgcgctgtctatggtgg
gtccttcagtggttactactggagctggatccgccagcccccagggaaggggctggagtggattggggaaatcaatcat
agtggaagcaccaactacaacccgtccctcaagagtcgagtcaccatatcagtagacacgtccaagaaccagttctccc
tgaagctgagctctgtgaccgccgcggacacggctgtgtattactgtgcgagagaggggctagatgcttttgatatctgg
ggccaagggacaatggtcaccgtctcttca [SEQ ID NO: 246]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QAVLTQPPSASGTPGQRVTISCSGSSSNIGSNTVNWYQQLPGTAPKLLMYSNDQR
PSGVPDRFSGSKSGTSASLAISGLQSEDEADYYCAAWDDSLNGYVFAAGTQLTV
LS [SEQ ID NO: 247]
SRGGGGSGGGGSGGGGSLEMA [SEQ ID NO: 307]
QVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCAREGLDAFD
IWGQGTMVTVSS [SEQ ID NO: 248]
TSGQAGQHHHHHHGAYPYDVPDYAS [SEQ ID NO: 308]

TABLE 63
ET200-113

DNA Sequence
(light chain variable region scFv linker heavy chain variable region His tag +
HA tag)
cagtctgtcgtgacgcagccgccctcagtgtctgcggccccaggacagaaggtcaccatctcctgctctggaagcagctccaa
cattgggaataattatgtatcctggtaccagcagctcccaggaacagcccccaaactcctcatttatgacaataataagcgaccct
cagggattcctgaccgattctctggctccaagtctggcacgtcagccaccctgggcatcactggactccagactggggacgag
gccgattattactgcggaacatgggatagcagcctgagtgctgcttatgtcttcggaactgggaccaaggtcaccgtcctaggt
[SEQ ID NO: 249]
tctagaggtggtggtggtagcggcggcggcggctctggtggtggtggatccctcgagatggcc [SEQ ID NO: 305]
caggtccagctggtacagtctggagctgaggtgaagaagcctggggcctcagtgaaggtctcctgcaaggcttctggtta
cagctttaccagctatactatcagctgggttcgacaggcccctggacaaggccttgagtggatgggatgggtcagcactt
acaatggtctcagaaactatgcacagaacctccagggcagagtcaccatgactacagacacactcacgaccacagcct
acatggagctgaggagcctcagatctgacgacacggccgtgtattattgtgtgagagaggggtcccccgactacggtga
cttcgcggcctttgactactggggccagggcaccctggtcaccgtctcctca [SEQ ID NO: 250]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QSVVTQPPSVSAAPGQKVTISCSGSSSNIGNNYVSWYQQLPGTAPKLLIYDNNKR
PSGIPDRFSGSKSGTSATLGITGLQTGDEADYYCGTWDSSLSAAYVFGTGTKVTV
LG [SEQ ID NO: 251]
SRGGGGSGGGGSGGGGSLEMA [SEQ ID NO: 307]
QVQLVQSGAEVKKPGASVKVSCKASGYSFTSYTISWVRQAPGQGLEWMGW
VSTYNGLRNYAQNLQGRVTMTTDTLTTTAYMELRSLRSDDTAVYYCVREG
SPDYGDFAAFDYWGQGTLVTVSS [SEQ ID NO: 252]
TSGQAGQHHHHHHGAYPYDVPDYAS [SEQ ID NO: 308]

TABLE 64
ET200-114

DNA Sequence
(light chain variable region scFv linker heavy chain variable region His tag +
HA tag)
caggctgtgctgactcagccaccctcagcgtctgagacccccgggcagagggtcaccatctcttgttctggaagcaggtccaac
atcggaactaatattgtacactggtaccagcagcgcccaggaatggcccccaaactcctcacttatggtagtcggcggccctca
ggggtcccggaccgattctctggctccaagtttggcacctcagcctccctggccatcagtgggctccagtctgaggatgaggct
gattattattgtgcagcatgggatgacagtctgaatggtccggctttcggcggagggaccaagctgaccgtcctaggt [SEQ
ID NO: 253]
tctagaggtggtggtggtagcggcggcggcggctctggtggtggtggatccctcgagatggcc [SEQ ID NO: 305]
caggtgcagctacagcagtggggcgcaggactgttgaagccttcggagaccctgtccctcacctgcgctgtctatggtgg
gtccttcagtggttactactggagctggatccgccagcccccagggaaggggctggagtggattggggaaatcaatcat
agtggaagcaccaactacaacccgtccctcaagagtcgagtcaccatatcagtagacacgtccaagaaccagttctccc
tgaagctgagctctgtgaccgccgcggacacggctgtgtattactgtgcgagagacggtgggggctactttgactactgg
ggccagggaaccctggtcaccgtctcctca [SEQ ID NO: 254]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QAVLTQPPSASETPGQRVTISCSGSRSNIGTNIVHWYQQRPGMAPKLLTYGSRRP
SGVPDRFSGSKFGTSASLAISGLQSEDEADYYCAAWDDSLNGPAFGGGTKLTVL
G [SEQ ID NO: 255]
SRGGGGSGGGGSGGGGSLEMA [SEQ ID NO: 307]
QVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCARDGGGYF
DYWGQGTLVTVSS [SEQ ID NO: 256]
TSGQAGQHHHHHHGAYPYDVPDYAS [SEQ ID NO: 308]

TABLE 65
ET200-115

DNA Sequence
(light chain variable region scFv linker heavy chain variable region His tag +
HA tag)
cagtctgtgttgacgcagccgccctcagtgtctggggccccagggcagagggtcaccatctcctgcactgggagcagctccaa
tatcggggcacgttatgatgtacactggtaccagcaactcccaggaacagccccccgactcctcatctctgctaactacgatcgg
ccctcaggggtccctgaccgattctctggctccaagtctggcacctcagcctccctggccatcactgggctccaggctgaggat
gaggctgattattactgccagtcctatgacagcagtgtgagtgcttgggtgttcggcggagggaccaaggtcaccgtcctaggt
[SEQ ID NO: 257]
tctagaggtggtggtggtagcggcggcggcggctctggtggtggtggatccctcgagatggcc [SEQ ID NO: 305]
gaagtgcagctggtgcagtctggggctgaagtgaaggagcctggggcctcagtgaggatctcctgccaggcatctggat
acaacttcatcagttattatatgcactgggtgcggcaggcccctgggcaaggtcttgagtggatgggcaccatcaaccca
ggcagtggtgagacagactactcacagaagttgcagggcagagtcaccatgaccagggacccgtccacgggtacattc
gacatggggctgagcagcctgacatctggggacacggccgtctattattgtgcgacaggtctcatcagaggagctagcg
atgcttttaatatctggggccgggggacaatggtcaccgtctcttca [SEQ ID NO: 258]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy
chain variable region His tag + HA tag)
QSVLTQPPSVSGAPGQRVTISCTGSSSNIGARYDVHWYQQLPGTAPRLLISANYD
RPSGVPDRFSGSKSGTSASLAITGLQAEDEADYYCQSYDSSVSAWVFGGGTKVT
VLG [SEQ ID NO: 259]
SRGGGGSGGGGSGGGGSLEMA [SEQ ID NO: 307]
EVQLVQSGAEVKEPGASVRISCQASGYNFISYYMHWVRQAPGQGLEWMGT
INPGSGETDYSQKLQGRVTMTRDPSTGTFDMGLSSLTSGDTAVYYCATGLIR
GASDAFNIWGRGTMVTVSS [SEQ ID NO: 260]
TSGQAGQHHHHHHGAYPYDVPDYAS [SEQ ID NO: 308]

TABLE 66
ET200-116

DNA Sequence
(light chain variable region scFv linker heavy chain variable region His tag + HA tag)
cagcctgtgctgactcagccaccctcagtgtccgtgtccccaggacagacggccgccatcccctgttctggagataagttgggg
gataaatttgcttcctggtatcagcagaagccaggccagtcccctgtgctggtcatctatcaagatactaagcggccctcagggat
ccctgagcgattctctggctccaactctgggaacacagccactctgaccatcagcgggacccaggctatggatgaggctgacta
ttactgtcagacgtgggccagcggcattgtggtgttcggcggagggaccaagctgaccgtcctaggt [SEQ ID NO:
261]
tctagaggtggtggtggtagcggcggcggcggctctggtggtggtggatccctcgagatggcc [SEQ ID NO: 305]
caggtacagctgcagcagtcaggtccaggactggtgaagccctcgcagaccctctcactcacctgtgccatctccgggg
acagtgtctctagcaacagtgctgcttggaactggatcaggcagtccccatcgagaggccttgagtggctgggaaggac
atactacaggtccaagtggtataatgattatgcagtatctgtgaaaagtcgaataaccatcaacccagacacatccaag
aaccagttctccctgcagctgaactctgtgactcccgaggacacggctgtgtattactgtgcaagagagcgcagtggctg
gaagggatttgactactggggccagggaaccctggtcaccgtctcctca [SEQ ID NO: 262]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QPVLTQPPSVSVSPGQTAAIPCSGDKLGDKFASWYQQKPGQSPVLVIYQDTKRPS
GIPERFSGSNSGNTATLTISGTQAMDEADYYCQTWASGIVVFGGGTKLTVLG
[SEQ ID NO: 263]
SRGGGGSGGGGSGGGGSLEMA [SEQ ID NO: 307]
QVQLQQSGPGLVKPSQTLSLTCAISGDSVSSNSAAWNWIRQSPSRGLEWLGR
TYYRSKWYNDYAVSVKSRITINPDTSKNQFSLQLNSVTPEDTAVYYCARERS
GWKGFDYWGQGTLVTVSS [SEQ ID NO: 264]
TSGQAGQHHHHHHGAYPYDVPDYAS [SEQ ID NO: 308]

TABLE 67
ET200-117

DNA Sequence
(light chain variable region scFv linker heavy chain variable region His tag + HA tag)
gatgttgtgatgactcagtctccaccctccctgtccgtcacccctggagagccggcctccatcacctgcaggtctagtcagagcct
cctggaaagaaatgcatacaactacttggattggtacctgcagaggccaggacagtctccacagctcctgatctacttgggttcta
atcgggccgccggggtccctgacaggttcagtggcagtggatcaggcagagattttacactgaaaatcagcagagtggagcct
gaggatgttggggtttattactgcatgcaagctctacaagctccgttcactttcggcggagggaccaaggtggagatcaaacgt
[SEQ ID NO: 265]
tctagaggtggtggtggtagcggcggcggcggctctggtggtggtggatccctcgagatggcc [SEQ ID NO: 305]
gaagtgcagctggtgcagtctgggggaggcttggtacagcctggggggtccctgagactctcctgtgcagcctctggatt
cacctttagcagctatgccatgagctgggtccgccaggctccagggaaggggctggagtgggtctcagctattagtggta
gtggtggtagcacatactacgcagactccgtgaagggccggttcaccatctccagagacaattccaagaacacgctgta
tctgcaaatgaacagcctgagagccgaggacacggccgtatattactgtgcgaaatggggcccgtttcaggatgcttttg
atatctggggccaagggacaatggtcaccgtctcttca [SEQ ID NO: 266]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
DVVMTQSPPSLSVTPGEPASITCRSSQSLLERNAYNYLDWYLQRPGQSPQLLIYL
GSNRAAGVPDRFSGSGSGRDFTLKISRVEPEDVGVYYCMQALQAPFTFGGGTKV
EIKR [SEQ ID NO: 267]
SRGGGGSGGGGSGGGGSLEMA [SEQ ID NO: 307]
EVQLVQSGGGLVQPGGSLRLSCAASGFTFSSYAMSWVRQAPGKGLEWVSAI
SGSGGSTYYADSVKGRFTISRDNSKNTLYLQMNSLRAEDTAVYYCAKWGPF
QDAFDIWGQGTMVTVSS [SEQ ID NO: 268]
TSGQAGQHHHHHHGAYPYDVPDYAS [SEQ ID NO: 308]

TABLE 68
ET200-118

DNA Sequence
(light chain variable region scFv linker heavy chain variable region His tag + HA tag)
caggctgtgctgactcagcctgcctccgtgtctgggtctcctggacagtcgatcaccatctcctgcactggaaccagcagtgacg
ttggtggttataactatgtctcctggtaccaacagcacccgggcaaagcccccaaactcatgatttatgaggtcagtaatcggccc
tcaggggtttctaatcgcttctctggctccaagtctggcaacacggcctccctgaccatctctgggctccaggctgaggacgagg
ctgattattactgcagctcatatacaagcagcagcaccccttatgtcttcggagcagggaccaaggtcaccgtcctaggt [SEQ
ID NO: 269]
tctagaggtggtggtggtagcggcggcggcggctctggtggtggtggatccctcgagatggcc [SEQ ID NO: 305]
gaggtgcagctggtggagtctgggggaggcttggtacagcctggcaggtccctgagactctcctgtgcagcctctggatt
cacctttgatgattatgccatgcactgggtccggcaagctccagggaagggcctggagtgggtctcaggtattagttgga
atagtggtagcataggctatgcggactctgtgaagggccgattcaccatctccagagacaacgccaagaactccctgtat
ctgcaaatgaacagtctgagagctgaggacacggccttgtattactgtgcaaaagccaggtggacagcagtggcatca
gaccaccactttgactactggggccagggaacgctggtcaccgtctcctca [SEQ ID NO: 270]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QAVLTQPASVSGSPGQSITISCTGTSSDVGGYNYVSWYQQHPGKAPKLMIYEVSN
RPSGVSNRFSGSKSGNTASLTISGLQAEDEADYYCSSYTSSSTPYVFGAGTKVTV
LG [SEQ ID NO: 271]
SRGGGGSGGGGSGGGGSLEMA [SEQ ID NO: 307]
EVQLVESGGGLVQPGRSLRLSCAASGFTFDDYAMHWVRQAPGKGLEWVSG
ISWNSGSIGYADSVKGRFTISRDNAKNSLYLQMNSLRAEDTALYYCAKARW
TAVASDHHFDYWGQGTLVTVSS [SEQ ID NO: 272]
TSGQAGQHHHHHHGAYPYDVPDYAS [SEQ ID NO: 308]

TABLE 69
ET200-119

DNA Sequence
(light chain variable region scFv linker heavy chain variable region His tag + HA tag)
caggctgtgcttactcagccaccctcagcgtctgggacccccgggcagagggtcaccatctcttgttctggaagcagctccaac
atcggaagtaatactgtaaactggtaccagcagctcccaggaacggcccccaaactcctcatctatagtaataatcagcggccct
caggggtccctgaccgattctctggctccaagtctggcacctcagcctccctggccatcagtgggctccagtctgaggatgagg
ctgattattactgtgcagcatgggatgacagcctgaatggttatgtcttcggaactgggaccaagctgaccgtcctaggt [SEQ
ID NO: 273]
tctagaggtggtggtggtagcggcggcggcggctctggtggtggtggatccctcgagatggcc [SEQ ID NO: 305]
gaggtgcagctggtgcagtctggggctgaggtgaagaagcctgggtcctcggtgaaggtctcctgcaaggcttctggag
gcaccttcagcagctatgctatcagctgggtgcgacaggcccctggacaagggcttgagtggatgggagggatcatccc
tatctttggtacagcaaactacgcacagaagttccagggcagagtcacgattaccgcggacgaatccacgagcacagc
ctacatggagctgagcagcctgagatctgaggacacggccgtgtattactgtgcgagagattgggactacatggacgtc
tggggcaaagggaccacggtcaccgtctcctca [SEQ ID NO: 274]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QAVLTQPPSASGTPGQRVTISCSGSSSNIGSNTVNWYQQLPGTAPKLLIYSNNQRP
SGVPDRFSGSKSGTSASLAISGLQSEDEADYYCAAWDDSLNGYVFGTGTKLTVL
G [SEQ ID NO: 275]
SRGGGGSGGGGSGGGGSLEMA [SEQ ID NO: 307]
EVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARDWDYM
DVWGKGTTVTVSS [SEQ ID NO: 276]
TSGQAGQHHHHHHGAYPYDVPDYAS [SEQ ID NO: 308]

TABLE 70
ET200-120

DNA Sequence
(light chain variable region scFv linker heavy chain variable region His tag + HA tag)
tcctatgagctgactcagccaccctcagcgtctgggacccccgggcagagggtcaccatctcttgttctggaagcagctccaac
atcggaagtaatactgtaaactggtaccagcagctcccaggaacggcccccaaactcctcatctatagtaataatcagcggccct
caggggtccctgaccgattctctggctccaagtctggcacctcagcctccctggccatcagtgggctccagtctgaggatgagg
ctgattattactgtgcagcatgggatgacagcctgaatggttatgtcttcggaactgggaccaaggtcaccgtcctaggt [SEQ
ID NO: 277]
tctagaggtggtggtggtagcggcggcggcggctctggtggtggtggatccctcgagatggcc [SEQ ID NO: 305]
gaggtgcagctggtggagtctggagctgaggtgaagaagcctggggcctcagtgaaggtctcctgcaaggcttctggtt
acacctttaccagctatggtatcagctgggtgcgacaggcccctggacaagggcttgagtggatgggatggatcagcgc
ttacaatggtaacacaaactatgcacagaagctccagggcagagtcaccatgaccacagacacatccacgagcacagc
ctacatggagctgaggagcctgagatctgacgacacggccgtgtattactgtgcgagagacctatctcggggagctaac
ccgcattactactactactacggtatggacgtctggggccaagggaccacggtcaccgtctcctca [SEQ ID NO:
278]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SYELTQPPSASGTPGQRVTISCSGSSSNIGSNTVNWYQQLPGTAPKLLIYSNNQRP
SGVPDRFSGSKSGTSASLAISGLQSEDEADYYCAAWDDSLNGYVFGTGTKVTVL
G [SEQ ID NO: 279]
SRGGGGSGGGGSGGGGSLEMA [SEQ ID NO: 307]
EVQLVESGAEVKKPGASVKVSCKASGYTFTSYGISWVRQAPGQGLEWMGW
ISAYNGNTNYAQKLQGRVTMTTDTSTSTAYMELRSLRSDDTAVYYCARDLS
RGANPHYYYYYGMDVWGQGTTVTVSS [SEQ ID NO: 280]
TSGQAGQHHHHHHGAYPYDVPDYAS [SEQ ID NO: 308]

TABLE 71
ET200-121

DNA Sequence
(light chain variable region scFv linker heavy chain variable region His tag + HA tag)
cagtctgtgttgacgcagccgccctcagtgtctggggccccagggcagagggtcaccgtctcctgcactgggagcagatccaa
catcggggcaggatatgatgtacactggtaccagcaacttccaggaacagcccccaaactcctcatctatggaaatagtaatcgg
cctccaggggtccctgaccgattctctgggtctaagtctggcacctcagcctccctggtcatcactgggctccaggctgaggatg
ccgctgattattactgccagtcctatgacaacactgtgcgtgaatcaccttatgtcttcggaactgggaccaaggtcaccgtcctag
gt [SEQ ID NO: 281]
tctagaggtggtggtggtagcggcggcggcggctctggtggtggtggatccctcgagatggcc [SEQ ID NO: 305]
gaggtccagctggtacagtctggggctgaggtgaagaagcctggggcctcagtgaaggtctcctgcaaggtttccggat
acaccctcactgaattatccatgcactgggtgcgacaggctcctggaaaagggcttgagtggatgggaggttttgatcct
gaagatggtgaaacaatctacgcacagaagttccagggcagagtcaccatgaccgaggacacatctacagacacagc
ctacatggagctgagcagcctgagatctgaggacacggccgtgtattactgtgcaacagagagtaatttagtgtcccggc
actactactactacggtatggacgtctggggccaagggaccacggtcaccgtctcctca [SEQ ID NO: 282]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QSVLTQPPSVSGAPGQRVTVSCTGSRSNIGAGYDVHWYQQLPGTAPKLLIYGNS
NRPPGVPDRFSGSKSGTSASLVITGLQAEDAADYYCQSYDNTVRESPYVFGTGTK
VTVLG [SEQ ID NO: 283]
SRGGGGSGGGGSGGGGSLEMA [SEQ ID NO: 307]
EVQLVQSGAEVKKPGASVKVSCKVSGYTLTELSMHWVRQAPGKGLEWMG
GFDPEDGETIYAQKFQGRVTMTEDTSTDTAYMELSSLRSEDTAVYYCATESN
LVSRHYYYYGMDVWGQGTTVTVSS [SEQ ID NO: 284]
TSGQAGQHHHHHHGAYPYDVPDYAS [SEQ ID NO: 308]

TABLE 72
ET200-122

DNA Sequence
(light chain variable region scFv linker heavy chain variable region His tag + HA tag)
ctgcctgtgctgactcagccaccctcagcgtctgggacccccgggcagagggtcaccatctcttgttctggaaccagctccaac
atcggaagtaattctgtagactggtaccagcagctcccaggaacggcccccaaactcctcatctatagtaataatcagcggccct
caggggtccctgaccgaatctctggctccaagtctggcacctcagcctccctggccatcagtgggctccagtctgaggatgagg
ctgattattactgtgcagcatgggatgacagcctgaatggttatgtcttcggaactgggaccaaggtcaccgtcctaggt [SEQ
ID NO: 285]
tctagaggtggtggtggtagcggcggcggcggctctggtggtggtggatccctcgagatggcc [SEQ ID NO: 305]
gaagtgcagctggtgcagtctggggctgaggtgaagaagcctggggcctcagtgaaggtctcctgcaaggcttctggat
acaccttcaccggctactatatgcactgggtgcgacaggcccctggacaagggcttgagtggatgggatggatcaaccc
taacagtggtggcacaaactatgcacagaagtttcagggcagggtcaccatgaccagggacacgtccatcagcacagc
ctacatggagctgagcaggctgagatctgacgacacggccgtgtattactgtgcgagagattacggatactatggttcgg
ggagttattcgagcggccccctttactactactacggtatggacgtctggggccaagggaccacggtcaccgtctcctca
[SEQ ID NO: 286]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
LPVLTQPPSASGTPGQRVTISCSGTSSNIGSNSVDWYQQLPGTAPKLLIYSNNQRP
SGVPDRISGSKSGTSASLAISGLQSEDEADYYCAAWDDSLNGYVFGTGTKVTVL
G [SEQ ID NO: 287]
SRGGGGSGGGGSGGGGSLEMA [SEQ ID NO: 307]
EVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCARDY
GYYGSGSYSSGPLYYYYGMDVWGQGTTVTVSS [SEQ ID NO: 288]
TSGQAGQHHHHHHGAYPYDVPDYAS [SEQ ID NO: 308]

TABLE 73
ET200-123

DNA Sequence
(light chain variable region scFv linker heavy chain variable region His tag + HA tag)
caggctgtgctgactcagccaccctcagcgtctgggacccccgggcagagggtcaccatctcttgttctggaagcagctccaac
atcggaagtaatactgtaaactggtaccagcagctcccaggaacggcccccaaactcctcatgtataataatgatcagcggccct
caggggtccctgaccgattctctggctccaagtctggcacctcagcctccctggccatcagtgggctccagtctgaggatgagg
ctgattattactgtgcagcatgggatgacagcctcaatggttatgtcttcggacctgggaccaaggtcaccgtcctaggt [SEQ
ID NO: 289]
tctagaggtggtggtggtagcggcggcggcggctctggtggtggtggatccctcgagatggcc [SEQ ID NO: 305]
caggtgcagctggtggagtctggagctgaggtgaagaagcctggggcctcagtgaaggtctcctgcaaggcttctggtt
acacctttaccagctatggtatcagctgggtgcgacaggcccctggacaagggcttgagtggatgggatggatcagcgc
ttacaatggtaacacaaactatgcacagaagctccagggcagagtcaccatgaccacagacacatccacgagcacagc
ctacatggagctgaggagcctgagatctgacgacacggccgtgtattactgtgcgagagacctatctcggggagctaac
ccgcattactactactactacggtatggacgtctggggccaagggaccacggtcaccgtctcctca [SEQ ID NO:
290]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QAVLTQPPSASGTPGQRVTISCSGSSSNIGSNTVNWYQQLPGTAPKLLMYNNDQ
RPSGVPDRFSGSKSGTSASLAISGLQSEDEADYYCAAWDDSLNGYVFGPGTKVT
VLG [SEQ ID NO: 291]
SRGGGGSGGGGSGGGGSLEMA [SEQ ID NO: 307]
QVQLVESGAEVKKPGASVKVSCKASGYTFTSYGISWVRQAPGQGLEWMGW
ISAYNGNTNYAQKLQGRVTMTTDTSTSTAYMELRSLRSDDTAVYYCARDLS
RGANPHYYYYYGMDVWGQGTTVTVSS [SEQ ID NO: 292]
TSGQAGQHHHHHHGAYPYDVPDYAS [SEQ ID NO: 308]

TABLE 74
ET200-125

DNA Sequence
(light chain variable region scFv linker heavy chain variable region His tag + HA tag)
aattttatgctgactcagccccacgctgtgtcggagtctccggggaagacggtaaccatctcctgcacccgcagcagtggcagt
attgccagcaactatgtgcagtggtaccagcagcgcccgggcagttccccccgcactgtgatttatgaggataatcaaagaccct
ctggggtccctggtcggttctctggctccatcgacagctcctccaactctgcctccctcaccatctctggactgaagactgaggac
gaggctgactactactgtcagtcttatgattccaccagtgtgcttttcggcggagggaccaagctgaccgtcctaggt [SEQ
ID NO: 293]
tctagaggtggtggtggtagcggcggcggcggctctggtggtggtggatccctcgagatggcc [SEQ ID NO: 305]
gaggtccagctggtgcagtctggggctgaggtgaagaagccagggtcctcggtgaaggtctcctgcaaggcctcggga
ggcaccttcagcagcaattctctcagctgggtgcgacaggcccctggacaagggcttgagtggatgggaaggatcttcc
ctatcctgggtataacaaactatgcacagaagttccagggcagagtcacgattaccgcggacaaatccacgagcacag
cctacatggagctgagcagcctgagatctgaggacacggccgtctattactgtgcgagaggaaactaccaatggtatga
tgcttttgatatctggggccaagggacaatggtcaccgtctcttca [SEQ ID NO: 294]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
NFMLTQPHAVSESPGKTVTISCTRSSGSIASNYVQWYQQRPGSSPRTVIYEDNQR
PSGVPGRFSGSIDSSSNSASLTISGLKTEDEADYYCQSYDSTSVLFGGGTKLTVLG
[SEQ ID NO: 295]
SRGGGGSGGGGSGGGGSLEMA [SEQ ID NO: 307]
EVQLVQSGAEVKKPGSSVKVSCKASGGTFSSNSLSWVRQAPGQGLEWMGRI
FPILGITNYAQKFQGRVTITADKSTSTAYMELSSLRSEDTAVYYCARGNYQW
YDAFDIWGQGTMVTVSS [SEQ ID NO: 296]
TSGQAGQHHHHHHGAYPYDVPDYAS [SEQ ID NO: 308]

TABLE 75
ET200-005

DNA Sequence
(light chain variable region scFv linker heavy chain variable region His tag + HA tag)
cagcctgtgctgactcagccaccctcagtgtcagtggtcccaggaaagacggccaggattacctgtgggggaaaaaacattgg
aagtaaaagtgtgcactggtaccagcagaagccaggccaggcccctgtggtggtcatccattatgatagtgaccggccctcag
ggatccctgagcgattctctggctccaactctgggaacacggccaccctgaccatcagcagggtcgaagccggggatgaggc
cgactattactgtcaggtgtgggatagtagtagtgatcatccttatgtcttcggaactgggaccaaggtcaccgtcctaggt
[SEQ ID NO: 297]
tctagaggtggtggtggtagcggcggcggcggctctggtggtggtggatccctcgagatggcc [SEQ ID NO: 305]
caggtgcagctggtgcagtctggagctgaggtgaagaagcctggggcctcagtgaaggtctcctgcaaggcttctggtt
acacctttaccaactatggtatcagctgggtgcgacaggcccctggacaagggcttgagtggatgggatggatcagcgc
ttacaatggtaacacaaactatgcacataagctccagggcagagtcaccatgaccacagacacatccacgagcacagc
caacatggagctgaggagcctgagacctgacgacactgccgtgtattactgtgcgcgctcttacttcggttctcatgatta
ctggggtcaaggtactctggtgaccgtctcctca [SEQ ID NO: 298]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
QPVLTQPPSVSVVPGKTARITCGGKNIGSKSVHWYQQKPGQAPVVVIHYDSDRP
SGIPERFSGSNSGNTATLTISRVEAGDEADYYCQVWDSSSDHPYVFGTGTKVTVL
G [SEQ ID NO: 299]
SRGGGGSGGGGSGGGGSLEMA [SEQ ID NO: 307]
QVQLVQSGAEVKKPGASVKVSCKASGYTFTNYGISWVRQAPGQGLEWMG
WISAYNGNTNYAHKLQGRVTMTTDTSTSTANMELRSLRPDDTAVYYCARSY
FGSHDYWGQGTLVTVSS [SEQ ID NO: 300]
TSGQAGQHHHHHHGAYPYDVPDYAS [SEQ ID NO: 308]

TABLE 76
ET200-124

DNA Sequence
(light chain variable region scFv linker heavy chain variable region His tag + HA tag)
tcctatgtgctgactcagccaccctcggtgtcagtggccccaggaaagacggccaggatttcctgtgggggaaacgacattgga
agtaaaagtgttttctggtatcagcagaggccaggccaggcccctgtgttggtcgtctatgatgatagcgaccggccctcagggc
tccctgagcgattctctggcttcaactctgggaacacggccaccctgaccatcagcagggtcgaagccggggatgaggccgac
tattactgtcaagtgtgggatagtagtagtgatcattatgtcttcggaactgggaccaaggtcaccgtcctaggt [SEQ ID
NO: 301]
tctagaggtggtggtggtagcggcggcggcggctctggtggtggtggatccctcgagatggcc [SEQ ID NO: 305]
caggtgcagctggtggagtctgggggaggcttggtacagcctggcaggtccctgagactctcctgtgcagcctctggatt
cacctttgatgattatgccatgcactgggtccggcaagctccagggaagggcctggagtgggtctcaggtattagttgga
atagtggtagcataggctatgcggactctgtgaagggccgattcaccatctccagagacaacgccaagaactccctgtat
ctgcaaatgaacagtctgagagctgaggacacggccttgtattactgtgcaaaagatataacctatggttcggggagtta
tggtgcttttgatatctggggccaagggacaatggtcaccgtctcttca [SEQ ID NO: 302]
ACTAGTGGCCAGGCCGGCCAGCACCATCACCATCACCATGGCGCATACCCGT
ACGACGTTCCGGACTACGCTTCT [SEQ ID NO: 306]
Amino Acid Sequence
(light chain variable region scFv linker heavy chain variable region His tag + HA tag)
SYVLTQPPSVSVAPGKTARISCGGNDIGSKSVFWYQQRPGQAPVLVVYDDSDRP
SGLPERFSGFNSGNTATLTISRVEAGDEADYYCQVWDSSSDHYVFGTGTKVTVL
G [SEQ ID NO: 303]
SRGGGGSGGGGSGGGGSLEMA [SEQ ID NO: 307]
QVQLVESGGGLVQPGRSLRLSCAASGFTFDDYAMHWVRQAPGKGLEWVSG
ISWNSGSIGYADSVKGRFTISRDNAKNSLYLQMNSLRAEDTALYYCAKDITY
GSGSYGAFDIWGQGTMVTVSS [SEQ ID NO: 304]
TSGQAGQHHHHHHGAYPYDVPDYAS [SEQ ID NO: 308]

TABLE 77
ET200-001

DNA Sequence
Cagtctgtgttgacgcagccaccctcagcgtctgggacccccgggcagagggtcaccatctcttgttctggaagcagctccaac
atcggaagtaatactgtaaactggtaccagcagctcccaggaacggcccccaaactcctcatctatagtaataatcagcggccct
caggggtccctgaccgattctctggctccaagtctggcacctcagcctccctggccatcagtgggctccagtctgaggatgagg
ctgattattactgtgcagcatgggatgacagcctgaatggttatgtcttcggaactgggaccaaggtcaccgtcctaggttctaga
ggtggtggtggtagcggcggcggcggctctggtggtggtggatccctcgagatggcccaggtgcagctacagcagtggggc
gcaggactgttgaagccttcggagaccctgtccctcacctgcgctgtgtatggtgggtccttcagtggttactactggagctggat
ccgccagcccccagggaaggggctggagtggattggggaaatcaatcatagtggaagcaccaactacaacccgtccctcaag
agtcgagtcaccatatcagtagacacgtccaagaaccagttctccctgaagctgagctctgtgaccgccgcggacacggccgt
gtattactgtgcgcgcgaaggtccgtacgacggtttcgattcttggggtcaaggtactctggtgaccgtctcctca [SEQ ID
NO: 593]
Amino Acid Sequence
QSVLTQPPSASGTPGQRVTISCSGSSSNIGSNTVNWYQQLPGTAPKLLIYSNNQRP
SGVPDRFSGSKSGTSASLAISGLQSEDEADYYCAAWDDSLNGYVFGTGTKVTVL
GSRGGGGSGGGGSGGGGSLEMAQVQLQQWGAGLLKPSETLSLTCAVYGGSFSG
YYWSWIRQPPGKGLEWIGEINHSGSTNYNPSLKSRVTISVDTSKNQFSLKLSSVT
AADTAVYYCAREGPYDGFDSWGQGTLVTVSS [SEQ ID NO: 594]

TABLE 78
ET200-002

DNA Sequence
Aattttatgctgactcagccccactctgtgtcggagtctccggggaagacggtaaccatctcctgcacccgcagcagtggcagc
attgccagcaactatgtgcagtggtaccagcagcgcccgggcagtgcccccaccactgtgatctatgaggataaccaaagacc
ctctggggtccctgatcggttctctggctccatcgacagctcctccaactctgcctccctcaccatctctggactgaagactgagg
acgaggctgactactactgtcagtcttatgatagcagcaattctgtggtattcggcggagggaccaagctgaccgtcctaggttct
agaggtggtggtggtagcggcggcggcggctctggtggtggtggatccctcgagatggcccaggtccagctggtacagtctg
gcactgaggtgaagaagcctggggcctcagtgagggtcgcctgcaaggcttctggttacccctttaacaaatatgacatcaactg
ggtgcgacaggcccctggacaagggcttgagtggatgggaggcatcatccctatctttcgtacaacaaactacgcacagaagtt
ccagggcagagtcacgattaccgcggacgaatccacgagcacagcctacatggagctgagcagcctgagatctgaggacac
ggccgtatattactgtgcgcgcgaatggttctactgggatatctggggtcaaggtactctggtgaccgtctcctca [SEQ ID
NO: 595]
Amino Acid Sequence
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSSNSVVFGGGTKLTVLG
SRGGGGSGGGGSGGGGSLEMAQVQLVQSGTEVKKPGASVRVACKASGYPFNK
YDINWVRQAPGQGLEWMGGIIPIFRTTNYAQKFQGRVTITADESTSTAYMELSSL
RSEDTAVYYCAREWFYWDIWGQGTLVTVSS [SEQ ID NO: 596]

TABLE 79
ET200-003

DNA Sequence
Cagtctgtgttgactcagccaccctcagtgtccgtgtccccaggacagacagccagcatctcctgctctggaaataaattgggga
ctaagtatgtttactggtatcagaagaggccaggccagtcccctgtgttggtcatgtatgaagataatcagcggccctcagggatc
ccggagcggttctctggctccaactctgggaacacagccactctgaccatcagagggacccagactgtggatgaggctgactat
tactgtcaggcgtgggactccgacactttcgtggtcttcggcggagggaccaaggtcaccgtcctaggttctagaggtggtggtg
gtagcggcggcggcggctctggtggtggtggatccctcgagatggccgaggtgcagctggtggagaccgggggaggcgtg
gtccagcctgggaggtccctgagactctcctgtgcagcctctggattcaccttcagtagttatggcatgcactgggtccgccagg
ctccaggcaaggggctggagtgggtggcagttatatcacatgatggaagtaataaatactacgcagactccgtgaagggccgat
tcaccatctccagagacaattccaaggacacgctgtatctgcaaatgaacagcctgagaggtgaggacacggccgtatattact
gtgcgcgctctaaccagtggtctggttacttctctttcgattactggggtcaaggtactctggtgaccgtctcctca [SEQ ID
NO: 597]
Amino Acid Sequence
QSVLTQPPSVSVSPGQTASISCSGNKLGTKYVYWYQKRPGQSPVLVMYEDNQRP
SGIPERFSGSNSGNTATLTIRGTQTVDEADYYCQAWDSDTFVVFGGGTKVTVLG
SRGGGGSGGGGSGGGGSLEMAEVQLVETGGGVVQPGRSLRLSCAASGFTFSSYG
MHWVRQAPGKGLEWVAVISHDGSNKYYADSVKGRFTISRDNSKDTLYLQMNS
LRGEDTAVYYCARSNQWSGYFSFDYWGQGTLVTVSS [SEQ ID NO: 598]

TABLE 80
ET200-006

DNA Sequence
Tcctatgtgctgactcagccaccctcagtgtcagtggccccaggaaagacggccaggattacctgtgggggaaacaacattgg
aagtaaaagtgtgcactggtaccagcagaagccaggccaggcccctgtggtggtcatccattatgatagcgaccggccctcag
ggatccctgagcgattctctggctccaactctgggaacacggccaccctgaccatcagcagggtcgaagccggggatgaggc
cgactattactgtcaggtgtgggatagtagtagtgatcatccttatgtcttcggaactgggaccaaggtcaccgtcctaggttctag
aggtggtggtggtagcggcggcggcggctctggtggtggtggatccctcgagatggccgaggtgcagctggtgcagtctgga
gctgaggtgaagaagcctggggcctcagtgaaggtctcctgcaaggcttctggttacacctttaccacctatggtatcagctgggt
gcgacaggcccctggacaagggcttgagtggatgggatggatcaacacttacaatggtcacacaaactatgcacagaagctcc
agggcagagccacaatgaccgcagacacatccacgaacacagcctacatggagctgaggagcctgagatctgacgacactg
ccgtgtattactgtgcgcgcgttatctacggttctggtgattactggggtcaaggtactctggtgaccgtctcctca [SEQ ID
NO: 599]
Amino Acid Sequence
SYVLTQPPSVSVAPGKTARITCGGNNIGSKSVHWYQQKPGQAPVVVIHYDSDRP
SGIPERFSGSNSGNTATLTISRVEAGDEADYYCQVWDSSSDHPYVFGTGTKVTVL
GSRGGGGSGGGGSGGGGSLEMAEVQLVQSGAEVKKPGASVKVSCKASGYTFTT
YGISWVRQAPGQGLEWMGWINTYNGHTNYAQKLQGRATMTADTSTNTAYMEL
RSLRSDDTAVYYCARVIYGSGDYWGQGTLVTVSS [SEQ ID NO: 600]

TABLE 81
ET200-007

DNA Sequence
Tcctatgtgctgactcagccactctcagtgtcagtggccccaggaaagacggccaggattacctgtgggggaaacaacattgg
aagtaaaactgtgcactggtaccagcagaagccaggccaggcccctgtgctggtcatctattatgatagcgaccggccctcagg
gatccctgagcgattctctggctccaactctgggaacacggccaccctgaccatcagcagggtcgaagccggggatgaggcc
gactattactgtcaggtgtgggatagtagtagtgatcatcgggtgttcggcggagggaccaagctgaccgtcctaggttctagag
gtggtggtggtagcggcggcggcggctctggtggtggtggatccctcgagatggcccaggtgcagctgcaggagtcgggcc
caggactggtgaagccttcggagaccctgtccctcacctgcaatgtctctggttactccatcagcagtggttacttttggggctgga
tccggcagcccccagggaaggggctggagtggattgggagtatctatcatagtaggagcacctactacaacccgtccctcaag
agtcgagtcaccatatcagtagacacgtccaagaaccagttctccctgaagctgaactctgtgaccgccgcagacacggccgtg
tattactgtgcgcgcggttacggttacttcgattactggggtcaaggtactctggtgaccgtctcctca [SEQ ID NO: 601]
Amino Acid Sequence
SYVLTQPLSVSVAPGKTARITCGGNNIGSKTVHWYQQKPGQAPVLVIYYDSDRP
SGIPERFSGSNSGNTATLTISRVEAGDEADYYCQVWDSSSDHRVFGGGTKLTVLG
SRGGGGSGGGGSGGGGSLEMAQVQLQESGPGLVKPSETLSLTCNVSGYSISSGYF
WGWIRQPPGKGLEWIGSIYHSRSTYYNPSLKSRVTISVDTSKNQFSLKLNSVTAA
DTAVYYCARGYGYFDYWGQGTLVTVSS [SEQ ID NO: 602]

TABLE 82
ET200-008

DNA Sequence
Caatctgccctgactcagcctgcctccgtgtctgggtctcctggacagtcgatcaccatctcctgcactggaaccagcagtgacg
ttggtggttataactatgtctcctggtaccaacaacacccaggcaaagcccccaaactcatgatttatgatgtcagtaatcggccct
caggggtttctaatcgcttctctggctccaagtctggcaacacggcctccctgaccatctctgggctccaggctgaggacgaggc
tgattattactgcagctcatatacaagcagcagcacttcgaaggtgttcggcggagggaccaagctgaccgtcctaggttctaga
ggtggtggtggtagcggcggcggcggctctggtggtggtggatccctcgagatggccgaggtgcagctggtggagtctgggg
gaggtgtggtacggcctggggggtccctgagactctcctgtgcagcctctggattcacctttggtgattatggcatgagctgggtc
cgccaagctccagggaaggggctggagtgggtctctggtattaattggaatggtggtagcacaggttatgcagactctgtgaag
ggccgattcaccatctccagagacaacgccaagaactccctgtatctgcaaatgaacagtctgagagccgaggacacggccgt
atattactgtgcgcgctctaaatacaacttccatgtttactacgattactggggtcaaggtactctggtgaccgtctcctca [SEQ
ID NO: 603]
Amino Acid Sequence
QSALTQPASVSGSPGQSITISCTGTSSDVGGYNYVSWYQQHPGKAPKLMIYDVSN
RPSGVSNRFSGSKSGNTASLTISGLQAEDEADYYCSSYTSSSTSKVFGGGTKLTVL
GSRGGGGSGGGGSGGGGSLEMAEVQLVESGGGVVRPGGSLRLSCAASGFTFGD
YGMSWVRQAPGKGLEWVSGINWNGGSTGYADSVKGRFTISRDNAKNSLYLQM
NSLRAEDTAVYYCARSKYNFHVYYDYWGQGTLVTVSS [SEQ ID NO: 604]

TABLE 83
ET200-009

DNA Sequence
Cagtctgtgttgacgcagccaccctcagcgtctgggacccccgggcagacagtcaccatctcttgttctggaagcaactccaac
atcggaagtaattatgtatactggtaccagcagctcccaggaacggcccccaaactcctcatctataggaataatcagcggccct
caggggtccctgaccgattctcaggctccaagtctggcacctcagcctccctggccatcagtgggctccgctccgaggatgag
gctgattattactgtgcagcatgggatgacagcctgagtgcttatgtcttcggaactgggaccaaggtcaccgtcctaggttctag
aggtggtggtggtagcggcggcggcggctctggtggtggtggatccctcgagatggcccaggtgcagctggtgcagtctgga
gctgaggtgaagaagcctggggcctcagtgaaggtctcctgcaaggcttctggttacacctttaccagctatggtatcagctggg
tgcgacaggcccctggacaagggcttgagtggatgggatggatcagcgcttacaatggtaacacaaactatgcacagaagctc
cagggcagagtcaccatgaccacagacacatccacgagcacagcctacatggagctgaggagcctgagatctgacgacactg
ccgtgtattactgtgcgcgctcttctggtaacatggtttcttggaaagatatgtggggtcaaggtactctggtgaccgtctcctca
[SEQ ID NO: 605]
Amino Acid Sequence
QSVLTQPPSASGTPGQTVTISCSGSNSNIGSNYVYWYQQLPGTAPKLLIYRNNQR
PSGVPDRFSGSKSGTSASLAISGLRSEDEADYYCAAWDDSLSAYVFGTGTKVTVL
GSRGGGGSGGGGSGGGGSLEMAQVQLVQSGAEVKKPGASVKVSCKASGYTFTS
YGISWVRQAPGQGLEWMGWISAYNGNTNYAQKLQGRVTMTTDTSTSTAYMEL
RSLRSDDTAVYYCARSSGNMVSWKDMWGQGTLVTVSS [SEQ ID NO: 606]

TABLE 84
ET200-010

DNA Sequence
Caatctgccctgactcagcctgcctccgtgtctgggtctcctggacagtcgatcaccatctcctgcactggaaccagcagtgacg
ttggtggttataactctgtctcctggtaccaacaacacccaggcaaagcccccagactcatgatttatgatgtcagtaatcggccct
caggggtttctaatcgcttctctggctccaagtctggcaacacggcctccctgaccatctctgggctccaggctgaggacgaggc
tgattattactgcagctcatatacaagcagcagcacccctttagtcttcggaactgggaccaaggtcaccgtcctaggttctagag
gtggtggtggtagcggcggcggcggctctggtggtggtggatccctcgagatggcccaggtgcagctggtgcagtctggggc
tgaggtgaagaagcctggggcctcagtgaaggtctcctgcaaggcttctggttacacctttaccagctatggtatcagctgggtg
cgacaggcccctggacaagggcttgagtggatgggatggatcagcgcttacaatggtaacacaaactatgcacagaagctcca
gggcagagtcaccatgaccacagacacatccacgagcacagcctacatggagctgaggagcctgagatctgacgacacggc
cgtgtattactgtgcgcgcggtgctgttgcttaccatgattggggtcaaggtactctggtgaccgtctcctca [SEQ ID NO:
607]
Amino Acid Sequence
QSALTQPASVSGSPGQSITISCTGTSSDVGGYNSVSWYQQHPGKAPRLMIYDVSN
RPSGVSNRFSGSKSGNTASLTISGLQAEDEADYYCSSYTSSSTPLVFGTGTKVTVL
GSRGGGGSGGGGSGGGGSLEMAQVQLVQSGAEVKKPGASVKVSCKASGYTFTS
YGISWVRQAPGQGLEWMGWISAYNGNTNYAQKLQGRVTMTTDTSTSTAYMEL
RSLRSDDTAVYYCARGAVAYHDWGQGTLVTVSS [SEQ ID NO: 608]

TABLE 85
ET200-011

DNA Sequence
Cagtctgtcgtgacgcagccgccctcagtgtctgcggccccaggacagagggtcaccatctcctgctctggaagcagctccaa
catttcgatttatgatgtatcctggtatcagcagctcccaggaacagcccccaaactcctcatttatggcaataataagcgaccctc
ggggattgctgaccgattctctggctccacgtctggcacgtcagccaccctgggcatcaccggactccagactggggacgagg
ccgattattactgcggaacatgggatgacagtctgagtgggggggtgttcggcggagggaccaagctgaccgtcctaggttcta
gaggtggtggtggtagcggcggcggcggctctggtggtggtggatccctcgagatggcccagatgcagctggtgcaatctgg
ggctgaggtgaagaagcctgggtcctcggtgaaggtctcctgcgaggcttctggaggcaccctcagcagctatgctatcaactg
ggtgcgacaggcccctggacaagggcttgagtggatgggagggatcatccctatgtttggtacagcacactacgcacagaagt
tccagggcagagtcacgattaccgcggacgaatccacgaaaacagcctacatggagctgagcagcctgagatctgaggacac
tgccgtgtattactgtgcgcgcggtgttcattacgcttctttcgatcattggggtcaaggtactctggtgaccgtctcctca [SEQ
ID NO: 609]
Amino Acid Sequence
QSVVTQPPSVSAAPGQRVTISCSGSSSNISIYDVSWYQQLPGTAPKLLIYGNNKRP
SGIADRFSGSTSGTSATLGITGLQTGDEADYYCGTWDDSLSGGVFGGGTKLTVLG
SRGGGGSGGGGSGGGGSLEMAQMQLVQSGAEVKKPGSSVKVSCEASGGTLSSY
AINWVRQAPGQGLEWMGGIIPMFGTAHYAQKFQGRVTITADESTKTAYMELSSL
RSEDTAVYYCARGVHYASFDHWGQGTLVTVSS [SEQ ID NO: 610]

TABLE 86
ET200-012

DNA Sequence
Cagtctgtgttgacgcagccgccctcagtgtctgcggccgcaggacagaaggtcaccatctcctgctctggaagcgactccaa
cattgggaataattatgtgtcctggtatcaacacctcccagggacagcccccaaactcctcatttatgacgttaaaaatcgaccctc
agggattcctgaccggttctccggctccaagtctggctcgtcagccaccctaggcatcgccggactccagcctggggacgagg
ccgattattactgcggaacatgggacagtcggctggatgcctatgtcttcggaactgggaccaaggtcaccgtcctaggttctag
aggtggtggtggtagcggcggcggcggctctggtggtggtggatccctcgagatggcccagatgcagctggtgcaatctgga
gctgaggtgaagaagcctggggcctcagtgaaggtctcctgcaagacttctggtttcccctttaatatctttggaatcacctgggtg
cgacaggcccctggacaagggcttgagtggatgggatggatcagcggttacaacggtaacacagactacccacagaagttcc
agggcagagtcaccatgtccacagacacatccacgagtacagcctacatggagctgaggaacctgaaatctgacgacacggc
cgtgtattactgtgcgcgcggtgcttacggtggtatggatacttggggtcaaggtactctggtgaccgtctcctca [SEQ ID
NO: 611]
Amino Acid Sequence
QSVLTQPPSVSAAAGQKVTISCSGSDSNIGNNYVSWYQHLPGTAPKLLIYDVKNR
PSGIPDRFSGSKSGSSATLGIAGLQPGDEADYYCGTWDSRLDAYVFGTGTKVTVL
GSRGGGGSGGGGSGGGGSLEMAQMQLVQSGAEVKKPGASVKVSCKTSGFPFNI
FGITWVRQAPGQGLEWMGWISGYNGNTDYPQKFQGRVTMSTDTSTSTAYMELR
NLKSDDTAVYYCARGAYGGMDTWGQGTLVTVSS [SEQ ID NO: 612]

TABLE 87
ET200-013

DNA Sequence
Cagtctgtcgtgacgcagccgccctcagtgtctggggccccagggcagagggtcaccatctcctgcactgggagcacctcca
acatcggggcaggttatgatgtacactggtatcagcagcttccaggaacagcccccaaactcctcatctatactaacaactttcgg
ccctcaggggtccctgaccgattctctgcctccaagtctggcacttcagcttccctggccatcactggtctccaggctgaggatga
ggctgattattactgcggaacatgggatagcagcctgagtgccgttgtgttcggcggagggaccaagctgaccgtcctaggttct
agaggtggtggtggtagcggcggcggcggctctggtggtggtggatccctcgagatggccgaggtgcagctggtggagtctg
gaactgaggtgaagaagcctggggcctcagtgaaagtctcctgcaaggcttctggttacatgtttaccagttatggtctcaactgg
gtgcgacaggcccctggacaagggcttgagtggatgggatggatcagcgctaacaatggtaagacaaattatgctaagaaattc
caggacagagtcaccatgaccagagacacttccacgagcacaggctacatggaactgaggagcctgagatctgacgacacg
gccgtatattactgtgcgcgccatatcggtggttcttacttcgatcgttggggtcaaggtactctggtgaccgtctcctca [SEQ
ID NO: 613]
Amino Acid Sequence
QSVVTQPPSVSGAPGQRVTISCTGSTSNIGAGYDVHWYQQLPGTAPKLLIYTNNF
RPSGVPDRFSASKSGTSASLAITGLQAEDEADYYCGTWDSSLSAVVFGGGTKLT
VLGSRGGGGSGGGGSGGGGSLEMAEVQLVESGTEVKKPGASVKVSCKASGYMF
TSYGLNWVRQAPGQGLEWMGWISANNGKTNYAKKFQDRVTMTRDTSTSTGY
MELRSLRSDDTAVYYCARHIGGSYFDRWGQGTLVTVSS [SEQ ID NO: 614]

TABLE 88
ET200-014

DNA Sequence
Tcctatgtgctgactcagccaccctcagtgtcagtggccccaggaaagacggccaggattacctgtgggggaaacaacattgg
aagtaaaagtgtgcactggtaccagcagaagccaggccaggcccctgtgctggtcatctattatgatagcgaccggccctcagg
gatccctgagcgattctctggctccaactctgggaacacggccaccctgaccatcagcagggtcgaagccggggatgaggcc
gactattactgtcaggtgtgggatagtagtagtgatcattatgtcttcggaactgggaccaaggtcaccgtcctaggttctagaggt
ggtggtggtagcggcggcggcggctctggtggtggtggatccctcgagatggccgaggtgcagctggtggagactggggga
ggcttggtacagcctggggggtccctgagactctcctgtgcagcctctggattcacctttagcagctatgccatgagctgggtcc
gccaggctccagggaaggggctggagtgggtctcagctattagtggtagtgatggtagcacatactacgcagactccgtgaag
ggccggttcaccatctccagagacaattccaagaacacgctgtatctgcaaatgaacagcctgagagacgaggacacggccgt
atattactgtgcgcgctctcatgaagctaacctggttggtgattggtggggtcaaggtactctggtgaccgtctcctca [SEQ
ID NO: 615]
Amino Acid Sequence
SYVLTQPPSVSVAPGKTARITCGGNNIGSKSVHWYQQKPGQAPVLVIYYDSDRPS
GIPERFSGSNSGNTATLTISRVEAGDEADYYCQVWDSSSDHYVFGTGTKVTVLGS
RGGGGSGGGGSGGGGSLEMAEVQLVETGGGLVQPGGSLRLSCAASGFTFSSYA
MSWVRQAPGKGLEWVSAISGSDGSTYYADSVKGRFTISRDNSKNTLYLQMNSL
RDEDTAVYYCARSHEANLVGDWWGQGTLVTVSS [SEQ ID NO: 616]

TABLE 89
ET200-015

DNA Sequence
Cagtctgtggtgactcagccaccctcagtgtcagtggccccaggaaagacggccaggattacctgtgggggaaacaacattg
gaagtaaaagtgtgcactggtaccagcagaagccaggccaggcccctgtgctggtcatctattatgatagcgaccggccctcag
ggatccctgagcgattctctggctccaactctgggaacacggccaccctgaccatcagcagggtcgaagccggggatgaggc
cgactattactgtcaggtgtgggatagtagtagtgatgtggtattcggcggagggaccaagctgaccgtcctaggttctagaggt
ggtggtggtagcggcggcggcggctctggtggtggtggatccctcgagatggccgaggtccagctggtacagtctggagctg
aggtgaagaagcctggggcctcagtgaaggtctcctgcaaggcttctggttacacctttaccagctacggtatcagctgggtgcg
acaggcccctggacaagggcttgagtggatgggatggatcagcgcttacaatggtaacacaaactatgcacagaagctccagg
gcagagtcaccatgaccacagacacatccacgagcacagcctacatggagctgaggagcctgagatctgacgacacggccgt
gtattactgtgcgcgctggggtggtttcggtgctgttgatcattggggtcaaggtactctggtgaccgtctcctca [SEQ ID
NO: 617]
Amino Acid Sequence
QSVVTQPPSVSVAPGKTARITCGGNNIGSKSVHWYQQKPGQAPVLVIYYDSDRP
SGIPERFSGSNSGNTATLTISRVEAGDEADYYCQVWDSSSDVVFGGGTKLTVLGS
RGGGGSGGGGSGGGGSLEMAEVQLVQSGAEVKKPGASVKVSCKASGYTFTSYG
ISWVRQAPGQGLEWMGWISAYNGNTNYAQKLQGRVTMTTDTSTSTAYMELRS
LRSDDTAVYYCARWGGFGAVDHWGQGTLVTVSS [SEQ ID NO: 618]

TABLE 90
ET200-016

DNA Sequence
Tcttctgagctgactcaggaccctgctgtgtctgtggccttgggacagacagtcaagatcacgtgccaaggagacagcctcaca
gactaccatgcaacctggtaccagcagaagccaggacaggcccctgtcgctgtcatctatgctacaaacaaccggcccactgg
gatcccagaccgattctctggttccagttccggaaacacagcttctttgaccatcactggggctcaggcggaagatgaggctgac
tattactgtaattcccgggacagcggcacggacgaagtgttattcggcggagggaccaagctgaccgtcctaggttctagaggt
ggtggtggtagcggcggcggcggctctggtggtggtggatccctcgagatggccgaggtgcagctggtggagactggggga
ggcctggtcaagcctggggggtccctgagactctcctgtgcagcctctggattcaccttcagtagctatagcatgaactgggtcc
gccaggctccagggaaggggctggagtgggtctcatccattagtagtagtagtagttacatatactacgcagactcagtgaagg
gccgattcaccatctccagagacaacgccaagaactcactgtatctgcaaatgaacagcctgagagccgaggacacggccgtg
tattactgtgcgcgcggtcagggttacgattactggggtcaaggtactctggtgaccgtctcctca [SEQ ID NO: 619]
Amino Acid Sequence
SSELTQDPAVSVALGQTVKITCQGDSLTDYHATWYQQKPGQAPVAVIYATNNRP
TGIPDRFSGSSSGNTASLTITGAQAEDEADYYCNSRDSGTDEVLFGGGTKLTVLG
SRGGGGSGGGGSGGGGSLEMAEVQLVETGGGLVKPGGSLRLSCAASGFTFSSYS
MNWVRQAPGKGLEWVSSISSSSSYIYYADSVKGRFTISRDNAKNSLYLQMNSLR
AEDTAVYYCARGQGYDYWGQGTLVTVSS [SEQ ID NO: 620]

TABLE 91
ET200-017

DNA Sequence
Tcctatgtgctgactcagccaccctcggtgtcagtggccccaggaaagacggccaggattacctgtgggggaaacaacattgg
aagtaaaagtgtgcactggtaccagcagaagccaggccaggcccctgtgctggtcgtctatgatgatagcgaccggccctcag
ggatccctgagcgattctctggctccaactctgggaacacggccaccctgagcatcagcagggtcgaagccggggatgaggc
cgactattactgtcaggtgtgggatagtagtagtgatcatactgtcttcggaactgggaccaaggtcaccgtcctaggttctagag
gtggtggtggtagcggcggcggcggctctggtggtggtggatccctcgagatggcccaggtgcagctacagcagtggggcg
caggactgttgaagccttcggagaccctgtccctcacctgcgctgtctatggtgggtccttcagtggttactactggagctggatc
cgccagcccccagggaaggggctggagtggattggggaaatcaatcatagtggaagcaccaactacaacccgtccctcaaga
gtcgagtcaccatatcagtagacacgtccaagaaccagttctccctgaagctgagctctgtgaccgccgcggacacggccgtgt
attactgtgcgcgctactacccgggtatggatatgtggggtcaaggtactctggtgaccgtctcctca [SEQ ID NO: 621]
Amino Acid Sequence
SYVLTQPPSVSVAPGKTARITCGGNNIGSKSVHWYQQKPGQAPVLVVYDDSDRP
SGIPERFSGSNSGNTATLSISRVEAGDEADYYCQVWDSSSDHTVFGTGTKVTVLG
SRGGGGSGGGGSGGGGSLEMAQVQLQQWGAGLLKPSETLSLTCAVYGGSFSGY
YWSWIRQPPGKGLEWIGEINHSGSTNYNPSLKSRVTISVDTSKNQFSLKLSSVTA
ADTAVYYCARYYPGMDMWGQGTLVTVSS [SEQ ID NO: 622]

TABLE 92
ET200-018

DNA Sequence
Caggctgtgctgactcagccgccctcaacgtctgggacccccgggcagagggtcaccatctcttgttctggaagcagctccaa
catcgggagaaatggtgtaaactggtaccagcagctcccaggagcggcccccaaagtcctcatctataatgataatcagcgacc
ctcaggggtccctgaccgagtctctggctcccagtctggctcctcaggcaccctggccatcgatgggcttcggtctgaggatga
ggctgattattactgtgcggcatgggatgacagcctgcatggtgtggtattcggcggagggaccaagctgaccgtcctaggttct
agaggtggtggtggtagcggcggcggcggctctggtggtggtggatccctcgagatggcccaggtccagctggtacagtctg
gggctgaggtgaagaagcctggggcctcagtgaaggtctcctgcaaggtttccggatacaccctcaatgaattatccatgcactg
ggtgcgacaggctcctggaaaagggcttgagtggatgggaggttttgatcctgaagatggtgaaacaatctacgcacagaagtt
ccagggcagagtcaccatgaccgaggacacatctacagacacagcctacatggagctgagcagcctgagatctgaggacact
gccgtgtattactgtgcgcgcggtggttacggtgattcttggggtcaaggtactctggtgaccgtctcctca [SEQ ID NO:
623]
Amino Acid Sequence
QAVLTQPPSTSGTPGQRVTISCSGSSSNIGRNGVNWYQQLPGAAPKVLIYNDNQR
PSGVPDRVSGSQSGSSGTLAIDGLRSEDEADYYCAAWDDSLHGVVFGGGTKLTV
LGSRGGGGSGGGGSGGGGSLEMAQVQLVQSGAEVKKPGASVKVSCKVSGYTL
NELSMHWVRQAPGKGLEWMGGFDPEDGETIYAQKFQGRVTMTEDTSTDTAYM
ELSSLRSEDTAVYYCARGGYGDSWGQGTLVTVSS [SEQ ID NO: 624]

TABLE 93
ET200-019

DNA Sequence
Aattttatgctgactcagccccactctgtgtcggagtctccggggaagacggtaaccatctcctgcacccgcagcagtggcagc
attgccagcaactatgtgcagtggtaccagcagcgcccgggcagtgcccccaccactgtgatctatgaggataaccaaagacc
ctctggggtccctgatcggttctctggctccatcgacagctcctccaactctgcctccctcaccatctctggactgaagactgagg
acgaggctgactactactgtcagtcttatgatagcagcaattcttgggtgttcggcggagggaccaagctgaccgtcctaggttct
agaggtggtggtggtagcggcggcggcggctctggtggtggtggatccctcgagatggcccaggtgcagctggtgcaatctg
gggctgaggtgaagaggcctgggtcctcggtgaaggtctcctgcacggcttctggaggcaccttcagcagcgatgctatcagct
gggtgcgacaggcccctggacaagggcttgagtggatgggaggaatcatccctatgtttggtacagcaaactacgcacagaag
ttccagggcagagtcacgattaccgcggacgaatccacgagcacagcctacatggagctgagcagcctgagatctgaggaca
cggccgtgtattactgtgcgcgcgaaggttactactacccgtctgcttacctgggttctgttctgaacgacatctcttctgtttacgat
gaatggggtcaaggtactctggtgaccgtctcctca [SEQ ID NO: 625]
Amino Acid Sequence
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSSNSWVFGGGTKLTVL
GSRGGGGSGGGGSGGGGSLEMAQVQLVQSGAEVKRPGSSVKVSCTASGGTFSS
DAISWVRQAPGQGLEWMGGIIPMFGTANYAQKFQGRVTITADESTSTAYMELSS
LRSEDTAVYYCAREGYYYPSAYLGSVLNDISSVYDEWGQGTLVTVSS [SEQ ID
NO: 626]

TABLE 94
ET200-020

DNA Sequence
Cagtctgtcgtgacgcagccgccctcagtgtctgcggccccaggacagaaggtcaccatctcctgctctggaagcacctccaa
cattggaaataatgatgtatcctggtaccagcagctcccaggaacagcccccaaactcctcatttatgacaataataagcgaccct
cagggattcctgaccgattctctggctccaagtctggcacgtcagccaccctgggcatcaccggactccagactggggacgag
gccgattattactgcggaacatgggatagcagcgtgagtgcttcttgggtcttcggcagagggaccaagctgaccgtcctaggtt
ctagaggtggtggtggtagcggcggcggcggctctggtggtggtggatccctcgagatggcccaggtgcagctggtgcagtct
ggagctgaggtgaagaagcctggggcctcagtgaaggtctcctgcaaggcttctggttacacctttaccagctatggtatcagct
gggtgcgacaggcccctggacaagggcttgagtggatgggatggatcagcgcttacaatggtaacacaaactatccacagaa
gctccagggcagagtcaccatgaccacagacccatccacgagcacagcctacatggagctgaggagcctgagatctgacgac
acggccgtgtattactgtgcgcgctctatgacttctttcgattactggggtcaaggtactctggtgaccgtctcctca [SEQ ID
NO: 627]
Amino Acid Sequence
QSVVTQPPSVSAAPGQKVTISCSGSTSNIGNNDVSWYQQLPGTAPKLLIYDNNKR
PSGIPDRFSGSKSGTSATLGITGLQTGDEADYYCGTWDSSVSASWVFGRGTKLTV
LGSRGGGGSGGGGSGGGGSLEMAQVQLVQSGAEVKKPGASVKVSCKASGYTFT
SYGISWVRQAPGQGLEWMGWISAYNGNTNYPQKLQGRVTMTTDPSTSTAYMEL
RSLRSDDTAVYYCARSMTSFDYWGQGTLVTVSS [SEQ ID NO: 628]

TABLE 95
ET200-021

DNA Sequence
Cagtctgtgttgacgcagccgccctcagtgtctgcggccccaggacagaaggtcaccatctcctgctctggaagcaactccaa
cattgggaataattatgtatcctggtatcagcaactcccagggacagcccccaaactcctcatttatgacaataataagcgaccctc
agggattcctgaccgattctctggctccaggtctggcacgtcagccaccctgggcatcaccggactccagactggggacgagg
ccgattattactgcggaacatggaataccactgtgactcctggctatgtcttcggaactgggaccaaggtcaccgtcctaggttcta
gaggtggtggtggtagcggcggcggcggctctggtggtggtggatccctcgagatggccgaagtgcagctggtgcagtctgg
agctgaggtgaagaagcctggggcctcagtgaaggtctcctgcaaggcttctggttacacctttaccagctatggtatcagctgg
gtgcgacaggcccctggacaagggcttgagtggatgggatggatcagcgcttacaatggtaacacaaactatgcacagaagct
ccagggcagagtcaccatgaccacagacacatccacgagcacagcctacatggagctgaggagcctgagatctgacgacac
cgccatgtattactgtgcgcgctctgtttacgacctggatacttggggtcaaggtactctggtgaccgtctcctca [SEQ ID
NO: 629]
Amino Acid Sequence
QSVLTQPPSVSAAPGQKVTISCSGSNSNIGNNYVSWYQQLPGTAPKLLIYDNNKR
PSGIPDRFSGSRSGTSATLGITGLQTGDEADYYCGTWNTTVTPGYVFGTGTKVTV
LGSRGGGGSGGGGSGGGGSLEMAEVQLVQSGAEVKKPGASVKVSCKASGYTFT
SYGISWVRQAPGQGLEWMGWISAYNGNTNYAQKLQGRVTMTTDTSTSTAYME
LRSLRSDDTAMYYCARSVYDLDTWGQGTLVTVSS [SEQ ID NO: 630]

TABLE 96
ET200-022

DNA Sequence
cagtctgtcgtgacgcagccgccctcagtgtctgcggccccaggacagaaggtcaccatctcctgctctggaagcagctccaa
cattgggaataattatgtatcctggtaccagcagctcccaggaacagcccccaaactcctcatttatgacaataataagcgaccct
cagggattcctgaccgattctctggctccaagtctggcacgtcagccaccctgggcatcaccggactccagactggggacgag
gccgattattactgcggaacatgggatagcagcctgggggccccttatgtcttcggaactgggaccaaggtcaccgtcctaggtt
ctagaggtggtggtggtagcggcggcggcggctctggtggtggtggatccctcgagatggccgaggtgcagctggtgcagtc
ttggggaggctcggaacagcctggcaggtccctgagactctcctgtgcagcctctggattcacctttgatgattatgccatgcact
gggtccggcaagctccagggaagggcctggagtgggtctcaggtattagttggaatagcggtagcataggctatgcggactct
gtgaagggccgattcaccatctccagagacaacgccaagaattccctgtatctgcaaatgaacagtctgagagctgaggacacc
gccatgtattactgtgcgcgctaccgtcaggttggttctgcttacgattcttggggtcaaggtactctggtgaccgtctcctca
[SEQ ID NO: 631]
Amino Acid Sequence
QSVVTQPPSVSAAPGQKVTISCSGSSSNIGNNYVSWYQQLPGTAPKLLIYDNNKR
PSGIPDRFSGSKSGTSATLGITGLQTGDEADYYCGTWDSSLGAPYVFGTGTKVTV
LGSRGGGGSGGGGSGGGGSLEMAEVQLVQSWGGSEQPGRSLRLSCAASGFTFD
DYAMHWVRQAPGKGLEWVSGISWNSGSIGYADSVKGRFTISRDNAKNSLYLQM
NSLRAEDTAMYYCARYRQVGSAYDSWGQGTLVTVSS [SEQ ID NO: 632]

TABLE 97
ET200-023

DNA Sequence
ctgcctgtgctgactcagccaccctcggtgtcagtggccccaggaaagacggccaggattacctgtgggggaaacaacattgg
aagtaaaagtgtgcactggtatcagcagaagccaggccaggcccctgtgctggtcgtctatgctgatagcgaccggccctcag
ggatccctgagcgattctctggctccaactctgggaacacggccaccctgaccatcagcagggtcgaagccggggatgaggc
cgactattactgtcaggtgtgggatagtagtagttatcataattatgtcttcggaactgggaccaaggtcaccgtcctaggttctaga
ggtggtggtggtagcggcggcggcggctctggtggtggtggatccctcgagatggccgaggtgcagctggtgcagtctggag
ctgaggtgaagaagcctggggcctcagtgaaggtctcctgcaaggcttctggttacacctttaccagctatggtatcagctgggt
gcgacaggcccctggacaagggcttgagtggatgggatggatcagcgcttacaatggtaacacaaactatgcacagaagctcc
agggcagagtcaccatgaccacagacacatccacgagcacagcctacatggagctgagcagcctgagatctgaggacaccg
ccatgtattactgtgcgcgctactggggtttcggtgtttctgatcgttggggtcaaggtactctggtgaccgtctcctca [SEQ
ID NO: 633]
Amino Acid Sequence
LPVLTQPPSVSVAPGKTARITCGGNNIGSKSVHWYQQKPGQAPVLVVYADSDRP
SGIPERFSGSNSGNTATLTISRVEAGDEADYYCQVWDSSSYHNYVFGTGTKVTVL
GSRGGGGSGGGGSGGGGSLEMAEVQLVQSGAEVKKPGASVKVSCKASGYTFTS
YGISWVRQAPGQGLEWMGWISAYNGNTNYAQKLQGRVTMTTDTSTSTAYMEL
SSLRSEDTAMYYCARYWGFGVSDRWGQGTLVTVSS [SEQ ID NO: 634]

TABLE 98
ET200-024

DNA Sequence
aattttatgctgactcagccccactctgtgtcggagtctccggggaagacggtaaccatctcctgcaccggcagcagtggcagc
attgccagcaactatgtgcagtggtaccagcagcgcccgggcagtgcccccaccactgtgatctatgaggataaccaaagacc
ctctggggtccccgatcggttctctggctccatcgacagctcctccaactctgcctccctcaccatctctggactgaagactgagg
acgaggctgactactactgtcagtcttatgacagcagcaatctttgggtgttcggcggagggaccaagctgaccgtcctaggttct
agaggtggtggtggtagcggcggcggcggctctggtggtggtggatccctcgagatggcccagatgcagctggtgcagtctg
gggctgaggtgaagaagcctgggtcctcggtgaaggtctcctgcaaggcttctggaggcaccttcagcagctatgctatcagct
gggtgcgacaggcccctggacaagggcttgagtggatgggagggatcatccctatctttggtacagcaaactacgcacagaag
ttccagggcagagtcacgattaccgcggacgaatccacgagcacagcctacatggagctgagcagcctgagatctgaggaca
ctgccgtgtattactgtgcgcgctacaactactactactacgattcttggggtcaaggtactctggtgaccgtctcctca [SEQ
ID NO: 635]
Amino Acid Sequence
NFMLTQPHSVSESPGKTVTISCTGSSGSIASNYVQWYQQRPGSAPTTVIYEDNQR
PSGVPDRFSGSIDSSSNSASLTISGLKTEDEADYYCQSYDSSNLWVFGGGTKLTVL
GSRGGGGSGGGGSGGGGSLEMAQMQLVQSGAEVKKPGSSVKVSCKASGGTFSS
YAISWVRQAPGQGLEWMGGIIPIFGTANYAQKFQGRVTITADESTSTAYMELSSL
RSEDTAVYYCARYNYYYYDSWGQGTLVTVSS [SEQ ID NO: 636]

TABLE 99
ET200-025

DNA Sequence
gacatccagatgacccagtctccatcctccctgtctgcatctgtaggagacagagtcaccatcacttgccgggcaagtcagagca
ttagcagctatttaaattggtatcagcagaaaccagggaaagcccctaagctcctgatctatgctgcatccagtttgcaaagtggg
gtcccatcaaggttcagtggcagtggatctgggacagatttcactctcaccatcagcagtctgcaacctgaagattttgcaacttac
tactgtcaacagagttacagtaccccattcactttcggccctgggaccaaagtggatatcaaacgttctagaggtggtggtggtag
cggcggcggcggctctggtggtggtggatccctcgagatggccgaggtgcagctggtgcagtctggggctgaggtgaagaa
gcctgggtcctcggtgaaggtctcctgcaaggcttctggaggcaccttcagcagctatgctatcagctgggtgcgacaggcccc
tggacaagggcttgagtggatgggagggatcatccctatctttggtacagcaaactacgcacagaagttccagggcagagtcac
gattaccgcggacgaatccacgagcacagcctacatggagctgagcagcctgagatctgaggacaccgccatgtattactgtg
cgcgctactggggttacgactcttacgatgaatggggtcaaggtactctggtgaccgtctcctca [SEQ ID NO: 637]
Amino Acid Sequence
DIQMTQSPSSLSASVGDRVTITCRASQSISSYLNWYQQKPGKAPKLLIYAASSLQS
GVPSRFSGSGSGTDFTLTISSLQPEDFATYYCQQSYSTPFTFGPGTKVDIKRSRGG
GGSGGGGSGGGGSLEMAEVQLVQSGAEVKKPGSSVKVSCKASGGTFSSYAISW
VRQAPGQGLEWMGGIIPIFGTANYAQKFQGRVTITADESTSTAYMELSSLRSEDT
AMYYCARYWGYDSYDEWGQGTLVTVSS [SEQ ID NO: 638]

TABLE 100
ET200-026

DNA Sequence
aattttatgctgactcagccccactctgtgtcggagtctccggggaagacggtaaccatctcctgcaccggcagcagtggcagc
attgccagcaactatgtgcagtggtaccagcagcgcccgggcagtgcccccaccactgtgatctatgaggataaccaaagacc
ctctggggtccctgatcggttctctggctccatcgacagctcctccaactctgcctccctcaccatctctggactgaagactgagg
acgaggctgactactactgtcagtcttatgatagcagcaattgggtgttcggcggagggaccaagctgaccgtcctaggttctag
aggtggtggtggtagcggcggcggcggctctggtggtggtggatccctcgagatggccgaggtccagctggtgcagtctggg
gctgaggtgaagaagcctgggtcctcggtgaaggtctcctgcaaggcttctggaggcaccttcagcagctatgctatcagctgg
gtgcgacaggcccctggacaagggcttgagtggatgggagggatcatccctatctttggtacagcaaactacgcacagaagttc
cagggcagagtcacgattaccgcggacgaatccacgagcacagcctacatggagctgagcagcctgagatctgaggacacg
gccgtgtattactgtgcgcgcaacaaccattactacaacgattactggggtcaaggtactctggtgaccgtctcctca [SEQ
ID NO: 639]
Amino Acid Sequence
NFMLTQPHSVSESPGKTVTISCTGSSGSIASNYVQWYQQRPGSAPTTVIYEDNQR
PSGVPDRFSGSIDSSSNSASLTISGLKTEDEADYYCQSYDSSNWVFGGGTKLTVL
GSRGGGGSGGGGSGGGGSLEMAEVQLVQSGAEVKKPGSSVKVSCKASGGTFSS
YAISWVRQAPGQGLEWMGGIIPIFGTANYAQKFQGRVTITADESTSTAYMELSSL
RSEDTAVYYCARNNHYYNDYWGQGTLVTVSS [SEQ ID NO: 640]

TABLE 101
ET200-027

DNA Sequence
cagtctgtgttgacgcagccgccctcagtgtctggggccccagggcagggggtcaccatcccctgcactgggagcagctcca
acatcggggcaggttatgatgtacactggtaccagcagcttccagggacagcccccaaactcctcatctatggtaacaacaatcg
gccctcaggggtccctgaccgcttctctggctccaggtctggctcctcagcctccctggccatcactgggctccaggctgaggat
gaggctgattattactgccagtcctatgacagcagcctgagtgatgtggtattcggcggagggaccaaggtcaccgtcctaggtt
ctagaggtggtggtggtagcggcggcggcggctctggtggtggtggatccctcgagatggccgaggtccagctggtgcagtct
ggggctgaggtgaagaagcctggggctacagtgaaaatctcctgcaaggtttctggatacaccttcaccgactactacatgcact
gggtgcaacaggcccctggaaaagggcttgagtggatgggacttgttgatcctgaagatggtgaaacaatatacgcagagaag
ttccagggcagagtcaccataaccgcggacacgtctacagacacagcctacatggagctgagcagcctgagatctgaggaca
cggccgtgtattactgtgcgcgctactggtcttactctttcgactacctgtacatgccggaaggtaacgattggtggggtcaaggta
ctctggtgaccgtctcctca [SEQ ID NO: 641]
Amino Acid Sequence
QSVLTQPPSVSGAPGQGVTIPCTGSSSNIGAGYDVHWYQQLPGTAPKLLIYGNNN
RPSGVPDRFSGSRSGSSASLAITGLQAEDEADYYCQSYDSSLSDVVFGGGTKVTV
LGSRGGGGSGGGGSGGGGSLEMAEVQLVQSGAEVKKPGATVKISCKVSGYTFT
DYYMHWVQQAPGKGLEWMGLVDPEDGETIYAEKFQGRVTITADTSTDTAYME
LSSLRSEDTAVYYCARYWSYSFDYLYMPEGNDWWGQGTLVTVSS [SEQ ID NO:
642]

TABLE 102
ET200-028

DNA Sequence
cagtctgtgttgactcagccacccgcagcgtctgggacccccggacagagagtcaccatctcttgttctgggggcgtctccaac
atcgggagtggtgctctaaattggtaccagcaactcccaggaacggcccccaaactcctcatctatagttacaatcagcggccct
caggggtctctgaccgattctctggctccaggtctgccacctcagcctccctggccatcagtgggctccagtctgaggatgaggc
tgattattactgtgcaacctgggatgatagtgtgaatggttgggtgttcggcggagggaccaagctgaccgtcctaggttctagag
gtggtggtggtagcggcggcggcggctctggtggtggtggatccctcgagatggcccaggtccagctggtacagtctggagct
gaggtgaagaagcctggggattcagtgaaggtctcctgcaagccttctggttacaattttctcaactatggtatcaactgggtgcg
acaggcccctggacaagggcttgagtggatgggatggattagcacttacaccggtaacacaaactatgcacagaagctgcagg
gcagagtcaccttcaccacagacacatccacgagcacagcctacatggagatgaggagcctgagatctgacgacacggccgt
gtattactgtgcgcgcgacctgtactactacgaaggtgttgattactggggtcaaggtactctggtgaccgtctcctca [SEQ
ID NO: 643]
Amino Acid Sequence
QSVLTQPPAASGTPGQRVTISCSGGVSNIGSGALNWYQQLPGTAPKLLIYSYNQR
PSGVSDRFSGSRSATSASLAISGLQSEDEADYYCATWDDSVNGWVFGGGTKLTV
LGSRGGGGSGGGGSGGGGSLEMAQVQLVQSGAEVKKPGDSVKVSCKPSGYNFL
NYGINWVRQAPGQGLEWMGWISTYTGNTNYAQKLQGRVTFTTDTSTSTAYME
MRSLRSDDTAVYYCARDLYYYEGVDYWGQGTLVTVSS [SEQ ID NO: 644]

TABLE 103
ET200-029

DNA Sequence
caggctgtgctgactcagccaccctcagtgtcagtggccccaggaaagacggccagggttacctgtgggggaaacaacattg
gaagtgaaagtgtgcactggtaccagcagaagccaggccaggcccctgtgttggtcatctattatgataccgaccggccctcag
ggatccctgagcgattctctggctcccactctgggaccacggccaccctgaccatcagcagggtcgaagccggggatgaggc
cgactattactgtcaggtgtgggatagtagtagggatcatgtggtattcggcggagggaccaagctgaccgtcctaggttctaga
ggtggtggtggtagcggcggcggcggctctggtggtggtggatccctcgagatggcccaggtgcagctggtgcagtctgggg
gaggcgtggtccagcctgggaggtccctgagactctcctgtgcggcctctggattcaccttcagtagctatgctatgcactgggt
ccgccaggctccaggcaagggactggagtgggtggcagttatatcatatgatggaagcaataaatactacgcagactccgtga
agggcctattcaccatctccagagacaattccaagaacacgctgtatctgcaaatgaacagcctgagagctgaggacacggcc
gtgtattactgtgcgcgctcttacttcacttctggtttctacgattactggggtcaaggtactctggtgaccgtctcctca [SEQ ID
NO: 645]
Amino Acid Sequence
QAVLTQPPSVSVAPGKTARVTCGGNNIGSESVHWYQQKPGQAPVLVIYYDTDRP
SGIPERFSGSHSGTTATLTISRVEAGDEADYYCQVWDSSRDHVVFGGGTKLTVLG
SRGGGGSGGGGSGGGGSLEMAQVQLVQSGGGVVQPGRSLRLSCAASGFTFSSY
AMHWVRQAPGKGLEWVAVISYDGSNKYYADSVKGLFTISRDNSKNTLYLQMN
SLRAEDTAVYYCARSYFTSGFYDYWGQGTLVTVSS [SEQ ID NO: 646]

TABLE 104
ET200-030

DNA Sequence
cagtctgtcgtgacgcagccgccctcagtgtctggggccccagggcagagggtcaccatctcctgcactgggagcagttccaa
catcggggcaggttatgatgtaaattggtatcagcagtttccaggaacagcccccaaactcctcatctatggtaacagcaatcggc
cctcaggggtccctgaccgattctctggctccaagtctggcacctcagcctccctggccatcactgggctccaggctgaggatg
aggctgattattactgccagtcctatgacagcagcctgagtggctcttatgtcttcggaactgggaccaaggtcaccgtcctaggtt
ctagaggtggtggtggtagcggcggcggcggctctggtggtggtggatccctcgagatggcccagatgcagctggtgcagtct
ggggctgaggtgaagaagcctggggcctcagtgaaggtctcctgcaaggcttccggatacaccctcactgaattatccatgcac
tgggtgcgacaggctcctggaaaagggcttgagtggatgggaggttttgatcctgaagatggtgaaacaatctacgcacagaag
ttccagggcagagtcaccatgaccgaggacacatctacagacacagcctacatggagctgagcagcctgagatctgaggaca
ctgccgtgtattactgtgcgcgcatgtcttctatgtactacgattggggtcaaggtactctggtgaccgtctcctca [SEQ ID
NO: 647]
Amino Acid Sequence
QSVVTQPPSVSGAPGQRVTISCTGSSSNIGAGYDVNWYQQFPGTAPKLLIYGNSN
RPSGVPDRFSGSKSGTSASLAITGLQAEDEADYYCQSYDSSLSGSYVFGTGTKVT
VLGSRGGGGSGGGGSGGGGSLEMAQMQLVQSGAEVKKPGASVKVSCKASGYT
LTELSMHWVRQAPGKGLEWMGGFDPEDGETIYAQKFQGRVTMTEDTSTDTAY
MELSSLRSEDTAVYYCARMSSMYYDWGQGTLVTVSS [SEQ ID NO: 648]

TABLE 105
ET200-031

DNA Sequence
tcctatgtgctgactcagccaccctcagtgtcagtggccccaggaaagacggccaggattacctgtgggggaaacaacattgga
agtaaaagtgtgcactggtaccagcagaagccaggccaggcccctgtgctggtcatctattatgatagcgaccggccctcagg
gatccctgagcgattctctggctccaactctgggaacacggccaccctgaccatcagcagggtcgaagccggggatgaggcc
gactattactgtcaggtgtgggatagtagtagtgattatgtcttcggaactgggaccaaggtcaccgtcctaggttctagaggtggt
ggtggtagcggcggcggcggctctggtggtggtggatccctcgagatggccgaggtgcagctggtggagactgggggaggc
ttggtcaagcctggagggtccctgagactctcctgtgcagcctctggattcaccgtcagtgactactacatgagctggatccgcca
ggctccagggaagggcctggagtggatttcatacattagtggtagtggtaatagcatatactacgcagactctgtgaagggccga
ttcaccatctccagggacaacgccaagaactcactggatctgcaaatgaccagcctgagagccgaggacacggccgtatatta
ctgtgcgcgctctactaaattcgattactggggtcaaggtactctggtgaccgtctcctca [SEQ ID NO: 649]
Amino Acid Sequence
SYVLTQPPSVSVAPGKTARITCGGNNIGSKSVHWYQQKPGQAPVLVIYYDSDRPS
GIPERFSGSNSGNTATLTISRVEAGDEADYYCQVWDSSSDYVFGTGTKVTVLGSR
GGGGSGGGGSGGGGSLEMAEVQLVETGGGLVKPGGSLRLSCAASGFTVSDYYM
SWIRQAPGKGLEWISYISGSGNSIYYADSVKGRFTISRDNAKNSLDLQMTSLRAE
DTAVYYCARSTKFDYWGQGTLVTVSS [SEQ ID NO: 650]

TABLE 106
ET200-032

DNA Sequence
ctgcctgtgctgactcagccaccctcagcgtctgggacccccgggcagagggtcaccatctcttgttctggaagcagctccaac
gtcggaagttacactgtaaactggtaccggcaactcccaggaacggcccccacactcctcatctataataataatcagcggccct
caggggtccctgaccgattctctgactccaagtctggcacctcggcctccctgaccattagtgggctccagcctgaggatgagg
ctgattattattgtgcagcatgggatgacaggctgggtggttatgtcttcggaactgggaccaaggtcaccgtcctaggttctaga
ggtggtggtggtagcggcggcggcggctctggtggtggtggatccctcgagatggccgaggtgcagctggtgcagtctggag
cagaggtgaaaaagccgggggagtctctgaagatctcctgtaagggttctggatacagctttaccaactactggatcggctgggt
gcgccagatgcccgggaaaggcctggagtggatggggatcatctatcctggtgactctgataccagatacagcccgtccttcca
aggccaggtcaccatctcagccgacaagtccatcagcaccgcctacctacagtggagcagcctgaaggcctcggacaccgcc
atgtattactgtgcgcgctctactggttcttctcatatgtctgatgaatggggtcaaggtactctggtgaccgtctcctca [SEQ
ID NO: 651]
Amino Acid Sequence
LPVLTQPPSASGTPGQRVTISCSGSSSNVGSYTVNWYRQLPGTAPTLLIYNNNQRP
SGVPDRFSDSKSGTSASLTISGLQPEDEADYYCAAWDDRLGGYVFGTGTKVTVL
GSRGGGGSGGGGSGGGGSLEMAEVQLVQSGAEVKKPGESLKISCKGSGYSFTNY
WIGWVRQMPGKGLEWMGIIYPGDSDTRYSPSFQGQVTISADKSISTAYLQWSSL
KASDTAMYYCARSTGSSHMSDEWGQGTLVTVSS [SEQ ID NO: 652]

TABLE 107
ET200-033

DNA Sequence
aattttatgctgactcagccccactctgtgtcggagtctccggggaagacggtaaccatctcctgcaccggcagcagtggcagc
attgccagcaactatgtgcagtggtaccagcagcgcccgggcagtgcccccaccactgtgatctatgaggataaccaaagacc
ctctggggtccctgatcggttctctggctccatcgacagctcctccaactctgcctccctcaccatctctggactgaagactgagg
acgaggctgactactactgtcagtcttatgatagcagcaatcattgggtgttcggcggagggaccaagctgaccgtcctaggttct
agaggtggtggtggtagcggcggcggcggctctggtggtggtggatccctcgagatggcccaagtgcagctacagcagtgg
ggcgcaggactgttgaagccttcggagaccctgtccctcacctgcgctgtctatggtgggtccttcagtggttactactggagctg
gatccgccagcccccagggaaggggctggagtggattggggagatcactcatagtggaaggtccaactacaacccgtccctc
aagagtcgagtcaccatatcagtagacacgtccaagaaccagttctccctgaagctgagctctgtgaccgccgcggacacggc
cgtgtattactgtgcgcgctcttctatcatgtctgattactggggtcaaggtactctggtgaccgtctcctca [SEQ ID NO:
653]
Amino Acid Sequence
NFMLTQPHSVSESPGKTVTISCTGSSGSIASNYVQWYQQRPGSAPTTVIYEDNQR
PSGVPDRFSGSIDSSSNSASLTISGLKTEDEADYYCQSYDSSNHWVFGGGTKLTV
LGSRGGGGSGGGGSGGGGSLEMAQVQLQQWGAGLLKPSETLSLTCAVYGGSFS
GYYWSWIRQPPGKGLEWIGEITHSGRSNYNPSLKSRVTISVDTSKNQFSLKLSSVT
AADTAVYYCARSSIMSDYWGQGTLVTVSS [SEQ ID NO: 654]

TABLE 108
ET200-034

DNA Sequence
cagtctgtgttgacgcagccgccctcagtgtctggggccccagggcagagggtcaccatctcctgcactgggagcacctccaa
catcggggcaggttatgatgtacactggtaccagcagcttccaggaacagcccccaaactcctcatcaacaataacaggaatcg
gccctcaggggtccctgaccgattctctggctccaagtctggcacgtcagccaccctgggcatcaccggactccagactgggg
acgaggccgattattactgcggaacatgggatggcagcctgactggtgcagtgttcggcggagggaccaagctgaccgtccta
ggttctagaggtggtggtggtagcggcggcggcggctctggtggtggtggatccctcgagatggccgaggtccagctggtgc
agtctggggctgaggtgaagaagcctgggtcctcggtgaaggtctcatgcaaggcttctggaggcaccttcagcagctatgcta
tcagctgggtgcgacaggcccctggacaagggcttgagtggatgggagggatcatccctatctttggtacagcaaactacgcac
agaagttccagggcagagtcacgattaccgcggacgaatccacgagcacagcctacatggagctgagcagcctgagatctga
ggacacggccgtgtattactgtgcgcgcggttctgctctggaccattacgatcgttggggtcaaggtactctggtgaccgtctcct
ca [SEQ ID NO: 655]
Amino Acid Sequence
QSVLTQPPSVSGAPGQRVTISCTGSTSNIGAGYDVHWYQQLPGTAPKLLINNNRN
RPSGVPDRFSGSKSGTSATLGITGLQTGDEADYYCGTWDGSLTGAVFGGGTKLT
VLGSRGGGGSGGGGSGGGGSLEMAEVQLVQSGAEVKKPGSSVKVSCKASGGTF
SSYAISWVRQAPGQGLEWMGGIIPIFGTANYAQKFQGRVTITADESTSTAYMELS
SLRSEDTAVYYCARGSALDHYDRWGQGTLVTVSS [SEQ ID NO: 656]

TABLE 109
ET200-035

DNA Sequence
aattttatgctgactcagccccactctgtgtcggagtctccggggaagacggtaaccatctcctgcacccgcagcagtggcagca
ttgccagcaactatgtgcagtggtaccagcagcgcccgggcagtgcccccaccactgtgatctatgaggataaccaaagaccct
ctggggtccctgatcggttctctggctccatcgacagctcctccaactctgcctccctcaccatctctggactgaagactgaggac
gaggctgactactactgtcagtcttatgatagcaccaattgggtgttcggcggagggaccaagctgaccgtcctaggttctagag
gtggtggtggtagcggcggcggcggctctggtggtggtggatccctcgagatggcccaggtgcagctggtgcagtctggggc
tgaggtgaagaagcctgggtcctcggtgaaggtctcctgcaaggcttctggaggcaccttcagcagctatgctatcagctgggt
gcgacaggcccctggacaagggcttgagtggatgggagggatcatccctatctttggtacagcaaactacgcacagaagttcc
agggcagagtcacgattaccgcggacgaatccacgagcacagcctacatggagctgagcagcctgagatctgaggacactgc
cgtgtattactgtgcgcgctacaactactacttcaacgattactggggtcaaggtactctggtgaccgtctcctca [SEQ ID
NO: 657]
Amino Acid Sequence
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSTNWVFGGGTKLTVLG
SRGGGGSGGGGSGGGGSLEMAQVQLVQSGAEVKKPGSSVKVSCKASGGTFSSY
AISWVRQAPGQGLEWMGGIIPIFGTANYAQKFQGRVTITADESTSTAYMELSSLR
SEDTAVYYCARYNYYFNDYWGQGTLVTVSS [SEQ ID NO: 658]

TABLE 110
ET200-037

DNA Sequence
tcctatgtgctgactcagccaccctcagtgtcagtggccccaggaaagacggccaggattacctgtgggggaaacaacattgga
agtaaaagtgtgcactggtaccagcagaagccaggccaggcccctgtgctggtcatctattatgatagcgaccggccctcagg
gatccctgagcgattctctggctccaactctgggaacacggccaccctgaccatcagcagggtcgaagccggggatgaggcc
gactattactgtcaggtgtgggatagtagtagtgatcatccttatgtcttcggaactgggaccaaggtcaccgtcctaggttctaga
ggtggtggtggtagcggcggcggcggctctggtggtggtggatccctcgagatggcccagatgcagctggtgcagtctggag
ctgaggtgaagaagcctggggcctcagtgaaggtctcctgcaaggcttctggttacacctttaccagctatggtatcagctgggt
gcgacaggcccctggacaagggcttgagtggatgggatggatcagcgcttacaatggtaacacaaactatgcacagaagctcc
agggcagagtcaccatgaccacagacacatccacgagcacagcctacatggagctgaggagcctgagatctgacgacactgc
cgtgtattactgtgcgcgctctatgttcggtgctcatgattcttggggtcaaggtactctggtgaccgtctcctca [SEQ ID
NO: 659]
Amino Acid Sequence
SYVLTQPPSVSVAPGKTARITCGGNNIGSKSVHWYQQKPGQAPVLVIYYDSDRPS
GIPERFSGSNSGNTATLTISRVEAGDEADYYCQVWDSSSDHPYVFGTGTKVTVLG
SRGGGGSGGGGSGGGGSLEMAQMQLVQSGAEVKKPGASVKVSCKASGYTFTS
YGISWVRQAPGQGLEWMGWISAYNGNTNYAQKLQGRVTMTTDTSTSTAYMEL
RSLRSDDTAVYYCARSMFGAHDSWGQGTLVTVSS [SEQ ID NO: 660]

TABLE 111
ET200-038

DNA Sequence
cagtctgtgttgacgcagccgccctcagtgtctggggccccagggcagagggtcaccatctcctgcactgggagcagctccaacatcggggca
ggttttgatgtacactggtaccagctacttccaggaacagcccccaaactcctcatctatgctaacagcaatcggccctcaggggtccctgaccga
ttctctggctccaagtctggcacctcagcctccctggccatcactgggctcctggctgaggatgaggctgattattactgccagtcctatgacagc
agcctgagtggtgtggtattcggcggagggaccaagctgaccgtcctaggttctagaggtggtggtggtagcggcggcggcggctctggtggtg
gtggatccctcgagatggcccaggtgcagctggtgcaatctggggctgaggtgaagaagcctgggtcctcggtgaaggtctcctgcaaggcttc
tggaggcaccttcagcagctatgctatcagctgggtgcgacaggcccctggacaagggcttgagtggatgggagggatcatccctatctttggta
cagcaaactacgcacagaagttccagggcagagtcacgattaccgcggacgaatccacgagcacagcctacatggagctgagcagcctgaga
tctgaggacactgccgtgtattactgtgcgcgcggtgcttctttcgaccgtcatgataactggggtcaaggtactctggtgaccgtctcctca
[SEQ ID NO: 661]
Amino Acid Sequence
QSVLTQPPSVSGAPGQRVTISCTGSSSNIGAGFDVHWYQLLPGTAPKLLIYANSNRPSGVPD
RFSGSKSGTSASLAITGLLAEDEADYYCQSYDSSLSGVVFGGGTKLTVLGSRGGGGSGGGG
SGGGGSLEMAQVQLVQSGAEVKKPGSSVKVSCKASGGTFSSYAISWVRQAPGQGLEWMG
GIIPIFGTANYAQKFQGRVTITADESTSTAYMELSSLRSEDTAVYYCARGASFDRHDNWGQG
TLVTVSS [SEQ ID NO: 662]

TABLE 112
ET200-039

DNA Sequence
aattttatgctgactcagccccactctgtgtcggagtctccggggaagacggtaaccatctcctgcacccgcagcagtggcagcattgccagcaa
ctatgtgcagtggtaccagcagcgcccgggcagttcccccaccactgtgatctatgaggataaccaaagaccctctggggtccctgatcggttct
ctggctccatcgacagctcctccaactctgcctccctcaccatctctggactgaagactgaggacgaggctgactactactgtcagtcttatgata
gcagcaattgggtgttcggcggagggaccaagctgaccgtcctaggttctagaggtggtggtggtagcggcggcggcggctctggtggtggtgg
atccctcgagatggccgaggtccagctggtgcagtctggggctgaggtgaagaagcctgggtcctcggtgaaggtctcctgcaaggcttctgga
ggcaccttcagcagctatgctatcagctgggtgcgacaggcccctggacaagggcttgagtggatgggagggatcatccctatctttggtacagc
aaactacgcacagaagttccagggcagagtcacgattaccgcggacgaatccacgagcacagcctacatggagctgagcagcctgagatctg
aggacacggccgtgtattactgtgcgcgctctaactactactacaacgattactggggtcaaggtactctggtgaccgtctcctca [SEQ ID
NO: 663]
Amino Acid Sequence
NFMLTQPHSVSESPGKTVTISCTRSSGSIASNYVQWYQQRPGSSPTTVIYEDNQRPSGVPDRF
SGSIDSSSNSASLTISGLKTEDEADYYCQSYDSSNWVFGGGTKLTVLGSRGGGGSGGGGSG
GGGSLEMAEVQLVQSGAEVKKPGSSVKVSCKASGGTFSSYAISWVRQAPGQGLEWMGGII
PIFGTANYAQKFQGRVTITADESTSTAYMELSSLRSEDTAVYYCARSNYYYNDYWGQGTL
VTVSS [SEQ ID NO: 664]

TABLE 113
ET200-040

DNA Sequence
cagtctgtgttgacgcagccgccctcagtgtctggggccccagggcagagggtcaccatctcctgcactgggagcagctccaacatcggggca
ggttatgatgtacactggtaccagcagcttccaggaacagcccccaaactcctcatctatggtaacagcaatcggccctcaggggtccctgaccg
attctctggctccaagtctggcacctcagcctccctggccatcactgggctccaggctgaggatgaggctgattattactgccagtcctatgacag
cagcctgagtggttatgtcttcggaactgggaccaaggtcaccgtcctaggttctagaggtggtggtggtagcggcggcggcggctctggtggtg
gtggatccctcgagatggcccaggtgcagctggtgcagtctggggctgaggtgaagaagcctggggcctcagtgaaggtctcctgcaaggtttc
cggatacaccctcactgaattatccatgcactgggtgcgacaggctcctggaaaagggcttgagtggatgggaggttttgatcctgaagatggtg
aaacaatctacgcacagaagttccagggcagagtcaccatgaccgaggacacatctacagacacagcctacatggagctgagcagcctgagat
ctgaggacactgccgtgtattactgtgcgcgctactctggtgtttactacgattggggtcaaggtactctggtgaccgtctcctca [SEQ ID
NO: 665]
Amino Acid Sequence
QSVLTQPPSVSGAPGQRVTISCTGSSSNIGAGYDVHWYQQLPGTAPKLLIYGNSNRPSGVPD
RFSGSKSGTSASLAITGLQAEDEADYYCQSYDSSLSGYVFGTGTKVTVLGSRGGGGSGGGG
SGGGGSLEMAQVQLVQSGAEVKKPGASVKVSCKVSGYTLTELSMHWVRQAPGKGLEWM
GGFDPEDGETIYAQKFQGRVTMTEDTSTDTAYMELSSLRSEDTAVYYCARYSGVYYDWGQ
GTLVTVSS [SEQ ID NO: 666]

TABLE 114
ET200-041

DNA Sequence
aattttatgctgactcagccccactctgtgtcggggtctccggggaagacggtaaccatctcctgcaccggcagcagtggcagcattgccgacaa
ctttgtgcagtggtaccagcagcgcccgggcggtgtccccaccactgtgatctttaatgatgacgaaagaccctctggcgtccctgatcggttctc
tggctccatcgacacctcctccaattctgcctccctcaccatctctggactgaagactgaggacgaggctgactactactgtcagtcttatgataa
taataatcgaggggtgttcggcggagggaccaagctgaccgtcctaggttctagaggtggtggtggtagcggcggcggcggctctggtggtggtgg
atccctcgagatggcccaggtccagctggtgcagtctggggctgaggtgaagaagcctgggtcctcggtgaaggtctcctgcaaggcttctgga
ggcaccttcagcagctatgctatcagctgggtgcgacaggcccctggacaagggcttgagtggatgggatggatgaaccctaacagtggtaaca
caggctatgcacagaagttccagggcagagtcaccatgaccaggaacacctccataagcacagcctacatggagctgagcaacctgagatctg
aggacacggccgtgtattactgtgcgcgctactactcttacggttacgattggggtcaaggtactctggtgaccgtctcctca [SEQ ID NO:
667]
Amino Acid Sequence
NFMLTQPHSVSGSPGKTVTISCTGSSGSIADNFVQWYQQRPGGVPTTVIFNDDERPSGVPDR
FSGSIDTSSNSASLTISGLKTEDEADYYCQSYDNNNRGVFGGGTKLTVLGSRGGGGSGGGGS
GGGGSLEMAQVQLVQSGAEVKKPGSSVKVSCKASGGTFSSYAISWVRQAPGQGLEWMGW
MNPNSGNTGYAQKFQGRVTMTRNTSISTAYMELSNLRSEDTAVYYCARYYSYGYDWGQG
TLVTVSS [SEQ ID NO: 668]

TABLE 115
ET200-042

DNA Sequence
cagtctgtcgtgacgcagccgccctcagtgtctggggccccagggcagacggtcaccatctcctgcactgggggcagctccaacatcgggaca
ggttattttgtaaattggtaccagcaggttccaggaaaagcccccaaactcctcatcctgggtaacaataatcggccctcgggggtccctgaccga
ctctccggctccacgtccggcacctcagcctccctggccatcactgggctccaggctgaggatgagggtacttattactgccagtcctatgacagc
agcctgagtggttatgtcttcggaactgggaccaaggtcaccgtcctaggttctagaggtggtggtggtagcggcggcggcggctctggtggtg
gtggatccctcgagatggcccaggtacagctgcagcagtcaggtccaggactggtgaagccctcgcagaccctctcactcacctgtggcatctc
cggggacagtgtctctaccaacagtgttgcttggcactggatcaggcagtccccatcgagaggccttgagtggctgggaaggacatactacagg
tccaagtggtctaatgactatggagtatctgtgaaaagtcgaatcaccatcatcccagacacatccaagaaccagttctccctgcagctgaactct
gtgactcccgaggacacggctgtgtattactgtgcgcgctcttcttcttggtaccagatcttcgattactggggtcaaggtactctggtgaccgtc
tcctca [SEQ ID NO: 669]
Amino Acid Sequence
QSVVTQPPSVSGAPGQTVTISCTGGSSNIGTGYFVNWYQQVPGKAPKLLILGNNNRPSGVPD
RLSGSTSGTSASLAITGLQAEDEGTYYCQSYDSSLSGYVFGTGTKVTVLGSRGGGGSGGGG
SGGGGSLEMAQVQLQQSGPGLVKPSQTLSLTCGISGDSVSTNSVAWHWIRQSPSRGLEWLG
RTYYRSKWSNDYGVSVKSRITIIPDTSKNQFSLQLNSVTPEDTAVYYCARSSSWYQIFDYWG
QGTLVTVSS [SEQ ID NO: 670]

TABLE 116
ET200-043

DNA Sequence
aattttatgctgactcagccccactctgtgtcggagtctccggggaagacggtaaccatctcctgcaccggcagcagcgacagcatagccaacaa
ctatgttcagtggtaccagcagcgcccgggcagtgcccccaccaatgtgatctacgaagatgtccaaagaccctctggggtccctgatcggttct
ctgggtccatcgacagctcctccaactctgcctccctcaccatctctggactgaagactgaggacgaggctgtctactattgtcagtcttatcata
gcgacaatcgttgggtgttcggcggcgggaccaagctgaccgtcctaggttctagaggtggtggtggtagcggcggcggcggctctggtggtggt
ggatccctcgagatggcccaggtgcagctggtggagtctgggggaggcttggtacagcctggggggtccctgagactctcctgtgcagcctctg
gattcacctttagcagctatgccatgagctgggtccgccaggctccagggaaggggctggagtgggtctcagctattagtggtagtggtggtagc
acatactacgcagactccgtgaagggccggttcaccatctccagagacaattccaagaacacgctgtatctgcaaatgaacagcctgagagccgag
gacacggccgtatattactgtgcgcgctctggtgcttactgggactactctgtttacgatgaatggggtcaaggtactctggtgaccgtctcctca
[SEQ ID NO: 671]
Amino Acid Sequence
NFMLTQPHSVSESPGKTVTISCTGSSDSIANNYVQWYQQRPGSAPTNVIYEDVQRPSGVPDR
FSGSIDSSSNSASLTISGLKTEDEAVYYCQSYHSDNRWVFGGGTKLTVLGSRGGGGSGGGGS
GGGGSLEMAQVQLVESGGGLVQPGGSLRLSCAASGFTFSSYAMSWVRQAPGKGLEWVSAI
SGSGGSTYYADSVKGRFTISRDNSKNTLYLQMNSLRAEDTAVYYCARSGAYWDYSVYDE
WGQGTLVTVSS [SEQ ID NO: 672]

TABLE 117
ET200-044

DNA Sequence
cagtctgtgttgactcagccaccctcagtgtccgtgtccccaggacagacagccaccatcgcctgttctggacataaattgggggataaatatgct
tcctggtatcagcagaagtcgggccagtcccctgtgttgatcatctatcaggataataagcggccctcagggattcctgagcgattctctggctcc
aactctgggaacacagccactctgaccatcagcgggacccaggctctggatgaggctgactattattgtcaggcgtgggacagtagtacttatgtg
gcattcggcggagggaccaagctgaccgtcctaggttctagaggtggtggtggtagcggcggcggcggctctggtggtggtggatccctcgaga
tggcccaggtgcagctgcaggagtccggcccaggactggtgaagccttcggagaccctgtccctcacctgcgttgtctctggtggctccatcag
cagtagtaactggtggagctgggtccgccagcccccagggaaggggctggagtggattggggaaatctatcatagtgggagccccaactacaa
cccatccctcaagagtcgagtcaccatatcagtagacaagtccaagaaccagttctccctgaagctgagctctgtgaccgccgcggacacggcc
gtgtattactgtgcgcgcatgactactcatactttcggttacgatgcttggggtcaaggtactctggtgaccgtctcctca [SEQ ID NO:
673]
Amino Acid Sequence
QSVLTQPPSVSVSPGQTATIACSGHKLGDKYASWYQQKSGQSPVLIIYQDNKRPSGIPERFSG
SNSGNTATLTISGTQALDEADYYCQAWDSSTYVAFGGGTKLTVLGSRGGGGSGGGGSGGG
GSLEMAQVQLQESGPGLVKPSETLSLTCVVSGGSISSSNWWSWVRQPPGKGLEWIGEIYHS
GSPNYNPSLKSRVTISVDKSKNQFSLKLSSVTAADTAVYYCARMTTHTFGYDAWGQGTLV
TVSS [SEQ ID NO: 674]

TABLE 118
ET200-045

DNA Sequence
cagcctgtgctgactcagccaccctcagtgtcagtggccccaggaaagacggccacgattacttgtgggggaaacaacattggaagtgaaagtg
tgcactggtaccaccagaagccaggccaggcccctgtgttggtcatctatgatgatgccggccggccctcagggatccctgagcgattcactgg
ctccaactctgggaacacggccaccctgaccatcagcagggtcgaagccggggatgaggccgactattactgtcaggtgtgggacagaaatag
tgctcagtttgtcttcggacctgggaccaaggtcaccgtcctaggttctagaggtggtggtggtagcggcggcggcggctctggtggtggtggat
ccctcgagatggccgaggtccagctggtgcagtctggagctgaggtgaagaagcctggggcctcagtgaaggtctcctgcaaggcttctggtta
cacctttaccagctatggtatcagctgggtgcgacaggcccctggacaagggcttgagtggatgggatggatcagcgcttacaatggtaacacaa
actatgcacagaagctccagggcagagtcaccatgaccacagacacatccacgagcacagcctacatggagctgaggagcctgagatctgac
gacacggccgtgtattactgtgcgcgcggtgttcatctggattggtggggtcaaggtactctggtgaccgtctcctca [SEQ ID NO: 675]
Amino Acid Sequence
QPVLTQPPSVSVAPGKTATITCGGNNIGSESVHWYHQKPGQAPVLVIYDDAGRPSGIPERFT
GSNSGNTATLTISRVEAGDEADYYCQVWDRNSAQFVFGPGTKVTVLGSRGGGGSGGGGSG
GGGSLEMAEVQLVQSGAEVKKPGASVKVSCKASGYTFTSYGISWVRQAPGQGLEWMGWI
SAYNGNTNYAQKLQGRVTMTTDTSTSTAYMELRSLRSDDTAVYYCARGVHLDWWGQGT
LVTVSS [SEQ ID NO: 676]

TABLE 119
ET200-069

DNA Sequence
cagtctgtcgtgacgcagccaccctcagcgtctgggacccccgggcagagggtcaccatctcttgttctggaagcagctccaacatcggaagta
attatgtatactggtaccagcagctcccaggaacggcccccaaactcctcatctatagtaataatcagcggccctcaggggtccctgaccgattct
ctggctccaagtctggcacctcagcctccctggccatcagtgggctccggtccgaggatgaggctgattattactgtgcagcatgggatgacagcc
tgagtggttatgtcttcggaactgggaccaagctgaccgtcctaggttctagaggtggtggtggtagcggcggcggcggctctggtggtggtgga
tccctcgagatggcccaggtgcagctacagcagtggggcgcaggactgttgaagccttcggagaccctgtccctcacctgcgctgtctatggtg
ggtccttcagtggttactactggagctggatccgccagcccccagggaaggggctggagtggattggggaaatcaatcatagtggaagcacca
actacaacccgtccctcaagagtcgagtcaccatatcagtagacacgtccaagaaccagttctccctgaagctgagctctgtgaccgccgcggac
acggccgtgtattactgtgcgcgcctgtacgaaggtggttaccatggttggggttcttggctgtcttctgattcttggggtcaaggtactctggtg
accgtctcctca [SEQ ID NO: 677]
Amino Acid Sequence
QSVVTQPPSASGTPGQRVTISCSGSSSNIGSNYVYWYQQLPGTAPKLLIYSNNQRPSGVPDRF
SGSKSGTSASLAISGLRSEDEADYYCAAWDDSLSGYVFGTGTKLTVLGSRGGGGSGGGGSG
GGGSLEMAQVQLQQWGAGLLKPSETLSLTCAVYGGSFSGYYWSWIRQPPGKGLEWIGEIN
HSGSTNYNPSLKSRVTISVDTSKNQFSLKLSSVTAADTAVYYCARLYEGGYHGWGSWLSSD
SWGQGTLVTVSS [SEQ ID NO: 678]

TABLE 120
ET200-078

DNA Sequence
cagtctgtgttgactcagccaccctcagcgtctgggacccccgggcagagggtcaccatctcttgttctggaagcagctccaacatcggaagtaa
tactgtaaactggtaccagcagctcccaggaacggcccccaaactcctcatctatagtaataatcagcggccctcaggggtccctgaccgattctc
tggctccaagtctggcacctcagcctccctggccatcagtgggctccagtctgaggatgaggctgattattactgtgcagcatgggatgacagcct
gaatggttattgggtgttcggcggagggaccaagctgaccgtcctaggttctagaggtggtggtggtagcggcggcggcggctctggtggtggt
ggatccctcgagatggcccaggtgcagctacagcagtggggcgcaggactgttgaagccttcggagaccctgtccctcacctgcgctgtctatg
gtgggtccttcagtggttactactggagctggatccgccagcccccagggaaggggctggagtggattggggaaatcaatcatagtggaagcac
caactacaacccgtccctcaagagtcgagtcaccatatcagtagacacgtccaagaaccagttctccctgaagctgagctctgtgaccgccgcgg
acacggctgtgtattactgtgcgcgcgaaggggcatttgatgcttttgatatctggggccaagggacaatggtcaccgtctcttca [SEQ ID
NO: 679]
Amino Acid Sequence
QSVLTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYWVFGGGTKLTVLGSRGGGGSGGGG
SGGGGSLEMAQVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCAREGAFDAFDIWGQGT
MVTVSS [SEQ ID NO: 680]

TABLE 121
ET200-079

DNA Sequence
tcctatgagctgactcagccaccctcagcgtctgggacccccgggcagagggtcaccatctcttgttctggaagcagctccaacatcggaagtaa
ttatgtatactggtaccagcagctcccaggaacggcccccaaactcttcatctataggaataatcagcggccctcaggggtccctgaccgattctc
tggctccaagtctggcacctcagcctccctggccatcagtgggctccggtccgaggatgaggctgattattactgtgcagcatgggatgacagcct
gagtggttatctcttcggaactgggaccaaggtcaccgtcctaggttctagaggtggtggtggtagcggcggcggcggctctggtggtggtggat
ccctcgagatggccgaggtgcagctggtggagtctgggggaggcttggtacagcctggcaggtccctgagactctcctgtgcagcctctggatt
cacctttgatgattatgccatgcactgggtccggcaagctccagggaagggcctggagtgggtctcaggtattagttggaatagtggtagcatagg
ctatgcggactctgtgaagggccgattcaccatctccagagacaacgccaagaactccctgtatctgcaaatgaacagtctgagagctgaggaca
cggccttgtattactgtgcaaatggcgactccaactactactacggtatggacgtctggggccaagggaccacggtcaccgtctcctca [SEQ
ID NO: 681]
Amino Acid Sequence
SYELTQPPSASGTPGQRVTISCSGSSSNIGSNYVYWYQQLPGTAPKLFIYRNNQRPSGVPDRF
SGSKSGTSASLAISGLRSEDEADYYCAAWDDSLSGYLFGTGTKVTVLGSRGGGGSGGGGSG
GGGSLEMAEVQLVESGGGLVQPGRSLRLSCAASGFTFDDYAMHWVRQAPGKGLEWVSGIS
WNSGSIGYADSVKGRFTISRDNAKNSLYLQMNSLRAEDTALYYCANGDSNYYYGMDVWG
QGTTVTVSS [SEQ ID NO: 682]

TABLE 122
ET200-081

DNA Sequence
cagtctgccctgactcagcctgcctccgtgtccgggtctcctggacagtcgatcaccatctcctgcactggaaccagcagtgacattggtggttat
aactatgtctcctggtaccaacaacacccaggcaaagcccccaaactcatgatttatgatgtcagtaatcggccctcaggggtttctaatcgcttc
tctggctccaagtctggcaacacggcctccctgaccatctctgggctccaggctgaggacgaggctgattattactgcatctcatatacacgcacc
tggaacccctatgtcttcgggagtgggaccaaggtcaccgtcctaggttctagaggtggtggtggtagcggcggcggcggctctggtggtggtgga
tccctcgagatggccgaggtgcagctggtgcagtctgggggaggcgtggtacagcctggggggtccctgagactctcctgtgcagcctctggatt
cacctttgatgattatgccatgcactgggtccgtcaagctccagggaagggtctggagtgggtctctcttattagtggggatggtggtagcacata
ctatgcagactctgtgaagggccgattcaccatctccagagacaacagcaaaaactccctgtatctgcaaatgaacagtctgagaactgaggacac
cgccttgtattactgtgcaaaagatcgggcagcagctggctactactactacggtatggacgtctggggccaagggaccacggtcaccgtctcct
ca [SEQ ID NO: 683]
Amino Acid Sequence
QSALTQPASVSGSPGQSITISCTGTSSDIGGYNYVSWYQQHPGKAPKLMIYDVSNRPSGVSN
RFSGSKSGNTASLTISGLQAEDEADYYCISYTRTWNPYVFGSGTKVTVLGSRGGGGSGGGG
SGGGGSLEMAEVQLVQSGGGVVQPGGSLRLSCAASGFTFDDYAMHWVRQAPGKGLEWVS
LISGDGGSTYYADSVKGRFTISRDNSKNSLYLQMNSLRTEDTALYYCAKDRAAAGYYYYG
MDVWGQGTTVTVSS [SEQ ID NO: 684]

TABLE 123
ET200-097

DNA Sequence
ctgcctgtgctgactcagccaccctcagtgtccgtgtccccaggacagacagccatcatcacctgctctggagataaattgggggaaaaatatgtt
tcctggtatcagcagaagccaggccagtcccctgtactggtcatcgatcaagataccaggaggccctcagggatccctgagcgattctctggctc
caactctgggaccacagccactctgaccatcagcgggacccaggctatggatgaggctgactattactgtcaggcgtgggacaggggtgtggta
ttcggcggagggaccaagctgaccgtcctaggttctagaggtggtggtggtagcggcggcggcggctctggtggtggtggatccctcgagatg
gccgaggtgcagctggtggagtctgggggagacttggtacagcctggcaggtccctgagactctcctgtgcagcctctggattcacctttaatgat
tatgccatgcactgggtccggcaagctccagggaagggcctggagtgggtctcaggtattagttggagtggtaataacataggctatgcggactc
tgtgaagggccgattcaccatctccagagacaacgccaagaactccctgtatctgcaaatgaacagtctgagagctgaggacacggccttgtatt
actgtgcaaaagatagtatacggtatggcatcacctggggaggttttgactactggggccagggaaccctggtcaccgtctcctca [SEQ ID
NO: 685]
Amino Acid Sequence
LPVLTQPPSVSVSPGQTAIITCSGDKLGEKYVSWYQQKPGQSPVLVIDQDTRRPSGIPERFSG
SNSGTTATLTISGTQAMDEADYYCQAWDRGVVFGGGTKLTVLGSRGGGGSGGGGSGGGG
SLEMAEVQLVESGGDLVQPGRSLRLSCAASGFTFNDYAMHWVRQAPGKGLEWVSGISWS
GNNIGYADSVKGRFTISRDNAKNSLYLQMNSLRAEDTALYYCAKDSIRYGITWGGFDYWG
QGTLVTVSS [SEQ ID NO: 686]

TABLE 124
ET200-098

DNA Sequence
cagcctgtgctgactcagccaccctcggtgtccaagggcttgagacagaccgccacactcacctgcactgggaacagcaacaatgttggcaac
ctaggagtagcttggctgcagcagcaccagggccaccctcccaaactcctatcctacaggaataacaaccggccctcagggatctcagagagat
tatctgcatccaggtcaggaaacacagcctccctgaccattactggactccagcctgaggacgaggctgactattactgctcagcatgggacagt
agcctcagtgcttgggtgttcggcggagggaccaagctgaccgtcctaggttctagaggtggtggtggtagcggcggcggcggctctggtggt
ggtggatccctcgagatggccgaggtgcagctggtggagtctgggggagtcgtggtacagcctggggggtccctgagactctcctgtgcagcctc
tggattcacctttgatgattatgccatgcactgggtccgtcaagctccggggaagggtctggagtgggtctctcttattaattgggatggtggtag
cacctactatgcagactctgtgaagggtcgattcaccatctccagagacaacagcaaaaactccctgtatctgcaaatgaacagtctgagagctga
ggacaccgccttgtattactgtgcaaaagggatgggcctgagggcgtttgactactggggccagggaaccctggtcaccgtctcctca [SEQ
ID NO: 687]
Amino Acid Sequence
QPVLTQPPSVSKGLRQTATLTCTGNSNNVGNLGVAWLQQHQGHPPKLLSYRNNNRPSGISE
RLSASRSGNTASLTITGLQPEDEADYYCSAWDSSLSAWVFGGGTKLTVLGSRGGGGSGGGG
SGGGGSLEMAEVQLVESGGVVVQPGGSLRLSCAASGFTFDDYAMHWVRQAPGKGLEWVS
LINWDGGSTYYADSVKGRFTISRDNSKNSLYLQMNSLRAEDTALYYCAKGMGLRAFDYW
GQGTLVTVSS [SEQ ID NO: 688]

TABLE 125
ET200-099

DNA Sequence
cagtctgtgttgactcagccaccctcagcgtctgggacccccgggcagagggtcaccatctcctgttctggaagcagctccaacatcggaagtaa
tactgtaaactggtaccagcagctcccaggaacggcccccaaactcctcatctatagtaatgatcagcggccctcaggggtccctgaccgattctc
tggctccaagtccggcacctcagcctccctggccatcagtgggctccagtctgaggatgaggctgattattactgtgcttcatgggatgacagcct
gaatggccgttatgtcttcggaactgggaccaaggtcaccgtcctaggttctagaggtggtggtggtagcggcggcggcggctctggtggtggt
ggatccctcgagatggcccaggtccagctggtacagtctggggctgaggtgaggaagcctggggcctcagtgaaggtttcctgcaagacttctg
gatacaccttcagttggtatgctatacattgggtgcgccaggcccccggacaaaggcttgagtggatgggatggatcaacgctggcaatggaaa
cacaaaatattcacagaaatttcagggcagagtcagtcttaccagggacacatccgcgagcacagcctacatggagctgagcagcctgagatct
gatgacacggctgtgtattactgtgcgagacccgataattatggttcgggtggggatgtttttgatatctggggccaagggacaatggtcaccgtc
tcttca [SEQ ID NO: 689]
Amino Acid Sequence
QSVLTQPPSASGTPGQRVTISCSGSSSNIGSNTVNWYQQLPGTAPKLLIYSNDQRPSGVPDRF
SGSKSGTSASLAISGLQSEDEADYYCASWDDSLNGRYVFGTGTKVTVLGSRGGGGSGGGGS
GGGGSLEMAQVQLVQSGAEVRKPGASVKVSCKTSGYTFSWYAIHWVRQAPGQRLEWMG
WINAGNGNTKYSQKFQGRVSLTRDTSASTAYMELSSLRSDDTAVYYCARPDNYGSGGDVF
DIWGQGTMVTVSS [SEQ ID NO: 690]

TABLE 126
ET200-100

DNA Sequence
aattttatgctgactcagccccactctgtgtcggagtctccggggaagacggtaaccatctcctgcacccgcagcagtggcagca
ttgccagcaactttgtgcagtggtaccagcagcgcccgggcagtgcccccacccctatgatctatgaggataacaacagacccc
ctggggtccctgatcggttctctgcctccgtcgacagctcctccaactctgcctccctcaccatctctggactgaagactgaggac
gaggctgactactactgtcagtcttatgataccagcaatgtggtattcggcggggggaccaagctgaccgtcctaggttctagag
gtggtggtggtagcggcggcggcggctctggtggtggtggatccctcgagatggccgaggtgcagctggtggagtctggggg
aggcttggtacagcctggagggtccctgagactctcctgtgcagcctctggattcaccttcagtagttatgaaatgaactgggtcc
gccaggctccagggaaggggctggagtgggtttcatacattagtagtagtggtagtaccatatactacgcagactctgtgaagg
gccgattcaccatctccagagacaacgccaagaactcactgtatctgcaaatgaacagcctgagagccgaggacacggctgttt
attactgtgcacgctgggactacggtatggacgtctggggccaagggaccacggtcaccgtctcctca [SEQ ID NO:
691]
Amino Acid Sequence
NFMLTQPHSVSESPGKTVTISCTRSSGSIASNFVQWYQQRPGSAPTPMIYEDNNRP
PGVPDRFSASVDSSSNSASLTISGLKTEDEADYYCQSYDTSNVVFGGGTKLTVLG
SRGGGGSGGGGSGGGGSLEMAEVQLVESGGGLVQPGGSLRLSCAASGFTFSSYE
MNWVRQAPGKGLEWVSYISSSGSTIYYADSVKGRFTISRDNAKNSLYLQMNSLR
AEDTAVYYCARWDYGMDVWGQGTTVTVSS [SEQ ID NO: 692]

TABLE 127
ET200-101

DNA Sequence
caggctgtgctgactcagccaccctcagcgtctggggcccccgggcagagggtcaccgtctcttgttctggaagcaactccaac
atcggaagtaactacgttaactggtaccagcagttcccaggaacggcccccaaactcctcatgtatagtagtagtcagcggccct
caggggtccctgaccgattctctggctccaagtctggcacctcagcctccctggccatcagtgggctccactctgaggatgagg
ctgattattactgtgctacatgggatgacagcctgaatgcttgggtgttcggcggagggaccaagctgaccgtcctaggttctaga
ggtggtggtggtagcggcggcggcggctctggtggtggtggatccctcgagatggccgaggtccagctggtgcagtctgggg
ctgaggtgaggaagcctggggcctcagtgaaggtttcctgcaagacttctggatacaccttcacttggtatgctatacattgggtg
cgccaggcccccggacaaaggcttgagtggatgggatggatcaacgctggcagtggaaacacaaaatattcacagaaatttca
gggcagagtcacccttaccagggacacatccgcgagcacagcgtacatggagctgagcagcctgagatctgatgacacggct
gtgtattactgtgcgagacccaataactatggttcgggtggggatgtttttgatatctggggccaagggacaatggtcaccgtctct
tca [SEQ ID NO: 693]
Amino Acid Sequence
QAVLTQPPSASGAPGQRVTVSCSGSNSNIGSNYVNWYQQFPGTAPKLLMYSSSQ
RPSGVPDRFSGSKSGTSASLAISGLHSEDEADYYCATWDDSLNAWVFGGGTKLT
VLGSRGGGGSGGGGSGGGGSLEMAEVQLVQSGAEVRKPGASVKVSCKTSGYTF
TWYAIHWVRQAPGQRLEWMGWINAGSGNTKYSQKFQGRVTLTRDTSASTAYM
ELSSLRSDDTAVYYCARPNNYGSGGDVFDIWGQGTMVTVSS [SEQ ID NO: 694]

TABLE 128
ET200-102

DNA Sequence
cagtctgtcgtgacgcagccgccctcagtgtctgcggccccaggacagaaggtcaccatctcctgctctggaagcagctccaa
cattgggaataattatgtatcctggtaccagcagctcccaggaacagcccccaaactcctcatttatgacaataataagcgaccct
cagggattcctgaccgattctctggctccaagtctggcacgtcagccaccctgggcatcaccggactccagactggggacgag
gccgattattactgcggaacatgggatagcagcctgagtgcttatgtcttcggaactgggaccaaggtcaccgtcctaggttctag
aggtggtggtggtagcggcggcggcggctctggtggtggtggatccctcgagatggcccaggtccagctggtgcagtctggg
gctgaggtgaagaagcctggggcctcagtgaaagtttcctgcaaggcttctggatacaccttcacgaactatgctctgcattgggt
gcgccaggcccccggacaagggcttgagtggatggcatggatcaacggtggcaatggtaacacaaaatattcacagaacttcc
agggcagagtcaccattaccagggacacatccgcgagcacagcctatatggagctgagcagcctgagatctgaagacacggc
tgtgtattactgtgcgaaaccggaggaaacagctggaacaatccactttgactactggggccagggaaccccggtcaccgtctc
ctca [SEQ ID NO: 695]
Amino Acid Sequence
QSVVTQPPSVSAAPGQKVTISCSGSSSNIGNNYVSWYQQLPGTAPKLLIYDNNKR
PSGIPDRFSGSKSGTSATLGITGLQTGDEADYYCGTWDSSLSAYVFGTGTKVTVL
GSRGGGGSGGGGSGGGGSLEMAQVQLVQSGAEVKKPGASVKVSCKASGYTFT
NYALHWVRQAPGQGLEWMAWINGGNGNTKYSQNFQGRVTITRDTSASTAYME
LSSLRSEDTAVYYCAKPEETAGTIHFDYWGQGTPVTVSS [SEQ ID NO: 696]

TABLE 129
ET200-103

DNA Sequence
caggctgtgctgactcagccccactctgtgtcggagtctccggggaagacggtaaccatctcctgcacccgcagcagtggcag
cattgccagcaactatgtgcagtggtaccagcagcgcccgggcagtgcccccaccactgtgatctatgaggataaccaaagac
cctctggggtccctgatcggttctctggctccatcgacagctcctccaactctgcctccctcaccatctctggactgaagactgag
gacgaggctgactactactgtcagtcttatgatagcaccatcacggtgttcggcggagggaccaagctgaccgtcctaggttcta
gaggtggtggtggtagcggcggcggcggctctggtggtggtggatccctcgagatggcccaggtccagctggtacagtctgg
ggctgaggtgaagaagcctgggtcctcggtgaaggtctcctgcaaggcttctggaggcaccttcagcagctatgctatcagctg
ggtgcgacaggcccctggacaagggcttgagtggatgggagggatcatccctatctttggtacagcaaactacgcacagaagtt
ccagggcagagtcacgattaccgcggacgaatccacgagcacagcctacatggagctgagcagcctgagatctgaggacac
ggccgtgtattactgtgcgggggagggttactatgatagtagtggttattccaacggtgatgcttttgatatctggggccaaggga
caatggtcaccgtctcttca [SEQ ID NO: 697]
Amino Acid Sequence
QAVLTQPHSVSESPGKTVTISCTRSSGSIASNYVQWYQQRPGSAPTTVIYEDNQRP
SGVPDRFSGSIDSSSNSASLTISGLKTEDEADYYCQSYDSTITVFGGGTKLTVLGS
RGGGGSGGGGSGGGGSLEMAQVQLVQSGAEVKKPGSSVKVSCKASGGTFSSYA
ISWVRQAPGQGLEWMGGIIPIFGTANYAQKFQGRVTITADESTSTAYMELSSLRS
EDTAVYYCAGEGYYDSSGYSNGDAFDIWGQGTMVTVSS [SEQ ID NO: 698]

TABLE 130
ET200-104

DNA Sequence
aattttatgctgactcagccccactctgtgtcggagtctccggggaagacggtaaccatctcctgcacccgcagcagtggcagca
ttgccagcaactatgtgcagtggtaccagcagcgcccgggcagtgcccccaccactgtgatctatgaggataaccaaagaccct
ctggggtccctgatcggttctctggctccatcgacagctcctccaactctgcctccctcaccatctctggactgaagactgaggac
gaggctgactactactgtcagtcttatgatagcagcaatgtggtattcggcggagggaccaaggtcaccgtcctaggttctagag
gtggtggtggtagcggcggcggcggctctggtggtggtggatccctcgagatggccgaggtgcagctggtggagtctggggg
aggcttggtacagcctggagggtccctgagactctcctgtgcagcctctggattcaccttcagtagttatgaaatgaactgggtcc
gccaggctccagggaaggggctggagtgggtttcatacattagtagtagtggtagtaccatatactacgcagactctgtgaagg
gccgattcaccatctccagagacaacgccaagaactcactgtatctgcaaatgaacagcctgagagccgaggacacggctgttt
attactgtgcacgctgggactacggtatggacgtctggggccaagggaccacggtcaccgtctcctca [SEQ ID NO:
699]
Amino Acid Sequence
NFMLTQPHSVSESPGKTVTISCTRSSGSIASNYVQWYQQRPGSAPTTVIYEDNQRP
SGVPDRFSGSIDSSSNSASLTISGLKTEDEADYYCQSYDSSNVVFGGGTKVTVLGS
RGGGGSGGGGSGGGGSLEMAEVQLVESGGGLVQPGGSLRLSCAASGFTFSSYE
MNWVRQAPGKGLEWVSYISSSGSTIYYADSVKGRFTISRDNAKNSLYLQMNSLR
AEDTAVYYCARWDYGMDVWGQGTTVTVSS [SEQ ID NO: 700]

TABLE 131
ET200-105

DNA Sequence
tcctatgtgctgactcagccaccctcagtgtccgtgtccccaggacagacagccagcatcacctgctctggagatagattgacga
ataaatatgtttcctggtatcaacagaagccaggccagtcccctgtgttggtcatctatgaggatgccaagcggccctcagggatc
cctgcgcgattctctggctccaactctgggaacacagccactctgaccatcagcgggacccaggctatggatgagtctgaatatt
actgtcaggcgtgggacagcagtgtggtggtttttggcggagggaccaagctgaccgtcctaggttctagaggtggtggtggta
gcggcggcggcggctctggtggtggtggatccctcgagatggccgaggtgcagctggtggagtctgggggaggcttggtaca
gcctggcaggtccctgagactctcctgtgcagcctctggatttacctttgatgattatgccatgcactgggtccggcaagctccag
ggaagggcctggagtgggtctcaggtattagttggaatagtggtagtataggctatgcggactctgtgaagggccgattcaccat
ctccagagacaacgccaagaactccctgtatctgcaaatgaacagtctgagagatgaggacacggccttgtattactgtgcaaaa
gaccgaggggggggagttatcgttaaggatgcttttgatatctggggccaagggacaatggtcaccgtctcttca [SEQ ID
NO: 701]
Amino Acid Sequence
SYVLTQPPSVSVSPGQTASITCSGDRLTNKYVSWYQQKPGQSPVLVIYEDAKRPS
GIPARFSGSNSGNTATLTISGTQAMDESEYYCQAWDSSVVVFGGGTKLTVLGSR
GGGGSGGGGSGGGGSLEMAEVQLVESGGGLVQPGRSLRLSCAASGFTFDDYAM
HWVRQAPGKGLEWVSGISWNSGSIGYADSVKGRFTISRDNAKNSLYLQMNSLR
DEDTALYYCAKDRGGGVIVKDAFDIWGQGTMVTVSS [SEQ ID NO: 702]

TABLE 132
ET200-106

DNA Sequence
tcctatgagctgactcagccacccgcagcgtctgggacccccggacagagagtcaccatctcttgttctgggggcgtctccaac
atcgggagtggtgctctaaattggtaccagcaactcccaggaacggcccccaaactcctcatctatagttacaatcagcggccct
caggggtctctgaccgattctctggctccaggtctgccacctcagcctccctggccatcagtgggctccagtctgaggatgaggc
tgattattactgtgcaacctgggatgatagtgtgaatggttgggtgttcggcggagggaccaagctgaccgtcctaggttctagag
gtggtggtggtagcggcggcggcggctctggtggtggtggatccctcgagatggccgaggtgcagctggtggagtctggagc
tgaggtgaagaagcctggggattcagtgaaggtctcctgcaagccttctggttacaattttctcaactatggtatcaactgggtgcg
acaggcccctggacaagggcttgagtggatgggatggattagcacttacaccggtaacacaaactatgcacagaagctgcagg
gcagagtcaccttcaccacagacacatccacgagcacagcctacatggagatgaggagcctgagatctgacgacacggccgt
gtattactgtgcgcgccagcagggtggtggttggtacgatgtttggggtcaaggtactctggtcaccgtctcctca [SEQ ID
NO: 703]
Amino Acid Sequence
SYELTQPPAASGTPGQRVTISCSGGVSNIGSGALNWYQQLPGTAPKLLIYSYNQR
PSGVSDRFSGSRSATSASLAISGLQSEDEADYYCATWDDSVNGWVFGGGTKLTV
LGSRGGGGSGGGGSGGGGSLEMAEVQLVESGAEVKKPGDSVKVSCKPSGYNFL
NYGINWVRQAPGQGLEWMGWISTYTGNTNYAQKLQGRVTFTTDTSTSTAYME
MRSLRSDDTAVYYCARQQGGGWYDVWGQGTLVTVSS [SEQ ID NO: 704]

TABLE 133
ET200-107

DNA Sequence
cagtctgtcgtgacgcagccgccctcagtgtctgcggccccaggagagaaggtcaccatctcctgctctggaagcaacttcaat
gttggaaataatgatgtatcctggtatcagcaactcccaggtgcagcccccaaactcctcatttatgacaataataagcgaccctca
gggattcctgaccgattctctggctccaagtctggcacgtcagccaccctggacatcaccgggctccacagtgacgacgaggc
cgattattactgcggaacatgggatagcagcctgaatactgggggggtcttcggaactgggaccaaggtcaccgtcctaggttct
agaggtggtggtggtagcggcggcggcggctctggtggtggtggatccctcgagatggccgaggtccagctggtgcagtctg
gagctgaggtgaagaagcctggggcctcagtgaaggtctcctgcaaggcttctggttacacctttaccagctatactatcagctg
ggtacgacaggcccctggacaagggcttgagtggatgggatggatcagcacttacaatggtctcacaaactatgcacagaacct
ccagggcagagtcaccatgactacagacacattcacgaccacagcctacatggagctgaggagcctcagatctgacgacacg
gccgtgtattactgtgtgagagaggggtcccccgactacggtgacttcgcgtcctttgactactggggccagggaaccctggtca
ccgtctcctca [SEQ ID NO: 705]
Amino Acid Sequence
QSVVTQPPSVSAAPGEKVTISCSGSNFNVGNNDVSWYQQLPGAAPKLLIYDNNK
RPSGIPDRFSGSKSGTSATLDITGLHSDDEADYYCGTWDSSLNTGGVFGTGTKVT
VLGSRGGGGSGGGGSGGGGSLEMAEVQLVQSGAEVKKPGASVKVSCKASGYTF
TSYTISWVRQAPGQGLEWMGWISTYNGLTNYAQNLQGRVTMTTDTFTTTAYME
LRSLRSDDTAVYYCVREGSPDYGDFASFDYWGQGTLVTVSS [SEQ ID NO: 706]

TABLE 134
ET200-108

DNA Sequence
cagtctgtgttgacgcagccgccctcagtgtctgcgcccccgggacagaaggtcaccatctcctgctctggaagcagctccaac
attgggaataattatgtatcctggtaccagcagttcccaggaacagcccccaaactcctcatttatgacaataataagcgaccctca
gggatttctgaccgattctctggctccaagtctggcacgtcagccaccctgggcatcgccggactccagactggggacgaggc
cgattattactgcggaacatgggataccagcctgagtggtttttatgtcttcggaagtgggaccaaggtcaccgtcctaggttctag
aggtggtggtggtagcggcggcggcggctctggtggtggtggatccctcgagatggccgaggtccagctggtacagtctgga
gctgaggtgaagaagcctggggcctcagtgaaggtctcctgcaaggcttctggttacacctttaccagctatactatcagctgggt
acgacaggcccctggacaagggcttgagtggatgggatggatcagcacttacaatggtctcacaaactatgcacagaacctcc
agggcagagtcaccatgactacagacacattcacgaccacagcctacatggagctgaggagcctcagatctgacgacacggc
cgtgtattactgtgtgagagaggggtcccccgactacggtgacttcgcgtcctttgactactggggccagggaaccctggtcacc
gtctcctca [SEQ ID NO: 707]
Amino Acid Sequence
QSVLTQPPSVSAPPGQKVTISCSGSSSNIGNNYVSWYQQFPGTAPKLLIYDNNKRP
SGISDRFSGSKSGTSATLGIAGLQTGDEADYYCGTWDTSLSGFYVFGSGTKVTVL
GSRGGGGSGGGGSGGGGSLEMAEVQLVQSGAEVKKPGASVKVSCKASGYTFTS
YTISWVRQAPGQGLEWMGWISTYNGLTNYAQNLQGRVTMTTDTFTTTAYMEL
RSLRSDDTAVYYCVREGSPDYGDFASFDYWGQGTLVTVSS [SEQ ID NO: 708]

TABLE 135
ET200-109

DNA Sequence
ctgcctgtgctgactcagccaccctcagcgtctgcgacccccgggcagagggtcaccatctcttgttctggaaccacctccaaca
tcggaagtaatactgtacactggtaccagcagctcccagggacggcccccaaactcctcatctataataataatcagcggccctc
aggggtccctgaccgattctctggctccaagtctggcacctcagcctccctggccatcagtgggctccggtccgaggatgaggc
tacatattcctgtgcaacatgggatgacagcctgagtggtgtggtcttcggcggagggaccaagctgaccgtcctaggttctaga
ggtggtggtggtagcggcggcggcggctctggtggtggtggatccctcgagatggccgaggtccagctggtgcagtctgggg
ctgaggtgaagaagcctgggtcctcggtgaaggtctcctgcaaggcttctggaggcaccttcagcagctatgctatcagctgggt
gcgacaggcccctggacaagggcttgagtggatgggagggatcatccctatctttggtacagcaaactacgcacagaagttcc
agggcagagtcacgattaccgcggacgaatccacgagcacagcctacatggagctgagcagcctgagatctgaggacacgg
ccgtgtattactgtgcgagagatcccgcctacggtgactacgagtatgatgcttttgatatctggggccaagggacaatggtcacc
gtctcttca [SEQ ID NO: 709]
Amino Acid Sequence
LPVLTQPPSASATPGQRVTISCSGTTSNIGSNTVHWYQQLPGTAPKLLIYNNNQRP
SGVPDRFSGSKSGTSASLAISGLRSEDEATYSCATWDDSLSGVVFGGGTKLTVLG
SRGGGGSGGGGSGGGGSLEMAEVQLVQSGAEVKKPGSSVKVSCKASGGTFSSY
AISWVRQAPGQGLEWMGGIIPIFGTANYAQKFQGRVTITADESTSTAYMELSSLR
SEDTAVYYCARDPAYGDYEYDAFDIWGQGTMVTVSS [SEQ ID NO: 710]

TABLE 136
ET200-110

DNA Sequence
cagtctgtgttgacgcagccgccctcagcgtctgggacccccgggcagagggtcaccatctcttgttctggaagcagctccaac
atcggaactaatggtgtaaactggttccagcagttcccaggaacggcccccaaactcctcatctatactaatgatcagcggccctc
aggggtccctgaccgattctctggctccaagtctggcacctcagcctccctggccatcagtgggctccagtctgcggatgaggct
gattattactgtgcagtgtgggaccacagcctgaatggtccggtgttcggcggagggaccaagctgaccgtcctaggttctaga
ggtggtggtggtagcggcggcggcggctctggtggtggtggatccctcgagatggcccaggtgcagctggtgcagtctgggg
ctgaggtgaagaagcctgggtcctcggtgaaggtctcctgcaaggcttctggaggcaccttcagcagctatgctatcagctgggt
gcgacaggcccctggacaagggcttgagtggatgggagggatcatccctatctttggtacagcaaactacgcacagaagttcc
agggcagagtcacgattaccgcggacgaatccacgagcacagcctacatggagctgagcagcctgagatctgaggacacgg
ccgtgtattactgtgcgagaggggccggttttgatgcttttgatatctggggccaagggacaatggtcaccgtctcttca [SEQ
ID NO: 711]
Amino Acid Sequence
QSVLTQPPSASGTPGQRVTISCSGSSSNIGTNGVNWFQQFPGTAPKLLIYTNDQRP
SGVPDRFSGSKSGTSASLAISGLQSADEADYYCAVWDHSLNGPVFGGGTKLTVL
GSRGGGGSGGGGSGGGGSLEMAQVQLVQSGAEVKKPGSSVKVSCKASGGTFSS
YAISWVRQAPGQGLEWMGGIIPIFGTANYAQKFQGRVTITADESTSTAYMELSSL
RSEDTAVYYCARGAGFDAFDIWGQGTMVTVSS [SEQ ID NO: 712]

TABLE 137
ET200-111

DNA Sequence
caggctgtgctgactcagccaccctcagcgtctgggacccccgggcagagggtcaccatctcttgttctggaagcagctccaac
atcggaagtaatactgtaaactggtaccagcagctcccaggaacggcccccaaactcctcatctatagtaataatcagcggccct
caggggtccctgaccgattctctggctccaagtctggcacctcagcctccctggccatcagtgggctccagtctgaggatgaga
ctgattattactgtgcagcatgggatgacagcctgaatggttatgtcttcggaactgggaccaaggtcaccgtcctaggttctaga
ggtggtggtggtagcggcggcggcggctctggtggtggtggatccctcgagatggcccaggtgcagctacagcagtggggc
gcaggactgttgaagccttcggagaccctgtccctcacctgcgctgtctatggtgggtccttcagtggttactactggagctggat
ccgccagcccccagggaaggggctggagtggattggggaaatcaatcatagtggaagcaccaactacaacccgtccctcaag
agtcgagtcaccatatcagtagacacgtccaagaaccagttctccctgaagctgagctctgtgaccgccgcggacacggctgtg
tattactgtgcgagagaggggctagatgcttttgatatctggggccaagggacaatggtcaccgtctcttca [SEQ ID NO:
713]
Amino Acid Sequence
QAVLTQPPSASGTPGQRVTISCSGSSSNIGSNTVNWYQQLPGTAPKLLIYSNNQRP
SGVPDRFSGSKSGTSASLAISGLQSEDETDYYCAAWDDSLNGYVFGTGTKVTVL
GSRGGGGSGGGGSGGGGSLEMAQVQLQQWGAGLLKPSETLSLTCAVYGGSFSG
YYWSWIRQPPGKGLEWIGEINHSGSTNYNPSLKSRVTISVDTSKNQFSLKLSSVT
AADTAVYYCAREGLDAFDIWGQGTMVTVSS [SEQ ID NO: 714]

TABLE 138
ET200-112

DNA Sequence
caggctgtgctgactcagccaccctcagcgtctgggacccccgggcagagggtcaccatctcttgttctggaagcagctccaac
atcggaagtaatactgtaaactggtaccagcagctcccaggaacggcccccaaactcctcatgtatagtaatgatcagcggccct
caggggtccctgaccgattctctggctccaagtctggcacctcagcctccctggccatcagtgggctccagtctgaggatgagg
ctgattattattgtgcagcatgggatgacagcctgaatggttatgtcttcgcagctgggacccagctcaccgttttaagttctagagg
tggtggtggtagcggcggcggcggctctggtggtggtggatccctcgagatggcccaggtgcagctacagcagtggggcgc
aggactgttgaagccttcggagaccctgtccctcacctgcgctgtctatggtgggtccttcagtggttactactggagctggatcc
gccagcccccagggaaggggctggagtggattggggaaatcaatcatagtggaagcaccaactacaacccgtccctcaagag
tcgagtcaccatatcagtagacacgtccaagaaccagttctccctgaagctgagctctgtgaccgccgcggacacggctgtgtat
tactgtgcgagagaggggctagatgcttttgatatctggggccaagggacaatggtcaccgtctcttca [SEQ ID NO:
715]
Amino Acid Sequence
QAVLTQPPSASGTPGQRVTISCSGSSSNIGSNTVNWYQQLPGTAPKLLMYSNDQR
PSGVPDRFSGSKSGTSASLAISGLQSEDEADYYCAAWDDSLNGYVFAAGTQLTV
LSSRGGGGSGGGGSGGGGSLEMAQVQLQQWGAGLLKPSETLSLTCAVYGGSFS
GYYWSWIRQPPGKGLEWIGEINHSGSTNYNPSLKSRVTISVDTSKNQFSLKLSSV
TAADTAVYYCAREGLDAFDIWGQGTMVTVSS [SEQ ID NO: 716]

TABLE 139
ET200-113

DNA Sequence
cagtctgtcgtgacgcagccgccctcagtgtctgcggccccaggacagaaggtcaccatctcctgctctggaagcagctccaa
cattgggaataattatgtatcctggtaccagcagctcccaggaacagcccccaaactcctcatttatgacaataataagcgaccct
cagggattcctgaccgattctctggctccaagtctggcacgtcagccaccctgggcatcactggactccagactggggacgag
gccgattattactgcggaacatgggatagcagcctgagtgctgcttatgtcttcggaactgggaccaaggtcaccgtcctaggttc
tagaggtggtggtggtagcggcggcggcggctctggtggtggtggatccctcgagatggcccaggtccagctggtacagtctg
gagctgaggtgaagaagcctggggcctcagtgaaggtctcctgcaaggcttctggttacagctttaccagctatactatcagctg
ggttcgacaggcccctggacaaggccttgagtggatgggatgggtcagcacttacaatggtctcagaaactatgcacagaacct
ccagggcagagtcaccatgactacagacacactcacgaccacagcctacatggagctgaggagcctcagatctgacgacacg
gccgtgtattattgtgtgagagaggggtcccccgactacggtgacttcgcggcctttgactactggggccagggcaccctggtc
accgtctcctca [SEQ ID NO: 717]
Amino Acid Sequence
QSVVTQPPSVSAAPGQKVTISCSGSSSNIGNNYVSWYQQLPGTAPKLLIYDNNKR
PSGIPDRFSGSKSGTSATLGITGLQTGDEADYYCGTWDSSLSAAYVFGTGTKVTV
LGSRGGGGSGGGGSGGGGSLEMAQVQLVQSGAEVKKPGASVKVSCKASGYSFT
SYTISWVRQAPGQGLEWMGWVSTYNGLRNYAQNLQGRVTMTTDTLTTTAYME
LRSLRSDDTAVYYCVREGSPDYGDFAAFDYWGQGTLVTVSS [SEQ ID NO: 718]

TABLE 140
ET200-114

DNA Sequence
caggctgtgctgactcagccaccctcagcgtctgagacccccgggcagagggtcaccatctcttgttctggaagcaggtccaac
atcggaactaatattgtacactggtaccagcagcgcccaggaatggcccccaaactcctcacttatggtagtcggcggccctca
ggggtcccggaccgattctctggctccaagtttggcacctcagcctccctggccatcagtgggctccagtctgaggatgaggct
gattattattgtgcagcatgggatgacagtctgaatggtccggctttcggcggagggaccaagctgaccgtcctaggttctagag
gtggtggtggtagcggcggcggcggctctggtggtggtggatccctcgagatggcccaggtgcagctacagcagtggggcg
caggactgttgaagccttcggagaccctgtccctcacctgcgctgtctatggtgggtccttcagtggttactactggagctggatc
cgccagcccccagggaaggggctggagtggattggggaaatcaatcatagtggaagcaccaactacaacccgtccctcaaga
gtcgagtcaccatatcagtagacacgtccaagaaccagttctccctgaagctgagctctgtgaccgccgcggacacggctgtgt
attactgtgcgagagacggtgggggctactttgactactggggccagggaaccctggtcaccgtctcctca [SEQ ID NO:
719]
Amino Acid Sequence
QAVLTQPPSASETPGQRVTISCSGSRSNIGTNIVHWYQQRPGMAPKLLTYGSRRP
SGVPDRFSGSKFGTSASLAISGLQSEDEADYYCAAWDDSLNGPAFGGGTKLTVL
GSRGGGGSGGGGSGGGGSLEMAQVQLQQWGAGLLKPSETLSLTCAVYGGSFSG
YYWSWIRQPPGKGLEWIGEINHSGSTNYNPSLKSRVTISVDTSKNQFSLKLSSVT
AADTAVYYCARDGGGYFDYWGQGTLVTVSS [SEQ ID NO: 720]

TABLE 141
ET200-115

DNA Sequence
cagtctgtgttgacgcagccgccctcagtgtctggggccccagggcagagggtcaccatctcctgcactgggagcagctccaatatcggggca
cgttatgatgtacactggtaccagcaactcccaggaacagccccccgactcctcatctctgctaactacgatcggccctcaggggtccctgaccg
attctctggctccaagtctggcacctcagcctccctggccatcactgggctccaggctgaggatgaggctgattattactgccagtcctatgacag
cagtgtgagtgcttgggtgttcggcggagggaccaaggtcaccgtcctaggttctagaggtggtggtggtagcggcggcggcggctctggtggt
ggtggatccctcgagatggccgaagtgcagctggtgcagtctggggctgaagtgaaggagcctggggcctcagtgaggatctcctgccaggc
atctggatacaacttcatcagttattatatgcactgggtgcggcaggcccctgggcaaggtcttgagtggatgggcaccatcaacccaggcagtg
gtgagacagactactcacagaagttgcagggcagagtcaccatgaccagggacccgtccacgggtacattcgacatggggctgagcagcctg
acatctggggacacggccgtctattattgtgcgacaggtctcatcagaggagctagcgatgcttttaatatctggggccgggggacaatggtcacc
gtctcttca [SEQ ID NO: 721]
Amino Acid Sequence
QSVLTQPPSVSGAPGQRVTISCTGSSSNIGARYDVHWYQQLPGTAPRLLISANYDRPSGVPD
RFSGSKSGTSASLAITGLQAEDEADYYCQSYDSSVSAWVFGGGTKVTVLGSRGGGGSGGG
GSGGGGSLEMAEVQLVQSGAEVKEPGASVRISCQASGYNFISYYMHWVRQAPGQGLEWM
GTINPGSGETDYSQKLQGRVTMTRDPSTGTFDMGLSSLTSGDTAVYYCATGLIRGASDAFNI
WGRGTMVTVSS [SEQ ID NO: 722]

TABLE 142
ET200-116

DNA Sequence
cagcctgtgctgactcagccaccctcagtgtccgtgtccccaggacagacggccgccatcccctgttctggagataagttgggggataaatttgct
tcctggtatcagcagaagccaggccagtcccctgtgctggtcatctatcaagatactaagcggccctcagggatccctgagcgattctctggctcc
aactctgggaacacagccactctgaccatcagcgggacccaggctatggatgaggctgactattactgtcagacgtgggccagcggcattgtgg
tgttcggcggagggaccaagctgaccgtcctaggttctagaggtggtggtggtagcggcggcggcggctctggtggtggtggatccctcgaga
tggcccaggtacagctgcagcagtcaggtccaggactggtgaagccctcgcagaccctctcactcacctgtgccatctccggggacagtgtctct
agcaacagtgctgcttggaactggatcaggcagtccccatcgagaggccttgagtggctgggaaggacatactacaggtccaagtggtataatg
attatgcagtatctgtgaaaagtcgaataaccatcaacccagacacatccaagaaccagttctccctgcagctgaactctgtgactcccgaggaca
cggctgtgtattactgtgcaagagagcgcagtggctggaagggatttgactactggggccagggaaccctggtcaccgtctcctca [SEQ ID
NO: 723]
Amino Acid Sequence
QPVLTQPPSVSVSPGQTAAIPCSGDKLGDKFASWYQQKPGQSPVLVIYQDTKRPSGIPERFSG
SNSGNTATLTISGTQAMDEADYYCQTWASGIVVFGGGTKLTVLGSRGGGGSGGGGSGGGG
SLEMAQVQLQQSGPGLVKPSQTLSLTCAISGDSVSSNSAAWNWIRQSPSRGLEWLGRTYYR
SKWYNDYAVSVKSRITINPDTSKNQFSLQLNSVTPEDTAVYYCARERSGWKGFDYWGQGT
LVTVSS [SEQ ID NO: 724]

TABLE 143
ET200-117

DNA Sequence
gatgttgtgatgactcagtctccaccctccctgtccgtcacccctggagagccggcctccatcacctgcaggtctagtcagagcctcctggaaaga
aatgcatacaactacttggattggtacctgcagaggccaggacagtctccacagctcctgatctacttgggttctaatcgggccgccggggtccct
gacaggttcagtggcagtggatcaggcagagattttacactgaaaatcagcagagtggagcctgaggatgttggggtttattactgcatgcaagct
ctacaagctccgttcactttcggcggagggaccaaggtggagatcaaacgttctagaggtggtggtggtagcggcggcggcggctctggtggt
ggtggatccctcgagatggccgaagtgcagctggtgcagtctgggggaggcttggtacagcctggggggtccctgagactctcctgtgcagcct
ctggattcacctttagcagctatgccatgagctgggtccgccaggctccagggaaggggctggagtgggtctcagctattagtggtagtggtggt
agcacatactacgcagactccgtgaagggccggttcaccatctccagagacaattccaagaacacgctgtatctgcaaatgaacagcctgagag
ccgaggacacggccgtatattactgtgcgaaatggggcccgtttcaggatgcttttgatatctggggccaagggacaatggtcaccgtctcttca
[SEQ ID NO: 725]
Amino Acid Sequence
DVVMTQSPPSLSVTPGEPASITCRSSQSLLERNAYNYLDWYLQRPGQSPQLLIYLGSNRAAG
VPDRFSGSGSGRDFTLKISRVEPEDVGVYYCMQALQAPFTFGGGTKVEIKRSRGGGGSGGG
GSGGGGSLEMAEVQLVQSGGGLVQPGGSLRLSCAASGFTFSSYAMSWVRQAPGKGLEWV
SAISGSGGSTYYADSVKGRFTISRDNSKNTLYLQMNSLRAEDTAVYYCAKWGPFQDAFDIW
GQGTMVTVSS [SEQ ID NO: 726]

TABLE 144
ET200-118

DNA Sequence
caggctgtgctgactcagcctgcctccgtgtctgggtctcctggacagtcgatcaccatctcctgcactggaaccagcagtgacgttggtggttat
aactatgtctcctggtaccaacagcacccgggcaaagcccccaaactcatgatttatgaggtcagtaatcggccctcaggggtttctaatcgcttc
tctggctccaagtctggcaacacggcctccctgaccatctctgggctccaggctgaggacgaggctgattattactgcagctcatatacaagcagc
agcaccccttatgtcttcggagcagggaccaaggtcaccgtcctaggttctagaggtggtggtggtagcggcggcggcggctctggtggtggtgga
tccctcgagatggccgaggtgcagctggtggagtctgggggaggcttggtacagcctggcaggtccctgagactctcctgtgcagcctctggatt
cacctttgatgattatgccatgcactgggtccggcaagctccagggaagggcctggagtgggtctcaggtattagttggaatagtggtagcatagg
ctatgcggactctgtgaagggccgattcaccatctccagagacaacgccaagaactccctgtatctgcaaatgaacagtctgagagctgaggaca
cggccttgtattactgtgcaaaagccaggtggacagcagtggcatcagaccaccactttgactactggggccagggaacgctggtcaccgtctc
ctca [SEQ ID NO: 727]
Amino Acid Sequence
QAVLTQPASVSGSPGQSITISCTGTSSDVGGYNYVSWYQQHPGKAPKLMIYEVSNRPSGVSN
RFSGSKSGNTASLTISGLQAEDEADYYCSSYTSSSTPYVFGAGTKVTVLGSRGGGGSGGGGS
GGGGSLEMAEVQLVESGGGLVQPGRSLRLSCAASGFTFDDYAMHWVRQAPGKGLEWVSG
ISWNSGSIGYADSVKGRFTISRDNAKNSLYLQMNSLRAEDTALYYCAKARWTAVASDHHF
DYWGQGTLVTVSS [SEQ ID NO: 728]

TABLE 145
ET200-119

DNA Sequence
caggctgtgcttactcagccaccctcagcgtctgggacccccgggcagagggtcaccatctcttgttctggaagcagctccaacatcggaagtaa
tactgtaaactggtaccagcagctcccaggaacggcccccaaactcctcatctatagtaataatcagcggccctcaggggtccctgaccgattctc
tggctccaagtctggcacctcagcctccctggccatcagtgggctccagtctgaggatgaggctgattattactgtgcagcatgggatgacagcct
gaatggttatgtcttcggaactgggaccaagctgaccgtcctaggttctagaggtggtggtggtagcggcggcggcggctctggtggtggtggat
ccctcgagatggccgaggtgcagctggtgcagtctggggctgaggtgaagaagcctgggtcctcggtgaaggtctcctgcaaggcttctggag
gcaccttcagcagctatgctatcagctgggtgcgacaggcccctggacaagggcttgagtggatgggagggatcatccctatctttggtacagca
aactacgcacagaagttccagggcagagtcacgattaccgcggacgaatccacgagcacagcctacatggagctgagcagcctgagatctga
ggacacggccgtgtattactgtgcgagagattgggactacatggacgtctggggcaaagggaccacggtcaccgtctcctca [SEQ ID
NO: 729]
Amino Acid Sequence
QAVLTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYVFGTGTKLTVLGSRGGGGSGGGGS
GGGGSLEMAEVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARDWDYMDVWGKGTT
VTVSS [SEQ ID NO: 730]

TABLE 146
ET200-120

DNA Sequence
tcctatgagctgactcagccaccctcagcgtctgggacccccgggcagagggtcaccatctcttgttctggaagcagctccaacatcggaagtaa
tactgtaaactggtaccagcagctcccaggaacggcccccaaactcctcatctatagtaataatcagcggccctcaggggtccctgaccgattctc
tggctccaagtctggcacctcagcctccctggccatcagtgggctccagtctgaggatgaggctgattattactgtgcagcatgggatgacagcct
gaatggttatgtcttcggaactgggaccaaggtcaccgtcctaggttctagaggtggtggtggtagcggcggcggcggctctggtggtggtggat
ccctcgagatggccgaggtgcagctggtggagtctggagctgaggtgaagaagcctggggcctcagtgaaggtctcctgcaaggcttctggtta
cacctttaccagctatggtatcagctgggtgcgacaggcccctggacaagggcttgagtggatgggatggatcagcgcttacaatggtaacacaa
actatgcacagaagctccagggcagagtcaccatgaccacagacacatccacgagcacagcctacatggagctgaggagcctgagatctgac
gacacggccgtgtattactgtgcgagagacctatctcggggagctaacccgcattactactactactacggtatggacgtctggggccaagggac
cacggtcaccgtctcctca [SEQ ID NO: 731]
Amino Acid Sequence
SYELTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYVFGTGTKVTVLGSRGGGGSGGGGS
GGGGSLEMAEVQLVESGAEVKKPGASVKVSCKASGYTFTSYGISWVRQAPGQGLEWMGW
ISAYNGNTNYAQKLQGRVTMTTDTSTSTAYMELRSLRSDDTAVYYCARDLSRGANPHYYY
YYGMDVWGQGTTVTVSS [SEQ ID NO: 732]

TABLE 147
ET200-121

DNA Sequence
cagtctgtgttgacgcagccgccctcagtgtctggggccccagggcagagggtcaccgtctcctgcactgggagcagatccaacatcggggca
ggatatgatgtacactggtaccagcaacttccaggaacagcccccaaactcctcatctatggaaatagtaatcggcctccaggggtccctgaccga
ttctctgggtctaagtctggcacctcagcctccctggtcatcactgggctccaggctgaggatgccgctgattattactgccagtcctatgacaac
actgtgcgtgaatcaccttatgtcttcggaactgggaccaaggtcaccgtcctaggttctagaggtggtggtggtagcggcggcggcggctctggt
ggtggtggatccctcgagatggccgaggtccagctggtacagtctggggctgaggtgaagaagcctggggcctcagtgaaggtctcctgcaag
gtttccggatacaccctcactgaattatccatgcactgggtgcgacaggctcctggaaaagggcttgagtggatgggaggttttgatcctgaagat
ggtgaaacaatctacgcacagaagttccagggcagagtcaccatgaccgaggacacatctacagacacagcctacatggagctgagcagcctg
agatctgaggacacggccgtgtattactgtgcaacagagagtaatttagtgtcccggcactactactactacggtatggacgtctggggccaagg
gaccacggtcaccgtctcctca [SEQ ID NO: 733]
Amino Acid Sequence
QSVLTQPPSVSGAPGQRVTVSCTGSRSNIGAGYDVHWYQQLPGTAPKLLIYGNSNRPPGVP
DRFSGSKSGTSASLVITGLQAEDAADYYCQSYDNTVRESPYVFGTGTKVTVLGSRGGGGSG
GGGSGGGGSLEMAEVQLVQSGAEVKKPGASVKVSCKVSGYTLTELSMHWVRQAPGKGLE
WMGGFDPEDGETIYAQKFQGRVTMTEDTSTDTAYMELSSLRSEDTAVYYCATESNLVSRH
YYYYGMDVWGQGTTVTVSS [SEQ ID NO: 734]

TABLE 148
ET200-122

DNA Sequence
ctgcctgtgctgactcagccaccctcagcgtctgggacccccgggcagagggtcaccatctcttgttctggaaccagctccaacatcggaagtaa
ttctgtagactggtaccagcagctcccaggaacggcccccaaactcctcatctatagtaataatcagcggccctcaggggtccctgaccgaatctc
tggctccaagtctggcacctcagcctccctggccatcagtgggctccagtctgaggatgaggctgattattactgtgcagcatgggatgacagcct
gaatggttatgtcttcggaactgggaccaaggtcaccgtcctaggttctagaggtggtggtggtagcggcggcggcggctctggtggtggtggat
ccctcgagatggccgaagtgcagctggtgcagtctggggctgaggtgaagaagcctggggcctcagtgaaggtctcctgcaaggcttctggat
acaccttcaccggctactatatgcactgggtgcgacaggcccctggacaagggcttgagtggatgggatggatcaaccctaacagtggtggcac
aaactatgcacagaagtttcagggcagggtcaccatgaccagggacacgtccatcagcacagcctacatggagctgagcaggctgagatctga
cgacacggccgtgtattactgtgcgagagattacggatactatggttcggggagttattcgagcggccccctttactactactacggtatggacgt
ctggggccaagggaccacggtcaccgtctcctca [SEQ ID NO: 735]
Amino Acid Sequence
LPVLTQPPSASGTPGQRVTISCSGTSSNIGSNSVDWYQQLPGTAPKLLIYSNNQRPSGVPDRIS
GSKSGTSASLAISGLQSEDEADYYCAAWDDSLNGYVFGTGTKVTVLGSRGGGGSGGGGSG
GGGSLEMAEVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCARDYGYYGSGSYS
SGPLYYYYGMDVWGQGTTVTVSS [SEQ ID NO: 736]

TABLE 149
ET200-123

DNA Sequence
caggctgtgctgactcagccaccctcagcgtctgggacccccgggcagagggtcaccatctcttgttctggaagcagctccaacatcggaagta
atactgtaaactggtaccagcagctcccaggaacggcccccaaactcctcatgtataataatgatcagcggccctcaggggtccctgaccgattct
ctggctccaagtctggcacctcagcctccctggccatcagtgggctccagtctgaggatgaggctgattattactgtgcagcatgggatgacagc
ctcaatggttatgtcttcggacctgggaccaaggtcaccgtcctaggttctagaggtggtggtggtagcggcggcggcggctctggtggtggtgg
atccctcgagatggcccaggtgcagctggtggagtctggagctgaggtgaagaagcctggggcctcagtgaaggtctcctgcaaggcttctggt
tacacctttaccagctatggtatcagctgggtgcgacaggcccctggacaagggcttgagtggatgggatggatcagcgcttacaatggtaacac
aaactatgcacagaagctccagggcagagtcaccatgaccacagacacatccacgagcacagcctacatggagctgaggagcctgagatctg
acgacacggccgtgtattactgtgcgagagacctatctcggggagctaacccgcattactactactactacggtatggacgtctggggccaaggg
accacggtcaccgtctcctca [SEQ ID NO: 737]
Amino Acid Sequence
QAVLTQPPSASGTPGQRVTISCSGSSSNIGSNTVNWYQQLPGTAPKLLMYNNDQRPSGVPD
RFSGSKSGTSASLAISGLQSEDEADYYCAAWDDSLNGYVFGPGTKVTVLGSRGGGGSGGG
GSGGGGSLEMAQVQLVESGAEVKKPGASVKVSCKASGYTFTSYGISWVRQAPGQGLEWM
GWISAYNGNTNYAQKLQGRVTMTTDTSTSTAYMELRSLRSDDTAVYYCARDLSRGANPH
YYYYYGMDVWGQGTTVTVSS [SEQ ID NO: 738]

TABLE 150
ET200-125

DNA Sequence
aattttatgctgactcagccccacgctgtgtcggagtctccggggaagacggtaaccatctcctgcacccgcagcagtggcagtattgccagcaa
ctatgtgcagtggtaccagcagcgcccgggcagttccccccgcactgtgatttatgaggataatcaaagaccctctggggtccctggtcggttctc
tggctccatcgacagctcctccaactctgcctccctcaccatctctggactgaagactgaggacgaggctgactactactgtcagtcttatgattc
caccagtgtgcttttcggcggagggaccaagctgaccgtcctaggttctagaggtggtggtggtagcggcggcggcggctctggtggtggtggatc
cctcgagatggccgaggtccagctggtgcagtctggggctgaggtgaagaagccagggtcctcggtgaaggtctcctgcaaggcctcgggag
gcaccttcagcagcaattctctcagctgggtgcgacaggcccctggacaagggcttgagtggatgggaaggatcttccctatcctgggtataaca
aactatgcacagaagttccagggcagagtcacgattaccgcggacaaatccacgagcacagcctacatggagctgagcagcctgagatctgag
gacacggccgtctattactgtgcgagaggaaactaccaatggtatgatgcttttgatatctggggccaagggacaatggtcaccgtctcttca
[SEQ ID NO: 739]
Amino Acid Sequence
NFMLTQPHAVSESPGKTVTISCTRSSGSIASNYVQWYQQRPGSSPRTVIYEDNQRPSGVPGR
FSGSIDSSSNSASLTISGLKTEDEADYYCQSYDSTSVLFGGGTKLTVLGSRGGGGSGGGGSG
GGGSLEMAEVQLVQSGAEVKKPGSSVKVSCKASGGTFSSNSLSWVRQAPGQGLEWMGRIF
PILGITNYAQKFQGRVTITADKSTSTAYMELSSLRSEDTAVYYCARGNYQWYDAFDIWGQG
TMVTVSS [SEQ ID NO: 740]

TABLE 151
ET200-005

DNA Sequence
cagcctgtgctgactcagccaccctcagtgtcagtggtcccaggaaagacggccaggattacctgtgggggaaaaaacattggaagtaaaagtg
tgcactggtaccagcagaagccaggccaggcccctgtggtggtcatccattatgatagtgaccggccctcagggatccctgagcgattctctggc
tccaactctgggaacacggccaccctgaccatcagcagggtcgaagccggggatgaggccgactattactgtcaggtgtgggatagtagtagtg
atcatccttatgtcttcggaactgggaccaaggtcaccgtcctaggttctagaggtggtggtggtagcggcggcggcggctctggtggtggtgga
tccctcgagatggcccaggtgcagctggtgcagtctggagctgaggtgaagaagcctggggcctcagtgaaggtctcctgcaaggcttctggtt
acacctttaccaactatggtatcagctgggtgcgacaggcccctggacaagggcttgagtggatgggatggatcagcgcttacaatggtaacaca
aactatgcacataagctccagggcagagtcaccatgaccacagacacatccacgagcacagccaacatggagctgaggagcctgagacctga
cgacactgccgtgtattactgtgcgcgctcttacttcggttctcatgattactggggtcaaggtactctggtgaccgtctcctca [SEQ ID
NO: 741]
Amino Acid Sequence
QPVLTQPPSVSVVPGKTARITCGGKNIGSKSVHWYQQKPGQAPVVVIHYDSDRPSGIPERFS
GSNSGNTATLTISRVEAGDEADYYCQVWDSSSDHPYVFGTGTKVTVLGSRGGGGSGGGGS
GGGGSLEMAQVQLVQSGAEVKKPGASVKVSCKASGYTFTNYGISWVRQAPGQGLEWMG
WISAYNGNTNYAHKLQGRVTMTTDTSTSTANMELRSLRPDDTAVYYCARSYFGSHDYWG
QGTLVTVSS [SEQ ID NO: 742]

TABLE 152
ET200-124

DNA Sequence
tcctatgtgctgactcagccaccctcggtgtcagtggccccaggaaagacggccaggatttcctgtgggggaaacgacattggaagtaaaagtgt
tttctggtatcagcagaggccaggccaggcccctgtgttggtcgtctatgatgatagcgaccggccctcagggctccctgagcgattctctggctt
caactctgggaacacggccaccctgaccatcagcagggtcgaagccggggatgaggccgactattactgtcaagtgtgggatagtagtagtgat
cattatgtcttcggaactgggaccaaggtcaccgtcctaggttctagaggtggtggtggtagcggcggcggcggctctggtggtggtggatccct
cgagatggcccaggtgcagctggtggagtctgggggaggcttggtacagcctggcaggtccctgagactctcctgtgcagcctctggattcacc
tttgatgattatgccatgcactgggtccggcaagctccagggaagggcctggagtgggtctcaggtattagttggaatagtggtagcataggctat
gcggactctgtgaagggccgattcaccatctccagagacaacgccaagaactccctgtatctgcaaatgaacagtctgagagctgaggacacgg
ccttgtattactgtgcaaaagatataacctatggttcggggagttatggtgcttttgatatctggggccaagggacaatggtcaccgtctcttca
[SEQ ID NO: 743]
Amino Acid Sequence
SYVLTQPPSVSVAPGKTARISCGGNDIGSKSVFWYQQRPGQAPVLVVYDDSDRPSGLPERFS
GFNSGNTATLTISRVEAGDEADYYCQVWDSSSDHYVFGTGTKVTVLGSRGGGGSGGGGSG
GGGSLEMAQVQLVESGGGLVQPGRSLRLSCAASGFTFDDYAMHWVRQAPGKGLEWVSGI
SWNSGSIGYADSVKGRFTISRDNAKNSLYLQMNSLRAEDTALYYCAKDITYGSGSYGAFDI
WGQGTMVTVSS [SEQ ID NO: 744]

TABLE 153
ET200-001

DNA Sequence
cagtctgtgttgacgcagccaccctcagcgtctgggacccccgggcagagggtcaccatctcttgttctggaagcagctccaacatcggaagtaa
tactgtaaactggtaccagcagctcccaggaacggcccccaaactcctcatctatagtaataatcagcggccctcaggggtccctgaccgattctc
tggctccaagtctggcacctcagcctccctggccatcagtgggctccagtctgaggatgaggctgattattactgtgcagcatgggatgacagcct
gaatggttatgtcttcggaactgggaccaaggtcaccgtcctaggttctagaggtggtggtggtagcggcggcggcggctctggtggtggtggat
ccctcgagatggcccaggtgcagctacagcagtggggcgcaggactgttgaagccttcggagaccctgtccctcacctgcgctgtgtatggtgg
gtccttcagtggttactactggagctggatccgccagcccccagggaaggggctggagtggattggggaaatcaatcatagtggaagcaccaac
tacaacccgtccctcaagagtcgagtcaccatatcagtagacacgtccaagaaccagttctccctgaagctgagctctgtgaccgccgcggacac
ggccgtgtattactgtgcgcgcgaaggtccgtacgacggtttcgattcttggggtcaaggtactctggtgaccgtctcctcaactagtggccaggc
cggccagcaccatcaccatcaccatggcgcatacccgtacgacgttccggactacgcttct [SEQ ID NO: 745]
Amino Acid Sequence
QSVLTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYVFGTGTKVTVLGSRGGGGSGGGGS
GGGGSLEMAQVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCAREGPYDGFDSWGQGTL
VTVSSTSGQAGQHHHHHHGAYPYDVPDYAS [SEQ ID NO: 746]

TABLE 154
ET200-002

DNA Sequence
aattttatgctgactcagccccactctgtgtcggagtctccggggaagac
ggtaaccatctcctgcacccgcagcagtggcagcattgccagcaactatg
tgcagtggtaccagcagcgcccgggcagtgcccccaccactgtgatctat
gaggataaccaaagaccctctggggtccctgatcggttctctggctccat
cgacagctcctccaactctgcctccctcaccatctctggactgaagactg
aggacgaggctgactactactgtcagtcttatgatagcagcaattctgtg
gtattcggcggagggaccaagctgaccgtcctaggttctagaggtggtgg
tggtagcggcggcggcggctctggtggtggtggatccctcgagatggccc
aggtccagctggtacagtctggcactgaggtgaagaagcctggggcctca
gtgagggtcgcctgcaaggcttctggttacccctttaacaaatatgacat
caactgggtgcgacaggcccctggacaagggcttgagtggatgggaggca
tcatccctatctttcgtacaacaaactacgcacagaagttccagggcaga
gtcacgattaccgcggacgaatccacgagcacagcctacatggagctgag
cagcctgagatctgaggacacggccgtatattactgtgcgcgcgaatggt
tctactgggatatctggggtcaaggtactctggtgaccgtctcctcaact
agtggccaggccggccagcaccatcaccatcaccatggcgcatacccgta
cgacgttccggactacgcttct [SEQ ID NO: 747]
Amino Acid Sequence
NFMLTQPHSVSESPGKTVTISCTRSSGSIASNYVQWYQQRPGSAPTTVIY
EDNQRPSGVPDRFSGSIDSSSNSASLTISGLKTEDEADYYCQSYDSSNSV
VFGGGTKLTVLGSRGGGGSGGGGSGGGGSLEMAQVQLVQSGTEVKKPGAS
VRVACKASGYPFNKYDINWVRQAPGQGLEWMGGIIPIFRTTNYAQKFQGR
VTITADESTSTAYMELSSLRSEDTAVYYCAREWFYWDIWGQGTLVTVSST
SGQAGQHHHHHHGAYPYDVPDYAS [SEQ ID NO: 748]

TABLE 155
ET200-003

DNA Sequence
cagtctgtgttgactcagccaccctcagtgtccgtgtccccaggacagac
agccagcatctcctgctctggaaataaattggggactaagtatgtttact
ggtatcagaagaggccaggccagtcccctgtgttggtcatgtatgaagat
aatcagcggccctcagggatcccggagcggttctctggctccaactctgg
gaacacagccactctgaccatcagagggacccagactgtggatgaggctg
actattactgtcaggcgtgggactccgacactttcgtggtcttcggcgga
gggaccaaggtcaccgtcctaggttctagaggtggtggtggtagcggcgg
cggcggctctggtggtggtggatccctcgagatggccgaggtgcagctgg
tggagaccgggggaggcgtggtccagcctgggaggtccctgagactctcc
tgtgcagcctctggattcaccttcagtagttatggcatgcactgggtccg
ccaggctccaggcaaggggctggagtgggtggcagttatatcacatgatg
gaagtaataaatactacgcagactccgtgaagggccgattcaccatctcc
agagacaattccaaggacacgctgtatctgcaaatgaacagcctgagagg
tgaggacacggccgtatattactgtgcgcgctctaaccagtggtctggtt
acttctctttcgattactggggtcaaggtactctggtgaccgtctcctca
actagtggccaggccggccagcaccatcaccatcaccatggcgcataccc
gtacgacgttccggactacgcttct [SEQ ID NO: 749]
Amino Acid Sequence
QSVLTQPPSVSVSPGQTASISCSGNKLGTKYVYWYQKRPGQSPVLVMYED
NQRPSGIPERFSGSNSGNTATLTIRGTQTVDEADYYCQAWDSDTFVVFGG
GTKVTVLGSRGGGGSGGGGSGGGGSLEMAEVQLVETGGGVVQPGRSLRLS
CAASGFTFSSYGMHWVRQAPGKGLEWVAVISHDGSNKYYADSVKGRFTIS
RDNSKDTLYLQMNSLRGEDTAVYYCARSNQWSGYFSFDYWGQGTLVTVSS
TSGQAGQHHHHHHGAYPYDVPDYAS [SEQ ID NO: 750]

TABLE 156
ET200-006

DNA Sequence
tcctatgtgctgactcagccaccctcagtgtcagtggccccaggaaagac
ggccaggattacctgtgggggaaacaacattggaagtaaaagtgtgcact
ggtaccagcagaagccaggccaggcccctgtggtggtcatccattatgat
agcgaccggccctcagggatccctgagcgattctctggctccaactctgg
gaacacggccaccctgaccatcagcagggtcgaagccggggatgaggccg
actattactgtcaggtgtgggatagtagtagtgatcatccttatgtcttc
ggaactgggaccaaggtcaccgtcctaggttctagaggtggtggtggtag
cggcggcggcggctctggtggtggtggatccctcgagatggccgaggtgc
agctggtgcagtctggagctgaggtgaagaagcctggggcctcagtgaag
gtctcctgcaaggcttctggttacacctttaccacctatggtatcagctg
ggtgcgacaggcccctggacaagggcttgagtggatgggatggatcaaca
cttacaatggtcacacaaactatgcacagaagctccagggcagagccaca
atgaccgcagacacatccacgaacacagcctacatggagctgaggagcct
gagatctgacgacactgccgtgtattactgtgcgcgcgttatctacggtt
ctggtgattactggggtcaaggtactctggtgaccgtctcctcaactagt
ggccaggccggccagcaccatcaccatcaccatggcgcatacccgtacga
cgttccggactacgcttct [SEQ ID NO: 751]
Amino Acid Sequence
SYVLTQPPSVSVAPGKTARITCGGNNIGSKSVHWYQQKPGQAPVVVIHYD
SDRPSGIPERFSGSNSGNTATLTISRVEAGDEADYYCQVWDSSSDHPYVF
GTGTKVTVLGSRGGGGSGGGGSGGGGSLEMAEVQLVQSGAEVKKPGASVK
VSCKASGYTFTTYGISWVRQAPGQGLEWMGWINTYNGHTNYAQKLQGRAT
MTADTSTNTAYMELRSLRSDDTAVYYCARVIYGSGDYWGQGTLVTVSSTS
GQAGQHHHHHHGAYPYDVPDYAS [SEQ ID NO: 752]

TABLE 157
ET200-007

DNA Sequence
tcctatgtgctgactcagccactctcagtgtcagtggccccaggaaagac
ggccaggattacctgtgggggaaacaacattggaagtaaaactgtgcact
ggtaccagcagaagccaggccaggcccctgtgctggtcatctattatgat
agcgaccggccctcagggatccctgagcgattctctggctccaactctgg
gaacacggccaccctgaccatcagcagggtcgaagccggggatgaggccg
actattactgtcaggtgtgggatagtagtagtgatcatcgggtgttcggc
ggagggaccaagctgaccgtcctaggttctagaggtggtggtggtagcgg
cggcggcggctctggtggtggtggatccctcgagatggcccaggtgcagc
tgcaggagtcgggcccaggactggtgaagccttcggagaccctgtccctc
acctgcaatgtctctggttactccatcagcagtggttacttttggggctg
gatccggcagcccccagggaaggggctggagtggattgggagtatctatc
atagtaggagcacctactacaacccgtccctcaagagtcgagtcaccata
tcagtagacacgtccaagaaccagttctccctgaagctgaactctgtgac
cgccgcagacacggccgtgtattactgtgcgcgcggttacggttacttcg
attactggggtcaaggtactctggtgaccgtctcctcaactagtggccag
gccggccagcaccatcaccatcaccatggcgcatacccgtacgacgttcc
ggactacgcttct [SEQ ID NO: 753]
Amino Acid Sequence
SYVLTQPLSVSVAPGKTARITCGGNNIGSKTVHWYQQKPGQAPVLVIYYD
SDRPSGIPERFSGSNSGNTATLTISRVEAGDEADYYCQVWDSSSDHRVFG
GGTKLTVLGSRGGGGSGGGGSGGGGSLEMAQVQLQESGPGLVKPSETLSL
TCNVSGYSISSGYFWGWIRQPPGKGLEWIGSIYHSRSTYYNPSLKSRVTI
SVDTSKNQFSLKLNSVTAADTAVYYCARGYGYFDYWGQGTLVTVSSTSGQ
AGQHHHHHHGAYPYDVPDYAS [SEQ ID NO: 754]

TABLE 158
ET200-008

DNA Sequence
caatctgccctgactcagcctgcctccgtgtctgggtctcctggacagtc
gatcaccatctcctgcactggaaccagcagtgacgttggtggttataact
atgtctcctggtaccaacaacacccaggcaaagcccccaaactcatgatt
tatgatgtcagtaatcggccctcaggggtttctaatcgcttctctggctc
caagtctggcaacacggcctccctgaccatctctgggctccaggctgagg
acgaggctgattattactgcagctcatatacaagcagcagcacttcgaag
gtgttcggcggagggaccaagctgaccgtcctaggttctagaggtggtgg
tggtagcggcggcggcggctctggtggtggtggatccctcgagatggccg
aggtgcagctggtggagtctgggggaggtgtggtacggcctggggggtcc
ctgagactctcctgtgcagcctctggattcacctttggtgattatggcat
gagctgggtccgccaagctccagggaaggggctggagtgggtctctggta
ttaattggaatggtggtagcacaggttatgcagactctgtgaagggccga
ttcaccatctccagagacaacgccaagaactccctgtatctgcaaatgaa
cagtctgagagccgaggacacggccgtatattactgtgcgcgctctaaat
acaacttccatgtttactacgattactggggtcaaggtactctggtgacc
gtctcctcaactagtggccaggccggccagcaccatcaccatcaccatgg
cgcatacccgtacgacgttccggactacgcttct [SEQ ID NO:
755]
Amino Acid Sequence
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTSK
VFGGGTKLTVLGSRGGGGSGGGGSGGGGSLEMAEVQLVESGGGVVRPGGS
LRLSCAASGFTFGDYGMSWVRQAPGKGLEWVSGINWNGGSTGYADSVKGR
FTISRDNAKNSLYLQMNSLRAEDTAVYYCARSKYNFHVYYDYWGQGTLVT
VSSTSGQAGQHHHHHHGAYPYDVPDYAS [SEQ ID NO: 756]

TABLE 159
ET200-009

DNA Sequence
cagtctgtgttgacgcagccaccctcagcgtctgggacccccgggcagac
agtcaccatctcttgttctggaagcaactccaacatcggaagtaattatg
tatactggtaccagcagctcccaggaacggcccccaaactcctcatctat
aggaataatcagcggccctcaggggtccctgaccgattctcaggctccaa
gtctggcacctcagcctccctggccatcagtgggctccgctccgaggatg
aggctgattattactgtgcagcatgggatgacagcctgagtgcttatgtc
ttcggaactgggaccaaggtcaccgtcctaggttctagaggtggtggtgg
tagcggcggcggcggctctggtggtggtggatccctcgagatggcccagg
tgcagctggtgcagtctggagctgaggtgaagaagcctggggcctcagtg
aaggtctcctgcaaggcttctggttacacctttaccagctatggtatcag
ctgggtgcgacaggcccctggacaagggcttgagtggatgggatggatca
gcgcttacaatggtaacacaaactatgcacagaagctccagggcagagtc
accatgaccacagacacatccacgagcacagcctacatggagctgaggag
cctgagatctgacgacactgccgtgtattactgtgcgcgctcttctggta
acatggtttcttggaaagatatgtggggtcaaggtactctggtgaccgtc
tcctcaactagtggccaggccggccagcaccatcaccatcaccatggcgc
atacccgtacgacgttccggactacgcttct [SEQ ID NO: 757]
Amino Acid Sequence
QSVLTQPPSASGTPGQTVTISCSGSNSNIGSNYVYWYQQLPGTAPKLLIY
RNNQRPSGVPDRFSGSKSGTSASLAISGLRSEDEADYYCAAWDDSLSAYV
FGTGTKVTVLGSRGGGGSGGGGSGGGGSLEMAQVQLVQSGAEVKKPGASV
KVSCKASGYTFTSYGISWVRQAPGQGLEWMGWISAYNGNTNYAQKLQGRV
TMTTDTSTSTAYMELRSLRSDDTAVYYCARSSGNMVSWKDMWGQGTLVTV
SSTSGQAGQHHHHHHGAYPYDVPDYAS [SEQ ID NO: 758]

TABLE 160
ET200-010

DNA Sequence
caatctgccctgactcagcctgcctccgtgtctgggtctcctggacagtc
gatcaccatctcctgcactggaaccagcagtgacgttggtggttataact
ctgtctcctggtaccaacaacacccaggcaaagcccccagactcatgatt
tatgatgtcagtaatcggccctcaggggtttctaatcgcttctctggctc
caagtctggcaacacggcctccctgaccatctctgggctccaggctgagg
acgaggctgattattactgcagctcatatacaagcagcagcaccccttta
gtcttcggaactgggaccaaggtcaccgtcctaggttctagaggtggtgg
tggtagcggcggcggcggctctggtggtggtggatccctcgagatggccc
aggtgcagctggtgcagtctggggctgaggtgaagaagcctggggcctca
gtgaaggtctcctgcaaggcttctggttacacctttaccagctatggtat
cagctgggtgcgacaggcccctggacaagggcttgagtggatgggatgga
tcagcgcttacaatggtaacacaaactatgcacagaagctccagggcaga
gtcaccatgaccacagacacatccacgagcacagcctacatggagctgag
gagcctgagatctgacgacacggccgtgtattactgtgcgcgcggtgctg
ttgcttaccatgattggggtcaaggtactctggtgaccgtctcctcaact
agtggccaggccggccagcaccatcaccatcaccatggcgcatacccgta
cgacgttccggactacgcttct [SEQ ID NO: 759]
Amino Acid Sequence
QSALTQPASVSGSPGQSITISCTGTSSDVGGYNSVSWYQQHPGKAPRLMI
YDVSNRPSGVSNRFSGSKSGNTASLTISGLQAEDEADYYCSSYTSSSTPL
VFGTGTKVTVLGSRGGGGSGGGGSGGGGSLEMAQVQLVQSGAEVKKPGAS
VKVSCKASGYTFTSYGISWVRQAPGQGLEWMGWISAYNGNTNYAQKLQGR
VTMTTDTSTSTAYMELRSLRSDDTAVYYCARGAVAYHDWGQGTLVTVSST
SGQAGQHHHHHHGAYPYDVPDYAS [SEQ ID NO: 760]

TABLE 161
ET200-011

DNA Sequence
cagtctgtcgtgacgcagccgccctcagtgtctgcggccccaggacagag
ggtcaccatctcctgctctggaagcagctccaacatttcgatttatgatg
tatcctggtatcagcagctcccaggaacagcccccaaactcctcatttat
ggcaataataagcgaccctcggggattgctgaccgattctctggctccac
gtctggcacgtcagccaccctgggcatcaccggactccagactggggacg
aggccgattattactgcggaacatgggatgacagtctgagtgggggggtg
ttcggcggagggaccaagctgaccgtcctaggttctagaggtggtggtgg
tagcggcggcggcggctctggtggtggtggatccctcgagatggcccaga
tgcagctggtgcaatctggggctgaggtgaagaagcctgggtcctcggtg
aaggtctcctgcgaggcttctggaggcaccctcagcagctatgctatcaa
ctgggtgcgacaggcccctggacaagggcttgagtggatgggagggatca
tccctatgtttggtacagcacactacgcacagaagttccagggcagagtc
acgattaccgcggacgaatccacgaaaacagcctacatggagctgagcag
cctgagatctgaggacactgccgtgtattactgtgcgcgcggtgttcatt
acgcttctttcgatcattggggtcaaggtactctggtgaccgtctcctca
actagtggccaggccggccagcaccatcaccatcaccatggcgcataccc
gtacgacgttccggactacgcttct [SEQ ID NO: 761]
Amino Acid Sequence
QSVVTQPPSVSAAPGQRVTISCSGSSSNISIYDVSWYQQLPGTAPKLLIY
GNNKRPSGIADRFSGSTSGTSATLGITGLQTGDEADYYCGTWDDSLSGGV
FGGGTKLTVLGSRGGGGSGGGGSGGGGSLEMAQMQLVQSGAEVKKPGSSV
KVSCEASGGTLSSYAINWVRQAPGQGLEWMGGIIPMFGTAHYAQKFQGRV
TITADESTKTAYMELSSLRSEDTAVYYCARGVHYASFDHWGQGTLVTVSS
TSGQAGQHHHHHHGAYPYDVPDYAS [SEQ ID NO: 762]

TABLE 162
ET200-012

DNA Sequence
cagtctgtgttgacgcagccgccctcagtgtctgcggccgcaggacagaa
ggtcaccatctcctgctctggaagcgactccaacattgggaataattatg
tgtcctggtatcaacacctcccagggacagcccccaaactcctcatttat
gacgttaaaaatcgaccctcagggattcctgaccggttctccggctccaa
gtctggctcgtcagccaccctaggcatcgccggactccagcctggggacg
aggccgattattactgcggaacatgggacagtcggctggatgcctatgtc
ttcggaactgggaccaaggtcaccgtcctaggttctagaggtggtggtgg
tagcggcggcggcggctctggtggtggtggatccctcgagatggcccaga
tgcagctggtgcaatctggagctgaggtgaagaagcctggggcctcagtg
aaggtctcctgcaagacttctggtttcccctttaatatctttggaatcac
ctgggtgcgacaggcccctggacaagggcttgagtggatgggatggatca
gcggttacaacggtaacacagactacccacagaagttccagggcagagtc
accatgtccacagacacatccacgagtacagcctacatggagctgaggaa
cctgaaatctgacgacacggccgtgtattactgtgcgcgcggtgcttacg
gtggtatggatacttggggtcaaggtactctggtgaccgtctcctcaact
agtggccaggccggccagcaccatcaccatcaccatggcgcatacccgta
cgacgttccggactacgcttct [SEQ ID NO: 763]
Amino Acid Sequence
QSVLTQPPSVSAAAGQKVTISCSGSDSNIGNNYVSWYQHLPGTAPKLLIY
DVKNRPSGIPDRFSGSKSGSSATLGIAGLQPGDEADYYCGTWDSRLDAYV
FGTGTKVTVLGSRGGGGSGGGGSGGGGSLEMAQMQLVQSGAEVKKPGASV
KVSCKTSGFPFNIFGITWVRQAPGQGLEWMGWISGYNGNTDYPQKFQGRV
TMSTDTSTSTAYMELRNLKSDDTAVYYCARGAYGGMDTWGQGTLVTVSST
SGQAGQHHHHHHGAYPYDVPDYAS [SEQ ID NO: 764]

TABLE 163
ET200-013

DNA Sequence
cagtctgtcgtgacgcagccgccctcagtgtctggggccccagggcagag
ggtcaccatctcctgcactgggagcacctccaacatcggggcaggttatg
atgtacactggtatcagcagcttccaggaacagcccccaaactcctcatc
tatactaacaactttcggccctcaggggtccctgaccgattctctgcctc
caagtctggcacttcagcttccctggccatcactggtctccaggctgagg
atgaggctgattattactgcggaacatgggatagcagcctgagtgccgtt
gtgttcggcggagggaccaagctgaccgtcctaggttctagaggtggtgg
tggtagcggcggcggcggctctggtggtggtggatccctcgagatggccg
aggtgcagctggtggagtctggaactgaggtgaagaagcctggggcctca
gtgaaagtctcctgcaaggcttctggttacatgtttaccagttatggtct
caactgggtgcgacaggcccctggacaagggcttgagtggatgggatgga
tcagcgctaacaatggtaagacaaattatgctaagaaattccaggacaga
gtcaccatgaccagagacacttccacgagcacaggctacatggaactgag
gagcctgagatctgacgacacggccgtatattactgtgcgcgccatatcg
gtggttcttacttcgatcgttggggtcaaggtactctggtgaccgtctcc
tcaactagtggccaggccggccagcaccatcaccatcaccatggcgcata
cccgtacgacgttccggactacgcttct [SEQ ID NO: 765]
Amino Acid Sequence
QSVVTQPPSVSGAPGQRVTISCTGSTSNIGAGYDVHWYQQLPGTAPKLLI
YTNNFRPSGVPDRFSASKSGTSASLAITGLQAEDEADYYCGTWDSSLSAV
VFGGGTKLTVLGSRGGGGSGGGGSGGGGSLEMAEVQLVESGTEVKKPGAS
VKVSCKASGYMFTSYGLNWVRQAPGQGLEWMGWISANNGKTNYAKKFQDR
VTMTRDTSTSTGYMELRSLRSDDTAVYYCARHIGGSYFDRWGQGTLVTVS
STSGQAGQHHHHHHGAYPYDVPDYAS [SEQ ID NO: 766]

TABLE 164
ET200-014

DNA Sequence
tcctatgtgctgactcagccaccctcagtgtcagtggccccaggaaagac
ggccaggattacctgtgggggaaacaacattggaagtaaaagtgtgcact
ggtaccagcagaagccaggccaggcccctgtgctggtcatctattatgat
agcgaccggccctcagggatccctgagcgattctctggctccaactctgg
gaacacggccaccctgaccatcagcagggtcgaagccggggatgaggccg
actattactgtcaggtgtgggatagtagtagtgatcattatgtcttcgga
actgggaccaaggtcaccgtcctaggttctagaggtggtggtggtagcgg
cggcggcggctctggtggtggtggatccctcgagatggccgaggtgcagc
tggtggagactgggggaggcttggtacagcctggggggtccctgagactc
tcctgtgcagcctctggattcacctttagcagctatgccatgagctgggt
ccgccaggctccagggaaggggctggagtgggtctcagctattagtggta
gtgatggtagcacatactacgcagactccgtgaagggccggttcaccatc
tccagagacaattccaagaacacgctgtatctgcaaatgaacagcctgag
agacgaggacacggccgtatattactgtgcgcgctctcatgaagctaacc
tggttggtgattggtggggtcaaggtactctggtgaccgtctcctcaact
agtggccaggccggccagcaccatcaccatcaccatggcgcatacccgta
cgacgttccggactacgcttct [SEQ ID NO: 767]
Amino Acid Sequence
SYVLTQPPSVSVAPGKTARITCGGNNIGSKSVHWYQQKPGQAPVLVIYYD
SDRPSGIPERFSGSNSGNTATLTISRVEAGDEADYYCQVWDSSSDHYVFG
TGTKVTVLGSRGGGGSGGGGSGGGGSLEMAEVQLVETGGGLVQPGGSLRL
SCAASGFTFSSYAMSWVRQAPGKGLEWVSAISGSDGSTYYADSVKGRFTI
SRDNSKNTLYLQMNSLRDEDTAVYYCARSHEANLVGDWWGQGTLVTVSST
SGQAGQHHHHHHGAYPYDVPDYAS [SEQ ID NO: 768]

TABLE 165
ET200-015

DNA Sequence
cagtctgtggtgactcagccaccctcagtgtcagtggccccaggaaagac
ggccaggattacctgtgggggaaacaacattggaagtaaaagtgtgcact
ggtaccagcagaagccaggccaggcccctgtgctggtcatctattatgat
agcgaccggccctcagggatccctgagcgattctctggctccaactctgg
gaacacggccaccctgaccatcagcagggtcgaagccggggatgaggccg
actattactgtcaggtgtgggatagtagtagtgatgtggtattcggcgga
gggaccaagctgaccgtcctaggttctagaggtggtggtggtagcggcgg
cggcggctctggtggtggtggatccctcgagatggccgaggtccagctgg
tacagtctggagctgaggtgaagaagcctggggcctcagtgaaggtctcc
tgcaaggcttctggttacacctttaccagctacggtatcagctgggtgcg
acaggcccctggacaagggcttgagtggatgggatggatcagcgcttaca
atggtaacacaaactatgcacagaagctccagggcagagtcaccatgacc
acagacacatccacgagcacagcctacatggagctgaggagcctgagatc
tgacgacacggccgtgtattactgtgcgcgctggggtggtttcggtgctg
ttgatcattggggtcaaggtactctggtgaccgtctcctcaactagtggc
caggccggccagcaccatcaccatcaccatggcgcatacccgtacgacgt
tccggactacgcttct [SEQ ID NO: 769]
Amino Acid Sequence
QSVVTQPPSVSVAPGKTARITCGGNNIGSKSVHWYQQKPGQAPVLVIYYD
SDRPSGIPERFSGSNSGNTATLTISRVEAGDEADYYCQVWDSSSDVVFGG
GTKLTVLGSRGGGGSGGGGSGGGGSLEMAEVQLVQSGAEVKKPGASVKVS
CKASGYTFTSYGISWVRQAPGQGLEWMGWISAYNGNTNYAQKLQGRVTMT
TDTSTSTAYMELRSLRSDDTAVYYCARWGGFGAVDHWGQGTLVTVSSTSG
QAGQHHHHHHGAYPYDVPDYAS [SEQ ID NO: 770]

TABLE 166
ET200-016

DNA Sequence
tcttctgagctgactcaggaccctgctgtgtctgtggccttgggacagac
agtcaagatcacgtgccaaggagacagcctcacagactaccatgcaacct
ggtaccagcagaagccaggacaggcccctgtcgctgtcatctatgctaca
aacaaccggcccactgggatcccagaccgattctctggttccagttccgg
aaacacagcttctttgaccatcactggggctcaggcggaagatgaggctg
actattactgtaattcccgggacagcggcacggacgaagtgttattcggc
ggagggaccaagctgaccgtcctaggttctagaggtggtggtggtagcgg
cggcggcggctctggtggtggtggatccctcgagatggccgaggtgcagc
tggtggagactgggggaggcctggtcaagcctggggggtccctgagactc
tcctgtgcagcctctggattcaccttcagtagctatagcatgaactgggt
ccgccaggctccagggaaggggctggagtgggtctcatccattagtagta
gtagtagttacatatactacgcagactcagtgaagggccgattcaccatc
tccagagacaacgccaagaactcactgtatctgcaaatgaacagcctgag
agccgaggacacggccgtgtattactgtgcgcgcggtcagggttacgatt
actggggtcaaggtactctggtgaccgtctcctcaactagtggccaggcc
ggccagcaccatcaccatcaccatggcgcatacccgtacgacgttccgga
ctacgcttct [SEQ ID NO: 771]
Amino Acid Sequence
SSELTQDPAVSVALGQTVKITCQGDSLTDYHATWYQQKPGQAPVAVIYAT
NNRPTGIPDRFSGSSSGNTASLTITGAQAEDEADYYCNSRDSGTDEVLFG
GGTKLTVLGSRGGGGSGGGGSGGGGSLEMAEVQLVETGGGLVKPGGSLRL
SCAASGFTFSSYSMNWVRQAPGKGLEWVSSISSSSSYIYYADSVKGRFTI
SRDNAKNSLYLQMNSLRAEDTAVYYCARGQGYDYWGQGTLVTVSSTSGQA
GQHHHHHHGAYPYDVPDYAS [SEQ ID NO: 772]

TABLE 167
ET200-017

DNA Sequence
Tcctatgtgctgactcagccaccctcggtgtcagtggccccaggaaagac
ggccaggattacctgtgggggaaacaacattggaagtaaaagtgtgcact
ggtaccagcagaagccaggccaggcccctgtgctggtcgtctatgatgat
agcgaccggccctcagggatccctgagcgattctctggctccaactctgg
gaacacggccaccctgagcatcagcagggtcgaagccggggatgaggccg
actattactgtcaggtgtgggatagtagtagtgatcatactgtcttcgga
actgggaccaaggtcaccgtcctaggttctagaggtggtggtggtagcgg
cggcggcggctctggtggtggtggatccctcgagatggcccaggtgcagc
tacagcagtggggcgcaggactgttgaagccttcggagaccctgtccctc
acctgcgctgtctatggtgggtccttcagtggttactactggagctggat
ccgccagcccccagggaaggggctggagtggattggggaaatcaatcata
agtggaagcaccaactacaacccgtccctcaaggtcgagtcaccatatca
gtagacacgtccaagaaccagttctccctgaagctgagctctgtgaccgc
cgcggacacggccgtgtattactgtgcgcgctactacccgggtatggata
tgtggggtcaaggtactctggtgaccgtctcctcaactagtggccaggcc
ggccagcaccatcaccatcaccatggcgcatacccgtacgacgttccgga
ctacgcttct [SEQ ID NO: 773]
Amino Acid Sequence
SYVLTQPPSVSVAPGKTARITCGGNNIGSKSVHWYQQKPGQAPVLVVYDD
SDRPSGIPERFSGSNSGNTATLSISRVEAGDEADYYCQVWDSSSDHTVFG
TGTKVTVLGSRGGGGSGGGGSGGGGSLEMAQVQLQQWGAGLLKPSETLSL
TCAVYGGSFSGYYWSWIRQPPGKGLEWIGEINHSGSTNYNPSLKSRVTIS
VDTSKNQFSLKLSSVTAADTAVYYCARYYPGMDMWGQGTLVTVSSTSGQA
GQHHHHHHGAYPYDVPDYAS [SEQ ID NO: 774]

TABLE 168
ET200-018

DNA Sequence
Caggctgtgctgactcagccgccctcaacgtctgggacccccgggcagag
ggtcaccatctcttgttctggaagcagctccaacatcgggagaaatggtg
taaactggtaccagcagctcccaggagcggcccccaaagtcctcatctat
aatgataatcagcgaccctcaggggtccctgaccgagtctctggctccca
gtctggctcctcaggcaccctggccatcgatgggcttcggtctgaggatg
aggctgattattactgtgcggcatgggatgacagcctgcatggtgtggta
ttcggcggagggaccaagctgaccgtcctaggttctagaggtggtggtgg
tagcggcggcggcggctctggtggtggtggatccctcgagatggcccagg
tccagctggtacagtctggggctgaggtgaagaagcctggggcctcagtg
aaggtctcctgcaaggtttccggatacaccctcaatgaattatccatgca
ctgggtgcgacaggctcctggaaaagggcttgagtggatgggaggttttg
atcctgaagatggtgaaacaatctacgcacagaagttccagggcagagtc
accatgaccgaggacacatctacagacacagcctacatggagctgagcag
cctgagatctgaggacactgccgtgtattactgtgcgcgcggtggttacg
gtgattcttggggtcaaggtactctggtgaccgtctcctcaactagtggc
caggccggccagcaccatcaccatcaccatggcgcatacccgtacgacgt
tccggactacgcttct [SEQ ID NO: 775]
Amino Acid Sequence
QAVLTQPPSTSGTPGQRVTISCSGSSSNIGRNGVNWYQQLPGAAPKVLIY
NDNQRPSGVPDRVSGSQSGSSGTLAIDGLRSEDEADYYCAAWDDSLHGVV
FGGGTKLTVLGSRGGGGSGGGGSGGGGSLEMAQVQLVQSGAEVKKPGASV
KVSCKVSGYTLNELSMHWVRQAPGKGLEWMGGFDPEDGETIYAQKFQGRV
TMTEDTSTDTAYMELSSLRSEDTAVYYCARGGYGDSWGQGTLVTVSSTSG
QAGQHHHHHHGAYPYDVPDYAS [SEQ ID NO: 776]

TABLE 169
ET200-019

DNA Sequence
Aattttatgctgactcagccccactctgtgtcggagtctccggggaagac
ggtaaccatctcctgcacccgcagcagtggcagcattgccagcaactatg
tgcagtggtaccagcagcgcccgggcagtgcccccaccactgtgatctat
gaggataaccaaagaccctctggggtccctgatcggttctctggctccat
cgacagctcctccaactctgcctccctcaccatctctggactgaagactg
aggacgaggctgactactactgtcagtcttatgatagcagcaattcttgg
gtgttcggcggagggaccaagctgaccgtcctaggttctagaggtggtgg
tggtagcggcggcggcggctctggtggtggtggatccctcgagatggccc
aggtgcagctggtgcaatctggggctgaggtgaagaggcctgggtcctcg
gtgaaggtctcctgcacggcttctggaggcaccttcagcagcgatgctat
cagctgggtgcgacaggcccctggacaagggcttgagtggatgggaggaa
tcatccctatgtttggtacagcaaactacgcacagaagttccagggcaga
gtcacgattaccgcggacgaatccacgagcacagcctacatggagctgag
cagcctgagatctgaggacacggccgtgtattactgtgcgcgcgaaggtt
actactacccgtctgcttacctgggttctgttctgaacgacatctcttct
gtttacgatgaatggggtcaaggtactctggtgaccgtctcctcaactag
tggccaggccggccagcaccatcaccatcaccatggcgcata
cccgtacgacgttccggactacgcttct [SEQ ID NO: 777]
Amino Acid Sequence
NFMLTQPHSVSESPGKTVTISCTRSSGSIASNYVQWYQQRPGSAPTTVIY
EDNQRPSGVPDRFSGSIDSSSNSASLTISGLKTEDEADYYCQSYDSSNSW
VFGGGTKLTVLGSRGGGGSGGGGSGGGGSLEMAQVQLVQSGAEVKRPGSS
VKVSCTASGGTFSSDAISWVRQAPGQGLEWMGGIIPMFGTANYAQKFQGR
VTITADESTSTAYMELSSLRSEDTAVYYCAREGYYYPSAYLGSVLNDISS
VYDEWGQGTLVTVSSTSGQAGQHHHHHHGAYPYDVPDYAS
[SEQ ID NO: 778]

TABLE 170
ET200-020

DNA Sequence
Cagtctgtcgtgacgcagccgccctcagtgtctgcggccccaggacagaa
ggtcaccatctcctgctctggaagcacctccaacattggaaataatgatg
tatcctggtaccagcagctcccaggaacagcccccaaactcctcatttat
gacaataataagcgaccctcagggattcctgaccgattctctggctccaa
gtctggcacgtcagccaccctgggcatcaccggactccagactggggacg
aggccgattattactgcggaacatgggatagcagcgtgagtgcttcttgg
gtcttcggcagagggaccaagctgaccgtcctaggttctagaggtggtgg
tggtagcggcggcggcggctctggtggtggtggatccctcgagatggccc
aggtgcagctggtgcagtctggagctgaggtgaagaagcctggggcctca
gtgaaggtctcctgcaaggcttctggttacacctttaccagctatggtat
cagctgggtgcgacaggcccctggacaagggcttgagtggatgggatgga
tcagcgcttacaatggtaacacaaactatccacagaagctccagggcaga
gtcaccatgaccacagacccatccacgagcacagcctacatggagctgag
gagcctgagatctgacgacacggccgtgtattactgtgcgcgctctatga
cttctttcgattactggggtcaaggtactctggtgaccgtctcctcaact
agtggccaggccggccagcaccatcaccatcaccatggcgcatacccgta
cgacgttccggactacgcttct [SEQ ID NO: 779]
Amino Acid Sequence
QSVVTQPPSVSAAPGQKVTISCSGSTSNIGNNDVSWYQQLPGTAPKLLIY
DNNKRPSGIPDRFSGSKSGTSATLGITGLQTGDEADYYCGTWDSSVSASW
VFGRGTKLTVLGSRGGGGSGGGGSGGGGSLEMAQVQLVQSGAEVKKPGAS
VKVSCKASGYTFTSYGISWVRQAPGQGLEWMGWISAYNGNTNYPQKLQGR
VTMTTDPSTSTAYMELRSLRSDDTAVYYCARSMTSFDYWGQGTLVTVSST
SGQAGQHHHHHHGAYPYDVPDYAS [SEQ ID NO: 780]

TABLE 171
ET200-021

DNA Sequence
Cagtctgtgttgacgcagccgccctcagtgtctgcggccccaggacagaa
ggtcaccatctcctgctctggaagcaactccaacattgggaataattatg
tatcctggtatcagcaactcccagggacagcccccaaactcctcatttat
gacaataataagcgaccctcagggattcctgaccgattctctggctccag
gtctggcacgtcagccaccctgggcatcaccggactccagactggggacg
aggccgattattactgcggaacatggaataccactgtgactcctggctat
gtcttcggaactgggaccaaggtcaccgtcctaggttctagaggtggtgg
tggtagcggcggcggcggctctggtggtggtggatccctcgagatggccg
aagtgcagctggtgcagtctggagctgaggtgaagaagcctggggcctca
gtgaaggtctcctgcaaggcttctggttacacctttaccagctatggtat
cagctgggtgcgacaggcccctggacaagggcttgagtggatgggatgga
tcagcgcttacaatggtaacacaaactatgcacagaagctccagggcaga
gtcaccatgaccacagacacatccacgagcacagcctacatggagctgag
gagcctgagatctgacgacaccgccatgtattactgtgcgcgctctgttt
acgacctggatacttggggtcaaggtactctggtgaccgtctcctcaact
agtggccaggccggccagcaccatcaccatcaccatggcgcatacccgta
cgacgttccggactacgcttct [SEQ ID NO: 781]
Amino Acid Sequence
QSVLTQPPSVSAAPGQKVTISCSGSNSNIGNNYVSWYQQLPGTAPKLLIY
DNNKRPSGIPDRFSGSRSGTSATLGITGLQTGDEADYYCGTWNTTVTPGY
VFGTGTKVTVLGSRGGGGSGGGGSGGGGSLEMAEVQLVQSGAEVKKPGAS
VKVSCKASGYTFTSYGISWVRQAPGQGLEWMGWISAYNGNTNYAQKLQGR
VTMTTDTSTSTAYMELRSLRSDDTAMYYCARSVYDLDTWGQGTLVTVSST
SGQAGQHHHHHHGAYPYDVPDYAS [SEQ ID NO: 782]

TABLE 172
ET200-022

DNA Sequence
cagtctgtcgtgacgcagccgccctcagtgtctgcggccccaggacagaa
ggtcaccatctcctgctctggaagcagctccaacattgggaataattatg
tatcctggtaccagcagctcccaggaacagcccccaaactcctcatttat
gacaataataagcgaccctcagggattcctgaccgattctctggctccaa
gtctggcacgtcagccaccctgggcatcaccggactccagactggggacg
aggccgattattactgcggaacatgggatagcagcctgggggccccttat
gtcttcggaactgggaccaaggtcaccgtcctaggttctagaggtggtgg
tggtagcggcggcggcggctctggtggtggtggatccctcgagatggccg
aggtgcagctggtgcagtcttggggaggctcggaacagcctggcaggtcc
ctgagactctcctgtgcagcctctggattcacctttgatgattatgccat
gcactgggtccggcaagctccagggaagggcctggagtgggtctcaggta
ttagttggaatagcggtagcataggctatgcggactctgtgaagggccga
ttcaccatctccagagacaacgccaagaattccctgtatctgcaaatgaa
cagtctgagagctgaggacaccgccatgtattactgtgcgcgctaccgtc
aggttggttctgcttacgattcttggggtcaaggtactctggtgaccgtc
tcctcaactagtggccaggccggccagcaccatcaccatcaccatggcgc
atacccgtacgacgttccggactacgcttct [SEQ ID NO: 783]
Amino Acid Sequence
QSVVTQPPSVSAAPGQKVTISCSGSSSNIGNNYVSWYQQLPGTAPKLLIY
DNNKRPSGIPDRFSGSKSGTSATLGITGLQTGDEADYYCGTWDSSLGAPY
VFGTGTKVTVLGSRGGGGSGGGGSGGGGSLEMAEVQLVQSWGGSEQPGRS
LRLSCAASGFTFDDYAMHWVRQAPGKGLEWVSGISWNSGSIGYADSVKGR
FTISRDNAKNSLYLQMNSLRAEDTAMYYCARYRQVGSAYDSWGQGTLVTV
SSTSGQAGQHHHHHHGAYPYDVPDYAS [SEQ ID NO: 784]

TABLE 173
ET200-023

DNA Sequence
ctgcctgtgctgactcagccaccctcggtgtcagtggccccaggaaagac
ggccaggattacctgtgggggaaacaacattggaagtaaaagtgtgcact
ggtatcagcagaagccaggccaggcccctgtgctggtcgtctatgctgat
agcgaccggccctcagggatccctgagcgattctctggctccaactctgg
gaacacggccaccctgaccatcagcagggtcgaagccggggatgaggccg
actattactgtcaggtgtgggatagtagtagttatcataattatgtcttc
ggaactgggaccaaggtcaccgtcctaggttctagaggtggtggtggtag
cggcggcggcggctctggtggtggtggatccctcgagatggccgaggtgc
agctggtgcagtctggagctgaggtgaagaagcctggggcctcagtgaag
gtctcctgcaaggcttctggttacacctttaccagctatggtatcagctg
ggtgcgacaggcccctggacaagggcttgagtggatgggatggatcagcg
cttacaatggtaacacaaactatgcacagaagctccagggcagagtcacc
atgaccacagacacatccacgagcacagcctacatggagctgagcagcct
gagatctgaggacaccgccatgtattactgtgcgcgctactggggtttcg
gtgtttctgatcgttggggtcaaggtactctggtgaccgtctcctcaact
agtggccaggccggccagcaccatcaccatcaccatggcgcatacccgta
cgacgttccggactacgcttct [SEQ ID NO: 785]
Amino Acid Sequence
LPVLTQPPSVSVAPGKTARITCGGNNIGSKSVHWYQQKPGQAPVLVVYAD
SDRPSGIPERFSGSNSGNTATLTISRVEAGDEADYYCQVWDSSSYHNYVF
GTGTKVTVLGSRGGGGSGGGGSGGGGSLEMAEVQLVQSGAEVKKPGASVK
VSCKASGYTFTSYGISWVRQAPGQGLEWMGWISAYNGNTNYAQKLQGRVT
TMTDTSTSTAYMELSSLRSEDTAMYYCARYWGFGVSDRWGQGTLVTVSST
SGQAGQHHHHHHGAYPYDVPDYAS [SEQ ID NO: 786]

TABLE 174
ET200-024

DNA Sequence
aattttatgctgactcagccccactctgtgtcggagtctccggggaagac
ggtaaccatctcctgcaccggcagcagtggcagcattgccagcaactatg
tgcagtggtaccagcagcgcccgggcagtgcccccaccactgtgatctat
gaggataaccaaagaccctctggggtccccgatcggttctctggctccat
cgacagctcctccaactctgcctccctcaccatctctggactgaagactg
aggacgaggctgactactactgtcagtcttatgacagcagcaatctttgg
gtgttcggcggagggaccaagctgaccgtcctaggttctagaggtggtgg
tggtagcggcggcggcggctctggtggtggtggatccctcgagatggccc
agatgcagctggtgcagtctggggctgaggtgaagaagcctgggtcctcg
gtgaaggtctcctgcaaggcttctggaggcaccttcagcagctatgctat
cagctgggtgcgacaggcccctggacaagggcttgagtggatgggaggga
tcatccctatctttggtacagcaaactacgcacagaagttccagggcaga
gtcacgattaccgcggacgaatccacgagcacagcctacatggagctgag
cagcctgagatctgaggacactgccgtgtattactgtgcgcgctacaact
actactactacgattcttggggtcaaggtactctggtgaccgtctcctca
actagtggccaggccggccagcaccatcaccatcaccatggcgcataccc
gtacgacgttccggactacgcttct [SEQ ID NO: 787]
Amino Acid Sequence
NFMLTQPHSVSESPGKTVTISCTGSSGSIASNYVQWYQQRPGSAPTTVIY
EDNQRPSGVPDRFSGSIDSSSNSASLTISGLKTEDEADYYCQSYDSSNLW
VFGGGTKLTVLGSRGGGGSGGGGSGGGGSLEMAQMQLVQSGAEVKKPGSS
VKVSCKASGGTFSSYAISWVRQAPGQGLEWMGGIIPIFGTANYAQKFQGR
VTITADESTSTAYMELSSLRSEDTAVYYCARYNYYYYDSWGQGTLVTVSS
TSGQAGQHHHHHHGAYPYDVPDYAS [SEQ ID NO: 788]

TABLE 175
ET200-025

DNA Sequence
gacatccagatgacccagtctccatcctccctgtctgcatctgtaggaga
cagagtcaccatcacttgccgggcaagtcagagcattagcagctatttaa
attggtatcagcagaaaccagggaaagcccctaagctcctgatctatgct
gcatccagtttgcaaagtggggtcccatcaaggttcagtggcagtggatc
tgggacagatttcactctcaccatcagcagtctgcaacctgaagattttg
caacttactactgtcaacagagttacagtaccccattcactttcggccct
gggaccaaagtggatatcaaacgttctagaggtggtggtggtagcggcgg
cggcggctctggtggtggtggatccctcgagatggccgaggtgcagctgg
tgcagtctggggctgaggtgaagaagcctgggtcctcggtgaaggtctcc
tgcaaggcttctggaggcaccttcagcagctatgctatcagctgggtgcg
acaggcccctggacaagggcttgagtggatgggagggatcatccctatct
ttggtacagcaaactacgcacagaagttccagggcagagtcacgattacc
gcggacgaatccacgagcacagcctacatggagctgagcagcctgagatc
tgaggacaccgccatgtattactgtgcgcgctactggggttacgactctt
acgatgaatggggtcaaggtactctggtgaccgtctcctcaactagtggc
caggccggccagcaccatcaccatcaccatggcgcatacccgtacgacgt
tccggactacgcttct [SEQ ID NO: 789]
Amino Acid Sequence
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPFTFGP
GTKVDIKRSRGGGGSGGGGSGGGGSLEMAEVQLVQSGAEVKKPGSSVKVS
CKASGGTFSSYAISWVRQAPGQGLEWMGGIIPIFGTANYAQKFQGRVTIT
ADESTSTAYMELSSLRSEDTAMYYCARYWGYDSYDEWGQGTLVTVSSTSG
QAGQHHHHHHGAYPYDVPDYAS [SEQ ID NO: 790]

TABLE 176
ET200-026

DNA Sequence
aattttatgctgactcagccccactctgtgtcggagtctccggggaagac
ggtaaccatctcctgcaccggcagcagtggcagcattgccagcaactatg
tgcagtggtaccagcagcgcccgggcagtgcccccaccactgtgatctat
gaggataaccaaagaccctctggggtccctgatcggttctctggctccat
cgacagctcctccaactctgcctccctcaccatctctggactgaagactg
aggacgaggctgactactactgtcagtcttatgatagcagcaattgggtg
ttcggcggagggaccaagctgaccgtcctaggttctagaggtggtggtgg
tagcggcggcggcggctctggtggtggtggatccctcgagatggccgagg
tccagctggtgcagtctggggctgaggtgaagaagcctgggtcctcggtg
aaggtctcctgcaaggcttctggaggcaccttcagcagctatgctatcag
ctgggtgcgacaggcccctggacaagggcttgagtggatgggagggatca
tccctatctttggtacagcaaactacgcacagaagttccagggcagagtc
acgattaccgcggacgaatccacgagcacagcctacatggagctgagcag
cctgagatctgaggacacggccgtgtattactgtgcgcgcaacaaccatt
actacaacgattactggggtcaaggtactctggtgaccgtctcctcaact
agtggccaggccggccagcaccatcaccatcaccatggcgcatacccgta
cgacgttccggactacgcttct [SEQ ID NO: 791]
Amino Acid Sequence
NFMLTQPHSVSESPGKTVTISCTGSSGSIASNYVQWYQQRPGSAPTTVIY
EDNQRPSGVPDRFSGSIDSSSNSASLTISGLKTEDEADYYCQSYDSSNWV
FGGGTKLTVLGSRGGGGSGGGGSGGGGSLEMAEVQLVQSGAEVKKPGSSV
KVSCKASGGTFSSYAISWVRQAPGQGLEWMGGIIPIFGTANYAQKFQGRV
TITADESTSTAYMELSSLRSEDTAVYYCARNNHYYNDYWGQGTLVTVSST
SGQAGQHHHHHHGAYPYDVPDYAS [SEQ ID NO: 792]

TABLE 177
ET200-027

DNA Sequence
cagtctgtgttgacgcagccgccctcagtgtctggggccccagggcaggg
ggtcaccatcccctgcactgggagcagctccaacatcggggcaggttatg
atgtacactggtaccagcagcttccagggacagcccccaaactcctcatc
tatggtaacaacaatcggccctcaggggtccctgaccgcttctctggctc
caggtctggctcctcagcctccctggccatcactgggctccaggctgagg
atgaggctgattattactgccagtcctatgacagcagcctgagtgatgtg
gtattcggcggagggaccaaggtcaccgtcctaggttctagaggtggtgg
tggtagcggcggcggcggctctggtggtggtggatccctcgagatggccg
aggtccagctggtgcagtctggggctgaggtgaagaagcctggggctaca
gtgaaaatctcctgcaaggtttctggatacaccttcaccgactactacat
gcactgggtgcaacaggcccctggaaaagggcttgagtggatgggacttg
ttgatcctgaagatggtgaaacaatatacgcagagaagttccagggcaga
gtcaccataaccgcggacacgtctacagacacagcctacatggagctgag
cagcctgagatctgaggacacggccgtgtattactgtgcgcgctactggt
cttactctttcgactacctgtacatgccggaaggtaacgattggtggggt
caaggtactctggtgaccgtctcctcaactagtggccaggccggccagca
ccatcaccatcaccatggcgcatacccgtacgacgttccggactacgctt
ct [SEQ ID NO: 793]
Amino Acid Sequence
QSVLTQPPSVSGAPGQGVTIPCTGSSSNIGAGYDVHWYQQLPGTAPKLLI
YGNNNRPSGVPDRFSGSRSGSSASLAITGLQAEDEADYYCQSYDSSLSDV
VFGGGTKVTVLGSRGGGGSGGGGSGGGGSLEMAEVQLVQSGAEVKKPGAT
VKISCKVSGYTFTDYYMHWVQQAPGKGLEWMGLVDPEDGETIYAEKFQGR
VTITADTSTDTAYMELSSLRSEDTAVYYCARYWSYSFDYLYMPEGNDWWG
QGTLVTVSSTSGQAGQHHHHHHGAYPYDVPDYAS
[SEQ ID NO: 794]

TABLE 178
ET200-028

DNA Sequence
cagtctgtgttgactcagccacccgcagcgtctgggacccccggacagag
agtcaccatctcttgttctgggggcgtctccaacatcgggagtggtgctc
taaattggtaccagcaactcccaggaacggcccccaaactcctcatctat
agttacaatcagcggccctcaggggtctctgaccgattctctggctccag
gtctgccacctcagcctccctggccatcagtgggctccagtctgaggatg
aggctgattattactgtgcaacctgggatgatagtgtgaatggttgggtg
ttcggcggagggaccaagctgaccgtcctaggttctagaggtggtggtgg
tagcggcggcggcggctctggtggtggtggatccctcgagatggcccagg
tccagctggtacagtctggagctgaggtgaagaagcctggggattcagtg
aaggtctcctgcaagccttctggttacaattttctcaactatggtatcaa
ctgggtgcgacaggcccctggacaagggcttgagtggatgggatggatta
gcacttacaccggtaacacaaactatgcacagaagctgcagggcagagtc
accttcaccacagacacatccacgagcacagcctacatggagatgaggag
cctgagatctgacgacacggccgtgtattactgtgcgcgcgacctgtact
actacgaaggtgttgattactggggtcaaggtactctggtgaccgtctcc
tcaactagtggccaggccggccagcaccatcaccatcaccatggcgcata
cccgtacgacgttccggactacgcttct [SEQ ID NO: 795]
Amino Acid Sequence
QSVLTQPPAASGTPGQRVTISCSGGVSNIGSGALNWYQQLPGTAPKLLIY
SYNQRPSGVSDRFSGSRSATSASLAISGLQSEDEADYYCATWDDSVNGWV
FGGGTKLTVLGSRGGGGSGGGGSGGGGSLEMAQVQLVQSGAEVKKPGDSV
KVSCKPSGYNFLNYGINWVRQAPGQGLEWMGWISTYTGNTNYAQKLQGRV
TFTTDTSTSTAYMEMRSLRSDDTAVYYCARDLYYYEGVDYWGQGTLVTVS
STSGQAGQHHHHHHGAYPYDVPDYAS [SEQ ID NO: 796]

TABLE 179
ET200-029

DNA Sequence
caggctgtgctgactcagccaccctcagtgtcagtggccccaggaaagac
ggccagggttacctgtgggggaaacaacattggaagtgaaagtgtgcact
ggtaccagcagaagccaggccaggcccctgtgttggtcatctattatgat
accgaccggccctcagggatccctgagcgattctctggctcccactctgg
gaccacggccaccctgaccatcagcagggtcgaagccggggatgaggccg
actattactgtcaggtgtgggatagtagtagggatcatgtggtattcggc
ggagggaccaagctgaccgtcctaggttctagaggtggtggtggtagcgg
cggcggcggctctggtggtggtggatccctcgagatggcccaggtgcagc
tggtgcagtctgggggaggcgtggtccagcctgggaggtccctgagactc
tcctgtgcggcctctggattcaccttcagtagctatgctatgcactgggt
ccgccaggctccaggcaagggactggagtgggtggcagttatatcatatg
atggaagcaataaatactacgcagactccgtgaagggcctattcaccatc
tccagagacaattccaagaacacgctgtatctgcaaatgaacagcctgag
agctgaggacacggccgtgtattactgtgcgcgctcttacttcacttctg
gtttctacgattactggggtcaaggtactctggtgaccgtctcctcaact
agtggccaggccggccagcaccatcaccatcaccatggcgcatacccgta
cgacgttccggactacgcttct [SEQ ID NO: 797]
Amino Acid Sequence
QAVLTQPPSVSVAPGKTARVTCGGNNIGSESVHWYQQKPGQAPVLVIYYD
TDRPSGIPERFSGSHSGTTATLTISRVEAGDEADYYCQVWDSSRDHVVFG
GGTKLTVLGSRGGGGSGGGGSGGGGSLEMAQVQLVQSGGGVVQPGRSLRL
SCAASGFTFSSYAMHWVRQAPGKGLEWVAVISYDGSNKYYADSVKGLFTI
SRDNSKNTLYLQMNSLRAEDTAVYYCARSYFTSGFYDYWGQGTLVTVSST
SGQAGQHHHHHHGAYPYDVPDYAS [SEQ ID NO: 798]

TABLE 180
ET200-030

DNA Sequence
cagtctgtcgtgacgcagccgccctcagtgtctggggccccagggcagagggtcaccatctcctgcactgggagcagttccaa
catcggggcaggttatgatgtaaattggtatcagcagtttccaggaacagcccccaaactcctcatctatggtaacagcaatcggc
cctcaggggtccctgaccgattctctggctccaagtctggcacctcagcctccctggccatcactgggctccaggctgaggatg
aggctgattattactgccagtcctatgacagcagcctgagtggctcttatgtcttcggaactgggaccaaggtcaccgtcctaggtt
ctagaggtggtggtggtagcggcggcggcggctctggtggtggtggatccctcgagatggcccagatgcagctggtgcagtct
ggggctgaggtgaagaagcctggggcctcagtgaaggtctcctgcaaggcttccggatacaccctcactgaattatccatgcac
tgggtgcgacaggctcctggaaaagggcttgagtggatgggaggttttgatcctgaagatggtgaaacaatctacgcacagaag
ttccagggcagagtcaccatgaccgaggacacatctacagacacagcctacatggagctgagcagcctgagatctgaggaca
ctgccgtgtattactgtgcgcgcatgtcttctatgtactacgattggggtcaaggtactctggtgaccgtctcctcaactagtggcca
ggccggccagcaccatcaccatcaccatggcgcatacccgtacgacgttccggactacgcttct [SEQ ID NO: 799]
Amino Acid Sequence
QSVVTQPPSVSGAPGQRVTISCTGSSSNIGAGYDVNWYQQFPGTAPKLLIYGNSN
RPSGVPDRFSGSKSGTSASLAITGLQAEDEADYYCQSYDSSLSGSYVFGTGTKVT
VLGSRGGGGSGGGGSGGGGSLEMAQMQLVQSGAEVKKPGASVKVSCKASGYT
LTELSMHWVRQAPGKGLEWMGGFDPEDGETIYAQKFQGRVTMTEDTSTDTAY
MELSSLRSEDTAVYYCARMSSMYYDWGQGTLVTVSSTSGQAGQHHHHHHGAY
PYDVPDYAS [SEQ ID NO: 800]

TABLE 181
ET200-031

DNA Sequence
tcctatgtgctgactcagccaccctcagtgtcagtggccccaggaaagac
ggccaggattacctgtgggggaaacaacattggaagtaaaagtgtgcact
ggtaccagcagaagccaggccaggcccctgtgctggtcatctattatgat
agcgaccggccctcagggatccctgagcgattctctggctccaactctgg
gaacacggccaccctgaccatcagcagggtcgaagccggggatgaggccg
actattactgtcaggtgtgggatagtagtagtgattatgtcttcggaact
gggaccaaggtcaccgtcctaggttctagaggtggtggtggtagcggcgg
cggcggctctggtggtggtggatccctcgagatggccgaggtgcagctgg
tggagactgggggaggcttggtcaagcctggagggtccctgagactctcc
tgtgcagcctctggattcaccgtcagtgactactacatgagctggatccg
ccaggctccagggaagggcctggagtggatttcatacattagtggtagtg
gtaatagcatatactacgcagactctgtgaagggccgattcaccatctcc
agggacaacgccaagaactcactggatctgcaaatgaccagcctgagagc
cgaggacacggccgtatattactgtgcgcgctctactaaattcgattact
ggggtcaaggtactctggtgaccgtctcctcaactagtggccaggccggc
cagcaccatcaccatcaccatggcgcatacccgtacgacgttccggacta
cgcttct [SEQ ID NO: 801]
Amino Acid Sequence
SYVLTQPPSVSVAPGKTARITCGGNNIGSKSVHWYQQKPGQAPVLVIYYD
SDRPSGIPERFSGSNSGNTATLTISRVEAGDEADYYCQVWDSSSDYVFGT
GTKVTVLGSRGGGGSGGGGSGGGGSLEMAEVQLVETGGGLVKPGGSLRLS
CAASGFTVSDYYMSWIRQAPGKGLEWISYISGSGNSIYYADSVKGRFTIS
RDNAKNSLDLQMTSLRAEDTAVYYCARSTKFDYWGQGTLVTVSSTSGQAG
QHHHHHHGAYPYDVPDYAS [SEQ ID NO: 802]

TABLE 182
ET200-032

DNA Sequence
ctgcctgtgctgactcagccaccctcagcgtctgggacccccgggcagag
ggtcaccatctcttgttctggaagcagctccaacgtcggaagttacactg
taaactggtaccggcaactcccaggaacggcccccacactcctcatctat
aataataatcagcggccctcaggggtccctgaccgattctctgactccaa
gtctggcacctcggcctccctgaccattagtgggctccagcctgaggatg
aggctgattattattgtgcagcatgggatgacaggctgggtggttatgtc
ttcggaactgggaccaaggtcaccgtcctaggttctagaggtggtggtgg
tagcggcggcggcggctctggtggtggtggatccctcgagatggccgagg
tgcagctggtgcagtctggagcagaggtgaaaaagccgggggagtctctg
aagatctcctgtaagggttctggatacagctttaccaactactggatcgg
ctgggtgcgccagatgcccgggaaaggcctggagtggatggggatcatct
atcctggtgactctgataccagatacagcccgtccttccaaggccaggtc
accatctcagccgacaagtccatcagcaccgcctacctacagtggagcag
cctgaaggcctcggacaccgccatgtattactgtgcgcgctctactggtt
cttctcatatgtctgatgaatggggtcaaggtactctggtgaccgtctcc
tcaactagtggccaggccggccagcaccatcaccatcaccatggcgcata
cccgtacgacgttccggactacgcttct [SEQ ID NO: 803]
Amino Acid Sequence
LPVLTQPPSASGTPGQRVTISCSGSSSNVGSYTVNWYRQLPGTAPTLLIY
NNNQRPSGVPDRFSDSKSGTSASLTISGLQPEDEADYYCAAWDDRLGGYV
FGTGTKVTVLGSRGGGGSGGGGSGGGGSLEMAEVQLVQSGAEVKKPGESL
KISCKGSGYSFTNYWIGWVRQMPGKGLEWMGIIYPGDSDTRYSPSFQGQV
TISADKSISTAYLQWSSLKASDTAMYYCARSTGSSHMSDEWGQGTLVTVS
STSGQAGQHHHHHHGAYPYDVPDYAS [SEQ ID NO: 804]

TABLE 183
ET200-033

DNA Sequence
aattttatgctgactcagccccactctgtgtcggagtctccggggaagac
ggtaaccatctcctgcaccggcagcagtggcagcattgccagcaactatg
tgcagtggtaccagcagcgcccgggcagtgcccccaccactgtgatctat
gaggataaccaaagaccctctggggtccctgatcggttctctggctccat
cgacagctcctccaactctgcctccctcaccatctctggactgaagactg
aggacgaggctgactactactgtcagtcttatgatagcagcaatcattgg
gtgttcggcggagggaccaagctgaccgtcctaggttctagaggtggtgg
tggtagcggcggcggcggctctggtggtggtggatccctcgagatggccc
aagtgcagctacagcagtggggcgcaggactgttgaagccttcggagacc
ctgtccctcacctgcgctgtctatggtgggtccttcagtggttactactg
gagctggatccgccagcccccagggaaggggctggagtggattggggaga
tcactcatagtggaaggtccaactacaacccgtccctcaagagtcgagtc
accatatcagtagacacgtccaagaaccagttctccctgaagctgagctc
tgtgaccgccgcggacacggccgtgtattactgtgcgcgctcttctatca
tgtctgattactggggtcaaggtactctggtgaccgtctcctcaactagt
ggccaggccggccagcaccatcaccatcaccatggcgcatacccgtacga
cgttccggactacgcttct [SEQ ID NO: 805]
Amino Acid Sequence
NFMLTQPHSVSESPGKTVTISCTGSSGSIASNYVQWYQQRPGSAPTTVIY
EDNQRPSGVPDRFSGSIDSSSNSASLTISGLKTEDEADYYCQSYDSSNHW
VFGGGTKLTVLGSRGGGGSGGGGSGGGGSLEMAQVQLQQWGAGLLKPSET
LSLTCAVYGGSFSGYYWSWIRQPPGKGLEWIGEITHSGRSNYNPSLKSRV
TISVDTSKNQFSLKLSSVTAADTAVYYCARSSIMSDYWGQGTLVTVSSTS
GQAGQHHHHHHGAYPYDVPDYAS [SEQ ID NO: 806]

TABLE 184
ET200-034

DNA Sequence
cagtctgtgttgacgcagccgccctcagtgtctggggccccagggcagag
ggtcaccatctcctgcactgggagcacctccaacatcggggcaggttatg
atgtacactggtaccagcagcttccaggaacagcccccaaactcctcatc
aacaataacaggaatcggccctcaggggtccctgaccgattctctggctc
caagtctggcacgtcagccaccctgggcatcaccggactccagactgggg
acgaggccgattattactgcggaacatgggatggcagcctgactggtgca
gtgttcggcggagggaccaagctgaccgtcctaggttctagaggtggtgg
tggtagcggcggcggcggctctggtggtggtggatccctcgagatggccg
aggtccagctggtgcagtctggggctgaggtgaagaagcctgggtcctcg
tggaaggtctcatgcaaggcttctggaggcaccttcagcagctatgctat
cagctgggtgcgacaggcccctggacaagggcttgagtggatgggaggga
tcatccctatctttggtacagcaaactacgcacagaagttccagggcaga
gtcacgattaccgcggacgaatccacgagcacagcctacatggagctgag
cagcctgagatctgaggacacggccgtgtattactgtgcgcgcggttctg
ctctggaccattacgatcgttggggtcaaggtactctggtgaccgtctcc
tcaactagtggccaggccggccagcaccatcaccatcaccatggcgcata
cccgtacgacgttccggactacgcttct [SEQ ID NO: 807]
Amino Acid Sequence
QSVLTQPPSVSGAPGQRVTISCTGSTSNIGAGYDVHWYQQLPGTAPKLLI
NNNRNRPSGVPDRFSGSKSGTSATLGITGLQTGDEADYYCGTWDGSLTGA
VFGGGTKLTVLGSRGGGGSGGGGSGGGGSLEMAEVQLVQSGAEVKKPGSS
VKVSCKASGGTFSSYAISWVRQAPGQGLEWMGGIIPIFGTANYAQKFQGR
VTITADESTSTAYMELSSLRSEDTAVYYCARGSALDHYDRWGQGTLVTVS
STSGQAGQHHHHHHGAYPYDVPDYAS [SEQ ID NO: 808]

TABLE 185
ET200-035

DNA Sequence
aattttatgctgactcagccccactctgtgtcggagtctccggggaagac
ggtaaccatctcctgcacccgcagcagtggcagcattgccagcaactatg
tgcagtggtaccagcagcgcccgggcagtgcccccaccactgtgatctat
gaggataaccaaagaccctctggggtccctgatcggttctctggctccat
cgacagctcctccaactctgcctccctcaccatctctggactgaagactg
aggacgaggctgactactactgtcagtcttatgatagcaccaattgggtg
ttcggcggagggaccaagctgaccgtcctaggttctagaggtggtggtgg
tagcggcggcggcggctctggtggtggtggatccctcgagatggcccagg
tgcagctggtgcagtctggggctgaggtgaagaagcctgggtcctcggtg
aaggtctcctgcaaggcttctggaggcaccttcagcagctatgctatcag
ctgggtgcgacaggcccctggacaagggcttgagtggatgggagggatca
tccctatctttggtacagcaaactacgcacagaagttccagggcagagtc
acgattaccgcggacgaatccacgagcacagcctacatggagctgagcag
cctgagatctgaggacactgccgtgtattactgtgcgcgctacaactact
acttcaacgattactggggtcaaggtactctggtgaccgtctcctcaact
agtggccaggccggccagcaccatcaccatcaccatggcgcatacccgta
cgacgttccggactacgcttct [SEQ ID NO: 809]
Amino Acid Sequence
NFMLTQPHSVSESPGKTVTISCTRSSGSIASNYVQWYQQRPGSAPTTVIY
EDNQRPSGVPDRFSGSIDSSSNSASLTISGLKTEDEADYYCQSYDSTNWV
FGGGTKLTVLGSRGGGGSGGGGSGGGGSLEMAQVQLVQSGAEVKKPGSSV
KVSCKASGGTFSSYAISWVRQAPGQGLEWMGGIIPIFGTANYAQKFQGRV
TITADESTSTAYMELSSLRSEDTAVYYCARYNYYFNDYWGQGTLVTVSST
SGQAGQHHHHHHGAYPYDVPDYAS [SEQ ID NO: 810]

TABLE 186
ET200-037

DNA Sequence
tcctatgtgctgactcagccaccctcagtgtcagtggccccaggaaagac
ggccaggattacctgtgggggaaacaacattggaagtaaaagtgtgcact
ggtaccagcagaagccaggccaggcccctgtgctggtcatctattatgat
agcgaccggccctcagggatccctgagcgattctctggctccaactctgg
gaacacggccaccctgaccatcagcagggtcgaagccggggatgaggccg
actattactgtcaggtgtgggatagtagtagtgatcatccttatgtcttc
ggaactgggaccaaggtcaccgtcctaggttctagaggtggtggtggtag
cggcggcggcggctctggtggtggtggatccctcgagatggcccagatgc
agctggtgcagtctggagctgaggtgaagaagcctggggcctcagtgaag
gtctcctgcaaggcttctggttacacctttaccagctatggtatcagctg
ggtgcgacaggcccctggacaagggcttgagtggatgggatggatcagcg
cttacaatggtaacacaaactatgcacagaagctccagggcagagtcacc
atgaccacagacacatccacgagcacagcctacatggagctgaggagcct
gagatctgacgacactgccgtgtattactgtgcgcgctctatgttcggtg
ctcatgattcttggggtcaaggtactctggtgaccgtctcctcaactagt
ggccaggccggccagcaccatcaccatcaccatggcgcatacccgtacga
cgttccggactacgcttct [SEQ ID NO: 811]
Amino Acid Sequence
SYVLTQPPSVSVAPGKTARITCGGNNIGSKSVHWYQQKPGQAPVLVIYYD
SDRPSGIPERFSGSNSGNTATLTISRVEAGDEADYYCQVWDSSSDHPYVF
GTGTKVTVLGSRGGGGSGGGGSGGGGSLEMAQMQLVQSGAEVKKPGASVK
VSCKASGYTFTSYGISWVRQAPGQGLEWMGWISAYNGNTNYAQKLQGRVT
MTTDTSTSTAYMELRSLRSDDTAVYYCARSMFGAHDSWGQGTLVTVSSTS
GQAGQHHHHHHGAYPYDVPDYAS [SEQ ID NO: 812]

TABLE 187
ET200-038

DNA Sequence
cagtctgtgttgacgcagccgccctcagtgtctggggccccagggcagag
ggtcaccatctcctgcactgggagcagctccaacatcggggcaggttttg
atgtacactggtaccagctacttccaggaacagcccccaaactcctcatc
tatgctaacagcaatcggccctcaggggtccctgaccgattctctggctc
caagtctggcacctcagcctccctggccatcactgggctcctggctgagg
atgaggctgattattactgccagtcctatgacagcagcctgagtggtgtg
gtattcggcggagggaccaagctgaccgtcctaggttctagaggtggtgg
tggtagcggcggcggcggctctggtggtggtggatccctcgagatggccc
aggtgcagctggtgcaatctggggctgaggtgaagaagcctgggtcctcg
gtgaaggtctcctgcaaggcttctggaggcaccttcagcagctatgctat
cagctgggtgcgacaggcccctggacaagggcttgagtggatgggaggga
tcatccctatctttggtacagcaaactacgcacagaagttccagggcaga
gtcacgattaccgcggacgaatccacgagcacagcctacatggagctgag
cagcctgagatctgaggacactgccgtgtattactgtgcgcgcggtgctt
ctttcgaccgtcatgataactggggtcaaggtactctggtgaccgtctcc
tcaactagtggccaggccggccagcaccatcaccatcaccatggcgcata
cccgtacgacgttccggactacgcttct [SEQ ID NO: 813]
Amino Acid Sequence
QSVLTQPPSVSGAPGQRVTISCTGSSSNIGAGFDVHWYQLLPGTAPKLLI
YANSNRPSGVPDRFSGSKSGTSASLAITGLLAEDEADYYCQSYDSSLSGV
VFGGGTKLTVLGSRGGGGSGGGGSGGGGSLEMAQVQLVQSGAEVKKPGSS
VKVSCKASGGTFSSYAISWVRQAPGQGLEWMGGIIPIFGTANYAQKFQGR
VTITADESTSTAYMELSSLRSEDTAVYYCARGASFDRHDNWGQGTLVTVS
STSGQAGQHHHHHHGAYPYDVPDYAS [SEQ ID NO: 814]

TABLE 188
ET200-039

DNA Sequence
aattttatgctgactcagccccactctgtgtcggagtctccggggaagac
ggtaaccatctcctgcacccgcagcagtggcagcattgccagcaactatg
tgcagtggtaccagcagcgcccgggcagttcccccaccactgtgatctat
gaggataaccaaagaccctctggggtccctgatcggttctctggctccat
cgacagctcctccaactctgcctccctcaccatctctggactgaagactg
aggacgaggctgactactactgtcagtcttatgatagcagcaattgggtg
ttcggcggagggaccaagctgaccgtcctaggttctagagtggtggtggt
agcggcggcggcggctctggtggtggtggatccctcgagatggccgaggt
ccagctggtgcagtctggggctgaggtgaagaagcctgggtcctcggtga
aggtctcctgcaaggcttctggaggcaccttcagcagctatgctatcagc
tgggtgcgacaggcccctggacaagggcttgagtggatgggagggatcat
ccctatctttggtacagcaaactacgcacagaagttccagggcagagtca
cgattaccgcggacgaatccacgagcacagcctacatggagctgagcagc
ctgagatctgaggacacggccgtgtattactgtgcgcgctctaactacta
ctacaacgattactggggtcaaggtactctggtgaccgtctcctcaacta
gtggccaggccggccagcaccatcaccatcaccatggcgcatacccgtac
gacgttccggactacgcttct [SEQ ID NO: 815]
Amino Acid Sequence
NFMLTQPHSVSESPGKTVTISCTRSSGSIASNYVQWYQQRPGSSPTTVIY
EDNQRPSGVPDRFSGSIDSSSNSASLTISGLKTEDEADYYCQSYDSSNWV
FGGGTKLTVLGSRGGGGSGGGGSGGGGSLEMAEVQLVQSGAEVKKPGSSV
KVSCKASGGTFSSYAISWVRQAPGQGLEWMGGIIPIFGTANYAQKFQGRV
TITADESTSTAYMELSSLRSEDTAVYYCARSNYYYNDYWGQGTLVTVSST
SGQAGQHHHHHHGAYPYDVPDYAS [SEQ ID NO: 816]

TABLE 189
ET200-040

DNA Sequence
cagtctgtgttgacgcagccgccctcagtgtctggggccccagggcagag
ggtcaccatctcctgcactgggagcagctccaacatcggggcaggttatg
atgtacactggtaccagcagcttccaggaacagcccccaaactcctcatc
tatggtaacagcaatcggccctcaggggtccctgaccgattctctggctc
caagtctggcacctcagcctccctggccatcactgggctccaggctgagg
atgaggctgattattactgccagtcctatgacagcagcctgagtggttat
gtcttcggaactgggaccaaggtcaccgtcctaggttctagaggtggtgg
tggtagcggcggcggcggctctggtggtggtggatccctcgagatggccc
aggtgcagctggtgcagtctggggctgaggtgaagaagcctggggcctca
gtgaaggtctcctgcaaggtttccggatacaccctcactgaattatccat
gcactgggtgcgacaggctcctggaaaagggcttgagtggatgggaggtt
ttgatcctgaagatggtgaaacaatctacgcacagaagttccagggcaga
gtcaccatgaccgaggacacatctacagacacagcctacatggagctgag
cagcctgagatctgaggacactgccgtgtattactgtgcgcgctactctg
gtgtttactacgattggggtcaaggtactctggtgaccgtctcctcaact
agtggccaggccggccagcaccatcaccatcaccatggcgcatacccgta
cgacgttccggactacgcttct [SEQ ID NO: 817]
Amino Acid Sequence
QSVLTQPPSVSGAPGQRVTISCTGSSSNIGAGYDVHWYQQLPGTAPKLLI
YGNSNRPSGVPDRFSGSKSGTSASLAITGLQAEDEADYYCQSYDSSLSGY
VFGTGTKVTVLGSRGGGGSGGGGSGGGGSLEMAQVQLVQSGAEVKKPGAS
VKVSCKVSGYTLTELSMHWVRQAPGKGLEWMGGFDPEDGETIYAQKFQGR
VTMTEDTSTDTAYMELSSLRSEDTAVYYCARYSGVYYDWGQGTLVTVSST
SGQAGQHHHHHHGAYPYDVPDYAS [SEQ ID NO: 818]

TABLE 190
ET200-041

DNA Sequence
aattttatgctgactcagccccactctgtgtcggggtctccggggaagac
ggtaaccatctcctgcaccggcagcagtggcagcattgccgacaactttg
tgcagtggtaccagcagcgcccgggcggtgtccccaccactgtgatcttt
aatgatgacgaaagaccctctggcgtccctgatcggttctctggctccat
cgacacctcctccaattctgcctccctcaccatctctggactgaagactg
aggacgggctgactactactgtcagtcttatgataataataatcgagggg
tgttcggcggagggaccaagctgaccgtcctaggttctagaggtggtggt
ggtagcggcggcggcggctctggtggtggtggatccctcgagatggccca
ggtccagctggtgcagtctggggctgaggtgaagaagcctgggtcctcgg
tgaaggtctcctgcaaggcttctggaggcaccttcagcagctatgctatc
agctgggtgcgacaggcccctggacaagggcttgagtggatgggatggat
gaaccctaacagtggtaacacaggctatgcacagaagttccagggcagag
tcaccatgaccaggaacacctccataagcacagcctacatggagctgagc
aacctgagatctgaggacacggccgtgtattactgtgcgcgctactactc
ttacggttacgattggggtcaaggtactctggtgaccgtctcctcaacta
gtggccaggccggccagcaccatcaccatcaccatggcgcatacccgtac
gacgttccggactacgcttct [SEQ ID NO: 819]
Amino Acid Sequence
NFMLTQPHSVSGSPGKTVTISCTGSSGSIADNFVQWYQQRPGGVPTTVIF
NDDERPSGVPDRFSGSIDTSSNSASLTISGLKTEDEADYYCQSYDNNNRG
VFGGGTKLTVLGSRGGGGSGGGGSGGGGSLEMAQVQLVQSGAEVKKPGSS
VKVSCKASGGTFSSYAISWVRQAPGQGLEWMGWMNPNSGNTGYAQKFQGR
VTMTRNTSISTAYMELSNLRSEDTAVYYCARYYSYGYDWGQGTLVTVSST
SGQAGQHHHHHHGAYPYDVPDYAS [SEQ ID NO: 820]

TABLE 191
ET200-042

DNA Sequence
cagtctgtcgtgacgcagccgccctcagtgtctggggccccagggcagac
ggtcaccatctcctgcactgggggcagctccaacatcgggacaggttatt
ttgtaaattggtaccagcaggttccaggaaaagcccccaaactcctcatc
ctgggtaacaataatcggccctcgggggtccctgaccgactctccggctc
cacgtccggcacctcagcctccctggccatcactgggctccaggctgagg
atgagggtacttattactgccagtcctatgacagcagcctgagtggttat
gtcttcggaactgggaccaaggtcaccgtcctaggttctagaggtggtgg
tggtagcggcggcggcggctctggtggtggtggatccctcgagatggccc
aggtacagctgcagcagtcaggtccaggactggtgaagccctcgcagacc
ctctcactcacctgtggcatctccggggacagtgtctctaccaacagtgt
tgcttggcactggatcaggcagtccccatcgagaggccttgagtggctgg
gaaggacatactacaggtccaagtggtctaatgactatggagtatctgtg
aaaagtcgaatcaccatcatcccagacacatccaagaaccagttctccct
gcagctgaactctgtgactcccgaggacacggctgtgtattactgtgcgc
gctcttcttcttggtaccagatcttcgattactggggtcaaggtactctg
gtgaccgtctcctcaactagtggccaggccggccagcaccatcaccatca
ccatggcgcatacccgtacgacgttccggactacgcttct
[SEQ ID NO: 821]
Amino Acid Sequence
QSVVTQPPSVSGAPGQTVTISCTGGSSNIGTGYFVNWYQQVPGKAPKLLI
LGNNNRPSGVPDRLSGSTSGTSASLAITGLQAEDEGTYYCQSYDSSLSGY
VFGTGTKVTVLGSRGGGGSGGGGSGGGGSLEMAQVQLQQSGPGLVKPSQT
LSLTCGISGDSVSTNSVAWHWIRQSPSRGLEWLGRTYYRSKWSNDYGVSV
KSRITIIPDTSKNQFSLQLNSVTPEDTAVYYCARSSSWYQIFDYWGQGTL
VTVSSTSGQAGQHHHHHHGAYPYDVPDYAS [SEQ ID NO: 822]

TABLE 192
ET200-043

DNA Sequence
aattttatgctgactcagccccactctgtgtcggagtctccggggaagac
ggtaaccatctcctgcaccggcagcagcgacagcatagccaacaactatg
ttcagtggtaccagcagcgcccgggcagtgcccccaccaatgtgatctac
gaagatgtccaaagaccctctggggtccctgatcggttctctgggtccat
cgacagctcctccaactctgcctccctcaccatctctggactgaagactg
aggacgaggctgtctactattgtcagtcttatcatagcgacaatcgttgg
gtgttcggcggcgggaccaagctgaccgtcctaggttctagaggtggtgg
tggtagcggcggcggcggctctggtggtggtggatccctcgagatggccc
aggtgcagctggtggagtctgggggaggcttggtacagcctggggggtcc
ctgagactctcctgtgcagcctctggattcacctttagcagctatgccat
gagctgggtccgccaggctccagggaaggggctggagtgggtctcagcta
ttagtggtagtggtggtagcacatactacgcagactccgtgaagggccgg
ttcaccatctccagagacaattccaagaacacgctgtatctgcaaatgaa
cagcctgagagccgaggacacggccgtatattactgtgcgcgctctggtg
cttactgggactactctgtttacgatgaatggggtcaaggtactctggtg
accgtctcctcaactagtggccaggccggccagcaccatcaccatcacca
tggcgcatacccgtacgacgttccggactacgcttct
[SEQ ID NO: 823]
Amino Acid Sequence
NFMLTQPHSVSESPGKTVTISCTGSSDSIANNYVQWYQQRPGSAPTNVIY
EDVQRPSGVPDRFSGSIDSSSNSASLTISGLKTEDEAVYYCQSYHSDNRW
VFGGGTKLTVLGSRGGGGSGGGGSGGGGSLEMAQVQLVESGGGLVQPGGS
LRLSCAASGFTFSSYAMSWVRQAPGKGLEWVSAISGSGGSTYYADSVKGR
FTISRDNSKNTLYLQMNSLRAEDTAVYYCARSGAYWDYSVYDEWGQGTLV
TVSSTSGQAGQHHHHHHGAYPYDVPDYAS [SEQ ID NO: 824]

TABLE 193
ET200-044

DNA Sequence
cagtctgtgttgactcagccaccctcagtgtccgtgtccccaggacagac
agccaccatcgcctgttctggacataaattgggggataaatatgcttcct
ggtatcagcagaagtcgggccagtcccctgtgttgatcatctatcaggat
aataagcggccctcagggattcctgagcgattctctggctccaactctgg
gaacacagccactctgaccatcagcgggacccaggctctggatgaggctg
actattattgtcaggcgtgggacagtagtacttatgtggcattcggcgga
gggaccaagctgaccgtcctaggttctagaggtggtggtggtagcggcgg
cggcggctctggtggtggtggatccctcgagatggcccaggtgcagctgc
aggagtccggcccaggactggtgaagccttcggagaccctgtccctcacc
tgcgttgtctctggtggctccatcagcagtagtaactggtggagctgggt
ccgccagcccccagggaaggggctggagtggattggggaaatctatcata
gtgggagccccaactacaacccatccctcaagagtcgagtcaccatatca
gtagacaagtccaagaaccagttctccctgaagctgagctctgtgaccgc
cgcggacacggccgtgtattactgtgcgcgcatgactactcatactttcg
gttacgatgcttggggtcaaggtactctggtgaccgtctcctcaactagt
ggccaggccggccagcaccatcaccatcaccatggcgcatacccgtacga
cgttccggactacgcttct [SEQ ID NO: 825]
Amino Acid Sequence
QSVLTQPPSVSVSPGQTATIACSGHKLGDKYASWYQQKSGQSPVLIIYQD
NKRPSGIPERFSGSNSGNTATLTISGTQALDEADYYCQAWDSSTYVAFGG
GTKLTVLGSRGGGGSGGGGSGGGGSLEMAQVQLQESGPGLVKPSETLSLT
CVVSGGSISSSNWWSWVRQPPGKGLEWIGEIYHSGSPNYNPSLKSRVTIS
VDKSKNQFSLKLSSVTAADTAVYYCARMTTHTFGYDAWGQGTLVTVSSTS
GQAGQHHHHHHGAYPYDVPDYAS [SEQ ID NO: 826]

TABLE 194
ET200-045

DNA Sequence
cagcctgtgctgactcagccaccctcagtgtcagtggccccaggaaagac
ggccacgattacttgtgggggaaacaacattggaagtgaaagtgtgcact
ggtaccaccagaagccaggccaggcccctgtgttggtcatctatgatgat
gccggccggccctcagggatccctgagcgattcactggctccaactctgg
gaacacggccaccctgaccatcagcagggtcgaagccggggatgaggcga
ctattactgtcaggtgtgggacagaaatagctgctcagtttgtcttcgga
cctgggaccaaggtcaccgtcctaggttctagaggtggtggtggtagcgg
cggcggcggctctggtggtggtggatccctcgagatggccgaggtccagc
tggtgcagtctggagctgaggtgaagaagcctggggcctcagtgaaggtc
tcctgcaaggcttctggttacacctttaccagctatggtatcagctgggt
gcgacaggcccctggacaagggcttgagtggatgggatggatcagcgctt
acaatggtaacacaaactatgcacagaagctccagggcagagtcaccatg
accacagacacatccacgagcacagcctacatggagctgaggagcctgag
atctgacgacacggccgtgtattactgtgcgcgcggtgttcatctggatt
ggtggggtcaaggtactctggtgaccgtctcctcaactagtggccaggcc
ggccagcaccatcaccatcaccatggcgcatacccgtacgacgttccgga
ctacgcttct [SEQ ID NO: 827]
Amino Acid Sequence
QPVLTQPPSVSVAPGKTATITCGGNNIGSESVHWYHQKPGQAPVLVIYDD
AGRPSGIPERFTGSNSGNTATLTISRVEAGDEADYYCQVWDRNSAQFVFG
PGTKVTVLGSRGGGGSGGGGSGGGGSLEMAEVQLVQSGAEVKKPGASVKV
SCKASGYTFTSYGISWVRQAPGQGLEWMGWISAYNGNTNYAQKLQGRVTM
TTDTSTSTAYMELRSLRSDDTAVYYCARGVHLDWWGQGTLVTVSSTSGQA
GQHHHHHHGAYPYDVPDYAS [SEQ ID NO: 828]

TABLE 195
ET200-069

DNA Sequence
cagtctgtcgtgacgcagccaccctcagcgtctgggacccccgggcagag
ggtcaccatctcttgttctggaagcagctccaacatcggaagtaattatg
tatactggtaccagcagctcccaggaacggcccccaaactcctcatctat
agtaataatcagcggccctcaggggtccctgaccgattctctggctccaa
gtctggcacctcagcctccctggccatcagtgggctccggtccgaggatg
aggctgattattactgtgcagcatgggatgacagcctgagtggttatgtc
ttcggaactgggaccaagctgaccgtcctaggttctagaggtggtggtgg
tagcggcggcggcggctctggtggtggtggatccctcgagatggcccagg
tgcagctacagcagtggggcgcaggactgttgaagccttcggagaccctg
tccctcacctgcgctgtctatggtgggtccttcagtggttactactggag
ctggatccgccagcccccagggaaggggctggagtggattggggaaatca
atcatagtggaagcaccaactacaacccgtccctcaagagtcgagtcacc
atatcagtagacacgtccaagaaccagttctccctgaagctgagctctgt
gaccgccgcggacacggccgtgtattactgtgcgcgcctgtacgaaggtg
gttaccatggttggggttcttggctgtcttctgattcttggggtcaaggt
actctggtgacccgtctcctcaactagtggccaggccggccagcaccatc
accatcaccatggcgcatacccgtacgacgttccggactacgcttct
[SEQ ID NO: 829]
Amino Acid Sequence
QSVVTQPPSASGTPGQRVTISCSGSSSNIGSNYVYWYQQLPGTAPKLLIY
SNNQRPSGVPDRFSGSKSGTSASLAISGLRSEDEADYYCAAWDDSLSGYV
FGTGTKLTVLGSRGGGGSGGGGSGGGGSLEMAQVQLQQWGAGLLKPSETL
SLTCAVYGGSFSGYYWSWIRQPPGKGLEWIGEINHSGSTNYNPSLKSRVT
ISVDTSKNQFSLKLSSVTAADTAVYYCARLYEGGYHGWGSWLSSDSWGQG
TLVTVSSTSGQAGQHHHHHHGAYPYDVPDYAS [SEQ ID NO: 830]

TABLE 196
ET200-078

DNA Sequence
cagtctgtgttgactcagccaccctcagcgtctgggacccccgggcagag
ggtcaccatctcttgttctggaagcagctccaacatcggaagtaatactg
taaactggtaccagcagctcccaggaacggcccccaaactcctcatctat
agtaataatcagcggccctcggggtccctgaccgattctcatggctccaa
gtctggcacctcagcctccctggccatcagtgggctccagtctgaggatg
aggctattattactgtgcagcatgggatgacagcctggaatggttattgg
gtgttcggcggagggaccaagctgaccgtcctaggttctagaggtggtgg
tggtagcggcggcggcggctctggtggtggtggatccctcgagatggccc
aggtgcagctacagcagtggggcgcaggactgttgaagccttcggagacc
ctgtccctcacctgcgctgtctatggtgggtccttcagtggttactactg
gagctggatccgccagcccccagggaaggggctggagtggattggggaaa
tcaatcatagtggaagcaccaactacaacccgtccctcaagagtcgagtc
accatatcagtagacacgtccaagaaccagttctccctgaagctgagctc
tgtgaccgccgcggacacggctgtgtattactgtgcgcgcgaaggggcat
ttgatgcttttgatatctggggccaagggacaatggtcaccgtctcttca
actagtggccaggccggccagcaccatcaccatcaccatggcgcataccc
gtacgacgttccggactacgcttct [SEQ ID NO: 831]
Amino Acid Sequence
QSVLTQPPSASGTPGQRVTISCSGSSSNIGSNTVNWYQQLPGTAPKLLIY
SNNQRPSGVPDRFSGSKSGTSASLAISGLQSEDEADYYCAAWDDSLNGYW
VFGGGTKLTVLGSRGGGGSGGGGSGGGGSLEMAQVQLQQWGAGLLKPSET
LSLTCAVYGGSFSGYYWSWIRQPPGKGLEWIGEINHSGSTNYNPSLKSRV
TISVDTSKNQFSLKLSSVTAADTAVYYCAREGAFDAFDIWGQGTMVTVSS
TSGQAGQHHHHHHGAYPYDVPDYAS [SEQ ID NO: 832]

TABLE 197
ET200-079

DNA Sequence
tcctatgagctgactcagccaccctcagcgtctgggacccccgggcagag
ggtcaccatctcttgttctggaagcagctccaacatcggaagtaattatg
tatactggtaccagcagctcccaggaacggcccccaaactcttcatctat
aggaataatcagcggccctcaggggtccctgaccgattctctggctccaa
gtctggcacctcagcctccctggccatcagtgggctccggtccgaggatg
aggctgattattactgtgcagcatgggatgacagcctgagtggttatctc
ttcggaactgggaccaaggtcaccgtcctaggttctagaggtggtggtgg
tagcggcggcggcggctctggtggtggtggatccctcgagatggccgagg
tgcagctggtggagtctgggggaggcttggtacagcctggcaggtccctg
agactctcctgtgcagcctctggattcacctttgatgattatgccatgca
ctgggtccggcaagctccagggaagggcctggagtgggtctcaggtatta
gttggaatagtggtagcataggctatgcggactctgtgaagggccgattc
accatctccagagacaacgccaagaactccctgtatctgcaaatgaacag
tctgagagctgaggacacggccttgtattactgtgcaaatggcgactcca
actactactacggtatggacgtctggggccaagggaccacggtcaccgtc
tcctcaactagtggccaggccggccagcaccatcaccatcaccatggcgc
atacccgtacgacgttccggactacgcttct [SEQ ID NO: 833]
Amino Acid Sequence
SYELTQPPSASGTPGQRVTISCSGSSSNIGSNYVYWYQQLPGTAPKLFIY
RNNQRPSGVPDRFSGSKSGTSASLAISGLRSEDEADYYCAAWDDSLSGYL
FGTGTKVTVLGSRGGGGSGGGGSGGGGSLEMAEVQLVESGGGLVQPGRSL
RLSCAASGFTFDDYAMHWVRQAPGKGLEWVSGISWNSGSIGYADSVKGRF
TISRDNAKNSLYLQMNSLRAEDTALYYCANGDSNYYYGMDVWGQGTTVTV
SSTSGQAGQHHHHHHGAYPYDVPDYAS [SEQ ID NO: 834]

TABLE 198
ET200-081

DNA Sequence
cagtctgccctgactcagcctgcctccgtgtccgggtctcctggacagtc
gatcaccatctcctgcactggaaccagcagtgacattggtggttataact
atgtctcctggtaccaacaacacccaggcaaagcccccaaactcatgatt
tatgatgtcagtaatcggccctcaggggtttctaatcgcttctctggctc
caagtctggcaacacggcctccctgaccatctctgggctccaggctgagg
acgaggctgattattactgcatctcatatacacgcacctggaacccctat
gtcttcgggagtgggaccaaggtcaccgtcctaggttctagaggtggtgg
tggtagcggcggcggcggctctggtggtggtggatccctcgagatggccg
aggtgcagctggtgcagtctgggggaggcgtggtacagcctggggggtcc
ctgagactctcctgtgcagcctctggattcacctttgatgattatgccat
gcactgggtcctcaagctccagggaagggtctggagtgggtctctcttat
tagtggggatggtggtagcacatacgtatgcagactctgtgaagggccga
ttcaccatctccagagacaacagcaaaaactccctgtatctgcaaatgaa
cagtctgagaactgaggacaccgccttgtattactgtgcaaaagatcggg
cagcagctggctactactactacggtatggacgtctggggccaagggacc
acggtcaccgtctccttcaactagtggccaggccggccagcaccatcacc
atcaccatggcgcatacccgtacgacgttccggactacgcttct
[SEQ ID NO: 835]
Amino Acid Sequence
QSALTQPASVSGSPGQSITISCTGTSSDIGGYNYVSWYQQHPGKAPKLMI
YDVSNRPSGVSNRFSGSKSGNTASLTISGLQAEDEADYYCISYTRTWNPY
VFGSGTKVTVLGSRGGGGSGGGGSGGGGSLEMAEVQLVQSGGGVVQPGGS
LRLSCAASGFTFDDYAMHWVRQAPGKGLEWVSLISGDGGSTYYADSVKGR
FTISRDNSKNSLYLQMNSLRTEDTALYYCAKDRAAAGYYYYGMDVWGQGT
TVTVSSTSGQAGQHHHHHHGAYPYDVPDYAS [SEQ ID NO: 836]

TABLE 199
ET200-097

DNA Sequence
ctgcctgtgctgactcagccaccctcagtgtccgtgtccccaggacagac
agccatcatcacctgctctggagataaattgggggaaaaatatgtttcct
ggtatcagcagaagccaggccagtcccctgtactggtcatcgatcaagat
accaggaggccctcagggatccctgagcgattctctggctccaactctgg
gaccacagccactctgaccatcagcgggacccaggctatggatgaggctg
actattactgtcaggcgtgggacaggggtgtggtattcggcggagggacc
aagctgaccgtcctaggttctagaggtggtggtggtagcggcggcggcgg
ctctggtggtggtggatccctcgagatggccgaggtgcagctggtggagt
ctgggggagacttggtacagcctggcaggtccctgagactctcctgtgca
gcctctggattcacctttaatgattatgccatgcactgggtccggcaagc
tccagggaagggcctggagtgggtctcaggtattagttggagtggtaata
acataggctatgcggactctgtgaagggccgattcaccatctccagagac
aacgccaagaactccctgtatctgcaaatgaacagtctgagagctgagga
cacggccttgtattactgtgcaaaagatagtatacggtatggcatcacct
ggggaggttttgactactggggccagggaaccctggtcaccgtctcctca
actagtggccaggccggccagcaccatcaccatcaccatggcgcataccc
gtacgacgttccggactacgcttct [SEQ ID NO: 837]
Amino Acid Sequence
LPVLTQPPSVSVSPGQTAIITCSGDKLGEKYVSWYQQKPGQSPVLVIDQD
TRRPSGIPERFSGSNSGTTATLTISGTQAMDEADYYCQAWDRGVVFGGGT
KLTVLGSRGGGGSGGGGSGGGGSLEMAEVQLVESGGDLVQPGRSLRLSCA
ASGFTFNDYAMHWVRQAPGKGLEWVSGISWSGNNIGYADSVKGRFTISRD
NAKNSLYLQMNSLRAEDTALYYCAKDSIRYGITWGGFDYWGQGTLVTVSS
TSGQAGQHHHHHHGAYPYDVPDYAS [SEQ ID NO: 838]

TABLE 200
ET200-098

DNA Sequence
cagcctgtgctgactcagccaccctcggtgtccaagggcttgagacagaccgccacactcacctgcactgggaacagcaacaatgttggcaac
ctaggagtagcttggctgcagcagcaccagggccaccctcccaaactcctatcctacaggaataacaaccggccctcagggatctcagagagat
tatctgcatccaggtcaggaaacacagcctccctgaccattactggactccagcctgaggacgaggctgactattactgctcagcatgggacagt
agcctcagtgcttgggtgttcggcggagggaccaagctgaccgtcctaggttctagaggtggtggtggtagcggcggcggcggctctggtggt
ggtggatccctcgagatggccgaggtgcagctggtggagtctgggggagtcgtggtacagcctggggggtccctgagactctcctgtgcagcc
tctggattcacctttgatgattatgccatgcactgggtccgtcaagctccggggaagggtctggagtgggtctctcttattaattgggatggtggtag
cacctactatgcagactctgtgaagggtcgattcaccatctccagagacaacagcaaaaactccctgtatctgcaaatgaacagtctgagagctga
ggacaccgccttgtattactgtgcaaaagggatgggcctgagggcgtttgactactggggccagggaaccctggtcaccgtctcctcaactagtg
gccaggccggccagcaccatcaccatcaccatggcgcatacccgtacgacgttccggactacgcttct [SEQ ID NO: 839]
Amino Acid Sequence
QPVLTQPPSVSKGLRQTATLTCTGNSNNVGNLGVAWLQQHQGHPPKLLSYRNNNRPSGISE
RLSASRSGNTASLTITGLQPEDEADYYCSAWDSSLSAWVFGGGTKLTVLGSRGGGGSGGGG
SGGGGSLEMAEVQLVESGGVVVQPGGSLRLSCAASGFTFDDYAMHWVRQAPGKGLEWVS
LINWDGGSTYYADSVKGRFTISRDNSKNSLYLQMNSLRAEDTALYYCAKGMGLRAFDYW
GQGTLVTVSSTSGQAGQHHHHHHGAYPYDVPDYAS [SEQ ID NO: 840]

TABLE 201
ET200-099

DNA Sequence
cagtctgtgttgactcagccaccctcagcgtctgggacccccgggcagagggtcaccatctcctgttctggaagcagctcggaagtaa
tactgtaaactggtaccagcagctcccaggaacggcccccaaatcctcatctatagtaatgatcagcggccctcaggggtccctgaccgattctc
tggctccaagtccggcacctcagcctccctggccatcagtgggctccagtctgaggatgaggctgattattacttgtgcttcatgggatgacagcct
gaatggcgttatgtcttcggaactgggaccaaggtcaccgtcctaggttctagaggtggtggtggtagcggcggcggcggctctggtggtggtggt
ggatccctcgagatggcccaggtccagctggtacagtctggggctgaggtgaggaagcctggggcctcagtgaaggtttcctgaagacttctg
gatacaccttcagttggtatgctatacattgggtgcgccaggcccccggacaaaggcttgagtggatggatggatcaacgctggcaatggaa
cacaaaatattcacagaaatttcagggcagagtcagtcttaccagggacacatccgcgagcacagcctacatggagctgagcagcctgagatct
gatgacacggctgtgtattactgtgcgagacccgataattatggttcgggtggggatgtttttgatatctggggccaagggacaatggtcaccgtct
cttcaactagtggccaggccggccagcaccatcaccatcaccatcaccatggcgcatacccgtacgacgttccggactacgcttct [SEQ ID NO:
841]
Amino Acid Sequence
QSVLTQPPSASGTPGQRVTISCSGSSSNIGSNTVNWYQQLPGTAPKLLIYSNDQRPSGVPDRF
SGSKSGTSASLAISGLQSEDEADYYCASWDDSLNGRYVFGTGTKVTVLGSRGGGGSGGGGS
GGGGSLEMAQVQLVQSGAEVRKPGASVKVSCKTSGYTFSWYAIHWVRQAPGQRLEMWG
WINAGNGNTKYSQKFQGRVSLTRDTSASTAYMELSSLRSDDTAVYYCARPDNYGSGGDVF
DIWGQGTMVTSSTSGQAGQHHHHHHGAYPYDVPDYAS [SEQ ID NO: 842]

TABLE 202
ET200-100

DNA Sequence
aattttatgctgactcagccccactctgtgtcggagtctccggggaagacggtaaccatctcctgcacccgcagcagttgccagcaa
ctttgtgcagtggtaccagcagcgcccgggcagtgcccccacccctatgatctatgaggataacaacagaccccctggggtccctgatcggttct
ctcgcctccgtcgacagctcctccaactctgcctccctcaccatctctggactgaagactgaggacgaggctactactgtcagtcttatgatac
cagcaatgtggtattcggcggggggaccaagctgaccgtcctaggttctagaggtggtggtagcggcggcggcggctctggtggtggtgg
atccctcgagatggccgaggtgcagctggtggagtctgggggaggcttggtacagcctggagggtccctgagactctcctgtgtgcagcctctgga
ttcaccttcagtagttatgaaatgaactgggtccagggaaggggctggagtgggtttcatacattagtagtagtggtagtaccatat
actacgcagactctgtgaagggccgattcaccatctccagagacaacgccaagaactcactgtatctgcaaatgaacagcctgagagccgagga
cacggctgtttattactgtgxaxgxtgggaxtaxggtatggaxgtxggggxxaagggaxxaxgggaccacggtcaccgtctagtggccaggcc
ggccagcaccatcaccatcaccatggcgcatacccgtacgacgttccggactacgcttct [SEQ ID NO: 843]
Amino Acid Sequence
NFMLTQPHSVSESPGKTVTISCTRSSGSIASNFVQWYQQRPGSAPTPMIYEDNNRPPGVPDRF
SASVDSSSNSASLTISGLKTEDEADYYCQSYDTSNVVFGGGTKLTVLGSRGGGGSGGGGSG
GGGSLEMAEVQLVESGGGLVQPGGSLRLSCAASGFTFSSYEMNWVRQAPGKGLEWVSYIS
SSGSTIYYADSVKGRFTISRDNAKNSLYLQMNSLRAEDTAVYYCARWDYGMDVWGQGTT
VTVSSTSGQAGQHHHHHHGAYPYDVPDYAS [SEQ ID NO: 844]

TABLE 203
ET200-101

DNA Sequence
caggctgtgctgactcagccaccctcagcgtctggggcccccgggcagagggtcaccgtcttcttgttctggaagcaactccaacatcggaagta
actacgttaactggtaccagcagttcccaggaacggcccccaaactcctcatgtatagtagtagtcagcggccctcaggggtccctgaccgattct
ctggctccaagtctggcacctcagcctccctggccatcagtgggctccactctgaggatgaggctgattattactgtgctacatgggatgacagcc
tgaatgcttgggtgttcggcggagggaccaagctgaccgtcctaggttctagaggtggtggtggtagcggcggcggcggctctggtggtggtg
gatccctcgagatggccgaggtccagctggtgcagtctggggctgaggtgaggaagcctggggcctcagtgaaggtttcctgcaagacttctgg
atacaccttcacttggtatgetatacattgggtgcgccaggcccceggacaaaggettgagtggatgggatggatcaacgctggcagtggaaaca
caaaatattcacagaaatttcagggcagagtcaccettaccagggacacatccgcgagcacagcgtacatggagagagcagcctgagatctga
tgacacggctgtgtattactgtgcgagacccaataactatggttcgggtggggatgtttttgatatctggggccaagggacaatggtcaccgtactt
caactagtggccaggccggccagcaccatcaccatcaccatggcgcatacccgtacgacgttccggactacgcttct [SEQ ID NO: 845]
Amino Acid Sequence
QAVLTQPPSASGAPGQRVTVSCSGSNSNIGSNYVNWYQQFPGTAPKLLMYSSSQRPSGVPD
RFSGSKSGTSASLAISGLHSEDEADYYCATWDDSLNAWVFGGGTKLTVLGSRGGGGSGGG
GSGGGGSLEMAEVQLVQSGAEVRKPGASVKVSCKTSGYTFTWYAIHWVRQAPGQRLEWM
GWINAGSGNTKYSQKFQGRVTLTRDTSASTAYMELSSLRSDDTAVYYCARPNNYGSGGDV
FDIWGQGTMVTVSSTSGQAGQHHHHHHGAYPYDVPDYAS [SEQ ID NO: 846]

TABLE 204
ET200-102

DNA Sequence
cagtctgtcgtgacgcagccgccctcagtgtctgcggccccaggacagaaggtcaccatctcctgctctggaagcagctccaacattgggaata
attatgtatcctggtaccagcagctcccaggaacagcccccaaactcctcatttatgacaataataagcgaccctcagggattcctgaccgattctct
ggctccaagtctggcacgtcagccaccctgggcatcaccggactccagactggggacgaggccgattattactgcggaacatgggatagcagc
ctgagtgcttatgtcttcggaactgggaccaaggtcaccgtcctaggttctagaggtggtggtggtagcggcggcggcggctctggtggtggtgg
atccctcgagatggcccaggtccagctggtgcagtctggggctgaggtgaagaagcctggggcctcagtgaaagtttcctgcaaggcttctgga
tacaccttcacgaactatgctctgcattggggcgccaggcccccggacaagggcttgagtggatggcatggatcaacggtggcaatggtaaca
caaaatattcacagaacttccagggcagagtcaccattaccagggacacatccgcgagcacagcctatatggagctgagcagcctgagatctga
agacacggctgtgtattactgtgcgaaaccggaggaaacagctggaacaatccactttgactactggggccagggaaccccggtcaccgtctcc
tcaactagtggccaggccggccagcaccatcaccatcaccatggcgcatacccgtacgacgttccggactacgcttct [SEQ ID NO: 847]
Amino Acid Sequence
QSVVTQPPSVSAAPGQKVTISCSGSSSNIGNNYVSWYQQLPGTAPKLLIYDNNKRPSGIPDRF
SGSKSGTSATLGITGLQTGDEADYYCGTWDSSLSAYVFGTGTKVTVLGSRGGGGSGGGGS
GGGGSLEMAQVQLVQSGAEVKKPGASVKVSCKASGYTFTNYALHWVRQAPGQGLEWMA
WINGGNGNTKYSQNFQGRVTITRDTSASTAYMELSSLRSEDTAVYYCAKPEETAGTIHFDY
WGQGTPVTVSSTSGQAGQHHHHHHGAYPYDVPDYAS [SEQ ID NO: 848]

TABLE 205
ET200-10

DNA Sequence
caggctgtgctgactcagccccactctgtgtcggagtctccggggaagacggtaaccatctcctgcacccgcagcagtggcagcattgccagca
actatgtgcagtggtaccagcagcgcccgggcagtgcccccaccactgtgatctatgaggataaccaaagaccctctggggtccctgatcggttc
tctggctccatcgacagctcctccaactctgcctccctcaccatctctggactgaagactgaggacgaggctgactactactgtcagtcttatgatag
caccatcacggtgttcggcggagggaccaagctgaccgtcctaggttctagaggtggtggtggtagcggcggcggcggctctggtggtggtgg
atccctcgagatggcccaggtccagctggtacagtctggggctgaggtgaagaagcctgggtcctcggtgaaggtctcctgcaaggcttctgga
ggcaccttcagcagctatgctatcagctgggtgcgacaggcccctggacaagggcttgagtggatgggagggatcatccctatctttggtacagc
aaactacgcacagaagttccagggcagagtcacgattaccgcggacgaatccacgagcacagcctacatggagctgagcagcctgagatctg
aggacacggccgtgtattactgtgcgggggagggttactatgatagtagtggttattccaacggtgatgcttttgatatctggggccaagggacaat
ggtcaccgtctcttcaactagtggccaggccggccagcaccatcaccatcaccatggcgcatacccgtacgacgttccggactacgcttct [SEQ ID
NO: 849]
Amino Acid Sequence
QAVLTQPHSVSESPGKTVTISCTRSSGSIASNYVQWYQQRPGSAPTTVIYEDNQRPSGVPDRF
SGSIDSSSNSASLTISGLKTEDEADYYCQSYDSTITVFGGGTKLTVLGSRGGGGSGGGGSGG
GGSLEMAQVQLVQSGAEVKKPGSSVKVSCKASGGTFSSYAISWVRQAPGQGLEWMGGIIPI
FGTANYAQKFQGRVTITADESTSTAYMELSSLRSEDTAVYYCAGEGYYDSSGYSNGDAFDI
WGQGTMVTVSSTSGQAGQHHHHHHGAYPYDVPDYAS [SEQ ID NO: 850]

TABLE 206
ET200-104

DNA Sequence
aattttatgctgactcagccccactctgtgtcggagtctccggggaagacggtaaccatctcctgcacccgcagcagtggcagcattgccagcaa
ctatgtgcagtggtaccagcagcgcccgggcagtgcccccaccactgtgatctatgaggataaccaaagaccctctggggtccctgatcggttct
ctggctccatcgacagctcctccaactctgcctccctcaccatctctggactgaagactgaggacgaggctgactactactgtcagtcttatgatag
cagcaatgtggtattcggcggagggaccaaggtcaccgtcctaggttctagaggtggtggtggtagcggcggcggcggctctggtggtggtgg
atccctcgagatggccgaggtgcagctggtggagtctgggggaggcttggtacagcctggagggtccctgagactctcctgtgcagcctctgga
ttcaccttcagtagttatgaaatgaactgggtccgccaggctccagggaaggggctggagtgggtttcatacattagtagtagtggtagtaccatat
actacgcagactctgtgaagggccgattcaccatctccagagacaacgccaagaactcactgtatctgcaaatgaacagcctgagagccgagga
cacggctgtttattactgtgcacgctgggactacggtatggacgtctggggccaagggaccacggtcaccgtctcctcaactagtggccaggcc
ggccagcaccatcaccatcaccatggcgcatacccgtacgacgttccggactacgcttct [SEQ ID NO: 851]
Amino Acid Sequence
NFMLTQPHSVSESPGKTVTISCTRSSGSIASNYVQWYQQRPGSAPTTVIYEDNQRPSGVPDRF
SGSIDSSSNSASLTISGLKTEDEADYYCQSYDSSNVVFGGGTKVTVLGSRGGGGSGGGGSGG
GGSLEMAEVQLVESGGGLVQPGGSLRLSCAASGFTFSSYEMNWVRQAPGKGLEWVSYISSS
GSTIYYADSVKGRFTISRDNAKNSLYLQMNSLRAEDTAVYYCARWDYGMDVWGQGTTVT
VSSTSGQAGQHHHHHHGAYPYDVPDYAS [SEQ ID NO: 852]

TABLE 207
ET200-105

DNA Sequence
tcctatgtgctgactcagccaccctcagtgtccgtgtccccaggacagacagccagcatcacctgctctggagatagattgacgaataaatatgttt
cctggtatcaacagaagccaggccagtcccctgtgttggtcatctatgaggatgccaagcggccctcagggatccctgcgcgattctctggctcc
aactctgggaacacagccactctgaccatcagcgggacccaggctatggatgagtctgaatattactgtcaggcgtgggacagcagtgtggtgg
tttttggcggagggaccaagctgaccgtcctaggttctagaggtggtggtggtagcggcggcggcggctctggtggtggtggatccctcgagat
ggccgaggtgcagctggtggagtctgggggaggcttggtacagcctggcaggtccctgagactctcctgtgcagcctctggatttacctttgatg
attatgccatgcactgggtccggcaagctccagggaagggcctggagtgggtctcaggtattagttggaatagtggtagtataggctatgcggac
tctgtgaagggccgattcaccatctccagagacaacgccaagaactccctgtatctgcaaatgaacagtctgagagatgaggacacggccttgta
ttactgtgcaaaagaccgaggggggggagttatcgttaaggatgcttttgatatctggggccaagggacaatggtcaccgtctcttcaactagtggcc
aggccggccagcaccatcaccatcaccatggcgcatacccgtacgacgttccggactacgcttct [SEQ ID NO: 853]
Amino Acid Sequence
SYVLTQPPSVSVSPGQTASITCSGDRLTNKYVSWYQQKPGQSPVLVIYEDAKRPSGIPARFSG
SNSGNTATLTISGTQAMDESEYYCQAWDSSVVVFGGGTKLTVLGSRGGGGSGGGGSGGGG
SLEMAEVQLVESGGGLVQPGRSLRLSCAASGFTFDDYAMHWVRQAPGKGLEWVSGISWN
SGSIGYADSVKGRFTISRDNAKNSLYLQMNSLRDEDTALYYCAKDRGGGVIVKDAFDIWG
QGTMVTVSSTSGQAGQHHHHHHGAYPYDVPDYAS [SEQ ID NO: 854]

TABLE 208
ET200-106

DNA Sequence
tcctatgagctgactcagccacccgcagcgtctgggacccccggacagagagtcaccatctcttgttctgggggcgtctccaacatcgggagtg
gtgctctaaattggtaccagcaactcccaggaacggcccccaaactcctcatctatagttacaatcagcggccctcaggggtctctgaccgattctc
tggctccaggtctgccacctcagcctccctggccatcagtgggctccagtctgaggatgaggctgattattactgtgcaacctgggatgatagtgt
gaatggttgggtgttcggcggagggaccaagctgaccgtcctaggttctagaggtggtggtggtagcggcggcggcggctctggtggtggtgg
atccctcgagatggccgaggtgcagctggtggagtctggagctgaggtgaagaagcctggggattcagtgaaggtctcctgcaagccttctggtt
acaattttctcaactatggtatcaactgggtgcgacaggcccctggacaagggcttgagtggatgggatggattagcacttacaccggtaacaca
aactatgcacagaagctgcagggcagagtcaccttcaccacagacacatccacgagcacagcctacatggagatgaggagcctgagatctgacg
acacggccgtgtattactgtgcgcgccagcagggtggtggttggtacgatgtttggggtcaaggtactctggtcaccgtctcctcaactagtggcc
aggccggccagcaccatcaccatcaccatggcgcatacccgtacgacgttccggactacgcttct [SEQ ID NO: 855]
Amino Acid Sequence
SYELTQPPAASGTPGQRVTISCSGGVSNIGSGALNWYQQLPGTAPKLLIYSYNQRPSGVSDR
FSGSRSATSASLAISGLQSEDEADYYCATWDDSVNGWVFGGGTKLTVLGSRGGGGSGGGG
SGGGGSLEMAEVQLVESGAEVKKPGDSVKVSCKPSGYNFLNYGINWVRQAPGQGLEWMG
WISTYTGNTNYAQKLQGRVTFTTDTSTSTAYMEMRSLRSDDTAVYYCARQQGGGWYDVW
GQGTLVTVSSTSGQAGQHHHHHHGAYPYDVPDYAS [SEQ ID NO: 856]

TABLE 209
ET200-107

DNA Sequence
cagtctgtcgtgacgcagccgccctcagtgtctgcggccccaggagagaaggtcaccatctcctgctctggaagcaacttcaatgttggaaataa
tgatgtatcctggtatcagcaactcccaggtgcagcccccaaactcctcatttatgacaataataagcgaccctcagggattcctgaccgattctctg
gctccaagtctggcacgtcagccaccctggacatcaccgggctccacagtgacgacgaggccgattattactgcggaacatgggatagcagcc
tgaatactgggggggtcttcggaactgggaccaaggtcaccgtcctaggttctagaggtggtggtggtagcggcggcggcggctctggtg
gtggtggatccctcgagatggccgaggtccagctggtgcagtctggagctgaggtgaagaagcctggggcctcagtgaaggtctcctgcaaggctt
ctggttacacctttaccagctatactatcagctgggtacgacaggcccctggacaagggcttgagtggatgggatggatcagcacttacaatggtc
tcacaaactatgcacagaacctccagggcagagtcaccatgactacagacacattcacgaccacagcctacatggagctgaggagcctcagatc
tgacgacacggccgtgtattactgtgtgagagaggggtcccccgactacggtgacttcgcgtcctttgactactggggccagggaaccctggtca
ccgtctcctcaactagtggccaggccggccagcaccatcaccatcaccatggcgcatacccgtacgacgttccggactacgcttct [SEQ ID NO: 857]
Amino Acid Sequence
QSVVTQPPSVSAAPGEKVTISCSGSNFNVGNNDVSWYQQLPGAAPKLLIYDNNKRPSGIPDR
FSGSKSGTSATLDITGLHSDDEADYYCGTWDSSLNTGGVFGTGTKVTVLGSRGGGGSGGG
GSGGGGSLEMAEVQLVQSGAEVKKPGASVKVSCKASGYTFTSYTISWVRQAPGQGLEWM
GWISTYNGLTNYAQNLQGRVTMTTDTFTTTAYMELRSLRSDDTAVYYCVREGSPDYGDFA
SFDYWGQGTLVTVSSTSGQAGQHHHHHHGAYPYDVPDYAS [SEQ ID NO: 858]

TABLE 210
ET200-108

DNA Sequence
cagtctgtgttgacgcagccgccctcagtgtctgcgcccccgggacagaaggtcaccatctcctgctctggaagcagctccaacattgggaataa
ttatgtatcctggtaccagcagttcccaggaacagcccccaaactcctcatttatgacaataataagcgaccctcagggatttctgaccgattctctg
gctccaagtctggcacgtcagccaccctgggcatcgccggactccagactggggacgaggccgattattactgcggaacatgggataccagcc
tgagtggtttttatgtcttcggaagtgggaccaaggtcaccgtcctaggttctagaggtggtggtggtagcggcggcggcggctctggtggtggtg
gatccctcgagatggccgaggtccagctggtacagtctggagctgaggtgaagaagcctggggcctcagtgaaggtctcctgcaaggcttctgg
ttacacctttaccagctatactatcagctgggtacgacaggcccctggacaagggcttgagtggatgggatggatcagcacttacaatggtctcac
aaactatgcacagaacctccagggcagagtcaccatgactacagacacattcacgaccacagcctacatggagctgaggagcctcagatctga
cgacacggccgtgtattactgtgtgagagaggggtcccccgactacggtgacttcgcgtcctttgactactggggccagggaaccctggtcacc
gtctcctcaactagtggccaggccggccagcaccatcaccatcaccatggcgcatacccgtacgacgttccggactacgcttct [SEQ ID NO: 859]
Amino Acid Sequence
QSVLTQPPSVSAPPGQKVTISCSGSSSNIGNNYVSWYQQFPGTAPKLLIYDNNKRPSGISDRF
SGSKSGTSATLGIAGLQTGDEADYYCGTWDTSLSGFYVFGSGTKVTVLGSRGGGGSGGGGS
GGGGSLEMAEVQLVQSGAEVKKPGASVKVSCKASGYTFTSYTISWVRQAPGQGLEWMGW
ISTYNGLTNYAQNLQGRVTMTTDTFTTTAYMELRSLRSDDTAVYYCVREGSPDYGDFASFD
YWGQGTLVTVSSTSGQAGQHHHHHHGAYPYDVPDYAS [SEQ ID NO: 860]

TABLE 211
ET200-109

DNA Sequence
ctgcctgtgctgactcagccaccctcagcgtctgcgacccccgggcagagggtcaccatctcttgttctggaaccacctccaacatcggaagtaa
tactgtacactggtaccagcagctcccagggacggcccccaaactcctcatctataataataatcagcggccctcaggggtccctgaccgattctc
tggctccaagtctggcacctcagcctccctggccatcagtgggctccggtccgaggatgaggctacatattcctgtgcaacatgggatgacagcc
tgagtggtgtggtcttcggcggagggaccaagctgaccgtcctaggttctagaggtggtggtggtagcggcggcggcggctctggtggtggtg
gatccctcgagatggccgaggtccagctggtgcagtctggggctgaggtgaagaagcctgggtcctcggtgaaggtctcctgcaaggcttctgg
aggcaccttcagcagctatgctatcagctgggtgcgacaggcccctggacaagggcttgagtggatgggagggatcatccctatctttggtacag
caaactacgcacagaagttccagggcagagtcacgattaccgcggacgaatccacgagcacagcctacatggagctgagcagcctgagatct
gaggacacggccgtgtattactgtgcgagagatcccgcctacggtgactacgagtatgatgcttttgatatctggggccaagggacaatggtcac
cgtctcttcaactagtggccaggccggccagcaccatcaccatcaccatggcgcatacccgtacgacgttccggactacgcttct[SEQ ID NO: 861]
Amino Acid Sequence
LPVLTQPPSASATPGQRVTISCSGTTSNIGSNTVHWYQQLPGTAPKLLIYNNNQRPSGVPDRF
SGSKSGTSASLAISGLRSEDEATYSCATWDDSLSGVVFGGGTKLTVLGSRGGGGSGGGGSG
GGGSLEMAEVQLVQSGAEVKKPGSSVKVSCKASGGTFSSYAISWVRQAPGQGLEWMGGII
PIFGTANYAQKFQGRVTITADESTSTAYMELSSLRSEDTAVYYCARDPAYGDYEYDAFDIW
GQGTMVTVSSTSGQAGQHHHHHHGAYPYDVPDYAS [SEQ ID NO: 862]

TABLE 212
ET200-110

DNA Sequence
cagtctgtgttgacgcagccgccctcagcgtctgggacccccgggcagagggtcaccatctcttgttctggaagcagctccaacatcggaactaa
tggtgtaaactggttccagcagttcccaggaacggcccccaaactcctcatctatactaatgatcagcggccctcaggggtccctgaccgattctct
ggctccaagtctggcacctcagcctccctggccatcagtgggctccagtctgcggatgaggctgattattactgtgcagtgtgggaccacagcct
gaatggtccggtgttcggcggagggaccaagctgaccgtcctaggttctagaggtggtggtggtagcggcggcggcggctctggtggtggtgg
atccctcgagatggcccaggtgcagctggtgcagtctggggctgaggtgaagaagcctgggtcctcggtgaaggtctcctgcaaggcttctgga
ggcaccttcagcagctatgctatcagctgggtgcgacaggcccctggacaagggcttgagtggatgggagggatcatccctatctttggtacagc
aaactacgcacagaagttccagggcagagtcacgattaccgcggacgaatccacgagcacagcctacatggagctgagcagcctgagatctg
aggacacggccgtgtattactgtgcgagaggggccggttttgatgcttttgatatctggggccaagggacaatggtcaccgtctcttcaactagtg
gccaggccggccagcaccatcaccatcaccatggcgcatacccgtacgacgttccggactacgcttct [SEQ ID NO: 863]
Amino Acid Sequence
QSVLTQPPSASGTPGQRVTISCSGSSSNIGTNGVNWFQQFPGTAPKLLIYTNDQRPSGVPDRF
SGSKSGTSASLAISGLQSADEADYYCAVWDHSLNGPVFGGGTKLTVLGSRGGGGSGGGGS
GGGGSLEMAQVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARGAGFDAFDIWGQGT
MVTVSSTSGQAGQHHHHHHGAYPYDVPDYAS [SEQ ID NO: 864]

TABLE 213
ET200-111

DNA Sequence
caggctgtgctgactcagccaccctcagcgtctgggacccccgggcagagggtcaccatctcttgttctggaagcagctccaacatcggaagta
atactgtaaactggtaccagcagctcccaggaacggcccccaaactcctcatctatagtaataatcagcggccctcaggggtccctgaccgattct
ctggctccaagtctggcacctcagcctccctggccatcagtgggctccagtctgaggatgagactgattattactgtgcagcatgggatgacagcc
tgaatggttatgtcttcggaactgggaccaaggtcaccgtcctaggttctagaggtggtggtggtagcggcggcggcggctctggtggtggtgga
tccctcgagatggcccaggtgcagctacagcagtggggcgcaggactgttgaagccttcggagaccctgtccctcacctgcgctgtctatggtg
ggtccttcagtggttactactggagctggatccgccagcccccagggaaggggctggagtggattggggaaatcaatcatagtggaagcacca
actacaacccgtccctcaagagtcgagtcaccatatcagtagacacgtccaagaaccagttctccctgaagctgagctctgtgaccgccgcggac
acggctgtgtattactgtgcgagagaggggctagatgcttttgatatctggggccaagggacaatggtcaccgtctcttcaactagtggccaggcc
ggccagcaccatcaccatcaccatggcgcatacccgtacgacgttccggactacgcttct [SEQ ID NO: 865]
Amino Acid Sequence
QAVLTQPPSASGTPGQRVTISCSGSSSNIGSNTVNWYQQLPGTAPKLLIYSNNQRSGVPDRFP
SGSKSGTSASLAISGLQSEDETDYYCAAWDDSLNGYVFGTGTKVTVLGSRGGGGSGGGGS
GGGGSLEMAQVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCAREGLDAFDIWGQGTMV
TVSSTSGQAGQHHHHHHGAYPYDVPDYAS [SEQ ID NO: 866]

TABLE 214
ET200-112

DNA Sequence
caggctgtgctgactcagccaccctcagcgtctgggacccccgggcagagggtcaccatctcttgttctggaagcagctccaacatcggaagta
atactgtaaactggtaccagcagctcccaggaacggcccccaaactcctcatgtatagtaatgatcagcggccctcaggggtccctgaccgattct
ctggctccaagtctggcacctcagcctccctggccatcagtgggctccagtctgaggatgaggctgattattattgtgcagcatgggatgacagcc
tgaatggttatgtcttcgcagctgggacccagctcaccgttttaagttctagaggtggtggtggtagcggcggcggcggctctggtggtggtggat
ccctcgagatggcccaggtgcagctacagcagtggggcgcaggactgttgaagccttcggagaccctgtccctcacctgcgctgtctatggtgg
gtccttcagtggttactactggagctggatccgccagcccccagggaaggggctggagtggattggggaaatcaatcatagtggaagcaccaac
tacaacccgtccctcaagagtcgagtcaccatatcagtagacacgtccaagaaccagttctccctgaagctgagctctgtgaccgccgcggacac
ggctgtgtattactgtgcgagagaggggctagatgcttttgatatctggggccaagggacaatggtcaccgtctcttcaactagtggccaggccgg
ccagcaccatcaccatcaccatggcgcatacccgtacgacgttccggactacgcttct [SEQ ID NO: 867]
Amino Acid Sequence
QAVLTQPPSASGTPGQRVTISCSGSSSNIGSNTVNWYQQLPGTAPKLLMYSNDQRPSGVPDR
FSGSKSGTSASLAISGLQSEDEADYYCAAWDDSLNGYVFAAGTQLTVLSSRGGGGSGGGGS
GGGGSLEMAQVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCAREGLDAFDIWGQGTMV
TVSSTSGQAGQHHHHHHGAYPYDVPDYAS [SEQ ID NO: 868]

TABLE 215
ET200-113

DNA Sequence
cagtctgtcgtgacgcagccgccctcagtgtctgcggccccaggacagaaggtcaccatctcctgctctggaagcagctccaacattgggaata
attatgtatcctggtaccagcagctcccaggaacagcccccaaactcctcatttatgacaataataagcgaccctcagggattcctgaccgattctct
ggctccaagtctggcacgtcagccaccctgggcatcactggactccagactggggacgaggccgattattactgcggaacatgggatagcagc
ctgagtgctgcttatgtcttcggaactgggaccaaggtcaccgtcctaggttctagaggtggtggtggtagcggcggcggcggctctggtggtgg
tggatccctcgagatggcccaggtccagctggtacagtctggagctgaggtgaagaagcctggggcctcagtgaaggtctcctgcaaggcttct
ggttacagctttaccagctatactatcagctgggttcgacaggcccctggacaaggccttgagtggatgggatgggtcagcacttacaatggtctc
agaaactatgcacagaacctccagggcagagtcaccatgactacagacacactcacgaccacagcctacatggagctgaggagcctcagatct
gacgacacggccgtgtattattgtgtgagagaggggtcccccgactacggtgacttcgcggcctttgactactggggccagggcaccctggtca
ccgtctcctcaactagtggccaggccggccagcaccatcaccatcaccatggcgcatacccgtacgacgttccggactacgcttct [SEQ ID NO: 869]
Amino Acid Sequence
QSVVTQPPSVSAAPGQKVTISCSGSSSNIGNNYVSWYQQLPGTAPKLLIYDNNKRPSGIPDRF
SGSKSGTSATLGITGLQTGDEADYYCGTWDSSLSAAYVFGTGTKVTVLGSRGGGGSGGGG
SGGGGSLEMAQVQLVQSGAEVKKPGASVKVSCKASGYSFTSYTISWVRQAPGQGLEWMG
WVSTYNGLRNYAQNLQGRVTMTTDTLTTTAYMELRSLRSDDTAVYYCVREGSPDYGDFA
AFDYWGQGTLVTVSSTSGQAGQHHHHHHGAYPYDVPDYAS [SEQ ID NO: 870]

TABLE 216
ET200-114

DNA Sequence
caggctgtgctgactcagccaccctcagcgtctgagacccccgggcagagggtcaccatctcttgttctggaagcaggtccaacatcggaacta
atattgtacactggtaccagcagcgcccaggaatggcccccaaactcctcacttatggtagtcggcggccctcaggggtcccggaccgattctct
ggctccaagtttggcacctcagcctccctggccatcagtgggctccagtctgaggatgaggctgattattattgtgcagcatgggatgacagtctg
aatggtccggctttcggcggagggaccaagctgaccgtcctaggttctagaggtggtggtggtagcggcggcggcggctctggtggtggtgga
tccctcgagatggcccaggtgcagctacagcagtggggcgcaggactgttgaagccttcggagaccctgtccctcacctgcgctgtctatggtg
ggtccttcagtggttactactggagctggatccgccagcccccagggaaggggctggagtggattggggaaatcaatcatagtggaagcacca
actacaacccgtccctcaagagtcgagtcaccatatcagtagacacgtccaagaaccagttctccctgaagctgagctctgtgaccgccgcggac
acggctgtgtattactgtgcgagagacggtgggggctactttgactactggggccagggaaccctggtcaccgtctcctcaactagtggccaggc
cggccagcaccatcaccatcaccatggcgcatacccgtacgacgttccggactacgcttct [SEQ ID NO: 871]
Amino Acid Sequence
QAVLTQPPSASETPGQRVTISCSGSRSNIGTNIVHWYQQRPGMAPKLLTYGSRRPSGVPDRF
SGSKFGTSASLAISGLQSEDEADYYCAAWDDSLNGPAFGGGTKLTVLGSRGGGGSGGGGS
GGGGSLEMAQVQLQQWGAGLLKPSETLSLTCAVYGGSFSGYYWSWIRQPPGKGLEWIGEI
NHSGSTNYNPSLKSRVTISVDTSKNQFSLKLSSVTAADTAVYYCARDGGGYFDYWGQGTL
VTVSSTSGQAGQHHHHHHGAYPYDVPDYAS [SEQ ID NO: 872]

TABLE 217
ET200-115

DNA Sequence
cagtctgtgttgacgcagccgccctcagtgtctggggccccagggcagagggtcaccatctcctgcactgggagcagctccaatatcggggca
cgttatgatgtacactggtaccagcaactcccaggaacagccccccgactcctcatctctgctaactacgatcggccctcaggggtccctgaccg
attctctggctccaagtctggcacctcagcctccctggccatcactgggctccaggctgaggatgaggctgattattactgccagtcctatgacagc
agtgtgagtgcttgggtgttcggcggagggaccaaggtcaccgtcctaggttctagaggtggtggtggtagcggcggcggcggctctggtggt
ggtggatccctcgagatggccgaagtgcagctggtgcagtctggggctgaagtgaaggagcctggggcctcagtgaggatctcctgccaggc
atctggatacaacttcatcagttattatatgcactgggtgcggcaggcccctgggcaaggtcttgagtggatgggcaccatcaacccaggcagtg
gtgagacagactactcacagaagttgcagggcagagtcaccatgaccagggacccgtccacgggtacattcgacatggggctgagcagcctg
acatctggggacacggccgtctattattgtgcgacaggtctcatcagaggagctagcgatgcttttaatatctggggccgggggacaatggtcacc
gtctcttcaactagtggccaggccggccagcaccatcaccatcaccatggcgcatacccgtacgacgttccggactacgcttct [SEQ ID NO: 873]
Amino Acid Sequence
QSVLTQPPSVSGAPGQRVTISCTGSSSNIGARYDVHWYQQLPGTAPRLLISANYDRPSGVPD
RFSGSKSGTSASLAITGLQAEDEADYYCQSYDSSVSAWVFGGGTKVTVLGSRGGGGSGGG
GSGGGGSLEMAEVQLVQSGAEVKEPGASVRISCQASGYNFISYYMHWVRQAPGQGLEWM
GTINPGSGETDYSQKLQGRVTMTRDPSTGTFDMGLSSLTSGDTAVYYCATGLIRGASDAFNI
WGRGTMVTVSSTSGQAGQHHHHHHGAYPYDVPDYAS [SEQ ID NO: 874]

TABLE 218
ET200-116

DNA Sequence
cagcctgtgctgactcagccaccctcagtgtccgtgtccccaggacagacggccgccatcccctgttctggagataagttgggggataaatttgct
tcctggtatcagcagaagccaggccagtcccctgtgctggtcatctatcaagatactaagcggccctcagggatccctgagcgattctctggctcc
aactctgggaacacagccactctgaccatcagcgggacccaggctatggatgaggctgactattactgtcagacgtgggccagcggcattgtgg
tgttcggcggagggaccaagctgaccgtcctaggttctagaggtggtggtggtagcggcggcggcggctctggtggtggtggatccctcgaga
tggcccaggtacagctgcagcagtcaggtccaggactggtgaagccctcgcagaccctctcactcacctgtgccatctccggggacagtgtctct
agcaacagtgctgcttggaactggatcaggcagtccccatcgagaggccttgagtggctgggaaggacatactacaggtccaagtggtataatg
attatgcagtatctgtgaaaagtcgaataaccatcaacccagacacatccaagaaccagttctccctgcagctgaactctgtgactcccgaggaca
cggctgtgtattactgtgcaagagagcgcagtggctggaagggatttgactactggggccagggaaccctggtcaccgtctcctcaactagtggc
caggccggccagcaccatcaccatcaccatggcgcatacccgtacgacgttccggactacgcttct [SEQ ID NO: 875]
Amino Acid Sequence
QPVLTQPPSVSVSPGQTAAIPCSGDKLGDKFASWYQQKPGQSPVLVIYQDTKRPSGIPERFSG
SNSGNTATLTISGTQAMDEADYYCQTWASGIVVFGGGTKLTVLGSRGGGGSGGGGSGGGG
SLEMAQVQLQQSGPGLVKPSQTLSLTCAISGDSVSSNSAAWNWIRQSPSRGLEWLGRTYYR
SKWYNDYAVSVKSRITINPDTSKNQFSLQLNSVTPEDTAVYYCARERSGWKGFDYWGQGT
LVTVSSTSGQAGQHHHHHHGAYPYDVPDYAS [SEQ ID NO: 876]

TABLE 219
ET200-117

DNA Sequence
gatgttgtgatgactcagtctccaccctccctgtccgtcacccctggagagccggcctccatcacctgcaggtctagtcagagcctcctggaaaga
aatgcatacaactacttggattggtacctgcagaggccaggacagtctccacagctcctgatctacttgggttctaatcgggccgccggggtccct
gacaggttcagtggcagtggatcaggcagagattttacactgaaaatcagcagagtggagcctgaggatgttggggtttattactgcatgcaagct
ctacaagctccgttcactttcggcggagggaccaaggtggagatcaaacgttctagaggtggtggtggtagcggcggcggcggctctggtggt
ggtggatccctcgagatggccgaagtgcagctggtgcagtctgggggaggcttggtacagcctggggggtccctgagactctcctgtgcagcct
ctggattcacctttagcagctatgccatgagctgggtccgccaggctccagggaaggggctggagtgggtctcagctattagtggtagtggtggt
agcacatactacgcagactccgtgaagggccggttcaccatctccagagacaattccaagaacacgctgtatctgcaaatgaacagcctgagag
ccgaggacacggccgtatattactgtgcgaaatggggcccgtttcaggatgcttttgatatctggggccaagggacaatggtcaccgtctcttcaa
ctagtggccaggccggccagcaccatcaccatcaccatggcgcatacccgtacgacgttccggactacgcttct [SEQ ID NO: 877]
Amino Acid Sequence
DVVMTQSPPSLSVTPGEPASITCRSSQSLLERNAYNYLDWYLQRPGQSPQLLIYLGSNRAAG
VPDRFSGSGSGRDFTLKISRVEPEDVGVYYCMQALQAPFTFGGGTKVEIKRSRGGGGSGGG
GSGGGGSLEMAEVQLVQSGGGLVQPGGSLRLSCAASGFTFSSYAMSWVRQAPGKGLEWV
SAISGSGGSTYYADSVKGRFTISRDNSKNTLYLQMNSLRAEDTAVYYCAKWGPFQDAFDIW
GQGTMVTVSSTSGQAGQHHHHHHGAYPYDVPDYAS [SEQ ID NO: 878]

TABLE 220
ET200-118

DNA Sequence
caggctgtgctgactcagcctgcctccgtgtctgggtctcctggacagtcgatcaccatctcctgcactggaaccagcagtgacgttggtggttata
actatgtctcctggtaccaacagcacccgggcaaagcccccaaactcatgatttatgaggtcagtaatcggccctcaggggtttctaatcgcttctct
ggctccaagtctggcaacacggcctccctgaccatctctgggctccaggctgaggacgaggctgattattactgcagctcatatacaagcagcag
caccccttatgtcttcggagcagggaccaaggtcaccgtcctaggttctagaggtggtggtggtagcggcggcggcggctctggtggtggtgga
tccctcgagatggccgaggtgcagctggtggagtctgggggaggcttggtacagcctggcaggtccctgagactctcctgtgcagcctctggatt
cacctttgatgattatgccatgcactgggtccggcaagctccagggaagggcctggagtgggtctcaggtattagttggaatagtggtagcatagg
ctatgcggactctgtgaagggccgattcaccatctccagagacaacgccaagaactccctgtatctgcaaatgaacagtctgagagctgaggaca
cggccttgtattactgtgcaaaagccaggtggacagcagtggcatcagaccaccactttgactactggggccagggaacgctggtcaccgtctc
ctcaactagtggccaggccggccagcaccatcaccatcaccatggcgcatacccgtacgacgttccggactacgcttct [SEQ ID NO: 879]
Amino Acid Sequence
QAVLTQPASVSGSPGQSITISCTGTSSDVGGYNYVSWYQQHPGKAPKLMIYEVSNRPSGVSN
RFSGSKSGNTASLTISGLQAEDEADYYCSSYTSSSTPYVFGAGTKVTVLGSRGGGGSGGGGS
GGGGSLEMAEVQLVESGGGLVQPGRSLRLSCAASGFTFDDYAMHWVRQAPGKGLEWVSG
ISWNSGSIGYADSVKGRFTISRDNAKNSLYLQMNSLRAEDTALYYCAKARWTAVASDHHF
DYWGQGTLVTVSSTSGQAGQHHHHHHGAYPYDVPDYAS [SEQ ID NO: 880]

TABLE 221
ET200-119

DNA Sequence
caggctgtgcttactcagccaccctcagcgtctgggacccccgggcagagggtcaccatctcttgttctggaagcagctccaacatcggaagtaa
tactgtaaactggtaccagcagctcccaggaacggcccccaaactcctcatctatagtaataatcagcggccctcaggggtccctgaccgattctc
tggctccaagtctggcacctcagcctccctggccatcagtgggctccagtctgaggatgaggctgattattactgtgcagcatgggatgacagcct
gaatggttatgtcttcggaactgggaccaagctgaccgtcctaggttctagaggtggtggtggtagcggcggcggcggctctggtggtggtggat
ccctcgagatggccgaggtgcagctggtgcagtctggggctgaggtgaagaagcctgggtcctcggtgaaggtctcctgcaaggcttctggag
gcaccttcagcagctatgctatcagctgggtgcgacaggcccctggacaagggcttgagtggatgggagggatcatccctatctttggtacagca
aactacgcacagaagttccagggcagagtcacgattaccgcggacgaatccacgagcacagcctacatggagctgagcagcctgagatctga
ggacacggccgtgtattactgtgcgagagattgggactacatggacgtctggggcaaagggaccacggtcaccgtctcctcaactagtggccag
gccggccagcaccatcaccatcaccatggcgcatacccgtacgacgttccggactacgcttct [SEQ ID NO: 881]
Amino Acid Sequence
QAVLTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYVFGTGTKLTVLGSRGGGGSGGGGS
GGGGSLEMAEVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARDWDYMDVWGKGTT
VTVSSTSGQAGQHHHHHHGAYPYDVPDYAS [SEQ ID NO: 882]

TABLE 222
ET200-120

DNA Sequence
tcctatgagctgactcagccaccctcagcgtctgggacccccgggcagagggtcaccatctcttgttctggaagcagctccaacatcggaagtaa
tactgtaaactggtaccagcagctcccaggaacggcccccaaactcctcatctatagtaataatcagcggccctcaggggtccctgaccgattctc
tggctccaagtctggcacctcagcctccctggccatcagtgggctccagtctgaggatgaggctgattattactgtgcagcatgggatgacagcct
gaatggttatgtcttcggaactgggaccaaggtcaccgtcctaggttctagaggtggtggtggtagcggcggcggcggctctggtggtggtggat
ccctcgagatggccgaggtgcagctggtggagtctggagctgaggtgaagaagcctggggcctcagtgaaggtctcctgcaaggcttctggtta
cacctttaccagctatggtatcagctgggtgcgacaggcccctggacaagggcttgagtggatgggatggatcagcgcttacaatggtaacacaa
actatgcacagaagctccagggcagagtcaccatgaccacagacacatccacgagcacagcctacatggagctgaggagcctgagatctgac
gacacggccgtgtattactgtgcgagagacctatctcggggagctaacccgcattactactactactacggtatggacgtctggggccaaggac
cacggtcaccgtctcctcaactagtggccaggccggccagcaccatcaccatcaccatggcgcatacccgtacgacgttccggactacgcttct
[SEQ ID NO: 883]
Amino Acid Sequence
SYELTQPPSASGTPGQRVTISCSGSSSNIGSNTVNWYQQLPGTAPKLLIYSNNQRPSGVPDRF
SGSKSGTSASLAISGLQSEDEADYYCAAWDDSLNGYVFGTGTKVTVLGSRGGGGSGGGGS
GGGGSLEMAEVQLVESGAEVKKPGASVKVSCKASGYTFTSYGISWVRQAPGQGLEWMGW
ISAYNGNTNYAQKLQGRVTMTTDTSTSTAYMELRSLRSDDTAVYYCARDLSRGANPHYYY
YYGMDVWGQGTTVTVSSTSGQAGQHHHHHHGAYPYDVPDYAS [SEQ ID NO: 884]

TABLE 223
ET200-121

DNA Sequence
cagtctgtgttgacgcagccgccctcagtgtctggggccccagggcagagggtcaccgtctcctgcactgggagcagatccaacatcggggca
ggatatgatgtacactggtaccagcaacttccaggaacagcccccaaactcctcatctatggaaatagtaatcggcctccaggggtccctgaccg
attctctgggtctaagtctggcacctcagcctccctggtcatcactgggctccaggctgaggatgccgctgattattactgccagtcctatgacaaca
ctgtgcgtgaatcaccttatgtcttcggaactgggaccaaggtcaccgtcctaggttctagaggtggtggtggtagcggcggcggcggctctggt
ggtggtggatccctcgagatggccgaggtccagctggtacagtctggggctgaggtgaagaagcctggggcctcagtgaaggtctcctgcaag
gtttccggatacaccctcactgaattatccatgcactgggtgcgacaggctcctggaaaagggcttgagtggatgggaggttttgatcctgaagat
ggtgaaacaatctacgcacagaagttccagggcagagtcaccatgaccgaggacacatctacagacacagcctacatggagctgagcagcctg
agatctgaggacacggccgtgtattactgtgcaacagagagtaatttagtgtcccggcactactactactacggtatggacgtctggggccaaggg
accacggtcaccgtctcctcaactagtggccaggccggccagcaccatcaccatcaccatggcgcatacccgtacgacgttccggactacgcttct
[SEQ ID NO: 885]
Amino Acid Sequence
QSVLTQPPSVSGAPGQRVTVSCTGSRSNIGAGYDVHWYQQLPGTAPKLLIYGNSNRPPGVP
DRFSGSKSGTSASLVITGLQAEDAADYYCQSYDNTVRESPYVFGTGTKVTVLGSRGGGGSG
GGGSGGGGSLEMAEVQLVQSGAEVKKPGASVKVSCKVSGYTLTELSMHWVRQAPGKGLE
WMGGFDPEDGETIYAQKFQGRVTMTEDTSTDTAYMELSSLRSEDTAVYYCATESNLVSRH
YYYYGMDVWGQGTTVTVSSTSGQAGQHHHHHHGAYPYDVPDYAS [SEQ ID NO: 886]

TABLE 224
ET200-122

DNA Sequence
ctgcctgtgctgactcagccaccctcagcgtctgggacccccgggcagagggtcaccatctcttgttctggaaccagctccaacatcggaagtaa
ttctgtagactggtaccagcagctcccaggaacggcccccaaactcctcatctatagtaataatcagcggccctcaggggtccctgaccgaatctc
tggctccaagtctggcacctcagcctccctggccatcagtgggctccagtctgaggatgaggctgattattactgtgcagcatgggatgacagcct
gaatggttatgtcttcggaactgggaccaaggtcaccgtcctaggttctagaggtggtggtggtagcggcggcggcggctctggtggtggtggat
ccctcgagatggccgaagtgcagctggtgcagtctggggctgaggtgaagaagcctggggcctcagtgaaggtctcctgcaaggcttctggat
acaccttcaccggctactatatgcactgggtgcgacaggcccctggacaagggcttgagtggatgggatggatcaaccctaacagtggtggcac
aaactatgcacagaagtttcagggcagggtcaccatgaccagggacacgtccatcagcacagcctacatggagctgagcaggctgagatctga
cgacacggccgtgtattactgtgcgagagattacggatactatggttcggggagttattcgagcggccccctttactactactacggtatggacg
tctggggccaagggaccacggtcaccgtctcctcaactagtggccaggccggccagcaccatcaccatcaccatggcgcata
cccgtacgacgttccggactacgcttct [SEQ ID NO: 887]
Amino Acid Sequence
LPVLTQPPSASGTPGQRVTISCSGTSSNIGSNSVDWYQQLPGTAPKLLIYSNNQRPSGVPDRIS
GSKSGTSASLAISGLQSEDEADYYCAAWDDSLNGYVFGTGTKVTVLGSRGGGGSGGGGSG
GGGSLEMAEVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCARDYGYYGSGSYS
SGPLYYYYGMDVWGQGTTVTVSSTSGQAGQHHHHHHGAYPYDVPDYAS [SEQ ID NO: 888]

TABLE 225
ET200-123

DNA Sequence
caggctgtgctgactcagccaccctcagcgtctgggacccccgggcagagggtcaccatctcttgttctggaagcagctccaacatcggaagta
atactgtaaactggtaccagcagctcccaggaacggcccccaaactcctcatgtataataatgatcagcggccctcaggggtccctgaccgattct
ctggctccaagtctggcacctcagcctccctggccatcagtgggctccagtctgaggatgaggctgattattactgtgcagcatgggatgacagc
ctcaatggttatgtcttcggacctgggaccaaggtcaccgtcctaggttctagaggtggtggtggtagcggcggcggcggctctggtggtggtgg
atccctcgagatggcccaggtgcagctggtggagtctggagctgaggtgaagaagcctggggcctcagtgaaggtctcctgcaaggcttctggt
tacacctttaccagctatggtatcagctgggtgcgacaggcccctggacaagggcttgagtggatgggatggatcagcgcttacaatggtaacac
aaactatgcacagaagctccagggcagagtcaccatgaccacagacacatccacgagcacagcctacatggagctgaggagcctgagatctg
acgacacggccgtgtattactgtgcgagagacctatctcggggagctaacccgcattactactactactacggtatggacgtctggggccaaggg
accacggtcaccgtctcctcaactagtggccaggccggccagcaccatcaccatcaccatggcgcatacccgtacgacgttccggactacgcttct
[SEQ ID NO: 889]
Amino Acid Sequence
QAVLTQPPSASGTPGQRVTISCSGSSSNIGSNTVNWYQQLPGTAPKLLMYNNDQRPSGVPD
RFSGSKSGTSASLAISGLQSEDEADYYCAAWDDSLNGYVFGPGTKVTVLGSRGGGGSGGG
GSGGGGSLEMAQVQLVESGAEVKKPGASVKVSCKASGYTFTSYGISWVRQAPGQGLEWM
GWISAYNGNTNYAQKLQGRVTMTTDTSTSTAYMELRSLRSDDTAVYYCARDLSRGANPH
YYYYYGMDVWGQGTTVTVSSTSGQAGQHHHHHHGAYPYDVPDYAS [SEQ ID NO: 890]

TABLE 226
ET200-125

DNA Sequence
aattttatgctgactcagccccacgctgtgtcggagtctccggggaagacggtaaccatctcctgcacccgcagcagtggcagtattgccagcaa
ctatgtgcagtggtaccagcagcgcccgggcagttccccccgcactgtgatttatgaggataatcaaagaccctctggggtccctggtcggttctc
tggctccatcgacagctcctccaactctgcctccctcaccatctctggactgaagactgaggacgaggctgactactactgtcagtcttatgattcca
ccagtgtgcttttcggcggagggaccaagctgaccgtcctaggttctagaggtggtggtggtagcggcggcggcggctctggtggtggtggatc
cctcgagatggccgaggtccagctggtgcagtctggggctgaggtgaagaagccagggtcctcggtgaaggtctcctgcaaggcctcgggag
gcaccttcagcagcaattctctcagctgggtgcgacaggcccctggacaagggcttgagtggatgggaaggatcttccctatcctgggtataaca
aactatgcacagaagttccagggcagagtcacgattaccgcggacaaatccacgagcacagcctacatggagctgagcagcctgagatctgag
gacacggccgtctattactgtgcgagaggaaactaccaatggtatgatgcttttgatatctggggccaagggacaatggtcaccgtctcttcaacta
tagtggccaggccggccagcaccatcaccatcaccatggcgcatacccgtacgacgttccggactacgcttct [SEQ ID NO: 891]
Amino Acid Sequence
NFMLTQPHAVSESPGKTVTISCTRSSGSIASNYVQWYQQRPGSSPRTVIYEDNQRPSGVPGR
FSGSIDSSSNSASLTISGLKTEDEADYYCQSYDSTSVLFGGGTKLTVLGSRGGGGSGGGGSG
GGGSLEMAEVQLVQSGAEVKKPGSSVKVSCKASGGTFSSNSLSWVRQAPGQGLEWMGRIF
PILGITNYAQKFQGRVTITADKSTSTAYMELSSLRSEDTAVYYCARGNYQWYDAFDIWGQG
TMVTVSSTSGQAGQHHHHHHGAYPYDVPDYAS [SEQ ID NO: 892]

TABLE 227
ET200-005

DNA Sequence
cagcctgtgctgactcagccaccctcagtgtcagtggtcccaggaaagacggccaggattacctgtgggggaaaaaacattggaagtaaaagtgt
gcactggtaccagcagaagccaggccaggcccctgtggtggtcatccattatgatagtgaccggccctcagggatccctgagcgattctctggc
tccaactctgggaacacggccaccctgaccatcagcagggtcgaagccggggatgaggccgactattactgtcaggtgtgggatagtagtagtg
atcatccttatgtcttcggaactgggaccaaggtcaccgtcctaggttctagaggtggtggtggtagcggcggcggcggctctggtggtggtgga
tccctcgagatggcccaggtgcagctggtgcagtctggagctgaggtgaagaagcctggggcctcagtgaaggtctcctgcaaggcttctggtt
acacctttaccaactatggtatcagctgggtgcgacaggcccctggacaagggcttgagtggatgggatggatcagcgcttacaatggtaacaca
aactatgcacataagctccagggcagagtcaccatgaccacagacacatccacgagcacagccaacatggagctgaggagcctgagacctga
cgacactgccgtgtattactgtgcgcgctcttacttcggttctcatgattactggggtcaaggtactctggtgaccgtctcctcaactagtggccagg
ccggccagcaccatcaccatcaccatggcgcatacccgtacgacgttccggactacgcttct [SEQ ID NO: 893]
Amino Acid Sequence
QPVLTQPPSVSVVPGKTARITCGGKNIGSKSVHWYQQKPGQAPVVVIHYDSDRPSGIPERFS
GSNSGNTATLTISRVEAGDEADYYCQVWDSSSDHPYVFGTGTKVTVLGSRGGGGSGGGGS
GGGGSLEMAQVQLVQSGAEVKKPGASVKVSCKASGYTFTNYGISWVRQAPGQGLEWMG
WISAYNGNTNYAHKLQGRVTMTTDTSTSTANMELRSLRPDDTAVYYCARSYFGSHDYWG
QGTLVTVSSTSGQAGQHHHHHHGAYPYDVPDYAS [SEQ ID NO: 894]

TABLE 228
ET200-124

DNA Sequence
tcctatgtgctgactcagccaccctcggtgtcagtggccccaggaaagacggccaggatttcctgtgggggaaacgacattggaagtaaaagtgt
tttctggtatcagcagaggccaggccaggcccctgtgttggtcgtctatgatgatagcgaccggccctcagggctccctgagcgattctctggctt
caactctgggaacacggccaccctgaccatcagcagggtcgaagccggggatgaggccgactattactgtcaagtgtgggatagtagtagtgat
cattatgtcttcggaactgggaccaaggtcaccgtcctaggttctagaggtggtggtggtagcggcggcggcggctctggtggtggtggatccct
cgagatggcccaggtgcagctggtggagtctgggggaggcttggtacagcctggcaggtccctgagactctcctgtgcagcctctggattcacc
tttgatgattatgccatgcactgggtccggcaagctccagggaagggcctggagtgggtctcaggtattagttggaatagtggtagcataggctat
gcggactctgtgaagggccgattcaccatctccagagacaacgccaagaactccctgtatctgcaaatgaacagtctgagagctgaggacacgg
ccttgtattactgtgcaaaagatataacctatggttcggggagttatggtgcttttgatatctggggccaagggacaatggtcaccgtctctt
caactagtggccaggccggccagcaccatcaccatcaccatggcgcatacccgtacgacgttccggactacgcttct [SEQ ID NO: 895]
Amino Acid Sequence
SYVLTQPPSVSVAPGKTARISCGGNDIGSKSVFWYQQRPGQAPVLVVYDDSDRPSGLPERFS
GFNSGNTATLTISRVEAGDEADYYCQVWDSSSDHYVFGTGTKVTVLGSRGGGGSGGGGSG
GGGSLEMAQVQLVESGGGLVQPGRSLRLSCAASGFTFDDYAMHWVRQAPGKGLEWVSGI
SWNSGSIGYADSVKGRFTISRDNAKNSLYLQMNSLRAEDTALYYCAKDITYGSGSYGAFDI
WGQGTMVTVSSTSGQAGQHHHHHHGAYPYDVPDYAS [SEQ ID NO: 896]

TABLE 229

| Anti- | | | | | | |
|---|---|---|---|---|---|---|
| body | V_{H}CDR1 | V_{H}CDR2 | V_{H}CDR3 | V_{L}CDR1 | V_{L}CDR2 | V_{L}CDR3 |
| ET200- | GGSFSGYY | INHSGST | AREGPYDGFDS | SSNIGSNT | SNN | AAWDDSLNGYV |
| 001 | [SEQ ID | [SEQ ID | [SEQ ID NO: 311] | [SEQ ID | [SEQ ID | [SEQ ID NO: 314] |
| | NO: 309] | NO: 310] | | NO: 312] | NO: 313] | |
| ET200- | GYPFNKYD | IIPIFRTT | AREWFYWDI | SGSIASNY | EDN | QSYDSSNSVV |
| 002 | [SEQ ID | [SEQ ID | [SEQ ID NO: 317] | [SEQ ID | [SEQ ID | [SEQ ID NO: 320] |
| | NO: 315] | NO: 316] | | NO: 318] | NO: 319] | |
| ET200- | GFTFSSYG | ISHDGSNK | ARSNQWSGYFSFDY | KLGTKY | EDN | QAWDSDTFVV |
| 003 | [SEQ ID | [SEQ ID | [SEQ ID NO: 323] | [SEQ ID | [SEQ ID | [SEQ ID NO: 325] |
| | NO: 321] | NO: 322] | | NO: 324] | NO: 319] | |
| ET200- | GYTFTNYG | ISAYNGNT | ARSYFGSHDY | NIGSKS | YDS | QVWDSSSDHPYV |
| 005 | [SEQ ID | [SEQ ID | [SEQ ID NO: 328] | [SEQ ID | [SEQ ID | [SEQ ID NO: 331] |
| | NO: 326] | NO: 327] | | NO: 329] | NO: 330] | |
| ET200- | GYTFTTYG | INTYNGHT | ARVIYGSGDY | NIGSKS | YDS | QVWDSSSDHPYV |
| 006 | [SEQ ID | [SEQ ID | [SEQ ID NO: 334] | [SEQ ID | [SEQ ID | [SEQ ID NO: 331] |
| | NO: 332] | NO: 333] | | NO: 329] | NO: 330] | |
| ET200- | GYSISSGYF | IYHSRST | ARGYGYFDY | NIGSKT | YDS | QVWDSSDHRV |
| 007 | [SEQ ID | [SEQ ID | [SEQ ID NO: 337] | [SEQ ID | [SEQ ID | [SEQ ID NO: 339] |
| | NO: 335] | NO: 336] | | NO: 338] | NO: 330] | |
| ET200- | GFTFGDYG | INWNGGST | ARSKYNFHVYYDY | SSDVGGYNY | DVS | SSYTSSSTSKV |
| 008 | [SEQ ID | [SEQ ID | [SEQ ID NO: 342] | [SEQ ID | [SEQ ID | [SEQ ID NO: 345] |
| | NO: 340] | NO: 341] | | NO: 343] | NO: 344] | |
| ET200- | GYTFTSYG | ISAYNGNT | ARSSGNMVSWKDM | NSNIGSNY | RNN | AAWDDSLSAYV |
| 009 | [SEQ ID | [SEQ ID | [SEQ ID NO: 347] | [SEQ ID | [SEQ ID | [SEQ ID NO: 350] |
| | NO: 346] | NO: 327] | | NO: 348] | NO: 349] | |
| ET200- | GYTFTSYG | ISAYNGNT | ARGAVAYHD | SSDVGGYNS | DVS | SSYTSSSTPLV |
| 010 | [SEQ ID | [SEQ ID | [SEQ ID NO: 351] | [SEQ ID | [SEQ ID | [SEQ ID NO: 353] |
| | NO: 346] | NO: 327] | | NO: 352] | NO: 344] | |
| ET200- | GGTLSSYA | IIPMFGTA | ARGVHYASFDH | SSNISIYD | GNN | GTWDDSLSGGV |
| 011 | [SEQ ID | [SEQ ID | [SEQ ID NO: 356] | [SEQ ID | [SEQ ID | [SEQ ID NO: 359] |
| | NO: 354] | NO: 355] | | NO: 357] | NO: 358] | |
| ET200- | GFPFNIFG | ISGYNGNT | ARGAYGGMDT | DSNIGNNY | DVK | GTWDSRLDAYV |
| 012 | [SEQ ID | [SEQ ID | [SEQ ID NO: 362] | [SEQ ID | [SEQ ID | [SEQ ID NO: 365] |
| | NO: 360] | NO: 361] | | NO: 363] | NO: 364] | |
| ET200- | GYMFTSYG | ISANNGKT | ARHIGGSYFDR | TSNIGAGYD | TNN | GTWDSSLSAVV |
| 013 | [SEQ ID | [SEQ ID | [SEQ ID NO: 368] | [SEQ ID | [SEQ ID | [SEQ ID NO: 371] |
| | NO: 366] | NO: 367] | | NO: 369] | NO: 370] | |
| ET200- | GFTFSSYA | ISGSDGST | ARSHEANLVGDW | NIGSKS | YDS | QVWDSSSDHYV |
| 014 | [SEQ ID | [SEQ ID | [SEQ ID NO: 374] | [SEQ ID | [SEQ ID | [SEQ ID NO: 375] |
| | NO: 372] | NO: 373] | | NO: 329] | NO: 330] | |
| ET200- | GYTFTSYG | ISAYNGNT | ARWGGFGAVDH | NIGSKS | YDS | QVWDSSSDVV |
| 015 | [SEQ ID | [SEQ ID | [SEQ ID NO: 376] | [SEQ ID | [SEQ ID | [SEQ ID NO: 377] |
| | NO: 346] | NO: 327] | | NO: 329] | NO: 330] | |
| ET200- | GFTFSSYS | ISSSSSYI | ARGQGYDY | SLTDYH | ATN | NSRDSGTDEVL |
| 016 | [SEQ ID | [SEQ ID | [SEQ ID NO: 380] | [SEQ ID | [SEQ ID | [SEQ ID NO: 383] |
| | NO: 378] | NO: 379] | | NO: 381] | NO: 382] | |
| ET200- | GGSFSGYY | INHSGST | ARYYPGMDM | NIGSKS | DDS | QVWDSSSDHTV |
| 017 | [SEQ ID | [SEQ ID | [SEQ ID NO: 384] | [SEQ ID | [SEQ ID | [SEQ ID NO: 386] |
| | NO: 309] | NO: 310] | | NO: 329] | NO: 385] | |
| ET200- | GYTLNELS | FDPEDGET | ARGGYGDS | SSNIGRNG | NDN | AAWDDSLHGVV |
| 018 | [SEQ ID | [SEQ ID | [SEQ ID NO: 389] | [SEQ ID | [SEQ ID | [SEQ ID NO: 392] |
| | NO: 387] | NO: 388] | | NO: 390] | NO: 391] | |
| ET200- | GGTFSSDA | IIPMFGTA | AREGYYYPSAYLGSVLNDISSVYDE | SGSIASNY | EDN | QSYDSSNSWV |
| 019 | [SEQ ID | [SEQ ID | [SEQ ID NO: 394] | [SEQ ID | [SEQ ID | [SEQ ID NO: 395] |
| | NO: 393] | NO: 355] | | NO: 318] | NO: 319] | |
| ET200- | GYTFTSYG | ISAYNGNT | ARSMTSFDY | TSNIGNND | DNN | GTWDSSVSAS |
| 020 | [SEQ ID | [SEQ ID | [SEQ ID NO: 396] | [SEQ ID | [SEQ ID | [SEQ ID NO: 399] |
| | NO: 346] | NO: 327] | | NO: 397] | NO: 398] | |
| ET200- | GYTFTSYG | ISAYNGNT | ARSVYDLDT | NSNIGNNY | DNN | GTWNTTVTPGYV |
| 021 | [SEQ ID | [SEQ ID | [SEQ ID NO: 400] | [SEQ ID | [SEQ ID | [SEQ ID NO: 402] |
| | NO: 346] | NO: 327] | | NO: 401] | NO: 398] | |
| ET200- | GFTFDDYA | ISWNSGSI | ARYRQVGSAYDS | SSNIGNNY | DNN | GTWDSSLGAPYV |
| 022 | [SEQ ID | [SEQ ID | [SEQ ID NO: 405] | [SEQ ID | [SEQ ID | [SEQ ID NO: 407] |
| | NO: 403] | NO: 404] | | NO: 406] | NO: 398] | |
| ET200- | GYTFTSYG | ISAYNGNT | ARYWGFGVSDR | NIGSKS | ADS | QVWDSSSYHNYV |
| 023 | [SEQ ID | [SEQ ID | [SEQ ID NO: 408] | [SEQ ID | [SEQ ID | [SEQ ID NO: 410] |
| | NO: 346] | NO: 327] | | NO: 329] | NO: 409] | |
| ET200- | GGTFSSYA | IIPIFGTA | ARYNYYYYDS | SGSIASNY | EDN | QSYDSSNLWV |
| 024 | [SEQ ID | [SEQ ID | [SEQ ID NO: 413] | [SEQ ID | [SEQ ID | [SEQ ID NO: 414] |
| | NO: 411] | NO: 412] | | NO: 318] | NO: 319] | |
| ET200- | GGTFSSYA | IIPIFGTA | ARYWGYDSYDE | QSISSY | AAS | QQSYSTPFT |
| 025 | [SEQ ID | [SEQ ID | [SEQ ID NO: 415] | [SEQ ID | [SEQ ID | [SEQ ID NO: 418] |
| | NO: 411] | NO: 412] | | NO: 416] | NO: 417] | |
| ET200- | GGTFSSYA | IIPIFGTA | ARNNHYYNDY | SGSIASNY | EDN | QSYDSSNWV |
| 026 | [SEQ ID | [SEQ ID | [SEQ ID NO: 349] | [SEQ ID | [SEQ ID | [SEQ ID NO: 419] |
| | NO: 411] | NO: 412] | | NO: 318] | NO: 319] | |
| ET200- | GYTFTDYY | VDPEDGET | ARYWSYSFDYLYMPEGNDW | SSNIGAGYD | GNN | QSYDSSLSDVV |
| 027 | [SEQ ID | [SEQ ID | [SEQ ID NO: 422] | [SEQ ID | [SEQ ID | [SEQ ID NO: 424] |
| | NO: 420] | NO: 421] | | NO: 423] | NO: 358] | |
| ET200- | GYNFLNYG | ISTYTGNT | ARDLYYYEGVDY | VSNIGSGA | SYN | ATWDDSVNG |
| 028 | [SEQ ID | [SEQ ID | [SEQ ID NO: 427] | [SEQ ID | [SEQ ID | [SEQ ID NO: 430] |
| | NO: 425] | NO: 426] | | NO: 428] | NO: 429] | |
| ET200- | GFTFSSYA | ISYDGSNK | ARSYFTSGFYDY | NIGSES | YDT | QVWDSSRDHVV |
| 029 | [SEQ ID | [SEQ ID | [SEQ ID NO: 432] | [SEQ ID | [SEQ ID | [SEQ ID NO: 435] |
| | NO: 372] | NO: 431] | | NO: 433] | NO: 434] | |
| ET200- | GYTLTELS | FDPEDGET | ARMSSMYYD | SSNIGAGYD | GNS | QSYDSSLSGSYV |
| 030 | [SEQ ID | [SEQ ID | [SEQ ID NO: 437] | [SEQ ID | [SEQ ID | [SEQ ID NO: 439] |
| | NO: 436] | NO: 388] | | NO: 423] | NO: 438] | |
| ET200- | GFTVSDYY | ISGSGNSI | ARSTKFDY | NIGSKS | YDS | QVWDSSSDYV |
| 031 | [SEQ ID | [SEQ ID | [SEQ ID NO: 442] | [SEQ ID | [SEQ ID | [SEQ ID NO: 443] |
| | NO: 440] | NO: 441] | | NO: 329] | NO: 330] | |
| ET200- | GYSFTNYW | IYPGDSDT | ARSTGSSHMSDE | SSNVGSYT | NNN | AAWDDRLGGYV |
| 032 | [SEQ ID | [SEQ ID | [SEQ ID NO: 446] | [SEQ ID | [SEQ ID | [SEQ ID NO: 449] |
| | NO: 444] | NO: 445] | | NO: 447] | NO: 448] | |
| ET200- | GGSFSGYY | ITHSGRS | ARSSIMSDY | SGSIASNY | EDN | QSYDSSNHWV |
| 033 | [SEQ ID | [SEQ ID | [SEQ ID NO: 451] | [SEQ ID | [SEQ ID | [SEQ ID NO: 452] |
| | NO: 309] | NO: 450] | | NO: 318] | NO: 319] | |
| ET200- | GGTFSSYA | IIPIFGTA | ARGSALDHYDR | TSNIGAGYD | NNR | GTWDGSLTGAV |
| 034 | [SEQ ID | [SEQ ID | [SEQ ID NO: 453] | [SEQ ID | [SEQ ID | [SEQ ID NO: 455] |
| | NO: 411] | NO: 412] | | NO: 369] | NO: 454] | |
| ET200- | GGTFSSYA | IIPIFGTA | ARYNYYFNDY | SGSIASNY | EDN | QSYDSTNWV |
| 035 | [SEQ ID | [SEQ ID | [SEQ ID NO: 456] | [SEQ ID | [SEQ ID | [SEQ ID NO: 457] |
| | NO: 411] | NO: 412] | | NO: 318] | NO: 319] | |
| ET200- | GYTFTSYG | ISAYNGNT | ARSMFGAHDS | NIGSKS | YDS | QVWDSSSDHPYV |
| 037 | [SEQ ID | [SEQ ID | [SEQ ID NO: 458] | [SEQ ID | [SEQ ID | [SEQ ID NO: 331] |
| | NO: 346] | NO: 327] | | NO: 329] | NO: 330] | |
| ET200- | GGTFSSYA | IIPIFGTA | ARGASFDRHDN | SSNIGAGFD | ANS | QSYDSSLSGVV |
| 038 | [SEQ ID | [SEQ ID | [SEQ ID NO: 459] | [SEQ ID | [SEQ ID | [SEQ ID NO: 462] |
| | NO: 411] | NO: 412] | | NO: 460] | NO: 461] | |
| ET200- | GGTFSSYA | IIPIFGTA | ARSNYYYNDY | SGSIASNY | EDN | QSYDSSNWV |
| 039 | [SEQ ID | [SEQ ID | [SEQ ID NO: 463] | [SEQ ID | [SEQ ID | [SEQ ID NO: 419] |
| | NO: 411] | NO: 412] | | NO: 318] | NO: 319] | |
| ET200- | GYTLTELS | FDPEDGET | ARYSGVYYD | SSNIGAGYD | GNS | ASYDSSLSGYV |
| 040 | [SEQ ID | [SEQ ID | [SEQ ID NO: 464] | [SEQ ID | [SEQ ID | [SEQ ID NO: 465] |
| | NO: 436] | NO: 388] | | NO: 423] | NO: 438] | |
| ET200- | GGTFSSYA | MNPNSGNT | ARYYSYGYD | SGSIADNF | NDD | QSYDNNNRGV |
| 041 | [SEQ ID | [SEQ ID | [SEQ ID NO: 467] | [SEQ ID | [SEQ ID | [SEQ ID NO: 470] |
| | NO: 411] | NO: 466] | | NO: 468] | NO: 469] | |
| ET200- | GDSVSTNSVA | TYYRSKWSN | ARSSSWYQIFDY | SSNIGTGYF | GNN | QSYDSSLSGYV |
| 042 | [SEQ ID | [SEQ ID | [SEQ ID NO: 473] | [SEQ ID | [SEQ ID | [SEQ ID NO: 465] |
| | NO: 471] | NO: 472] | | NO: 474] | NO: 358] | |
| ET200- | GFTFSSYA | ISGSGGST | ARSGAYWDYSVYDE | SDSIANNY | EDV | QSYHSDNRWV |
| 043 | [SEQ ID | [SEQ ID | [SEQ ID NO: 486] | [SEQ ID | [SEQ ID | [SEQ ID NO: 479] |
| | NO: 372] | NO: 475] | | NO: 477] | NO: 478] | |
| ET200- | GGSISSSNW | IYHSGSP | ARMTTHTFGYDA | KLGDKY | QDN | QAWDSSTYVA |
| 044 | [SEQ ID | [SEQ ID | [SEQ ID NO: 482] | [SEQ ID | [SEQ ID | [SEQ ID NO: 485] |
| | NO: 480] | NO: 481] | | NO: 483] | NO: 484] | |
| ET200- | GYTFTSYG | ISAYNGNT | ARGVHLDW | NIGSES | DDA | QVWDRNSAQFV |
| 045 | [SEQ ID | [SEQ ID | [SEQ ID NO: 486] | [SEQ ID | [SEQ ID | [SEQ ID NO: 488] |
| | NO: 346] | NO: 327] | | NO: 433] | NO: 487] | |
| ET200- | GGSFSGYY | INHSGST | ARLYEGGYHGWGSWLSSDS | SSNIGSNY | SNN | AAWDDSLSGYV |
| 069 | [SEQ ID | [SEQ ID | [SEQ ID NO: 489] | [SEQ ID | [SEQ ID | [SEQ ID NO: 491] |
| | NO: 309] | NO: 310] | | NO: 490] | NO: 313] | |
| ET200- | GGSFSGYY | INHSGST | AREGAFDAFDI | SSNIGSNT | SNN | AAWDDSLNGY |
| 078 | [SEQ ID | [SEQ ID | [SEQ ID NO: 492] | [SEQ ID | [SEQ ID | [SEQ ID NO: 493] |
| | NO: 309] | NO: 310] | | NO: 312] | NO: 313] | |
| ET200- | GFTFDDYA | ISWNSGSI | ANGDSNYYYGMDV | SSNIGSNY | RNN | AAWDDSLSGYL |
| 079 | [SEQ ID | [SEQ ID | [SEQ ID NO: 494] | [SEQ ID | [SEQ ID | [SEQ ID NO: 495] |
| | NO: 403] | NO: 404] | | NO: 490] | NO: 349] | |
| ET200- | GFTFDDYA | ISGDGGST | AKDRAAAGYYYYGMDV | SSDIGGYNY | DVS | ISYTRTWNPYV |
| 081 | [SEQ ID | [SEQ ID | [SEQ ID NO: 497] | [SEQ ID | [SEQ ID | [SEQ ID NO: 499] |
| | NO: 403] | NO: 496] | | NO: 498] | NO: 344] | |
| ET200- | GFTFNDYA | ISWSGNNI | AKDSIRYGITWGGFDY | KLGEKY | QDT | QAWDRGVV |
| 097 | [SEQ ID | [SEQ ID | [SEQ ID NO: 502] | [SEQ ID | [SEQ ID | [SEQ ID NO: 505] |
| | NO: 500] | NO: 501] | | NO: 503] | NO: 504] | |
| ET200- | GFTFDDYA | INWDGGST | AKGMGLRAFDY | SNNVGNLG | RNN | SAWDSSLSA |
| 098 | [SEQ ID | [SEQ ID | [SEQ ID NO: 507] | [SEQ ID | [SEQ ID | [SEQ ID NO: 509] |
| | NO: 403] | NO: 506] | | NO: 508] | NO: 349] | |
| ET200- | GYTFSWYA | INAGNGNT | ARPDNYGSGGDVFDI | SSNIGSNT | SND | ASWDDSLNGRYV |
| 099 | [SEQ ID | [SEQ ID | [SEQ ID NO: 512] | [SEQ ID | [SEQ ID | [SEQ ID NO: 514] |
| | NO: 510] | NO: 511] | | NO: 312] | NO: 513] | |
| ET200- | GFTFSSYE | ISSSGSTI | ARWDYGMDV | SGSIASNF | EDN | QSYDTSNVV |
| 100 | [SEQ ID | [SEQ ID | [SEQ ID NO: 517] | [SEQ ID | [SEQ ID | [SEQ ID NO: 519] |
| | NO: 515] | NO: 516] | | NO: 518] | NO: 319] | |
| ET200- | GYTFTWYA | INAGSGNT | ARPNNYGSGGDVFDI | NSNIGSNY | SSS | ATWDDSLNA |
| 101 | [SEQ ID | [SEQ ID | [SEQ ID NO: 522] | [SEQ ID | [SEQ ID | [SEQ ID NO: 524] |
| | NO: 520] | NO: 521] | | NO: 348] | NO: 523] | |
| ET200- | GYTFTNYA | INGGNGNT | AKPEETAGTIHFDY | SSNIGNNY | DNN | GTWDSSLSAYV |
| 102 | [SEQ ID | [SEQ ID | [SEQ ID NO: 527] | [SEQ ID | [SEQ ID | [SEQ ID NO: 528] |
| | NO: 525] | NO: 526] | | NO: 406] | NO: 398] | |
| ET200- | GGTFSSYA | IIPIFGTA | AGEGYYDSSGYSNGDAFDI | SGSIASNY | EDN | QSYDSTITV |
| 103 | [SEQ ID | [SEQ ID | [SEQ ID NO: 529] | [SEQ ID | [SEQ ID | [SEQ ID NO: 530] |
| | NO: 411] | NO: 412] | | NO: 318] | NO: 319] | |
| ET200- | GFTFSSYE | ISSSGSTI | ARWDYGMDV | SGSIANSNY | EDN | QSYDSSNVV |
| 104 | [SEQ ID | [SEQ ID | [SEQ ID NO: 517] | [SEQ ID | [SEQ ID | [SEQ ID NO: 531] |
| | NO: 515] | NO: 516] | | NO: 318] | NO: 319] | |
| ET200- | GFTFDDYA | ISWNSGSI | AKDRGGGVIVKDAFDI | RLTNKY | EDA | QAWDSSVVV |
| 105 | [SEQ ID | [SEQ ID | [SEQ ID NO: 532] | [SEQ ID | [SEQ ID | [SEQ ID NO: 535] |
| | NO: 403] | NO: 404] | | NO: 533] | NO: 534] | |
| ET200- | GYNFLNYG | ISTYTGNT | ARQQGGGWYDV | VSNIGSGA | SYN | ATWDDSVNG |
| 106 | [SEQ ID | [SEQ ID | [SEQ ID NO: 536] | [SEQ ID ] | [SEQ ID | [SEQ ID NO: 430] |
| | NO: 425] | NO: 426] | | NO: 428 | NO: 429] | |
| ET200- | GYTFTSYT | ISTYNGLT | VREGSPDYGDFASFDY | NFNVGNND | DNN | GTWDSSLNTGGV |
| 107 | [SEQ ID | [SEQ ID | [SEQ ID NO: 539] | [SEQ ID | [SEQ ID | [SEQ ID NO: 541] |
| | NO: 537] | NO: 538] | | NO: 540] | NO: 398] | |
| ET200- | GYTFTSYT | ISTYNGLT | VREGSPDYGDFASFDY | SSNIGNNY | DNN | GTWDTSLSGFYV |
| 108 | [SEQ ID | [SEQ ID | [SEQ ID NO: 539] | [SEQ ID | [SEQ ID | [SEQ ID NO: 542] |
| | NO: 537] | NO: 538] | | NO: 406] | NO: 398] | |
| ET200- | GGTFSSYA | IIPIFGTA | ARDPAYGDYEYDAFDI | TSNIGSNT | NNN | ATWDDSLSGVV |
| 109 | [SEQ ID | [SEQ ID | [SEQ ID NO: 543] | [SEQ ID | [SEQ ID | [SEQ ID NO: 545] |
| | NO: 411] | NO: 412] | | NO: 544] | NO: 448] | |
| ET200- | GGTFSSYA | IIPIFGTA | ARGAGFDAFDI | SSNIGTNG | TND | AVWDHSLNGPV |
| 110 | [SEQ ID | [SEQ ID | [SEQ ID NO: 546] | [SEQ ID | [SEQ ID | [SEQ ID NO: 549] |
| | NO: 411] | NO: 412] | | NO: 547] | NO: 548] | |
| ET200- | GGSFSGYY | INHSGST | AREGLDAFDI | SSNIGSNT | SNN | AAWDDSLNGYV |
| 111 | [SEQ ID | [SEQ ID | [SEQ ID NO: 550] | [SEQ ID | [SEQ ID | [SEQ ID NO: 314] |
| | NO: 309] | NO: 310] | | NO: 312] | NO: 313] | |
| ET200- | GGSFSGYY | INHSGST | AREGLDAFDI | SSNIGSNT | SND | AAWDDSLNGYV |
| 112 | [SEQ ID | [SEQ ID | [SEQ ID NO: 550] | [SEQ ID | [SEQ ID | [SEQ ID NO: 314] |
| | NO: 309] | NO: 310] | | NO: 312] | NO: 513] | |
| ET200- | GYSFTSYT | VSTYNGLR | VREGSPDYGDFAAFDY | SSNIGNNY | DNN | GTWDSSLSAAYV |
| 113 | [SEQ ID | [SEQ ID | [SEQ ID NO: 553] | [SEQ ID | [SEQ ID | [SEQ ID NO: 554] |
| | NO: 551] | NO: 552] | | NO: 406] | NO: 398] | |
| ET200- | GGSFSGYY | INHSGST | ARDGGGYFDY | RSNIGTNI | GS | AAWDDSLNGPA |
| 114 | [SEQ ID | [SEQ ID | [SEQ ID NO: 555] | [SEQ ID | [SEQ ID | [SEQ ID NO: 558] |
| | NO: 309] | NO: 310] | | NO: 556] | NO: 557] | |
| ET200- | GYTFTSYG | INPGSGET | ATGLIRGASDAFNI | SSNGIARYD | ANY | QSYDSSVSAWV |
| 115 | [SEQ ID | [SEQ ID | [SEQ ID NO: 561] | [SEQ ID | [SEQ ID | [SEQ ID NO: 564] |
| | NO: 559] | NO: 560] | | NO: 562] | NO: 563] | |
| ET200- | GFTFSSYS | TYYRSKWYN | ARERSGWKGFDY | KLGDKF | QDT | QTWASGIVV |
| 116 | [SEQ ID | [SEQ ID | [SEQ ID NO: 567] | [SEQ ID | [SEQ ID | [SEQ ID NO: 569] |
| | NO: 565] | NO: 566] | | NO: 568] | NO: 504] | |
| ET200- | GGSFSGYY | ISGSGGST | AKWGPFQDAFDI | QSLLERNAYNY | LGS | MQALQAPFT |
| 117 | [SEQ ID | [SEQ ID | [SEQ ID NO: 570] | [SEQ ID | [SEQ ID | [SEQ ID NO: 573] |
| | NO: 372] | NO: 475] | | NO: 571] | NO: 572] | |
| ET200- | GFTFDDYA | ISWNSGSI | AKARWTAVASDHHFDY | SSDVGGYNY | EVS | SSYTSSSTPYV |
| 118 | [SEQ ID | [SEQ ID | [SEQ ID NO: 574] | [SEQ ID | [SEQ ID | [SEQ ID NO: 576] |
| | NO: 403] | NO: 404] | | NO: 343] | NO: 575] | |
| ET200- | GGTFSSYA | IIPIFGTA | ARDWDYMDV | SSNIGSNT | SNN | AAWDDSLNGYV |
| 119 | [SEQ ID | [SEQ ID | [SEQ ID NO: 577] | [SEQ ID | [SEQ ID | [SEQ ID NO: 314] |
| | NO: 411] | NO: 412] | | NO: 312] | NO: 313] | |
| ET200- | GYTFTSYG | ISAYNGNT | ARDLSRGANPHYYYYYGMDV | SSNIGSNT | SNN | AAWDDSLNGYV |
| 120 | [SEQ ID | [SEQ ID | [SEQ ID NO: 578] | [SEQ ID | [SEQ ID | [SEQ ID NO: 314] |
| | NO: 346] | NO: 327] | | NO: 312] | NO: 313] | |
| ET200- | GYTLTELS | FDPEDGET | ATESNLSRHYYYYGMDV | RSNIGAGYD | GNS | QSYDNTVRESPYV |
| 121 | [SEQ ID | [SEQ ID | [SEQ ID NO: 579] | [SEQ ID | [SEQ ID | [SEQ ID NO: 581] |
| | NO: 436] | NO: 388] | | NO: 580] | NO: 438] | |
| ET200- | GYTFTGYY | INPNSGGT | ARDYGYYGSGSYSSGPLYYYYGMDV | SSNIGSNS | SNN | AAWDDSLNGYV |
| 122 | [SEQ ID | [SEQ ID | [SEQ ID NO: 584] | [SEQ ID | [SEQ ID | [SEQ ID NO: 314] |
| | NO: 582] | NO: 583] | | NO: 585] | NO: 313] | |
| ET200- | GYTFTSYG | ISAYNGNT | ARDLSRGANPHYYYYYGMDV | SSNIGSNT | NND | AAWDDSLNGYV |
| 123 | [SEQ ID | [SEQ ID | [SEQ ID NO: 578] | [SEQ ID | [SEQ ID | [SEQ ID NO: 314] |
| | NO: 346] | NO: 327] | | NO: 312] | NO: 586] | |
| ET200- | GFTFDDYA | ISWNSGSI | AKDITYGSGSYGAFDI | DIGSKS | DDS | QVWDSSSDHYV |
| 124 | [SEQ ID | [SEQ ID | [SEQ ID NO: 587] | [SEQ ID | [SEQ ID | [SEQ ID NO: 375] |
| | NO: 403] | NO: 404] | | NO: 588] | NO: 685] | |
| ET200- | GGTFSSNS | IFPILGIT | ARGNYQWYDAFDI | SGSIASNY | EDN | QSYDSTSVL |
| 125 | [SEQ ID | [SEQ ID | [SEQ ID NO: 591] | [SEQ ID | [SEQ ID | [SEQ ID NO: 592] |
| | NO: 589] | NO: 590] | | NO: 318] | NO: 319] | |

TABLE 230

| CDR | CDR SEQUENCE | SEQ ID NO |
|---|---|---|
| V_{H}CDR1 | IFGLH | SEQ ID NO: 923 |
| V_{H}CDR2 | YISGDSNTIYYADTVKG | SEQ ID NO: 924 |
| V_{H}CDR3 | NSYYALDY | SEQ ID NO: 925 |
| V_{L}CDR1 | RASSSVSSSYLH | SEQ ID NO: 926 |
| V_{L}CDR2 | STSNLAS | SEQ ID NO: 927 |
| V_{L}CDR3 | QQYSGYPWT | SEQ ID NO: 928 |
| V_{H}CDR1 | SFGMH | SEQ ID NO: 929 |
| V_{L}CDR2 | YISSGSNNIYFADTVKG | SEQ ID NO: 930 |
| V_{L}CDR3 | SEYYGSSHMDY | SEQ ID NO: 931 |
| V_{L}CDR1 | KASQNVGTNVA | SEQ ID NO: 932 |
| V_{L}CDR2 | SATYRNS | SEQ ID NO: 933 |
| V_{L}CDR3 | QQYNRYPYT | SEQ ID NO: 934 |

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1—FcRL5 Expression in various tissues

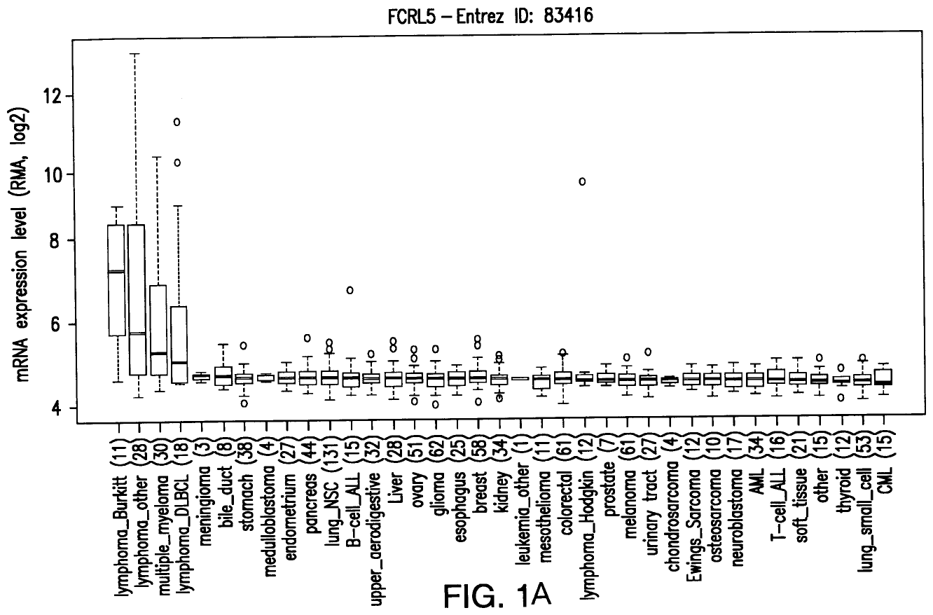

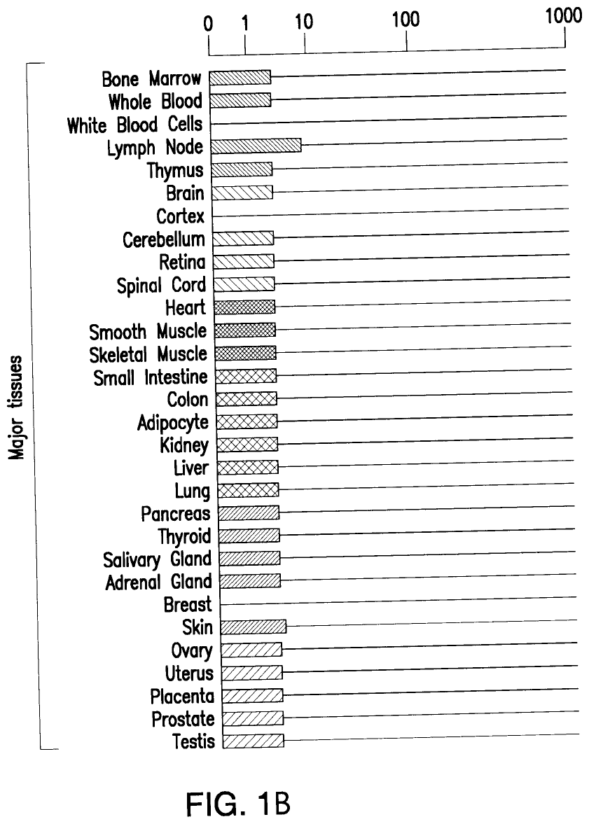

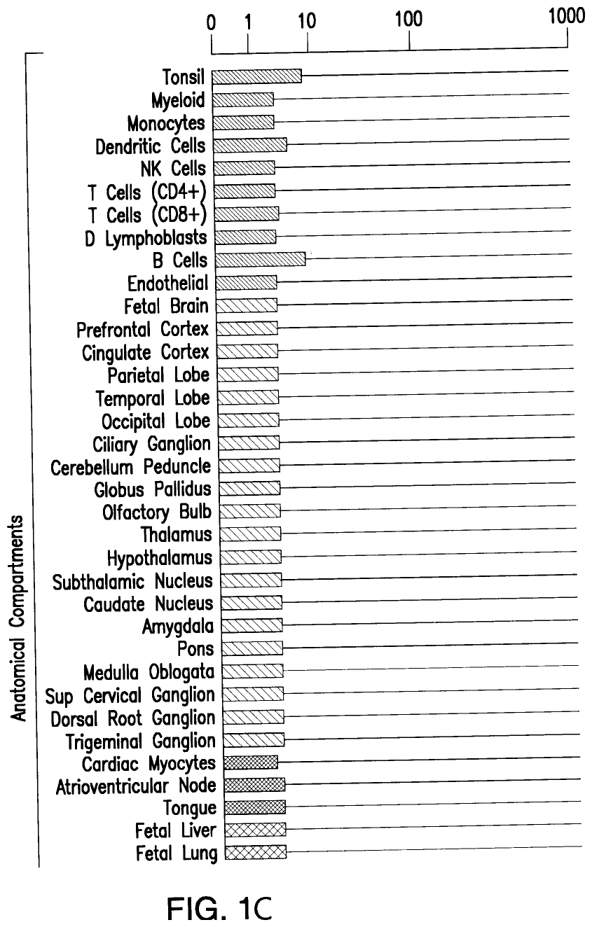

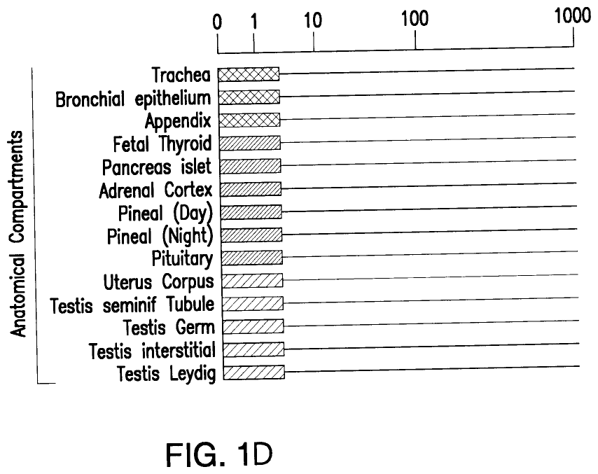

The expression of human FcRL5 was assessed and evaluated in various tissues. As shown in FIGS. 1A-1D, human FcRL5 was highly expressed in lymphoma and multiple myeloma, but not in other tissues. Top panel of FIGS. 1A-1D shows differential expression of human FcRL5 in tumor cell lines from the Cancer Cell Line Encyclopedia (CCLE). The bottom panel of FIGS. 1A-1D shows differential expression of human FcRL5 in normal tissue from BioGPS. As shown in FIGS. 1A-1D, human FcRL5 expression is limited to MM and lymphoma compared to other malignant cells. Normal expression appeared limited to B-cells and plasma cells. Potential FcRL5 targeted CAR T cell eradication of these normal cell types may not have significant adverse effects based on inventors' patient experience with CD19 targeted CAR T cells. Any lack of physiologic antibody production can be addressed with intravenous immunoglobulin treatment.

### Example 2—Selection of ScFv specific for FcRL5 using a fully human phage display library

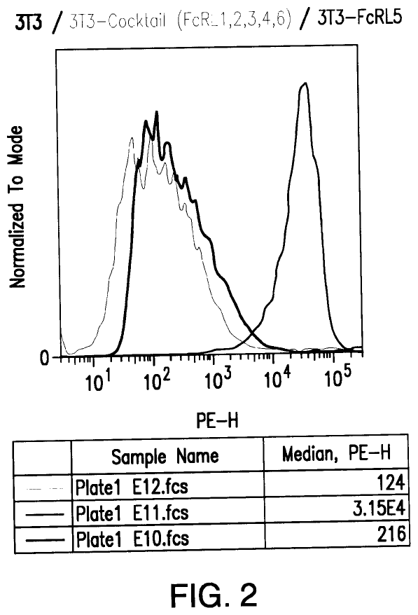

Phage display selection against FcRL5 was conducted using a cell panning strategy with 31 human scFv naïve and semi-synthetic phage sub-libraries. FcRL5 overexpressing 3T3 cells were used in positive panning, FcRL1, 2, 3, 4 and 6 overexpressing 3T3 cells (5 cell lines in total) were used in negative panning (FIG. 2). Bound clones were then eluted and used to infect E. Coli XL1-Blue. The scFv phage clones expressed in the bacteria were purified as previously described (Yasmina, et al. 20 Probing the binding mechanism and affinity of tanezumab, a recombinant humanized anti-NGF monoclonal antibody, using a repertoire of biosensors. Protein Science 2008, 17(8):1326-1335; Roberts, et al. Vaccination with CD20 peptides induces a biologically active, specific immune response in mice. Blood 2002, 99(10):3748-3755). Panning was performed for about 3 to about 4 cycles to enrich scFv phage clones that bind to FcRL5 specifically. Positive clones were determined by an ELISA method against His-tag FcRL5.

Positive clones were further tested for their binding to FcRL5 on live cell surfaces by flow cytometry, using FcRL5-overexpressing cell lines, 3T3 and Raji. The cells were then washed, and the staining was performed using the following steps: the cells can be first stained with purified scFv phage clones, and followed by staining with a mouse anti-M13 mAb, and finally the horse anti-mouse Ig's conjugate to PE. Each step of the staining was completed between 30-60 minutes on ice and the cells were washed twice between each step of the staining.

76 unique clones that were specific for FcRL5 were identified and confirmed by screening (see Tables 1-229 and FIG. 2).

### Example 3—Selection of ScFv specific for domain 9 of FcRL5

FcRL5 contains 9 extracellular immunoglobulin-like domains (domains 1-9) and can be present within a cell in a soluble isoform, a glycosyl-phosphotidyl inositol (GPI)-anchor type isoform and a transmembrane-type isoform (FIG. 3A). As shown in FIG. 2A, the transmembrane-type isoform of FcRL5 has domain 9; whereas, the soluble isoform and the GPI-anchor type isoform do not.

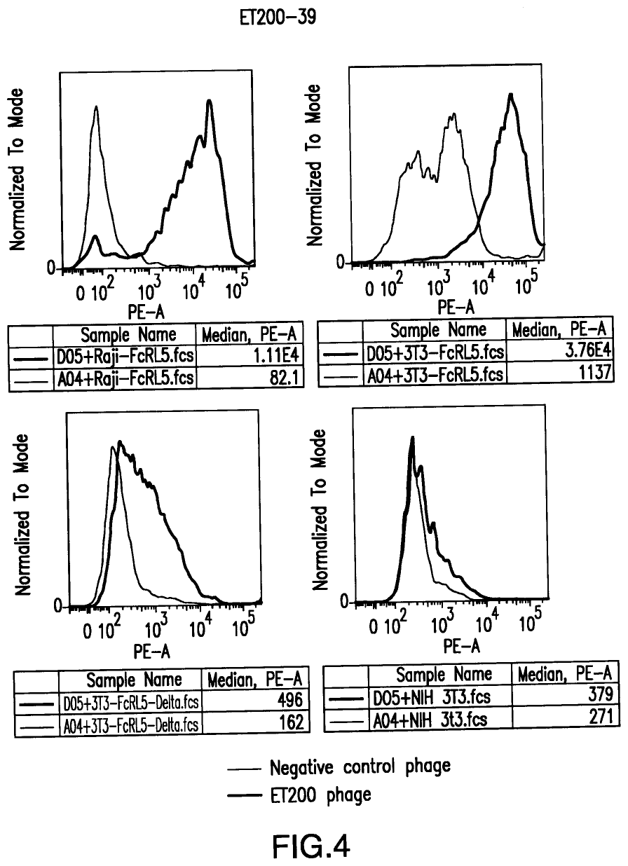

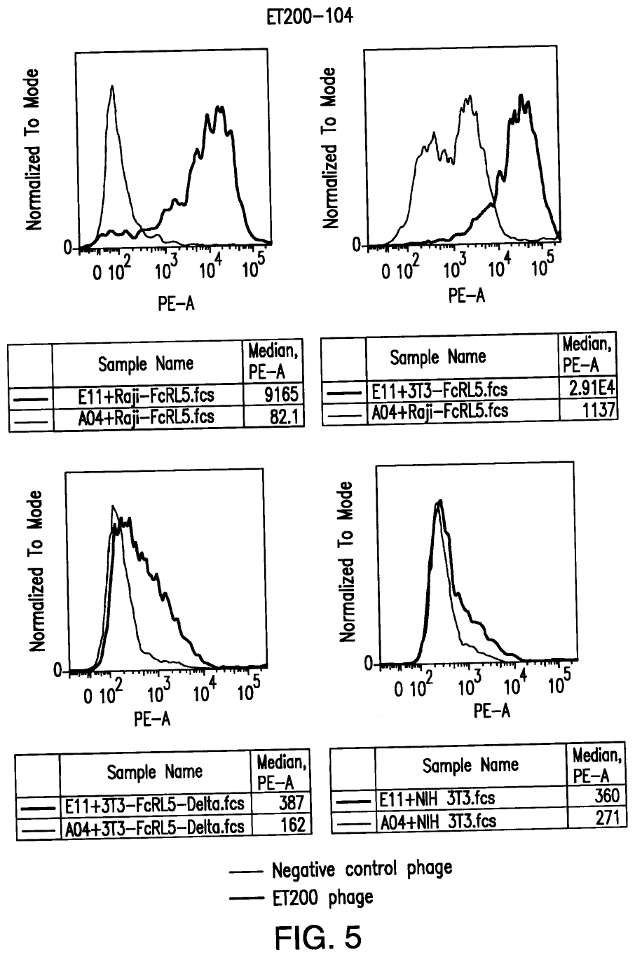

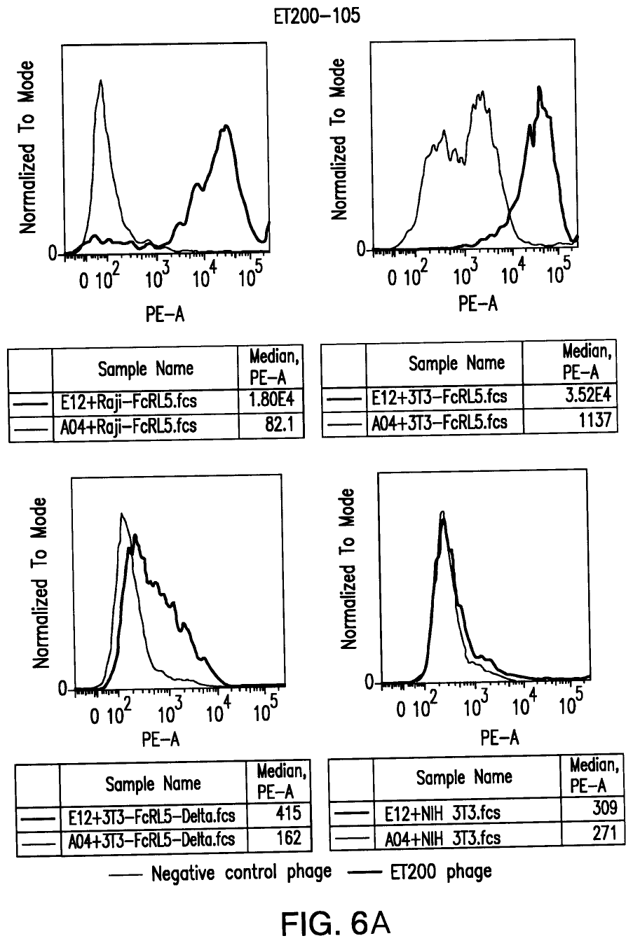

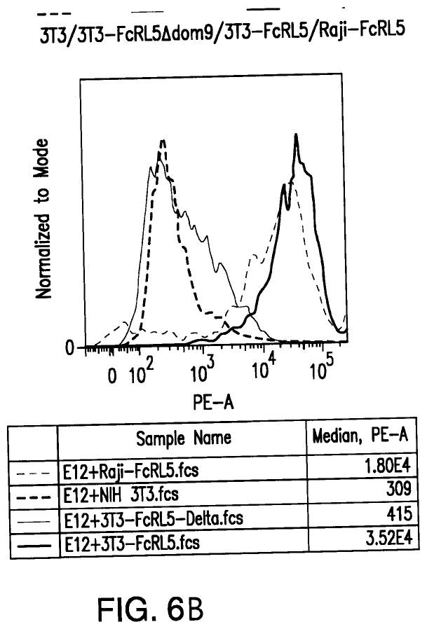

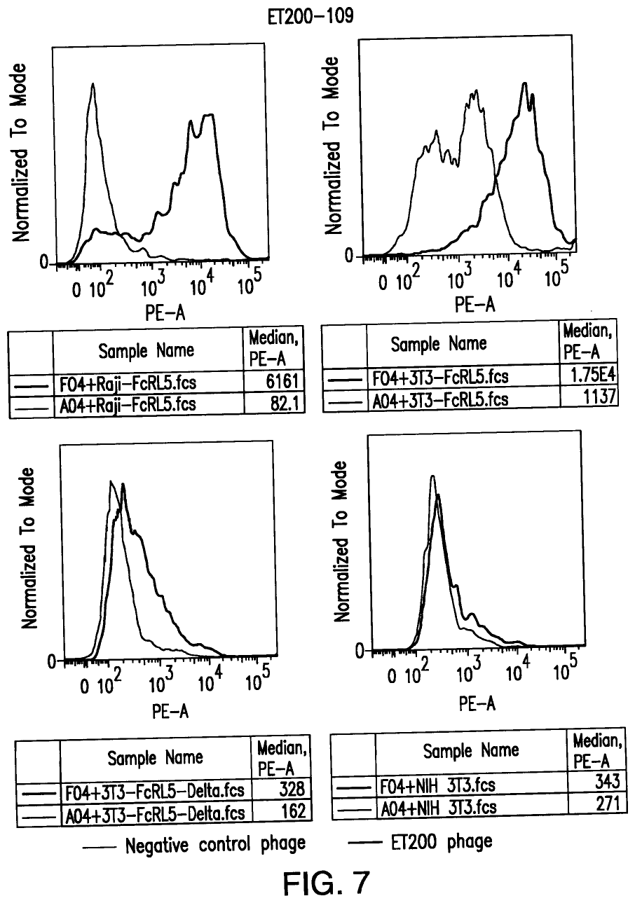

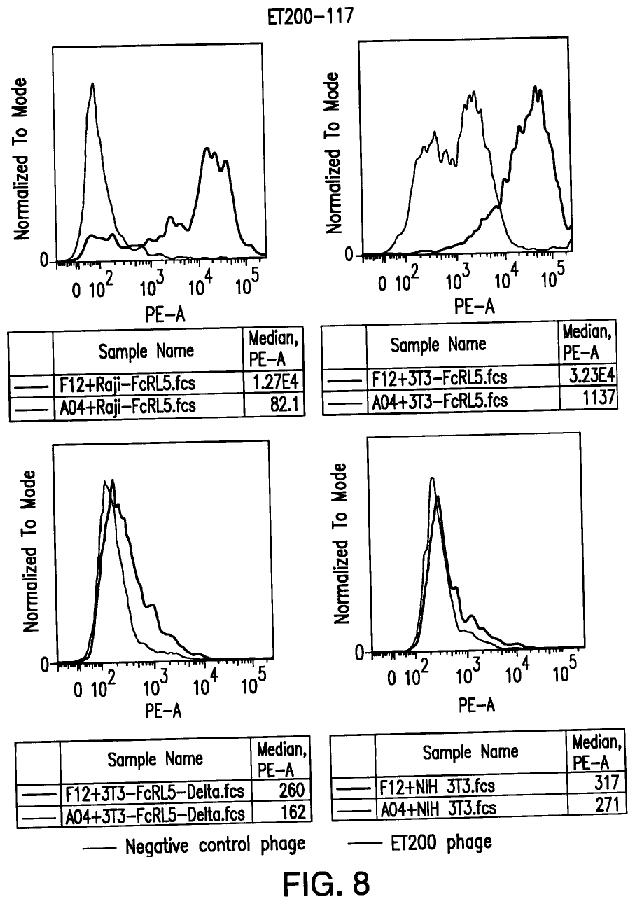

To test if the scFvs were specific to domain 9 of FcRL5, the 76 clones were further screened on 3T3 cells overexpressing an vector encoding FcRL5 with a domain 9 deletion (FcRL5Δdom9) and further screened on Raji cells overexpressing full-length FcRL5 (FIG. 4B-D). Some clones showed either reduced or diminished binding towards FcRL5-domain 9 deletion-overexpressing 3T3 cells compared to binding towards FcRL5-overexpressing 3T3 cells. FIGS. 4, 5, 6A-6B, 7 and 8 shows the specificity of ET200-39, ET200-104, ET200-105, ET200-109 and ET200-117 for domain 9 of FcRL5, respectively.

### Example 4—Construct of murine FcRL5-specific CARs

For generation of a scFv targeting human FcRL5, two commercially available mouse hybridomas binding different extracellular epitopes on human FcRL5 (Franco (2013); Ise et al. (2005); Ise et al. (2006)) were obtained. From these hybridomas, two scFvs targeting human FcRL5 were obtained. One scFv was generated by synthesizing the heavy and light chain variable region of murine anti-human FcRL5 antibody F56 as described in Ise et al. (2005) with a G4S linker having the amino acid sequence of SEQ ID NO:897. A second scFv was generated by synthesizing the heavy and light chain variable region of murine anti-human FcRL5 antibody F119 as described in Ise et al. (2005) with a G4S linker having the amino acid sequence of SEQ ID NO:897. Two FcRL5 CARs were generated: F56 FcRL5-28z CAR and F119 FcRL5-28z CAR. F56 FcRL5-28z CAR and F119 FcRL5-28z CAR have similar structure, e.g., each has a transmembrance domain comprising a CD28 polypeptide, and an intracellular domain comprising a CD3 polypeptide and a co-stimulatory signaling region that comprises a CD28 polypeptide, as shown in FIG. 9A. F56 FcRL5-28z CAR comprises an scFv derived from antibody F56, and F119 FcRL5-28z CAR comprises an scFv derived from antibody F119. Each of F56 FcRL5-28z CAR and F119 FcRL5-28z CAR were cloned into a retroviral vector 293galv9. Human T cells (unselected (CD4 and CD8) human T cells from a healthy donor) were transduced with each of F56 FcRL5-28z CAR and F119 FcRL5-28z CAR such that the T cells expressed F56 FcRL5-28z CAR or F119 FcRL5-28z CAR.

### Example 5—Construct of human FcRL5-specific CARs

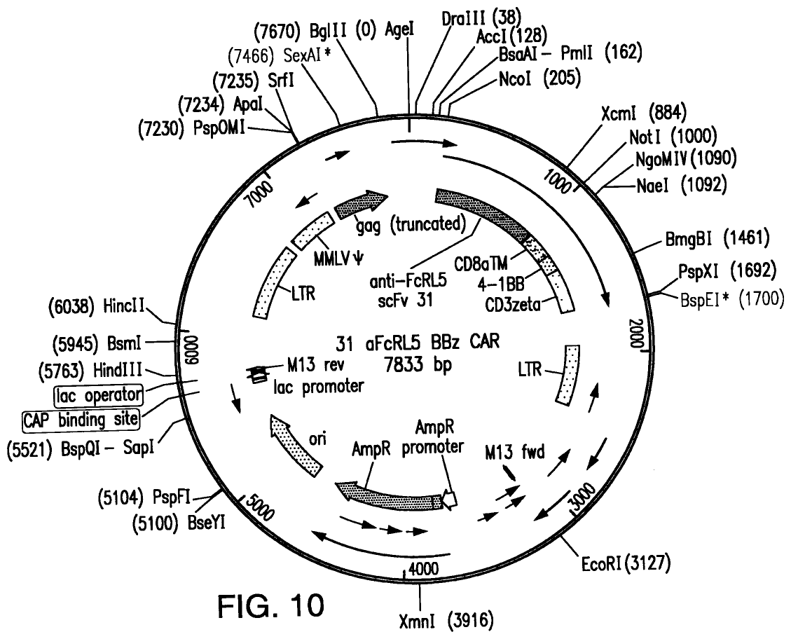

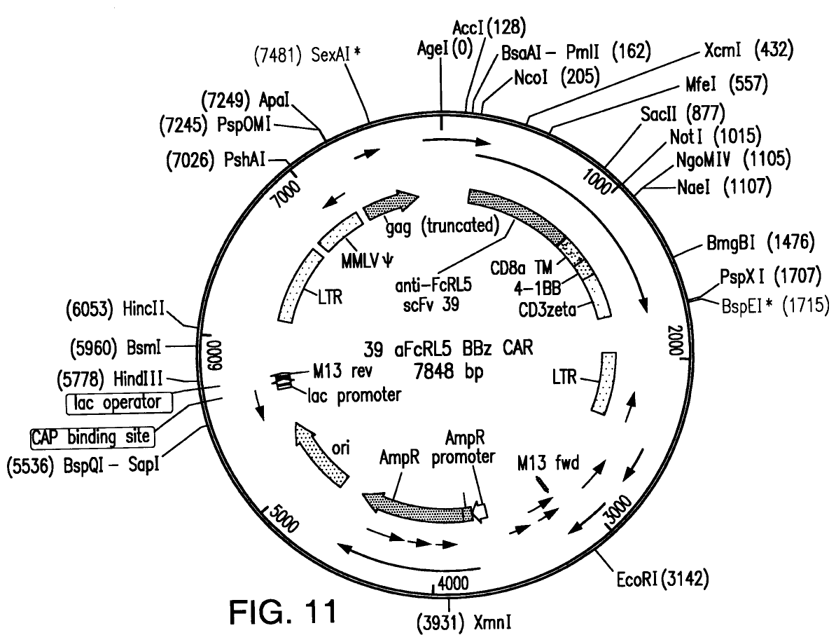

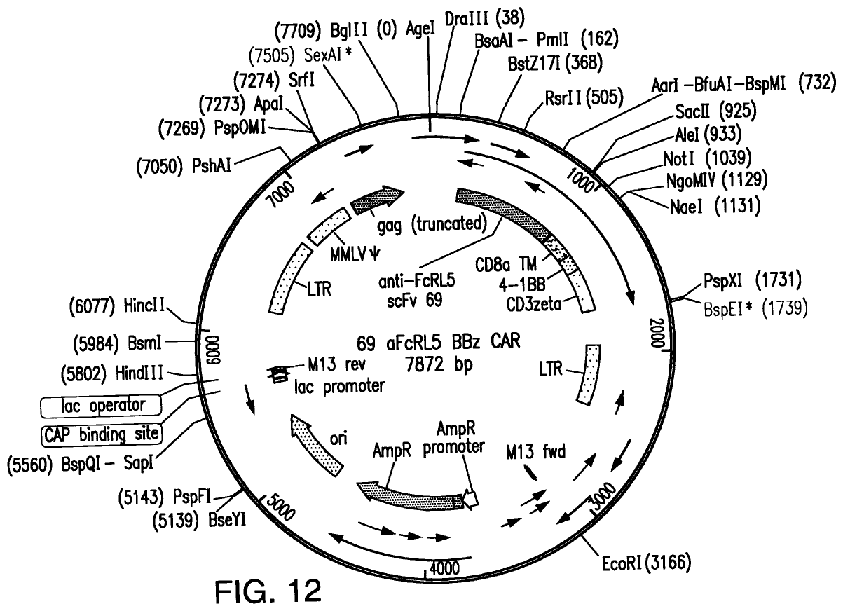

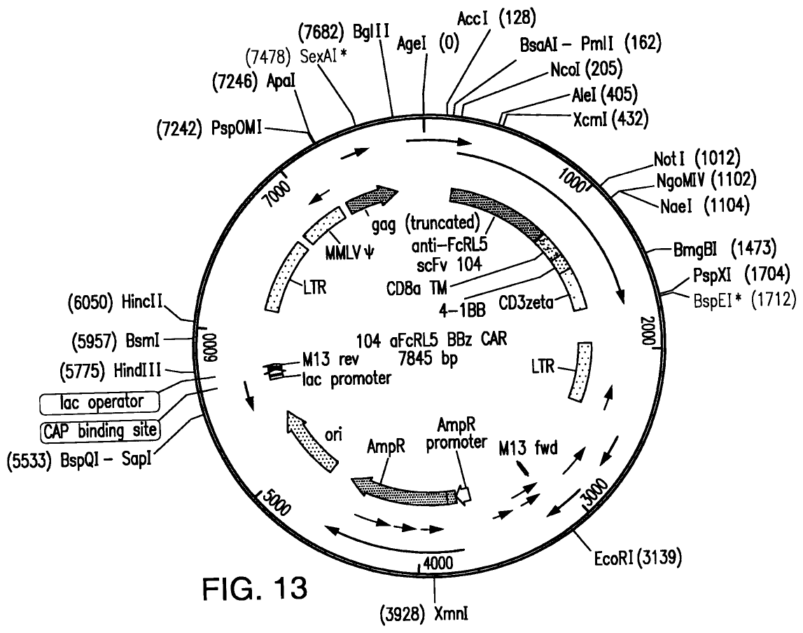

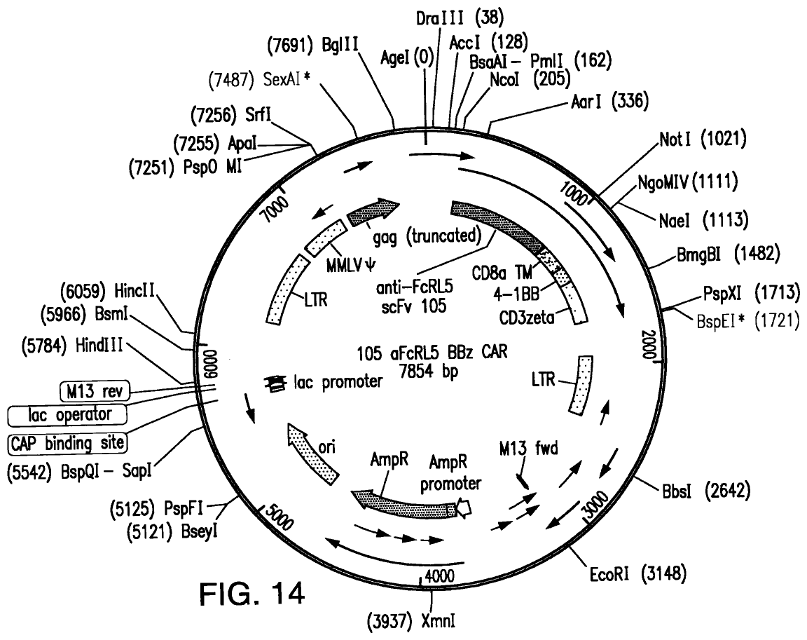

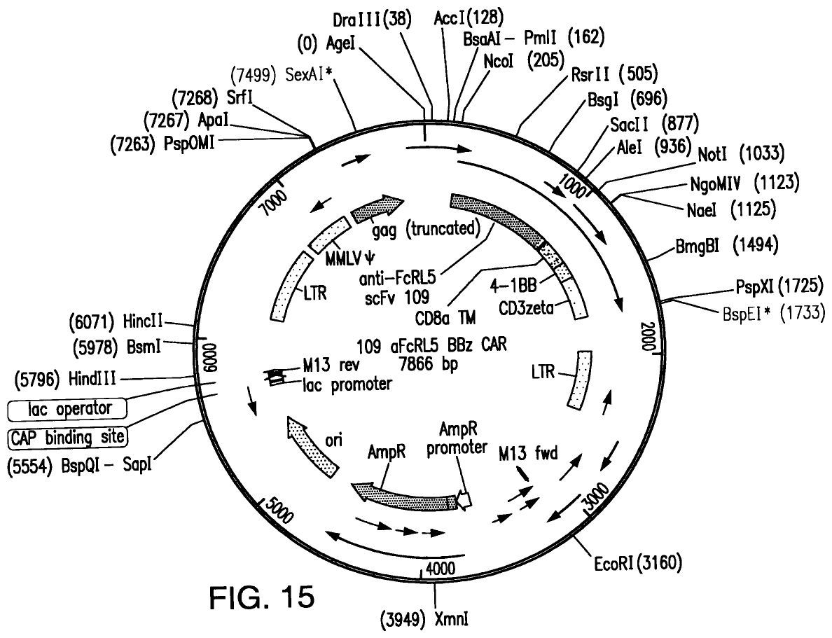

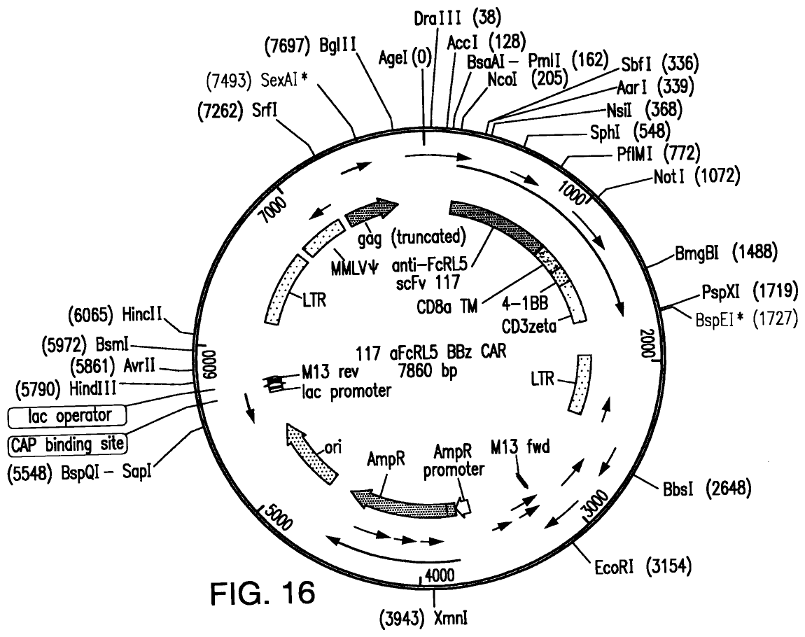

This Example discloses the generation of CARs targeting human FcRL5 using the fully human scFvs described herein. ET200-31 scFv, ET200-39 scFv, ET200-69 scFv, ET200-104 scFv, ET200-105 scFv, ET200-109 scFv and ET200-117 scFvs were used to generate second generation FcRL5-targeted CARs designated as 31 FcRL5 BBz CAR, 39 FcRL5 BBz CAR, 69 FcRL5 BBz CAR, 10⁴ FcRL5 BBz CAR, 105 FcRL5 BBz CAR, 109 FcRL5 BBz CAR and 117 FcRL5 BBz CAR with either a 4-1BB co-stimulatory domain and a CD3 polypeptide (see FIGS. 9B-16). The FcRL5-targeted 4-1BB containing CARs have a similar structure, e.g., each has a CD8a polypeptide transmembrane domain, and an intracellular domain comprising co-stimulatory signaling region consisting of a CD3 polypeptide and a 4-1BB polypeptide, as shown in FIG. 9B. Each of these FcRL5-targeted CARs were cloned into an SFG retroviral vector, as an example the 4-1BB containing CAR vectors are shown in FIGS. 10-16. These viral vectors are then transduced into HEK 293galv9 viral packaging cells in order to generate a stable packaging line for generation of CAR+ T cells.

### Example 6—Expression of human FcRL5-specific CARs in T cells

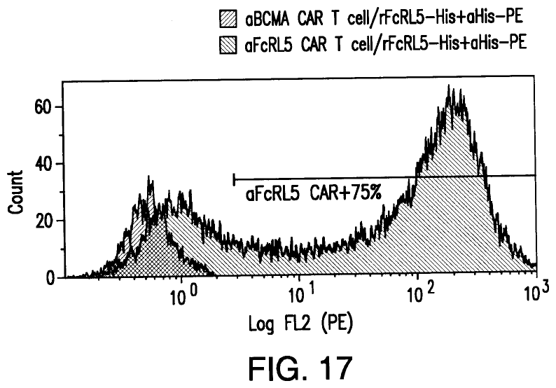

Human primary T cells were transduced using supernatant from retroviral galv9 HEK 293 packaging cells to express a FcRL5-targeted 4-1BBz CAR (generated using the anti-FcRL5 ET200-10⁴ scFv). The cell surface expression of the FcRL5-targeted CAR was determined by binding to recombinant human FcRL5 which was modified to contain a His tag followed by the binding of a PE conjugated anti-HIS tag secondary antibody, as shown in FIG. 17. Cell surface detection was validated by flow cytometry. As shown in FIG. 17, the FcRL5-targeted CAR was expressed on the transduced T cells.

### Example 7—Cytotoxicity of human FcRL5-specific CARs

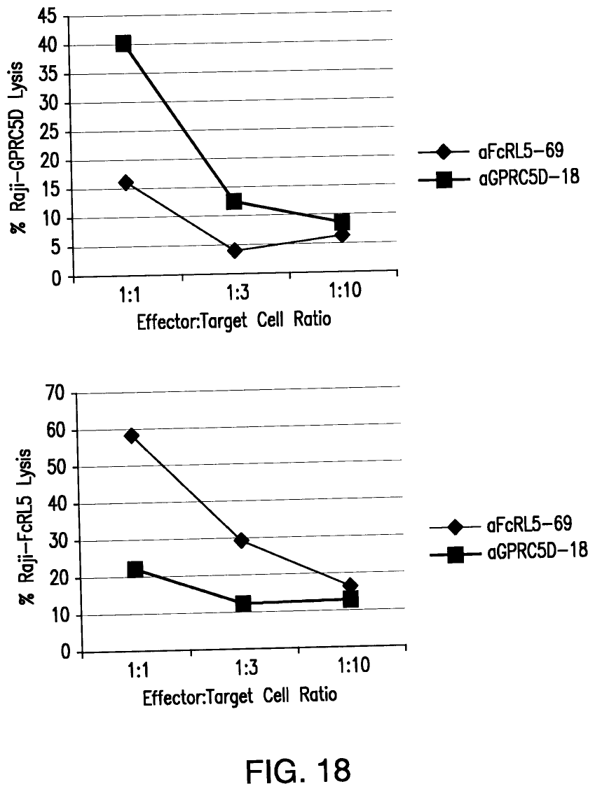

The cytotoxic effects of the FcRL5-targeted 4-1BB CAR T cells were analyzed using Raji (a Burkett's lymphoma cell line) cells transduced to express luciferase and FcRL5 antigen or the control 30 GPRC5D (an irrelevant antigen). T cells expressing the 4-1BB CAR generated using the anti-FcRL5 ET200-69 scFv were used in these experiments (referred to herein as “αFcRL5-69 BBz”). The number of live cells is determined after 36h co-culture by bioluminescent imaging (BLI). As shown in FIG. 18, FcRL5-targeted CAR T cells specifically lysed Raji cells transduced to express luciferase and FcRL5 antigen (FIG. 18A) but not control GPRC5D expressing Raji cells which are lysed by GPRC5D-targeted CAR T cells (FIG. 18B).

### Example 8—Cytokine secretion of human FcRL5-specific CARs

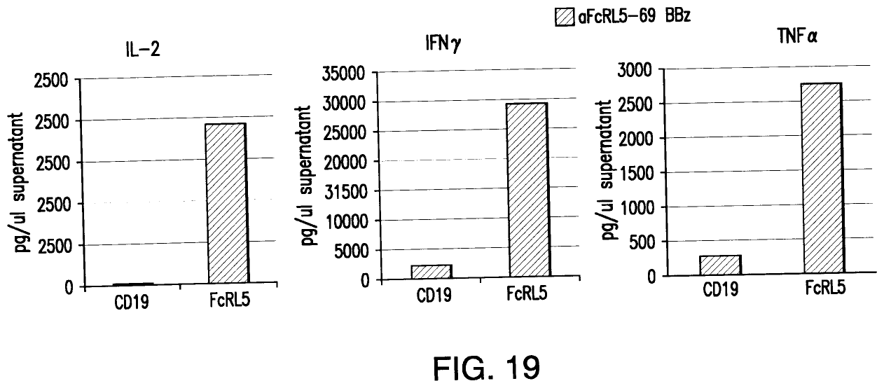

This Example describes the cytokine secretion of FcRL5-targeted CAR T cells. T cells expressing the 4-1BB CAR generated using the anti-FcRL5 ET200-69 scFv were used in these experiments. IL-2, INFγ, and TNFα secretion after 24h co-culture of FcRL5 targeted CAR T cells on a monolayer of 3T3 cells transduced with either FcRL5 or CD19 was assessed by Luminex multiplex analysis. As shown in FIG. 19, signaling through the CAR of FcRL5-targeted CAR T cells induced cytokine secretion consistent with T cell activation.

### Example 9—Proliferation of human FcRL5-specific CARs

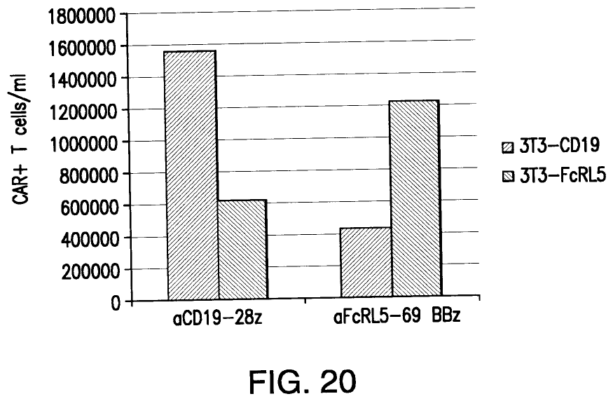

This Example describes the proliferation of FcRL5-targeted CAR T cells upon antigen 15 stimulation. T cells expressing the 4-1BB CAR generated using the anti-FcRL5 ET200-69 scFv was used in these experiments. 500,000 FcRL5- or CD19-targeted CAR+ T cells/ml were placed on a monolayer of 3T3 cells that have been transduced with either FcRL5 or CD19 (3T3-FcRL; 3T3-CD19). After 4 days, CAR+ T cells were stained and counted by flow cytometry with the inclusion of a known concentration of counting microbeads. As shown in FIG. 20, antigen stimulation of FcRL5-targeted CAR T cells induced proliferation. FcRL5-targeted CAR T cells co-cultured with 3T3-FcRL5 cells expanded to be 2.9 times greater then FcRL5-targeted CAR T cells co-cultured with 3T3-CD19 cells, while control CD19 targeted CAR T cells co-cultured with 3T3-CD19 cells similarly expanded to be 2.5 fold greater then CD19 targeted CAR T cells co-cultured with 3T3-FcRL5 cells (FIG. 20).

### Example 10—Epitope mapping of human anti-FcRL5 antibodies

Two anti-FcRL5 bispecific antibodies, ET200-104 and ET200-117, were analyzed by Pepscan to determine epitope specificity. See Table 239. The target protein is human FcRL5 comprising amino acids 1-851 of SEQ ID NO: 899.

TABLE 239

| Name | Origin | Concentration | Location |
|---|---|---|---|
| ET200-104 | human | 2.0 mg/ml | +4° C./22 |
| bispecific scFV | | | |
| ET200-117 | human | 1.6 mg/ml | +4° C./22 |
| bispecific scFV | | | |

### Methods

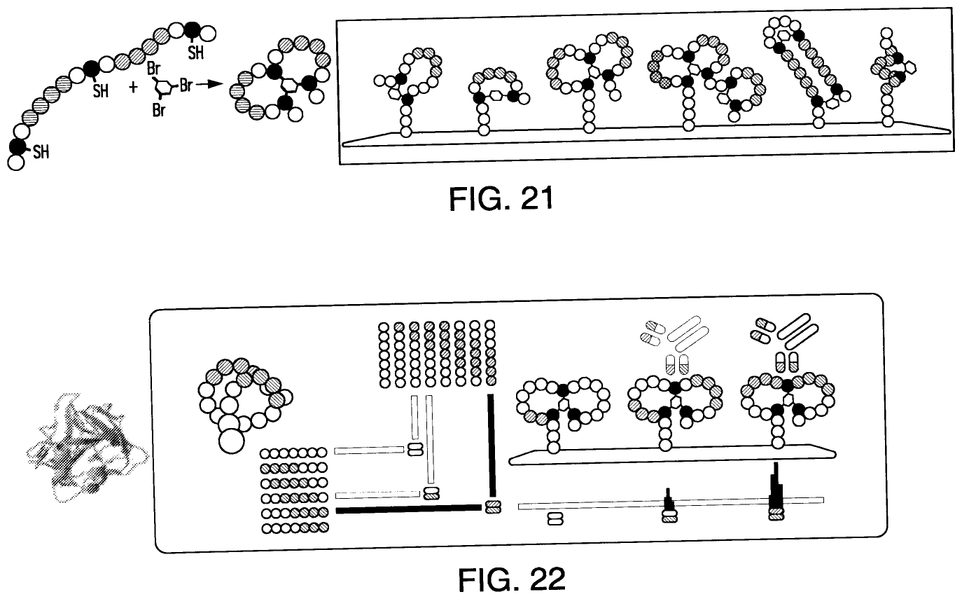

The principles of clips technology. CLIPS technology structurally fixes peptides into defined three-dimensional structures. This results in functional mimics of even the most complex binding sites. CLIPS technology is now routinely used to shape peptide libraries into single, double or triple looped structures as well as sheet- and helix-like folds (FIG. 21).

Combinatorial clips library screening in detail. CLIPS library screening starts with the conversion of the target protein into a library of up to 10,000 overlapping peptide constructs, using a combinatorial matrix design. On a solid carrier, a matrix of linear peptides is synthesized, which are subsequently shaped into spatially defined CLIPS constructs (FIG. 22). Constructs representing both parts of the discontinuous epitope in the correct conformation bind the antibody with high affinity, which is detected and quantified. Constructs presenting the incomplete epitope bind the antibody with lower affinity, whereas constructs not containing the epitope do not bind at all. Affinity information is used in iterative screens to define the sequence and conformation of epitopes in detail.

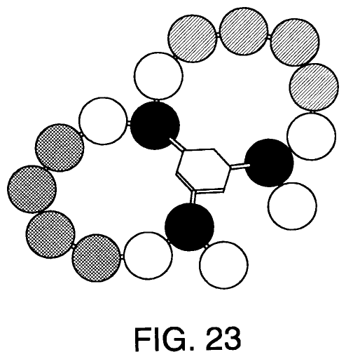

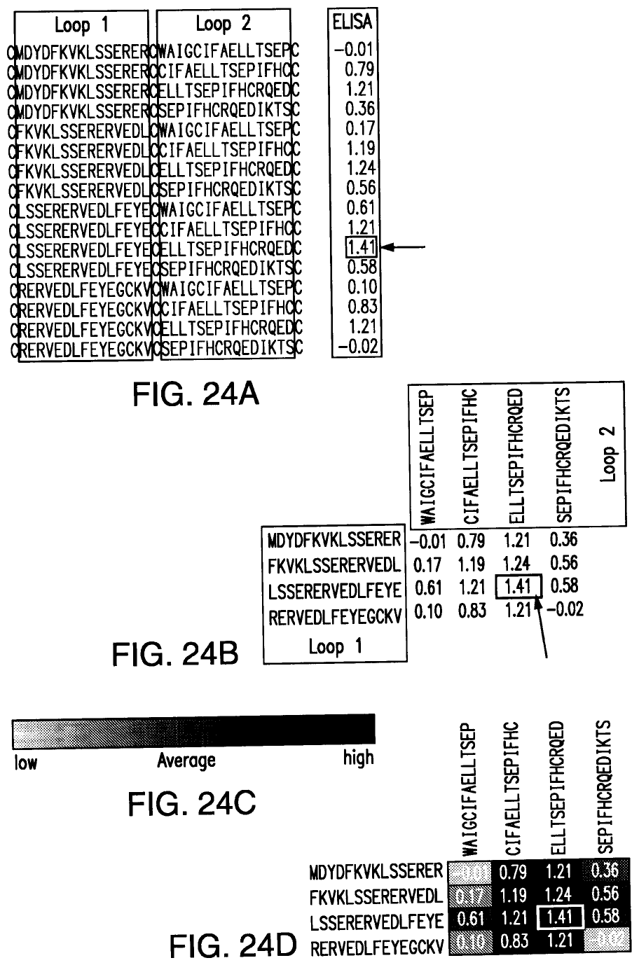

Heat map analysis. A heat map is a graphical representation of data where the values taken by a variable in a two-dimensional map are represented as colors. For double-looped CLIPS peptides, such a two-dimensional map can be derived from the independent sequences of the first and second loops. For example, the sequences of the 16 CLIPS peptides depicted in FIG. 24 are effectively permutations of 4 unique sub-sequences in loop 1 (colored in blue in FIG. 23) and 4 unique sub-sequences in loop 2 (colored in green in FIG. 23). Thus, the observed ELISA data (colored in red in FIG. 24A) can be plotted in a 4×4 matrix, where each X coordinate corresponds to the sequence of the first loop, and each Y coordinate corresponds to the sequence of the second loop. For instance, the ELISA value observed for CLIPS peptide CLSSERERVEDLFEYECELLTSEPIFHCRQEDC (indicated with an arrow in FIG. 23A) can be found at the third row, third column of FIG. 24B (indicated with an arrow and a red square). To further facilitate the visualization, ELISA values can be replaced with colors from a continuous gradient. In this case, extremely low values are colored in green, extremely high values are colored in red, and average values are colored in black (see FIG. 24C). For the aforementioned example, the average value is 0.71. When this color map is applied to the data matrix depicted in FIG. 24B, a color heat map is obtained (see FIG. 24D, the original data is still indicated for extra clarity).

Synthesis of peptides. To reconstruct epitopes of the target molecule a library of peptides was synthesized. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with Nhydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer). Synthesis of structural mimics was done using Pepscan's proprietary Chemically Linked Peptides on Scaffolds (CLIPS) technology. CLIPS technology allows to structure peptides into single loops, doubleloops, triple loops, sheet-like folds, helix-like folds and combinations thereof. CLIPS templates are coupled to cysteine residues. The side-chains of multiple cysteines in the peptides were coupled to one or two CLIPS templates. For example, a 0.5 mM solution of the P2 CLIPS (2,6-bis(bromomethyl)pyridine) was dissolved in ammonium bicarbonate (20 mM, pH 7.8)/acetonitrile (1:3(v/v)). This solution was added onto the peptide arrays. The CLIPS template bound to side-chains of two cysteines as present in the solid-phase bound peptides of the peptide-arrays (455 wells plate with 3 l wells). The peptide arrays were gently shaken in the solution for 30 to 60 minutes while completely covered in solution. Finally, the peptide arrays were washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes. The T3 CLIPS carrying peptides were made in a similar way but now with three cysteines.

ELISA Screening. The binding of antibody to each of the synthesized peptides was tested in a PEPSCAN-based ELISA. The peptide arrays were incubated with primary antibody solution (overnight at 4° C.). After washing, the peptide arrays were incubated with a 1/1000 dilution of an appropriate antibody peroxidase conjugate (SBA) for one hour at 25° C. After washing, the peroxidase substrate 2,2′-azino-di-3-ethylbenzthiazoline sulfonate (ABTS) and 2 μl/ml of 3 percent H₂O₂ were added. After one hour, the color development was measured. The color development was quantified with a charge coupled device (CCD) -camera and an image processing system.

Data processing. The values obtained from the CCD camera ranged from 0 to 3000 mAU, similar to a standard 96-well plate ELISA-reader. The results were quantified and stored into the Peplab database. Occasionally a well contained an air-bubble resulting in a false-positive value, the cards were manually inspected and any values caused by an air-bubble were scored as 0.

Synthesis quality control. To verify the quality of the synthesized peptides, a separate set of positive and negative control peptides was synthesized in parallel. These were screened with antibody 57.9 (ref. Posthumus et al., J. Virology, 1990, 64:3304-3309).

### Results

Screening. Antibody binding depends on a combination of factors, including concentration of the antibody and the amounts and nature of competing proteins in the ELISA buffer. Also, the pre-coat conditions (the specific treatment of the peptide arrays prior to incubation with the experimental sample) affected binding. These details are summed up in Table 240. For the Pepscan Buffer and Preconditioning (SQ), the numbers indicate the relative amount of competing protein (a combination of horse serum and ovalbumin).

TABLE 240
Screening conditions

| Label | Dilution | Sample Buffer | Pre-conditioning |
|---|---|---|---|
| ET200-104 | 8 μg/ml | PBS-Tween | PBS-Tween |
| ET200-117 | 3 μg/ml | PBS-Tween | 0.1% SQ |

Antibodies ET200-104 and ET200-117 were coated at 1 g/ml on a Nunc Maxisorp plate for ELISA and detected with Goat Anti-Human Ig-HRP (Southern Biotech; #2010/05), the same conjugate that is used in minicard screenings. For ET200-104 and ET200-117 signal >1 OD was obtained for some dilutions of the secondary Ab, indicating that the secondary antibody is well suited for detection of these mAbs.

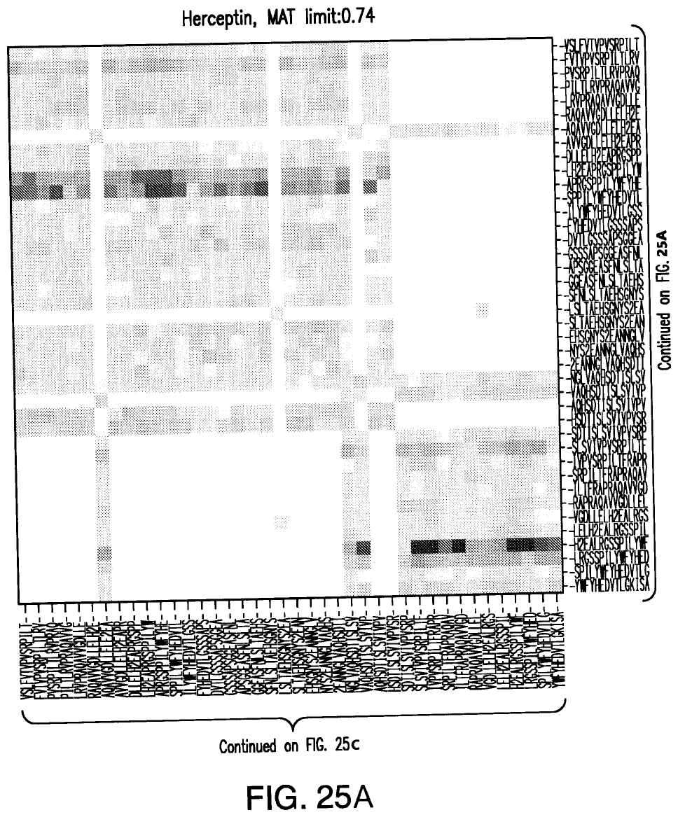

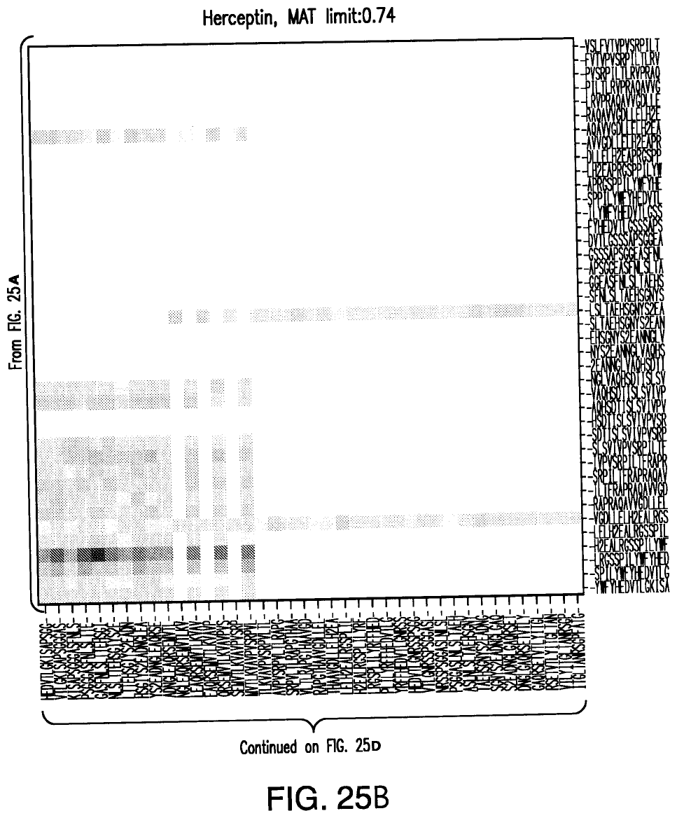

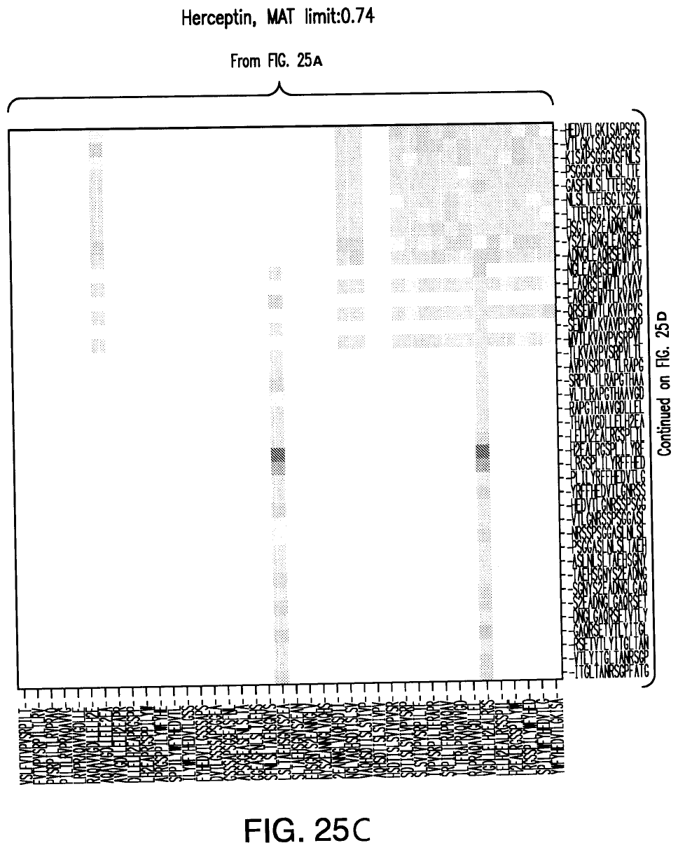

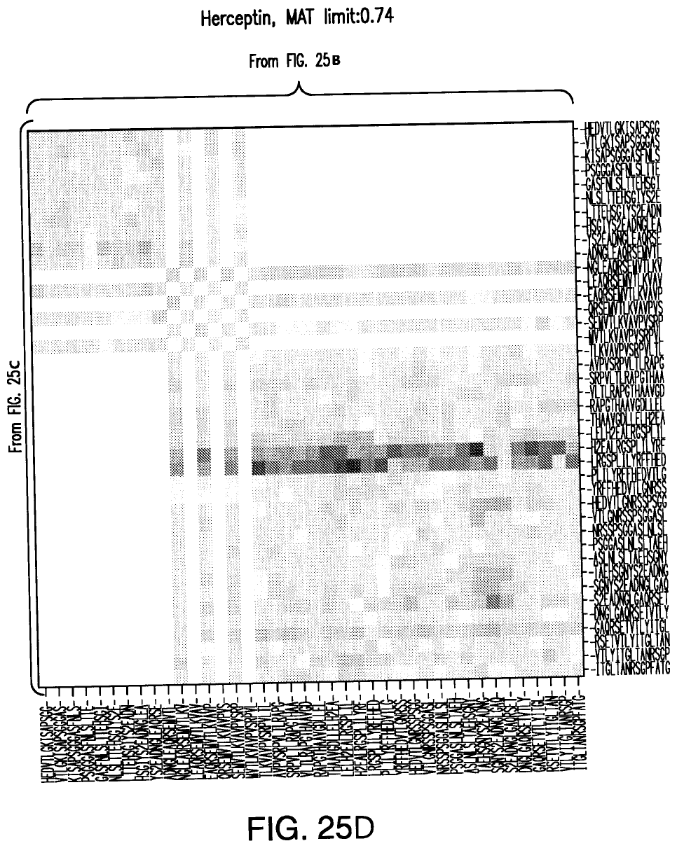

Herceptin was used as an internal negative control at high concentration in the absence of blocking buffer. Herceptin bound peptides with common sequences LRGSPLILYRF, LRGSSPILYWF and APRGSPPILYW (FIGS. 25A-25D). Peptides containing aforementioned sequences were excluded from epitope candidates for test samples.

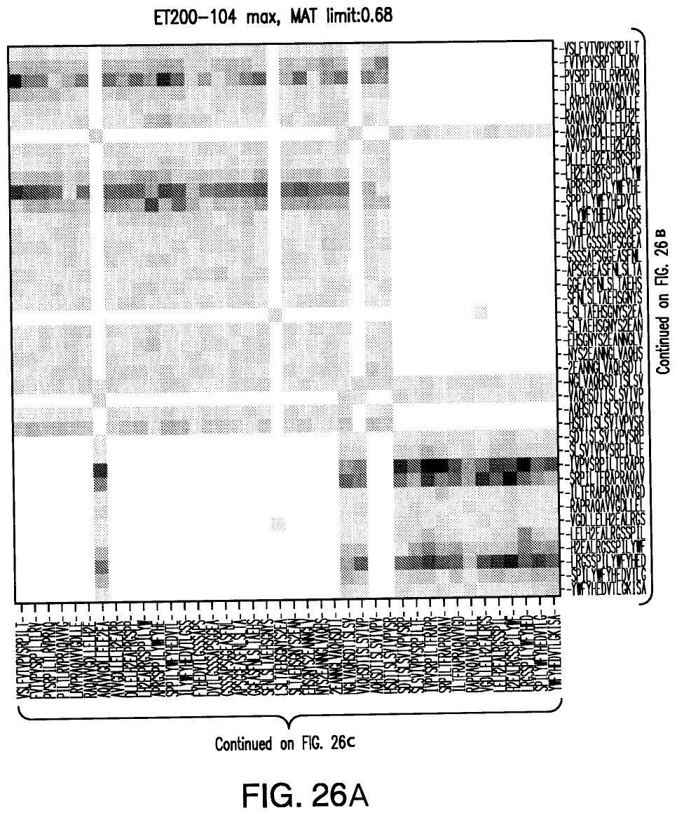

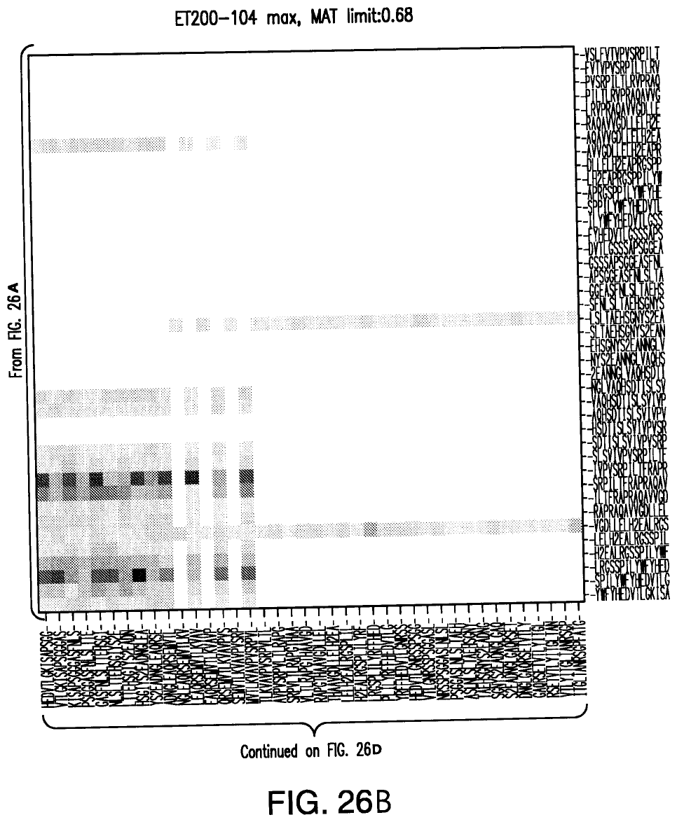

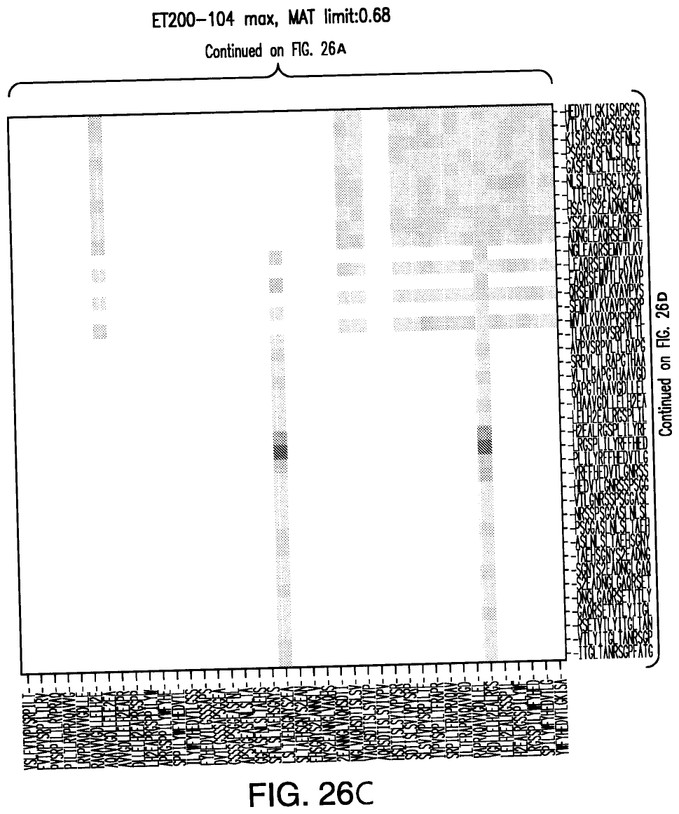

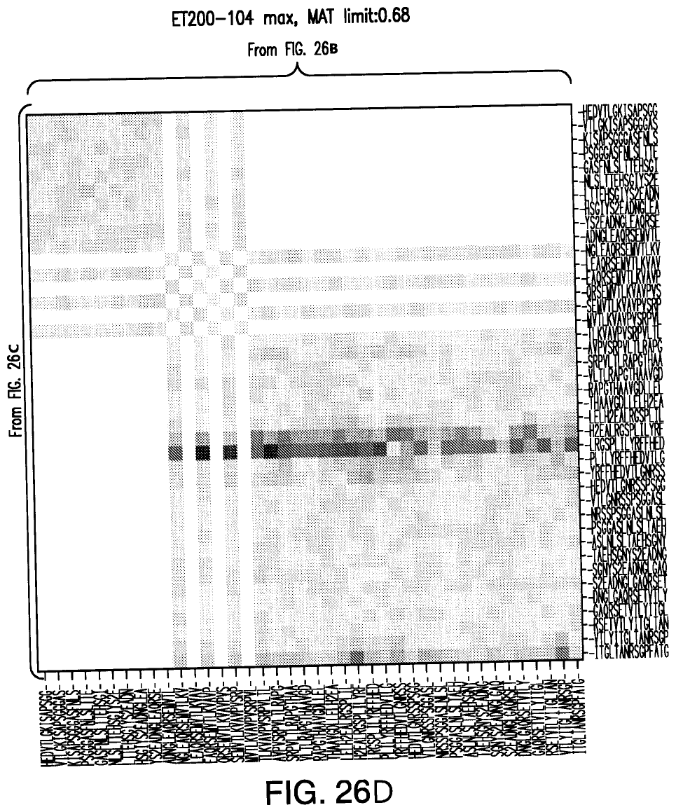

When tested under low stringency conditions and at a high concentration antibody ET200-104 binds multiple peptide motifs in all sets (FIGS. 26A-26D). The majority of peptides bound were suspected to be the result of non-specific hydrophic interactions based on the results obtained for Herceptin (internal negative control). However, binding of peptides containing motif₆₅₇SRPILTFRAPR₆₆₇ was proposed to be specific, and was uniquely attributed to sample ET200-104.

When tested under low stringency conditions antibody ET200-117 resulted in weak binding of multiple peptide motifs on all sets. Cumulative data analysis of data obtained for all sets suggests that the antibody uniquely recognizes a region containing peptide stretch ₈₂₉RSETVTLYITGL₈₄₀ in domain 9 of Fc receptor-like protein 5 distinct from the Herceptin internal negative control and ET200-104. Again the majority of other peptides bound were suspected to be the result of unspecific hydrophic interactions that shared as the same binding pattern was recorded under low stringency conditions for antibody ET200-104.

### CONCLUSIONS

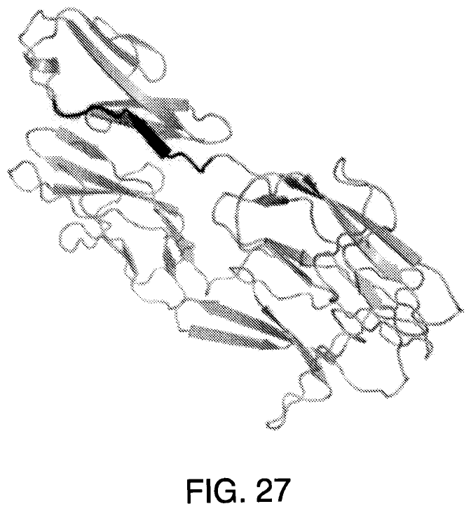

Cumulative data analysis of results collected for ET200-104, and ET200-117 vs. Herceptin suggest that antibody ET200-104 targets residues ₆₅₇SRPILTFRAPR₆₆₇ within domain 8 of Fc receptor-like protein 5 and antibody ET200-117 targets residues ₈₂₉RSETVTLYITGL₈₄₀ within domain 9. Additionally, for both samples multiple signals were recorded with peptides non-specifically bound by Herceptin. The epitope candidate identified for ET200-104 was visualized using a publically available 3D model of Fc receptor-like protein 5 (FIG. 27). The epitope candidate for ET200-117 lies within the non-modeled part of the target and therefore cannot be visualized.

### REFERENCES

1. Franco, A., et al. Human Fc receptor-like 5 binds intact IgG via mechanisms distinct from those of Fc receptors. Journal of immunology 190, 5739-5746 (2013).
2. Dement-Brown, J., et al. Fc receptor-like 5 promotes B cell proliferation and drives the development of cells displaying switched isotypes. Journal of leukocyte biology 91, 59-67 (2012).
3. Ise, T., et al. Elevation of soluble CD307 (IRTA2/FcRH5) protein in the blood and expression on malignant cells of patients with multiple myeloma, chronic lymphocytic leukemia, and mantle cell lymphoma. Leukemia 21, 169-174 (2007).
4. Elkins, K., et al. FcRL5 as a target of antibody-drug conjugates for the treatment of multiple myeloma. Molecular cancer therapeutics 11, 2222-2232 (2012).
5. Hatzivassiliou, G., et al. IRTA1 and IRTA2, novel immunoglobulin superfamily receptors expressed in B cells and involved in chromosome 1q21 abnormalities in B cell malignancy. Immunity 14, 277-289 (2001).
6. An, G., et al. Chromosome 1q21 gains confer inferior outcomes in multiple myeloma treated with bortezomib but copy number variation and percentage of plasma cells involved have no additional prognostic value. Haematologica 99, 353-359 (2014).
7. Ise, T., et al. Immunoglobulin superfamily receptor translocation associated 2 protein on lymphoma cell lines and hairy cell leukemia cells detected by novel monoclonal antibodies. Clinical cancer research: an official journal of the American Association for Cancer Research 11, 87-96 (2005).
8. Ise, T., Kreitman, R.J., Pastan, I. & Nagata, S. Sandwich ELISAs for soluble immunoglobulin superfamily receptor translocation-associated 2 (IRTA2)/FcRH5 (CD307) proteins in human sera. Clinical chemistry and laboratory medicine: CCLM/FESCC 44, 594-602 (2006).
9. Brentjens, R. J., et al. Safety and persistence of adoptively transferred autologous CD19-targeted T cells in patients with relapsed or chemotherapy refractory B-cell leukemias. Blood 118, 4817-4828 (2011).
10. Brentjens, R. J., et al. Eradication of systemic B-cell tumors by genetically targeted human T lymphocytes co-stimulated by CD80 and interleukin-15. Nature medicine 9, 279-286 (2003).
11. Brentjens, R. J., et al. CD19-Targeted T Cells Rapidly Induce Molecular Remissions in Adults with Chemotherapy-Refractory Acute Lymphoblastic Leukemia. Science translational medicine 5, 177ra138 (2013).
12. Davila, M. L., et al. Efficacy and Toxicity Management of 19-28z CAR T Cell Therapy in B Cell Acute Lymphoblastic Leukemia. Science translational medicine 6, 224ra225 (2014).
13. Siegel, R., Naishadham, D. & Jemal, A. Cancer statistics, 2013. CA: a cancer journal for clinicians 63, 11-30 (2013).
14. Boyd, K. D., et al. The clinical impact and molecular biology of del(17p) in multiple myeloma treated with conventional or thalidomide-based therapy. Genes, chromosomes & cancer 50, 765-774 (2011).
15. Shaughnessy, J.D., Jr., et al. A validated gene expression model of high-risk multiple myeloma is defined by deregulated expression of genes mapping to chromosome 1. Blood 109, 2276-2284 (2007).
16. Gahrton, G., et al. Allogeneic bone marrow transplantation in multiple myeloma. European Group for Bone Marrow Transplantation. The New England journal of medicine 325, 1267-1273 (1991).
17. Pegram, H. J., et al. Tumor-targeted T cells modified to secrete IL-12 eradicate systemic tumors without need for prior conditioning. Blood 119, 4133-4141 (2012).
18. Sabrina Bertilaccio, M. T., et al. Low-Dose Lenalidomide Improves CAR-Based Immunotherapy In CLL By Reverting T-Cell Defects In Vivo. Blood 122, 4171 (2013).
19. Bataille, R., et al. The phenotype of normal, reactive and malignant plasma cells. Identification of “many and multiple myelomas” and of new targets for myeloma therapy. Haematologica 91, 1234-1240 (2006).
20. Morgan, R. A., et al. Case report of a serious adverse event following the administration of T cells transduced with a chimeric antigen receptor recognizing ERBB2. Molecular therapy: the journal of the American Society of Gene Therapy 18, 843-851 (2010).
21. Brentjens, R. J., et al. Safety and persistence of adoptively transferred autologous CD19-targeted T cells in patients with relapsed or chemotherapy refractory B-cell leukemias. Blood 118, 4817-4828 (2011).
22. Brentjens, R. J., et al. CD19-targeted T cells rapidly induce molecular remissions in adults with chemotherapy-refractory acute lymphoblastic leukemia. Science translational medicine 5, 177ra138 (2013).
23. Hunder, N. N., et al. Treatment of metastatic melanoma with autologous CD4⁺ T cells against NY-ESO-1. N.Engl.J.Med. 358, 2698-2703 (2008).
24. Rosenberg, S. A., Restifo, N. P., Yang, J. C., Morgan, R.A. & Dudley, M. E. Adoptive cell transfer: a clinical path to effective cancer immunotherapy. Nat.Rev.Cancer 8, 299-308 (2008).
25. Dudley, M.E., et al. Adoptive cell therapy for patients with metastatic melanoma: evaluation of intensive myeloablative chemoradiation preparative regimens. J Clin Oncol 26, 5233-5239 (2008).
26. Brentjens, R. J., et al. Genetically targeted T cells eradicate systemic acute lymphoblastic leukemia xenografts. Clin.Cancer Res. 13, 5426-5435 (2007).
27. Gade, T. P., et al. Targeted elimination of prostate cancer by genetically directed human T lymphocytes. Cancer Res. 65, 9080-9088 (2005).
28. Maher, J., Brentjens, R. J., Gunset, G., Riviere, I. & Sadelain, M. Human T-lymphocyte cytotoxicity and proliferation directed by a single chimeric TCRzeta/CD28 receptor. Nat.Biotechnol. 25 20, 70-75 (2002).
29. Kershaw, M. H., et al. Gene-engineered T cells as a superior adjuvant therapy for metastatic cancer. J Immunol 173, 2143-2150 (2004).
30. Sadelain, M., Brentjens, R. & Riviere, I. The promise and potential pitfalls of chimeric antigen receptors. Curr Opin Immunol (2009).
31. Hollyman, D., et al. Manufacturing validation of biologically functional T cells targeted to CD19 antigen for autologous adoptive cell therapy. JImmunother 32, 169-180 (2009).
32. Sadelain, M., Brentjens, R. & Riviere, I. The basic principles of chimeric antigen receptor design. Cancer discovery 3, 388-398 (2013).
33. Riviere, I., Sadelain, M. & Brentjens, R.J. Novel strategies for cancer therapy: the potential of genetically modified T lymphocytes. Curr Hematol Rep 3, 290-297 (2004).
34. Stephan, M. T., et al. T cell-encoded CD80 and 4-1BBL induce auto- and transco-stimulation, resulting in potent tumor rejection. Nat.Med. 13, 1440-1449 (2007).
35. Krause, A., et al. Antigen-dependent CD28 signaling selectively enhances survival and proliferation in genetically modified activated human primary T lymphocytes. J Exp Med 188, 619-626 (1998).
36. Gong, M. C., et al. Cancer patient T cells genetically targeted to prostate-specific membrane antigen specifically lyse prostate cancer cells and release cytokines in response to prostate-specific membrane antigen. Neoplasia. 1, 123-127 (1999).
37. Lyddane, C., et al. Cutting Edge: CD28 controls dominant regulatory T cell activity during active immunization. J.Immunol. 176, 3306-3310 (2006).

From the foregoing description, it will be apparent that variations and modifications may be made to the invention described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. A chimeric antigen receptor (CAR), comprising an extracellular antigen-binding domain, a transmembrane domain, and an intracellular domain, wherein the extracellular antigen-binding domain specifically binds to Fc Receptor-Like 5 (FcRL5) and comprises:
(ab) a light chain variable region comprising a CDR1, a CDR2, and a CDR3 of the light chain variable region sequence set forth in SEQ ID NO:7, and a heavy chain variable region comprising a CDR1, a CDR2, and a CDR3 of the heavy chain variable region sequence set forth in SEQ ID NO:8;
(ac) a light chain variable region comprising a CDR1, a CDR2, and a CDR3 of the light chain variable region sequence set forth in SEQ ID NO:11, and a heavy chain variable region comprising a CDR1, a CDR2, and a CDR3 of the heavy chain variable region sequence set forth in SEQ ID NO:12;
(ad) a light chain variable region comprising a CDR1, a CDR2, and a CDR3 of the light chain variable region sequence set forth in SEQ ID NO:299, and a heavy chain variable region comprising a CDR1, a CDR2, and a CDR3 of the heavy chain variable region sequence set forth in SEQ ID NO:300;
(ae) a light chain variable region comprising a CDR1, a CDR2, and a CDR3 of the light chain variable region sequence set forth in SEQ ID NO:15, and a heavy chain variable region comprising a CDR1, a CDR2, and a CDR3 of the heavy chain variable region sequence set forth in SEQ ID NO:16;
(af) a light chain variable region comprising a CDR1, a CDR2, and a CDR3 of the light chain variable region sequence set forth in SEQ ID NO:19, and a heavy chain variable region comprising a CDR1, a CDR2, and a CDR3 of the heavy chain variable region sequence set forth in SEQ ID NO:20;
(ag) a light chain variable region comprising a CDR1, a CDR2, and a CDR3 of the light chain variable region sequence set forth in SEQ ID NO:23, and a heavy chain variable region comprising a CDR1, a CDR2, and a CDR3 of the heavy chain variable region sequence set forth in SEQ ID NO:24;
(ah) a light chain variable region comprising a CDR1, a CDR2, and a CDR3 of the light chain variable region sequence set forth in SEQ ID NO:27, and a heavy chain variable region comprising a CDR1, a CDR2, and a CDR3 of the heavy chain variable region sequence set forth in SEQ ID NO:28;
(ai) a light chain variable region comprising a CDR1, a CDR2, and a CDR3 of the light chain variable region sequence set forth in SEQ ID NO:31, and a heavy chain variable region comprising a CDR1, a CDR2, and a CDR3 of the heavy chain variable region sequence set forth in SEQ ID NO:32;
(aj) a light chain variable region comprising a CDR1, a CDR2, and a CDR3 of the light chain variable region sequence set forth in SEQ ID NO:35, and a heavy chain variable region comprising a CDR1, a CDR2, and a CDR3 of the heavy chain variable region sequence set forth in SEQ ID NO:36;
(ak) a light chain variable region comprising a CDR1, a CDR2, and a CDR3 of the light chain variable region sequence set forth in SEQ ID NO:39, and a heavy chain variable region comprising a CDR1, a CDR2, and a CDR3 of the heavy chain variable region sequence set forth in SEQ ID NO:40;
(al) a light chain variable region comprising a CDR1, a CDR2, and a CDR3 of the light chain variable region sequence set forth in SEQ ID NO:43, and a heavy chain variable region comprising a CDR1, a CDR2, and a CDR3 of the heavy chain variable region sequence set forth in SEQ ID NO:44;
(am) a light chain variable region comprising a CDR1, a CDR2, and a CDR3 of the light chain variable region sequence set forth in SEQ ID NO:47, and a heavy chain variable region comprising a CDR1, a CDR2, and a CDR3 of the heavy chain variable region sequence set forth in SEQ ID NO:48;
(an) a light chain variable region comprising a CDR1, a CDR2, and a CDR3 of the light chain variable region sequence set forth in SEQ ID NO:51, and a heavy chain variable region comprising a CDR1, a CDR2, and a CDR3 of the heavy chain variable region sequence set forth in SEQ ID NO:52;
(ao) a light chain variable region comprising a CDR1, a CDR2, and a CDR3 of the light chain variable region sequence set forth in SEQ ID NO:55, and a heavy chain variable region comprising a CDR1, a CDR2, and a CDR3 of the heavy chain variable region sequence set forth in SEQ ID NO:56;
(ap) a light chain variable region comprising a CDR1, a CDR2, and a CDR3 of the light chain variable region sequence set forth in SEQ ID NO:59, and a heavy chain variable region comprising a CDR1, a CDR2, and a CDR3 of the heavy chain variable region sequence set forth in SEQ ID NO:60;
(aq) a light chain variable region comprising a CDR1, a CDR2, and a CDR3 of the light chain variable region sequence set forth in SEQ ID NO:63, and a heavy chain variable region comprising a CDR1, a CDR2, and a CDR3 of the heavy chain variable region sequence set forth in SEQ ID NO:64;
(ar) a light chain variable region comprising a CDR1, a CDR2, and a CDR3 of the light chain variable region sequence set forth in SEQ ID NO:67, and a heavy chain variable region comprising a CDR1, a CDR2, and a CDR3 of the heavy chain variable region sequence set forth in SEQ ID NO:68;
(as) a light chain variable region comprising a CDR1, a CDR2, and a CDR3 of the light chain variable region sequence set forth in SEQ ID NO:71, and a heavy chain variable region comprising a CDR1, a CDR2, and a CDR3 of the heavy chain variable region sequence set forth in SEQ ID NO:72;
(at) a light chain variable region comprising a CDR1, a CDR2, and a CDR3 of the light chain variable region sequence set forth in SEQ ID NO:75, and a heavy chain variable region comprising a CDR1, a CDR2, and a CDR3 of the heavy chain variable region sequence set forth in SEQ ID NO:76;
(au) a light chain variable region comprising a CDR1, a CDR2, and a CDR3 of the light chain variable region sequence set forth in SEQ ID NO:79, and a heavy chain variable region comprising a CDR1, a CDR2, and a CDR3 of the heavy chain variable region sequence set forth in SEQ ID NO:80;
(av) a light chain variable region comprising a CDR1, a CDR2, and a CDR3 of the light chain variable region sequence set forth in SEQ ID NO:83, and a heavy chain variable region comprising a CDR1, a CDR2, and a CDR3 of the heavy chain variable region sequence set forth in SEQ ID NO:84;
(aw) a light chain variable region comprising a CDR1, a CDR2, and a CDR3 of the light chain variable region sequence set forth in SEQ ID NO:87, and a heavy chain variable region comprising a CDR1, a CDR2, and a CDR3 of the heavy chain variable region sequence set forth in SEQ ID NO:88;
(ax) a light chain variable region comprising a CDR1, a CDR2, and a CDR3 of the light chain variable region sequence set forth in SEQ ID NO:91, and a heavy chain variable region comprising a CDR1, a CDR2, and a CDR3 of the heavy chain variable region sequence set forth in SEQ ID NO:92;
(ay) a light chain variable region comprising a CDR1, a CDR2, and a CDR3 of the light chain variable region sequence set forth in SEQ ID NO:95, and a heavy chain variable region comprising a CDR1, a CDR2, and a CDR3 of the heavy chain variable region sequence set forth in SEQ ID NO:96;
(az) a light chain variable region comprising a CDR1, a CDR2, and a CDR3 of the light chain variable region sequence set forth in SEQ ID NO:99, and a heavy chain variable region comprising a CDR1, a CDR2, and a CDR3 of the heavy chain variable region sequence set forth in SEQ ID NO:100;
(ba) a light chain variable region comprising a CDR1, a CDR2, and a CDR3 of the light chain variable region sequence set forth in SEQ ID NO:103, and a heavy chain variable region comprising a CDR1, a CDR2, and a CDR3 of the heavy chain variable region sequence set forth in SEQ ID NO:104;
(bb) a light chain variable region comprising a CDR1, a CDR2, and a CDR3 of the light chain variable region sequence set forth in SEQ ID NO:107, and a heavy chain variable region comprising a CDR1, a CDR2, and a CDR3 of the heavy chain variable region sequence set forth in SEQ ID NO:108;
(bc) a light chain variable region comprising a CDR1, a CDR2, and a CDR3 of the light chain variable region sequence set forth in SEQ ID NO:111, and a heavy chain variable region comprising a CDR1, a CDR2, and a CDR3 of the heavy chain variable region sequence set forth in SEQ ID NO:112;
(be) a light chain variable region comprising a CDR1, a CDR2, and a CDR3 of the light chain variable region sequence set forth in SEQ ID NO:119, and a heavy chain variable region comprising a CDR1, a CDR2, and a CDR3 of the heavy chain variable region sequence set forth in SEQ ID NO:120;
(bf) a light chain variable region comprising a CDR1, a CDR2, and a CDR3 of the light chain variable region sequence set forth in SEQ ID NO:123, and a heavy chain variable region comprising a CDR1, a CDR2, and a CDR3 of the heavy chain variable region sequence set forth in SEQ ID NO:124;
(bg) a light chain variable region comprising a CDR1, a CDR2, and a CDR3 of the light chain variable region sequence set forth in SEQ ID NO:127, and a heavy chain variable region comprising a CDR1, a CDR2, and a CDR3 of the heavy chain variable region sequence set forth in SEQ ID NO:128;
(bh) a light chain variable region comprising a CDR1, a CDR2, and a CDR3 of the light chain variable region sequence set forth in SEQ ID NO:131, and a heavy chain variable region comprising a CDR1, a CDR2, and a CDR3 of the heavy chain variable region sequence set forth in SEQ ID NO:132;
(bi) a light chain variable region comprising a CDR1, a CDR2, and a CDR3 of the light chain variable region sequence set forth in SEQ ID NO:135, and a heavy chain variable region comprising a CDR1, a CDR2, and a CDR3 of the heavy chain variable region sequence set forth in SEQ ID NO:136;
(bj) a light chain variable region comprising a CDR1, a CDR2, and a CDR3 of the light chain variable region sequence set forth in SEQ ID NO:139, and a heavy chain variable region comprising a CDR1, a CDR2, and a CDR3 of the heavy chain variable region sequence set forth in SEQ ID NO:140;
(bl) a light chain variable region comprising a CDR1, a CDR2, and a CDR3 of the light chain variable region sequence set forth in SEQ ID NO:147, and a heavy chain variable region comprising a CDR1, a CDR2, and a CDR3 of the heavy chain variable region sequence set forth in SEQ ID NO:148;
(bm) a light chain variable region comprising a CDR1, a CDR2, and a CDR3 of the light chain variable region sequence set forth in SEQ ID NO:151, and a heavy chain variable region comprising a CDR1, a CDR2, and a CDR3 of the heavy chain variable region sequence set forth in SEQ ID NO:152;
(bn) a light chain variable region comprising a CDR1, a CDR2, and a CDR3 of the light chain variable region sequence set forth in SEQ ID NO:155, and a heavy chain variable region comprising a CDR1, a CDR2, and a CDR3 of the heavy chain variable region sequence set forth in SEQ ID NO:156;
(bo) a light chain variable region comprising a CDR1, a CDR2, and a CDR3 of the light chain variable region sequence set forth in SEQ ID NO:159, and a heavy chain variable region comprising a CDR1, a CDR2, and a CDR3 of the heavy chain variable region sequence set forth in SEQ ID NO:160;
(bp) a light chain variable region comprising a CDR1, a CDR2, and a CDR3 of the light chain variable region sequence set forth in SEQ ID NO:163, and a heavy chain variable region comprising a CDR1, a CDR2, and a CDR3 of the heavy chain variable region sequence set forth in SEQ ID NO:164;
(bq) a light chain variable region comprising a CDR1, a CDR2, and a CDR3 of the light chain variable region sequence set forth in SEQ ID NO:167, and a heavy chain variable region comprising a CDR1, a CDR2, and a CDR3 of the heavy chain variable region sequence set forth in SEQ ID NO:168;
(bs) a light chain variable region comprising a CDR1, a CDR2, and a CDR3 of the light chain variable region sequence set forth in SEQ ID NO:175, and a heavy chain variable region comprising a CDR1, a CDR2, and a CDR3 of the heavy chain variable region sequence set forth in SEQ ID NO:176;
(bt) a light chain variable region comprising a CDR1, a CDR2, and a CDR3 of the light chain variable region sequence set forth in SEQ ID NO:179, and a heavy chain variable region comprising a CDR1, a CDR2, and a CDR3 of the heavy chain variable region sequence set forth in SEQ ID NO:180;
(bu) a light chain variable region comprising a CDR1, a CDR2, and a CDR3 of the light chain variable region sequence set forth in SEQ ID NO:183, and a heavy chain variable region comprising a CDR1, a CDR2, and a CDR3 of the heavy chain variable region sequence set forth in SEQ ID NO:184;
(by) a light chain variable region comprising a CDR1, a CDR2, and a CDR3 of the light chain variable region sequence set forth in SEQ ID NO:187, and a heavy chain variable region comprising a CDR1, a CDR2, and a CDR3 of the heavy chain variable region sequence set forth in SEQ ID NO:188;
(bw) a light chain variable region comprising a CDR1, a CDR2, and a CDR3 of the light chain variable region sequence set forth in SEQ ID NO:191, and a heavy chain variable region comprising a CDR1, a CDR2, and a CDR3 of the heavy chain variable region sequence set forth in SEQ ID NO:192;
(bx) a light chain variable region comprising a CDR1, a CDR2, and a CDR3 of the light chain variable region sequence set forth in SEQ ID NO:195, and a heavy chain variable region comprising a CDR1, a CDR2, and a CDR3 of the heavy chain variable region sequence set forth in SEQ ID NO:196;
(by) a light chain variable region comprising a CDR1, a CDR2, and a CDR3 of the light chain variable region sequence set forth in SEQ ID NO:199, and a heavy chain variable region comprising a CDR1, a CDR2, and a CDR3 of the heavy chain variable region sequence set forth in SEQ ID NO:200;
(bz) a light chain variable region comprising a CDR1, a CDR2, and a CDR3 of the light chain variable region sequence set forth in SEQ ID NO:203, and a heavy chain variable region comprising a CDR1, a CDR2, and a CDR3 of the heavy chain variable region sequence set forth in SEQ ID NO:204;
(ca) a light chain variable region comprising a CDR1, a CDR2, and a CDR3 of the light chain variable region sequence set forth in SEQ ID NO:207, and a heavy chain variable region comprising a CDR1, a CDR2, and a CDR3 of the heavy chain variable region sequence set forth in SEQ ID NO:208;
(cb) a light chain variable region comprising a CDR1, a CDR2, and a CDR3 of the light chain variable region sequence set forth in SEQ ID NO:211, and a heavy chain variable region comprising a CDR1, a CDR2, and a CDR3 of the heavy chain variable region sequence set forth in SEQ ID NO:212;
(ce) a light chain variable region comprising a CDR1, a CDR2, and a CDR3 of the light chain variable region sequence set forth in SEQ ID NO:223, and a heavy chain variable region comprising a CDR1, a CDR2, and a CDR3 of the heavy chain variable region sequence set forth in SEQ ID NO:224;
(cf) a light chain variable region comprising a CDR1, a CDR2, and a CDR3 of the light chain variable region sequence set forth in SEQ ID NO:227, and a heavy chain variable region comprising a CDR1, a CDR2, and a CDR3 of the heavy chain variable region sequence set forth in SEQ ID NO:228;
(cg) a light chain variable region comprising a CDR1, a CDR2, and a CDR3 of the light chain variable region sequence set forth in SEQ ID NO:231, and a heavy chain variable region comprising a CDR1, a CDR2, and a CDR3 of the heavy chain variable region sequence set forth in SEQ ID NO:232;
(ci) a light chain variable region comprising a CDR1, a CDR2, and a CDR3 of the light chain variable region sequence set forth in SEQ ID NO:239, and a heavy chain variable region comprising a CDR1, a CDR2, and a CDR3 of the heavy chain variable region sequence set forth in SEQ ID NO:240;
(cj) a light chain variable region comprising a CDR1, a CDR2, and a CDR3 of the light chain variable region sequence set forth in SEQ ID NO:243, and a heavy chain variable region comprising a CDR1, a CDR2, and a CDR3 of the heavy chain variable region sequence set forth in SEQ ID NO:244;
(ck) a light chain variable region comprising a CDR1, a CDR2, and a CDR3 of the light chain variable region sequence set forth in SEQ ID NO:247, and a heavy chain variable region comprising a CDR1, a CDR2, and a CDR3 of the heavy chain variable region sequence set forth in SEQ ID NO:248;
(cl) a light chain variable region comprising a CDR1, a CDR2, and a CDR3 of the light chain variable region sequence set forth in SEQ ID NO:251, and a heavy chain variable region comprising a CDR1, a CDR2, and a CDR3 of the heavy chain variable region sequence set forth in SEQ ID NO:252;
(cm) a light chain variable region comprising a CDR1, a CDR2, and a CDR3 of the light chain variable region sequence set forth in SEQ ID NO:255, and a heavy chain variable region comprising a CDR1, a CDR2, and a CDR3 of the heavy chain variable region sequence set forth in SEQ ID NO:256;
(cn) a light chain variable region comprising a CDR1, a CDR2, and a CDR3 of the light chain variable region sequence set forth in SEQ ID NO:259, and a heavy chain variable region comprising a CDR1, a CDR2, and a CDR3 of the heavy chain variable region sequence set forth in SEQ ID NO:260;
(co) a light chain variable region comprising a CDR1, a CDR2, and a CDR3 of the light chain variable region sequence set forth in SEQ ID NO:263, and a heavy chain variable region comprising a CDR1, a CDR2, and a CDR3 of the heavy chain variable region sequence set forth in SEQ ID NO:264;
(cq) a light chain variable region comprising a CDR1, a CDR2, and a CDR3 of the light chain variable region sequence set forth in SEQ ID NO:271, and a heavy chain variable region comprising a CDR1, a CDR2, and a CDR3 of the heavy chain variable region sequence set forth in SEQ ID NO:272;
(cr) a light chain variable region comprising a CDR1, a CDR2, and a CDR3 of the light chain variable region sequence set forth in SEQ ID NO:275, and a heavy chain variable region comprising a CDR1, a CDR2, and a CDR3 of the heavy chain variable region sequence set forth in SEQ ID NO:276;
(cs) a light chain variable region comprising a CDR1, a CDR2, and a CDR3 of the light chain variable region sequence set forth in SEQ ID NO:279, and a heavy chain variable region comprising a CDR1, a CDR2, and a CDR3 of the heavy chain variable region sequence set forth in SEQ ID NO:280;
(ct) a light chain variable region comprising a CDR1, a CDR2, and a CDR3 of the light chain variable region sequence set forth in SEQ ID NO:283, and a heavy chain variable region comprising a CDR1, a CDR2, and a CDR3 of the heavy chain variable region sequence set forth in SEQ ID NO:284;
(cu) a light chain variable region comprising a CDR1, a CDR2, and a CDR3 of the light chain variable region sequence set forth in SEQ ID NO:287, and a heavy chain variable region comprising a CDR1, a CDR2, and a CDR3 of the heavy chain variable region sequence set forth in SEQ ID NO:288;
(cv) a light chain variable region comprising a CDR1, a CDR2, and a CDR3 of the light chain variable region sequence set forth in SEQ ID NO:291, and a heavy chain variable region comprising a CDR1, a CDR2, and a CDR3 of the heavy chain variable region sequence set forth in SEQ ID NO:292;
(cw) a light chain variable region comprising a CDR1, a CDR2, and a CDR3 of the light chain variable region sequence set forth in SEQ ID NO:303, and a heavy chain variable region comprising a CDR1, a CDR2, and a CDR3 of the heavy chain variable region sequence set forth in SEQ ID NO:304;
(cx) a light chain variable region comprising a CDR1, a CDR2, and a CDR3 of the light chain variable region sequence set forth in SEQ ID NO:295, and a heavy chain variable region comprising a CDR1, a CDR2, and a CDR3 of the heavy chain variable region sequence set forth in SEQ ID NO:296;
(cy) a light chain variable region comprising a CDR1, a CDR2, and a CDR3 of the light chain variable region sequence set forth in SEQ ID NO:917, and a heavy chain variable region comprising a CDR1, a CDR2, and a CDR3 of the heavy chain variable region sequence set forth in SEQ ID NO:915; or
(cz) a light chain variable region comprising a CDR1, a CDR2, and a CDR3 of the light chain variable region sequence set forth in SEQ ID NO:921, and a heavy chain variable region comprising a CDR1, a CDR2, and a CDR3 of the heavy chain variable region sequence set forth in SEQ ID NO:919.

2. The CAR of claim 1, wherein:
(ab) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:320; and the heavy chain variable region comprises a CDR1 comprising the amino acid sequence set forth in SEQ ID NO:315, a CDR2 comprising the amino acid sequence set forth in SEQ ID NO:316, and a CDR3 comprising the amino acid sequence set forth in SEQ ID NO:317;
(ac) the light chain variable region comprises a CDR1 comprising the amino acid sequence set forth in SEQ ID NO:324, a CDR2 comprising the amino acid sequence set forth in SEQ ID NO:319, and a CDR3 comprising the amino acid sequence set forth in SEQ ID NO:325; and the heavy chain variable region comprises a CDR1 comprising the amino acid sequence set forth in SEQ ID NO:321, a CDR2 comprising the amino acid sequence set forth in SEQ ID NO:322, and a CDR3 comprising the amino acid sequence set forth in SEQ ID NO:323;
(ad) the light chain variable region comprises a CDR1 comprising the amino acid sequence set forth in SEQ ID NO:329, a CDR2 comprising the amino acid sequence set forth in SEQ ID NO:330, and a CDR3 comprising the amino acid sequence set forth in SEQ ID NO:331; and the heavy chain variable region comprises a CDR1 comprising the amino acid sequence set forth in SEQ ID NO:326, a CDR2 comprising the amino acid sequence set forth in SEQ ID NO:327, and a CDR3 comprising the amino acid sequence set forth in SEQ ID NO:328;
(ae) the light chain variable region comprises a CDR1 comprising the amino acid sequence set forth in SEQ ID NO:329, a CDR2 comprising the amino acid sequence set forth in SEQ ID NO:330, and a CDR3 comprising the amino acid sequence set forth in SEQ ID NO:331; and the heavy chain variable region comprises a CDR1 comprising the amino acid sequence set forth in SEQ ID NO:332, a CDR2 comprising the amino acid sequence set forth in SEQ ID NO:333, and a CDR3 comprising the amino acid sequence set forth in SEQ ID NO:334;
(af) the light chain variable region comprises a CDR1 comprising the amino acid sequence set forth in SEQ ID NO:338, a CDR2 comprising the amino acid sequence set forth in SEQ ID NO:330, and a CDR3 comprising the amino acid sequence set forth in SEQ ID NO:339; and the heavy chain variable region comprises a CDR1 comprising the amino acid sequence set forth in SEQ ID NO:335, a CDR2 comprising the amino acid sequence set forth in SEQ ID NO:336, and a CDR3 comprising the amino acid sequence set forth in SEQ ID NO:337;
(ag) the light chain variable region comprises a CDR1 comprising the amino acid sequence set forth in SEQ ID NO:343, a CDR2 comprising the amino acid sequence set forth in SEQ ID NO:344, and a CDR3 comprising the amino acid sequence set forth in SEQ ID NO:345; and the heavy chain variable region comprises a CDR1 comprising the amino acid sequence set forth in SEQ ID NO:340, a CDR2 comprising the amino acid sequence set forth in SEQ ID NO:341, and a CDR3 comprising the amino acid sequence set forth in SEQ ID NO:342;
(ah) the light chain variable region comprises a CDR1 comprising the amino acid sequence set forth in SEQ ID NO:348, a CDR2 comprising the amino acid sequence set forth in SEQ ID NO:349, and a CDR3 comprising the amino acid sequence set forth in SEQ ID NO:350; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:347;
(ai) the light chain variable region comprises a CDR1 comprising the amino acid sequence set forth in SEQ ID NO:352, a CDR2 comprising the amino acid sequence set forth in SEQ ID NO:344, and a CDR3 comprising the amino acid sequence set forth in SEQ ID NO:353; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:351;
(aj) the light chain variable region comprises a CDR1 comprising the amino acid sequence set forth in SEQ ID NO:357, a CDR2 comprising the amino acid sequence set forth in SEQ ID NO:358, and a CDR3 comprising the amino acid sequence set forth in SEQ ID NO:359; and the heavy chain variable region comprises a CDR1 comprising the amino acid sequence set forth in SEQ ID NO:354, a CDR2 comprising the amino acid sequence set forth in SEQ ID NO:355, and a CDR3 comprising the amino acid sequence set forth in SEQ ID NO:356;
(ak) the light chain variable region comprises a CDR1 comprising the amino acid sequence set forth in SEQ ID NO:363, a CDR2 comprising the amino acid sequence set forth in SEQ ID NO:364, and a CDR3 comprising the amino acid sequence set forth in SEQ ID NO:365; and the heavy chain variable region comprises a CDR1 comprising the amino acid sequence set forth in SEQ ID NO:360, a CDR2 comprising the amino acid sequence set forth in SEQ ID NO:361, and a CDR3 comprising the amino acid sequence set forth in SEQ ID NO:362;
(al) the light chain variable region comprises a CDR1 comprising the amino acid sequence set forth in SEQ ID NO:369, a CDR2 comprising the amino acid sequence set forth in SEQ ID NO:370, and a CDR3 comprising the amino acid sequence set forth in SEQ ID NO:371; and the heavy chain variable region comprises a CDR1 comprising the amino acid sequence set forth in SEQ ID NO:366, a CDR2 comprising the amino acid sequence set forth in SEQ ID NO:367, and a CDR3 comprising the amino acid sequence set forth in SEQ ID NO:368;
(am) the light chain variable region comprises a CDR1 comprising the amino acid sequence set forth in SEQ ID NO:329, a CDR2 comprising the amino acid sequence set forth in SEQ ID NO:330, and a CDR3 comprising the amino acid sequence set forth in SEQ ID NO:375; and the heavy chain variable region comprisesa CDR1 comprising the amino acid sequence set forth in SEQ ID NO:372, a CDR2 comprising the amino acid sequence set forth in SEQ ID NO:373, and a CDR3 comprising the amino acid sequence set forth in SEQ ID NO:374;
(an) the light chain variable region comprises a CDR1 comprising the amino acid sequence set forth in SEQ ID NO:329, a CDR2 comprising the amino acid sequence set forth in SEQ ID NO:330, and a CDR3 comprising the amino acid sequence set forth in SEQ ID NO:377; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:376;
(ao) the light chain variable region comprises a CDR1 comprising the amino acid sequence set forth in SEQ ID NO:381, a CDR2 comprising the amino acid sequence set forth in SEQ ID NO:382, and a CDR3 comprising the amino acid sequence set forth in SEQ ID NO:383; and the heavy chain variable region comprises a CDR1 comprising the amino acid sequence set forth in SEQ ID NO:378, a CDR2 comprising the amino acid sequence set forth in SEQ ID NO:379, and a CDR3 comprising the amino acid sequence set forth in SEQ ID NO:380;
(ap) the light chain variable region comprises a CDR1 comprising the amino acid sequence set forth in SEQ ID NO:329, a CDR2 comprising the amino acid sequence set forth in SEQ ID NO:385, and a CDR3 comprising the amino acid sequence set forth in SEQ ID NO:386; and the heavy chain variable region comprises a CDR1 comprising the amino acid sequence set forth in SEQ ID NO:309, a CDR2 comprising the amino acid sequence set forth in SEQ ID NO:310, and a CDR3 comprising the amino acid sequence set forth in SEQ ID NO:384;
(aq) the light chain variable region comprises a CDR1 comprising the amino acid sequence set forth in SEQ ID NO:390, a CDR2 comprising the amino acid sequence set forth in SEQ ID NO:391, and a CDR3 comprising the amino acid sequence set forth in SEQ ID NO:392; and the heavy chain variable region comprises a CDR1 comprising the amino acid sequence set forth in SEQ ID NO:387, a CDR2 comprising the amino acid sequence set forth in SEQ ID NO:388, and a CDR3 comprising the amino acid sequence set forth in SEQ ID NO:389;
(ar) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:395; and the heavy chain variable region comprises a CDR1 comprising the amino acid sequence set forth in SEQ ID NO:393, a CDR2 comprising the amino acid sequence set forth in SEQ ID NO:355, and a CDR3 comprising the amino acid sequence set forth in SEQ ID NO:394;
(as) the light chain variable region comprises a CDR1 comprising the amino acid sequence set forth in SEQ ID NO:397, a CDR2 comprising the amino acid sequence set forth in SEQ ID NO:398, and a CDR3 comprising the amino acid sequence set forth in SEQ ID NO:399; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:396;
(at) the light chain variable region comprises a CDR1 comprising the amino acid sequence set forth in SEQ ID NO:401, a CDR2 comprising the amino acid sequence set forth in SEQ ID NO:398, and a CDR3 comprising the amino acid sequence set forth in SEQ ID NO:402; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:400;
(au) the light chain variable region comprises a CDR1 comprising the amino acid sequence set forth in SEQ ID NO:406, a CDR2 comprising the amino acid sequence set forth in SEQ ID NO:398, and a CDR3 comprising the amino acid sequence set forth in SEQ ID NO:407; and the heavy chain variable region comprises a CDR1 comprising the amino acid sequence set forth in SEQ ID NO:403, a CDR2 comprising the amino acid sequence set forth in SEQ ID NO:404, and a CDR3 comprising the amino acid sequence set forth in SEQ ID NO:405;
(av) the light chain variable region comprises a CDR1 comprising the amino acid sequence set forth in SEQ ID NO:329, a CDR2 comprising the amino acid sequence set forth in SEQ ID NO:409, and a CDR3 comprising the amino acid sequence set forth in SEQ ID NO:410; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:408;
(aw) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:414; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:413;
(ax) the light chain variable region comprises a CDR1 comprising the amino acid sequence set forth in SEQ ID NO:416, a CDR2 comprising the amino acid sequence set forth in SEQ ID NO:417, and a CDR3 comprising the amino acid sequence set forth in SEQ ID NO:418; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:415;
(ay) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:419; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:349;
(az) the light chain variable region comprises a CDR1 comprising the amino acid sequence set forth in SEQ ID NO:423, a CDR2 comprising the amino acid sequence set forth in SEQ ID NO:358, and a CDR3 comprising the amino acid sequence set forth in SEQ ID NO:424; and the heavy chain variable region comprises a CDR1 comprising the amino acid sequence set forth in SEQ ID NO:420, a CDR2 comprising the amino acid sequence set forth in SEQ ID NO:421, and a CDR3 comprising the amino acid sequence set forth in SEQ ID NO:422;
(ba) the light chain variable region comprises a CDR1 comprising the amino acid sequence set forth in SEQ ID NO:428, a CDR2 comprising the amino acid sequence set forth in SEQ ID NO:429, and a CDR3 comprising the amino acid sequence set forth in SEQ ID NO:430; and the heavy chain variable region comprises a CDR1 comprising the amino acid sequence set forth in SEQ ID NO:425, a CDR2 comprising the amino acid sequence set forth in SEQ ID NO:426, and a CDR3 comprising the amino acid sequence set forth in SEQ ID NO:427;
(bb) the light chain variable region comprises a CDR1 comprising the amino acid sequence set forth in SEQ ID NO:433, a CDR2 comprising the amino acid sequence set forth in SEQ ID NO:434, and a CDR3 comprising the amino acid sequence set forth in SEQ ID NO:435; and the heavy chain variable region comprises a CDR1 comprising the amino acid sequence set forth in SEQ ID NO:372, a CDR2 comprising the amino acid sequence set forth in SEQ ID NO:431, and a CDR3 comprising the amino acid sequence set forth in SEQ ID NO:432;
(bc) the light chain variable region comprises a CDR1 comprising the amino acid sequence set forth in SEQ ID NO:423, a CDR2 comprising the amino acid sequence set forth in SEQ ID NO:438, and a CDR3 comprising the amino acid sequence set forth in SEQ ID NO:439; and the heavy chain variable region comprises a CDR1 comprising the amino acid sequence set forth in SEQ ID NO:436, a CDR2 comprising the amino acid sequence set forth in SEQ ID NO:388, and a CDR3 comprising the amino acid sequence set forth in SEQ ID NO:437;
(be) the light chain variable region comprises a CDR1 comprising the amino acid sequence set forth in SEQ ID NO:447, a CDR2 comprising the amino acid sequence set forth in SEQ ID NO:448, and a CDR3 comprising the amino acid sequence set forth in SEQ ID NO:449; and the heavy chain variable region comprises a CDR1 comprising the amino acid sequence set forth in SEQ ID NO:444, a CDR2 comprising the amino acid sequence set forth in SEQ ID NO:445, and a CDR3 comprising the amino acid sequence set forth in SEQ ID NO:446;
(bf) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:452; and the heavy chain variable region comprises a CDR1 comprising the amino acid sequence set forth in SEQ ID NO:309, a CDR2 comprising the amino acid sequence set forth in SEQ ID NO:450, and a CDR3 comprising the amino acid sequence set forth in SEQ ID NO:451;
(bg) the light chain variable region comprises a CDR1 comprising the amino acid sequence set forth in SEQ ID NO:369, a CDR2 comprising the amino acid sequence set forth in SEQ ID NO:454, and a CDR3 comprising the amino acid sequence set forth in SEQ ID NO:455; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:453;
(bh) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:457; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:456;
(bi) the light chain variable region comprises a CDR1 comprising the amino acid sequence set forth in SEQ ID NO:329, a CDR2 comprising the amino acid sequence set forth in SEQ ID NO:330, and a CDR3 comprising the amino acid sequence set forth in SEQ ID NO:331; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:458;
(bj) the light chain variable region comprises a CDR1 comprising the amino acid sequence set forth in SEQ ID NO:460, a CDR2 comprising the amino acid sequence set forth in SEQ ID NO:461, and a CDR3 comprising the amino acid sequence set forth in SEQ ID NO:462; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:459;
(bl) the light chain variable region comprises a CDR1 comprising the amino acid sequence set forth in SEQ ID NO:423, a CDR2 comprising the amino acid sequence set forth in SEQ ID NO:438, and a CDR3 comprising the amino acid sequence set forth in SEQ ID NO:465; and the heavy chain variable region comprises a CDR1 comprising the amino acid sequence set forth in SEQ ID NO:436, a CDR2 comprising the amino acid sequence set forth in SEQ ID NO:388, and a CDR3 comprising the amino acid sequence set forth in SEQ ID NO:464;
(bm) the light chain variable region comprises a CDR1 comprising the amino acid sequence set forth in SEQ ID NO:468, a CDR2 comprising the amino acid sequence set forth in SEQ ID NO:469, and a CDR3 comprising the amino acid sequence set forth in SEQ ID NO:470; and the heavy chain variable region comprises a CDR1 comprising the amino acid sequence set forth in SEQ ID NO:411, a CDR2 comprising the amino acid sequence set forth in SEQ ID NO:466, and a CDR3 comprising the amino acid sequence set forth in SEQ ID NO:467;
(bn) the light chain variable region comprises a CDR1 comprising the amino acid sequence set forth in SEQ ID NO:474, a CDR2 comprising the amino acid sequence set forth in SEQ ID NO:358, and a CDR3 comprising the amino acid sequence set forth in SEQ ID NO:465; and the heavy chain variable region comprises a CDR1 comprising the amino acid sequence set forth in SEQ ID NO:471, a CDR2 comprising the amino acid sequence set forth in SEQ ID NO:472, and a CDR3 comprising the amino acid sequence set forth in SEQ ID NO:473;
(bo) the light chain variable region comprises a CDR1 comprising the amino acid sequence set forth in SEQ ID NO:477, a CDR2 comprising the amino acid sequence set forth in SEQ ID NO:478, and a CDR3 comprising the amino acid sequence set forth in SEQ ID NO:479; and the heavy chain variable region comprises a CDR1 comprising the amino acid sequence set forth in SEQ ID NO:372, a CDR2 comprising the amino acid sequence set forth in SEQ ID NO:475, and a CDR3 comprising the amino acid sequence set forth in SEQ ID NO:476;
(bp) the light chain variable region comprises a CDR1 comprising the amino acid sequence set forth in SEQ ID NO:483, a CDR2 comprising the amino acid sequence set forth in SEQ ID NO:484, and a CDR3 comprising the amino acid sequence set forth in SEQ ID NO:485; and the heavy chain variable region comprises a CDR1 comprising the amino acid sequence set forth in SEQ ID NO:480, a CDR2 comprising the amino acid sequence set forth in SEQ ID NO:481, and a CDR3 comprising the amino acid sequence set forth in SEQ ID NO:482;
(bq) the light chain variable region comprises a CDR1 comprising the amino acid sequence set forth in SEQ ID NO:433, a CDR2 comprising the amino acid sequence set forth in SEQ ID NO:487, and a CDR3 comprising the amino acid sequence set forth in SEQ ID NO:488; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:486;
(bs) the light chain variable region comprises a CDR1 comprising the amino acid sequence set forth in SEQ ID NO:312, a CDR2 comprising the amino acid sequence set forth in SEQ ID NO:313, and a CDR3 comprising the amino acid sequence set forth in SEQ ID NO:493; and the heavy chain variable region comprises a CDR1 comprising the amino acid sequence set forth in SEQ ID NO:309, a CDR2 comprising the amino acid sequence set forth in SEQ ID NO:310, and a CDR3 comprising the amino acid sequence set forth in SEQ ID NO:492;
(bt) the light chain variable region comprises a CDR1 comprising the amino acid sequence set forth in SEQ ID NO:490, a CDR2 comprising the amino acid sequence set forth in SEQ ID NO:349, and a CDR3 comprising the amino acid sequence set forth in SEQ ID NO:495; and the heavy chain variable region comprises a CDR1 comprising the amino acid sequence set forth in SEQ ID NO:403, a CDR2 comprising the amino acid sequence set forth in SEQ ID NO:404, and a CDR3 comprising the amino acid sequence set forth in SEQ ID NO:494;
(bu) the light chain variable region comprises a CDR1 comprising the amino acid sequence set forth in SEQ ID NO:498, a CDR2 comprising the amino acid sequence set forth in SEQ ID NO:344, and a CDR3 comprising the amino acid sequence set forth in SEQ ID NO:499; and the heavy chain variable region comprises a CDR1 comprising the amino acid sequence set forth in SEQ ID NO:403, a CDR2 comprising the amino acid sequence set forth in SEQ ID NO:496, and a CDR3 comprising the amino acid sequence set forth in SEQ ID NO:497;
(by) the light chain variable region comprises a CDR1 comprising the amino acid sequence set forth in SEQ ID NO:503, a CDR2 comprising the amino acid sequence set forth in SEQ ID NO:504, and a CDR3 comprising the amino acid sequence set forth in SEQ ID NO:505; and the heavy chain variable region comprises a CDR1 comprising the amino acid sequence set forth in SEQ ID NO:500, a CDR2 comprising the amino acid sequence set forth in SEQ ID NO:501, and a CDR3 comprising the amino acid sequence set forth in SEQ ID NO:502;
(bw) the light chain variable region comprises a CDR1 comprising the amino acid sequence set forth in SEQ ID NO:508, a CDR2 comprising the amino acid sequence set forth in SEQ ID NO:349, and a CDR3 comprising the amino acid sequence set forth in SEQ ID NO:509; and the heavy chain variable region comprises a CDR1 comprising the amino acid sequence set forth in SEQ ID NO:403, a CDR2 comprising the amino acid sequence set forth in SEQ ID NO:506, and a CDR3 comprising the amino acid sequence set forth in SEQ ID NO:507;
(bx) the light chain variable region comprises a CDR1 comprising the amino acid sequence set forth in SEQ ID NO:312, a CDR2 comprising the amino acid sequence set forth in SEQ ID NO:513, and a CDR3 comprising the amino acid sequence set forth in SEQ ID NO:514; and the heavy chain variable region comprises a CDR1 comprising the amino acid sequence set forth in SEQ ID NO:510, a CDR2 comprising the amino acid sequence set forth in SEQ ID NO:511, and a CDR3 comprising the amino acid sequence set forth in SEQ ID NO:512;
(by) the light chain variable region comprises a CDR1 comprising the amino acid sequence set forth in SEQ ID NO:518, a CDR2 comprising the amino acid sequence set forth in SEQ ID NO:319, and a CDR3 comprising the amino acid sequence set forth in SEQ ID NO:519; and the heavy chain variable region comprises a CDR1 comprising the amino acid sequence set forth in SEQ ID NO:515, a CDR2 comprising the amino acid sequence set forth in SEQ ID NO:516, and a CDR3 comprising the amino acid sequence set forth in SEQ ID NO:517;
(bz) the light chain variable region comprises a CDR1 comprising the amino acid sequence set forth in SEQ ID NO:348, a CDR2 comprising the amino acid sequence set forth in SEQ ID NO:523, and a CDR3 comprising the amino acid sequence set forth in SEQ ID NO:524; and the heavy chain variable region comprises a CDR1 comprising the amino acid sequence set forth in SEQ ID NO:520, a CDR2 comprising the amino acid sequence set forth in SEQ ID NO:521, and a CDR3 comprising the amino acid sequence set forth in SEQ ID NO:522;
(ca) the light chain variable region comprises a CDR1 comprising the amino acid sequence set forth in SEQ ID NO:406, a CDR2 comprising the amino acid sequence set forth in SEQ ID NO:398, and a CDR3 comprising the amino acid sequence set forth in SEQ ID NO:528; and the heavy chain variable region comprises a CDR1 comprising the amino acid sequence set forth in SEQ ID NO:525, a CDR2 comprising the amino acid sequence set forth in SEQ ID NO:526, and a CDR3 comprising the amino acid sequence set forth in SEQ ID NO:527;
(cb) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:530; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:529;
(ce) the light chain variable region comprises a CDR1 comprising the amino acid sequence set forth in SEQ ID NO:428, a CDR2 comprising the amino acid sequence set forth in SEQ ID NO:429, and a CDR3 comprising the amino acid sequence set forth in SEQ ID NO:430; and the heavy chain variable region comprises a CDR1 comprising the amino acid sequence set forth in SEQ ID NO:425, a CDR2 comprising the amino acid sequence set forth in SEQ ID NO:426, and a CDR3 comprising the amino acid sequence set forth in SEQ ID NO:536;
(cf) the light chain variable region comprises a CDR1 comprising the amino acid sequence set forth in SEQ ID NO:540, a CDR2 comprising the amino acid sequence set forth in SEQ ID NO:398, and a CDR3 comprising the amino acid sequence set forth in SEQ ID NO:541; and the heavy chain variable region comprises a CDR1 comprising the amino acid sequence set forth in SEQ ID NO:537, a CDR2 comprising the amino acid sequence set forth in SEQ ID NO:538, and a CDR3 comprising the amino acid sequence set forth in SEQ ID NO:539;
(cg) the light chain variable region comprises a CDR1 comprising the amino acid sequence set forth in SEQ ID NO:406, a CDR2 comprising the amino acid sequence set forth in SEQ ID NO:398, and a CDR3 comprising the amino acid sequence set forth in SEQ ID NO:542; and the heavy chain variable region comprises a CDR1 comprising the amino acid sequence set forth in SEQ ID NO:537, a CDR2 comprising the amino acid sequence set forth in SEQ ID NO:538, and a CDR3 comprising the amino acid sequence set forth in SEQ ID NO:539;
(ci) the light chain variable region comprises a CDR1 comprising the amino acid sequence set forth in SEQ ID NO:547, a CDR2 comprising the amino acid sequence set forth in SEQ ID NO:548, and a CDR3 comprising the amino acid sequence set forth in SEQ ID NO:549; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:546;
(cj) the light chain variable region comprises a CDR1 comprising the amino acid sequence set forth in SEQ ID NO:312, a CDR2 comprising the amino acid sequence set forth in SEQ ID NO:313, and a CDR3 comprising the amino acid sequence set forth in SEQ ID NO:314; and the heavy chain variable region comprises a CDR1 comprising the amino acid sequence set forth in SEQ ID NO:309, a CDR2 comprising the amino acid sequence set forth in SEQ ID NO:310, and a CDR3 comprising the amino acid sequence set forth in SEQ ID NO:550;
(ck) the light chain variable region comprises a CDR1 comprising the amino acid sequence set forth in SEQ ID NO:312, a CDR2 comprising the amino acid sequence set forth in SEQ ID NO:513, and a CDR3 comprising the amino acid sequence set forth in SEQ ID NO:314; and the heavy chain variable region comprises a CDR1 comprising the amino acid sequence set forth in SEQ ID NO:309, a CDR2 comprising the amino acid sequence set forth in SEQ ID NO:310, and a CDR3 comprising the amino acid sequence set forth in SEQ ID NO:550;
(cl) the light chain variable region comprises a CDR1 comprising the amino acid sequence set forth in SEQ ID NO:406, a CDR2 comprising the amino acid sequence set forth in SEQ ID NO:398, and a CDR3 comprising the amino acid sequence set forth in SEQ ID NO:554; and the heavy chain variable region comprises a CDR1 comprising the amino acid sequence set forth in SEQ ID NO:551, a CDR2 comprising the amino acid sequence set forth in SEQ ID NO:552, and a CDR3 comprising the amino acid sequence set forth in SEQ ID NO:553;
(cm) the light chain variable region comprises a CDR1 comprising the amino acid sequence set forth in SEQ ID NO:556, a CDR2 comprising the amino acid sequence set forth in SEQ ID NO:557, and a CDR3 comprising the amino acid sequence set forth in SEQ ID NO:558; and the heavy chain variable region comprises a CDR1 comprising the amino acid sequence set forth in SEQ ID NO:309, a CDR2 comprising the amino acid sequence set forth in SEQ ID NO:310, and a CDR3 comprising the amino acid sequence set forth in SEQ ID NO:555;
(cn) the light chain variable region comprises a CDR1 comprising the amino acid sequence set forth in SEQ ID NO:562, a CDR2 comprising the amino acid sequence set forth in SEQ ID NO:563, and a CDR3 comprising the amino acid sequence set forth in SEQ ID NO:564; and the heavy chain variable region comprises a CDR1 comprising the amino acid sequence set forth in SEQ ID NO:559, a CDR2 comprising the amino acid sequence set forth in SEQ ID NO:560, and a CDR3 comprising the amino acid sequence set forth in SEQ ID NO:561;
(co) the light chain variable region comprises a CDR1 comprising the amino acid sequence set forth in SEQ ID NO:568, a CDR2 comprising the amino acid sequence set forth in SEQ ID NO:504, and a CDR3 comprising the amino acid sequence set forth in SEQ ID NO:569; and the heavy chain variable region comprises a CDR1 comprising the amino acid sequence set forth in SEQ ID NO:565, a CDR2 comprising the amino acid sequence set forth in SEQ ID NO:566, and a CDR3 comprising the amino acid sequence set forth in SEQ ID NO:567;
(cq) the light chain variable region comprises a CDR1 comprising the amino acid sequence set forth in SEQ ID NO:343, a CDR2 comprising the amino acid sequence set forth in SEQ ID NO:575, and a CDR3 comprising the amino acid sequence set forth in SEQ ID NO:576; and the heavy chain variable region comprises a CDR1 comprising the amino acid sequence set forth in SEQ ID NO:403, a CDR2 comprising the amino acid sequence set forth in SEQ ID NO:404, and a CDR3 comprising the amino acid sequence set forth in SEQ ID NO:574;
(cr) the light chain variable region comprises a CDR1 comprising the amino acid sequence set forth in SEQ ID NO:312, a CDR2 comprising the amino acid sequence set forth in SEQ ID NO:313, and a CDR3 comprising the amino acid sequence set forth in SEQ ID NO:314; and the heavy chain variable region comprises a CDR1 comprising the amino acid sequence set forth in SEQ ID NO:411, a CDR2 comprising the amino acid sequence set forth in SEQ ID NO:412, and a CDR3 comprising the amino acid sequence set forth in SEQ ID NO:577;
(cs) the light chain variable region comprises a CDR1 comprising the amino acid sequence set forth in SEQ ID NO:312, a CDR2 comprising the amino acid sequence set forth in SEQ ID NO:313, and a CDR3 comprising the amino acid sequence set forth in SEQ ID NO:314; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:578;
(ct) the light chain variable region comprises a CDR1 comprising the amino acid sequence set forth in SEQ ID NO:580, a CDR2 comprising the amino acid sequence set forth in SEQ ID NO:438, and a CDR3 comprising the amino acid sequence set forth in SEQ ID NO:581; and the heavy chain variable region comprises a CDR1 comprising the amino acid sequence set forth in SEQ ID NO:436, a CDR2 comprising the amino acid sequence set forth in SEQ ID NO:388, and a CDR3 comprising the amino acid sequence set forth in SEQ ID NO:579;
(cu) the light chain variable region comprises a CDR1 comprising the amino acid sequence set forth in SEQ ID NO:585, a CDR2 comprising the amino acid sequence set forth in SEQ ID NO:313, and a CDR3 comprising the amino acid sequence set forth in SEQ ID NO:314; and the heavy chain variable region comprises a CDR1 comprising the amino acid sequence set forth in SEQ ID NO:582, a CDR2 comprising the amino acid sequence set forth in SEQ ID NO:583, and a CDR3 comprising the amino acid sequence set forth in SEQ ID NO:584;
(cv) the light chain variable region comprises a CDR1 comprising the amino acid sequence set forth in SEQ ID NO:312, a CDR2 comprising the amino acid sequence set forth in SEQ ID NO:586, and a CDR3 comprising the amino acid sequence set forth in SEQ ID NO:314; and the heavy chain variable region comprises a CDR1 comprising the amino acid sequence set forth in SEQ ID NO:346, a CDR2 comprising the amino acid sequence set forth in SEQ ID NO:327, and a CDR3 comprising the amino acid sequence set forth in SEQ ID NO:578;
(cw) the light chain variable region comprises a CDR1 comprising the amino acid sequence set forth in SEQ ID NO:588, a CDR2 comprising the amino acid sequence set forth in SEQ ID NO:385, and a CDR3 comprising the amino acid sequence set forth in SEQ ID NO:375; and the heavy chain variable region comprises a CDR1 comprising the amino acid sequence set forth in SEQ ID NO:403, a CDR2 comprising the amino acid sequence set forth in SEQ ID NO:404, and a CDR3 comprising the amino acid sequence set forth in SEQ ID NO:587;
(cx) the light chain variable region comprises a CDR1 comprising the amino acid sequence set forth in SEQ ID NO:318, a CDR2 comprising the amino acid sequence set forth in SEQ ID NO:319, and a CDR3 comprising the amino acid sequence set forth in SEQ ID NO:592; and the heavy chain variable region comprises a CDR1 comprising the amino acid sequence set forth in SEQ ID NO:589, a CDR2 comprising the amino acid sequence set forth in SEQ ID NO:590, and a CDR3 comprising the amino acid sequence set forth in SEQ ID NO:591;
(cy) the light chain variable region comprises a CDR1 comprising the amino acid sequence set forth in SEQ ID NO:926, a CDR2 comprising the amino acid sequence set forth in SEQ ID NO:927, and a CDR3 comprising the amino acid sequence set forth in SEQ ID NO:928; and the heavy chain variable region comprises a CDR1 comprising the amino acid sequence set forth in SEQ ID NO:923, a CDR2 comprising the amino acid sequence set forth in SEQ ID NO:924, and a CDR3 comprising the amino acid sequence set forth in SEQ ID NO:925; or
(cz) the light chain variable region comprises a CDR1 comprising the amino acid sequence set forth in SEQ ID NO:932, a CDR2 comprising the amino acid sequence set forth in SEQ ID NO:933, and a CDR3 comprising the amino acid sequence set forth in SEQ ID NO:934; and the heavy chain variable region comprises a CDR1 comprising the amino acid sequence set forth in SEQ ID NO:929, a CDR2 comprising the amino acid sequence set forth in SEQ ID NO:930, and a CDR3 comprising the amino acid sequence set forth in SEQ ID NO:931.

3. The CAR of claim 1, wherein:
(ab) the light chain variable region comprises an amino acid sequence that has at least about 90% sequence identity to the sequence set forth in SEQ ID NO:7, and the heavy chain variable region comprises an amino acid sequence that has at least about 90% sequence identity to the sequence set forth in SEQ ID NO:8;
(ac) the light chain variable region comprises an amino acid sequence that has at least about 90% sequence identity to the sequence set forth in SEQ ID NO:11, and the heavy chain variable region comprises an amino acid sequence that has at least about 90% sequence identity to the sequence set forth in SEQ ID NO:12;
(ad) the light chain variable region comprises an amino acid sequence that has at least about 90% sequence identity to the sequence set forth in SEQ ID NO:299, and the heavy chain variable region comprises an amino acid sequence that has at least about 90% sequence identity to the sequence set forth in SEQ ID NO:300;
(ae) the light chain variable region comprises an amino acid sequence that has at least about 90% sequence identity to the sequence set forth in SEQ ID NO:15, and the heavy chain variable region comprises an amino acid sequence that has at least about 90% sequence identity to the sequence set forth in SEQ ID NO:16;
(af) the light chain variable region comprises an amino acid sequence that has at least about 90% sequence identity to the sequence set forth in SEQ ID NO:19, and the heavy chain variable region comprises an amino acid sequence that has at least about 90% sequence identity to the sequence set forth in SEQ ID NO:20;
(ag) the light chain variable region comprises an amino acid sequence that has at least about 90% sequence identity to the sequence set forth in SEQ ID NO:23, and the heavy chain variable region comprises an amino acid sequence that has at least about 90% sequence identity to the sequence set forth in SEQ ID NO:24;
(ah) the light chain variable region comprises an amino acid sequence that has at least about 90% sequence identity to the sequence set forth in SEQ ID NO:27, and the heavy chain variable region comprises an amino acid sequence that has at least about 90% sequence identity to the sequence set forth in SEQ ID NO:28;
(ai) the light chain variable region comprises an amino acid sequence that has at least about 90% sequence identity to the sequence set forth in SEQ ID NO:31, and the heavy chain variable region comprises an amino acid sequence that has at least about 90% sequence identity to the sequence set forth in SEQ ID NO:32;
(aj) the light chain variable region comprises an amino acid sequence that has at least about 90% sequence identity to the sequence set forth in SEQ ID NO:35, and the heavy chain variable region comprises an amino acid sequence that has at least about 90% sequence identity to the sequence set forth in SEQ ID NO:36;
(ak) the light chain variable region comprises an amino acid sequence that has at least about 90% sequence identity to the sequence set forth in SEQ ID NO:39, and the heavy chain variable region comprises an amino acid sequence that has at least about 90% sequence identity to the sequence set forth in SEQ ID NO:40;
(al) the light chain variable region comprises an amino acid sequence that has at least about 90% sequence identity to the sequence set forth in SEQ ID NO:43, and the heavy chain variable region comprises an amino acid sequence that has at least about 90% sequence identity to the sequence set forth in SEQ ID NO:44;
(am) the light chain variable region comprises an amino acid sequence that has at least about 90% sequence identity to the sequence set forth in SEQ ID NO:47, and the heavy chain variable region comprises an amino acid sequence that has at least about 90% sequence identity to the sequence set forth in SEQ ID NO:48;
(an) the light chain variable region comprises an amino acid sequence that has at least about 90% sequence identity to the sequence set forth in SEQ ID NO:51, and the heavy chain variable region comprises an amino acid sequence that has at least about 90% sequence identity to the sequence set forth in SEQ ID NO:52;
(ao) the light chain variable region comprises an amino acid sequence that has at least about 90% sequence identity to the sequence set forth in SEQ ID NO:55, and the heavy chain variable region comprises an amino acid sequence that has at least about 90% sequence identity to the sequence set forth in SEQ ID NO:56;
(ap) the light chain variable region comprises an amino acid sequence that has at least about 90% sequence identity to the sequence set forth in SEQ ID NO:59, and the heavy chain variable region comprises an amino acid sequence that has at least about 90% sequence identity to the sequence set forth in SEQ ID NO:60;
(aq) the light chain variable region comprises an amino acid sequence that has at least about 90% sequence identity to the sequence set forth in SEQ ID NO:63, and the heavy chain variable region comprises an amino acid sequence that has at least about 90% sequence identity to the sequence set forth in SEQ ID NO:64;
(ar) the light chain variable region comprises an amino acid sequence that has at least about 90% sequence identity to the sequence set forth in SEQ ID NO:67, and the heavy chain variable region comprises an amino acid sequence that has at least about 90% sequence identity to the sequence set forth in SEQ ID NO:68;
(as) the light chain variable region comprises an amino acid sequence that has at least about 90% sequence identity to the sequence set forth in SEQ ID NO:71, and the heavy chain variable region comprises an amino acid sequence that has at least about 90% sequence identity to the sequence set forth in SEQ ID NO:72;
(at) the light chain variable region comprises an amino acid sequence that has at least about 90% sequence identity to the sequence set forth in SEQ ID NO:75, and the heavy chain variable region comprises an amino acid sequence that has at least about 90% sequence identity to the sequence set forth in SEQ ID NO:76;
(au) the light chain variable region comprises an amino acid sequence that has at least about 90% sequence identity to the sequence set forth in SEQ ID NO:79, and the heavy chain variable region comprises an amino acid sequence that has at least about 90% sequence identity to the sequence set forth in SEQ ID NO:80;
(av) the light chain variable region comprises an amino acid sequence that has at least about 90% sequence identity to the sequence set forth in SEQ ID NO:83, and the heavy chain variable region comprises an amino acid sequence that has at least about 90% sequence identity to the sequence set forth in SEQ ID NO:84;
(aw) the light chain variable region comprises an amino acid sequence that has at least about 90% sequence identity to the sequence set forth in SEQ ID NO:87, and the heavy chain variable region comprises an amino acid sequence that has at least about 90% sequence identity to the sequence set forth in SEQ ID NO:88;
(ax) the light chain variable region comprises an amino acid sequence that has at least about 90% sequence identity to the sequence set forth in SEQ ID NO:91, and the heavy chain variable region comprises an amino acid sequence that has at least about 90% sequence identity to the sequence set forth in SEQ ID NO:92;
(ay) the light chain variable region comprises an amino acid sequence that has at least about 90% sequence identity to the sequence set forth in SEQ ID NO:95, and the heavy chain variable region comprises an amino acid sequence that has at least about 90% sequence identity to the sequence set forth in SEQ ID NO:96;
(az) the light chain variable region comprises an amino acid sequence that has at least about 90% sequence identity to the sequence set forth in SEQ ID NO:99, and the heavy chain variable region comprises an amino acid sequence that has at least about 90% sequence identity to the sequence set forth in SEQ ID NO:100;
(ba) the light chain variable region comprises an amino acid sequence that has at least about 90% sequence identity to the sequence set forth in SEQ ID NO:103, and the heavy chain variable region comprises an amino acid sequence that has at least about 90% sequence identity to the sequence set forth in SEQ ID NO:104;
(bb) the light chain variable region comprises an amino acid sequence that has at least about 90% sequence identity to the sequence set forth in SEQ ID NO:107, and the heavy chain variable region comprises an amino acid sequence that has at least about 90% sequence identity to the sequence set forth in SEQ ID NO:108;
(be) the light chain variable region comprises an amino acid sequence that has at least about 90% sequence identity to the sequence set forth in SEQ ID NO:111, and the heavy chain variable region comprises an amino acid sequence that has at least about 90% sequence identity to the sequence set forth in SEQ ID NO:112;
(be) the light chain variable region comprises an amino acid sequence that has at least about 90% sequence identity to the sequence set forth in SEQ ID NO:119, and the heavy chain variable region comprises an amino acid sequence that has at least about 90% sequence identity to the sequence set forth in SEQ ID NO:120;
(bf) the light chain variable region comprises an amino acid sequence that has at least about 90% sequence identity to the sequence set forth in SEQ ID NO:123, and the heavy chain variable region comprises an amino acid sequence that has at least about 90% sequence identity to the sequence set forth in SEQ ID NO:124;
(bg) the light chain variable region comprises an amino acid sequence that has at least about 90% sequence identity to the sequence set forth in SEQ ID NO:127, and the heavy chain variable region comprises an amino acid sequence that has at least about 90% sequence identity to the sequence set forth in SEQ ID NO:128;
(bh) the light chain variable region comprises an amino acid sequence that has at least about 90% sequence identity to the sequence set forth in SEQ ID NO:131, and the heavy chain variable region comprises an amino acid sequence that has at least about 90% sequence identity to the sequence set forth in SEQ ID NO:132;
(bi) the light chain variable region comprises an amino acid sequence that has at least about 90% sequence identity to the sequence set forth in SEQ ID NO:135, and the heavy chain variable region comprises an amino acid sequence that has at least about 90% sequence identity to the sequence set forth in SEQ ID NO:136;
(bj) the light chain variable region comprises an amino acid sequence that has at least about 90% sequence identity to the sequence set forth in SEQ ID NO:139, and the heavy chain variable region comprises an amino acid sequence that has at least about 90% sequence identity to the sequence set forth in SEQ ID NO:140;
(bl) the light chain variable region comprises an amino acid sequence that has at least about 90% sequence identity to the sequence set forth in SEQ ID NO:147, and the heavy chain variable region comprises an amino acid sequence that has at least about 90% sequence identity to the sequence set forth in SEQ ID NO:148;
(bm) the light chain variable region comprises an amino acid sequence that has at least about 90% sequence identity to the sequence set forth in SEQ ID NO:151, and the heavy chain variable region comprises an amino acid sequence that has at least about 90% sequence identity to the sequence set forth in SEQ ID NO:152;
(bn) the light chain variable region comprises an amino acid sequence that has at least about 90% sequence identity to the sequence set forth in SEQ ID NO:155, and the heavy chain variable region comprises an amino acid sequence that has at least about 90% sequence identity to the sequence set forth in SEQ ID NO:156;
(bo) the light chain variable region comprises an amino acid sequence that has at least about 90% sequence identity to the sequence set forth in SEQ ID NO:159, and the heavy chain variable region comprises an amino acid sequence that has at least about 90% sequence identity to the sequence set forth in SEQ ID NO:160;
(bp) the light chain variable region comprises an amino acid sequence that has at least about 90% sequence identity to the sequence set forth in SEQ ID NO:163, and the heavy chain variable region comprises an amino acid sequence that has at least about 90% sequence identity to the sequence set forth in SEQ ID NO:164;
(bq) the light chain variable region comprises an amino acid sequence that has at least about 90% sequence identity to the sequence set forth in SEQ ID NO:167, and the heavy chain variable region comprises an amino acid sequence that has at least about 90% sequence identity to the sequence set forth in SEQ ID NO:168;
(bs) the light chain variable region comprises an amino acid sequence that has at least about 90% sequence identity to the sequence set forth in SEQ ID NO:175, and the heavy chain variable region comprises an amino acid sequence that has at least about 90% sequence identity to the sequence set forth in SEQ ID NO:176;
(bt) the light chain variable region comprises an amino acid sequence that has at least about 90% sequence identity to the sequence set forth in SEQ ID NO:179, and the heavy chain variable region comprises an amino acid sequence that has at least about 90% sequence identity to the sequence set forth in SEQ ID NO:180;
(bu) the light chain variable region comprises an amino acid sequence that has at least about 90% sequence identity to the sequence set forth in SEQ ID NO:183, and the heavy chain variable region comprises an amino acid sequence that has at least about 90% sequence identity to the sequence set forth in SEQ ID NO:184;
(by) the light chain variable region comprises an amino acid sequence that has at least about 90% sequence identity to the sequence set forth in SEQ ID NO:187, and the heavy chain variable region comprises an amino acid sequence that has at least about 90% sequence identity to the sequence set forth in SEQ ID NO:188;
(bw) the light chain variable region comprises an amino acid sequence that has at least about 90% sequence identity to the sequence set forth in SEQ ID NO:191, and the heavy chain variable region comprises an amino acid sequence that has at least about 90% sequence identity to the sequence set forth in SEQ ID NO:192;
(bx) the light chain variable region comprises an amino acid sequence that has at least about 90% sequence identity to the sequence set forth in SEQ ID NO:195, and the heavy chain variable region comprises an amino acid sequence that has at least about 90% sequence identity to the sequence set forth in SEQ ID NO:196;
(by) the light chain variable region comprises an amino acid sequence that has at least about 90% sequence identity to the sequence set forth in SEQ ID NO:199, and the heavy chain variable region comprises an amino acid sequence that has at least about 90% sequence identity to the sequence set forth in SEQ ID NO:200;
(bz) the light chain variable region comprises an amino acid sequence that has at least about 90% sequence identity to the sequence set forth in SEQ ID NO:203, and the heavy chain variable region comprises an amino acid sequence that has at least about 90% sequence identity to the sequence set forth in SEQ ID NO:204;
(ca) the light chain variable region comprises an amino acid sequence that has at least about 90% sequence identity to the sequence set forth in SEQ ID NO:207, and the heavy chain variable region comprises an amino acid sequence that has at least about 90% sequence identity to the sequence set forth in SEQ ID NO:208;
(cb) the light chain variable region comprises an amino acid sequence that has at least about 90% sequence identity to the sequence set forth in SEQ ID NO:211, and the heavy chain variable region comprises an amino acid sequence that has at least about 90% sequence identity to the sequence set forth in SEQ ID NO:212;
(ce) the light chain variable region comprises an amino acid sequence that has at least about 90% sequence identity to the sequence set forth in SEQ ID NO:223, and the heavy chain variable region comprises an amino acid sequence that has at least about 90% sequence identity to the sequence set forth in SEQ ID NO:224;
(cf) the light chain variable region comprises an amino acid sequence that has at least about 90% sequence identity to the sequence set forth in SEQ ID NO:227, and the heavy chain variable region comprises an amino acid sequence that has at least about 90% sequence identity to the sequence set forth in SEQ ID NO:228;
(cg) the light chain variable region comprises an amino acid sequence that has at least about 90% sequence identity to the sequence set forth in SEQ ID NO:231, and the heavy chain variable region comprises an amino acid sequence that has at least about 90% sequence identity to the sequence set forth in SEQ ID NO:232;
(ci) the light chain variable region comprises an amino acid sequence that has at least about 90% sequence identity to the sequence set forth in SEQ ID NO:239, and the heavy chain variable region comprises an amino acid sequence that has at least about 90% sequence identity to the sequence set forth in SEQ ID NO:240;
(cj) the light chain variable region comprises an amino acid sequence that has at least about 90% sequence identity to the sequence set forth in SEQ ID NO:243, and the heavy chain variable region comprises an amino acid sequence that has at least about 90% sequence identity to the sequence set forth in SEQ ID NO:244;
(ck) the light chain variable region comprises an amino acid sequence that has at least about 90% sequence identity to the sequence set forth in SEQ ID NO:247, and the heavy chain variable region comprises an amino acid sequence that has at least about 90% sequence identity to the sequence set forth in SEQ ID NO:248;
(cl) the light chain variable region comprises an amino acid sequence that has at least about 90% sequence identity to the sequence set forth in SEQ ID NO:251, and the heavy chain variable region comprises an amino acid sequence that has at least about 90% sequence identity to the sequence set forth in SEQ ID NO:252;
(cm) the light chain variable region comprises an amino acid sequence that has at least about 90% sequence identity to the sequence set forth in SEQ ID NO:255, and the heavy chain variable region comprises an amino acid sequence that has at least about 90% sequence identity to the sequence set forth in SEQ ID NO:256;
(cn) the light chain variable region comprises an amino acid sequence that has at least about 90% sequence identity to the sequence set forth in SEQ ID NO:259, and the heavy chain variable region comprises an amino acid sequence that has at least about 90% sequence identity to the sequence set forth in SEQ ID NO:260;
(co) the light chain variable region comprises an amino acid sequence that has at least about 90% sequence identity to the sequence set forth in SEQ ID NO:263, and the heavy chain variable region comprises an amino acid sequence that has at least about 90% sequence identity to the sequence set forth in SEQ ID NO:264;
(cq) the light chain variable region comprises an amino acid sequence that has at least about 90% sequence identity to the sequence set forth in SEQ ID NO:271, and the heavy chain variable region comprises an amino acid sequence that has at least about 90% sequence identity to the sequence set forth in SEQ ID NO:272;
(cr) the light chain variable region comprises an amino acid sequence that has at least about 90% sequence identity to the sequence set forth in SEQ ID NO:275, and the heavy chain variable region comprises an amino acid sequence that has at least about 90% sequence identity to the sequence set forth in SEQ ID NO:276;
(cs) the light chain variable region comprises an amino acid sequence that has at least about 90% sequence identity to the sequence set forth in SEQ ID NO:279, and the heavy chain variable region comprises an amino acid sequence that has at least about 90% sequence identity to the sequence set forth in SEQ ID NO:280;
(ct) the light chain variable region comprises an amino acid sequence that has at least about 90% sequence identity to the sequence set forth in SEQ ID NO:283, and the heavy chain variable region comprises an amino acid sequence that has at least about 90% sequence identity to the sequence set forth in SEQ ID NO:284;
(cu) the light chain variable region comprises an amino acid sequence that has at least about 90% sequence identity to the sequence set forth in SEQ ID NO:287, and the heavy chain variable region comprises an amino acid sequence that has at least about 90% sequence identity to the sequence set forth in SEQ ID NO:288;
(cv) the light chain variable region comprises an amino acid sequence that has at least about 90% sequence identity to the sequence set forth in SEQ ID NO:291, and the heavy chain variable region comprises an amino acid sequence that has at least about 90% sequence identity to the sequence set forth in SEQ ID NO:292;
(cw) the light chain variable region comprises an amino acid sequence that has at least about 90% sequence identity to the sequence set forth in SEQ ID NO:303, and the heavy chain variable region comprises an amino acid sequence that has at least about 90% sequence identity to the sequence set forth in SEQ ID NO:304;
(cx) the light chain variable region comprises an amino acid sequence that has at least about 90% sequence identity to the sequence set forth in SEQ ID NO:295, and the heavy chain variable region comprises an amino acid sequence that has at least about 90% sequence identity to the sequence set forth in SEQ ID NO:296;
(cy) the light chain variable region comprises an amino acid sequence that has at least about 90% sequence identity to the sequence set forth in SEQ ID NO:915, and the heavy chain variable region comprises an amino acid sequence that has at least about 90% sequence identity to the sequence set forth in SEQ ID NO:917; or
(cz) the light chain variable region comprises an amino acid sequence that has at least about 90% sequence identity to the sequence set forth in SEQ ID NO:919, and the heavy chain variable region comprises an amino acid sequence that has at least about 90% sequence identity to the sequence set forth in SEQ ID NO:921.

4. The CAR of claim 3, wherein:
(ab) the light chain variable region comprises the amino acid sequence set forth in SEQ ID NO:7, and the heavy chain variable region comprises the amino acid sequence set forth in SEQ ID NO:8;
(ac) the light chain variable region comprises the amino acid sequence set forth in SEQ ID NO:11, and the heavy chain variable region comprises the amino acid sequence set forth in SEQ ID NO:12;
(ad) the light chain variable region comprises the amino acid sequence set forth in SEQ ID NO:299, and the heavy chain variable region comprises the amino acid sequence set forth in SEQ ID NO:300;
(ae) the light chain variable region comprises the amino acid sequence set forth in SEQ ID NO:15, and the heavy chain variable region comprises the amino acid sequence set forth in SEQ ID NO:16;
(af) the light chain variable region comprises the amino acid sequence set forth in SEQ ID NO:19, and the heavy chain variable region comprises the amino acid sequence set forth in SEQ ID NO:20;
(ag) the light chain variable region comprises the amino acid sequence set forth in SEQ ID NO:23, and the heavy chain variable region comprises the amino acid sequence set forth in SEQ ID NO:24;
(ah) the light chain variable region comprises the amino acid sequence set forth in SEQ ID NO:27, and the heavy chain variable region comprises the amino acid sequence set forth in SEQ ID NO:28;
(ai) the light chain variable region comprises the amino acid sequence set forth in SEQ ID NO:31, and the heavy chain variable region comprises the amino acid sequence set forth in SEQ ID NO:32;
(aj) the light chain variable region comprises the amino acid sequence set forth in SEQ ID NO:35, and the heavy chain variable region comprises the amino acid sequence set forth in SEQ ID NO:36;
(ak) the light chain variable region comprises the amino acid sequence set forth in SEQ ID NO:39, and the heavy chain variable region comprises the amino acid sequence set forth in SEQ ID NO:40;
(al) the light chain variable region comprises the amino acid sequence set forth in SEQ ID NO:43, and the heavy chain variable region comprises the amino acid sequence set forth in SEQ ID NO:44;
(am) the light chain variable region comprises the amino acid sequence set forth in SEQ ID NO:47, and the heavy chain variable region comprises the amino acid sequence set forth in SEQ ID NO:48;
(an) the light chain variable region comprises the amino acid sequence set forth in SEQ ID NO:51, and the heavy chain variable region comprises the amino acid sequence set forth in SEQ ID NO:52;
(ao) the light chain variable region comprises the amino acid sequence set forth in SEQ ID NO:55, and the heavy chain variable region comprises the amino acid sequence set forth in SEQ ID NO:56;
(ap) the light chain variable region comprises the amino acid sequence set forth in SEQ ID NO:59, and the heavy chain variable region comprises the amino acid sequence set forth in SEQ ID NO:60;
(aq) the light chain variable region comprises the amino acid sequence set forth in SEQ ID NO:63, and the heavy chain variable region comprises the amino acid sequence set forth in SEQ ID NO:64;
(ar) the light chain variable region comprises the amino acid sequence set forth in SEQ ID NO:67, and the heavy chain variable region comprises the amino acid sequence set forth in SEQ ID NO:68;
(as) the light chain variable region comprises the amino acid sequence set forth in SEQ ID NO:71, and the heavy chain variable region comprises the amino acid sequence set forth in SEQ ID NO:72;
(at) the light chain variable region comprises the amino acid sequence set forth in SEQ ID NO:75, and the heavy chain variable region comprises the amino acid sequence set forth in SEQ ID NO:76;
(au) the light chain variable region comprises the amino acid sequence set forth in SEQ ID NO:79, and the heavy chain variable region comprises the amino acid sequence set forth in SEQ ID NO:80;
(av) the light chain variable region comprises the amino acid sequence set forth in SEQ ID NO:83, and the heavy chain variable region comprises the amino acid sequence set forth in SEQ ID NO:84;
(aw) the light chain variable region comprises the amino acid sequence set forth in SEQ ID NO:87, and the heavy chain variable region comprises the amino acid sequence set forth in SEQ ID NO:88;
(ax) the light chain variable region comprises the amino acid sequence set forth in SEQ ID NO:91, and the heavy chain variable region comprises the amino acid sequence set forth in SEQ ID NO:92;
(ay) the light chain variable region comprises the amino acid sequence set forth in SEQ ID NO:95, and the heavy chain variable region comprises the amino acid sequence set forth in SEQ ID NO:96;
(az) the light chain variable region comprises the amino acid sequence set forth in SEQ ID NO:99, and the heavy chain variable region comprises the amino acid sequence set forth in SEQ ID NO:100;
(ba) the light chain variable region comprises the amino acid sequence set forth in SEQ ID NO:103, and the heavy chain variable region comprises the amino acid sequence set forth in SEQ ID NO:104;
(bb) the light chain variable region comprises the amino acid sequence set forth in SEQ ID NO:107, and the heavy chain variable region comprises the amino acid sequence set forth in SEQ ID NO:108;
(bc) the light chain variable region comprises the amino acid sequence set forth in SEQ ID NO:111, and the heavy chain variable region comprises the amino acid sequence set forth in SEQ ID NO:112;
(be) the light chain variable region comprises the amino acid sequence set forth in SEQ ID NO:119, and the heavy chain variable region comprises the amino acid sequence set forth in SEQ ID NO:120;
(bf) the light chain variable region comprises the amino acid sequence set forth in SEQ ID NO:123, and the heavy chain variable region comprises the amino acid sequence set forth in SEQ ID NO:124;
(bg) the light chain variable region comprises the amino acid sequence set forth in SEQ ID NO:127, and the heavy chain variable region comprises the amino acid sequence set forth in SEQ ID NO:128;
(bh) the light chain variable region comprises the amino acid sequence set forth in SEQ ID NO:131, and the heavy chain variable region comprises the amino acid sequence set forth in SEQ ID NO:132;
(bi) the light chain variable region comprises the amino acid sequence set forth in SEQ ID NO:135, and the heavy chain variable region comprises the amino acid sequence set forth in SEQ ID NO:136;
(bj) the light chain variable region comprises the amino acid sequence set forth in SEQ ID NO:139, and the heavy chain variable region comprises the amino acid sequence set forth in SEQ ID NO:140;
(bl) the light chain variable region comprises the amino acid sequence set forth in SEQ ID NO:147, and the heavy chain variable region comprises the amino acid sequence set forth in SEQ ID NO:148;
(bm) the light chain variable region comprises the amino acid sequence set forth in SEQ ID NO:151, and the heavy chain variable region comprises the amino acid sequence set forth in SEQ ID NO:152;
(bn) the light chain variable region comprises the amino acid sequence set forth in SEQ ID NO:155, and the heavy chain variable region comprises the amino acid sequence set forth in SEQ ID NO:156;
(bo) the light chain variable region comprises the amino acid sequence set forth in SEQ ID NO:159, and the heavy chain variable region comprises the amino acid sequence set forth in SEQ ID NO:160;
(bp) the light chain variable region comprises the amino acid sequence set forth in SEQ ID NO:163, and the heavy chain variable region comprises the amino acid sequence set forth in SEQ ID NO:164;
(bq) the light chain variable region comprises the amino acid sequence set forth in SEQ ID NO:167, and the heavy chain variable region comprises the amino acid sequence set forth in SEQ ID NO:168;
(bs) the light chain variable region comprises the amino acid sequence set forth in SEQ ID NO:175, and the heavy chain variable region comprises the amino acid sequence set forth in SEQ ID NO:176;
(bt) the light chain variable region comprises the amino acid sequence set forth in SEQ ID NO:179, and the heavy chain variable region comprises the amino acid sequence set forth in SEQ ID NO:180;
(bu) the light chain variable region comprises the amino acid sequence set forth in SEQ ID NO:183, and the heavy chain variable region comprises the amino acid sequence set forth in SEQ ID NO:184;
(by) the light chain variable region comprises the amino acid sequence set forth in SEQ ID NO:187, and the heavy chain variable region comprises the amino acid sequence set forth in SEQ ID NO:188;
(bw) the light chain variable region comprises the amino acid sequence set forth in SEQ ID NO:191, and the heavy chain variable region comprises the amino acid sequence set forth in SEQ ID NO:192;
(bx) the light chain variable region comprises the amino acid sequence set forth in SEQ ID NO:195, and the heavy chain variable region comprises the amino acid sequence set forth in SEQ ID NO:196;
(by) the light chain variable region comprises the amino acid sequence set forth in SEQ ID NO:199, and the heavy chain variable region comprises the amino acid sequence set forth in SEQ ID NO:200;
(bz) the light chain variable region comprises the amino acid sequence set forth in SEQ ID NO:203, and the heavy chain variable region comprises the amino acid sequence set forth in SEQ ID NO:204;
(ca) the light chain variable region comprises the amino acid sequence set forth in SEQ ID NO:207, and the heavy chain variable region comprises the amino acid sequence set forth in SEQ ID NO:208;
(cb) the light chain variable region comprises the amino acid sequence set forth in SEQ ID NO:211, and the heavy chain variable region comprises the amino acid sequence set forth in SEQ ID NO:212;
(ce) the light chain variable region comprises the amino acid sequence set forth in SEQ ID NO:223, and the heavy chain variable region comprises the amino acid sequence set forth in SEQ ID NO:224;
(cf) the light chain variable region comprises the amino acid sequence set forth in SEQ ID NO:227, and the heavy chain variable region comprises the amino acid sequence set forth in SEQ ID NO:228;
(cg) the light chain variable region comprises the amino acid sequence set forth in SEQ ID NO:231, and the heavy chain variable region comprises the amino acid sequence set forth in SEQ ID NO:232;
(ci) the light chain variable region comprises the amino acid sequence set forth in SEQ ID NO:239, and the heavy chain variable region comprises the amino acid sequence set forth in SEQ ID NO:240;
(cj) the light chain variable region comprises the amino acid sequence set forth in SEQ ID NO:243, and the heavy chain variable region comprises the amino acid sequence set forth in SEQ ID NO:244;
(ck) the light chain variable region comprises the amino acid sequence set forth in SEQ ID NO:247, and the heavy chain variable region comprises the amino acid sequence set forth in SEQ ID NO:248;
(cl) the light chain variable region comprises the amino acid sequence set forth in SEQ ID NO:251, and the heavy chain variable region comprises the amino acid sequence set forth in SEQ ID NO:252;
(cm) the light chain variable region comprises the amino acid sequence set forth in SEQ ID NO:255, and the heavy chain variable region comprises the amino acid sequence set forth in SEQ ID NO:256;
(cn) the light chain variable region comprises the amino acid sequence set forth in SEQ ID NO:259, and the heavy chain variable region comprises the amino acid sequence set forth in SEQ ID NO:260;
(co) the light chain variable region comprises the amino acid sequence set forth in SEQ ID NO:263, and the heavy chain variable region comprises the amino acid sequence set forth in SEQ ID NO:264;
(cq) the light chain variable region comprises the amino acid sequence set forth in SEQ ID NO:271, and the heavy chain variable region comprises the amino acid sequence set forth in SEQ ID NO:272;
(cr) the light chain variable region comprises the amino acid sequence set forth in SEQ ID NO:275, and the heavy chain variable region comprises the amino acid sequence set forth in SEQ ID NO:276;
(cs) the light chain variable region comprises the amino acid sequence set forth in SEQ ID NO:279, and the heavy chain variable region comprises the amino acid sequence set forth in SEQ ID NO:280;
(ct) the light chain variable region comprises the amino acid sequence set forth in SEQ ID NO:283, and the heavy chain variable region comprises the amino acid sequence set forth in SEQ ID NO:284;
(cu) the light chain variable region comprises the amino acid sequence set forth in SEQ ID NO:287, and the heavy chain variable region comprises the amino acid sequence set forth in SEQ ID NO:288;
(cv) the light chain variable region comprises the amino acid sequence set forth in SEQ ID NO:291, and the heavy chain variable region comprises the amino acid sequence set forth in SEQ ID NO:292;
(cw) the light chain variable region comprises the amino acid sequence set forth in SEQ ID NO:303, and the heavy chain variable region comprises the amino acid sequence set forth in SEQ ID NO:304;
(cx) the light chain variable region comprises the amino acid sequence set forth in SEQ ID NO:295, and the heavy chain variable region comprises the amino acid sequence set forth in SEQ ID NO:296;
(cy) the light chain variable region comprises the amino acid sequence set forth in SEQ ID NO:917, and the heavy chain variable region comprises the amino acid sequence set forth in SEQ ID NO:915; or
(cz) the light chain variable region comprises the amino acid sequence set forth in SEQ ID NO:921, and the heavy chain variable region comprises the amino acid sequence set forth in SEQ ID NO:919.

5. The CAR of claim 1, wherein the extracellular antigen-binding domain comprises a single-chain variable fragment (scFv), or a human scFv.

6. The CAR of claim 1, wherein the: a) transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide, or a combination thereof; b) the intracellular domain comprises a CD3ζ polypeptide; and/or c) the intracellular domain further comprises at least one signaling region.

7. The CAR of claim 6, wherein the at least one signaling region comprises a CD28 polypeptide, a 4-1BB polypeptide, an OX40 polypeptide, an ICOS polypeptide, a PD-1 polypeptide, a CTLA-4 polypeptide, a LAG-3 polypeptide, a 2B4 polypeptide, a BTLA polypeptide, a synthetic peptide, or a combination thereof, and/or the at least one signaling region comprises at least one co-stimulatory signaling region.

8. The CAR of claim 7, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

9. The CAR of claim 1, wherein:
(a) the transmembrane domain comprises a CD28 polypeptide, and the intracellular domain comprises a CD3ζ polypeptide and a co-stimulatory signaling region that comprises a CD28 polypeptide;
(b) the transmembrane domain comprises a CD8 polypeptide, and the intracellular domain comprises a CD3ζ polypeptide and a co-stimulatory signaling region that comprises a 4-1BB polypeptide; or
(c) the transmembrane domain comprises a CD28 polypeptide, and the intracellular domain comprises a CD3ζ polypeptide and a co-stimulatory signaling region that comprises a 4-1BB polypeptide.

10. An immunoresponsive cell comprising the CAR of claim 1.

11. The immunoresponsive cell of claim 10, wherein the immunoresponsive cell is further transduced with a nucleic acid molecule encoding at least one co-stimulatory ligand such that the immunoresponsive cell expresses the at least one co-stimulatory ligand; or a nucleic acid molecule encoding at least one cytokine such that the immunoresponsive cell secretes the at least one cytokine.

12. The immunoresponsive cell of claim 10, wherein the immunoresponsive cell is selected from the group consisting of a T cell, a Natural Killer (NK) cell, a cytotoxic T lymphocyte (CTL), a regulatory T cell, a human embryonic stem cell, a lymphoid progenitor cell, a T cell-precursor cell, and a pluripotent stem cell from which lymphoid cells may be differentiated.

13. The immunoresponsive cell of claim 12, wherein the immunoresponsive cell is a T cell.

14. A nucleic acid molecule encoding the CAR of claim 1.

15. A vector comprising the nucleic acid molecule of claim 14.

16. A host cell expressing the nucleic acid molecule of claim 14.

17. A method for producing an immunoresponsive cell that binds to Fc Receptor-Like 5 (FcRL5), comprising introducing into an immunoresponsive cell a nucleic acid molecule that encodes the CAR of claim 1.

18. A pharmaceutical composition comprising an effective amount of the immunoresponsive cell of claim 10 and a pharmaceutically acceptable excipient.

19. A kit for treating a tumor, the kit comprising the immunoresponsive cell of claim 10 and written instructions for using the immunoresponsive cell for treating a subject having a tumor.

20. A method of reducing tumor burden in a subject, or increasing or lengthening survival of a subject having tumor, the method comprising administering an effective amount of an immunoresponsive cell comprising a chimeric antigen receptor (CAR) to the subject, wherein the CAR comprises an extracellular antigen-binding domain, a transmembrane domain, and an intracellular domain, wherein the extracellular antigen-binding domain specifically binds to Fc Receptor-Like 5 (FcRL5) and comprises:
(aa) a light chain variable region comprising a CDR1, a CDR2, and a CDR3 of the light chain variable region sequence set forth in SEQ ID NO:3, and a heavy chain variable region comprising a CDR1, a CDR2, and a CDR3 of the heavy chain variable region sequence set forth in SEQ ID NO:4;
(ab) a light chain variable region comprising a CDR1, a CDR2, and a CDR3 of the light chain variable region sequence set forth in SEQ ID NO:7, and a heavy chain variable region comprising a CDR1, a CDR2, and a CDR3 of the heavy chain variable region sequence set forth in SEQ ID NO:8;
(ac) a light chain variable region comprising a CDR1, a CDR2, and a CDR3 of the light chain variable region sequence set forth in SEQ ID NO:11, and a heavy chain variable region comprising a CDR1, a CDR2, and a CDR3 of the heavy chain variable region sequence set forth in SEQ ID NO:12;
(ad) a light chain variable region comprising a CDR1, a CDR2, and a CDR3 of the light chain variable region sequence set forth in SEQ ID NO:299, and a heavy chain variable region comprising a CDR1, a CDR2, and a CDR3 of the heavy chain variable region sequence set forth in SEQ ID NO:300;
(ae) a light chain variable region comprising a CDR1, a CDR2, and a CDR3 of the light chain variable region sequence set forth in SEQ ID NO:15, and a heavy chain variable region comprising a CDR1, a CDR2, and a CDR3 of the heavy chain variable region sequence set forth in SEQ ID NO:16;
(af) a light chain variable region comprising a CDR1, a CDR2, and a CDR3 of the light chain variable region sequence set forth in SEQ ID NO:19, and a heavy chain variable region comprising a CDR1, a CDR2, and a CDR3 of the heavy chain variable region sequence set forth in SEQ ID NO:20;
(ag) a light chain variable region comprising a CDR1, a CDR2, and a CDR3 of the light chain variable region sequence set forth in SEQ ID NO:23, and a heavy chain variable region comprising a CDR1, a CDR2, and a CDR3 of the heavy chain variable region sequence set forth in SEQ ID NO:24;
(ah) a light chain variable region comprising a CDR1, a CDR2, and a CDR3 of the light chain variable region sequence set forth in SEQ ID NO:27, and a heavy chain variable region comprising a CDR1, a CDR2, and a CDR3 of the heavy chain variable region sequence set forth in SEQ ID NO:28;
(ai) a light chain variable region comprising a CDR1, a CDR2, and a CDR3 of the light chain variable region sequence set forth in SEQ ID NO:31, and a heavy chain variable region comprising a CDR1, a CDR2, and a CDR3 of the heavy chain variable region sequence set forth in SEQ ID NO:32;
(aj) a light chain variable region comprising a CDR1, a CDR2, and a CDR3 of the light chain variable region sequence set forth in SEQ ID NO:35, and a heavy chain variable region comprising a CDR1, a CDR2, and a CDR3 of the heavy chain variable region sequence set forth in SEQ ID NO:36;
(ak) a light chain variable region comprising a CDR1, a CDR2, and a CDR3 of the light chain variable region sequence set forth in SEQ ID NO:39, and a heavy chain variable region comprising a CDR1, a CDR2, and a CDR3 of the heavy chain variable region sequence set forth in SEQ ID NO:40;
(al) a light chain variable region comprising a CDR1, a CDR2, and a CDR3 of the light chain variable region sequence set forth in SEQ ID NO:43, and a heavy chain variable region comprising a CDR1, a CDR2, and a CDR3 of the heavy chain variable region sequence set forth in SEQ ID NO:44;
(am) a light chain variable region comprising a CDR1, a CDR2, and a CDR3 of the light chain variable region sequence set forth in SEQ ID NO:47, and a heavy chain variable region comprising a CDR1, a CDR2, and a CDR3 of the heavy chain variable region sequence set forth in SEQ ID NO:48;
(an) a light chain variable region comprising a CDR1, a CDR2, and a CDR3 of the light chain variable region sequence set forth in SEQ ID NO:51, and a heavy chain variable region comprising a CDR1, a CDR2, and a CDR3 of the heavy chain variable region sequence set forth in SEQ ID NO:52;
(ao) a light chain variable region comprising a CDR1, a CDR2, and a CDR3 of the light chain variable region sequence set forth in SEQ ID NO:55, and a heavy chain variable region comprising a CDR1, a CDR2, and a CDR3 of the heavy chain variable region sequence set forth in SEQ ID NO:56;
(ap) a light chain variable region comprising a CDR1, a CDR2, and a CDR3 of the light chain variable region sequence set forth in SEQ ID NO:59, and a heavy chain variable region comprising a CDR1, a CDR2, and a CDR3 of the heavy chain variable region sequence set forth in SEQ ID NO:60;
(aq) a light chain variable region comprising a CDR1, a CDR2, and a CDR3 of the light chain variable region sequence set forth in SEQ ID NO:63, and a heavy chain variable region comprising a CDR1, a CDR2, and a CDR3 of the heavy chain variable region sequence set forth in SEQ ID NO:64;
(ar) a light chain variable region comprising a CDR1, a CDR2, and a CDR3 of the light chain variable region sequence set forth in SEQ ID NO:67, and a heavy chain variable region comprising a CDR1, a CDR2, and a CDR3 of the heavy chain variable region sequence set forth in SEQ ID NO:68;
(as) a light chain variable region comprising a CDR1, a CDR2, and a CDR3 of the light chain variable region sequence set forth in SEQ ID NO:71, and a heavy chain variable region comprising a CDR1, a CDR2, and a CDR3 of the heavy chain variable region sequence set forth in SEQ ID NO:72;
(at) a light chain variable region comprising a CDR1, a CDR2, and a CDR3 of the light chain variable region sequence set forth in SEQ ID NO:75, and a heavy chain variable region comprising a CDR1, a CDR2, and a CDR3 of the heavy chain variable region sequence set forth in SEQ ID NO:76;
(au) a light chain variable region comprising a CDR1, a CDR2, and a CDR3 of the light chain variable region sequence set forth in SEQ ID NO:79, and a heavy chain variable region comprising a CDR1, a CDR2, and a CDR3 of the heavy chain variable region sequence set forth in SEQ ID NO:80;
(av) a light chain variable region comprising a CDR1, a CDR2, and a CDR3 of the light chain variable region sequence set forth in SEQ ID NO:83, and a heavy chain variable region comprising a CDR1, a CDR2, and a CDR3 of the heavy chain variable region sequence set forth in SEQ ID NO:84;
(aw) a light chain variable region comprising a CDR1, a CDR2, and a CDR3 of the light chain variable region sequence set forth in SEQ ID NO:87, and a heavy chain variable region comprising a CDR1, a CDR2, and a CDR3 of the heavy chain variable region sequence set forth in SEQ ID NO:88;
(ax) a light chain variable region comprising a CDR1, a CDR2, and a CDR3 of the light chain variable region sequence set forth in SEQ ID NO:91, and a heavy chain variable region comprising a CDR1, a CDR2, and a CDR3 of the heavy chain variable region sequence set forth in SEQ ID NO:92;
(ay) a light chain variable region comprising a CDR1, a CDR2, and a CDR3 of the light chain variable region sequence set forth in SEQ ID NO:95, and a heavy chain variable region comprising a CDR1, a CDR2, and a CDR3 of the heavy chain variable region sequence set forth in SEQ ID NO:96;
(az) a light chain variable region comprising a CDR1, a CDR2, and a CDR3 of the light chain variable region sequence set forth in SEQ ID NO:99, and a heavy chain variable region comprising a CDR1, a CDR2, and a CDR3 of the heavy chain variable region sequence set forth in SEQ ID NO:100;
(ba) a light chain variable region comprising a CDR1, a CDR2, and a CDR3 of the light chain variable region sequence set forth in SEQ ID NO:103, and a heavy chain variable region comprising a CDR1, a CDR2, and a CDR3 of the heavy chain variable region sequence set forth in SEQ ID NO:104;
(bb) a light chain variable region comprising a CDR1, a CDR2, and a CDR3 of the light chain variable region sequence set forth in SEQ ID NO:107, and a heavy chain variable region comprising a CDR1, a CDR2, and a CDR3 of the heavy chain variable region sequence set forth in SEQ ID NO:108;
(bc) a light chain variable region comprising a CDR1, a CDR2, and a CDR3 of the light chain variable region sequence set forth in SEQ ID NO:111, and a heavy chain variable region comprising a CDR1, a CDR2, and a CDR3 of the heavy chain variable region sequence set forth in SEQ ID NO:112;
(bd) a light chain variable region comprising a CDR1, a CDR2, and a CDR3 of the light chain variable region sequence set forth in SEQ ID NO:115, and a heavy chain variable region comprising a CDR1, a CDR2, and a CDR3 of the heavy chain variable region sequence set forth in SEQ ID NO:116;
(be) a light chain variable region comprising a CDR1, a CDR2, and a CDR3 of the light chain variable region sequence set forth in SEQ ID NO:119, and a heavy chain variable region comprising a CDR1, a CDR2, and a CDR3 of the heavy chain variable region sequence set forth in SEQ ID NO:120;
(bf) a light chain variable region comprising a CDR1, a CDR2, and a CDR3 of the light chain variable region sequence set forth in SEQ ID NO:123, and a heavy chain variable region comprising a CDR1, a CDR2, and a CDR3 of the heavy chain variable region sequence set forth in SEQ ID NO:124;
(bg) a light chain variable region comprising a CDR1, a CDR2, and a CDR3 of the light chain variable region sequence set forth in SEQ ID NO:127, and a heavy chain variable region comprising a CDR1, a CDR2, and a CDR3 of the heavy chain variable region sequence set forth in SEQ ID NO:128;
(bh) a light chain variable region comprising a CDR1, a CDR2, and a CDR3 of the light chain variable region sequence set forth in SEQ ID NO:131, and a heavy chain variable region comprising a CDR1, a CDR2, and a CDR3 of the heavy chain variable region sequence set forth in SEQ ID NO:132;
(bi) a light chain variable region comprising a CDR1, a CDR2, and a CDR3 of the light chain variable region sequence set forth in SEQ ID NO:135, and a heavy chain variable region comprising a CDR1, a CDR2, and a CDR3 of the heavy chain variable region sequence set forth in SEQ ID NO:136;
(bj) a light chain variable region comprising a CDR1, a CDR2, and a CDR3 of the light chain variable region sequence set forth in SEQ ID NO:139, and a heavy chain variable region comprising a CDR1, a CDR2, and a CDR3 of the heavy chain variable region sequence set forth in SEQ ID NO:140;
(bk) a light chain variable region comprising a CDR1, a CDR2, and a CDR3 of the light chain variable region sequence set forth in SEQ ID NO:143, and a heavy chain variable region comprising a CDR1, a CDR2, and a CDR3 of the heavy chain variable region sequence set forth in SEQ ID NO:144;
(bl) a light chain variable region comprising a CDR1, a CDR2, and a CDR3 of the light chain variable region sequence set forth in SEQ ID NO:147, and a heavy chain variable region comprising a CDR1, a CDR2, and a CDR3 of the heavy chain variable region sequence set forth in SEQ ID NO:148;
(bm) a light chain variable region comprising a CDR1, a CDR2, and a CDR3 of the light chain variable region sequence set forth in SEQ ID NO:151, and a heavy chain variable region comprising a CDR1, a CDR2, and a CDR3 of the heavy chain variable region sequence set forth in SEQ ID NO:152;
(bn) a light chain variable region comprising a CDR1, a CDR2, and a CDR3 of the light chain variable region sequence set forth in SEQ ID NO:155, and a heavy chain variable region comprising a CDR1, a CDR2, and a CDR3 of the heavy chain variable region sequence set forth in SEQ ID NO:156;
(bo) a light chain variable region comprising a CDR1, a CDR2, and a CDR3 of the light chain variable region sequence set forth in SEQ ID NO:159, and a heavy chain variable region comprising a CDR1, a CDR2, and a CDR3 of the heavy chain variable region sequence set forth in SEQ ID NO:160;
(bp) a light chain variable region comprising a CDR1, a CDR2, and a CDR3 of the light chain variable region sequence set forth in SEQ ID NO:163, and a heavy chain variable region comprising a CDR1, a CDR2, and a CDR3 of the heavy chain variable region sequence set forth in SEQ ID NO:164;
(bq) a light chain variable region comprising a CDR1, a CDR2, and a CDR3 of the light chain variable region sequence set forth in SEQ ID NO:167, and a heavy chain variable region comprising a CDR1, a CDR2, and a CDR3 of the heavy chain variable region sequence set forth in SEQ ID NO:168;
(br) a light chain variable region comprising a CDR1, a CDR2, and a CDR3 of the light chain variable region sequence set forth in SEQ ID NO:171, and a heavy chain variable region comprising a CDR1, a CDR2, and a CDR3 of the heavy chain variable region sequence set forth in SEQ ID NO:172;
(bs) a light chain variable region comprising a CDR1, a CDR2, and a CDR3 of the light chain variable region sequence set forth in SEQ ID NO:175, and a heavy chain variable region comprising a CDR1, a CDR2, and a CDR3 of the heavy chain variable region sequence set forth in SEQ ID NO:176;
(bt) a light chain variable region comprising a CDR1, a CDR2, and a CDR3 of the light chain variable region sequence set forth in SEQ ID NO:179, and a heavy chain variable region comprising a CDR1, a CDR2, and a CDR3 of the heavy chain variable region sequence set forth in SEQ ID NO:180;
(bu) a light chain variable region comprising a CDR1, a CDR2, and a CDR3 of the light chain variable region sequence set forth in SEQ ID NO:183, and a heavy chain variable region comprising a CDR1, a CDR2, and a CDR3 of the heavy chain variable region sequence set forth in SEQ ID NO:184;
(by) a light chain variable region comprising a CDR1, a CDR2, and a CDR3 of the light chain variable region sequence set forth in SEQ ID NO:187, and a heavy chain variable region comprising a CDR1, a CDR2, and a CDR3 of the heavy chain variable region sequence set forth in SEQ ID NO:188;
(bw) a light chain variable region comprising a CDR1, a CDR2, and a CDR3 of the light chain variable region sequence set forth in SEQ ID NO:191, and a heavy chain variable region comprising a CDR1, a CDR2, and a CDR3 of the heavy chain variable region sequence set forth in SEQ ID NO:192;
(bx) a light chain variable region comprising a CDR1, a CDR2, and a CDR3 of the light chain variable region sequence set forth in SEQ ID NO:195, and a heavy chain variable region comprising a CDR1, a CDR2, and a CDR3 of the heavy chain variable region sequence set forth in SEQ ID NO:196;
(by) a light chain variable region comprising a CDR1, a CDR2, and a CDR3 of the light chain variable region sequence set forth in SEQ ID NO:199, and a heavy chain variable region comprising a CDR1, a CDR2, and a CDR3 of the heavy chain variable region sequence set forth in SEQ ID NO:200;
(bz) a light chain variable region comprising a CDR1, a CDR2, and a CDR3 of the light chain variable region sequence set forth in SEQ ID NO:203, and a heavy chain variable region comprising a CDR1, a CDR2, and a CDR3 of the heavy chain variable region sequence set forth in SEQ ID NO:204;
(ca) a light chain variable region comprising a CDR1, a CDR2, and a CDR3 of the light chain variable region sequence set forth in SEQ ID NO:207, and a heavy chain variable region comprising a CDR1, a CDR2, and a CDR3 of the heavy chain variable region sequence set forth in SEQ ID NO:208;
(cb) a light chain variable region comprising a CDR1, a CDR2, and a CDR3 of the light chain variable region sequence set forth in SEQ ID NO:211, and a heavy chain variable region comprising a CDR1, a CDR2, and a CDR3 of the heavy chain variable region sequence set forth in SEQ ID NO:212;
(cc) a light chain variable region comprising a CDR1, a CDR2, and a CDR3 of the light chain variable region sequence set forth in SEQ ID NO:215, and a heavy chain variable region comprising a CDR1, a CDR2, and a CDR3 of the heavy chain variable region sequence set forth in SEQ ID NO:216;
(cd) a light chain variable region comprising a CDR1, a CDR2, and a CDR3 of the light chain variable region sequence set forth in SEQ ID NO:219, and a heavy chain variable region comprising a CDR1, a CDR2, and a CDR3 of the heavy chain variable region sequence set forth in SEQ ID NO:220;
(ce) a light chain variable region comprising a CDR1, a CDR2, and a CDR3 of the light chain variable region sequence set forth in SEQ ID NO:223, and a heavy chain variable region comprising a CDR1, a CDR2, and a CDR3 of the heavy chain variable region sequence set forth in SEQ ID NO:224;
(cf) a light chain variable region comprising a CDR1, a CDR2, and a CDR3 of the light chain variable region sequence set forth in SEQ ID NO:227, and a heavy chain variable region comprising a CDR1, a CDR2, and a CDR3 of the heavy chain variable region sequence set forth in SEQ ID NO:228;
(cg) a light chain variable region comprising a CDR1, a CDR2, and a CDR3 of the light chain variable region sequence set forth in SEQ ID NO:231, and a heavy chain variable region comprising a CDR1, a CDR2, and a CDR3 of the heavy chain variable region sequence set forth in SEQ ID NO:232;
(ch) a light chain variable region comprising a CDR1, a CDR2, and a CDR3 of the light chain variable region sequence set forth in SEQ ID NO:235, and a heavy chain variable region comprising a CDR1, a CDR2, and a CDR3 of the heavy chain variable region sequence set forth in SEQ ID NO:236;
(ci) a light chain variable region comprising a CDR1, a CDR2, and a CDR3 of the light chain variable region sequence set forth in SEQ ID NO:239, and a heavy chain variable region comprising a CDR1, a CDR2, and a CDR3 of the heavy chain variable region sequence set forth in SEQ ID NO:240;
(cj) a light chain variable region comprising a CDR1, a CDR2, and a CDR3 of the light chain variable region sequence set forth in SEQ ID NO:243, and a heavy chain variable region comprising a CDR1, a CDR2, and a CDR3 of the heavy chain variable region sequence set forth in SEQ ID NO:244;
(ck) a light chain variable region comprising a CDR1, a CDR2, and a CDR3 of the light chain variable region sequence set forth in SEQ ID NO:247, and a heavy chain variable region comprising a CDR1, a CDR2, and a CDR3 of the heavy chain variable region sequence set forth in SEQ ID NO:248;
(cl) a light chain variable region comprising a CDR1, a CDR2, and a CDR3 of the light chain variable region sequence set forth in SEQ ID NO:251, and a heavy chain variable region comprising a CDR1, a CDR2, and a CDR3 of the heavy chain variable region sequence set forth in SEQ ID NO:252;
(cm) a light chain variable region comprising a CDR1, a CDR2, and a CDR3 of the light chain variable region sequence set forth in SEQ ID NO:255, and a heavy chain variable region comprising a CDR1, a CDR2, and a CDR3 of the heavy chain variable region sequence set forth in SEQ ID NO:256;
(cn) a light chain variable region comprising a CDR1, a CDR2, and a CDR3 of the light chain variable region sequence set forth in SEQ ID NO:259, and a heavy chain variable region comprising a CDR1, a CDR2, and a CDR3 of the heavy chain variable region sequence set forth in SEQ ID NO:260;
(co) a light chain variable region comprising a CDR1, a CDR2, and a CDR3 of the light chain variable region sequence set forth in SEQ ID NO:263, and a heavy chain variable region comprising a CDR1, a CDR2, and a CDR3 of the heavy chain variable region sequence set forth in SEQ ID NO:264;
(cp) a light chain variable region comprising a CDR1, a CDR2, and a CDR3 of the light chain variable region sequence set forth in SEQ ID NO:267, and a heavy chain variable region comprising a CDR1, a CDR2, and a CDR3 of the heavy chain variable region sequence set forth in SEQ ID NO:268;
(cq) a light chain variable region comprising a CDR1, a CDR2, and a CDR3 of the light chain variable region sequence set forth in SEQ ID NO:271, and a heavy chain variable region comprising a CDR1, a CDR2, and a CDR3 of the heavy chain variable region sequence set forth in SEQ ID NO:272;
(cr) a light chain variable region comprising a CDR1, a CDR2, and a CDR3 of the light chain variable region sequence set forth in SEQ ID NO:275, and a heavy chain variable region comprising a CDR1, a CDR2, and a CDR3 of the heavy chain variable region sequence set forth in SEQ ID NO:276;
(cs) a light chain variable region comprising a CDR1, a CDR2, and a CDR3 of the light chain variable region sequence set forth in SEQ ID NO:279, and a heavy chain variable region comprising a CDR1, a CDR2, and a CDR3 of the heavy chain variable region sequence set forth in SEQ ID NO:280;
(ct) a light chain variable region comprising a CDR1, a CDR2, and a CDR3 of the light chain variable region sequence set forth in SEQ ID NO:283, and a heavy chain variable region comprising a CDR1, a CDR2, and a CDR3 of the heavy chain variable region sequence set forth in SEQ ID NO:284;
(cu) a light chain variable region comprising a CDR1, a CDR2, and a CDR3 of the light chain variable region sequence set forth in SEQ ID NO:287, and a heavy chain variable region comprising a CDR1, a CDR2, and a CDR3 of the heavy chain variable region sequence set forth in SEQ ID NO:288;
(cv) a light chain variable region comprising a CDR1, a CDR2, and a CDR3 of the light chain variable region sequence set forth in SEQ ID NO:291, and a heavy chain variable region comprising a CDR1, a CDR2, and a CDR3 of the heavy chain variable region sequence set forth in SEQ ID NO:292;
(cw) a light chain variable region comprising a CDR1, a CDR2, and a CDR3 of the light chain variable region sequence set forth in SEQ ID NO:303, and a heavy chain variable region comprising a CDR1, a CDR2, and a CDR3 of the heavy chain variable region sequence set forth in SEQ ID NO:304;
(cx) a light chain variable region comprising a CDR1, a CDR2, and a CDR3 of the light chain variable region sequence set forth in SEQ ID NO:295, and a heavy chain variable region comprising a CDR1, a CDR2, and a CDR3 of the heavy chain variable region sequence set forth in SEQ ID NO:296;
(cy) a light chain variable region comprising a CDR1, a CDR2, and a CDR3 of the light chain variable region sequence set forth in SEQ ID NO:917, and a heavy chain variable region comprising a CDR1, a CDR2, and a CDR3 of the heavy chain variable region sequence set forth in SEQ ID NO:915; or
(cz) a light chain variable region comprising a CDR1, a CDR2, and a CDR3 of the light chain variable region sequence set forth in SEQ ID NO:921, and a heavy chain variable region comprising a CDR1, a CDR2, and a CDR3 of the heavy chain variable region sequence set forth in SEQ ID NO:919, thereby inducing tumor cell death in the subject.

21. The method of claim 20, wherein the tumor is selected from the group consisting of multiple myeloma, Non-Hodgkin Lymphoma, Mantle Cell Lymphoma, Hodgkin Lymphoma, Chronic Lymphocytic Leukemia (CLL), Acute lymphocytic leukemia (ALL), Hairy Cell Leukemia, Burkitt's Lymphoma, and Waldenstrom's Macroglobulinemia.

22. The method of claim 20, wherein the immunoresponsive cell is a T cell.
